(19) United States
(12) Patent Application Publication
(10) Pub. No.: US 2025/0304576 A1
(43) Pub. Date: Oct. 2, 2025

(54) IKK INHIBITORS

(71) Applicant: Cancer Research Technology Limited, London (GB)

(72) Inventors: Simon P. MacKay, Glasgow (GB); Aisha A. Alsfouk, Riyadh (SA); Usama Ammar, Edinburgh (GB); Nahoum G. Anthony, Newcastle upon Tyne (GB); Jessica Baiget; Giacomo Berretta; David Breen; Judith Huggan, Dalry (GB); Christopher Lawson; Domiziana Masci, Rome (IT); Sabin Llona-Minguez; Christopher Riley, Glasgow (GB); Colin J. Suckling; Christopher West, Glasgow (GB)

(21) Appl. No.: 18/863,263

(22) PCT Filed: May 11, 2023
(86) PCT No.: PCT/GB2023/051242

§ 371 Date: Nov. 5, 2024

(30) Foreign Application Priority Data
May 11, 2022 (GB) 2206901.7

(51) Int. Cl. C07D 471/04; A61K 31/437; A61K 31/4439; A61K 31/444; A61K 31/4545; A61K 31/496; A61K 31/506; A61K 31/5377; A61K 31/551; A61P 35/00; C07D 401/04; C07D 401/14; C07D 403/04; C07D 405/14; C07D 409/14; C07D 413/14; C07D 417/14

(52) U.S. Cl. CPC C07D 471/04; A61K 31/437; A61K 31/4439; A61K 31/444; A61K 31/4545; A61K 31/496; A61K 31/506; A61K 31/5377; A61K 31/551; A61P 35/00; C07D 401/04; C07D 401/14; C07D 403/04; C07D 405/14; C07D 409/14; C07D 413/14; C07D 417/14

(57) ABSTRACT

The present invention relates to IKK-alpha inhibitory compounds, or a pharmaceutically acceptable salts, hydrates or solvates thereof, having the structural Formula (I), shown below:

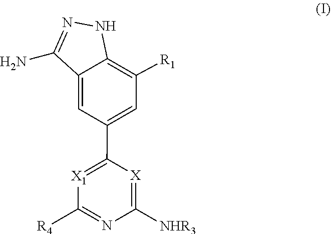

wherein R₁, X, R₃ R₄ and X₁ are each as defined herein. The present invention also relates to processes for the preparation of these compounds, to pharmaceutical compositions comprising them, and to their use in the treatment of proliferative disorders, such as cancer, as well as other diseases or conditions in which IKK-alpha activity is implicated.

### INTRODUCTION

The present invention relates to certain compounds that function as inhibitors of inhibitory-KB kinase (IKK) activity, and especially the alpha subunit of IKK (IKKα). The compounds of the present invention may therefore be used to treat disease or conditions mediated, at least in part, by aberrant or inappropriate IKK (and especially IKKα) activity. Cancer is an example of condition associated with aberrant or inappropriate IKK (and especially IKKα) activity. The invention furthermore relates to the use of the compounds as for treating diseases or conditions in which IKK (and especially IKKα) activity is implicated, to processes for making these compounds and to pharmaceutical compositions comprising them.

### BACKGROUND OF THE INVENTION

Cancer is caused by altered cellular proliferation. Precisely what causes a cell to become malignant and proliferate in an uncontrolled and unregulated manner has been the focus of intense research over recent decades. This research has led to the identification of molecular targets associated with key pathways that enable such malignancies.

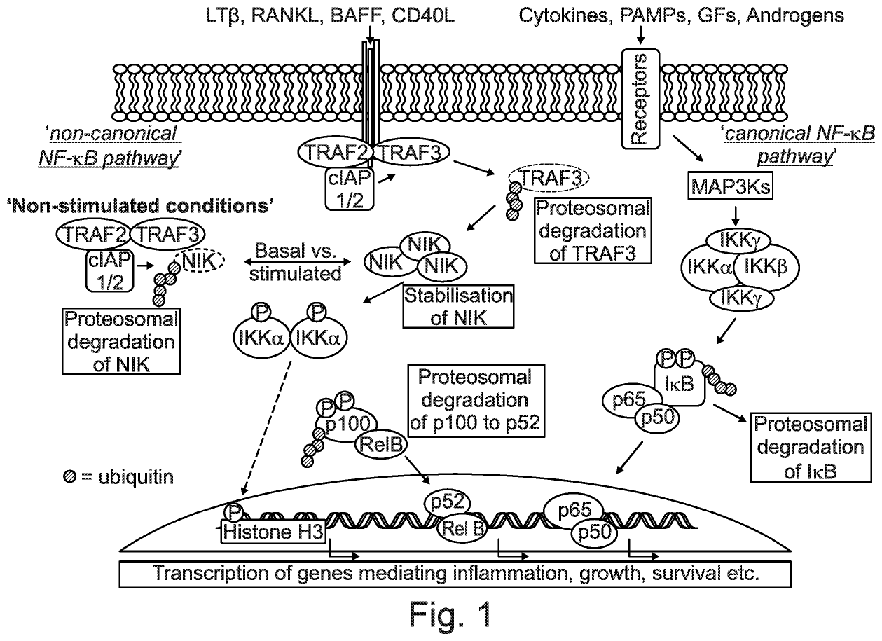

Nuclear Factor kappa-B (NF-κB), from Nuclear factor kappa-light-chain enhancer of B-cells, represents a family of five transcription factors involved in diverse biological responses that underpin phenotypic outcomes of inflammation, modulation of immune responses, cell growth, proliferation, apoptosis and aspects of differentiation and development [1-5]. NF-κB signalling is now appreciated as either canonical (classical) or non-canonical (alternative) pathways via the mobilisation of both homo- and hetero-dimer complexes of these family members (FIG. 1; [1-5]). Collectively the NF-κB proteins are five distinct isoforms; RelA (p65), RelB, c-Rel, NF-κB1 (p105/p50) and NF-κB2 (p100/p52) [1-5]. In an inactive state these proteins are typically associated with inhibitory-κB (IκB) proteins, including isoforms of IκBa, IκB3, and IκBε and in the case of p105 and p100 proteins it is their intrinsic protein structure that maintains them in a self-bound inhibitory form by virtue of their C-terminal IκB-like structures (IκBδ and IκBγ respectively) composed of ankyrin repeats [1-5]. Activation and liberation of NF-κB proteins occurs typically in response to a number of extracellular ligands, as well as agents that generate a DNA Damage response (DDR), resulting in the nuclear localisation of DNA-binding protein dimers following dissociation from IκB molecules [1-5].

The canonical pathway can be activated in response to cytokines such as TNFα and IL-1β, and pathogen-associated molecular profiles (PAMPs) such as the bacterial endotoxin lipopolysaccharide (LPS) [6, 7]. This response is typically rapid and transient, mediated by the classical inhibitory-κB kinase (IKK) complex (IKKα/β/γ) with a requirement for IKKβ-mediated phosphorylation of selected IκB proteins [6, 7]. In contrast, activation of the non-canonical NF-κB pathway is relatively slower and over a period of hours results in an IKKα-mediated liberation of predominately p52-RelB dimers to drive gene transcription [1-7]. This slower response reflects reliance upon protein expression/stabilisation within the upstream components of the pathway. Whilst TNFα and IL-13 have the ability to activate the non-canonical NF-κB pathway it is typically alternative members of the greater TNF superfamily that drive activation [3, 4]. This includes lymphotoxin-3 (LT-p), the related tumor necrosis factor superfamily member 14 (TNFSF14) known as LIGHT, TNF-like weak inducer of apoptosis (TWEAK), CD40 ligand (CD40L), Receptor-activator of NF-κB ligand (RANKL) and B-cell activating factor (BAFF) [1, 3, 4].

A combination of molecular and genetic studies has shown that receptor mediated-non-canonical NF-κB activation is built around the paradigm of a TNF super family ligand activating its cognate receptor via recruitment of a sequence of identifiable adaptor molecules of the TNF-Receptor associated factor (TRAF) family, notably TRAF2 and TRAF3, modulators of ubiquitination and associated protein degradation in the form of the cellular inhibitors of apoptosis (clAPs). These proteins enable engagement and activation of the cellular kinases NF-κB-inducing kinase (NIK), the 14^{th} member of the MAP kinase kinase kinase (MAP3K) family, and IKKα to determine the liberation of p52-RelB protein complexes (FIG. 1; [1-5]).

In a cellular setting, under resting non-stimulated conditions, NIK is maintained at a low expression level based upon NIK-focused proteasomal degradation. However, upon receptor activation NIK is stabilised, protein expression is increased to enable pathway activation [16]. It is TRAF3 that acts as the crucial regulator of NIK expression by controlling the extent of its proteasome-mediated degradation [16]. Upon receptor activation the focus of proteasome-mediated protein degradation switches from NIK to that of TRAF2 and TRAF3 which stabilises NIK expression to initiate the sequence of signalling events toward p100 processing [17-20]. The clAP proteins that function as ubiquitin ligases to ubiquitinate NIK then target TRAF3 for degradation to increase NIK protein levels.

Upon NIK protein stabilisation, as the first component of the non-canonical NF-κB pathway it catalyses is the phosphorylation of IKKα and supports IKKα recruitment to and phosphorylation of p100 to drive subsequent p100 ubiquitination and proteasome-mediated degradation to liberate p52 [16]. Under basal conditions p100 exists typically in dimer complexes with RelB and upon stimulated degradation generates p52-RelB dimers able to translocate to the nucleus to initiate the transcription of distinct genes (FIG. 1).

Both NIK and IKKα play critical roles in the phosphorylation of p100 to liberate mature p52-RelB protein dimers. However, whilst IKKα is now viewed as the key modulator of p100 phosphorylation there is a co-dependence on NIK to deliver coupled phosphorylation and processing of p100 to generate mature p52 that is transcriptionally active [22]. In transfected cells, NIK can stimulate the phosphorylation, ubiquitination and processing of p100 [23, 24], however recombinant NIK itself does not display any phosphorylation of p100 in vitro [24, 25]. In the cell-based setting, NIK mediates downstream signalling by engaging and activating IKKα resulting in the phosphorylation of the C-terminal region of p100 [25], and this is independent of the other IKK isoforms, β and γ associated with canonical NF-κB activation [26, 27]. Whilst IKKα phosphorylates p100 and regulated non-canonical NF-κB activation alone, it is not as effective at inducing p100 processing as NIK [23]. With these observations, further studies then identified NIK to have a critical role in regulating p100 processing via the recruitment of IKKα to and binding with p100 as a protein substrate [22]. Collectively, NIK-IKKα interaction with p100 results in the phosphorylation of p100 at specific serine residues, primarily Ser868/870 [24]. These sites are components of the phospho-degron within the p100 C-terminal NIK-responsive domain (NRD) and when phosphorylated lead to βTrCP binding as part of the SCF^{βTrCP} ubiquitin ligase complex that drives the eventual processing of p100 to generate p52.

Independent of the non-canonical NF-κB pathway, a number of studies have identified that at the NIK-IKKα kinase level there are also examples of signal bifurcation. These can be dependent on differing extracellular conditions [29] and demonstrate that p100 is not the only substrate for IKKα-mediated phosphorylation. IKKα via catalysed phosphorylation, regulates directly a number of cellular proteins that then either directly or indirectly regulate cellular transcription [6, 7]. This includes transcription factors distinct from the NF-κB family, for example E2F1 [30, 31], β-catenin [32], CBP [33], as well as the suppressors of transcription such as the silencing mediator for retinoic acid and thyroid hormone receptor (SMRT) [34] and cell cycle regulator cyclin D1 [35]. Additional substrates also include the Protein inhibitor of activated STAT1 (PIAS1) as a modulator of transcription/inflammation [36], the oestrogen (ER) [37] and androgen receptors (AR) [38] of the steroid hormone family receptor along with their associated steroid receptor co-factor (SRC)-3 [37, 39, 40] and Aurora kinase A [41, 42] that contributes to the mitotic process. Direct modulation of the status of these proteins by IKKα has bearing on the transcription of additional regulatory proteins such as p53 [43, 44] and EZH2 [44] and additional mitotic kinase Polo-like kinase (PLK) 4 [45]. IKKα therefore serves as a key switch in the coordinated regulation of both NF-κB-dependent and NF-κB-independent gene transcription and this underpins the outcomes associated with events that initiate and/or perpetuate the development of acquired characteristics, or phenotypes, we now recognise as cancer ‘Hallmarks’ as identified and defined by Hanahan & Weinberg [46, 47]. The transcriptional modulation driven by IKKα-mediated signalling, divulged using a number of experimental approaches such as genetic deletion and reconstitution [48, 49], siRNA ‘run-down’ [35] and over-expression strategies [50], may be in excess of 200 genes and these gene/protein induction/repression events support the acquisition of characteristics of specific ‘Hallmarks’, particularly the ability of tumours to ‘sustain proliferative signalling’, ‘resist cell death’, ‘evade growth suppressors’ and encourage ‘genomic instability and mutation’. More striking is the role of IKKα in regulating genes/protein that help to underpin the phenotypes associated with longer term tumour development: ‘inducing angiogenesis’ and ‘activating invasion and metastasis’ byway of regulating cytokine (e.g. IL-13, IL-6 [48, 49]) and chemokine (e.g. CCL19, CCL21, CXCL12, CXCL13 and BAFF [27, 51, 52]) induction and modulation of adhesion molecule (e.g. VCAM; [48-50]), maspin [50; 53] and MMPs [50] expression in different cellular/tissue situations. It is also evident that in particular sub-types of cancer the acquisition of a specific mutation, C250T in the hTERT promoter [54] that supports tumour reactivation has identified the potential for tumours to become ‘addicted’ to IKKα-mediated non-canonical NF-κB signalling thus ‘enabling replicative potential’. Collectively, perturbation of this enzyme could have wide-ranging effects on the multiple hallmarks of tumour cells described above. Moreover, given the impact of IKKα in regulating major cytokine, chemokine and matrix metalloproteinase isoforms, intervention against this enzyme may have significant effects on tumour-stromal communication and matrix composition within the tumour microenvironment and define a better understanding of ‘tumour-promoting inflammation’.

Additional complexities to the regulation of IKKα-dependent, NF-κB-dependent and -independent gene transcription are also now apparent in the cancer setting, as we now appreciate that this transcriptional process is not wholly driven by receptor-mediated activation. For both solid tumour (e.g. pancreatic adenocarcinomas) and haematological settings (e.g. multiple myeloma) constitutive activation of IKKα-mediated signalling has been reported as a result of modulation of expression of upstream TRAF and clAP components in the pathways or mutation in these very same components that ultimately results in constitutive activation of the pathway in the absence of agonist. Furthermore, a truncated p45 form of IKKα has been identified in a number of colorectal cancers [55, 56], particularly those with a recognised B-Raf^{V600E} mutant background. This drives p45 IKKα-mediated nuclear signalling in a TNF superfamily member-independent manner and so brings additional mechanistic and transcriptional diversity to tumour development, which has implications for potential intervention therapeutically.

In recent years the role of the non-canonical NF-κB pathway and IKKα within it have increasingly been implicated in the development and progression of multiple solid tumours and haematological cancers. As a consequence, there is a need and a desire to identify potentially useful IKKα inhibitors

The present invention was devised with the foregoing in mind.

### REFERENCES

[1] Sun S C. Non-canonical NF-κB signaling pathway. Cell Res. 2011 January; 21(1):71-85.
[2] Razani B, Reichardt A D, Cheng G. Non-canonical NF-κB signaling activation and regulation: principles and perspectives. Immunol Rev. 2011 November; 244(1):44-54.
[3] Cildir G, Low K C, Tergaonkar V. Noncanonical NF-κB Signaling in Health and Disease. Trends Mol Med. 2016 May; 22(5):414-429.
[4] Sun S C. The non-canonical NF-κB pathway in immunity and inflammation. Nat Rev Immunol. 2017 September; 17(9):545-558.
[5] Xia L, Tan S, Zhou Y, Lin J, Wang H, Oyang L, Tian Y, Liu L, Su M, Wang H, Cao D, Liao Q. Role of the NFκB-signaling pathway in cancer. Onco Targets Ther. 2018 Apr. 11; 11:2063-2073.
[6] Perkins N D. Integrating cell-signalling pathways with NF-kappaB and IKK function. Nat Rev Mol Cell Biol. 2007 January; 8(1):49-62.
[7] Gamble C, McIntosh K, Scott R, Ho K H, Plevin R, Paul A. Inhibitory kappa B Kinases as targets for pharmacological regulation. Br J Pharmacol. 2012 February; 165(4):802-19.
[16] Liao G, Zhang M, Harhaj E W, Sun S C. Regulation of the NF-kappaB-inducing kinase by tumor necrosis factor receptor-associated factor 3-induced degradation. J Biol Chem. 2004 Jun. 18; 279(25):26243-50.
[17] Vallabhapurapu S, Matsuzawa A, Zhang W, Tseng P H, Keats J J, Wang H, Vignali D A, Bergsagel P L, Karin M. Nonredundant and complementary functions of TRAF2 and TRAF3 in a ubiquitination cascade that activates NIK-dependent alternative NF-kappaB signaling. Nat Immunol. 2008 December; 9(12):1364-70.
[18] Vince J E, Wong W W, Khan N, Feltham R, Chau D, Ahmed A U, Benetatos C A, Chunduru S K, Condon S M, McKinlay M, Brink R, Leverkus M, Tergaonkar V, Schneider P, Callus B A, Koentgen F, Vaux D L, Silke J. IAP antagonists target cIAP1 to induce TNFalpha-dependent apoptosis. Cell. 2007 Nov. 16; 131(4):682-93.
[19] Varfolomeev E, Blankenship J W, Wayson S M, Fedorova A V, Kayagaki N, Garg P, Zobel K, Dynek J N, Elliott L O, Wallweber H J, Flygare J A, Fairbrother W J, Deshayes K, Dixit V M, Vucic D. IAP antagonists induce autoubiquitination of c-IAPs, NF-kappaB activation, and TNFalpha-dependent apoptosis. Cell. 2007 Nov. 16; 131(4):669-81
[20] Zarnegar B J, Wang Y, Mahoney D J, Dempsey P W, Cheung H H, He J, Shiba T, Yang X, Yeh W C, Mak T W, Korneluk R G, Cheng G. Noncanonical NF-kappaB activation requires coordinated assembly of a regulatory complex of the adaptors cIAP1, cIAP2, TRAF2 and TRAF3 and the kinase NIK. Nat Immunol. 2008 December; 9(12):1371-8
[22] Xiao G, Fong A, Sun S C. Induction of p100 processing by NF-kappaB-inducing kinase involves docking IkappaB kinase alpha (IKKalpha) to p100 and IKKalpha-mediated phosphorylation. J Biol Chem. 2004 Jul. 16; 279(29):30099-105
[23] Xiao G, Harhaj E W, Sun S C. NF-kappaB-inducing kinase regulates the processing of NF-kappaB2 p100. Mol Cell. 2001 February; 7(2):401-9. [24] Liang C, Zhang M, Sun S C. beta-TrCP binding and processing of NF-kappaB2/p100 involve its phosphorylation at serines 866 and 870. Cell Signal. 2006 August; 18(8):1309-17
[25] Senftleben U, Cao Y, Xiao G, Greten F R, Krshn G, Bonizzi G, Chen Y, Hu Y, Fong A, Sun S C, Karin M. Activation by IKKalpha of a second, evolutionary conserved, NF-kappa B signaling pathway. Science. 2001 Aug. 24; 293(5534):1495-9.
[26] Claudio E, Brown K, Park S, Wang H, Siebenlist U. BAFF-induced NEMO-independent processing of NF-kappa B2 in maturing B cells. Nat Immunol. 2002 October; 3(10):958-65
[27] Dejardin E, Droin N M, Delhase M, Haas E, Cao Y, Makris C, Li Z W, Karin M, Ware C F, Green D R. The lymphotoxin-beta receptor induces different patterns of gene expression via two NF-kappaB pathways. Immunity. 2002 October; 17(4):525-35.
[29] Wang R P, Zhang M, Li Y, Diao F C, Chen D, Zhai Z, Shu H B. Differential regulation of IKK alpha-mediated activation of IRF3/7 by NIK. Mol Immunol. 2008 April; 45(7):1926-34.
[30] Tu Z, Prajapati S, Park K J, Kelly N J, Yamamoto Y, Gaynor R B. IKK alpha regulates estrogen-induced cell cycle progression by modulating E2F1 expression. J Biol Chem. 2006 Mar. 10; 281(10):6699-706.
[31] Ammirante M, Kuraishy Al, Shalapour S, Strasner A, Ramirez-Sanchez C, Zhang W, Shabaik A, Karin M. An IKKα-E2F1-BMl1 cascade activated by infiltrating B cells controls prostate regeneration and tumor recurrence. Genes Dev. 2013 Jul. 1; 27(13):1435-40.
[32] Lamberti C, Lin K M, Yamamoto Y, Verma U, Verma I M, Byers S, Gaynor R B. Regulation of beta-catenin function by the IkappaB kinases. J Biol Chem. 2001 Nov. 9; 276(45):42276-86
[33] Huang W C, Ju T K, Hung M C, Chen C C. Phosphorylation of CBP by IKKalpha promotes cell growth by switching the binding preference of CBP from p53 to NF-kappaB. Mol Cell. 2007 Apr. 13; 26(1):75-87
[34] Hoberg J E, Popko A E, Ramsey C S, Mayo M W. IkappaB kinase alpha-mediated derepression of SMRT potentiates acetylation of RelA/p65 by p300. Mol Cell Biol. 2006 January; 26(2):457-71.
[35] Kwak Y T, Li R, Becerra C R, Tripathy D, Frenkel E P, Verma U N. IkappaB kinase alpha regulates subcellular distribution and turnover of cyclin D1 by phosphorylation. J Biol Chem. 2005 Oct. 7; 280(40):33945-52.
[36] Liu B, Yang Y, Chernishof V, Loo R R, Jang H, Tahk S, Yang R, Mink S, Shultz D, Bellone C J, Loo J A, Shuai K. Proinflammatory stimuli induce IKKalpha-mediated phosphorylation of PIAS1 to restrict inflammation and immunity. Cell. 2007 Jun. 1; 129(5):903-14
[37] Park K J, Krishnan V, O'Malley B W, Yamamoto Y, Gaynor R B. Formation of an IKKalpha-dependent transcription complex is required for estrogen receptor-mediated gene activation. Mol Cell. 2005 Apr. 1; 18(1):71-82
[38] Jain G, Voogdt C, Tobias A, Spindler K D, Müller P, Cronauer M V, Marienfeld R B. IκB kinases modulate the activity of the androgen receptor in prostate carcinoma cell lines. Neoplasia. 2012 March; 14(3):178-89.
[39] Wu R C, Qin J, Hashimoto Y, Wong J, Xu J, Tsai S Y, Tsai M J, O'Malley B W. Regulation of SRC-3 (pCIP/ACTR/AIB-1/RAC-3/TRAM-1) Coactivator activity by I kappa B kinase. Mol Cell Biol. 2002 May; 22(10):3549-61.
[40] Wu R C, Qin J, Yi P, Wong J, Tsai S Y, Tsai M J, O'Malley B W. Selective phosphorylations of the SRC-3/AIB1 coactivator integrate genomic reponses to multiple cellular signaling pathways. Mol Cell. 2004 Sep. 24; 15(6):937-49.
[41] Prajapati S, Tu Z, Yamamoto Y, Gaynor R B. IKKalpha regulates the mitotic phase of the cell cycle by modulating Aurora A phosphorylation. Cell Cycle. 2006 October; 5(20):2371-80.
[42] Irelan J T, Murphy T J, DeJesus P D, Teo H, Xu D, Gomez-Ferreria M A, Zhou Y, Miraglia L J, Rines D R, Verma I M, Sharp D J, Tergaonkar V, Chanda S K. A role for IkappaB kinase 2 in bipolar spindle assembly. Proc Natl Acad Sci USA. 2007 Oct. 23; 104(43):16940-5.
[43] Schumm K, Rocha S, Caamano J, Perkins N D. Regulation of p53 tumour suppressor target gene expression by the p52 NF-kappaB subunit. EMBO J. 2006 Oct. 18; 25(20):4820-32.
[44] lannetti A, Ledoux A C, Tudhope S J, Sellier H, Zhao B, Mowla S, Moore A, Hummerich H, Gewurz B E, Cockell S J, Jat P S, Willmore E, Perkins N D. Regulation of p53 and Rb links the alternative NF-κB pathway to EZH2 expression and cell senescence. PLoS Genet. 2014 Sep. 25; 10 (9):e1004642
[45] Ledoux A C, Sellier H, Gillies K, lannetti A, James J, Perkins N D. NFκB regulates expression of Polo-like kinase 4. Cell Cycle. 2013 Sep. 15; 12(18):3052-62
[46] Hanahan D, Weinberg R A. The hallmarks of cancer. Cell. 2000 Jan. 7; 100(1):57-70.
[47] Hanahan D, Weinberg R A. Hallmarks of cancer: the next generation. Cell. 2011 Mar. 4; 144(5):646-74.
[48] Li X, Massa P E, Hanidu A, Peet G W, Aro P, Savitt A, Mische S, Li J, Marcu K B. IKKalpha, IKKbeta, and NEMO/IKKgamma are each required for the NF-kappa B-mediated inflammatory response program. J Biol Chem. 2002 Nov. 22; 277(47):45129-40.
[49] Massa P E, Li X, Hanidu A, Siamas J, Pariali M, Pareja J, Savitt A G, Catron K M, Li J, Marcu K B. Gene expression profiling in conjunction with physiological rescues of IKKalpha-null cells with wild type or mutant IKKalpha reveals distinct classes of IKKalpha/NF-kappaB-dependent genes. J Biol Chem. 2005 Apr. 8; 280(14):14057-69
[50] Nadiminty N, Dutt S, Tepper C, Gao A C. Microarray analysis reveals potential target genes of NF-kappaB2/p52 in LNCaP prostate cancer cells. Prostate. 2010 Feb. 15; 70(3):276-87.
[51] Wharry C E, Haines K M, Carroll R G, May M J. Constitutive non-canonical NFkappaB signaling in pancreatic cancer cells. Cancer Biol Ther. 2009 August; 8(16):1567-76.
[52] Ammirante M, Shalapour S, Kang Y, Jamieson C A, Karin M. Tissue injury and hypoxia promote malignant progression of prostate cancer by inducing CXCL13 expression in tumor myofibroblasts. Proc Natl Acad Sci USA. 2014 Oct. 14; 111(41):14776-81.
[53] Ammirante M, Luo J L, Grivennikov S, Nedospasov S, Karin M. B-cell-derived lymphotoxin promotes castration-resistant prostate cancer. Nature. 2010 Mar. 11; 464(7286):302-5.
[54] Li Y, Zhou Q L, Sun W, Chandrasekharan P, Cheng H S, Ying Z, Lakshmanan M, Raju A, Tenen D G, Cheng S Y, Chuang K H, Li J, Prabhakar S, Li M, Tergaonkar V. Non-canonical NF-κB signalling and ETS1/2 cooperatively drive C250T mutant TERT promoter activation. Nat Cell Biol. 2015 October; 17(10):1327-38.
[55] Margalef P, Fernendez-Majada V, Villanueva A, Garcia-Carbonell R, Iglesias M, López L, Martinez-Iniesta M, Villa-Freixa J, Mulero M C, Andreu M, Torres F, Mayo M W, Bigas A, Espinosa L. A truncated form of IKKα is responsible for specific nuclear IKK activity in colorectal cancer. Cell Rep. 2012 Oct. 25; 2(4):840-54.
[56] Margalef P, Colomer C, Villanueva A, Montagut C, Iglesias M, Bellosillo B, Salazar R, Martinez-Iniesta M, Bigas A, Espinosa L. BRAF-induced tumorigenesis is IKKα-dependent but NF-κB-independent. Sci Signal. 2015 Apr. 21; 8 (373):ra38.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting IKKα activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which IKKα activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, as defined herein for use in the inhibition of IKKα activity.

According to a further aspect of the present invention, there is provided a compound, or a pharmaceutically acceptable salt, hydrate or solvate thereof, as defined herein for use in the treatment of a disease or disorder in which IKKα activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided a use of a compound, or a pharmaceutically acceptable salt, hydrate or solvate thereof, as defined herein in the manufacture of a medicament for the inhibition of IKKα activity.

According to a further aspect of the present invention, there is provided a use of a compound, or a pharmaceutically acceptable salt, hydrate or solvate thereof, as defined herein in the manufacture of a medicament for the treatment of a disease or disorder in which IKKα activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to compounds, or pharmaceutically acceptable salts, hydrates or solvates thereof, having the structural Formula (I), shown below:

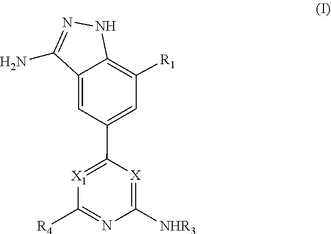

wherein:
R₁ is selected from hydrogen, halogen, (1-6C)alkyl, (2-6C)alkynyl, (3-7C) cycloalkyl, aryl, heteroaryl and heterocyclyl,
   and wherein said (1-6C)alkyl, (2-6C)alkynyl, (3-7C) cycloalkyl, aryl, heteroaryl and heterocyclyl are optionally substituted by one or more R₁₀₀ substituents;
   wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}heterocyclyl, (CH₂)_{z}heteroaryl, or (CH₂)_{z}aryl;
   and wherein:
      (i) R_{f} and R_{g} are each independently selected from hydrogen, (1-6C)alkyl or phenyl; and wherein Rₕ and Rⱼ are each independently selected from hydrogen, (1-6C)alkyl or phenyl or Rₕ and Rⱼ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (1-4C)alkyl, (3-7C)cycloalkyl, heterocyclyl, heteroaryl or aryl in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl;
X is N or CR₂; wherein R₂ is selected from hydrogen, halogen, (1-8C)alkyl, (2-8C)alkenyl, (2-8C)alkynyl, (3-7C)cycloalkyl, aryl, heteroaryl and heterocyclyl,
   wherein said (1-8C)alkyl, (2-8C)alkenyl, (2-8C)alkynyl, (3-7C)cycloalkyl, aryl, heteroaryl and heterocyclyl are optionally substituted by one or more R₂₀₀ substituents;
   wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)aryl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or aryl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl;
R₃ is selected from hydrogen, (1-8C)alkyl, (3-7C)cycloalkyl, (CH₂)₁₋₃ (3-7C)cycloalkyl, a carbon-linked 4 to 7 membered heterocyclyl, a carbon-linked 5 to 6 membered heteroaryl, —C(O)-(1-8C)alkyl, —C(O)(3-7C)cycloalkyl, —C(O)[5 or 6-membered heteroaryl], —C(O)phenyl, —C(O)O(1-8C)alkyl, C(O)O(3-7C)cycloalkyl, C(O)O(CH₂)₁₋₃(3-7C)cycloalkyl, —C(O)NH₂, —C(O)NH-(1-8C)alkyl, —C(O)NH—(CH₂)₀₋₃(3-7C)cycloalkyl, —C(O)NH—(CH₂)₀₋₃heterocyclyl, —C(O)NH—(CH₂)₀₋₃[5 or 6-membered heteroaryl], —C(O)NH—(CH₂)₀₋₃phenyl, —S(O)₂H or —S(O)₂-(1-8C)alkyl;
   wherein any alkyl, cycloalkyl, phenyl, heteroaryl or heterocyclyl moiety is optionally substituted by one or more R₃₀₀ substituents;
   wherein R₃₀₀ is selected from halo, trifluoromethyl, trifluoromethoxy, cyano, hydroxyl, (1-4C)alkyl, (1-4C)hydroxyalkyl, (CH₂)_{z}ORₛ, (CH₂)_{z}C(O)Rₛ, (CH₂)_{z}C(O)ORₛ, (CH₂)_{z}OC(O)Rₛ, (CH₂)_{z}C(O)N(Rᵥ)Rᵤ, (CH₂)_{z}N(Rₜ)C(O)Rₛ, (CH₂)_{z}N(Rₜ)C(O)ORₛ, (CH₂)_{z}S(O)_{y}Rₛ, (CH₂)_{z}SO₂N(Rᵥ)Rᵤ, (CH₂)_{z}N(Rₜ)SO₂Rₛ, (CH₂)_{z}NRᵤRᵥ;
   and wherein:
      (i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms, and wherein any 3-7 membered ring formed Rₒ and Rₚ, and any alkyl or phenyl group present for Rₛ, Rₜ, Rᵤ and Rᵥ is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any alkyl, cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{w}, C(O)R_{w}, C(O)OR_{w}, OC(O)R_{w}, C(O)N(R_{w})Rₓ, N(Rₓ)C(O)R_{w}, S(O)_{y}R_{w}, SO₂N(Rₓ)R_{w}, N(Rₓ)SO₂R_{w}, or NR_{w}Rₓ, wherein R_{w} is hydrogen, (1-2C)alkyl or phenyl, and Rₓ are selected from hydrogen or (1-2C)alkyl;
or R₂ and R₃ are linked such that together they form a —CH═CQ- or —N═CQ- group;
Q is hydrogen, halo, cyano or a group of the formula:

-L₁-Y₁-L₂-Q₁
wherein:
   L₁ is absent or (1-3C)alkylene;
   Y₁ is absent or O, S, SO, SO₂, N(R_{y1}), C(O), C(O)O, OC(O), C(O)N(R_{y1}), or
   N(R_{y1})C(O), wherein R_{y1} is selected from hydrogen or (1-4C)alkyl;
   L₂ is absent or (1-3C)alkylene; and
   Q₁ is hydrogen, (1-6C)alkyl, (2-6C)alkenyl, (2-6C)alkynyl, phenyl, (3-8C)cycloalkyl, heteroaryl or heterocyclyl;
   wherein Q is optionally further substituted by one or more substituent groups independently selected from oxo, hydroxy, (1-6C)alkyl, halo, (1-4C)haloalkyl, (1-4C)haloalkoxy, (1-4C)aminoalkyl, (1-4C)hydroxyalkyl, cyano, or by one or more group(s) of the formula:

-L₃-Y₂-L₄-W₁
wherein:
   L₃ is absent or (1-3C)alkylene;
   Y₂ is absent or selected from or O, S, SO, SO₂, N(R_{y2}), C(O), C(O)O, OC(O), C(O)N(R_{y2}), or N(R_{y2})C(O), S(O)₂N(R_{y2}), N(R_{y2})SO₂ wherein R_{y2} is selected from hydrogen or (1-3C)alkyl;
   L₄ is absent or (1-3C)alkylene; and
   W₁ is hydrogen, (1-6C)alkyl, phenyl, (3-8C)cycloalkyl, heteroaryl or heterocyclyl;
   wherein W₁ is optionally substituted by one or more substituents selected from oxo, (1-4C)alkyl, halo, (1-4C)haloalkyl, (1-4C)haloalkoxy, (1-4C)alkoxy, amino, (1-4C)alkylamino, di[(1-4C)alkyl]amino C(O)OH, C(O)O(1-4C)alkyl, (CH₂)₀₋₃-heterocyclyl or cyano;
R₄ is selected from hydrogen, halo, cyano or amino;
X₁ is N when R₂ and R₃ are linked such that together they form a —CH═CH— group; or CR₅ wherein R₅ is selected from hydrogen, halo, cyano or amino;
y is independently selected from 0, 1 or 2;
z is independently selected from 0, 1, 2 or 3;
   with the proviso that:
      (i) R₁ is not hydrogen when R₂ and R₃ are both hydrogen;
      (ii) R₂ is not hydrogen when R₁ and R₃ are both hydrogen;
      (iii) R₁ is not hydrogen when R₂ and R₃ are linked to form a —CH═CH— group;
      (iv) R₁ is not hydrogen when X is N and R₃ is hydrogen; and
      (v) Q₁ is not aryl or heteroaryl when all of L₁, Y₁ and L₂ are absent.

In a further aspect, the present invention relates to compounds of formula I defined above, wherein R₃ is selected from hydrogen, cyano, (1-8C)alkyl, (3-7C)cycloalkyl, (CH₂)₁₋₃(3-7C)cycloalkyl, a carbon-linked 4 to 7 membered heterocyclyl, a carbon-linked 5 to 6 membered heteroaryl, —C(O)-(1-8C)alkyl, —C(O)(CH₂)₀₋₃(3-7C)cycloalkyl, —C(O)[5- or 6-membered heteroaryl], —C(O)phenyl, —C(O)O(1-8C)alkyl, —C(O)O(3-7C)cycloalkyl, —C(O)O(CH₂)₁₋₃(3-7C)cycloalkyl, —C(O)NH₂, —C(O)NH-(1-8C)alkyl, —C(O)NH—(CH₂)₀₋₃(3-7C)cycloalkyl, —C(O)NH—(CH₂)₀₋₃heterocyclyl, —C(O)NH—(CH₂)₀₋₃[5 or 6-membered heteroaryl], —C(O)NH—(CH₂)₀₋₃phenyl, —S(O)₂H or —S(O)₂-(1-8C)alkyl; wherein any alkyl, cycloalkyl, phenyl, heteroaryl or heterocyclyl moiety is optionally substituted by one or more R₃₀₀ substituents defined herein.

Particular compounds of the invention include, for example, compounds of the Formula (I), or pharmaceutically acceptable salts, hydrates and/or solvates thereof, defined herein having one of the structural formulae (Ia), (Ib), (Ic), (Id) or (Ie) shown below:

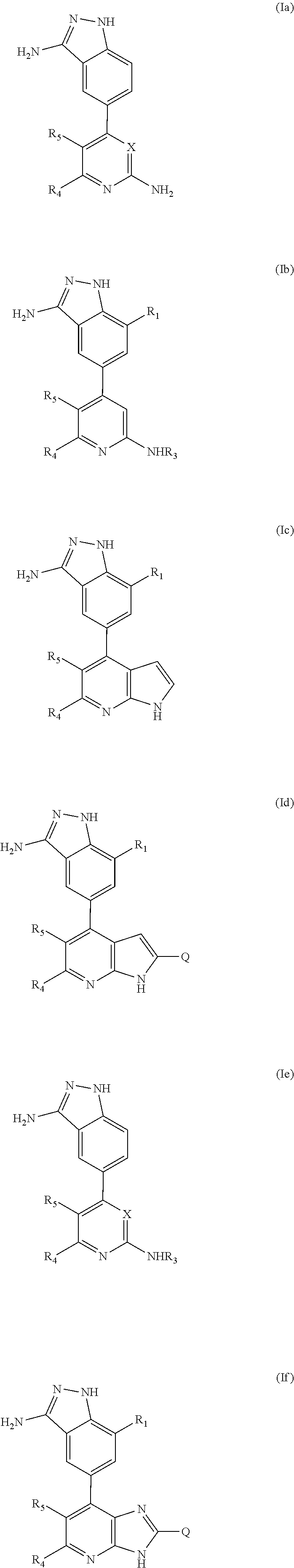

wherein R₁, X, R₃, R₄, R₅ and Q are each as defined herein.

Particular compounds of the invention include, for example, compounds of the Formula (I) [including sub-formulae (Ia), (Ib), (Ic), (Id), (Ie) or (If)], or pharmaceutically acceptable salts, hydrates and/or solvates thereof, wherein, unless otherwise stated, each of R₁, X, R₂, R₃, R₄, X₁, R₅ and Q and any associated substituent groups has any of the meanings defined hereinbefore or in any of paragraphs (1) to (43) hereinafter:
(1)Rₗ is selected from hydrogen, halogen, (1-6C)alkyl, (2-6C)alkynyl, (3-7C)cycloalkyl, phenyl, a 5 or 6-membered heteroaryl or a 4 to 7-membered heterocyclyl,
   wherein said (2-6C)alkynyl, (3-7C)cycloalkyl, phenyl, heteroaryl and heterocyclyl are optionally substituted by one or more R₁₀₀ substituents;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}heterocyclyl, (CH₂)_{z}heteroaryl, or (CH₂)_{z}phenyl;
   and wherein:
      (i) R_{f} and R_{g} are each independently selected from hydrogen or (1-2C)alkyl; and wherein Rₕ and Rⱼ are each independently selected from hydrogen or (1-2C)alkyl or Rₕ and Rⱼ together with the nitrogen atom to which they are attached form a 3-7 membered ring which may optionally include further heteroatoms; and
      (ii) any (1-4C)alkyl, (3-7C)cycloalkyl, heterocyclyl, heteroaryl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl
(2)Rₗ is selected from hydrogen, halogen, (1-6C)alkyl, (2-6C)alkynyl, phenyl or a 5 or 6-membered heteroaryl,
   wherein said (2-6C)alkynyl, phenyl or heteroaryl are optionally substituted by one or more R₁₀₀ substituents;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₙ, (CH₂)_{z}(3-7C)cycloalkyl, (CH₂)_{z}-[4- 6 membered heterocyclyl], (CH₂)_{z}-[5 or 6 membered heteroaryl] or (CH₂)_{z}phenyl;
   and wherein:
      (i) R_{f} and R_{g} are each independently selected from hydrogen or (1-2C)alkyl; and wherein Rₕ and Rⱼ are each independently selected from hydrogen or (1-2C)alkyl or Rₕ and Rⱼ together with the nitrogen atom to which they are attached form a 3-7 membered ring which may optionally include further heteroatoms; and
      (ii) any (1-4C)alkyl, (3-7C)cycloalkyl, heterocyclyl, heteroaryl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl.
(3)R₁ is selected from hydrogen, halogen, (1-6C)alkyl, (2-6C)alkynyl, phenyl or a 5 or 6-membered heteroaryl,
   wherein said (2-6C)alkynyl, phenyl or heteroaryl are optionally substituted by one or more R₁₀₀ substituents;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}-[4- 6 membered heterocyclyl], (CH₂)_{z}-[5 or 6 membered heteroaryl] or (CH₂)_{z}phenyl;
   and wherein:
      (i) R_{f} and R_{g} are each independently selected from hydrogen or (1-2C)alkyl; and
      (ii) any (1-4C)alkyl, (3-7C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;
(4)R₁ is selected from hydrogen, (2-6C)alkynyl, phenyl or a 5 or 6-membered heteroaryl,
   wherein said (2-6C)alkynyl, phenyl or heteroaryl are optionally substituted by one or more R₁₀₀ substituents;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl, or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;
(5)R₁ is selected from:
   (i) hydrogen;
   (ii) halo;
   (iii) methyl;
   (iv) CF₃;
   (v) ethynyl, i.e.

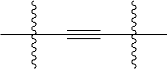 [SMILES: *C#C*]

which is optionally substituted by R₁₀₀;
(vi) phenyl, which is optionally substituted by R₁₀₀;
(vii) a 5 or 6-membered heteroaryl, which is optionally substituted by R₁₀₀;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl.
(6) R₁ is selected from:
   (i) hydrogen;
   (ii) ethynyl, i.e.

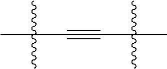 [SMILES: *C#C*]

which is optionally substituted by R₁₀₀;
(iii) phenyl, which is optionally substituted by R₁₀₀;
(iv) a 5 or 6-membered heteroaryl;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;
(7) R₁ is selected from:
   (i) hydrogen;
   (ii) ethynyl, i.e.

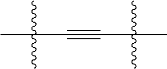 [SMILES: *C#C*]

which is optionally substituted by R₁₀₀;
(iii) phenyl, which is optionally substituted by R₁₀₀;
(iv) a 5 or 6-membered heteroaryl;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;
(8)R₁ is selected from:
   (i) hydrogen;
   (ii) ethynyl, i.e.

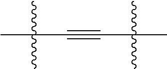 [SMILES: *C#C*]

which is optionally substituted by R₁₀₀;
(iii) phenyl, which is optionally substituted by R₁₀₀;
(iv) a 5 or 6-membered heteroaryl;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, C(O)N(Rⱼ)Rₕ, S(O)_{y}R_{f}, SO₂N(Rⱼ)Rₕ, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;
(9)R₁ is selected from:
   (i) hydrogen;
   (ii) ethynyl, i.e.

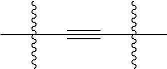 [SMILES: *C#C*]

which is optionally substituted by R₁₀₀;
(iii) phenyl, which is optionally substituted by R₁₀₀;
(iv) a 5 or 6-membered heteroaryl;
   and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, C(O)N(Rⱼ)Rₕ, S(O)_{y}R_{f}, SO₂N(Rⱼ)Rₕ, NRⱼRₕ, (CH₂)_{z}-[4-6 membered heterocyclyl], or (CH₂)_{z}phenyl;
   and wherein:
      (i) R_{f} and R_{g} are each independently selected from hydrogen or (1-2C)alkyl; and
      (ii) any (1-4C)alkyl, heterocyclyl or phenyl moiety in a R₁₀₀ substituent group is optionally further substituted by one or more substituents selected from halo, (1-2C)alkyl or ORₖ, wherein Rₖ is selected from hydrogen or methyl;
(10) X is N or CR₂; wherein R₂ is selected from hydrogen, halogen, (1-8C)alkyl, (2-8C)alkenyl, (2-8C)alkynyl, (3-7C)cycloalkyl, phenyl, a 5 or 6-membered heteroaryl or a 4 to 7-membered heterocyclyl,
   wherein said (1-6C)alkyl, (2-6C)alkenyl, (2-6C)alkynyl, (3-7C)cycloalkyl, phenyl, a 5 or 6-membered heteroaryl or a 4 to 7-membered heterocyclyl are optionally substituted by one or more R₂₀₀ substituents;
   and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl
(11) X is N or CR₂; wherein R₂ is selected from hydrogen, halogen, (1-8C)alkyl, (2-8C)alkynyl, (3-7C)cycloalkyl, phenyl, a 5 or 6-membered heteroaryl or a 4 to 7-membered heterocyclyl,
   wherein said (2-6C)alkynyl, (3-7C)cycloalkyl, phenyl, heteroaryl and heterocyclyl are optionally substituted by one or more R₂₀₀ substituents;
   and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl
(12) X is N or CR₂; wherein R₂ is selected from hydrogen, fluoro, (1-8C)alkyl, (2-8C)alkynyl, (3-7C)cycloalkyl, phenyl, or a 5 or 6-membered heteroaryl,
   wherein said (1-6C)alkynyl, (3-7C)cycloalkyl, phenyl, heteroaryl and heterocyclyl are optionally substituted by one or more R₂₀₀ substituents;
   and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl
(13) X is N or CR₂; wherein R₂ is selected from hydrogen, fluoro, (1-8C)alkyl, (3-7C)cycloalkyl, or (2-6C)alkynyl,
   wherein said (2-6C)alkynyl is optionally substituted by one or more R₂₀₀ substituents;
   and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl;
(14) X is N or CR₂; wherein R₂ is selected from:
   (i) hydrogen;
   (ii) fluoro;
   (iii) ethynyl, i.e.

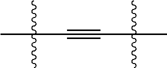 [SMILES: *C#C*]

which is optionally substituted by R₂₀₀;
(iv) phenyl, which is optionally substituted by R₂₀₀;
(v) a 5 or 6-membered heteroaryl, which is optionally substituted by R₂₀₀;
   and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
   and wherein:
      (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})R_{f}, N(R_{f})C(O)R_{q}, S(O)_{y}R_{q}, SO₂N(R_{f})R_{q}, N(R_{f})SO₂R_{q}, or NRᵣR_{q}, wherein R_{q} is hydrogen, (1-2C)alkyl or phenyl, and Rᵣ are selected from hydrogen or (1-2C)alkyl;
(15) X is N or CR₂; wherein R₂ is selected from:
   (i) hydrogen; or
   (ii) ethynyl, i.e.

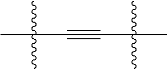 [SMILES: *C#C*]

which is optionally substituted by R₂₀₀;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
   (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
   (ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl;
(16) X is N or CR₂; wherein R₂ is selected from:
   (i) hydrogen; or
   (ii) ethynyl, i.e.

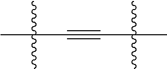 [SMILES: *C#C*]

which is optionally substituted by R₂₀₀;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
   (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-6 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
   (ii) any (3-7C)cycloalkyl, heterocyclyl, heteroaryl or phenyl moiety in a R₂₀₀ substituent group is optionally further substituted by one or more substituents selected from halo, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl;
(17) X is N or CR₂; wherein R₂ is selected from:
   (i) hydrogen; or
   (ii) ethynyl, i.e.

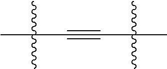 [SMILES: *C#C*]

which is optionally substituted by R₂₀₀;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
   (i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-6 membered ring; and
   (ii) any (3-7C)cycloalkyl, heterocyclyl, heteroaryl or phenyl moiety in a R₂₀₀ substituent group is optionally further substituted by one or more substituents selected from halo, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and R, are selected from hydrogen or methyl;
(18) X is CR₂ and R₂ is as defined in any one or paragraphs (10) to (17) above;
(19) R₃ is selected from hydrogen, (1-8C)alkyl, (3-7C)cycloalkyl, a carbon-linked 4 to 7 membered heterocyclyl, a carbon-linked 5 to 6 membered heteroaryl, (CH₂)₀₋₃(3-7C)cycloalkyl, —C(O)-(1-8C)alkyl, —C(O)(3-7C)cycloalkyl, —C(O)phenyl, —C(O)O(1-8C)alkyl, —C(O)NH₂, —C(O)NH-(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl;
   wherein any alkyl, cycloalkyl, phenyl, or heteroaryl moiety is optionally substituted by one or more R₃₀₀ substituents;
   wherein R₃₀₀ is selected from halo, trifluoromethyl, trifluoromethoxy, cyano, hydroxyl, (1-4C)alkyl, (1-4C)hydroxyalkyl, ORₛ, C(O)Rₛ, C(O)ORₛ, OC(O)Rₛ, C(O)N(Rᵥ)Rᵤ, N(Rₜ)C(O)Rₛ, N(Rₜ)C(O)ORₛ, S(O)_{y}Rₛ, SO₂N(Rᵥ)Rᵤ, N(Rₜ)SO₂Rₛ, (CH₂)_{z}NRᵤRᵥ;
   and wherein:
      (i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms, and wherein any 3-7 membered ring formed Rₒ and Rₚ, and any alkyl or phenyl group present for Rₛ, Rₜ, Rᵤ and Rᵥ is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;
(19a) R₃ is selected from hydrogen, cyano, (1-8C)alkyl, (3-7C)cycloalkyl, a carbon-linked 4 to 7 membered heterocyclyl, a carbon-linked 5 to 6 membered heteroaryl, (CH₂)₁₋₃(3-7C)cycloalkyl, —C(O)-(1-8C)alkyl, —C(O)(3-7C)cycloalkyl, —C(O)phenyl, —C(O)O(1-8C)alkyl, —C(O)NH₂, —C(O)NH-(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl;
   wherein any alkyl, cycloalkyl, phenyl, or heteroaryl moiety is optionally substituted by one or more R₃₀₀ substituents;
   wherein R₃₀₀ is selected from halo, trifluoromethyl, trifluoromethoxy, cyano, hydroxyl, (1-4C)alkyl, (1-4C)hydroxyalkyl, ORₛ, C(O)Rₛ, C(O)ORₛ, OC(O)Rₛ, C(O)N(Rᵥ)Rᵤ, N(Rₜ)C(O)Rₛ, N(Rₜ)C(O)ORₛ, S(O)_{y}Rₛ, SO₂N(Rᵥ)Rᵤ, N(Rₜ)SO₂Rₛ, (CH₂)_{z}NRᵤRᵥ;
   and wherein:
      (i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms, and wherein any 3-7 membered ring formed Rₒ and Rₚ, and any alkyl or phenyl group present for Rₛ, Rₜ, Rᵤ and Rᵥ is optionally further substituted by one or more substituents selected from halo, trifluoromethyl, trifluoromethoxy, cyano, hydroxyl, carboxyl, carbamoyl, sulphamoyl, and (1-2C)alkyl; and
      (ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;
(20) R₃ is selected from hydrogen, (1-8C)alkyl, (3-7C)cycloalkyl, (CH₂)₁₋₃(3-7C)cycloalkyl, a carbon-linked 4 to 7 membered heterocyclyl, a carbon-linked 5 to 6 membered heteroaryl, —C(O)-(1-8C)alkyl, —C(O)(3-7C)cycloalkyl, —C(O)phenyl, —C(O)O(1-8C)alkyl, —C(O)NH₂, —C(O)NH-(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl; wherein any alkyl, cycloalkyl, phenyl, or heteroaryl moiety is optionally substituted by one or more R₃₀₀ substituents;
   wherein R₃₀₀ is selected from halo, trifluoromethyl, trifluoromethoxy, cyano, hydroxyl, (1-4C)alkyl, (1-4C)hydroxyalkyl, ORₛ, C(O)N(Rᵥ)Rᵤ, S(O)_{y}Rₛ, (CH₂)_{z}NRᵤRᵥ;
   and wherein:
      (i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms; and
      (ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;
(20a) R₃ is selected from hydrogen, cyano, (1-8C)alkyl, (3-7C)cycloalkyl, (CH₂)₁₋₃(3-7C)cycloalkyl, a carbon-linked 4 to 7 membered heterocyclyl, a carbon-linked 5 to 6 membered heteroaryl, —C(O)-(1-8C)alkyl, —C(O)(3-7C)cycloalkyl, —C(O)phenyl, —C(O)O(1-8C)alkyl, —C(O)NH₂, —C(O)NH-(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl;
   wherein any alkyl, cycloalkyl, phenyl, or heteroaryl moiety is optionally substituted by one or more R₃₀₀ substituents;
   wherein R₃₀₀ is selected from halo, trifluoromethyl, trifluoromethoxy, cyano, hydroxyl, (1-4C)alkyl, (1-4C)hydroxyalkyl, ORₛ, C(O)N(Rᵥ)Rᵤ, S(O)_{y}Rₛ, (CH₂)_{z}NRᵤRᵥ;
   and wherein:
      (i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms; and
      (ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;
(21) R₃ is selected from hydrogen or acetyl;
(22) R₄ is selected from hydrogen or fluoro;
(23) R₄ is hydrogen;
(24) X₁ is N when R₂ and R₃ are linked such that together they form a —CH═CH— group; or CR₅ wherein R₅ is selected from hydrogen, halo or cyano;
(25) X₁ is N when R₂ and R₃ are linked such that together they form a —CH═CH— group; or CR₅ wherein R₅ is selected from hydrogen, fluoro or cyano;
(26) X₁ is N when R₂ and R₃ are linked such that together they form a —CH═CH— group; or CR₅ wherein R₅ is selected from hydrogen or cyano;
(27) X₁ is N when R₂ and R₃ are linked such that together they form a —CH═CH— group;
(28) X₁ is CR₅ wherein R₅ is selected from hydrogen, halo or cyano;
(29) X₁ is CR₅ wherein R₅ is selected from hydrogen, fluoro or cyano;
(30) X₁ is CR₅ wherein R₅ is selected from hydrogen or cyano;
(31) R₂ and R₃ are linked such that together they form a —CH═CQ- group;
(32) Q is hydrogen, or a group of the formula:

-L₁-Y₁-L₂-Q₁
wherein:
   L₁ is absent or (1-3C)alkylene;
   Y₁ is absent or O, S, SO, SO₂, N(R_{y1}), C(O), C(O)O, C(O)N(R_{y1}), or N(R_{y1})C(O), wherein R_{y1} is selected from hydrogen or (1-4C)alkyl;
   L₂ is absent or (1-3C)alkylene; and
   Q₁ is hydrogen, (1-6C)alkyl, phenyl, (3-8C)cycloalkyl, heteroaryl or heterocyclyl;
   wherein Q is optionally further substituted by one or more substituent groups independently selected from oxo, hydroxy, (1-6C)alkyl, halo, (1-4C)haloalkyl, (1-4C)haloalkoxy, (1-4C)aminoalkyl, (1-4C)hydroxyalkyl, cyano, or by one or more group(s) of the formula:

-L₃-Y₂-L₄-W₁
wherein:
   L₃ is absent;
   Y₂ is absent or selected from or O, S, SO, SO₂, N(R_{y2}), C(O), C(O)O, OC(O), C(O)N(R_{y2}), or N(R_{y2})C(O), S(O)₂N(R_{y2}), N(R_{y2})SO₂ wherein R_{y2} is selected from hydrogen or (1-3C)alkyl;
   L₄ is absent or (1-3C)alkylene; and
   W₁ is hydrogen, (1-6C)alkyl, or phenyl;
   wherein W₁ is optionally substituted by one or more substituents selected from (1-2C)alkyl, or halo;
with the proviso that Q₁ is not aryl or heteroaryl when all of L₁, Y₁ and L₂ are absent;
(33) Q is hydrogen, or a group of the formula:

-L₁-Y₁-L₂-Q₁
wherein:
   L₁ is absent or (1-3C)alkylene;
   Y₁ is absent or O, S, SO, SO₂, N(R_{y1}), C(O), C(O)O, or C(O)N(R_{y1}), wherein R_{y1} is selected from hydrogen or (1-3C)alkyl;
   L₂ is absent or (1-3C)alkylene; and
   Q₁ is hydrogen, (1-6C)alkyl, phenyl, (3-6C)cycloalkyl, 5- or 6-membered heteroaryl or 4 to 7 membered heterocyclyl;
   wherein Q is optionally further substituted by one or more substituent groups independently selected from oxo, hydroxy, (1-6C)alkyl, halo, (1-4C)haloalkyl, (1-4C)haloalkoxy, (1-4C)aminoalkyl, (1-4C)hydroxyalkyl, cyano, or by one or more group(s) of the formula:

-L₃-Y₂-L₄-W₁
wherein:
   L₃ is absent;
   Y₂ is absent or selected from or O, S, SO, SO₂, N(R_{y2}), C(O), C(O)O, OC(O), C(O)N(R_{y2}), or N(R_{y2})C(O), S(O)₂N(R_{y2}), N(R_{y2})SO₂ wherein R_{y2} is selected from hydrogen or (1-3C)alkyl;
   L₄ is absent or (1-3C)alkylene; and
   W₁ is hydrogen, (1-6C)alkyl, or phenyl;
with the proviso that Q₁ is not phenyl or heteroaryl when all of L₁, Y₁ and L₂ are absent;
(34) Q is hydrogen, or a group of the formula:

-L₁-Y₁-L₂-Q₁
wherein:
   L₁ is absent or (1-3C)alkylene;
   Y₁ is absent or O, N(R_{y1}), C(O), C(O)O, or C(O)N(R_{y1}), wherein R_{y1} is selected from hydrogen or (1-3C)alkyl;
   L₂ is absent or (1-3C)alkylene; and
   Q₁ is hydrogen, (1-6C)alkyl, phenyl, (3-6C)cycloalkyl, 5- or 6-membered heteroaryl or 4 to 7 membered heterocyclyl;
   wherein Q is optionally further substituted by one or more substituent groups independently selected from oxo, hydroxy, (1-6C)alkyl, halo, (1-4C)haloalkyl, (1-4C)haloalkoxy, cyano, or by one or more group(s) of the formula:

-L₃-Y₂-L₄-W₁
wherein:
   L₃ is absent;
   Y₂ is absent or selected from or O, N(R_{y2}), C(O), C(O)N(R_{y2}), or N(R_{y2})C(O), wherein R_{y2} is selected from hydrogen or (1-3C)alkyl;
   L₄ is absent or (1-3C)alkylene; and
   W₁ is hydrogen, (1-6C)alkyl, or phenyl;
with the proviso that Q₁ is not phenyl or heteroaryl when all of L₁, Y₁ and L₂ are absent;
(35) Q is hydrogen, or a group of the formula:

-L₁-Y₁-L₂-Q₁
wherein:
   L₁ is absent or (1-3C)alkylene;
   Y₁ is absent or O, N(R_{y1}), C(O), C(O)O, or C(O)N(R_{y1}), wherein R_{y1} is selected from hydrogen or (1-3C)alkyl;
   L₂ is absent or (1-3C)alkylene; and
   Q₁ is hydrogen, (1-6C)alkyl, phenyl, (3-6C)cycloalkyl, 5- or 6-membered heteroaryl or 4 to 7 membered heterocyclyl;
   wherein Q is optionally further substituted by one or more substituent groups independently selected from oxo, hydroxy, (1-6C)alkyl, halo, cyano, or by one or more group(s) of the formula:

-L₃-Y₂-L₄-W₁
wherein:
   L₃ is absent;
   Y₂ is absent or selected from or O, or N(R_{y2}), wherein R_{y2} is selected from hydrogen or (1-3C)alkyl;
   L₄ is absent or (1-2C)alkylene; and
   W₁ is hydrogen, (1-6C)alkyl, or phenyl;
with the proviso that Q₁ is not phenyl or heteroaryl when all of L₁, Y₁ and L₂ are absent;
(36) y is independently selected from 0, 1 or 2;
(37) y is 0;
(38) y is 1;
(39) y is 2;
(40) z is independently selected from 0, 1 or 2;
(41) z is 0;
(42) z is 1;
(43) z is 2;

Suitably, R₁ is as defined in any one of numbered paragraphs (1) to (9) above. More suitably, R₁ is as defined in any one of numbered paragraphs (5) to (9) above. Most suitably, R₁ is as defined in any one of numbered paragraphs (7) to (9) above.

Suitably, X is as defined in any one of numbered paragraphs (10) to (18) above. More suitably, X is as defined in any one of numbered paragraphs (14) to (18) above. Most suitably, X is as defined in any one of numbered paragraphs (16) to (18) above.

Suitably, R₂ is as defined in any one of numbered paragraphs (10) to (17) above, R₃ is as defined in any one of numbered paragraphs (19) to (21) above (including (19a) and (20a)), or R₂ and R₃ are linked such that together they form a —CH═CQ- group and Q is as defined any one of paragraphs (31) to (35) above. More suitably, R₂ is as defined in any one of numbered paragraphs (14) to (17) above, R₃ is as defined in any one of numbered paragraphs (20) or (21) above, or R₂ and R₃ are linked such that together they form a —CH═CQ- group and Q is as defined any one of paragraphs (33) to (35) above. Most suitably, R₂ is as defined in numbered paragraphs (16) or (17) above, R₃ is as defined in numbered paragraphs (20) or (21) above, or R₂ and R₃ are linked such that together they form a —CH═CQ- group and Q is as defined paragraphs (34) or (35) above.

Suitably, R₄ is as defined in numbered paragraph (23) above.

Suitably, X₁ is as defined in any one of numbered paragraphs (24) to (30) above. More suitably, X₁ is as defined in numbered paragraphs (28) to (30) above. Most suitably, X₁ is as defined in any one of numbered paragraphs (29) or (30) above.

Suitably, R₅ is as defined in any one of numbered paragraphs (28) to (30) above. More suitably, R₅ is as defined in numbered paragraphs (29) or (30) above. Most suitably, R₅ is as defined in numbered paragraph (30) above.

Suitably, y is as defined in numbered paragraph (36) above.

Suitably, x is as defined in numbered paragraph (40) above.

As indicated above, particular compounds of the invention include, for example, compounds of the Formula (I), or pharmaceutically acceptable salts, hydrates and/or solvates thereof, defined herein having one of the structural formulae (Ia), (Ib), (Ic), (Id) or (Ie) shown below:

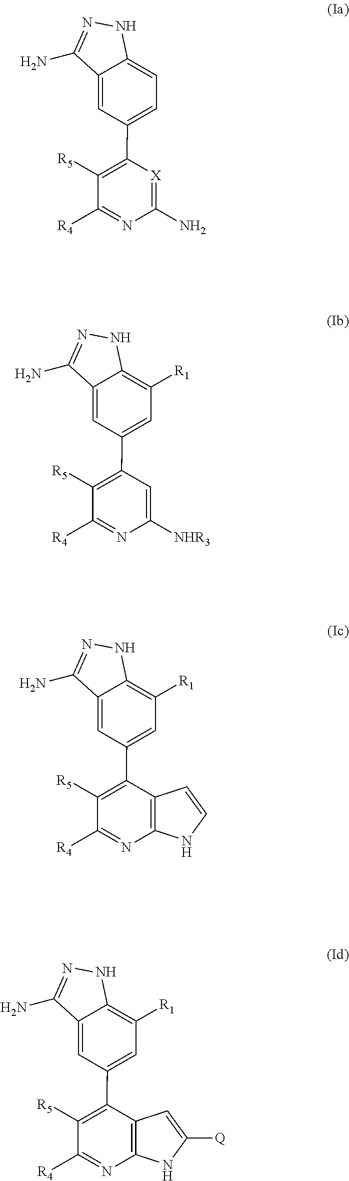

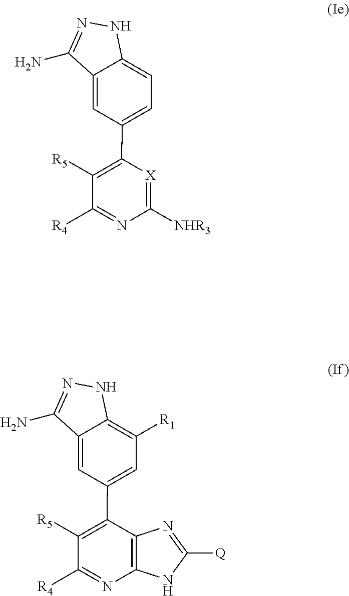

wherein R₁, X, R₃, R₄, R₅ and Q are each as defined herein.

In a particular group of compounds of the invention, compounds have a structure according to formula Ia (which is a sub-definition of formula I), or a pharmaceutically acceptable salt, hydrate and/or solvate thereof, wherein X, R₄ and R₅ each have any one of the definitions set out herein.

In an embodiment of the compounds of formula Ia, or a pharmaceutically acceptable salt, hydrate and/or solvate thereof:
X is as defined in any one of numbered paragraphs (10) to (18) above;
R₄ is as defined in numbered paragraph (23) above; and
R₅ is as defined in any one of numbered paragraphs (28) to (30) above.

In an embodiment of the compounds of formula Ia, or a pharmaceutically acceptable salt, hydrate and/or solvate thereof:
X is as defined in any one of numbered paragraphs (14) to (18) above;
R₄ is as defined in numbered paragraph (23) above; and
R₅ is as defined in numbered paragraphs (29) or (30) above.

In an embodiment of the compounds of formula Ia, or a pharmaceutically acceptable salt, hydrate and/or solvate thereof:
X is as defined in numbered paragraph (14) above;
R₄ is as defined in numbered paragraph (23) above; and
R₅ is as defined in numbered paragraph (28) above.

In an embodiment of the compounds of formula Ia, or a pharmaceutically acceptable salt, hydrate and/or solvate thereof:
X is as defined in numbered paragraph (15) above;
R₄ is as defined in numbered paragraph (23) above; and
R₅ is as defined in numbered paragraph (28) above.

In an embodiment of the compounds of formula Ia, or a pharmaceutically acceptable salt, hydrate and/or solvate thereof:
X is as defined in numbered paragraph (16) above;
R₄ is as defined in numbered paragraph (23) above; and
R₅ is as defined in numbered paragraph (29) above.

In an embodiment of the compounds of formula Ia, or a pharmaceutically acceptable salt, hydrate and/or solvate thereof:
X is as defined in numbered paragraph (17) above;
R₄ is as defined in numbered paragraph (23) above; and
R₅ is as defined in numbered paragraph (30) above.

In a particular group of compounds of the invention, compounds have a structure according to formula Ib (which is a sub-definition of formula I), or a pharmaceutically acceptable salt, hydrate and/or solvate thereof, wherein R₁, R₃, R₄, and R₅ each have any one of the definitions set out herein.

In an embodiment of the compounds of formula Ib, or a pharmaceutically acceptable salt, hydrate and/or solvate thereof:
R₁ is as defined in any one of numbered paragraphs (1) to (9) above;
R₃ is as defined in any one of numbered paragraphs (19) to (21) above (including (19a) and (20a));
R₄ is as defined in numbered paragraph (23) above; and
R₅ is as defined in any one of numbered paragraphs (28) to (30) above.

In an embodiment of the compounds of formula Ib, or a pharmaceutically acceptable salt, hydrate and/or solvate thereof:
R₁ is as defined in any one of numbered paragraphs (5) to (9) above;
R₃ is as defined in any one of numbered paragraphs (19) to (21) above (including (19a) and (20a));
R₄ is as defined in numbered paragraph (23) above; and
R₅ is as defined in numbered paragraphs (29) or (30) above.

In an embodiment of the compounds of formula Ib, or a pharmaceutically acceptable salt, hydrate and/or solvate thereof:
R₁ is as defined in numbered paragraph (5) above;
R₃ is as defined in numbered paragraph (19) above;
R₄ is as defined in numbered paragraph (23) above; and
R₅ is as defined in numbered paragraphs (28) above.

In an embodiment of the compounds of formula Ib, or a pharmaceutically acceptable salt, hydrate and/or solvate thereof:
R₁ is as defined in numbered paragraph (6) above;
R₃ is as defined in numbered paragraph (20) above;
R₄ is as defined in numbered paragraph (23) above; and
R₅ is as defined in numbered paragraphs (29) above.

In an embodiment of the compounds of formula Ib, or a pharmaceutically acceptable salt, hydrate and/or solvate thereof:
R₁ is as defined in numbered paragraph (7) above;
R₃ is as defined in numbered paragraph (20) above;
R₄ is as defined in numbered paragraph (23) above; and
R₅ is as defined in numbered paragraphs (29) above.

In an embodiment of the compounds of formula Ib, or a pharmaceutically acceptable salt, hydrate and/or solvate thereof:
R₁ is as defined in numbered paragraph (8) above;
R₃ is as defined in numbered paragraph (21) above;
R₄ is as defined in numbered paragraph (23) above; and
R₅ is as defined in numbered paragraphs (30) above.

In an embodiment of the compounds of formula Ib, or a pharmaceutically acceptable salt, hydrate and/or solvate thereof:
R₁ is as defined in numbered paragraph (9) above;
R₃ is as defined in numbered paragraph (21) above;
R₄ is as defined in numbered paragraph (23) above; and
R₅ is as defined in numbered paragraphs (30) above.

In a particular group of compounds of the invention, compounds have a structure according to formula Ic (which is a sub-definition of formula I), or a pharmaceutically acceptable salt, hydrate and/or solvate thereof, wherein R₁, R₄, and R₅ each have any one of the definitions set out herein.

In an embodiment of the compounds of formula Ic, or a pharmaceutically acceptable salt, hydrate and/or solvate thereof:
R₁ is as defined in any one of numbered paragraphs (1) to (9) above;
R₄ is as defined in numbered paragraph (23) above; and
R₅ is as defined in any one of numbered paragraphs (28) to (30) above.

In an embodiment of the compounds of formula Ic, or a pharmaceutically acceptable salt, hydrate and/or solvate thereof:
R₁ is as defined in any one of numbered paragraphs (5) to (9) above;
R₄ is as defined in numbered paragraph (23) above; and
R₅ is as defined in numbered paragraphs (29) or (30) above.

In an embodiment of the compounds of formula Ic, or a pharmaceutically acceptable salt, hydrate and/or solvate thereof:
R₁ is as defined in numbered paragraph (5) above;
R₄ is as defined in numbered paragraph (23) above; and
R₅ is as defined in numbered paragraphs (28) above.

In an embodiment of the compounds of formula Ic, or a pharmaceutically acceptable salt, hydrate and/or solvate thereof:
R₁ is as defined in numbered paragraph (6) above;
R₄ is as defined in numbered paragraph (23) above; and
R₅ is as defined in numbered paragraphs (29) above.

In an embodiment of the compounds of formula Ic, or a pharmaceutically acceptable salt, hydrate and/or solvate thereof:
R₁ is as defined in numbered paragraph (7) above;
R₄ is as defined in numbered paragraph (23) above; and
R₅ is as defined in numbered paragraphs (29) above.

In an embodiment of the compounds of formula Ic, or a pharmaceutically acceptable salt, hydrate and/or solvate thereof:
R₁ is as defined in numbered paragraph (8) above;
R₄ is as defined in numbered paragraph (23) above; and
R₅ is as defined in numbered paragraphs (30) above.

In an embodiment of the compounds of formula Ic, or a pharmaceutically acceptable salt, hydrate and/or solvate thereof:
R₁ is as defined in numbered paragraph (9) above;
R₄ is as defined in numbered paragraph (23) above; and
R₅ is as defined in numbered paragraphs (30) above.

In a particular group of compounds of the invention, compounds have a structure according to formula Id (which is a sub-definition of formula I), or a pharmaceutically acceptable salt, hydrate and/or solvate thereof, wherein R₁, Q, R₄, and R₅ each have any one of the definitions set out herein.

In an embodiment of the compounds of formula Id, or a pharmaceutically acceptable salt, hydrate and/or solvate thereof:
R₁ is as defined in any one of numbered paragraphs (1) to (9) above;
Q is as defined in any one of numbered paragraphs (32) to (35) above;
R₄ is as defined in numbered paragraph (23) above; and
R₅ is as defined in any one of numbered paragraphs (28) to (30) above.

In an embodiment of the compounds of formula Id, or a pharmaceutically acceptable salt, hydrate and/or solvate thereof:
R₁ is as defined in any one of numbered paragraphs (5) to (9) above;
Q is as defined in any one of numbered paragraphs (33) to (35) above;
R₄ is as defined in numbered paragraph (23) above; and
R₅ is as defined in numbered paragraphs (29) or (30) above.

In an embodiment of the compounds of formula Id, or a pharmaceutically acceptable salt, hydrate and/or solvate thereof:
R₁ is as defined in numbered paragraph (5) above;
Q is as defined in numbered paragraph (32) above;
R₄ is as defined in numbered paragraph (23) above; and
R₅ is as defined in numbered paragraphs (28) above.

In an embodiment of the compounds of formula Id, or a pharmaceutically acceptable salt, hydrate and/or solvate thereof:
R₁ is as defined in numbered paragraph (6) above;
Q is as defined in numbered paragraph (33) above;
R₄ is as defined in numbered paragraph (23) above; and
R₅ is as defined in numbered paragraphs (29) above.

In an embodiment of the compounds of formula Id, or a pharmaceutically acceptable salt, hydrate and/or solvate thereof:
R₁ is as defined in numbered paragraph (7) above;
Q is as defined in numbered paragraph (33) above;
R₄ is as defined in numbered paragraph (23) above; and
R₅ is as defined in numbered paragraphs (29) above.

In an embodiment of the compounds of formula Id, or a pharmaceutically acceptable salt, hydrate and/or solvate thereof:
R₁ is as defined in numbered paragraph (8) above;
Q is as defined in numbered paragraph (34) above;
R₄ is as defined in numbered paragraph (23) above; and
R₅ is as defined in numbered paragraphs (30) above.

In an embodiment of the compounds of formula Id, or a pharmaceutically acceptable salt, hydrate and/or solvate thereof:
R₁ is as defined in numbered paragraph (9) above;
Q is as defined in numbered paragraph (35) above;
R₄ is as defined in numbered paragraph (23) above; and
R₅ is as defined in numbered paragraphs (30) above.

In a particular group of compounds of the invention, compounds have a structure according to formula Ie (which is a sub-definition of formula I), or a pharmaceutically acceptable salt, hydrate and/or solvate thereof, wherein X, R₃, R₄ and R₅ each have any one of the definitions set out herein.

In an embodiment of the compounds of formula Ie, or a pharmaceutically acceptable salt, hydrate and/or solvate thereof:
X is as defined in any one of numbered paragraphs (10) to (18) above;
R₃ is as defined in any one of numbered paragraphs (19) to (21) above (including (19a) and (20a));
R₄ is as defined in numbered paragraph (23) above; and
R₅ is as defined in any one of numbered paragraphs (28) to (30) above.

In an embodiment of the compounds of formula Ie, or a pharmaceutically acceptable salt, hydrate and/or solvate thereof:
X is as defined in any one of numbered paragraphs (14) to (18) above;
R₃ is as defined in any one of numbered paragraphs (19) to (21) above (including (19a) and (20a));
R₄ is as defined in numbered paragraph (23) above; and
R₅ is as defined in numbered paragraphs (29) or (30) above.

In an embodiment of the compounds of formula Ie, or a pharmaceutically acceptable salt, hydrate and/or solvate thereof:
X is as defined in numbered paragraph (14) above;
R₃ is as defined in numbered paragraph (19) above;
R₄ is as defined in numbered paragraph (23) above; and
R₅ is as defined in numbered paragraphs (28) above.

In an embodiment of the compounds of formula Ie, or a pharmaceutically acceptable salt, hydrate and/or solvate thereof:
X is as defined in numbered paragraph (14) above;
R₃ is as defined in numbered paragraph (20) above;
R₄ is as defined in numbered paragraph (23) above; and
R₅ is as defined in numbered paragraphs (29) above.

In an embodiment of the compounds of formula Ie, or a pharmaceutically acceptable salt, hydrate and/or solvate thereof:
X is as defined in numbered paragraph (15) above;
R₃ is as defined in numbered paragraph (20) above;
R₄ is as defined in numbered paragraph (23) above; and
R₅ is as defined in numbered paragraphs (29) above.

In an embodiment of the compounds of formula Ie, or a pharmaceutically acceptable salt, hydrate and/or solvate thereof:
X is as defined in numbered paragraph (16) above;
R₃ is as defined in numbered paragraph (21) above;
R₄ is as defined in numbered paragraph (23) above; and
R₅ is as defined in numbered paragraphs (30) above.

In an embodiment of the compounds of formula Ie, or a pharmaceutically acceptable salt, hydrate and/or solvate thereof:
X is as defined in numbered paragraph (17) above;
R₃ is as defined in numbered paragraph (21) above;
R₄ is as defined in numbered paragraph (23) above; and
R₅ is as defined in numbered paragraphs (30) above.

In a particular group of compounds of the invention, compounds have a structure according to formula If (which is a sub-definition of formula I), or a pharmaceutically acceptable salt, hydrate and/or solvate thereof, wherein R₁, Q, R₄, and R₅ each have any one of the definitions set out herein.

In an embodiment of the compounds of formula If, or a pharmaceutically acceptable salt, hydrate and/or solvate thereof:
R₁ is as defined in any one of numbered paragraphs (1) to (9) above;
Q is as defined in any one of numbered paragraphs (32) to (35) above;
R₄ is as defined in numbered paragraph (23) above; and
R₅ is as defined in any one of numbered paragraphs (28) to (30) above.

In an embodiment of the compounds of formula If, or a pharmaceutically acceptable salt, hydrate and/or solvate thereof:
R₁ is as defined in any one of numbered paragraphs (5) to (9) above;
Q is as defined in any one of numbered paragraphs (33) to (35) above;
R₄ is as defined in numbered paragraph (23) above; and
R₅ is as defined in numbered paragraphs (29) or (30) above.

In an embodiment of the compounds of formula If, or a pharmaceutically acceptable salt, hydrate and/or solvate thereof:
R₁ is as defined in numbered paragraph (5) above;
Q is as defined in numbered paragraph (32) above;
R₄ is as defined in numbered paragraph (23) above; and
R₅ is as defined in numbered paragraphs (28) above.

In an embodiment of the compounds of formula If, or a pharmaceutically acceptable salt, hydrate and/or solvate thereof:
R₁ is as defined in numbered paragraph (6) above;
Q is as defined in numbered paragraph (33) above;
R₄ is as defined in numbered paragraph (23) above; and
R₅ is as defined in numbered paragraphs (29) above.

In an embodiment of the compounds of formula If, or a pharmaceutically acceptable salt, hydrate and/or solvate thereof:
R₁ is as defined in numbered paragraph (7) above;
Q is as defined in numbered paragraph (33) above;
R₄ is as defined in numbered paragraph (23) above; and
R₅ is as defined in numbered paragraphs (29) above.

In an embodiment of the compounds of formula If, or a pharmaceutically acceptable salt, hydrate and/or solvate thereof:
R₁ is as defined in numbered paragraph (8) above;
Q is as defined in numbered paragraph (34) above;
R₄ is as defined in numbered paragraph (23) above; and
R₅ is as defined in numbered paragraphs (30) above.

In an embodiment of the compounds of formula If, or a pharmaceutically acceptable salt, hydrate and/or solvate thereof:
R₁ is as defined in numbered paragraph (9) above;
Q is as defined in numbered paragraph (35) above;
R₄ is as defined in numbered paragraph (23) above; and
R₅ is as defined in numbered paragraphs (30) above.

Particular compounds of the present invention include any of the compounds exemplified in the present application, or a pharmaceutically acceptable salt or solvate thereof, and, in particular, any of the following:
5-(2-aminopyridin-4-yl)-7-chloro-1H-indazol-3-amine;
5-(2-aminopyridin-4-yl)-7-methyl-1H-indazol-3-amine;
5-(2-aminopyridin-4-yl)-7-(trifluoromethyl)-1H-indazol-3-amine;
7-chloro-5-(1H-pyrrolo[2,3-b]pyridin-4-yl)-1H-indazol-3-amine;
7-bromo-5-(1H-pyrrolo[2,3-b]pyridin-4-yl)-1H-indazol-3-amine;
7-ethynyl-5-(1H-pyrrolo[2,3-b]pyridin-4-yl)-1H-indazol-3-amine;
7-phenyl-5-(1H-pyrrolo[2,3-b]pyridin-4-yl)-1H-indazol-3-amine;
5-(2-methyl-1H-pyrrolo[2,3-b]pyridin-4-yl)-1H-indazol-3-amine;
5-(2-(tert-butyl)-1H-pyrrolo[2,3-b]pyridin-4-yl)-1H-indazol-3-amine;
4-(3-amino-1H-indazol-5-yl)-1H-pyrrolo[2,3-b]pyridine-2-carboxylic acid;
7-bromo-5-(7H-pyrrolo[2,3-d]pyrimidin-4-yl)-1H-indazol-3-amine
5-(2-(ethylamino)pyridin-4-yl)-1H-indazol-3-amine;
5-(2-(propylamino)pyridin-4-yl)-1H-indazol-3-amine;
5-(2-(isopropylamino)pyridin-4-yl)-1H-indazol-3-amine;
5-(2-((cyclopropylmethyl)amino)pyridin-4-yl)-1H-indazol-3-amine;
5-(2-(isopentylamino)pyridin-4-yl)-1H-indazol-3-amine;
5-(2-(hexylamino)pyridin-4-yl)-1H-indazol-3-amine;
5-(2-(cyclohexylamino)pyridin-4-yl)-1H-indazol-3-amine;
5-{2-[(Trans-4-methylcyclohexyl)amino]pyridin-4-yl}-1H-indazol-3-amine;
2-((4-(3-amino-1H-indazol-5-yl)pyridin-2-yl)amino)ethan-1-ol;
3-((4-(3-amino-1H-indazol-5-yl)pyridin-2-yl)amino)propan-1-ol;
4-((4-(3-amino-1H-indazol-5-yl)pyridin-2-yl)amino)butan-1-ol;
5-((4-(3-amino-1H-indazol-5-yl)pyridin-2-yl)amino)pentan-1-ol;
5-{2-[(trans-4-hydroxycyclohexyl)amino]pyridin-4-yl}-1H-indazol-3-amine;
5-(2-((2-methoxyethyl)amino)pyridin-4-yl)-1H-indazol-3-amine;
5-(2-((3-methoxypropyl)amino)pyridin-4-yl)-1H-indazol-3-amine;
5-(2-((3-isopropoxypropyl)amino)pyridin-4-yl)-1H-indazol-3-amine;
3-((4-(3-amino-1H-indazol-5-yl)pyrimidin-2-yl)amino)propan-1-ol;
3-((4-(3-amino-1H-indazol-5-yl)pyridin-2-yl)(methyl)amino)propan-1-ol;
5-(2-((2-morpholinoethyl)amino)pyridin-4-yl)-1H-indazol-3-amine;
5-(2-((2-(piperidin-1-yl)ethyl)amino)pyridin-4-yl)-1H-indazol-3-amine;
N¹-(4-(3-amino-1H-indazol-5-yl)pyridin-2-yl)-N3-methylpropane-1,3-diamine;
N-(4-(3-amino-1H-indazol-5-yl)pyridin-2-yl)cyclopropanecarboxamide;
N-(4-(3-amino-1H-indazol-5-yl)pyridin-2-yl)benzamide;
ethyl (4-(3-amino-1H-indazol-5-yl)pyridine-2-yl)carbamate;
1-(4-(3-amino-1H-indazol-5-yl)pyridine-2-yl)-3-ethylurea;
1-(4-(3-amino-1H-indazol-5-yl)pyridine-2-yl)-3-ethylurea;
1-(4-(3-amino-1H-indazol-5-yl)pyridine-2-yl)-3-propylurea;
1-(4-(3-amino-1H-indazol-5-yl)pyridine-2-yl)-3-isopentylurea;
1-(4-(3-amino-1H-indazol-5-yl)pyridine-2-yl)-3-cyclopentylurea;
1-(4-(3-amino-1H-indazol-5-yl)pyridine-2-yl)-3-cyclohexylurea;
1-(4-(3-amino-1H-indazol-5-yl)pyridine-2-yl)-3-(2-hydroxyethyl)urea;
1-(4-(3-amino-1H-indazol-5-yl)pyridine-2-yl)-3-(3-hydroxypropyl)urea;
1-(4-(3-amino-1H-indazol-5-yl)pyridine-2-yl)-3-(2-methoxyethyl)urea;
3-(4-(3-amino-1H-indazol-5-yl)pyridine-2-yl)-1-(2-hydroxyethyl)-1-methylurea;
1-(4-(3-amino-1H-indazol-5-yl)pyridine-2-yl)-3-benzylurea;
1-(4-(3-amino-1H-indazol-5-yl)pyridine-2-yl)-3-phenethylurea;
1-(4-(3-amino-1H-indazol-5-yl)pyridine-2-yl)-3-(pyridine-2-ylmethyl)urea;
1-(4-(3-amino-1H-indazol-5-yl)pyridine-2-yl)-3-(pyridine-3-ylmethyl)urea;
1-(4-(3-amino-1H-indazol-5-yl)pyridine-2-yl)-3-(pyridine-4-ylmethyl)urea;
1-(4-(3-amino-1H-indazol-5-yl)pyridine-2-yl)-3-phenylurea;
1-(4-(3-amino-1H-indazol-5-yl)pyridine-2-yl)-3-(3-fluorophenyl)urea;
1-(4-(3-amino-1H-indazol-5-yl)pyridine-2-yl)-3-(3-chlorophenyl)urea;
1-(4-(3-amino-1H-indazol-5-yl)pyridine-2-yl)-3-(3-isopropylphenyl)urea;
1-(4-(3-amino-1H-indazol-5-yl)pyridine-2-yl)-3-(3-(hydroxymethyl)phenyl)urea;
3-(3-(4-(3-amino-1H-indazol-5-yl)pyridine-2-yl)ureido)benzamide;
1-(4-(3-amino-1H-indazol-5-yl)pyridine-2-yl)-3-(3-phenoxyphenyl)urea;
1-(4-(3-amino-1H-indazol-5-yl)pyridine-2-yl)-3-(3-(benzyloxy)phenyl)urea;
1-(4-(3-amino-1H-indazol-5-yl)pyridine-2-yl)-3-(3-((4-fluorobenzyl)oxy)phenyl)urea;
1-(4-(3-amino-1H-indazol-5-yl)pyridine-2-yl)-3-(3-((3-fluorobenzyl)oxy)phenyl)urea;
1-(4-(3-amino-1H-indazol-5-yl)pyridine-2-yl)-3-(3-((2-fluorobenzyl)oxy)phenyl)urea;
3-(3-(4-(3-amino-1H-indazol-5-yl)pyridine-2-yl)ureido)-N-phenylbenzamide;
1-(4-(3-amino-1H-indazol-5-yl)pyridine-2-yl)-3-(4-fluorophenyl)urea;
1-(4-(3-amino-1H-indazol-5-yl)pyridine-2-yl)-3-(4-chlorophenyl)urea;
1-(4-(3-amino-1H-indazol-5-yl)pyridine-2-yl)-3-(4-(tert-butyl)phenyl)urea;
1-(4-(3-amino-1H-indazol-5-yl)pyridine-2-yl)-3-(4-(methylsulfonyl)phenyl)urea;
1-(4-(3-amino-1H-indazol-5-yl)pyridin-2-yl)-3-(o-tolyl)urea;
1-(4-(3-amino-1H-indazol-5-yl)pyridine-2-yl)-3-(2-ethylphenyl)urea;
1-(4-(3-amino-1H-indazol-5-yl)pyridine-2-yl)-3-(2-isopropylphenyl)urea;
1-(4-(3-amino-1H-indazol-5-yl)pyridine-2-yl)-3-(pyridine-3-yl)urea;
5-(2-amino-3-ethynylpyridin-4-yl)-1H-indazol-3-amine;
5-(2-amino-3-(cyclopropylethynyl)pyridin-4-yl)-1H-indazol-3-amine;
5-(2-amino-3-(3,3-dimethylbut-1-yn-1-yl)pyridine-4-yl)-1H-indazol-3-amine;
5-(2-amino-3-(cyclopentylethynyl)pyridine-4-yl)-1H-indazol-3-amine;
5-(2-amino-3-(cyclohexylethynyl)pyridine-4-yl)-1H-indazol-3-amine;
5-(2-amino-3-(phenylethynyl)pyridine-4-yl)-1H-indazol-3-amine;
5-(2-amino-3-((4-aminophenyl)ethynyl)pyridine-4-yl)-1H-indazol-3-amine;
5-(2-amino-3-((3-aminophenyl)ethynyl)pyridine-4-yl)-1H-indazol-3-amine;
5-(2-amino-3-((2-aminophenyl)ethynyl)pyridine-4-yl)-1H-indazol-3-amine;
methyl 3-((2-amino-4-(3-amino-1H-indazol-5-yl)pyridine-3-yl)ethynyl)benzoate;
methyl 4-((2-amino-4-(3-amino-1H-indazol-5-yl)pyridine-3-yl)ethynyl)benzoate;
5-(2-amino-3-((2-methoxyphenyl)ethynyl)pyridine-4-yl)-1H-indazol-3-amine;
5-(2-amino-4-(3-amino-1H-indazol-5-yl)pyridine-3-yl)-1-phenylpent-4-yn-1-one;
3-(2-amino-4-(3-amino-1H-indazol-5-yl)pyridine-3-yl)prop-2-yn-1-ol;
4-(2-amino-4-(3-amino-1H-indazol-5-yl)pyridine-3-yl)but-3-yn-1-ol;
5-(2-amino-4-(3-amino-1H-indazol-5-yl)pyridine-3-yl)pent-4-yn-1-ol;
6-(2-amino-4-(3-amino-1H-indazol-5-yl)pyridine-3-yl)-2-methylhex-5-yn-2-ol;
4-(2-amino-4-(3-amino-1H-indazol-5-yl)pyridine-3-yl)-2-methylbut-3-yn-2-ol;
5-(2-amino-3-(3-(tert-butoxy)prop-1-yn-1-yl)pyridine-4-yl)-1H-indazol-3-amine;
1-((2-amino-4-(3-amino-1H-indazol-5-yl)pyridine-3-yl)ethynyl)cyclopentan-1-ol;
1-((2-amino-4-(3-amino-1H-indazol-5-yl)pyridine-3-yl)ethynyl)cyclohexan-1-ol;
1-((2-amino-4-(3-amino-1H-indazol-5-yl)pyridine-3-yl)ethynyl)cycloheptan-1-ol;
5-(2-amino-3-(3-amino-3-methylbut-1-yn-1-yl)pyridin-4-yl)-1H-indazol-3-amine;
5-(2-amino-3-(4-(piperidin-1-yl)but-1-yn-1-yl)pyridin-4-yl)-1H-indazol-3-amine;
5-(2-amino-3-(4-morpholinobut-1-yn-1-yl)pyridin-4-yl)-1H-indazol-3-amine;
5-(2-amino-3-(5-(piperidin-1-yl)pent-1-yn-1-yl)pyridin-4-yl)-1H-indazol-3-amine;
5-(2-amino-3-(5-morpholinopent-1-yn-1-yl)pyridin-4-yl)-1H-indazol-3-amine;
5-(2-amino-3-(3-(piperidin-4-yl)prop-1-yn-1-yl)pyridin-4-yl)-1H-indazol-3-amine;
3-(2-amino-4-(3-amino-1H-indazol-5-yl)pyridin-3-yl)-N-methylpropiolamide;
5-(2-amino-4-(3-amino-1H-indazol-5-yl)pyridine-3-yl)-1-morpholinopent-4-yn-1-one;
5-(2-amino-3-cyclopropylpyridin-4-yl)-1H-indazol-3-amine;
4-(2-amino-4-(3-amino-1H-indazol-5-yl)pyridin-3-yl)butan-1-ol;
1-(2-(2-amino-4-(3-amino-1H-indazol-5-yl)pyridin-3-yl)ethyl)cyclohexan-1-ol;
5-(2-amino-4-(3-amino-1H-indazol-5-yl)pyridin-3-yl)pentan-1-ol;
5-(2-aminopyridin-4-yl)-7-phenyl-1H-indazol-3-amine;
5-(2-aminopyridin-4-yl)-7-(3-fluorophenyl)-1H-indazol-3-amine;
5-(2-aminopyridin-4-yl)-7-(3-(trifluoromethyl)phenyl)-1H-indazol-3-amine;
7-(3-aminophenyl)-5-(2-aminopyridin-4-yl)-1H-indazol-3-amine;
3-(3-amino-5-(2-aminopyridin-4-yl)-1H-indazol-7-yl) phenol;
5-(2-aminopyridin-4-yl)-7-(3-methoxyphenyl)-1H-indazol-3-amine;
(3-(3-amino-5-(2-aminopyridin-4-yl)-1H-indazol-7-yl)phenyl)methanol;
3-(3-amino-5-(2-aminopyridin-4-yl)-1H-indazol-7-yl)benzaldehyde;
ethyl 3-(3-amino-5-(2-aminopyridin-4-yl)-1H-indazol-7-yl)benzoate;
3-(3-amino-5-(2-aminopyridin-4-yl)-1H-indazol-7-yl)benzamide;
3-(3-amino-5-(2-aminopyridin-4-yl)-1H-indazol-7-yl)benzenesulfonamide;
5-(2-aminopyridin-4-yl)-7-(3-(methylsulfonyl)phenyl)-1H-indazol-3-amine;
5-(2-aminopyridin-4-yl)-7-(3-(morpholinomethyl)phenyl)-1H-indazol-3-amine;
4-(3-amino-5-(2-aminopyridin-4-yl)-1H-indazol-7-yl) phenol;
(4-(3-amino-5-(2-aminopyridin-4-yl)-1H-indazol-7-yl)phenyl)methanol;
5-(2-aminopyridin-4-yl)-7-(4-(dimethylamino)phenyl)-1H-indazol-3-amine;
4-(3-amino-5-(2-aminopyridin-4-yl)-1H-indazol-7-yl)benzamide;
4-(3-amino-5-(2-aminopyridin-4-yl)-1H-indazol-7-yl)benzenesulfonamide;
5-(2-aminopyridin-4-yl)-7-(4-(morpholinomethyl)phenyl)-1H-indazol-3-amine;
5-(2-aminopyridin-4-yl)-7-(4-(tert-butyl)phenyl)-1H-indazol-3-amine;
5-(2-aminopyridin-4-yl)-7-(2-chlorophenyl)-1H-indazol-3-amine;
(2-(3-amino-5-(2-aminopyridin-4-yl)-1H-indazol-7-yl)phenyl)methanol;
4-(3-amino-5-(2-aminopyridin-4-yl)-1H-indazol-7-yl)-3-methylbenzenesulfonamide;
5-(2-aminopyridin-4-yl)-7-(pyridin-3-yl)-1H-indazol-3-amine;
5-(2-aminopyridin-4-yl)-7-(pyridin-4-yl)-1H-indazol-3-amine;
5-(2-aminopyridin-4-yl)-7-(furan-3-yl)-1H-indazol-3-amine;
5-(2-aminopyridin-4-yl)-7-(thiophen-3-yl)-1H-indazol-3-amine;
5-(2-aminopyridin-4-yl)-7-(thiophen-2-yl)-1H-indazol-3-amine;
5-(2-aminopyridin-4-yl)-7-(thiazol-5-yl)-1H-indazol-3-amine;
5-(2-aminopyridin-4-yl)-7-(1H-pyrazol-5-yl)-1H-indazol-3-amine;
5-(2-aminopyridin-4-yl)-7-(3-methylbut-1-yn-1-yl)-1H-indazol-3-amine;
5-(2-aminopyridin-4-yl)-7-(pent-1-yn-1-yl)-1H-indazol-3-amine;
5-(2-aminopyridin-4-yl)-7-(cyclopropylethynyl)-1H-indazol-3-amine;
5-(2-aminopyridin-4-yl)-7-(3,3-dimethylbut-1-yn-1-yl)-1H-indazol-3-amine;
5-(2-aminopyridin-4-yl)-7-(phenylethynyl)-1H-indazol-3-amine;
4-(3-amino-5-(2-aminopyridin-4-yl)-1H-indazol-7-yl)but-3-yn-1-ol;
4-(3-amino-5-(2-aminopyridin-4-yl)-1H-indazol-7-yl)-2-methylbut-3-yn-2-ol;
5-(2-aminopyridin-4-yl)-7-((3-methyloxetan-3-yl)ethynyl)-1H-indazol-3-amine;
5-(2-aminopyridin-4-yl)-7-((tetrahydro-2H-pyran-4-yl)ethynyl)-1H-indazol-3-amine;
5-(2-aminopyrimidin-4-yl)-7-(3,3-dimethylbut-1-yn-1-yl)-1H-indazol-3-amine;
5-(2-aminopyridin-4-yl)-7-(3,3-dimethylbutyl)-1H-indazol-3-amine;
5-(2-aminopyridin-4-yl)-7-(2-cyclohexylethyl)-1H-indazol-3-amine;
5-(2-aminopyridin-4-yl)-7-(2-cyclopropylethyl)-1H-indazol-3-amine;
5-(2-aminopyridin-4-yl)-7-phenethyl-1H-indazol-3-amine;
5-(2-amino-5-fluoropyridin-4-yl)-7-(3,3-dimethylbut-1-yn-1-yl)-1H-indazol-3-amine;
5-(2-amino-3-fluoropyridin-4-yl)-7-(3,3-dimethylbut-1-yn-1-yl)-1H-indazol-3-amine;
5-(2-amino-6-fluoropyridin-4-yl)-7-(3,3-dimethylbut-1-yn-1-yl)-1H-indazol-3-amine;
4-(3-amino-7-(3,3-dimethylbut-1-yn-1-yl)-1H-indazol-5-yl)pyridine-2,6-diamine;
6-amino-4-(3-amino-7-(3,3-dimethylbut-1-yn-1-yl)-1H-indazol-5-yl)nicotinonitrile;
5-(2-(cyclopropylamino)pyridine-4-yl)-7-(3,3-dimethylbut-1-yn-1-yl)-1H-indazol-3-amine;
5-(2-(cyclobutylamino)pyridin-4-yl)-7-(3,3-dimethylbut-1-yn-1-yl)-1H-indazol-3-amine;
7-(3,3-dimethylbut-1-yn-1-yl)-5-(2-(oxetan-3-ylamino)pyridin-4-yl)-1H-indazol-3-amine;
5-(2-(cyclopentylamino)pyridine-4-yl)-7-(3,3-dimethylbut-1-yn-1-yl)-1H-indazol-3-amine;
5-(2-((cyclopropylmethyl)amino)pyridin-4-yl)-7-(3,3-dimethylbut-1-yn-1-yl)-1H-indazol-3-amine;
7-(3,3-dimethylbut-1-yn-1-yl)-5-(2-((2,2,2-trifluoroethyl)amino)pyridin-4-yl)-1H-indazol-3-amine;
3-((4-(3-amino-7-(3,3-dimethylbut-1-yn-1-yl)-1H-indazol-5-yl)pyridin-2-yl)amino)propanenitrile;
2-((4-(3-amino-7-(3,3-dimethylbut-1-yn-1-yl)-1H-indazol-5-yl)pyridin-2-yl)amino)ethan-1-ol;
N1-(4-(3-amino-7-(3,3-dimethylbut-1-yn-1-yl)-1H-indazol-5-yl)pyridin-2-yl)ethane-1,2-diamine;
7-(3,3-dimethylbut-1-yn-1-yl)-5-(2-((2-methoxyethyl)amino)pyridin-4-yl)-1H-indazol-3-amine;
7-(3,3-dimethylbut-1-yn-1-yl)-5-(2-((3-methoxypropyl)amino)pyridine-4-yl)-1H-indazol-3-amine;
N-(4-(3-amino-7-(3,3-dimethylbut-1-yn-1-yl)-1H-indazol-5-yl)pyridin-2-yl)acetamide;
N-(4-(3-amino-7-(3,3-dimethylbut-1-yn-1-yl)-1H-indazol-5-yl)pyridin-2-yl)propionamide;
N-(4-(3-amino-7-(3,3-dimethylbut-1-yn-1-yl)-1H-indazol-5-yl)pyridin-2-yl)-3,3,3-trifluoropropanamide;
N-(4-(3-amino-7-(3,3-dimethylbut-1-yn-1-yl)-1H-indazol-5-yl)pyridin-2-yl)cyclopropanecarboxamide;
N-(4-(3-amino-7-(3,3-dimethylbut-1-yn-1-yl)-1H-indazol-5-yl)pyridin-2-yl)isobutyramide;
N-(4-(3-amino-7-(3,3-dimethylbut-1-yn-1-yl)-1H-indazol-5-yl)pyridin-2-yl)pivalamide;
N-(4-(3-amino-7-(3,3-dimethylbut-1-yn-1-yl)-1H-indazol-5-yl)pyridin-2-yl)-2-cyclopropylacetamide;
N-(4-(3-amino-7-(3,3-dimethylbut-1-yn-1-yl)-1H-indazol-5-yl)pyridine-2-yl)-3-methylbutanamide
N-(4-(3-amino-7-(3,3-dimethylbut-1-yn-1-yl)-1H-indazol-5-yl)pyridin-2-yl)cyclobutanecarboxamide;
N-(4-(3-amino-7-(3,3-dimethylbut-1-yn-1-yl)-1H-indazol-5-yl)pyridin-2-yl)cyclopentanecarboxamide;
N-(4-(3-amino-7-(3,3-dimethylbut-1-yn-1-yl)-1H-indazol-5-yl)pyridin-2-yl)-2-hydroxyacetamide;
N-(4-(3-amino-7-(3,3-dimethylbut-1-yn-1-yl)-1H-indazol-5-yl)pyridin-2-yl)-2-methoxyacetamide;
5-(2-(cyclopropylamino)pyridine-4-yl)-7-((3-methyloxetan-3-yl)ethynyl)-1H-indazol-3-amine;
N-(4-(3-amino-7-((3-methyloxetan-3-yl)ethynyl)-1H-indazol-5-yl)pyridin-2-yl)acetamide;
N-(4-(3-amino-7-(phenylethynyl)-1H-indazol-5-yl)pyridin-2-yl)acetamide;
methyl (4-(3-amino-7-(cyclopropylethynyl)-1H-indazol-5-yl)pyridin-2-yl)carbamate;
methyl (4-(3-amino-7-(3-hydroxy-3-methylbut-1-yn-1-yl)-1H-indazol-5-yl)pyridin-2-yl)carbamate
methyl (4-(3-amino-7-(3-amino-3-methylbut-1-yn-1-yl)-1H-indazol-5-yl)pyridin-2-yl)carbamate;
methyl (4-(3-amino-7-(3-methoxy-3-methylbut-1-yn-1-yl)-1H-indazol-5-yl)pyridin-2-yl)carbamate;
methyl (4-(3-amino-7-(3-morpholinoprop-1-yn-1-yl)-1H-indazol-5-yl)pyridin-2-yl)carbamate;
Methyl (4-(3-amino-7-(4-morpholinobut-1-yn-1-yl)-1H-indazol-5-yl)pyridin-2-yl)carbamate;
1-(4-(3-amino-7-(3,3-dimethylbut-1-yn-1-yl)-1H-indazol-5-yl)pyridin-2-yl)urea;
1-(4-(3-amino-7-(3,3-dimethylbut-1-yn-1-yl)-1H-indazol-5-yl)pyridin-2-yl)-3-methylurea;
1-(4-(3-amino-7-(3,3-dimethylbut-1-yn-1-yl)-1H-indazol-5-yl)pyridin-2-yl)-3-ethylurea;
1-(4-(3-amino-7-(3,3-dimethylbut-1-yn-1-yl)-1H-indazol-5-yl)pyridin-2-yl)-3-propylurea;
1-(4-(3-amino-7-(3,3-dimethylbut-1-yn-1-yl)-1H-indazol-5-yl)pyridine-2-yl)-3-phenylurea;
methyl (4-(3-amino-7-(3,3-dimethylbut-1-yn-1-yl)-1H-indazol-5-yl)pyridin-2-yl)carbamate;
ethyl (4-(3-amino-7-(3,3-dimethylbut-1-yn-1-yl)-1H-indazol-5-yl)pyridin-2-yl)carbamate;
tert-butyl (4-(3-amino-7-(3,3-dimethylbut-1-yn-1-yl)-1H-indazol-5-yl)pyridin-2-yl)carbamate;
(4-(3-amino-7-(3,3-dimethylbut-1-yn-1-yl)-1H-indazol-5-yl)pyridin-2-yl)sulfamic acid;
N-(4-(3-amino-7-(3,3-dimethylbut-1-yn-1-yl)-1H-indazol-5-yl)pyridin-2-yl)methanesulfonamide;
N-(4-(3-amino-7-(3,3-dimethylbut-1-yn-1-yl)-1H-indazol-5-yl)-6-fluoropyridin-2-yl)acetamide;
N-(4-(3-amino-7-phenyl-1H-indazol-5-yl)pyridin-2-yl)acetamide;
N-(4-(3-amino-7-(pyridin-4-yl)-1H-indazol-5-yl)pyridin-2-yl)acetamide;
7-(furan-3-yl)-5-(1H-pyrrolo[2,3-b]pyridin-4-yl)-1H-indazol-3-amine;
7-ethynyl-5-(1H-pyrrolo[2,3-b]pyridin-4-yl)-1H-indazol-3-amine;
7-(3,3-dimethylbut-1-yn-1-yl)-5-(1H-pyrrolo[2,3-b]pyridin-4-yl)-1H-indazol-3-amine;
7-(cyclopropylethynyl)-5-(1H-pyrrolo[2,3-b]pyridin-4-yl)-1H-indazol-3-amine;
7-(cyclopentylethynyl)-5-(1H-pyrrolo[2,3-b]pyridin-4-yl)-1H-indazol-3-amine;
7-(cyclohexylethynyl)-5-(1H-pyrrolo[2,3-b]pyridin-4-yl)-1H-indazol-3-amine;
3-(3-amino-5-(1H-pyrrolo[2,3-b]pyridin-4-yl)-1H-indazol-7-yl)prop-2-yn-1-ol;
4-(3-amino-5-(1H-pyrrolo[2,3-b]pyridin-4-yl)-1H-indazol-7-yl)-2-methylbut-3-yn-2-ol;
1-((3-amino-5-(1H-pyrrolo[2,3-b]pyridin-4-yl)-1H-indazol-7-yl)ethynyl)cyclopentan-1-ol;
1-((3-amino-5-(1H-pyrrolo[2,3-b]pyridin-4-yl)-1H-indazol-7-yl)ethynyl)cyclohexan-1-ol;
1-((3-amino-5-(1H-pyrrolo[2,3-b]pyridin-4-yl)-1H-indazol-7-yl)ethynyl)cycloheptan-1-ol;
7-(5-morpholinopent-1-yn-1-yl)-5-(1H-pyrrolo[2,3-b]pyridin-4-yl)-1H-indazol-3-amine;
7-(4-(piperidin-1-yl)but-1-yn-1-yl)-5-(1H-pyrrolo[2,3-b]pyridin-4-yl)-1H-indazol-3-amine;
7-(5-(piperidin-1-yl)pent-1-yn-1-yl)-5-(1H-pyrrolo[2,3-b]pyridin-4-yl)-1H-indazol-3-amine;
7-(6-(piperidin-1-yl)hex-1-yn-1-yl)-5-(1H-pyrrolo[2,3-b]pyridin-4-yl)-1H-indazol-3-amine;
6-(3-amino-5-(1H-pyrrolo[2,3-b]pyridin-4-yl)-1H-indazol-7-yl)hex-5-ynoic acid;
7-(3-amino-5-(1H-pyrrolo[2,3-b]pyridin-4-yl)-1H-indazol-7-yl)hept-6-ynoic acid;
7-(4-phenoxybut-1-yn-1-yl)-5-(1H-pyrrolo[2,3-b]pyridin-4-yl)-1H-indazol-3-amine;
7-(6-phenoxyhex-1-yn-1-yl)-5-(1H-pyrrolo[2,3-b]pyridin-4-yl)-1H-indazol-3-amine;
5-(2-methyl-1H-pyrrolo[2,3-b]pyridin-4-yl)-1H-indazol-3-amine;
5-(2-(tert-butyl)-1H-pyrrolo[2,3-b]pyridin-4-yl)-1H-indazol-3-amine;
5-(2-cyclopropyl-1H-pyrrolo[2,3-b]pyridin-4-yl)-1H-indazol-3-amine;
5-(2-cyclohexyl-1H-pyrrolo[2,3-b]pyridin-4-yl)-1H-indazol-3-amine;
5-(2-neopentyl-1H-pyrrolo[2,3-b]pyridin-4-yl)-1H-indazol-3-amine;
5-(2-(cyclohexylmethyl)-1H-pyrrolo[2,3-b]pyridin-4-yl)-1H-indazol-3-amine;
5-(2-(2-cyclohexylethyl)-1H-pyrrolo[2,3-b]pyridin-4-yl)-1H-indazol-3-amine;
5-(2-benzyl-1H-pyrrolo[2,3-b]pyridin-4-yl)-1H-indazol-3-amine;
(4-(3-amino-1H-indazol-5-yl)-1H-pyrrolo[2,3-b]pyridin-2-yl)methanol;
2-(4-(3-amino-1H-indazol-5-yl)-1H-pyrrolo[2,3-b]pyridin-2-yl)propan-2-ol;
3-(4-(3-amino-1H-indazol-5-yl)-1H-pyrrolo[2,3-b]pyridin-2-yl)pentan-3-ol;
5-(2-(tert-butoxymethyl)-1H-pyrrolo[2,3-b]pyridin-4-yl)-1H-indazol-3-amine;
5-(2-(tetrahydro-2H-pyran-4-yl)-1H-pyrrolo[2,3-b]pyridin-4-yl)-1H-indazol-3-amine;
5-(2-(tetrahydro-2H-pyran-2-yl)-1H-pyrrolo[2,3-b]pyridin-4-yl)-1H-indazol-3-amine;
5-(2-((tetrahydro-2H-pyran-4-yl)methyl)-1H-pyrrolo[2,3-b]pyridin-4-yl)-1H-indazol-3-amine;
4-(3-amino-1H-indazol-5-yl)-1H-pyrrolo[2,3-b]pyridine-2-carboxylic acid;
methyl 4-(3-amino-1H-indazol-5-yl)-1H-pyrrolo[2,3-b]pyridine-2-carboxylate;
ethyl 4-(3-amino-1H-indazol-5-yl)-1H-pyrrolo[2,3-b]pyridine-2-carboxylate;
4-(3-amino-1H-indazol-5-yl)-1H-pyrrolo[2,3-b]pyridine-2-carboxamide;
(4-(3-amino-1H-indazol-5-yl)-1H-pyrrolo[2,3-b]pyridin-2-yl)(pyrrolidin-1-yl)methanone;
4-(3-amino-1H-indazol-5-yl)-N-cyclopentyl-1H-pyrrolo[2,3-b]pyridine-2-carboxamide;
4-(3-amino-1H-indazol-5-yl)-N-cyclohexyl-1H-pyrrolo[2,3-b]pyridine-2-carboxamide;
4-(3-amino-1H-indazol-5-yl)-N-isopentyl-1H-pyrrolo[2,3-b]pyridine-2-carboxamide;
4-(3-amino-1H-indazol-5-yl)-N-phenethyl-1H-pyrrolo[2,3-b]pyridine-2-carboxamide;
4-(3-amino-1H-indazol-5-yl)-N-(3-phenylpropyl)-1H-pyrrolo[2,3-b]pyridine-2-carboxamide;
4-(3-amino-1H-indazol-5-yl)-N-(2-methoxyethyl)-1H-pyrrolo[2,3-b]pyridine-2-carboxamide;
4-(3-amino-1H-indazol-5-yl)-N-(2-aminoethyl)-1H-pyrrolo[2,3-b]pyridine-2-carboxamide;
4-(3-amino-1H-indazol-5-yl)-N-(2-(dimethylamino)ethyl)-1H-pyrrolo[2,3-b]pyridine-2-carboxamide;
(4-(3-amino-1H-indazol-5-yl)-1H-pyrrolo[2,3-b]pyridin-2-yl)(4-methylpiperazin-1-yl)methanone;
4-(3-amino-1H-indazol-5-yl)-N-(2-(piperidin-1-yl)ethyl)-1H-pyrrolo[2,3-b]pyridine-2-carboxamide
4-(3-amino-1H-indazol-5-yl)-N-(2-(butyl(ethyl)amino)ethyl)-1H-pyrrolo[2,3-b]pyridine-2-carboxamide;
4-(3-amino-1H-indazol-5-yl)-N-(2-(diisopropylamino)ethyl)-1H-pyrrolo[2,3-b]pyridine-2-carboxamide;
4-(3-amino-1H-indazol-5-yl)-N-(3-(dimethylamino)propyl)-1H-pyrrolo[2,3-b]pyridine-2-carboxamide;
5-(2-((tert-butylamino)methyl)-1H-pyrrolo[2,3-b]pyridine-4-yl)-1H-indazol-3-amine
5-(2-((isopentylamino)methyl)-1H-pyrrolo[2,3-b]pyridine-4-yl)-1H-indazol-3-amine;
5-(2-(piperidin-2-yl)-1H-pyrrolo[2,3-b]pyridine-4-yl)-1H-indazol-3-amine;
5-(2-((cyclohexylamino)methyl)-1H-pyrrolo[2,3-b]pyridine-4-yl)-1H-indazol-3-amine;
5-(2-((phenylamino)methyl)-1H-pyrrolo[2,3-b]pyridine-4-yl)-1H-indazol-3-amine;
5-(2-(((2-(benzyloxy)phenyl)amino)methyl)-1H-pyrrolo[2,3-b]pyridine-4-yl)-1H-indazol-3-amine;
5-(2-(((2-methoxyethyl)amino)methyl)-1H-pyrrolo[2,3-b]pyridine-4-yl)-1H-indazol-3-amine;
N¹-((4-(3-amino-1H-indazol-5-yl)-1H-pyrrolo[2,3-b]pyridin-2-yl)methyl)-N²,N²-dimethylethane-1,2-diamine;
5-(2-(((3-methoxypropyl)amino)methyl)-1H-pyrrolo[2,3-b]pyridine-4-yl)-1H-indazol-3-amine;
5-(2-(((3-isopropoxypropyl)amino)methyl)-1H-pyrrolo[2,3-b]pyridine-4-yl)-1H-indazol-3-amine;
N¹-((4-(3-amino-1H-indazol-5-yl)-1H-pyrrolo[2,3-b]pyridin-2-yl)methyl)-N³,N³-dimethylpropane-1,3-diamine;
5-(2-((isopropyl(methyl)amino)methyl)-1H-pyrrolo[2,3-b]pyridine-4-yl)-1H-indazol-3-amine;
5-(2-(piperidin-1-ylmethyl)-1H-pyrrolo[2,3-b]pyridine-4-yl)-1H-indazol-3-amine;
5-(2-((4,4-difluoropiperidin-1-yl)methyl)-1H-pyrrolo[2,3-b]pyridine-4-yl)-1H-indazol-3-amine;
5-(2-(morpholinomethyl)-1H-pyrrolo[2,3-b]pyridine-4-yl)-1H-indazol-3-amine;
5-(2-((4-methylpiperazin-1-yl)methyl)-1H-pyrrolo[2,3-b]pyridine-4-yl)-1H-indazol-3-amine;
5-(2-((4-(tert-butyl)piperazin-1-yl)methyl)-1H-pyrrolo[2,3-b]pyridin-4-yl)-1H-indazol-3-amine;
5-(2-(azepan-1-ylmethyl)-1H-pyrrolo[2,3-b]pyridine-4-yl)-1H-indazol-3-amine;
5-(2-((4-methyl-1,4-diazepan-1-yl)methyl)-1H-pyrrolo[2,3-b]pyridin-4-yl)-1H-indazol-3-amine;
5-(2-(2-(piperidin-1-yl)ethyl)-1H-pyrrolo[2,3-b]pyridine-4-yl)-1H-indazol-3-amine;
5-(2-(2-morpholinoethyl)-1H-pyrrolo[2,3-b]pyridin-4-yl)-1H-indazol-3-amine;
5-(2-(3-(piperidin-1-yl)propyl)-1H-pyrrolo[2,3-b]pyridin-4-yl)-1H-indazol-3-amine;
5-(2-(3-(cyclohexylamino)propyl)-1H-pyrrolo[2,3-b]pyridin-4-yl)-1H-indazol-3-amine;
5-(2-(3-morpholinopropyl)-1H-pyrrolo[2,3-b]pyridin-4-yl)-1H-indazol-3-amine;
5-(2-(piperidin-4-ylmethyl)-1H-pyrrolo[2,3-b]pyridin-4-yl)-1H-indazol-3-amine;
5-(2-((1-benzylpiperidin-4-yl)methyl)-1H-pyrrolo[2,3-b]pyridin-4-yl)-1H-indazol-3-amine;
N-(4-(3-Amino-7-(3,3-dimethylbut-1-yn-1-yl)-1H-indazol-5-yl)pyridin-2-yl)oxazol-2-amine;
7-(3,3-Dimethylbut-1-yn-1-yl)-5-(2-((3,3,3-trifluoropropyl)amino)pyridin-4-yl)-1H-indazol-3-amine;
7-(Cyclopropylethynyl)-5-(2-(oxetan-3-ylamino)pyridin-4-yl)-1H-indazol-3-amine;
Methyl (4-(3-amino-7-((tetrahydro-2H-pyran-4-yl)ethynyl)-1H-indazol-5-yl)pyridin-2-yl)carbamate;
Methyl (4-(3-amino-7-((3-methyloxetan-3-yl)ethynyl)-1H-indazol-5-yl)pyridin-2-yl)carbamate;
4-(3-Amino-5-(2-(oxetan-3-ylamino)pyridin-4-yl)-1H-indazol-7-yl)-2-methylbut-3-yn-2-ol;
5-(2-(Oxetan-3-ylamino)pyridin-4-yl)-7-((tetrahydro-2H-pyran-4-yl)ethynyl)-1H-indazol-3-amine;
N-(4-(3-Amino-7-(cyclopropylethynyl)-1H-indazol-5-yl)pyridin-2-yl)cyclopropanecarboxamide;
N-(4-(3-Amino-7-(3-hydroxy-3-methylbut-1-yn-1-yl)-1H-indazol-5-yl)pyridin-2-yl)cyclopropanecarboxamide;
Methyl (4-(3-amino-7-(5-morpholinopent-1-yn-1-yl)-1H-indazol-5-yl)pyridin-2-yl)carbamate;
N-(4-(3-Amino-7-(3-hydroxy-3-methylbutyl)-1H-indazol-5-yl)pyridin-2-yl)cyclopropanecarboxamide;
Methyl (4-(3-amino-7-(3,3-dimethylbutyl)-1H-indazol-5-yl)pyridin-2-yl)carbamate;
5-(2-Cyclopropyl-1H-pyrrolo[2,3-b]pyridin-4-yl)-7-(3,3-dimethylbut-1-yn-1-yl)-1H-indazol-3-amine;
5-(2-Cyclopentyl-1H-pyrrolo[2,3-b]pyridin-4-yl)-7-(3,3-dimethylbut-1-yn-1-yl)-1H-indazol-3-amine;
5-(2-(tert-Butyl)-1H-pyrrolo[2,3-b]pyridin-4-yl)-7-(3,3-dimethylbut-1-yn-1-yl)-1H-indazol-3-amine;
(4-(3-Amino-7-(3,3-dimethylbut-1-yn-1-yl)-1H-indazol-5-yl)-1H-pyrrolo[2,3-b]pyridin-2-yl)methanol;
2-(4-(3-Amino-7-(3,3-dimethylbut-1-yn-1-yl)-1H-indazol-5-yl)-1H-pyrrolo[2,3-b]pyridin-2-yl)propan-2-ol;
Methyl 4-(3-amino-7-(3,3-dimethylbut-1-yn-1-yl)-1H-indazol-5-yl)-1H-pyrrolo[2,3-b]pyridine-2-carboxylate;
5-(2-(Difluoromethyl)-3H-imidazo[4,5-b]pyridin-7-yl)-7-(3,3-dimethylbut-1-yn-1-yl)-1H-indazol-3-amine;
5-(2-Cyclobutyl-3H-imidazo[4,5-b]pyridin-7-yl)-7-(3,3-dimethylbut-1-yn-1-yl)-1H-indazol-3-amine;
N-(4-(3-Amino-7-(3,3-dimethylbut-1-yn-1-yl)-1H-indazol-5-yl)pyridin-2-yl)cyanamide;
5-(2-((1H-Pyrazol-3-yl)amino)pyridin-4-yl)-7-(3,3-dimethylbut-1-yn-1-yl)-1H-indazol-3-amine;
5-(2-((1H-Pyrazol-4-yl)amino)pyridin-4-yl)-7-(3,3-dimethylbut-1-yn-1-yl)-1H-indazol-3-amine;
7-(3,3-Dimethylbut-1-yn-1-yl)-5-(2-((5-methyl-1H-pyrazol-3-yl)amino)pyridin-4-yl)-1H-indazol-3-amine;
7-(3,3-Dimethylbut-1-yn-1-yl)-5-(2-((3-methyl-1H-pyrazol-4-yl)amino)pyridin-4-yl)-1H-indazol-3-amine;
N-(4-(3-Amino-7-(3,3-dimethylbut-1-yn-1-yl)-1H-indazol-5-yl)pyridin-2-yl)thiazol-2-amine;
N-(4-(3-Amino-7-(3,3-dimethylbut-1-yn-1-yl)-1H-indazol-5-yl)pyridin-2-yl)-4-methyloxazol-2-amine;
N-(4-(3-Amino-7-(3,3-dimethylbut-1-yn-1-yl)-1H-indazol-5-yl)pyridin-2-yl)-4-(trifluoromethyl)oxazol-2-amine;
N-(4-(3-amino-7-(3,3-dimethylbut-1-yn-1-yl)-1H-indazol-5-yl)pyridin-2-yl)-3,5-dimethylisoxazol-4-amine;
5-(2-((1H-Imidazol-4-yl)amino)pyridin-4-yl)-7-(3,3-dimethylbut-1-yn-1-yl)-1H-indazol-3-amine;
5-(2-((4H-1,2,4-Triazol-3-yl)amino)pyridin-4-yl)-7-(3,3-dimethylbut-1-yn-1-yl)-1H-indazol-3-amine;
7-(3,3-Dimethylbut-1-yn-1-yl)-5-(2-((2-methyl-2H-tetrazol-5-yl)amino)pyridin-4-yl)-1H-indazol-3-amine;
7-(3,3-Dimethylbut-1-yn-1-yl)-5-(2-(pyrimidin-2-ylamino)pyridin-4-yl)-1H-indazol-3-amine;
7-(3,3-Dimethylbut-1-yn-1-yl)-5-(2-((tetrahydrofuran-3-yl)amino)pyridin-4-yl)-1H-indazol-3-amine;
Methyl (4-(3-Amino-7-(4-hydroxyphenyl)-1H-indazol-5-yl)pyridin-2-yl)carbamate;
4-(3-Amino-5-(2-(oxetan-3-ylamino)pyridin-4-yl)-1H-indazol-7-yl)phenol;
Methyl (4-(3-amino-7-(4-aminophenyl)-1H-indazol-5-yl)pyridin-2-yl)carbamate;
Methyl (4-(7-(4-acetamidophenyl)-3-amino-1H-indazol-5-yl)pyridin-2-yl)carbamate;
Methyl (4-(3-amino-7-(4-carbamoylphenyl)-1H-indazol-5-yl)pyridin-2-yl)carbamate;
Methyl (4-(3-amino-7-(4-(morpholinomethyl)phenyl)-1H-indazol-5-yl)pyridin-2-yl)carbamate;
5-(2-Aminopyridin-4-yl)-7-(4-(2-morpholinoethyl)phenyl)-1H-indazol-3-amine;
Methyl (4-(3-amino-7-(4-(2-morpholinoethyl)phenyl)-1H-indazol-5-yl)pyridin-2-yl)carbamate;
Methyl (4-(3-amino-7-(4-(methylsulfonyl)phenyl)-1H-indazol-5-yl)pyridin-2-yl)carbamate;
Methyl (4-(3-amino-7-(3-hydroxyphenyl)-1H-indazol-5-yl)pyridin-2-yl)carbamate;
Methyl (4-(3-amino-7-(3-carbamoylphenyl)-1H-indazol-5-yl)pyridin-2-yl)carbamate;
Methyl (4-(3-amino-7-(3-(morpholinomethyl)phenyl)-1H-indazol-5-yl)pyridin-2-yl)carbamate;
N-(4-(3-Amino-1H-indazol-5-yl)pyridin-2-yl)-2-cyclohexylacetamide; or
6-Amino-4-(3-amino-7-(3,3-dimethylbut-1-yn-1-yl)-1H-indazol-5-yl)nicotinonitrile.

The various functional groups and substituents making up the compounds of the Formula (I), or sub-formulae (Ia) to (Ie), are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers and mixtures thereof that possess activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and 0 may be in any isotopic form, including 16O and 18O; and the like.

It is also to be understood that certain compounds of the Formula (I), or sub-formulae (Ia) to (Ie), may exist in solvated as well as unsolvated forms such as, for example, hydrated forms. It is to be understood that the invention encompasses all such solvated forms that possess activity.

It is also to be understood that certain compounds of the Formula (I), or sub-formulae (Ia) to (Ie), may exhibit polymorphism, and that the invention encompasses all such forms that possess activity.

Compounds of the Formula (I), or sub-formulae (Ia) to (Ie), may exist in a number of different tautomeric forms and references to compounds of the Formula (I), or sub-formulae (Ia) to (Ie), include all such forms. For the avoidance of doubt, where a compound can exist in one of several tautomeric forms, and only one is specifically described or shown, all others are nevertheless embraced by Formula (I), or sub-formulae (Ia) to (Ie). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

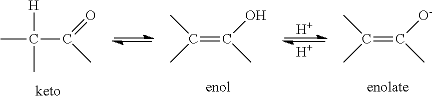

Compounds of the Formula (I), or sub-formulae (Ia) to (Ie), containing an amine function may also form N-oxides. A reference herein to a compound of the Formula (I), or sub-formulae (Ia) to (Ie), that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae (Ia) to (Ie), may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae (Ia) to (Ie), and in-vivo cleavable amide derivatives that may be formed at a carboxy group or an amino group in a compound of the Formula (I), or sub-formulae (Ia) to (Ie).

Accordingly, the present invention includes those compounds of the Formula (I), or sub-formulae (Ia) to (Ie), as defined hereinbefore, when made available by organic synthesis and when made available within the human or animal body by way of cleavage of a pro-drug thereof. Accordingly, the present invention includes those compounds of the Formula (I), or sub-formulae (Ia) to (Ie), that are produced by organic synthetic means and also such compounds that are produced in the human or animal body by way of metabolism of a precursor compound, that is a compound of the Formula (I), or sub-formulae (Ia) to (Ie), may be a synthetically-produced compound or a metabolically-produced compound.

A suitable pharmaceutically acceptable pro-drug of a compound of the Formula (I), or sub-formulae (Ia) to (Ie), is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988); f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae (Ia) to (Ie), that possesses a carboxy group is, for example, an in vivo cleavable ester thereof. An in vivo cleavable ester of a compound of the Formula I, or sub-formulae (Ia) to (Ie), containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae (Ia) to (Ie), that possesses a hydroxy group is, for example, an in vivo cleavable ester or ether thereof. An in vivo cleavable ester or ether of a compound of the Formula (I), or sub-formulae (Ia) to (Ie), containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable pro-drug of a compound of the Formula (I), or sub-formulae (Ia) to (Ie), that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I), or sub-formulae (Ia) to (Ie), that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae (Ia) to (Ie), may be exerted in part by one or more metabolites that are formed within the human or animal body after administration of a compound of the Formula (I), or sub-formulae (Ia) to (Ie). As stated hereinbefore, the in vivo effects of a compound of the Formula (I), or sub-formulae (Ia) to (Ie), may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide.

Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae (Ia) to (Ie), will vary depending on the nature of R¹, R², R³, R⁴, R⁶ and R¹⁰ and any substituent groups or subgroups associated therewith. Suitable processes for their preparation are described further in the accompanying Examples.

Once a compound of Formula (I), or sub-formulae (Ia) to (Ie), has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups may be further reacted to change the nature of the group and provide an alternative compound of Formula (I).

The resultant compounds of Formula (I), or sub-formulae (Ia) to (Ie), can be isolated and purified using techniques well known in the art.

The compounds of Formula (I) may be synthesised by the synthetic routes shown in the Examples section below.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the IKK-alpha in vitro assay described in the Examples section, with preferred compounds showing selectivity for IKK-alpha over IKK-beta.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of IKK activity, particularly IKKα activity. Accordingly, the compounds of the present invention are suitable for the treatment of any disease or condition in which the inhibition of IKKα activity is potentially beneficial.

IKKα activity is known to play a role in cancer.

The role of IKKα in cancer
IKKα in solid tumours

In recent years the role of the non-canonical NF-κB pathway and IKKα within it have increasingly been implicated in the development and progression of multiple solid tumours. The non-canonical NF-κB pathway has been associated with poor prognosis in glioblastoma [57] and mouse orthotopic models have demonstrated that up-regulation of this pathway is associated with an aggressive glioblastoma subtype [57]. In prostate cancer, nuclear localisation of RelB is associated with higher grade tumours [58] and treatment of prostate cancer cells with androgens induces accumulation of nuclear p52 [59]. In addition, silencing of IKKα reduces androgen receptor activity and gene expression, providing evidence that IKKα is associated with prostate cancer growth [58]. Therefore, IKKα is an attractive target for prostate cancer as the androgen receptor is the main driver of prostate cancer proliferation and inhibition of cell death.

In pancreatic cancer, the non-canonical NF-κB pathway is constitutively activated and associated with increased cell proliferation [60]. NIK is elevated in pancreatic cancer and associated with increased proliferation [61, 62] and up-regulation of RelB and p52 are associated with mutated KRAS pancreatic cancer [63] with IKKα-dependent gene expression being observed. In gastrointestinal tumours NF-κB2^{DCT/DCT} mice develop tumours spontaneously, providing evidence that p100/p52 drives oncogenesis in this setting [64]. In renal cancer, members of the non-canonical NF-κB pathway are associated with poor prognosis, increased disease stage and decreased local inflammation [65]. In lung cancer, RelB is associated with shorter overall survival, differentiation, tumour invasion, lymph node metastasis, distant metastasis and ‘tumour, node, metastasis’ (TNM) stage [68]. In bladder cancer, up-regulation of RelB and p52 correlate with histological grade, stage and lymph node metastasis [69].

There are also numerous studies investigating IKKα in breast cancer. IKKα, RelB and p52 are associated with decreased cancer specific survival in ER-positive breast disease [70, 71]. Bcl3 can form a DNA-binding complex with p52 and has been observed as over expressed in breast cancer samples. IKKα is demonstrated to play an essential role in the proliferation of mammary epithelium and it is therefore not surprising that aberrant IKKα signalling has been reported in breast cancer [72]. Yang et al. 2013 reports that in HER2 positive epithelial cells nuclear IKKα can promote progression to tumourogenesis via p27 [73]. In transgenic mice, overexpression of p100/52 results in a delay of mammary gland development, which is accompanied with over expression of cyclin D1, MMP2, MMP9 and COX-2 expression and results in the mice developing multiple tumours [74]. In addition, constitutive RANK signalling causes elevation of non-canonical NF-κB signalling in breast cancer cell lines, which subsequently stimulates cell proliferation via increased transcription of cyclin D1 [75-77] and nuclear IKKα expression is observed in invasive ductal carcinoma and associated with disease free survival. Immuno-histochemical studies have demonstrated that the p52 subunit is expressed at a higher level in the breast cancer tissue compared to normal adjacent tissue [78] and Western blots of nuclear fractions extracted from cancerous and adjacent normal breast tissue confirm an increase in p52 levels in the tumour cells [78]. This is accompanied by an increase in mRNA levels of p52, Bcl-3 and cyclin D1, all genes regulated by IKKα [78]. In addition, IKKα has been demonstrated in cervical, lung, prostate and pancreatic cell lines to regulate mTORC1 and mTORC2 which control tumour cell proliferation [79]. Taken as a whole, there is now a large body of evidence to support the role of the IKKα-NF-κB non-canonical pathway in the development and progression of solid tumours.

### Ikkα Signalling Independent of NF-κB Pathways in Solid Tumours

In addition to the role IKKα plays in NF-κB pathways, it is also reported to have a role independent of both the canonical and non-canonical NF-κB pathways. IKKα accumulates in the nucleus, where it can phosphorylate a variety of substrates including histone H3, SMRT and nuclear co-repressor (NCoR) [80]. In colorectal cancer, IKKα phosphorylates SMRT, resulting in increased expression of Notch dependent genes [80]. In addition, IKKα has been reported to be associated with NOTCH activation in the presence of anti-oestrogens in breast cancer, resulting in up-regulation of ER-dependent gene expression and providing a mechanism for hormone resistance in an NF-κB independent manner [81, 82]. Bennett et al. reported that IKKα expression and not NIK or RelB is associated with recurrence in Luminal A breast cancer, suggesting it is independent of the non-canonical NF-κB pathway [71]. In a second cohort of patients who received tamoxifen, the authors reported that cytoplasmic IKKα was associated with disease-free survival and recurrence-free survival on tamoxifen in Luminal A disease, which may predict patients likely to develop resistance to tamoxifen or IKKα targeted therapies [71] again supporting a role for IKKα in tamoxifen resistant breast cancer. In contrast however, Roseweir et al. reported in the Tamoxifen and Exemestane Adjuvant Multinational (TEAM) clinical trial cohort that low IKKα expression is associated with increased risk of recurrence on sequential tamoxifen/exemestane therapy, suggesting that the role of IKKα in hormone therapy resistance may change depending on the mechanism of action of the therapy the patient receives [83].

In gastric cancer, Helicobacter pylori-mediated NF-κB activation is thought to occur via an IKKα-linked pathway that is independent of the non-canonical NF-κB pathway, but involves both IKKα and NIK to up-regulate inflammatory infiltrate and promote tumourigenesis [84]. Studies of IKKα independent of the non-canonical NF-κB pathway in colorectal cancer and cutaneous squamous cell carcinoma have centred on a truncated form of IKKα (p45 IKKα) that is constitutively active and specifically resides in the nucleus [55, 56]. Bennett et al. observed that nuclear IKKα in breast cancer has a stronger predictive power than cytoplasmic IKKα, and proposed that this could be due to detection of the truncated activated form of p45 IKKα as the antibody employed was unable to distinguish between full length IKKα and the truncated p45 IKKα form [71]. Other studies of IKKα signalling independent of the non-canonical NF-κB pathway in colorectal cancer provide additional evidence that IKKα binds to Notch-dependent gene promoters to upregulate them and release chromatin-bound SMRT, which can be restored by inhibition IKKα and results in colorectal cancer xenografts shrinking in size [56]. It has been reported that the truncated p45 IKKα, forms a complex with full length IKKα and NEMO and is responsible for regulating phosphorylation of SMRT and histone H3 in an NF-κB-independent fashion. In addition, p45 IKKα may be phosphorylated in a TAK1-dependent but NF-κB-independent manner in BRAF^{V600E} mutant colorectal tumours [56], so supporting a role for nuclear IKKα independent of non-canonical NF-κB signalling.

The nuclear role of IKKα is consistently reported as being independent of NF-κB, by activating alternative pathways such as NOTCH [85]. This has been observed in breast cancer, skin cancer and osteosarcoma [86]. In liver cancer Hepatitis B virus X protein down-regulates maspin expression via nuclear IKKα resulting in chemoresistance, suggesting that targeting IKKα could re-sensitise HCC tumours to chemotherapy [87]. In transgenic adenocarcinoma of the mouse prostate (TRAMP) models of prostate cancer IKKα can translocate to the nucleus to promote metastasis and development of castrate resistant disease in a maspin dependent manner, which is accompanied by a local inflammatory response [88]. Similar to breast cancer, in prostate cancer nuclear IKKα appears to provide a mechanism for hormone resistance as IKKα is associated with development of castrate resistant prostate cancer [53] and deletion of BAG3 which is required for IKKα nuclear translocation delays development of castrate resistant disease [89].

Ikkα Association with Hallmarks of Cancer in Human Tumours

The NF-κB pathways regulate the transcription of a wide range of genes involved in the inflammation, proliferation and apoptosis. Many of these processes are hallmarks of cancer [46, 47] and NF-κB has been hypothesised to be a link between inflammation and tumourigenesis. Whether IKKα functions as a member of the non-canonical NF-κB pathway or in its NF-κB-independent roles, it is clear that it is involved with multiple hallmarks of cancer including key roles in innate and adaptive immune responses, cell survival, cell death and inflammation [90, 91]. The non-canonical NF-κB pathway has key roles in regulating processes including production of lymphoid organs (responsible for B and T lymphocyte production), B-cell development and survival, dendritic cell function and bone metabolism [92] and has been reported to promote development and progression of cancers via promotion of inflammatory infiltrate. Mouse model studies have demonstrated that mice with a dominant-negative, catalytically-inactive IKKα, have reduced adenoma formation, smaller colorectal tumours with a lower proliferation index when treated with a carcinogen and this was associated with increased recruitment of macrophages and other immune cell types [93]. In skin cancer studies, IKKα has been demonstrated to induce inflammation-related genes [94]. In an additional study using a model of peritoneal metastasis in immune-competent mice, intraperitoneal injection with IκBa suppressed colon cells induced an M1-like macrophage phenotype, with reduced liver and peritoneal metastases in vivo. This was associated with increased intra-tumoural activated CD4⁺ and CD8⁺ T cells and reduced angiogenesis [93], demonstrating that NF-κB pathways work with local inflammatory infiltrate to promote colorectal cancer progression. In renal cancer the inflammatory effects of the NF-κB pathway have mainly been attributed to the canonical p65/p50 subunits in conjunction with STAT3. However, NIK and RelB have previously been shown to be crucial for B-cell development [2], suggesting that the non-canonical NF-κB pathway also plays a role and that RelB can modulate local inflammatory infiltrate in renal cell carcinoma. IKKα is also associated with promoting expression of pro-inflammatory cytokines such as IL-8 in prostate cancer [95].

Kong et al. suggests that IKKα can be phosphorylated via deleted in breast cancer 1 (DBC1) to regulate B cell activation via RelB activity and causing increased cell proliferation in mice [96]. In addition, polymerase chain reaction (PCR) array-based gene transcriptional profiling experiments demonstrated that reducing cellular IKKα expression had a significant impact on increased expression of genes associated with induction of apoptosis, in particular BAK1 and BBC3, providing evidence that IKKα is involved regulating both cell proliferation and apoptosis in ER positive breast cancer. Dan et al. demonstrates that IKKα via mTORC can induce cell proliferation in cervical, lung, prostate and pancreatic cell lines [79] and in basal cell carcinoma IKKα is associated with proliferation and EMT [94]. Studies in vitro also demonstrate that ovarian cancer epithelial cell proliferation, migration and an invasive phenotype of the cancer were promoted via up-regulation of IKKα. In addition, NIK levels have been associated with regulating both cell proliferation and apoptosis in colorectal cancer, demonstrating that the non-canonical NF-κB pathway is involved in cell viability and tumour growth [97].

### Ikkα in Haematological Malignancies

Aberrant NF-κB signalling and associated gene transcription that modulate cellular processes involved in the initiation, maintenance and progression of human malignancies are also common to haematological cells and cancers. In this regard, many B-cell leukaemias and lymphomas display abnormal NF-κB activation, implicating this family of transcription factors in these diseases and suggesting regulation of these proteins may represent promising therapeutic targets. In addition, it is now appreciated that conventional cytotoxic agents can increase NF-κB activation, contributing to the development of drug resistance via a number of distinct mechanisms. Therefore, inhibitors that target NIK-IKKα-mediated signalling may prove clinically useful as single agents and also to re-sensitise patients to chemotherapeutic drugs. Given the frequency of genetic mutations in the non-canonical NF-κB pathway and its critical role in tumour microenvironmental signalling, IKKα represents an attractive anti-cancer target.

Chronic lymphocytic leukaemia (CLL) is the commonest leukaemia in Europe and North America. It is characterised by the accumulation of mature-looking CD5⁺/CD19⁺ B lymphocytes in the peripheral blood, bone marrow, and lymphoid tissues [105]. NF-κB is constitutively activated in many CLL patients and this is associated with more aggressive disease [106, 107]. A number of recurrent genetic mutations in NF-κB-associated genes have been described in CLL. The most common of these is an inactivating mutation in NFKBIE that encodes IκBε, a negative NF-κB regulator. These NFKBIE aberrations are found in approximately 7% of CLL cases and predominantly occur in poor-prognostic subgroups. This may be causal as mutations in NFKBIE result in increased nuclear translocation of RelA [108]. NOTCH1 mutations occur at an even higher frequency in CLL (−11%). These activating mutations are associated with poor response to chemotherapy [109] and this may be caused by NOTCH1-mediated NF-κB pathway activation [110-112]. BIRC3 mutations are found in a smaller proportion of CLL patients (−4%) but they impact upon the non-canonical NF-κB pathway due to the premature truncation of the BIRC3-encoded protein product, cIAP2, resulting in the loss of its E3 ubiquitin ligase activity that is essential for NIK proteasomal degradation. As a consequence, NIK levels increase leading to the phosphorylation of IKKα, NF-κB2, the processing of p100 to p52 and the constitutive activation of non-canonical NF-κB signalling [113]. Importantly, BIRC3 mutations are associated with loss of sensitivity to chemotherapy and poor prognosis [114].

In addition to the genetic causes of NF-κB dysregulation in CLL, it is now understood that the lymph node microenvironment plays a critical role in modulating the natural pathology of this disease. Signalling via the B-cell receptor (BCR), toll-like receptors (TLR) and CD40, as well as engagement of the BAFF and a proliferation-inducing ligand (APRIL) receptors TACI, BAFF-R and BCMA, create a pro-survival, pro-proliferative niche mediated by NF-κB activation [116, 117]. The importance of this microenvironment is perhaps best exemplified by the remarkable clinical effects of the Bruton's tyrosine kinase inhibitor ibrutinib. Treatment with this drug results in a marked tissue redistribution effect with leukaemia cells being excluded from the lymphoid tissues [118]. The partitioning of the tumour away from the sites of increased NF-κB signalling results in durable remissions, an effect that is reversed on drug withdrawal.

Diffuse large B-cell lymphomas (DLBCL) are the most common types of non-Hodgkin lymphoma. They are divided into three molecular sub-types: ABC (activated B-cell), GCB (germinal centre B-cell) and PMBL (primary mediastinal B-cell lymphoma). Initial evidence for the role of the canonical NF-κB pathway in DLBCL came from gene expression profiling studies, which showed enrichment for NF-κB target genes in the ABC sub-type. This group has the worst prognosis implicating NF-κB as a modulator of clinical outcome in DLBCL [123]. Constitutive NF-κB activation in the ABC sub-type can result from mutations in components of the BCR signalling cascade, which results in chronic BCR activation. These mutations often occur in the immunoreceptor tyrosine-based motif (ITAM) but also in the coiled-coil domain of the CARD11/CARMA1 gene [124]. Finally, MYD88 gene mutations are found in approximately 30% of the ABC sub-type resulting in spontaneous activation of the downstream IRAK complex and NF-κB activation [125]. The non-canonical NF-κB pathway is also aberrantly dysregulated in 10-15% of DLBCL cases due to TRAF2 and TRAF3 mutations [126] and consequently identifies a sub-population of tumours that may be targetable via IKKα.

Multiple myeloma (MM) is an incurable plasma cell malignancy accounting for approximately 13% of all haematological cancers. Disease progression involves clonal expansion of transformed plasma cells in the bone marrow. Overall, genetic abnormalities leading to constitutive NF-κB activity have been found in approximately 20% of MM patients and 40% of MM cell lines [127-129]. Most of the genetic abnormalities relating to NF-κB dysregulation in MM involve the non-canonical NF-κB pathway including aberrant expression of NIK, CD40, TRAF2, TRAF3, transmembrane activator and CAML interactor (TACI) and cIAP1/2 [127, 128]. In these studies, the majority of MM cases possessed overexpression of the positive NF-κB regulators NIK, TACI and CD40, or reduced or silenced activity of the negative NF-κB regulators TRAF2, TRAF3 and clAP1/2. All of these phenotypes contribute to increased NF-κB signalling, with a preference towards non-canonical NF-κB signalling [128, 129]. In addition, other less common genetic abnormalities that also lead to constitutive NF-κB signalling in MM have been identified. These included high expression of the NFKB1 gene (p105) and abnormalities within the NFKB2 gene (p100), which results in increased canonical and non-canonical NF-κB signalling, respectively [127-129].

Although genetic abnormalities can explain some of the high NF-κB activity in MM, it is likely that a substantial portion of the NF-κB signalling in this disease arises as a consequence of interactions within the bone marrow microenvironment [129]. One such mechanism for NF-κB activation is via CD40-CD40L interactions [130, 131]. CD40 is a cell surface marker not usually expressed on normal plasma cells but has been shown to be increased in the early stages of MM [132]. Furthermore, blocking the interaction of CD40 with CD40L decreases NF-κB activation [127]. This results in the inhibition of IL-6 and vascular endothelial growth factor (VEGF) secretion, which in turn leads to growth arrest and cell death of MM cells [133].

Furthermore, the bone marrow stromal cells (BMSC) found in the MM tumour microenvironment have also been found to express high levels of NF-κB activation that helps to support the proliferation, survival and drug resistance of malignant plasma cells within the bone marrow niche [134]. Adherence of MM cells to BMSCs induces NF-κB-dependent cytokine transcription and secretion of TNFa, IL-6, VEGF, RANKL and BAFF, to promote MM cell survival and growth through MM cell NF-κB activation [135, 136].

### REFERENCES

[57] Duran C L, Lee D W, Jung J U, Ravi S, Pogue C B, Toussaint L G, Bayless K J, Sitcheran R. NIK regulates MT1-MMP activity and promotes glioma cell invasion independently of the canonical NF-κB pathway. Oncogenesis. 2016 Jun. 6; 5(6):e231.
[58] Cherry E, Lee D, Jung J, Sitcheran R. Non-canonical nf-kb signaling drives the aggressive invasiveness of glioblastoma Neuro-oncology 2014 16 (Suppl 5), v2.
[59] Lessard L, Bégin L R, Gleave M E, Mes-Masson A M, Saad F. Nuclear localisation of nuclear factor-kappaB transcription factors in prostate cancer: an immunohistochemical study. Br J Cancer. 2005 Oct. 31; 93(9):1019-23.
[60] Lessard L, Saad F, Le Page C, Diallo J S, Péant B, Delvoye N, Mes-Masson A M. NF-kappaB2 processing and p52 nuclear accumulation after androgenic stimulation of LNCaP prostate cancer cells. Cell Signal. 2007 May; 19(5):1093-100.
[61]Döppler H, Liou G Y, Storz P. Downregulation of TRAF2 mediates NIK-induced pancreatic cancer cell proliferation and tumorigenicity. PLoS One. 2013; 8(1):e53676.
[61] Thu Y M, Richmond A. NF-κB inducing kinase: a key regulator in the immune system and in cancer. Cytokine Growth Factor Rev. 2010 August; 21(4):213-26.
[62] Nishina T, Yamaguchi N, Gohda J, Semba K, Inoue J. NIK is involved in constitutive activation of the alternative NF-kappaB pathway and proliferation of pancreatic cancer cells. Biochem Biophys Res Commun. 2009 Oct. 9; 388(1):96-101.
[63] Chandler N M, Canete J J, Callery M P. Increased expression of NF-kappa B subunits in human pancreatic cancer cells. J Surg Res. 2004 May 1; 118(1):9-14
[64] Ishikawa H, Akedo I, Suzuki T, Narahara H, Otani T. Adverse effects of sulindac used for prevention of colorectal cancer. J Natl Cancer Inst. 1997 Sep. 17; 89(18):1381.
[65] Lua J, Qayyum, T., Edwards, J. and Roseweir, A. K. The prognostic role of the non-canonical NF-kappa B pathway in renal cell carcinoma patients. Urologia Internationalis. 2018: accepted.
[66] Jamieson S, Fuller P J. Characterization of the inhibitor of kappaB kinase (IKK) complex in granulosa cell tumors of the ovary and granulosa cell tumor-derived cell lines. Horm Cancer. 2013 October: 4:277-92.
[67] Cildir G, Low K C, Tergaonkar V. Noncanonical NF-kappa B Signaling in Health and Disease. Trends Mol Med. 2016 May: 22:414-29.
[68] Qin H, Zhou J, Zhou P, Xu J, Tang Z, Ma H, Guo F. Prognostic significance of RelB overexpression in non-small cell lung cancer patients. Thorac Cancer. 2016 July; 7(4):415-21
[69] Shen M, Duan X, Zhou P, Zhou W, Wu X, Xu S, Chen Y, Tao Z. Lymphotoxin p receptor activation promotes bladder cancer in a nuclear factor-KB-dependent manner. Mol Med Rep. 2015 February; 11(2):783-90.
[70] Sovak M A, Bellas R E, Kim D W, Zanieski G J, Rogers A E, Traish A M, Sonenshein G E. Aberrant nuclear factor-kappaB/Rel expression and the pathogenesis of breast cancer. J Clin Invest. 1997 Dec. 15; 100(12):2952-60.
[71] Bennett L, Quinn J, McCall P, Mallon E A, Horgan P G, McMillan D C, Paul A, Edwards J. High IKKα expression is associated with reduced time to recurrence and cancer specific survival in oestrogen receptor (ER)-positive breast cancer. Int J Cancer. 2017 Apr. 1; 140(7):1633-1644.
[72] Cao Y, Bonizzi G, Seagroves T N, Greten F R, Johnson R, Schmidt E V, Karin M. IKKalpha provides an essential link between RANK signaling and cyclin D1 expression during mammary gland development. Cell. 2001 Dec. 14; 107(6):763-75.
[73] Yang Z, Wang X L, Bai R, Liu W Y, Li X, Liu M, Tang H. miR-23a promotes IKKα expression but suppresses ST7L expression to contribute to the malignancy of epithelial ovarian cancer cells. Br J Cancer. 2016 Sep. 6; 115(6):731-40.
[74] Connelly L, Robinson-Benion C, Chont M, Saint-Jean L, Li H, Polosukhin V V, Blackwell T S, Yull F E. A transgenic model reveals important roles for the NF-kappa B alternative pathway (p100/p52) in mammary development and links to tumorigenesis. J Biol Chem. 2007 Mar. 30; 282(13):10028-35.
[75] Karin M, Bonnizi G, Cao Y. NF-kB: a factor that provides a link between stress, inflammation and cancer. European Journal of Cancer. 2002 November: 38:S116.
[76] Karin M, Cao Y X, Greten F R, Li Z W. NF-kappa B in cancer: From innocent bystander to major culprit. Nature Reviews Cancer. 2002 April: 2:301-10.
[77] Karin M, Lin A. NF-kappa B at the crossroads of life and death. Nat Immunol. 2002 March: 3:221-7.
[78] Cogswell P C, Guttridge D C, Funkhouser W K, Baldwin A S, Jr. Selective activation of NF-kappa B subunits in human breast cancer: potential roles for NF-kappa B2/p52 and for Bcl-3. Oncogene. 2000 February 24: 19:1123-31.
[79] Dan H C, Antonia R J, Baldwin A S. PI3K/Akt promotes feedforward mTORC2 activation through IKK alpha. Oncotarget. 2016 April 19: 7:21064-75.
[80] Espinosa L, Margalef P, Bigas A. Non-conventional functions for NF-kappa B members: the dark side of NF-kappa B. Oncogene. 2015 April 30: 34:2279-87.
[81] Rizzo P, Miao H, D'Souza G, Osipo C, Song L L, Yun J, Zhao H, Mascarenhas J, Wyatt D, Antico G, Hao L, Yao K, Rajan P, Hicks C, Siziopikou K, Selvaggi S, Bashir A, Bhandari D, Marchese A, Lendahl U, Qin J Z, Tonetti D A, Albain K, Nickoloff B J, Miele L. Cross-talk between notch and the estrogen receptor in breast cancer suggests novel therapeutic approaches. Cancer Res. 2008 Jul. 1; 68(13):5226-35.
[82] Hao L, Rizzo P, Osipo C, Pannuti A, Wyatt D, Cheung L W, Sonenshein G, Osborne B A, Miele L. Notch-1 activates estrogen receptor-alpha-dependent transcription via IKKalpha in breast cancer cells. Oncogene. 2010 Jan. 14; 29(2):201-13.
[83] Roseweir A K, Bennett L, Dickson A, Cheng K, Quintayo M A, Bayani J, McMillan D C, Horgan P G, van de Velde C J H, Seynaeve C, Hasenburg A, Kieback D G, Markopoulos C, Dirix L Y, Rea D W, Mallon E A, Bartlett J M S, Edwards J. Predictive Biomarkers for Endocrine Therapy: Retrospective Study in Tamoxifen and Exemestane Adjuvant Multinational (TEAM) Trial. J Natl Cancer Inst. 2018 Jun. 1; 110(6):616-627.
[84] Merga Y J, O'Hara A, Burkitt M D, Duckworth C A, Probert C S, Campbell B J, Pritchard D M. Importance of the alternative NF-κB activation pathway in inflammation-associated gastrointestinal carcinogenesis. Am J Physiol Gastrointest Liver Physiol. 2016 Jun. 1; 310(11):G1081-90.
[85] STRAP Promotes Stemness of Human Colorectal Cancer via Epigenetic Regulation of the NOTCH Pathway. Jin L, Vu T, Yuan G, Datta P K. Cancer Res. 2017 Oct. 15; 77(20):5464-5478.
[86] Leopizzi M, Cocchiola R, Milanetti E, Raimondo D, Politi L, Giordano C, Scandurra R, Scotto d'Abusco A. IKKα inibition by a glucosamine derivative enhances Maspin expression in osteosarcoma cell line. Chem Biol Interact. 2017 Jan. 25; 262:19-28.
[87] Cheng K K W, Bennett L, Edwards J. Identification of a novel biomarker of IKK alpha-dependent NF-kappa B signalling in oestrogen receptor (ER)-positive breast cancer. Scot Med J. 2016 November: 61:Np55.
[88] Luo J L, Tan W, Ricono J M, Korchynskyi O, Zhang M, Gonias S L, Cheresh D A, Karin M. Nuclear cytokine-activated IKKalpha controls prostate cancer metastasis by repressing Maspin. Nature. 2007 Apr. 5; 446(7136):690-4.
[89] Ammirante M, De Laurenzi V, Graziano V, Turco M C, Rosati A. BAG3 is required for IKKα nuclear translocation and emergence of castration resistant prostate cancer. Cell Death Dis. 2011 Mar. 31; 2:e139.
[90] Rizel L, Safieh C, Shalev S A, Mezer E, Jabaly-Habib H, Ben-Neriah Z, Chervinsky E, Briscoe D, Ben-Yosef T. Novel mutations of MYO7A and USH1G in Israeli Arab families with Usher syndrome type 1. Mol Vis. 2011; 17:3548-55.
[91] Ben-Neriah Y, Karin M. Inflammation meets cancer, with NF-kappa B as the matchmaker. Nat Immunol. 2011 August: 12:715-23.
[92] Karin M, Greten F R. NF kappa B: Linking inflammation and immunity to cancer development and progression. Nature Reviews Immunology. 2005 October: 5:749-59
[93] Sepulveda A, Soriano H, Espino A. Gastrointestinal tract involvement in Klippel-Trenaunay syndrome. Lancet Gastroenterol Hepatol. 2018 July; 3(7):518.
[94] Jia J, Shi Y, Yan B, Xiao D, Lai W, Pan Y, Jiang Y, Chen L, Mao C, Zhou J, Xi S, Cao Y, Liu S, Tao Y. LGR5 expression is controlled by IKKα in basal cell carcinoma through activating STAT3 signaling pathway. Oncotarget. 2016 May 10; 7(19):27280-94.
[95] Manna S, Singha B, Phyo S A, Gatla H R, Chang T P, Sanacora S, Ramaswami S, Vancurova I. Proteasome inhibition by bortezomib increases IL-8 expression in androgen-independent prostate cancer cells: the role of IKKα. J Immunol. 2013 Sep. 1; 191(5):2837-46.
[96] Kong S, Dong H, Song J, Thiruppathi M, Prabhakar B S, Qiu Q, Lin Z, Chini E, Zhang B, Fang D. Deleted in Breast Cancer 1 Suppresses B Cell Activation through RelB and Is Regulated by IKKα Phosphorylation. J Immunol. 2015 Oct. 15; 195(8):3685-93.
[97] Qu L L, He L, Zhao X, Xu W. Downregulation of miR-518a-3p activates the NIK-dependent NF-kappa B pathway in colorectal cancer. Int J Mol Med. 2015 May: 35:1266-72.
[98] Frelin C, Imbert V, Griessinger E, Peyron A C, Rochet N, Philip P, Dageville C, Sirvent A, Hummelsberger M, Berard E, Dreano M, Sirvent N, Peyron J F. Targeting NF-kappaB activation via pharmacologic inhibition of IKK2-induced apoptosis of human acute myeloid leukemia cells. Blood. 2005 Jan. 15; 105(2):804-11
[99] Hehner S P, Hofmann T G, Dröge W, Schmitz M L. The antiinflammatory sesquiterpene lactone parthenolide inhibits NF-kappa B by targeting the I kappa B kinase complex. J Immunol. 1999 Nov. 15; 163(10):5617-23
[100] Hideshima T, Chauhan D, Kiziltepe T, Ikeda H, Okawa Y, Podar K, Raje N, Protopopov A, Munshi N C, Richardson P G, Carrasco R D, Anderson K C. Biologic sequelae of I{kappa}B kinase (IKK) inhibition in multiple myeloma: therapeutic implications. Blood. 2009 May 21; 113(21):5228-36.
[101] Coope H J, Atkinson P G, Huhse B, Belich M, Janzen J, Holman M J, Klaus G G, Johnston L H, Ley S C. CD40 regulates the processing of NF-kappaB2 p100 to p52. 2002 Oct. 15; 21(20):5375-85.
[102] Kayagaki N, Yan M, Seshasayee D, Wang H, Lee W, French D M, Grewal I S, Cochran A G, Gordon N C, Yin J, Starovasnik M A, Dixit V M. BAFF/BLyS receptor 3 binds the B cell survival factor BAFF ligand through a discrete surface loop and promotes processing of NF-kappaB2. 2002 October; 17(4):515-24
[103] Novack D V, Yin L, Hagen-Stapleton A, Schreiber R D, Goeddel D V, Ross F P, Teitelbaum S L. The IkappaB function of NF-kappaB2 p100 controls stimulated osteoclastogenesis. 2003 Sep. 1; 198(5):771-81
[104] Sun S. C. The non-canonical NF-κB pathway. Immunol. Rev. 2012; 246:125-140.
[105] Scarfò L, Ferreri A J, Ghia P. Chronic lymphocytic leukaemia. Crit Rev Oncol Hematol. 2016; 104:169-82.
[106] Cuni S., Pérez-Aciego P., Pérez-Chacón G., Vargas J. A., Sánchez A., Martin-Saavedra F. M., Ballester S., Garcia-Marco J., Jorde J., Durentez A. A sustained activation of PI3K/NF-κB pathway is critical for the survival of chronic lymphocytic leukemia B cells. Leukemia. 2004; 18:1391-1400.
[107] Hewamana S, Alghazal S, Lin T T, Clement M, Jenkins C, Guzman M L, Jordan C T, Neelakantan S, Crooks P A, Burnett A K, Pratt G, Fegan C, Rowntree C, Brennan P, Pepper C. The NF-kappaB subunit Rel A is associated with in vitro survival and clinical disease progression in chronic lymphocytic leukemia and represents a promising therapeutic target. Blood. 2008 May 1; 111(9):4681-9.
[108] Mansouri L, Sutton L A, Ljungström V, Bondza S, Arngården L, Bhoi S, Larsson J, Cortese D, Kalushkova A, Plevova K, Young E, Gunnarsson R, Falk-Sörqvist E, Lönn P, Muggen A F, Yan X J, Sander B, Enblad G, Smedby K E, Juliusson G, Belessi C, Rung J, Chiorazzi N, Strefford J C, Langerak A W, Pospisilova S, Davi F, Hellström M, Jernberg-Wiklund H, Ghia P, Söderberg O, Stamatopoulos K, Nilsson M, Rosenquist R. Functional loss of IκBε leads to NF-κB deregulation in aggressive chronic lymphocytic leukemia. J Exp Med. 2015 Jun. 1; 212(6):833-43
[109] Fabbri G, Rasi S, Rossi D, Trifonov V, Khiabanian H, Ma J, Grunn A, Fangazio M, Capello D, Monti S, Cresta S, Gargiulo E, Forconi F, Guarini A, Arcaini L, Paulli M, Laurenti L, Larocca L M, Marasca R, Gattei V, Oscier D, Bertoni F, Mullighan C G, Foe R, Pasqualucci L, Rabadan R, Dalla-Favera R, Gaidano G. Analysis of the chronic lymphocytic leukemia coding genome: role of NOTCH1 mutational activation. J Exp Med. 2011 Jul. 4; 208(7):1389-401.
[110] Rosati E, Sabatini R, Rampino G, Tabilio A, Di lanni M, Fettucciari K, Bartoli A, Coaccioli S, Screpanti I, Marconi P. Constitutively activated Notch signaling is involved in survival and apoptosis resistance of B-CLL cells. Blood. 2009 Jan. 22; 113(4):856-65
[111] Baliakas P, Hadzidimitriou A, Sutton L A, Rossi D, Minga E, Villamor N, Larrayoz M, Kminkova J, Agathangelidis A, Davis Z, Tausch E, Stalika E, Kantorova B, Mansouri L, Scarfò L, Cortese D, Navrkalova V, Rose-Zerilli M J, Smedby K E, Juliusson G, Anagnostopoulos A, Makris A M, Navarro A, Delgado J, Oscier D, Belessi C, Stilgenbauer S, Ghia P, Pospisilova S, Gaidano G, Campo E, Strefford J C, Stamatopoulos K, Rosenquist R. Recurrent mutations refine prognosis in chronic lymphocytic leukemia. European Research Initiative on CLL (ERIC). Leukemia. 2015 February; 29(2):329-36
[112] Chiaretti S, Marinelli M, Del Giudice I, Bonina S, Piciocchi A, Messina M, Vignetti M, Rossi D, Di Maio V, Mauro F R, Guarini A, Gaidano G, Foa R. NOTCH1, SF3B1, BIRC3 and TP53 mutations in patients with chronic lymphocytic leukemia undergoing first-line treatment: correlation with biological parameters and response to treatment. Leuk Lymphoma. 2014 December; 55(12):2785-92
[113] Dejardin E. Biochem Pharmacol. The alternative NF-kappaB pathway from biochemistry to biology: pitfalls and promises for future drug development. 2006; 72(9):1161-79.
[114] Rossi D1, Rasi S, Fabbri G, Spina V, Fangazio M, Forconi F, Marasca R, Laurenti L, Bruscaggin A, Cerri M, Monti S, Cresta S, Fama R, De Paoli L, Bulian P, Gattei V, Guarini A, Deaglio S, Capello D, Rabadan R, Pasqualucci L, Dalla-Favera R, Foa R, Gaidano G. Mutations of NOTCH1 are an independent predictor of survival in chronic lymphocytic leukemia. Blood. 2012 Jan. 12; 119(2):521-9.
[115] Puente X S, Pinyol M, Quesada V, Conde L, Ordöñez G R, Villamor N, Escaramis G, Jares P, Beà S, González-Diaz M, Bassaganyas L, Baumann T, Juan M, López-Guerra M, Colomer D, Tubio J M, López C, Navarro A, Tornador C, Aymerich M, Rozman M, Hernendez J M, Puente D A, Freije J M, Velasco G, Gutiérrez-Fernández A, Costa D, Carrió A, Guijarro S, Enjuanes A, Hernández L, YagQe J, Nicolás P, Romeo-Casabona C M, Himmelbauer H, Castillo E, Dohm J C, de Sanjosé S, Piris M A, de Alava E, San Miguel J, Royo R, Gelpi J L, Torrents D, Orozco M, Pisano D G, Valencia A, Guigó R, Bayes M, Heath S, Gut M, Klatt P, Marshall J, Raine K, Stebbings L A, Futreal P A, Stratton M R, Campbell P J, Gut I, López-Guillermo A, Estivill X, Montserrat E, López-Otin C, Campo E. Whole-genome sequencing identifies recurrent mutations in chronic lymphocytic leukaemia. 2011 Jun. 5; 475(7354):101-5.
[116] Herishanu Y, Pérez-Galen P, Liu D, Biancotto A, Pittaluga S, Vire B, Gibellini F, Njuguna N, Lee E, Stennett L, Raghavachari N, Liu P, McCoy J P, Raffeld M, Stetler-Stevenson M, Yuan C, Sherry R, Arthur D C, Maric I, White T, Marti G E, Munson P, Wilson W H, Wiestner A. The lymph node microenvironment promotes B-cell receptor signaling, NF-kappaB activation, and tumor proliferation in chronic lymphocytic leukemia. Blood. 2011 Jan. 13; 117(2):563-74.
[117] Rosén A., Murray F., Evaldsson C., Rosenquist R. Antigens in chronic lymphocytic leukemia-Implications for cell origin and leukemogenesis. Semin. Cancer Biol. 2010; 20:400-409.
[118] Wodarz D, Garg N, Komarova N L, Benjamini O, Keating M J, Wierda W G, Kantarjian H, James D, O'Brien S, Burger J A. Kinetics of CLL cells in tissues and blood during therapy with the BTK inhibitor ibrutinib. Blood. 2014 Jun. 26; 123(26):4132-5.
[119] Lucas P C, Kuffa P, Gu S, Kohrt D, Kim D S, Siu K, Jin X, Swenson J, McAllister-Lucas L M. A dual role for the API2 moiety in API2-MALT1-dependent NF-kappaB activation: heterotypic oligomerization and TRAF2 recruitment. 2007 Aug. 16; 26(38):5643-54.
[120] Rosebeck S, Madden L, Jin X, Gu S, Apel I J, Appert A, Hamoudi R A, Noels H, Sagaert X, Van Loo P, Baens M, Du M Q, Lucas P C, McAllister-Lucas L M. Cleavage of NIK by the API2-MALT1 fusion oncoprotein leads to noncanonical NF-kappaB activation. 2011 January 28; 331(6016):468-72.
[121] Spina V, Rossi D. NF-κB deregulation in splenic marginal zone lymphoma. Semin. Cancer Biol. 2016; 39:61-67.
[122] Thu Y M, Richmond A. NF-κB inducing kinase: a key regulator in the immune system and in cancer. Cytokine Growth Factor Rev. 2010; 21(4):213-26.
[123] Davis R E, Brown K D, Siebenlist U, Staudt L M. Constitutive nuclear factor K B activity is required for survival of activated B cell-like diffuse large B cell lymphoma cells. J. Exp. Med. 2001; 194:1861-1874.
[124] Lenz G, Davis R E, Ngo V N, Lam L, George T C, Wright G W, Dave S S, Zhao H, Xu W, Rosenwald A, Ott G, Muller-Hermelink H K, Gascoyne R D, Connors J M, Rimsza L M, Campo E, Jaffe E S, Delabie J, Smeland E B, Fisher R I, Chan W C, Staudt L M. Oncogenic CARD11 mutations in human diffuse large B cell lymphoma. Science. 2008 Mar. 21; 319(5870):1676-9.
[125] Ngo V N, Young R M, Schmitz R, Jhavar S, Xiao W, Lim K H, Kohlhammer H, Xu W, Yang Y, Zhao H, Shaffer A L, Romesser P, Wright G, Powell J, Rosenwald A, Muller-Hermelink H K, Ott G, Gascoyne R D, Connors J M, Rimsza L M, Campo E, Jaffe E S, Delabie J, Smeland E B, Fisher R I, Braziel R M, Tubbs R R, Cook J R, Weisenburger D D, Chan W C, Staudt L M. Oncogenically active MYD88 mutations in human lymphoma. Nature. 2011 Feb. 3; 470(7332):115-9.
[126] Zhang B, Calado D P, Wang Z, Fröhler S, Köchert K, Qian Y, Koralov S B, Schmidt-Supprian M, Sasaki Y, Unitt C, Rodig S, Chen W, Dalla-Favera R, Alt F W, Pasqualucci L, Rajewsky K. An oncogenic role for alternative NF-κB signaling in DLBCL revealed upon deregulated BCL6 expression. Cell Rep. 2015 May 5; 11(5):715-26.
[127] Annunziata C M, Davis R E, Demchenko Y, Bellamy W, Gabrea A, Zhan F, Lenz G, Hanamura I, Wright G, Xiao W, Dave S, Hurt E M, Tan B, Zhao H, Stephens O, Santra M, Williams D R, Dang L, Barlogie B, Shaughnessy J D Jr, Kuehl W M, Staudt L M. Frequent engagement of the classical and alternative NF-kappaB pathways by diverse genetic abnormalities in multiple myeloma. Cancer Cell. 2007; 12(2):115-30.
[128] Keats J J, Fonseca R, Chesi M, Schop R, Baker A, Chng W J, Van Wier S, Tiedemann R, Shi C X, Sebag M, Braggio E, Henry T, Zhu Y X, Fogle H, Price-Troska T, Ahmann G, Mancini C, Brents L A, Kumar S, Greipp P, Dispenzieri A, Bryant B, Mulligan G, Bruhn L, Barrett M, Valdez R, Trent J, Stewart A K, Carpten J, Bergsagel P L. Promiscuous mutations activate the noncanonical NF-kappaB pathway in multiple myeloma. Cancer Cell. 2007; 12(2):131-44.
[129] Demchenko Y N, Glebov O K, Zingone A, Keats J J, Bergsagel P L, Kuehl W M. Classical and/or alternative NF-kappaB pathway activation in multiple myeloma. Blood. 2010; 115(17):3541-52.
[130] Coope H J1, Atkinson P G, Huhse B, Belich M, Janzen J, Holman M J, Klaus G G, Johnston L H, Ley S C. CD40 regulates the processing of NF-kappaB2 p100 to p52. EMBO J. 2002; 21(20):5375-85.
[131] Hauer J, Püschner S, Ramakrishnan P, Simon U, Bongers M, Federle C, Engelmann H. TNF receptor (TNFR)-associated factor (TRAF) 3 serves as an inhibitor of TRAF2/5-mediated activation of the noncanonical NF-kappaB pathway by TRAF-binding TNFRs. Proc Natl Acad Sci USA. 2005; 102(8):2874-9.
[132]Pérez-Andres M1, Almeida J, Martin-Ayuso M, De Las Heras N, Moro M J, Martin-Nunez G, Galende J, Cuello R, Abuin I, Moreno I, Dominguez M, Hernandez J, Mateo G, San Miguel J F, Orfao A. Soluble and membrane levels of molecules involved in the interaction between clonal plasma cells and the immunological microenvironment in multiple myeloma and their association with the characteristics of the disease. Int J Cancer. 2009; 124(2):367-75.
[133] Richardson P, Schlossman R, Jagannath S, Alsina M, Desikan R, Blood E, Weller E, Mitsiades C, Hideshima T, Davies F, Doss D, Freeman A, Bosch J, Patin J, Knight R, Zeldis J, Dalton W, Anderson K. Thalidomide for patients with relapsed multiple myeloma after high-dose chemotherapy and stem cell transplantation: results of an open-label multicenter phase 2 study of efficacy, toxicity, and biological activity. Mayo Clin Proc. 2004; 79(7):875-82.
[134] McMillin D W, Negri J M, Mitsiades C S. The role of tumour-stromal interactions in modifying drug response: challenges and opportunities. Nat Rev Drug Discov. 2013; 12(3):217-28.
[135] Chauhan D, Uchiyama H, Akbarali Y, Urashima M, Yamamoto K, Libermann T A, Anderson K C. Multiple myeloma cell adhesion-induced interleukin-6 expression in bone marrow stromal cells involves activation of NF-kappa B. Blood. 1996; 87(3):1104-12.
[136] Bommert K, Bargou R C, StQhmer T. Signalling and survival pathways in multiple myeloma. Eur J Cancer. 2006; 42(11):1574-80.

Thus, according to a further aspect of the present invention, there is provided a method of inhibiting IKKα activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which IKKα activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, as defined herein for use in the inhibition of IKKα activity.

According to a further aspect of the present invention, there is provided a compound, or a pharmaceutically acceptable salt, hydrate or solvate thereof, as defined herein for use in the treatment of a disease or disorder in which IKKα activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided a use of a compound, or a pharmaceutically acceptable salt, hydrate or solvate thereof, as defined herein in the manufacture of a medicament for the inhibition of IKKα activity.

According to a further aspect of the present invention, there is provided a use of a compound, or a pharmaceutically acceptable salt, hydrate or solvate thereof, as defined herein in the manufacture of a medicament for the treatment of a disease or disorder in which IKKα activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In particular, the compounds of the present invention will be useful for the treatment of any cancer in which a mis-match repair inhibition is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dube Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor-GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

Particular cancers in which IKKα inhibition is anticipated to be beneficial include advanced prostate cancer, multiple myeloma, pancreatic cancer, colorectal cancer (especially metastatic colorectal cancer) and breast cancer (especially triple negative breast cancer).

Prostate cancer is of particular interest as potential therapeutic target for IKKα inhibitors. Without wishing to be bound by any particular theory, in prostate cancer, the effective targeting of IKKα may enhance androgen deprivation therapy (ADT) chemotherapy responses by concurrently inhibiting androgen-driven and androgen-independent AR (androgen receptor) activity. IKKα inhibition may also abrogate inflammatory microenvironment signalling and eliminate tumour-promoting stimuli from adjacent stroma and infiltrating monocytes. IKKα inhibitors therefore have the potential to alter disease course, restore/prolong sensitivity to AR-targeted therapy and improve survival. Moreover, their use in hormone-sensitive de novo metastatic disease may significantly extend the benefit duration of conventional therapies and reduce the overall incidence of castration-resistant prostate cancer (CRPC). As a consequence, a IKKα inhibitor may find use in clinical scenarios such as:
the last-line therapy in patients with CRPC that have failed standard-of-care treatment
combination therapy with ADT to prevent the emergence of CRPC/prolong sensitivity to ADT
combination therapy in CRPC patients to restore sensitivity to ADT/reduce resistance development to chemotherapy
single-agent therapy to prevent the emergence of CRPC

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery or radiotherapy or chemotherapy or a targeted agent. Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5a-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, P13 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034).
(vi) vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, A2a receptor antagonists, P13 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

The compounds of the present invention are anticipated to be particularly useful in combination with androgen deprivation therapies (ADTs) and standard chemotherapy used to treat prostate cancer and, in particular, castrate-resistant prostate cancer (CRPC).

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

### EXAMPLES

### Experimental Section

General Methods. Unless otherwise stated, commercially available materials were used without further purification. Air- or moisture-sensitive reactions were carried out under a nitrogen atmosphere. Anhydrous solvents were obtained from Sigma-Aldrich. Flash chromatography was performed using silica gel under standard techniques¹ (Acros, 60 Å, 35-70 μm) or using a Biotage S P4 automated chromatography system (SNAP KP-Sil, 60 Å, 40-63 μm cartridges; detection wavelength: 254 nm; monitoring: 280 nm). NMR spectra (¹H and ¹³C) were recorded on either a JEOL ECX-400 (400 MHz); Bruker Avance3/DPX400 (400 MHz) or Bruker Avance/DPX500 (500 MHz) instruments. Chemical shifts (6) are quoted in parts per million (ppm) relative to an internal solvent reference. Coupling constants (J) are recorded in Hertz. Low resolution mass spectroscopy was carried out on ThermoFinnigan LCQ Duo by direct infusion, high resolution mass spectroscopy was carried out on a Exactive (thermo scientific) LCMS mass spectrometer. Reverse phase HPLC purifications were conducted on a Water HPLC system comprising a Waters 1525 binary HPLC pump, Waters 717 autosampler, Waters 2487 dual λ absorbance detector (254 nm), using a semi-preparative (50×21.2 mm) Luna 5μ C18 column(eluting with an acetonitrile/water gradient with 0.1% TFA in each solvent using the following gradient;

| Time | Flow rate | % Water 0.1% | % Acetonitrile 0.1% |
|---|---|---|---|
| (min) | (ml/min) | TFA | TFA |
| 0 | 6 | 90 | 10 |
| 25 | 6 | 50 | 50 |
| 30 | 6 | 30 | 70 |
| 35 | 6 | 90 | 10 |
| 40 | 0 | 90 | 10 |

Microwave reactions were carried out using a Biotage Initiator-8 Microwave synthesiser (operating at 2.45 GHz). Thin-layer chromatography (TLC) was carried out on aluminium-backed SiO₂ plates (Merck, silica gel 60, F₂₅₄) and spots visualised using ultra-violet light (254 nm) or by staining with potassium permanganate. All tested compounds were determined to be ≥95% purity by LC-MS and analytical HPLC unless otherwise stated.

### General Procedures

All commercially available reagents and solvents used were obtained from Sigma-Aldrich, Fluorochem Fisher Scientific, Acros, Alfa Aesar, Apollo scientific and Advanced ChemBlocks and used without further purification. Air- or moisture-sensitive reactions were carried out under argon or nitrogen atmosphere.

Microwave reactions were carried out using a Biotage Initiator system.

Flash chromatography was performed using a Biotage SP4 automated chromatography system using silica stationary phase (Fisher Scientific, 60 Å, 35-70 micron; detection wavelength: 254 nm; monitoring: 280 nm) and the mobile phase used are detailed in the text.

Reverse phase HPLC purifications were conducted on Shimadzu Prominance HPLC using a semi-preparative (50×21.2 mm) Luna 5 μm C18 column at 40° C.; flow rate: 6 ml/min; detection wavelength: 254 nm eluting with an acetonitrile/water gradient with 0.1% TFA.

NMR spectra were recorded on either a Bruker Avance3/DPX400 (400 MHz), Bruker DRX500 (500 MHz), Bruker AV400 (400 MHz), Bruker AV500HD (500 MHz) or Bruker AV600 (600 MHz) instrument and analysed using Advanced Chemistry Development Labs (ACD/labs) NMR processor 12.00 or MestReNova 10.0 software. Chemical shifts (6) are recorded in parts per million (ppm) relative to an internal solvent reference (tetramethylsilane) and coupling constants (J) in Hertz (Hz). Splitting patterns were indicated as singlet (s), broad singlet (br. s), doublet (d), doublet of doublet (dd), triplet (t), quartet (q) and multiplet (m).

LCMS was carried out on an Agilent Technologies 1220 series LC system with Agilent 6100 series quadrupole mass spectrometer in ESI/APCI mode. Separation was achieved with an Agilent Eclipse C18 4.6×50 mm column; flow rate: 1 ml/min; detection: 254 nm; sample volume: 10 μl; mobile phase: acetonitrile/5 mM ammonium acetate:water/5 mM ammonium acetate; 5%, 1.48 min; 5-100%, 8 min; 100%, 13.5 min; 100-5%, 16.5 min; 18 min. HRMS was carried out on an Exactive (Thermo scientific) or LTQ orbitrap (Thermo scientific).

### Section 1—Compounds of the Formula

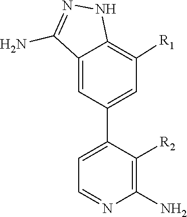

### Example 1

### 3-Chloro-2-fluoro-5-(4,4,5,5-tetramethyl-1,3,2-dioxaborolan-2-yl)benzonitrile

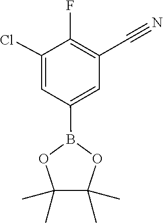 [SMILES: CC1(C)OB(c2cc(Cl)c(F)c(C#N)c2)OC1(C)C]

From a premade 25 mL catalyst solution of [Ir(OMe)cod]₂ (104 mg, 0.312 mmol [Ir]), bispinacolatodiborane (2644 mg, 10.4 mmol) and 4,4′-ditert-butyl-2,2′-bipyridine (84 mg, 0.312 mmol) dissolved in tert-butylmethylether was taken 7.2 mL which was added to a 10-20 mL microwave vial. To this was added 3-chloro-2-fluorobenzonitrile (934 mg, 6 mmol) and the solution was heated to 90° C. for 90 minutes under microwave conditions. Solution was filtered through celite using 10:1 CH₂Cl₂:methanol and taken forward without further purification.

### 5-(4,4,5,5-tetramethyl-1,3,2-dioxaborolan-2-yl)-7-chloro-3-amino-1H-indazole

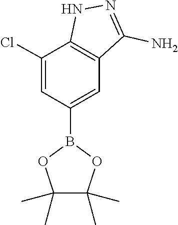 [SMILES: CC1(C)OB(c2cc(Cl)c3[nH]nc(N)c3c2)OC1(C)C]

A crude mixture of 3-chloro-2-fluoro-5-(4,4,5,5-tetramethyl-1,3,2-dioxaborolan-2-yl)benzonitrile was diluted in ethanol (20 mL) to which hydrazine hydrate (50-60%, 1.46 mL, 30 mmol) was added and the solution stirred at 90° C. for 28 hours. Water (75 mL) was added and the resulting precipitate filtered. Solids were washed with water (20×10 mL) and pet. ether 60-80° C. (10×10 mL) and dried under reduced pressure to yield a brown solid (678 mg, 39% over 2 steps). ¹H (DMSO-d₆, 400 MHz) δ 1.30 (s, 12H), 5.69 (br s, 2H), 7.46 (s, 1H), 8.17 (s, 1H), 12.02 (br s, 1H); ¹³C (DMSO-d₆, 100 MHz) δ 25.15 (4C), 84.03 (2C), 114.27, 116.70, 118.51, 128.10, 130.61, 140.38, 151.12; LR-MS ESI 294.13 (100%), 296.07 (32.6%).

### 5-(2-Aminopyridin-4-yl)-7-chloro-1H-indazol-3-amine

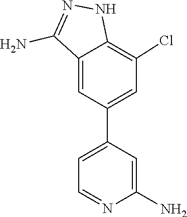 [SMILES: Nc1cc(-c2cc(Cl)c3[nH]nc(N)c3c2)ccn1]

5-(4,4,5,5-Tetramethyl-1,3,2-dioxaborolan-2-yl)-7-chloro-3-amino-1H-indazole (176.1 mg, 0.6 mmol), 2-amino-4-bromopyridine (86.5 mg, 0.5 mmol) and [1,1′-bis(di-tert-butylphosphino)ferrocene]dichloropalladium(II) catalyst (16.3 mg, 0.025 mmol) were placed in a 2-5 mL microwave vial which was sealed and purged with nitrogen. Ethanol (1.5 mL) was added and the suspension brought to 90° C. with stirring before addition of K₃PO₄ (O₂-free, 1 M, 0.75 mL). Stirring continued for 24 hours. Resultant solution was diluted in EtOAc, washed with water and adsorbed onto silica under reduced pressure. Chromatographic purification (Biotage SP4, 50 g cartridge, solvent system: EtOAc/methanol, 0%, 6 CV; 0-2%, 1 CV; 2%, 3 CV; 2-10%, 1CV; 10%, 4 CV) yielded product as a yellow solid (42 mg, 32%). ¹H (DMSO-d₆, 400 MHz) δ 5.65 (s, 2H), 5.92 (s, 2H), 6.71 (s, 1H), 6.79 (dd, J=5.4 & 1.1 Hz, 1H), 7.57 (s, 1H), 7.95 (d, J=5.6 Hz, 1H), 8.07 (s, 1H), 12.01 (br s, 1H); ¹³C (DMSO-d₆, 100 MHz) δ 104.62, 109.89, 114.66, 116.36, 117.67, 123.89, 129.11, 138.48, 147.67, 148.35, 150.63, 160.39; LR-MS ESI 260.13 (100%), 262.13 (33.7%). HRMS (ESI+ve): For C₁₂H₁₁N₅Cl requires 260.0697 found 260.0696 (100%) & 262.0665 (32%).

### Example 2

### (E)-5-Bromo-2-fluoro-3-methylbenzaldehyde oxime

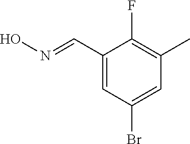

5-Bromo-2-fluoro-3-methylbenzaldehyde (2.00 g, 9.30 mmol) was dissolved in ethanol: water (1:2) (180 mL) along with hydroxylamine hydrochloride (1.26 g, 18.12 mmol) and 6 mL of a 50% w/w sodium hydroxide solution. The solution was then allowed to stir for 1 h at room temperature. The solution was then neutralised with conc. HCl to pH 7. The solution was then extracted with dichloromethane (150 mL) and the organic layer was then washed with brine (50 mL) and dried using magnesium sulfate. The solvent was then removed to afford the title compound as fine white needles (1.92 g, 8.35 mmol, 90% yield). ¹H NMR (500 MHz DMSO-d₆): δ 2.23 (s, 3H), 7.52 (dd, J=6.5, 2.0 Hz, 1H), 7.64 (dd, J=6.0, 2.5 Hz, 1H), 8.16 (s, 1H), 11.74 (s, 1H). LRMS: For C₃H₇BrFNO requires 231.96 found 231.98 (M+H).

### 5-Bromo-2-fluoro-3-methylbenzonitrile

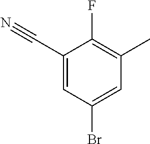

(E)-5-Bromo-2-fluoro-3-methylbenzaldehyde oxime (1.90 g, 8.26 mmol) was dissolved in acetic anhydride (10 mL) and the solution heated to 140° C. for 3 h. The solution was then allowed to cool and the solvent removed under high vacuum to afford the desired product as pale brown needles (1.55 g, 7.31 mmol, 86%). ¹H NMR (500 MHz DMSO-d₆): δ 2.28 (d, J=2.0 Hz, 3H), 7.93 (m, 1H), 8.04 (m, 1H), LRMS: For C₃H₅BrFN requires 212.96 found 213.97 (M+H).

### 5-(2-Aminopyridin-4-yl)-7-methyl-1H-indazol-3-amine

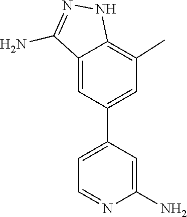 [SMILES: Cc1cc(-c2ccnc(N)c2)cc2c(N)n[nH]c12]

4-(4,4,5,5-Tetramethyl-1,3,2-dioxaborolan-2-yl)pyridin-2-amine (0.352 g, 0.80 mmol), 5-bromo-2-fluoro-3-methylbenzonitrile (0.085 g, 0.40 mmol) and sodium acetate (0.066 g, 0.80 mmol) were dissolved in IPA: Water (2: 1) (5 mL) in a 10 mL microwave vial, and the solution degassed with N₂ for 15 min. [1,1′-bis(diphenylphosphino)ferrocene]dichloropalladium(II) catalyst (0.040 g) was then added and the vial sealed before heating to 160° C. for 40 min in the microwave. Hyrdazine hydrate (1 mL) was then added and the solution heated to 100° C. for a further 1 hr. The solvent was then removed under reduced pressure and the residue dissolved in DMF (1 mL) and purified by HPLC to give the desired product as a white solid (0.0054 g, 0.023 mmol, 6%). ¹H NMR (500 MHz DMSO-d₆): δ 2.47 (s, 3H), 7.21 (m, 2H), 7.45 (s, 1H), 7.93 (s, 2H), 7.99 (d, J=7.2 Hz, 1H), 8.14 (d, J=1.2 Hz, 1H), 12.10 (s, 1H) LRMS: For C₁₃H₁₃N₅ requires 239.12 found 240.13 (M+H).

### Example 3

### 5-Bromo-2-fluoro-3-(trifluoromethyl)benzonitrile

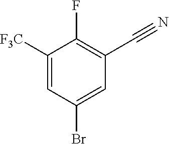 [SMILES: N#Cc1cc(Br)cc(C(F)(F)F)c1F]

5-Bromo-2-fluoro-3-(trifluoromethyl)benzaldehyde oxime (1.5 g, 5.25 mmol) was solubilised in acetic anhydride (10 mL) and shared equally between two 2-5 mL microwave vials which were sealed and purged with nitrogen. Vials were subject to microwave irradiation for 2 hours at 180° C. Resultant solutions were combined, diluted in EtOAc (100 mL) and washed with saturated sodium carbonate (100 mL). Organics were dried over magnesium sulfate and adsorbed onto silica under reduced pressure. Chromatographic purification (Biotage SP4, 50 g cartridge, solvent system: pet. ether/EtOAc, isocratic, 5%, 8 CV) yielded title product as a white waxy solid (920 mg, 65%). (¹H (DMSO-d₆, 500 MHz) δ 8.40 (dd, J=6.3 & 2.2 Hz, 1H), 8.64 (dd, J=5.3 & 2.5 Hz, 1H); ¹³C (DMSO-d₆, 125 MHz) δ 105.00 (d, J=15.6 Hz), 112.16, 117.70 (d, J=3.7 Hz), 119.67 (dd, J=34.1 & 11.1 Hz), 121.43 (d, J=273.9 Hz), 135.80 (d, J=3.7 Hz), 141.23, 159.51 (d, J=265.6 Hz).

### 5-Bromo-7-(trifluoromethyl)-3-amino-1H-indazole

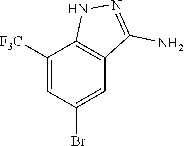 [SMILES: Nc1n[nH]c2c(C(F)(F)F)cc(Br)cc12]

5-Bromo-2-fluoro-3-(trifluoromethyl)benzonitrile (900 mg, 3.36 mmol), sodium bicarbonate (378 mg, 4.5 mmol) and ethanol (4 mL) were placed in a 10-20 mL microwave vial, sealed and purged with nitrogen. Solution was heated to 90° C. with stirring at which point hydrazine hydrate (0.73 mL, 15 mmol) was added. Solution was stirred for 18 hours then cooled to room temperature. Water (15 mL) was added and precipitate filtered, washed with water (10×10 mL) and pet. ether (5×10 mL) to tield title product as a crystalline white solid (840 mg, 89%). ¹H (DMSO-d₆, 500 MHz) δ 5.72 (s, 2H), 7.69 (s, 1H), 8.28 (s, 1H), 12.17 (s, 1H); ¹³C (DMSO-d₆, 125 MHz) δ 107.97, 112.40 (d, J=34.8 Hz), 118.28, 126.61 (d, J=4.6 Hz), 128.26 134.98, 149.73.

### 5-(4,4,5,5-Tetramethyl-1,3,2-dioxaborolan-2-yl)-7-(trifluoromethyl)-3-amino-1H-indazole

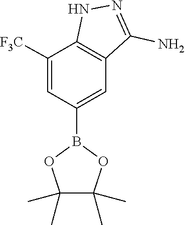 [SMILES: CC1(C)OB(c2cc(C(F)(F)F)c3[nH]nc(N)c3c2)OC1(C)C]

5-Bromo-7-(trifluoromethyl)-3-amino-1H-indazole (280 mg, 1 mmol), bis(pinacolato)diborane (280 mg, 1.1 mmol), [1,1′-bis(diphenylphosphino)ferrocene]dichloropalladium(II) catalyst (36.6 mg, 0.05 mmol) and 1,4-dioxane (5 mL) were placed in a 10-20 mL microwave vial which was sealed and purged with nitrogen. Potassium acetate (264 mg, 3 mmol) in water (0.5 mL) was added and the solution stirred at 100° C. for 24 hours. Resultant solution was suspended in EtOAc (150 mL) and methanol (10 mL) and washed with water (2×50 mL) and brine (50 mL). Organics were dried over magnesium sulfate and adsorbed onto silica under reduced pressure. Chromatographic purification (Biotage SP4, 50 g cartridge, solvent system: pet. ether/EtOAc, 30%, 5 CV; 30-70%, 1 CV; 70%, 3 CV) yielded title product as a brown solid (190 mg, 58%). ¹H (DMSO-d₆, 500 MHz) δ 1.31 (s, 12H), 5.80 (s, 2H), 7.72 (s, 1H), 8.45 (s, 1H), 12.10 (s, 1H);

### 5-(2-Aminopyridin-4-yl)-7-(trifluoromethyl)-1H-indazol-3-amine

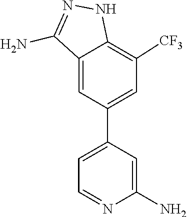 [SMILES: Nc1cc(-c2cc(C(F)(F)F)c3[nH]nc(N)c3c2)ccn1]

2-Amino-4-chloropyridine (128 mg, 1 mmol), [1,1′-bis(di-tert-butylphosphino)ferrocene]dichloropalladium(II) catalyst (12 mg, 0.019 mmol) and 5-(4,4,5,5-tetramethyl-1,3,2-dioxaborolan-2-yl)-7-(trifluoromethyl)-3-amino-1H-indazole (120 mg, 0.37 mmol) dissolved in ethanol (O₂-free, 0.66 mL) were placed in a 2-5 mL microwave vial which was sealed and purged with nitrogen. Further ethanol (O₂-free, 0.84 mL) was added and the solution heated to 100° C. at which point K₃PO₄ (1 M, 0.74 mL) was added and the solution stirred for 24 hours. Solution was cooled, suspended in EtOAc (100 mL) and washed with water (2×50 mL) and brine (50 mL). Organics were dried over magnesium sulfate and adsorbed onto silica under reduced pressure. Chromatographic purification (Biotage SP4, 50 g cartridge, solvent system: EtOAc/methanol, 0%, 4 CV; 0-2%, 3 CV; 2%, 2 CV; 2-10%, 1CV; 10%, 4 CV) yielded product as a yellowish solid (47 mg, 44%). ¹H (DMSO-d₆, 500 MHz) δ 5.77 (s, 2H), 5.96 (s, 2H), 6.75 (d, J=0.9 Hz, 1H), 6.75 (dd, J=5.3 & 1.6 Hz, 1H), 7.79 (s, 1H), 7.97 (d, J=5.3 Hz, 1H), 8.40 (s, 1H), 12.09 (s, 1H); ¹⁹F (DMSO-d₆, 376 MHz) δ −60.34; ¹³C (DMSO-d₆, 100 MHz) δ 105.09, 110.26, 117.43, 119.28 (d, J=245.8 Hz), 122.72 (d, J=4.4 Hz), 123.21, 123.88, 125.90, 127.84, 147.87, 149.00, 150.88, 160.97; HRMS (ESI+ve): For C₁₃H˜F₃N₅ requires 294.0961 found 294.0961.

### Section 2—Compounds of the Formula

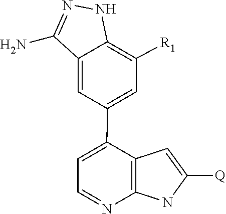

### Example 4

### 7-Chloro-5-(1H-pyrrolo[2,3-b]pyridin-4-yl)-1H-indazol-3-amine

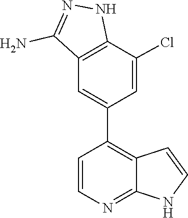 [SMILES: Nc1n[nH]c2c(Cl)cc(-c3ccnc4[nH]ccc34)cc12]

4-Bromo-1H-pyrrolo[2,3-b]pyridine (0.291 g, 1.50 mmol) along with 7-chloro-5-(4,4,5,5-tetramethyl-1,3,2-dioxaborolan-2-yl)-1H-indazol-3-amine (0.502 g, 1.5 mmol), dioxane (8 ml) and potassium phosphate (4 ml of 1M solution)) were placed in a 20 ml ml microwave vial. The solution was then degassed with N₂ for 10 min. [1,1′-bis(di-tert-butylphosphino)ferrocene]dichloropalladium(II) catalyst (118) (0.118 g, 0.17 mmol, 5 mol %) was then added and the vial sealed and heated to 90° C. for 16 hrs. The solution was then allowed to cool before removing the solvent under reduced pressure to give the crude product which was purified by flash chromatography to give the desired product. (321 mg, 0.98 mmol, 65%), δ_{H} (d₆-DMSO): 5.57 (s, 1H), 5.69 (s, 2H), 6.70 (q, J=1.72 1H), 7.21 (d, J=5.00, 1H) 7.56 (t, J=2.76, 1H), 7.84 (d, J=1.24, 1H), 8.22 (d, J=1.28 Hz, 1H), 8.27 (d, J=5.00 Hz, 1H), 11.76 (s, 1H), 11.96 (s, 1H). LRMS: found: 283.2 (M+1), calculated C₁₄H₁₀ClN: 284.2.

### Example 5

### 7-Bromo-5-(1H-pyrrolo[2,3-b]pyridin-4-yl)-1H-indazol-3-amine

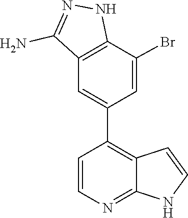 [SMILES: Nc1n[nH]c2c(Br)cc(-c3ccnc4[nH]ccc34)cc12]

4-Bromo-1H-pyrrolo[2,3-b]pyridine (0.291 g, 1.50 mmol) along with 7-bromo-5-(4,4,5,5-tetramethyl-1,3,2-dioxaborolan-2-yl)-1H-indazol-3-amine (0.502 g, 1.5 mmol), dioxane (8 ml) and potassium phosphate (4 ml of 1M solution)) were placed in a 20 ml ml microwave vial. The solution was then degassed with N₂ for 10 min. [1,1′-bis(di-tert-butylphosphino)ferrocene]dichloropalladium(II) catalyst (0.118 g, 0.17 mmol, 5 mol %) was then added and the vial sealed and heated to 90° C. for 16 hrs. The solution was then allowed to cool before removing the solvent under reduced pressure to give the crude product which was purified by flash chromatography to give the desired product. (321 mg, 0.98 mmol, 65%), δ_{H}(d₆-DMSO): 5.57 (s, 1H), 5.69 (s, 2H), 6.70 (q, J=1.72 1H), 7.21 (d, J=5.00, 1H) 7.56 (t, J=2.76, 1H), 7.84 (d, J=1.24, 1H), 8.22 (d, J=1.28 Hz, 1H), 8.27 (d, J=5.00 Hz, 1H), 11.76 (s, 1H), 11.96 (s, 1H). LRMS: found: 328.17 (M+1), calculated C₁₄H₁₀BrN₅: 327.01.

### Example 6

### 7-Ethynyl-5-(1H-pyrrolo[2,3-b]pyridin-4-yl)-1H-indazol-3-amine

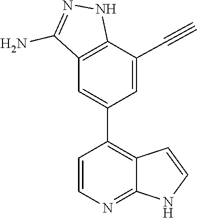 [SMILES: C#Cc1cc(-c2ccnc3[nH]ccc23)cc2c(N)n[nH]c12]

7-Bromo-5-(1H-pyrrolo[2,3-b]pyridin-4-yl)-1H-indazol-3-amine (0.200 g, 0.61 mmol) and ethynyltrimethylsilane (0.169 ml, 1.2 mmol) were added to a sealable vial containing triethylamine (2 ml) and DMF (2 ml) and the solution degassed with N₂ for 10 min. [1,1′-bis(diphenylphosphino)ferrocene]dichloropalladium(II) catalyst (0.064 g, 0.08 mmol), and CuI (0.017 g, 0.08 mmol) were added and the vial sealed and heated to 60° C. for 16 hrs. The solution was then allowed to cool before removing the solvent under reduced pressure to give the crude product which was purified by flash chromatography to give the desired product. (64 mg, 0.23 mmol, 38%), δ_{H} (d₆-DMSO): 5.10 (s, 1H), 5.66 (s, 2H), 6.69 (q, J=1.48 1H), 7.20 (d, J=4.76, 1H) 7.55 (t, J=3.28, 1H), 7.75 (d, J=1.48, 1H), 8.25 (m, 2H), 11.76 (s, 1H), 11.96 (s, 1H). LRMS: found: 274.2 (M+1), calculated C₁₄H₁₀BrN₅: 273.1.

### Example 7

### 7-Phenyl-5-(1H-pyrrolo[2,3-b]pyridin-4-yl)-1H-indazol-3-amine

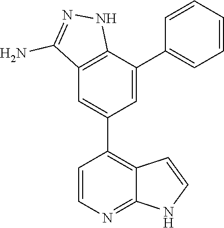 [SMILES: Nc1n[nH]c2c(-c3ccccc3)cc(-c3ccnc4[nH]ccc34)cc12]

A 2-5 mL MW tube was charged with a mixture of 7-chloro-5-(1H-pyrrolo[2,3-b]pyridin-4-yl)-1H-indazol-3-amine (Example 4) (57 mg, 0.2 mmol, 1 eq.), phenylboronic acid (49 mg, 1.2 eq., 2 eq.), [1,1′-bis(di-tert-butylphosphino)ferrocene]dichloropalladium(II) catalyst (6.5 mg, 5 mol %) in 1,4-dioxane (1.2 mL) was stirred under argon before adding 1 M aq. K₃PO₄ (0.6 mL, 3 eq.). The reaction was heated to 50° C. under a gentle flow of argon for 10 minutes, then to 110° C. for 17 hours during which time the initial orange solution became a suspension. The stirred reaction mixture was then cooled to room temperature and slowly diluted with water (15 mL) and filtered. The filtered solid was washed with water (3×5 mL) and hexane (3×5 mL) to give a crude product (63 mg). Purification by flash chromatography (80-100% AcOEt in hexane) gave the title compound as a beige solid (19 mg). ¹H NMR (400 MHz, DMSO-D₆) δ 5.60 (s, 2H), 6.76 (dd, J=3.5, 1.8 Hz, 1H), 7.28 (d, J=5.1 Hz, 1H), 7.39-7.47 (m, 1H), 7.49-7.58 (m, 3H), 7.69 (d, J=1.7 Hz, 1H), 7.75-7.81 (m, 2H), 8.20 (d, J=1.6 Hz, 1H), 8.27 (d, J=5.1 Hz, 1H), 11.71 (s, 1H), 11.74 (s, 1H).

### Example 8

### 5-(2-Methyl-1H-pyrrolo[2,3-b]pyridin-4-yl)-1H-indazol-3-amine

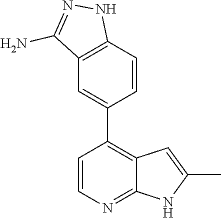 [SMILES: Cc1cc2c(-c3ccc4[nH]nc(N)c4c3)ccnc2[nH]1]

A suspension of 4-chloro-2-methyl-7-azaindole (0.058 g, 0.35 mmol), 5-(4,4,5,5-tetramethyl-1,3,2-dioxaborolan-2-yl)-1H-indazol-3-amine (0.136 g, 0.53 mmol), 1M potassium phosphate solution (0.87 ml, 0.87 mmol), [1,1′-bis(di-tert-butylphosphino)ferrocene]dichloropalladium(II) catalyst (0.011 g, 0.0175 mmol) in 1.5 ml ethanol/water (1:1) was deoxygenated with nitrogen in sealed tube and the mixture allowed to stir at 90-100° C. for 18 h. After the reaction was cooled to room temperature, EtOAc and water were added. The extracted organic layer was dried over magnesium sulfate and concentrated under reduced pressure and the residue purified by column chromatography (80-90% EtOAc and 1% triethylamine in petroleum ether 60-80%) to give the titled compound as a white solid (35 mg, 38%), ¹H NMR (400 MHz, DMSO-d₆) δ ppm 2.63 (s, 3H) 5.53 (s, 2H) 6.37 (d, J=2.20 Hz, 1H) 7.12 (d, J=4.83 Hz, 1H) 7.37 (d, J=8.35 Hz, 1H) 7.62 (dd, J=8.79, 1.76 Hz, 1H) 8.12 (s, 1H) 8.17 (d, J=5.27 Hz, 1H) 11.53 (s, 1H) 11.62 (s, 1H). m/z (ESI-HRMS) calculated for C₁₅H₁₄N₅(M+H⁺) 264.1249 found=264.1253 (M+H⁺)

### Example 9

### 2-(tert-Butyl)-7-oxide-7-azaindole

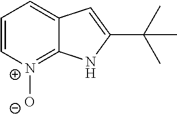

To an ice-cooled solution of 2-tert-butyl azaindole, (0.1 g, 0.57 mmol) in EtOAc, meta-chloroperoxybenzoic acid (0.16 g, 0.91 mmol) was added slowly. Then, the reaction was warmed to room temperature and stirred to 1 h. After the reaction was completed, the solvent was evaporated, treated, with 1M sodium carbonate solution, and extracted with EtOAc. The residue was then concentrated under vacuum to give the product as a yellow solid (68.4 mg, 63%). ¹H NMR (400 MHz, DMSO-d₆) δ ppm 1.36 (s, 9H) 6.26 (d, J=1.76 Hz, 1H) 7.01 (dd, J=7.91, 6.15 Hz, 1H) 7.51 (d, J=7.91 Hz, 1H) 8.04 (d, J=6.15 Hz, 1H) 12.25 (br. s., 1H). ¹³C NMR (100 MHz, DMSO-d₆) δ ppm 29.93, 32.67, 97.14, 116.54, 119.27, 124.58, 130.97, 139.38, 151.47. m/z (ESI-MS) [M]⁺ 191.1.

### 2-(Tert-butyl)-4-(3-amino-1H-indazol-5-yl)-7-azaindole

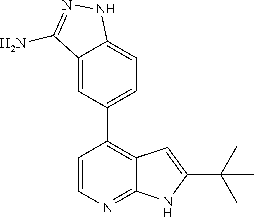 [SMILES: CC(C)(C)c1cc2c(-c3ccc4[nH]nc(N)c4c3)ccnc2[nH]1]

A suspension of 2-(tert-butyl)-4-chloro-7-azaindole (0.052 g, 0.28 mmol), 5-(4,4,5,5-tetramethyl-1,3,2-dioxaborolan-2-yl)-1H-indazol-3-amine (0.096 g, 0.37 mmol), 1M potassium phosphate solution (0.49 ml, 0.49 mmol), [1,1′-bis(di-tert-butylphosphino)ferrocene]dichloropalladium(II) catalyst (0.008 g, 0.0124 mmol) in 1.4 ml ethanol was deoxygenated with nitrogen in sealed tube and the mixture allowed to stir at 90-100° C. for 18 h. After the reaction was cooled to room temperature, EtOAc and water were added. The extracted organic layer was dried over magnesium sulfate and concentrated under reduced pressure and the residue purified by column chromatography (80-90% EtOAc and 1% triethylamine in petroleum ether 60-80%) to give the titled compound as a white solid (33.5 mg, 44%). ¹H NMR (400 MHz, DMSO-d₆) b ppm 1.38 (s, 9H) 5.52 (s, 2H) 6.35 (d, J=2.20 Hz, 1H) 7.10 (d, J=4.83 Hz, 1H) 7.38 (d, J=8.35 Hz, 1H) 7.63 (dd, J=8.79, 1.76 Hz, 1H) 8.12 (s, 1H) 8.17 (d, J=5.27 Hz, 1H) 11.53 (s, 1H) 11.62 (s, 1H). ¹³C NMR (100 MHz, DMSO-d₆) 30.21, 32.51, 94.00, 110.37, 114.49, 115.12, 118.15, 120.67, 127.23, 128.74, 140.07, 140.93, 141.62, 142.34, 150.40, 150.62. m/z (ESI-HRMS) calculated for C₁₈H₂₀N₅=306.1713 found=306.1710.

### Example 10

### 4-(3-Amino-1H-indazol-5-yl)-1H-pyrrolo[2,3-b]pyridine-2-carboxylic acid

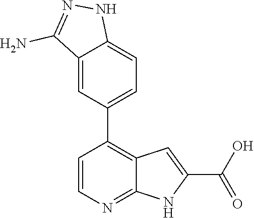 [SMILES: Nc1n[nH]c2ccc(-c3ccnc4[nH]c(C(=O)O)cc34)cc12]

The purification by HPLC of the reaction for the synthesis of 4-(3-amino-1H-indazol-5-yl)-N-isopentyl-1H-pyrrolo[2,3-b]pyridine-2-carboxamide (Example 237) afforded also the title compound as a yellow solid (0.0091 g, 0.0175 mmol, 22%). ¹H NMR (400 MHz, DMSO-d₆) δ 7.27 (d, J=5.0 Hz, 1H), 7.34 (s, 1H), 7.43 (d, J=8.8 Hz, 1H), 7.70 (dd, J=8.7, 1.6 Hz, 1H), 8.24 (s, 1H), 8.43 (d, J=5.0 Hz, 1H), 12.45 (s, 1H). HRMS: Calculated for C₁₅H₁₂O₂N₅ (M+H⁺)=294.0986; Found: 294.0987

### Section 3—Compounds of the Formula

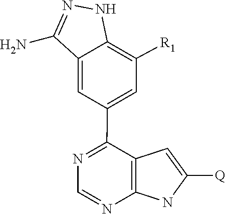

### Example 11

### 7-Bromo-5-(7H-pyrrolo[2,3-d]pyrimidin-4-yl)-1H-indazol-3-amine

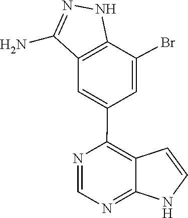 [SMILES: Nc1n[nH]c2c(Br)cc(-c3ncnc4[nH]ccc34)cc12]

4-Bromo-7H-pyrrolo[2,3-d]pyrimidine (0.200 g, 1.02 mmol) along with 7-bromo-5-(4,4,5,5-tetramethyl-1,3,2-dioxaborolan-2-yl)-1H-indazol-3-amine (0.401 g, 1.20 mmol), dioxane (6 ml) and potassium phosphate (3 ml of 1M solution)) were placed in a 20 ml ml microwave vial. The solution was then degassed with N₂ for 10 min. [1,1′-bis(di-tert-butylphosphino)ferrocene]dichloropalladium(II) catalyst (0.118 g, 0.17 mmol) was then added and the vial sealed and heated to 90° C. for 16 hrs. The solution was then allowed to cool before removing the solvent under reduced pressure to give the crude product which was purified by flash chromatography to give the desired product. (180 mg, 0.55 mmol, 53%), δ_{H}(d₆-DMSO): 5.85 (s, 2H), 7.14 (d, J=3.5, 1H) 7.67 (d, J=2.90, 1H), 8.40 (s, 1H), 8.67 (s, 1H), 8.79 (s, 1H), 12.07 (s, 1H), 12.22 (s, 1H). LRMS: found: 329.15 (M+1), calculated C₁₃H₉BrN₆: 328.01.

### Section 4—Compounds of the Formula

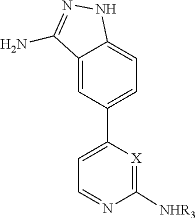

### Example 12

### 5-[2-(Ethylamino)pyridin-4-yl]-1H-indazol-3-amine

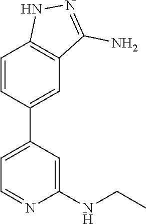 [SMILES: CCNc1cc(-c2ccc3[nH]nc(N)c3c2)ccn1]

A suspension of 4-chloro-N-ethylpyridin-2-amine (0.179 g, 1.15 mmol), 3-cyano-4-fluorophenylboronic acid (0.446 g, 1.81 mmol), [1,1′-bis(di-tert-butylphosphino)ferrocene]dichloropalladium(II) catalyst (0.062 g, 0.095 mmol) in IPA/H₂O (3:1.5 mL) was degassed with nitrogen. t-Butylamine (0.60 mL, 5.71 mmol) was then added and degassed using nitrogen. The reaction was placed in a microwave and irradiated at 160° C. for 40 min. Once cooled to room temperature the reaction mixture was concentrated under reduced pressure then suspended in EtOH (4.5 mL). To this suspension was added hydrazine hydrate (0.30 mL, 5.71 mmol) and the reaction mixture was placed in a microwave and irradiated at 165° C. for 30 min. The reaction mixture was diluted with MeOH and concentrated under reduced pressure. Purification of the resulting solid was carried out using column chromatography (100% EtOAc to 20% MeOH/EtOAc). The resulting solid was triturated with Et₂O and hexane then filtered under reduced pressure to give the title compound as a pale yellow solid (0.075 g, 0.296 mmol). ¹H NMR (400 MHz, DMSO-d₆): δ 1.16 (t, 3H, J=7.2, 14.4 Hz), 3.29 (q, 2H, J=10.1 Hz), 5.48 (br s, 2H), 6.45 (t, 1H, J=5.6, 10.8 Hz), 6.68 (s, 1H), 6.76 (dd, 1H, J=1.2, 5.2 Hz), 7.30 (d, 1H, J=8.4 Hz), 7.52 (dd, 1H, J=1.6, 8.8 Hz), 8.00 (d, 1H, J=5.2 Hz), 8.07 (s, 1H), 11.51 (br s, 1H). HRMS: For C₁₄H₁₆N₅ requires 254.1400 found 254.1407.

### Example 13

### 5-(2-(Propylamino)pyridin-4-yl)-1H-indazol-3-amine

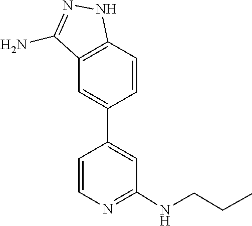 [SMILES: CCCNc1cc(-c2ccc3[nH]nc(N)c3c2)ccn1]

A suspension of 4-bromo-N-propylpyridin-2-amine (0.094 g, 0.44 mmol), 3-cyano-4-fluorophenylboronic acid (0.122 g, 0.54 mmol), t-butylamine (138 μL, 1.31 mmol), [1,1′-bis(di-tert-butylphosphino)ferrocene]dichloropalladium(II) catalyst (0.024 g, 0.04 mmol), iso-propanol (2 mL) and H₂O (1 mL) was degassed with nitrogen. The reaction was placed in a microwave and irradiated at 160° C. for 40 min. Once cooled to room temperature the reaction mixture was concentrated under reduced pressure then suspended in EtOH (2 mL). To this suspension was added hydrazine hydrate (57 μL, 1.17 mmol) and NaHCO₃ (0.030 g). The resulting suspension was heated to 80° C. for 2 d at which point additional hydrazine hydrate (28.5 μL, 2.5 eq.) and EtOH (1 mL) were added. Heating was continued for 1.5 d. The reaction mixture was then cooled down to 40° C., diluted with water (6 mL), stirred at room temperature for 30 min and left in the fridge overnight. The resulting precipitate was collected by filtration, washed with cold H₂O (2×20 mL) and 1:1 EtOH/H₂O (2×20 mL). The aqueous filtrates were combined and extracted with EtOAc (50 mL). The organic layer was combined to the initial precipitate, concentrated onto silica gel and purified by flash chromatography (Biotage SP4, 50 g SiO₄, EtOAc to remove the impurities, followed by 7% MeOH—1% triethylamine in EtOAc) to afford the title compound as a light yellow solid (0.028 g, 0.105 mmol, 45%). ¹H NMR (400 MHz, DMSO-d₆): δ 0.93 (t, J=7.4 Hz, 1H), 1.50-1.61 (m, 1H), 3.18-3.28 (m, 1H), 5.47 (s, 1H), 6.49 (t, J=5.7 Hz, 1H), 6.69 (s, 1H), 6.75 (dd, J=5.4, 1.6 Hz, 1H), 7.30 (d, J=8.8 Hz, 1H), 7.52 (dd, J=8.7, 1.9 Hz, 1H), 7.98 (d, J=5.4 Hz, 1H), 8.06 (s, 1H), 11.50 (br s, 1H).

### Example 14

### 5-(2-(Isopropylamino)pyridin-4-yl)-1H-indazol-3-amine

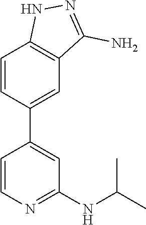 [SMILES: CC(C)Nc1cc(-c2ccc3[nH]nc(N)c3c2)ccn1]

A suspension of 4-bromo-N-isopropylpyridin-2-amine (0.050 g, 0.23 mmol), 3-cyano-4-fluorophenylboronic acid pinacol ester (0.115 g, 0.5 mmol), [1,1′-bis(di-tert-butylphosphino)ferrocene]dichloropalladium(II) catalyst (0.030 g, 0.05 mmol) in IPA/H₂O (2:1 mL) was degassed with nitrogen. t-Butylamine (0.25 mL, 2.32 mmol) was added and the mixture was heated in the microwave at 160° C. for 40 min. The reaction mixture was concentrated and taken up in EtOH (4 mL) and degassed with nitrogen. Hydrazine hydrate (0.34 mL, 7.0 mmol) was added and the mixture was heated in the microwave at 165° C. for 30 min. The reaction mixture was concentrated and purified by flash column chromatography (silica gel with EtOAc (50-100%) in hexane) to afford the title compound as an off-white solid. ¹H NMR (400 MHz, DMSO-d₆): 1.16 (s, 3H), 1.17 (s, 3H), 3.98-4.08 (m, 1H), 5.46 (br s, 2H), 6.30 (d, J=7.6 Hz, 1H), 6.67 (s, 1H), 6.74 (dd, J=5.4, 1.0 Hz, 1H), 7.30 (d, J=8.7 Hz, 1H), 7.51 (dd, J=8.7, 1.5 Hz, 1H), 7.98 (d, J=5.4 Hz, 1H), 8.05 (s, 1H), 11.50 (br s, 1H). HRMS: C₁₅H₁₈N₅ requires 268.1557, found 268.1553 (M+H)⁺.

### Example 15

### 5-(2-((Cycipropylmethyl)amino)pyridine-4-yl)-1H-indazol-3-amine

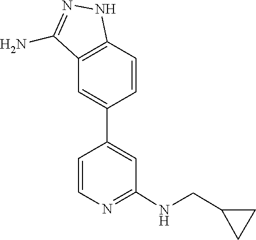 [SMILES: Nc1n[nH]c2ccc(-c3ccnc(NCC4CC4)c3)cc12]

A suspension of 4-bromo-N-(cyclopropylmethyl)pyridine-2-amine (0.020 g, 0.09 mmol), 3-cyano-4-fluorophenylboronic acid (0.04, 0.16 mmol), [1,1′-bis(di-tert-butylphosphino)ferrocene]dichloropalladium(II) catalyst (0.010 g, 0.02 mmol) in IPA/H₂O (3:1.5 mL) was degassed with nitrogen. t-Butylamine (0.25 mL, 2.38 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.20 mL, 5.00 mmol) and the mixture irradiated at 165° C. for 30 min. The reaction mixture was then concentrated under reduced pressure and purified using column chromatography (100% EtOAc) to give the desired product as a yellow solid (0.004 g, 0.02 mmol, 17%). ¹H NMR (400 MHz, DMSO-d₆): δ 0.22 (d, J=4.4 Hz, 2H), 0.44 (d, J=8.0 Hz, 2H), 1.07 (br s, 1H), 3.17 (t, J=5.8 Hz, 2H), 5.47 (br s, 2H), 6.57 (br s, 1H), 6.73 (s, 1H), 6.77 (d, J=5.2 Hz, 1H), 7.30 (d, J=8.8 Hz, 1H), 7.52 (d, J=8.8 Hz, 1H), 7.98 (d, J=4.8 Hz, 1H), 8.06 (s, 1H), 11.51 (br s, 1H).

### Example 16

### 5-(2-(Isopentylamino)pyridin-4-yl)-1H-indazol-3-amine

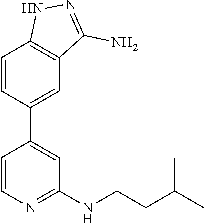 [SMILES: CC(C)CCNc1cc(-c2ccc3[nH]nc(N)c3c2)ccn1]

A suspension of 4-bromo-N-isopentylpyridin-2-amine (0.05 g, 0.23 mmol), 3-cyano-4-fluorophenylboronic acid pinacol ester (0.115 g, 0.50 mmol), [1,1′-bis(di-tert-butylphosphino)ferrocene]dichloropalladium(II) catalyst (0.030 g, 0.05 mmol) in IPA/H₂O (3:1.5 mL) was degassed with nitrogen. t-Butylamine (0.25 mL, 2.32 mmol) was added and the mixture was heated in the microwave at 160° C. for 40 min. The reaction mixture was concentrated and taken up in EtOH (4 mL) and degassed with nitrogen. Hydrazine hydrate (0.34 mL, 7.00 mmol) was added and the mixture was heated in the microwave at 165° C. for 30 min. The reaction mixture was concentrated and purified by flash column chromatography (silica gel with EtOAc (50-100%) in hexane) to afford the title compound as an off-white solid (0.011 g, 0.04 mmol, 16%). ¹H NMR (400 MHz, DMSO-d₆): 0.91 (s, 3H), 0.93 (s, 3H), 1.64-1.72 (m, 1H), 3.24-3.30 (m, 2H), 5.47 (br s, 2H), 6.42 (t, J=5.5 Hz, 1H), 6.68 (s, 1H), 6.76 (dd, J=4.7, 1.3 Hz, 1H), 7.30 (d, J=8.5 Hz, 1H), 7.52 (dd, J=7.3, 1.6 Hz, 1H), 7.99 (d, J=5.3, 1H), 8.06 (s, 1H), 11.50 (br s, 1H). HRMS: C₁₇H₂₂N₅ requires 296.1870, found 296.1867 (M+H)+.

### Example 17

### 5-(2-(Hexylamino)pyridine-4-yl)-1H-indazol-3-amine

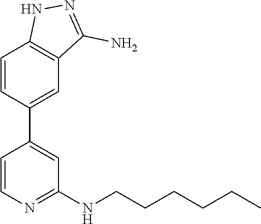 [SMILES: CCCCCCNc1cc(-c2ccc3[nH]nc(N)c3c2)ccn1]

A suspension of 4-bromo-N-hexylpyridin-2-amine (0.090 g, 0.35 mmol), 3-cyano-4-fluorophenylboronic acid (0.148 g, 0.60 mmol), [1,1′-bis(di-tert-butylphosphino)ferrocene]dichloropalladium(II) catalyst (0.028 g, 0.04 mmol) in IPA/H₂O (3:1.5 mL) was degassed with nitrogen. t-Butylamine (0.25 mL, 2.38 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 5.0 mmol) and the mixture irradiated at 165° C. for 30 min. The reaction mixture was then concentrated under reduced pressure and purified using column chromatography (100% Hexane—100% EtOAc). The resulting solid was triturated with diethyl ether, filtered and dried to give the title compound as an off-white solid (0.061 g, 0.20 mmol, 56.2%). ¹H NMR (400 MHz, DMSO-d₆): δ 0.88 (br s, 3H), 1.30-1.35 (m, 6H), 1.54 (br s, 2H), 3.26 (br s, 2H), 5.47 (br s, 2H), 6.46 (br s, 1H), 6.68 (br s, 1H), 6.75 (br s, 1H), 7.29-7.31 (m, 1H), 7.50-7.53 (m, 1H), 7.99 (m, 1H), 8.06 (s, 1H), 11.50 (br s, 1H). HRMS: For C₁₈H₂₄N₅ requires 310.2026 found 310.2023.

### Example 18

### 5-(2-(Cyclohexylamino)pyridine-4-yl)-1H-indazol-3-amine

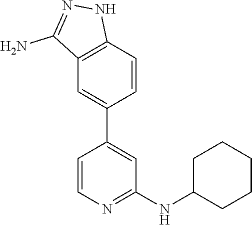 [SMILES: Nc1n[nH]c2ccc(-c3ccnc(NC4CCCCC4)c3)cc12]

A suspension of 4-bromo-N-cyclohexylpyridin-2-amine (0.108 g, 0.43 mmol), 3-cyano-4-fluorophenylboronic acid (0.168 g, 0.68 mmol), [1,1′-bis(di-tert-butylphosphino)ferrocene]dichloropalladium(II) catalyst (0.029 g, 0.04 mmol) in IPA/H₂O (3:1.5 mL) was degassed with nitrogen. t-Butylamine (0.25 mL, 2.38 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.25 mL, 5.5 mmol) and the mixture irradiated at 165° C. for 30 min. The reaction mixture was then concentrated under reduced pressure and purified using column chromatography (100% EtOAc). The resulting solid was triturated from diethyl ether, filtered and dried to give the title compound as an off-white solid (0.059 g, 0.19 mmol, 45%). ¹H NMR (400 MHz, DMSO-d₆): δ 1.16-1.24 (m, 3H), 1.29-1.38 (m, 2H), 1.58-1.61 (m, 1H), 1.71-1.74 (m, 2H), 1.92-1.95 (m, 2H), 3.70-3.77 (m, 1H), 5.47 (br s, 1H), 6.33 (d, J=8.0 Hz, 1H), 6.69 (s, 1H), 6.71 (dd, J=1.6, 5.6 Hz, 1H), 7.30 (d, J=8.8 Hz, 1H), 7.50 (dd, J=1.6, 8.8 Hz, 1H), 7.97 (d, J=5.6 Hz, 1H), 8.04 (s, 1H), 11.50 (br s, 1H).

### Example 19

### 4-Bromo-N-(trans-4-methylcyclohexyl)pyridin-2-amine

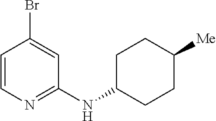

4-Bromo-2-fluoropyridine (0.1 mL, 0.98 mmol), triethylamine (0.50 mL, 3.59 mmol), trans-4-methylcyclohexanamine (0.4 mL, 2.98 mmol) in 1,4-dioxane (2.5 mL) was heated in the microwave at 160° C. for 30 min. Once cooled to room temperature the reaction mixture was concentrated under reduced pressure. Purification by column chromatography (100% Hexane—75/25 Hexane/EtOAc—100% EtOAc) afforded the title compound as an off white solid (0.22 g, 0.82 mmol, 83.8%) ¹H NMR (DMSO-d₆): δ 0.87 (d, J=6.4 Hz, 3H), 1.01 (2H, m), 1.13 (2H, m), 1.34 (1H, m), 1.68 (d, J=12.4 Hz, 2H), 1.91 (d, J=10.0 Hz, 2H), 3.58 (1H, m), 6.61 (3H, m), 7.82 (d, J=5.6 Hz, 1H). HRMS: For C₁₂H₁₈N₂Br requires 269.0648, found 269.0648.

### 5-{2-[(Trans-4-methylcyclohexyl)amino]pyridin-4-yl}-1H-indazol-3-amine

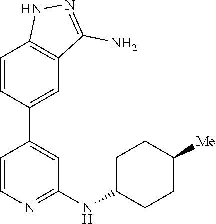

A suspension of 4-bromo-N-(trans-4-methylcyclohexyl)pyridin-2-amine (0.118 g, 0.44 mmol), 2-fluoro-5-(4,4,5,5-tetramethyl-1,3,2-dioxaborolan-2-yl)benzonitrile (0.169 g, 0.68 mmol), [1,1′-bis(di-tert-butylphosphino)ferrocene]dichloropalladium(II) catalyst (0.026 g, 0.04 mmol) in IPA/H₂O (3:1.5 mL) was degassed for 5 min using a steady stream of nitrogen. t-Butylamine (0.25 mL, 2.38 mmol) was added and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.3 mL, 6.00 mmol) was added prior to being reacted in the microwave at 165° C. for 30 min. Once cooled to room temperature the reaction mixture was concentrated under reduced pressure. Purification by column chromatography (100% EtOAc—10% MeOH/EtOAc) followed by trituration with Et₂O and filtration afforded the desired compound as an off white solid (0.11 g, 0.34 mmol, 78%)¹H NMR (DMSO-d₆): δ 0.90 (d, J=6.8 Hz, 3H), 1.05 (2H, m), 1.18 (2H, m), 1.36 (1H, m), 5.46 (2H, br s), 6.30 (d, J=8.0 Hz, 1H), 6.67 (1H, s), 6.71 (dd, J=5.2, 1.2 Hz, 1H), 7.30 (d, J=8.4 Hz, 1H), 7.50 (dd, J=1.6, 8.8 Hz, 1H), 7.97 (d, J=5.6 Hz, 1H), 8.04 (1H, s), 11.50 (1H, br s). HRMS: For C₁₉H₂₄N₅ requires 322.2024, found 322.2026.

### Example 20

### 2-((4-(3-Amino-1H-indazol-5-yl)pyridine-2-yl)amino)ethan-1-ol

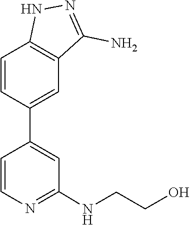 [SMILES: Nc1n[nH]c2ccc(-c3ccnc(NCCO)c3)cc12]

A suspension of 2-((4-bromopyridin-2-yl)amino)ethanol (0.117 g, 0.54 mmol), 3-cyano-4-fluorophenylboronic acid (0.218 g, 0.88 mmol), [1,1′-bis(di-tert-butylphosphino)ferrocene]dichloropalladium(II) catalyst (0.032 g, 0.049 mmol) in IPA/H₂O (3:1.5 mL) was degassed with nitrogen. t-Butylamine (0.30 mL, 2.85 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.3 mL, 6.00 mmol) and the mixture irradiated at 165° C. for 30 min. The reaction mixture was then concentrated under reduced pressure and purified using column chromatography (100% EtOAc—20% MeOH/EtOAc). The resulting solid was triturated with Et₂O, filtered and dried to give the title compound as an off-white solid (0.128 g, 0.48 mmol, 88%). ¹H NMR (400 MHz, DMSO-d₆): δ 3.41 (q, J=10.0 Hz, 2H), 3.59 (t, J=5.6, Hz, 2H), 5.60 (br s, 2H), 6.94 (t, J=3.8 Hz, 2H), 7.21 (s, 1H), 7.33 (d, J=8.8 Hz, 1H), 7.58 (dd, 1H, J=1.6, 8.8 Hz), 7.98 (d, 1H, J=5.6 Hz), 8.16 (d, 1H, J=1.2 Hz), 11.59 (br s, 1H). HRMS: For C₁₄H₁₆ON₅ requires 270.1349 found 270.1346.

### Example 21

### 3-((4-(3-Amino-1H-indazol-5-yl)pyridine-2-yl)amino)propan-1-ol

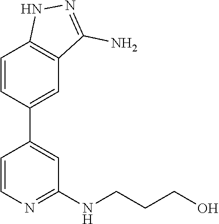 [SMILES: Nc1n[nH]c2ccc(-c3ccnc(NCCCO)c3)cc12]

A suspension of 3-((-4-bromopyridin-2-yl)amino)propan-1-ol (0.116 g, 0.72 mmol), 3-cyano-4-fluorophenylboronic acid (0.246 g, 1.00 mmol), [1,1′-bis(di-tert-butylphosphino)ferrocene]dichloropalladium(II) catalyst (0.037 g, 0.057 mmol) in IPA/H₂O (3:1.5 mL) was degassed with nitrogen. t-Butylamine (0.38 mL, 3.62 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.25 mL, 5.0 mmol) and the mixture irradiated at 165° C. for 30 min. The reaction mixture was then concentrated under reduced pressure and purified using column chromatography (100% EtOAc—20% MeOH/EtOAc). The resulting solid was triturated with Et₂O, filtered and dried to give the title compound as an off-white solid (0.0676 g, 0.239 mmol, 47.3%). ¹H NMR (400 MHz, DMSO-d₆): δ 1.79 (qt, 2H, J=6.4 Hz), 3.45 (q, 2H, J=6.4 Hz), 3.55 (t, 2H, J=6.0 Hz), 7.18 (d, 1H, J=6.8 Hz), 7.24 (s, 1H), 7.39 (d, 1H, J=8.8 Hz), 7.67 (d, 1H, J=8.4 Hz), 7.98 (d, 1H, J=6.8 Hz), 8.33 (s, 1H), 8.51 (br s, 1H), 11.80 (br s, 1H). HRMS: For C₁₅H₁₈ON₅ requires 284.1506 found 284.1504.

### Example 22

### 4-(4-(3-Amino-1H-indazol-5-yl)pyridin-2-ylamino)butan-1-ol

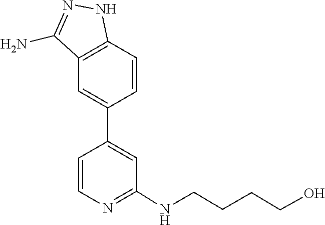 [SMILES: Nc1n[nH]c2ccc(-c3ccnc(NCCCCO)c3)cc12]

A suspension of 4-((3-bromopyridin-2-yl)amino)butan-1-ol (0.093 g, 0.38 mmol), 3-cyano-4-fluorophenylboronic acid pinacol ester (0.187 g, 0.76 mmol), [1,1′-bis(di-tert-butylphosphino)ferrocene]dichloropalladium(II) catalyst (0.040 g, 0.061 mmol) in IPA/H₂O (3:1.5 mL) was degassed with nitrogen. t-Butylamine (0.4 mL, 3.8 mmol) was added and the mixture was heated in the microwave at 160° C. for 45 min. The reaction mixture was concentrated and taken up in EtOH (4 mL) and degassed with nitrogen. Hydrazine hydrate (0.55 mL, 11.38 mmol) was added and the mixture was heated in the microwave at 165° C. for 30 min. The reaction mixture was concentrated and purified by flash column chromatography (silica gel with EtOAc 50% in hexane—MeOH 10% in EtOAc) to afford the title compound as a pale solid (0.070 g, 0.24 mmol, 62%) ¹H NMR (400 MHz, DMSO-d₆): 1.57 (m, 4H), 3.28 (q, J=6.8 Hz, 2H), 3.44 (q, J=6.4 Hz, 2H), 4.39 (t, J=5.2 Hz, 1H), 5.45 (br s, 2H), 6.45 (t, J=5.2 Hz, 1H), 6.70 (s, 1H), 6.76 (dd, J=1.6, 5.6 Hz, 1H), 7.31 (d, J=8.4 Hz, 1H), 7.53 (dd, J=1.6, 8.8 Hz, 1H), 7.99 (d, J=5.6 Hz, 1H), 8.07 (s, 1H), 11.49 (br s, 1H).

### Example 23

### 5-((4-(3-amino-1H-indazol-5-ylpyridin-2-yl)amino)pentan-1-ol

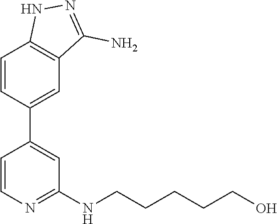 [SMILES: Nc1n[nH]c2ccc(-c3ccnc(NCCCCCO)c3)cc12]

A suspension of 5-[(4-bromopyridin-2-yl)amino]pentan-1-ol (0.150 g, 0.58 mmol), 2-fluoro-5-(4,4,5,5-tetramethyl-1,3,2-dioxaborolan-2-yl)benzonitrile (0.240 g, 0.96 mmol), [1,1′-bis(di-tert-butylphosphino)ferrocene]dichloropalladium(II) catalyst (0.047 g, 0.072 mmol) in IPA/H₂O (3:1.5 mL) was degassed for 5 min using a steady stream of nitrogen. t-Butylamine (0.3 mL, 2.9 mmol) was added and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.3 mL, 5.99 mmol) was added prior to being reacted in the microwave at 165° C. for 30 min. Once cooled to room temperature the reaction mixture was concentrated under reduced pressure. Purification by column chromatography (100% EtOAc—15% MeOH/EtOAc) followed by recrytallisation from MeOH (15 mL) and hexane (150 mL) and filtration afforded the desired compound as a yellow solid (0.099 g, 0.32 mmol, 55%) ¹H NMR (DMSO-d₆): δ 1.37-1.40 (2H, m), 1.41-1.5 (2H, m), 1.52-1.59 (2H, m), 3.26 (q, J=6.5 Hz, 2H), 3.40 (q, J=6.0 Hz, 2H), 4.37 (t, J=5.2 Hz, 1H), 5.48 (2H, br s), 6.55 (1H, br s), 6.77 (d, J=5.2 Hz, 1H), 7.30 (d, J=8.8 Hz, 1H), 7.53 (dd, J=8.8, 1.6 Hz, 1H), 7.98 (d, J=5.6 Hz, 1H), 8.07 (1H, s), 11.52 (1H, br s). HRMS: For C₁₇H₂₂ON₅ requires 312.1813, found 312.1819.

### Example 24

### 4-Bromo-N-(trans-4-hydroxycyclohexyl)pyridin-2-amine

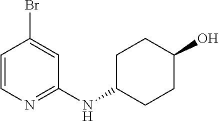

4-Bromo-2-fluoropyridine (0.5 ml, 4.52 mmol), (1r,4r)-4-aminocyclohexanol (1.200 g, 10.60 mmol) and triethylamine (0.7 ml, 5.02 mmol) were placed in a round bottom flask containing n-butanol (15 ml) and the solution was then heated for 16 hrs. The solvent was then removed under reduced pressure and the residue was dissolved in EtOAc (50 ml) and extracted with water (2×50 ml). The organic layer is then dried (Mg₂SO₄) and the sovent removed under reduced pressure. The residue was then purified by flash chromatography using hexane—hexane:ethylacetate (2:1) to give the desired product. (0.879 g, 3.2 mmol, 72.3%), δ_{H} (d₆-DMSO): 1.19 (4H, m), 1.82 (4H, m), 3.40 (1H, m), 3.57 (1H, m), 4.51 (d, J=4.0 Hz, 1H), 6.58 (2H, m), 6.62 (d, J=1.6 Hz, 1H), 7.82 (d, J=5.5 Hz, 1H), 9.32 (t, J=5.1 Hz, 1H). δ_{C} (d₆-DMSO): 30.8, 34.4, 49.0, 68.8, 110.7, 114.2, 131.9, 149.5, 159.7, LRMS: found: 273.00, 275.00 (M+1), calculated C₁₁H₁₅BrN₂O: 270.03.5-{2-[(Trans-4-hydroxycyclohexyl)amino]pyridin-4-yl}-1H-indazol-3-amine

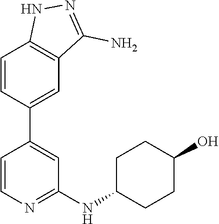 [SMILES: Nc1n[nH]c2ccc(-c3ccnc(N[C@H]4CC[C@H](O)CC4)c3)cc12]

4-Bromo-N-(trans-4-hydroxycyclohexyl)pyridin-2-amine (0.143 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.029 g, 0.09 mmol, 16.9%) δ_{H} (d₆-DMSO): 1.32 (4H, m), 1.88 (4H, m), 3.47 (1H, m), 3.64 (1H, m), 7.15 (dd, J=6.9, 1.7 Hz, 1H), 7.21 (d, J=1.0 Hz, 1H), 7.40 (d, J=8.8 Hz, 1H), 7.67 (d, J=8.65 Hz, 1H), 7.96 (d, J=6.9 Hz, 1H), 8.30 (1H, s), 8.55 (1H, s). HRMS: C₁₈H₂₁N₅O requires 323.1746, found 324.1819 (M+H).

### Example 25

### 5-(2-((2-Methoxyethyl)amino)pyridine-4-yl)-1H-indazol-3-amine

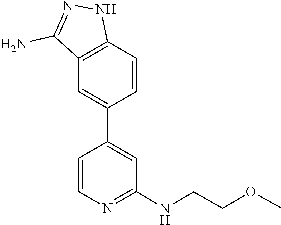 [SMILES: COCCNc1cc(-c2ccc3[nH]nc(N)c3c2)ccn1]

A suspension of 4-bromo-N-(2-methoxyethyl)pyridin-2-amine (0.040 g, 0.17 mmol), 3-cyano-4-fluorophenylboronic acid (0.075 g, 0.30 mmol), [1,1′-bis(di-tert-butylphosphino)ferrocene]dichloropalladium(II) catalyst (0.015 g, 0.02 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 5.0 mmol) and the mixture irradiated at 165° C. for 30 min. The reaction mixture was then concentrated under reduced pressure and purified using column chromatography (100% EtOAc—20% Methanol/EtOAc). The resulting solid was recrystalised from methanol and hexane, filtered and dried to give the title compound as an off-white solid (0.037 g, 0.13 mmol, 49%). ¹H NMR (400 MHz, DMSO-d₆): δ 3.28 (s, 3H), 3.48 (br s, 2H), 5.47 (br s, 2H), 6.55 (br s, 1H), 6.76 (s, 1H), 6.78 (d, J=5.6 Hz, 1H), 7.31 (d, J=8.8 Hz, 1H), 7.51 (dd, J=1.2, 8.4 Hz, 1H), 7.99 (d, J=5.6 Hz, 1H), 8.06 (s, 1H), 11.51 (br s, 1H).

### Example 26

### 5-(2-((3-Methoxypropyl)amino)pyridine-4-yl)-1H-indazol-3-amine

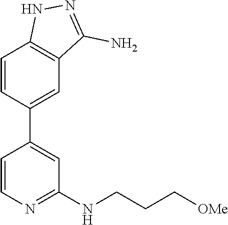 [SMILES: COCCCNc1cc(-c2ccc3[nH]nc(N)c3c2)ccn1]

A suspension of 4-bromo-N-(3-methoxypropyl)pyridine-2-amine (0.030 g, 0.12 mmol), 3-cyano-4-fluorophenylboronic acid (0.056 g, 0.23 mmol), [1,1′-bis(di-tert-butylphosphino)ferrocene]dichloropalladium(II) catalyst (0.013 g, 0.02 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.20 mL, 5.0 mmol) and the mixture irradiated at 165° C. for 30 min. The reaction mixture was then concentrated under reduced pressure and purified using column chromatography (100% EtOAc—20% Methanol/EtOAc). The resulting solid was recrystalised from methanol and EtOAc, filtered and dried under reduced pressure to give the title compound as an off-white solid (0.022 g, 0.07 mmol, 60%). ¹H NMR (400 MHz, DMSO-d₆): δ 1.79 (qt, J=6.6 Hz, 2H), 3.25 (s, 3H), 3.42 (t, J=6.3 Hz, 2H), 5.45 (br s, 2H), 6.48 (t, J=5.3 Hz, 1H), 6.69 (s, 1H), 6.77 (dd, J=1.4, 5.4 Hz, 1H), 7.30 (d, J=8.6 Hz, 1H), 7.52 (dd, J=1.6, 8.7 Hz, 1H), 7.99 (d, J=5.4 Hz, 1H), 8.06 (s, 1H), 11.49 (br s, 1H). HRMS: For C₁₆H₁₉ON₅ requires 298.1662 found 298.1661.

### Example 27

### 4-Bromo-N-[3-(propan-2yloxy)propyl]pyridine-2-amine

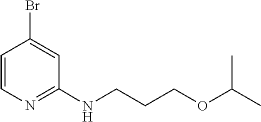

4-Bromo-2-fluoropyridine (0.1 mL, 0.98 mmol), triethylamine (0.55 mL, 3.95 mmol), 3-(propan-2-yloxy)propan-1-amine (0.68 mL, 4.90 mmol) in 1,4-dioxane (2.5 mL) was heated in the microwave at 160° C. for 30 min. Once cooled to room temperature the reaction mixture was concentrated under reduced pressure. Purification by column chromatography (100% Hexane—75/25 Hexane/EtOAc) afforded the title compound as an off white solid (0.21 g, 0.77 mmol, 79%). ¹H NMR (DMSO-d₆): δ 1.07 (d, J=6.0 Hz, 6H), 1.69 (2H, m), 3.24 (q, J=6.5 Hz, 2H), 3.41 (t, J=2.8 Hz, 2H), 3.49 (2H, m), 6.63 (dd, J=5.2, 1.6 Hz, 1H), 6.66 (d, J=1.6 Hz, 1H), 6.73 (t, J=10.8 Hz, 1H), 7.83 (d, J=5.6 Hz, 1H). LRMS: found: 273.00, 274.93 (M+1), calculated C₁₁H₁₇BrN₂O: 272.05.

### 5-(2-((3-Isopropoxypropyl)amino)pyridin-4-yl)-1H-indazol-3-amine

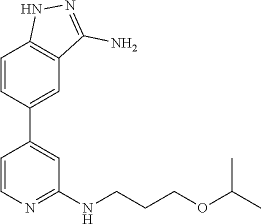 [SMILES: CC(C)OCCCNc1cc(-c2ccc3[nH]nc(N)c3c2)ccn1]

A suspension of 4-bromo-N-[3-(propan-2yloxy)propyl]pyridine-2-amine (0.196 g, 0.72 mmol), 2-fluoro-5-(4,4,5,5-tetramethyl-1,3,2-dioxaborolan-2-yl)benzonitrile (0.280 g, 1.14 mmol), [1,1′-bis(di-tert-butylphosphino)ferrocene]dichloropalladium(II) catalyst (0.050 g, 0.08 mmol) in IPA/H₂O (3:1.5 mL) was degassed for 5 min using a steady stream of nitrogen. t-Butylamine (0.38 mL, 3.6 mmol) was added and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.38 mL, 7.60 mmol) was added prior to being reacted in the microwave at 165° C. for 30 min. Once cooled to room temperature the reaction mixture was concentrated under reduced pressure. Purification by column chromatography (100% Hexane—50/50 Hexane/EtOAc—100% EtOAc—10% MeOH/EtOAc) followed by recrytallisation from MeOH (15 mL) and hexane (150 mL) and filtration afforded the desired compound as a yellow solid (0.114 g, 0.35 mmol, 48.7%). ¹H NMR (DMSO-d₆): δ 1.09 (d, J=6.0 Hz, 6H), 1.77 (qt, J=6.5 Hz, 2H), 3.46 (t, J=12.4 Hz, 2H), 3.53 (qt, J=6.1 Hz, 1H), 5.50 (1H, br s), 6.81 (1H, s), 6.89 (d, J=3.2 Hz, 1H), 7.32 (d, J=8.4 Hz, 1H), 7.56 (d, J=8.8 Hz, 1H), 7.99 (d, J=5.6 Hz, 1H), 8.12 (1H, s). 11.56 (1H, br s). HRMS: For C₁₈H₂₄ON₅ requires 326.1977, found 326.1975.

### Example 28

### 3-((4-Chloropyrimidin-2-yl)amino)propan-1-ol

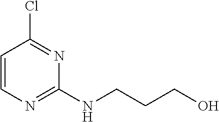 [SMILES: OCCCNc1nccc(Cl)n1]

2,4-Dichloropyrimidine (2 g, 13.42 mmol) and triethylamine (1.5 ml, 21.21 mmol) were placed in a round bottom flask with dichloromethane (30 ml). 3-aminopropan-1-ol (2.5 ml, 33.50 mmol) was then added dropwise and the solution allowed to stir overnight at room temperature. Water (20 ml) was then added and the organic layer dried over sodium sulfate, filtered, and the solvent removed under reduced pressure to give the crude product which was purified by flash chromatography (100% Heaxane—50/50 hexane/EtOAc) to yield the desired products (1) and (II). (0.562 g, 3.00 mmol, 22%), δ_{H} (d₆-DMSO): 1.65 (q, J=6.5 Hz, 2H), 3.28 (2H, m), 3.44 (t, J=6.5 Hz, 2H), 4.43 (1H, s), 6.61 (d, J=5.0 Hz, 1H), 7.58 (t, J=5.5 Hz, 1H), 8.21 (1H, s). LRMS: found: 188.00 (M+1), calculated C₇H₁₀ClN₃O: 187.05

### 3-((4-(3-Amino-1H-indazol-5-yl)pyrimidin-2-yl)amino)propan-1-ol

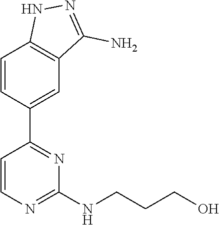 [SMILES: Nc1n[nH]c2ccc(-c3ccnc(NCCCO)n3)cc12]

3-((4-Chloropyrimidin-2-yl)amino)propan-1-ol (0.103 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (32 mg, 0.1 mmol, 20.8%) δ_{H} (d₆-DMSO): 1.77 (t, J=6.1 Hz, 2H), 3.53 (4H, m), 4.48 (1H, s), 7.27 (1H, s), 7.39 (d, J=6.2 Hz, 1H), 8.06 (1H, s), 8.16 (1H, m), 8.35 (d, J=6.2 Hz, 1H), 8.73 (1H, s). HRMS: C₁₄H₁₆N₆O requires 284.1386, found 285.1458 (M+H)⁺

### Example 29

### 3-((4-(3-Amino-1H-indazol-5-yl)pyridin-2-yl)(methyl)amino)propan-1-ol

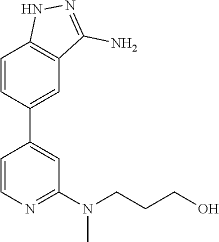 [SMILES: CN(CCCO)c1cc(-c2ccc3[nH]nc(N)c3c2)ccn1]

A suspension of 3-((4-bromopyridin-2-yl)(methyl)amino)propan-1-ol (0.027 g, 0.11 mmol), 3-cyano-4-fluorophenylboronic acid pinacol ester (0.054 g, 0.22 mmol), [1,1′-bis(di-tert-butylphosphino)ferrocene]dichloropalladium(II) catalyst (0.014 g, 0.02 mmol) in IPA/H₂O (3:1.5 mL) was degassed with nitrogen. t-Butylamine (0.12 mL, 1.10 mmol) was added and the mixture was heated in the microwave at 160° C. for 45 min. The reaction mixture was concentrated and taken up in EtOH (4 mL) and degassed with nitrogen. Hydrazine hydrate (0.16 mL, 3.30 mmol) was added and the mixture was heated in the microwave at 165° C. for 30 min. The reaction mixture was concentrated and purified by flash column chromatography (silica gel with EtOAc 50% in hexane—MeOH 10% in EtOAc) to afford the title compound as an off-white solid (0.008 g. 0.03 mmol, 24%). ¹H NMR—(500 MHz, DMSO-d₆): 1.72 (m, 2H), 3.06 (m, 3H), 3.44 (q, J=6.0 Hz, 2H), 3.62 (t, J=7.1 Hz, 2H), 6.81 (s, 1H), 6.83 (dd, J=1.5, 5.5 Hz, 1H), 7.29 (d, J=9.0 Hz, 1H), 7.61 (dd, J=1.5, 8.5 Hz, 1H), 8.08 (d, J=5.0 Hz, 1H), 8.12 (s, 1H), 11.49 (br s, 1H). HRMS: C₁₆H₁₉ON₅ requires 297.1590, found 298.1662 (M+H)+

### Example 30

### 5-(2-((2-Morpholinoethyl)amino)pyridine-4-yl)-1H-indazol-3-amine

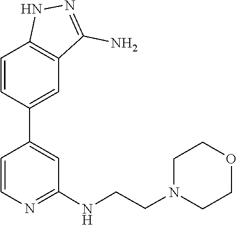 [SMILES: Nc1n[nH]c2ccc(-c3ccnc(NCCN4CCOCC4)c3)cc12]

A suspension of 4-bromo-N-(2-morpholinoethyl)pyridine-2-amine (0.100 g, 0.35 mmol), 3-cyano-4-fluorophenylboronic acid (0.151 g, 0.61 mmol), [1,1′-bis(di-tert-butylphosphino)ferrocene]dichloropalladium(II) catalyst (0.021 g, 0.032 mmol) in IPA/H₂O (3:1.5 mL) was degassed with nitrogen. t-Butylamine (0.20 mL, 1.90 mmol) was then added and degassed using nitrogen. The reaction was placed in a microwave and irradiated at 160° C. for 40 min. The reaction was diluted with EtOAc and concentrated under reduced pressure. EtOH (4.5 mL) was added followed by hydrazine hydrate (0.3 mL, 6.0 mmol) and the mixture irradiated at 165° C. for 30 min. The reaction mixture was then concentrated under reduced pressure and purified using column chromatography (100% EtOAc—20% MeOH/EtOAc). The resulting solid was triturated with Et₂O, filtered and dried to give the title compound as an off-white solid (0.0379 g, 0.11 mmol, 32.1%). ¹H NMR (400 MHz, DMSO-d₆): δ 2.40-2.44 (m, 4H), 3.41 (q, J=6.4, 12.4 Hz, 2H), 3.59 (m, 4H), 5.48 (br s, 2H), 6.35 (t, 1H), 6.74 (s, 1H), 6.78 (dd, J=1.2, 5.2 Hz, 1H), 7.30 (d, J=8.8 Hz, 1H), 7.54 (dd, J=1.6, 8.8 Hz, 1H), 7.99 (d, J=5.6 Hz, 1H), 8.08 (s, 1H), 11.52 (br s, 1H). HRMS: For C₁₈H₂₃ON₆ requires 339.1928 found 339.1927.

### Example 31

### 5-(2-((2-(Piperidin-1-yl)ethyl)amino)pyridine-4-yl)-1H-indazol-3-amine

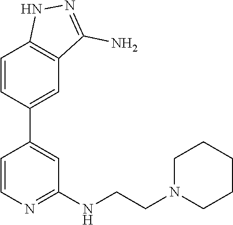 [SMILES: Nc1n[nH]c2ccc(-c3ccnc(NCCN4CCCCC4)c3)cc12]

A suspension of 4-bromo-N-(2-(piperidin-1-yl)ethyl)pyridine-2-amine (0.110 g, 0.39 mmol), 3-cyano-4-fluorophenylboronic acid (0.155 g, 0.63 mmol), [1,1′-bis(di-tert-butylphosphino)ferrocene]dichloropalladium(II) catalyst (0.030 g, 0.05 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.0 mmol) and the mixture irradiated at 165° C. for 30 min. The reaction mixture was then concentrated under reduced pressure and purified using column chromatography (100% EtOAc—20% Methanol/EtOAc). The resulting solid was recrytalised from methanol and hexane, filtered and dried to give the title compound as an off-white solid (0.011 g, 0.003 mmol, 8.7%). ¹H NMR (400 MHz, DMSO-d₆): δ 1.39-1.40 (m, 2H), 150-1.53 (m, 4H), 2.39-2.47 (m, 4H), 3.38 (q, J=6.2 Hz, 2H), 5.47 (br s, 2H), 6.29 (t, J=4.8 Hz, 1H), 6.73 (s, 1H), 6.77 (dd, J=5.2, 11.2 Hz, 1H), 7.30 (d, J=8.8 Hz, 1H), 7.54 (d, J=8.4 Hz, 1H), 7.99 (d, J=5.2 Hz, 1H), 8.08 (s, 1H), 11.51 (br s, 1H). HRMS: For C₁₉H₂₄N₆ requires 337.2135 found 337.2133.

### Example 32

### tert-Butyl 3-(3-(3-amino-1H-indazol-5-yl)phenylamino)propyl methyl)carbamate

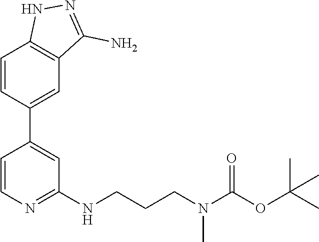 [SMILES: CN(CCCNc1cc(-c2ccc3[nH]nc(N)c3c2)ccn1)C(=O)OC(C)(C)C]

A suspension of tert-butyl 3-(3-bromophenylamino)propyl(methyl)carbamate (0.105 g, 0.31 mmol), 3-cyano-4-fluorophenylboronic acid pinacol ester (0.150 g, 0.61 mmol), [1,1′-bis(di-tert-butylphosphino)ferrocene]dichloropalladium(II) catalyst (0.040 g, 0.06 mmol) in IPA/H₂O (3:1.5 mL) was degassed with nitrogen. t-Butylamine (0.32 mL, 3.05 mmol) was added and the mixture was heated in the microwave at 160° C. for 45 min. The reaction mixture was concentrated and taken up in EtOH (4 mL) and degassed with nitrogen. Hydrazine hydrate (0.44 mL, 9.15 mmol) was added and the mixture was heated in the microwave at 165° C. for 30 min. The reaction mixture was concentrated and purified by flash column chromatography (silica gel with EtOAc 50% in hexane—MeOH 10% in EtOAc) to afford the title compound as an off-white solid (0.032 g, 0.08 mmol, 26%). ¹H NMR (400 MHz, DMSO-d₆): 1.37 (br s, 9H), 1.75 (m, 2H), 2.80 (s, 3H), 3.26 (m, 4H), 5.45 (br s, 2H), 6.45 (br s, 1H), 6.69 (s, 1H), 6.78 (dd, J=1.6, 5.6 Hz, 1H), 7.31 (d, J=8.4 Hz, 1H), 7.53 (d, J=8.8 Hz, 1H), 8.0 (d, J=5.2 Hz, 1H), 8.07 (s, 1H), 11.50 (br s, 1H). HRMS: C₂₁H₂₈O₂N₅ requires 396.2274, found 397.2347 (M+H)

### N¹-(4-(3-Amino-1H-indazol-5-yl)pyridin-2-yl)-N³-methylpropane-1,3-diamine

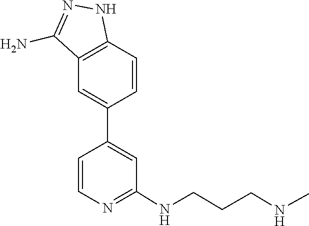 [SMILES: CNCCCNc1cc(-c2ccc3[nH]nc(N)c3c2)ccn1]

To a mixture of tert-butyl 3-(3-(3-amino-1H-indazol-5-yl)phenylamino)propyl(methyl)carbamate (0.02 g, 0.05 mmol) in anhydrous dichloromethane (0.1 mL) was added TFA (0.1 mL, 1.31 mmol) at 0° C. The reaction was stirred for 1 h at 0° C. and then 1 h at room temperature. 2M NaOH (0.7 mL) was added and the stirring continued for a further 10 min. The reaction was extracted and washed with dichloromethane (1 mL×2 ¹H NMR (400 MHz, DMSO-d₆): 1.91 (tt, J=7.2 Hz, 2H), 2.60 (t, J=5.4 Hz, 3H), 3.01 (m, 2H), 3.47 (m, 2H), 7.19 (br s, 2H), 7.39 (d, J=8.8 Hz), 7.70 (d, J=9.5 Hz, 1H), 8.05 (d, J=7.2 Hz, 1H), 8.32 (s, 1H), 8.48 br s, 1H), 11.80 (br s, 1H).

### Section 5—Compounds of the Formula

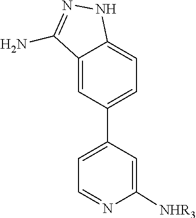

### Example 33

### N-[4-(3-Amino-1H-indazol-5-yl)pyridin-2-yl]cyclopropanecarboxamide

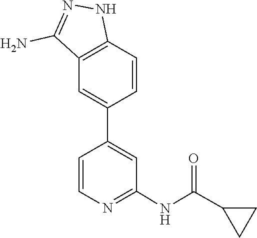 [SMILES: Nc1n[nH]c2ccc(-c3ccnc(NC(=O)C4CC4)c3)cc12]

A suspension of N-(4-chloropyridin-2-yl)cyclopropanecarboxamide (0.133 g, 0.678 mmol), 3-cyano-4-fluorophenylboronic acid (0.205 g, 0.791 mmol), [1,1′-bis(di-tert-butylphosphino)ferrocene]dichloropalladium(II) catalyst (0.043 g, 0.066 mmol) in IPA/H₂O (3:1.5 mL) was degassed with nitrogen. t-Butylamine (0.60 mL, 5.71 mmol) was then added and degassed using nitrogen. The reaction was placed in a microwave and irradiated at 160° C. for 40 min. The reaction was diluted with EtOAc, concentrated under reduced pressure, To this residue was added ethanol (5 mL) and hydrazine hydrate (0.25 mL, 5.0 mmol) and the reaction mixture was placed in a microwave and irradiated at 165° C. for 30 min. Purification was carried out using HPLC as described in the general experimental section. Peak collected at 9 mins. (0.01229 g, 0.04 mmol, 6.2%). ¹H NMR (400 MHz, DMSO-d₆): δ 0.83-0.86 (m, 4H), 2.04-2.07 (m, 1H), 7.36-7.40 (m, 2H), 7.63 (d, J=8.4 Hz, 1H), 8.19 (s, 1H), 8.34 (d, J=5.2 Hz, 1H), 8.43 (s, 1H), 10.84 (s, 1H), 11.72 (br s, 1H). LRMS: found: 294.13 (M+1), calculated C₁₆H₁₅N₅O: 293.13

### Example 34

### N-(4-(3-Amino-1H-indazol-5-yl)pyridin-2-yl)benzamide

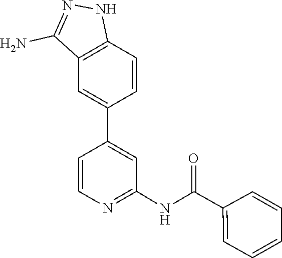 [SMILES: Nc1n[nH]c2ccc(-c3ccnc(NC(=O)c4ccccc4)c3)cc12]

To a suspension of N-(4-bromopyridin-2-yl)benzamide (0.097 g, 0.35 mmol), 5-(4,4,5,5-tetramethyl-1,3,2-dioxaborolan-2-yl)-1H-indazol-3-amine (0.136 g, 0.525 mmol) and tetrakis(triphenylphosphine)palladium(0) catalyst (0.02 g, 0.0175 mmol) in EtOH/water (1:1; 1.5 mL, degassed under nitrogen) was added K₃PO₄ (1M, 0.88 mL) and the reaction mixture was heated to 80° C. for 24 h. The reaction mixture was cooled to room temperature, filtered and the resulting solid was washed with water (10 mL) and Et₂O (8 mL) and then dried. Purification by column chromatography (100% Hexane—2/1 Hexane/EtOAc—100% EtOAc) and trituration with MeOH and hexane afforded the title compound as a grey solid (0.05 g, 43%). ¹H NMR (500 MHz, DMSO) δ 11.60 (s, 1H), 10.81 (s, 1H), 8.57 (d, J=1.7 Hz, 1H), 8.43 (d, J=5.3 Hz, 1H), 8.24 (d, J=1.7 Hz, 1H), 8.11-8.06 (m, 2H), 7.70-7.62 (m, 1H), 7.61 (s, 1H), 7.55 (t, J=7.6 Hz, 2H), 7.48 (dd, J=5.2, 1.8 Hz, 1H), 7.39 (d, J=8.7 Hz, 1H), 5.56 (s, 2H).

LRMS: found: 330.13 (M+1), calculated C₁₅H₁₆N₅O: 330.14 (M+1)

### Example 35

### Ethyl (4-(3-amino-1H-indazol-5-yl)pyridin-2-yl)carbamate

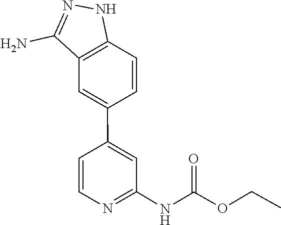 [SMILES: CCOC(=O)Nc1cc(-c2ccc3[nH]nc(N)c3c2)ccn1]

Ethyl (4-bromopyridin-2-yl)carbamate (0.437 g, 1.82 mmol) along with 5-(4,4,5,5-tetramethyl-1,3,2-dioxaborolan-2-yl)-1H-indazol-3-amine (0.722 g, 2.16 mmol), and potassium phosphate (945 mg in water (4 ml)) were placed in a 5 ml microwave vial to containing ethanol (10 ml). The solution was then degassed with N₂ for 10 min. [1,1′-bis(di-tert-butylphosphino)ferrocene]dichloropalladium(II) catalyst (118) (0.060 g, 0.10 mmol, 5 mol %) was then added and the vial sealed and heated to 90° C. for 16 hrs. The solution was then allowed to cool before removing the solvent under reduced pressure to give the crude product which was purified by HPLC to give the desired product. (569 mg, 1.70 mmol, 94%), δ_{H} (d₆-DMSO): 1.25 (t, J=7.0 Hz, 3H), 4.02 (q, J=7.0 Hz, 2H), 5.52 (s, 2H) 7.32 (m, 2H), 7.59 (dd, J=8.5 and 1.5 Hz, 1H), 8.15 (m, 2H), 8.27 (d, J=5.5 Hz, 1H), 10.08 (s, 1H), 11.56 (s, 1H).

LRMS: found: 298.10 (M+1), calculated C₁₅H₁₅N₅O₂: 297.12.

### Example 36

### 1-(4-Chloropyridin-2-yl)-3-ethylurea

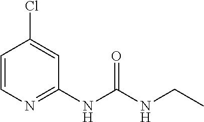

A 2-5 mL microwave vial was charged with 4-chloropyridin-2-amine (437 mg, 3.40 mg, 1 eq.) and chloroform (1.6 mL), and to the resulting suspension was added isocyanatoethane (483 mg, 538 μL, 6.80 mmol, 2 eq.). The reaction mixture was heated to 100° C. under microwave irradiation for 1 hour. The resulting light yellow solution was cooled to room temperature, diluted by dropwise addition of hexane (8 mL) and left overnight. The white precipitate was then filtered and washed with hexane (8 mL×3) to give the title compound (480 mg, 71%) as a white solid. ¹H NMR (DMSO-D6) δ: 1.07 (t, J=7.2 Hz, 3H), 3.21-3.12 (m, 2H), 7.04 (dd, J=5.5, 1.9 Hz, 1H), 7.59 (d, J=1.6 Hz, 1H), 7.62 (br s, 1H), 8.16 (d, J=5.5 Hz, 1H), 9.30 (s, 1H)

### 1-(4-(3-Amino-1H-indazol-5-yl)pyridine-2-yl)-3-ethylurea

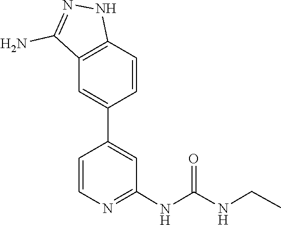 [SMILES: CCNC(=O)Nc1cc(-c2ccc3[nH]nc(N)c3c2)ccn1]

A 2-5 mL microwave vial was charged 1-(4-chloropyridin-2-yl)-3-ethylurea (200 mg, 1 mol, 1 eq), 5-(4,4,5,5-tetramethyl-1,3,2-dioxaborolan-2-yl)-1H-indazol-3-amine (259 mg, 1.2 mmol, 1.2 eq.), [1,1′-bis(di-tert-butylphosphino)ferrocene]dichloropalladium(II) catalyst (33 mg, 0.05 mmol, 5 mol %), sealed with an aluminium crimp cap fitted with a disposable PTFE/silicon septum, and purged with nitrogen. Ethanol (4 mL) was added and the mixture was stirred under nitrogen for some minutes before adding 1 M aqueous K₃PO₄ (2.0 mL, O₂-free). The mixture was heated to ca. 50° C., purged with nitrogen for 10 minutes and then left stirring at 60° C. for 24 hours. The reaction mixture was then diluted with EtOAc (200 mL), washed with water (40 ml×3), dried (MgSO₄) and purified by flash chromatography (Biotage SP4, 50 g SiO₄, 5% MeOH in EtOAc) to afford the title compound (53 mg, 18%) as an off-white solid ¹H NMR (DMSO-D6) δ: 1.11 (t, J=7.2 Hz, 3H), 3.25-3.17 (m, 2H), 5.53 (s, 2H), 7.20 (dd, J=5.4, 1.6 Hz, 1H), 7.34 (d, J=8.7 Hz, 1H), 7.54 (dd, J=8.7, 1.6 Hz, 1H), 7.67 (s, 1H), 8.12 (s, 1H), 8.15 (br s, 1H), 8.20 (d, J=5.4 Hz, 1H), 9.17 (s, 1H), 11.58 (s, 1H). HRMS: Calculated for C15H17ON6 (M+H⁺): 297.1458; Found: 297.1450

### Example 37

### 1-(4-(3-Amino-1H-indazol-5-yl)pyridin-2-yl)-3-propylurea

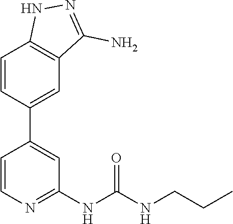 [SMILES: CCCNC(=O)Nc1cc(-c2ccc3[nH]nc(N)c3c2)ccn1]

1-(4-Bromopyridin-2-yl)-3-propylurea (0.136 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.052 g, 0.17 mmol, 32.8%) δ_{H} (d₆-DMSO): 0.91 (t, J=7.4 Hz, 3H), 1.50 (h, J=7.25 Hz, 2H), 3.16 (q, J=6.9 Hz, 2H), 7.36 (d, J=4.8 Hz, 1H), 7.43 (d, J=8.8 Hz, 1H), 7.67 (m, 2H), 7.93 (s, 1H), 8.26 (m, 2H). HRMS: C₁₆H₁₈N₆O requires 310.1542, found 311.1615 (M+H)⁺.

### Example 38

### 1-(4-(3-Amino-1H-indazol-5-yl)pyridin-2-yl)-3-isopentylurea

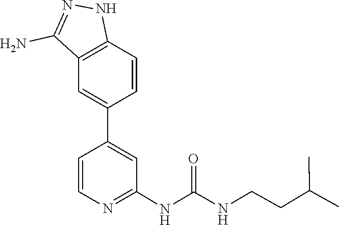 [SMILES: CC(C)CCNC(=O)Nc1cc(-c2ccc3[nH]nc(N)c3c2)ccn1]

A 0.5-2 mL microwave vial was charged with ethyl (4-(3-amino-1H-indazol-5-yl)pyridin-2-yl)carbamate (Example 35) (0.030 g, 0.10 mmol, 1 eq.), 3-methylbutan-1-amine (0.264 g, 352 μL, 3.03 mmol, 30 eq.) and dioxane (650 μL), was sealed with an aluminium crimp cap fitted with a disposable PTFE/silicon septum, and purged with nitrogen. The reaction mixture was heated to 180° C. under microwave irradiation for 30 minutes. The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound. (32 mg, 0.09 mmol, 94%) ¹H NMR (DMSO-D6) δ: 0.91 (d, J=6.6 Hz, 6H), 1.39 (dd, J=14.3, 7.1 Hz, 2H), 1.67-1.57 (m, 1H), 3.26-3.15 (m, 2H), 5.53 (s, 2H), 7.20 (d, J=5.5 Hz, 1H), 7.34 (d, J=8.7 Hz, 1H), 7.54 (d, J=8.6 Hz, 1H), 7.67 (s, 1H), 8.23-8.09 (m, 3H), 9.16 (s, 1H), 11.58 (s, 1H), HRMS: Calculated for C₁₈H₂₃ON₆(M+H⁺): 339.1928; Found: 339.1925

### Example 39

### 1-(4-(3-Amino-1H-indazol-5-yl)pyridin-2-yl)-3-cyclopentylurea

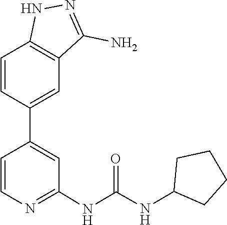 [SMILES: Nc1n[nH]c2ccc(-c3ccnc(NC(=O)NC4CCCC4)c3)cc12]

1-(4-Bromopyridin-2-yl)-3-cyclopentylurea (0.149 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.028 g, 0.08 mmol, 16%) δ_{H} (d₆-DMSO): 1.43 (m, 2H), 1.56 (m, 2H), 1.66 (m, 2H), 1.89 (m, 2H), 4.02 (h, J=6.5 Hz, 1H), 7.29 (s, 1H), 7.39 (d, J=9.0 Hz, 1H), 7.62 (d, J=8.8 Hz, 1H), 7.72 (s, 1H), 8.22 (m, 2H). HRMS: C₁₈H₂₀N₆O requires 336.1699, found 337.1771 (M+H)⁺.

### Example 40

1-(4-(3-Amino-1H-indazol-5-yl)pyridin-2-yl)-3-cyclohexylurea

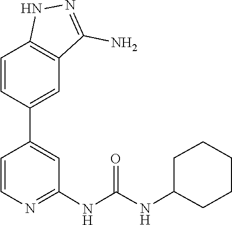 [SMILES: Nc1n[nH]c2ccc(-c3ccnc(NC(=O)NC4CCCCC4)c3)cc12]

1-(4-Bromopyridin-2-yl)-3-cyclopentylurea (0.157 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.043 g, 0.12 mmol, 22%) δ_{H} (d₆-DMSO): 1.18 (m, 6H), 1.67 (m, 2H), 1.86 (m, 2H), 3.58 (m, 1H), 7.32 (s, 1H), 7.42 (d, J=8.7 Hz, 1H), 7.62 (dd, J=8.8 and 1.4 Hz, 1H), 7.71 (s, 1H), 8.24 (m, 2H). HRMS: C₁₉H₂₂N₆O requires 350.1855, found 351.1928 (M+H)⁺.

### Example 41

### 1-(4-(3-Amino-1H-indazol-5-yl)pyridin-2-yl)-3-(2-hydroxyethyl)urea

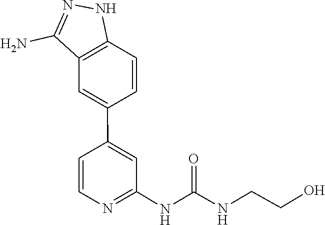 [SMILES: Nc1n[nH]c2ccc(-c3ccnc(NC(=O)NCCO)c3)cc12]

A 0.5-2 mL microwave vial was charged with ethyl (4-(3-amino-1H-indazol-5-yl)pyridin-2-yl)carbamate (Example 35) (0.030 g, 0.10 mmol, 1 eq.), 2-aminoethanol (0.025 g, 24.4 μL, 0.40 mmol, 4 eq.) and dioxane (650 μL), was sealed with an aluminium crimp cap fitted with a disposable PTFE/silicon septum, and purged with nitrogen. The reaction mixture was heated to 160° C. under microwave irradiation for 90 minutes. The reaction mixture was then concentrated in vacuo and the resulting crude was dissolved in water (3 mL). The mixture was stirred and sonicated for 30 minutes, then the solid was filtered, washed with water (2×3 mL) and dried to afford the title compound. (27 mg, 0.08 mmol, 84%) ¹H NMR (400 MHz, DMSO-D6) δ 3.25 (app q, J=5.6 Hz, 2H), 3.48 (m, 2H), 4.78 (t, J=5.1 Hz, 1H), 5.53 (s, 2H), 7.20 (dd, J=5.4, 1.5 Hz, 1H), 7.34 (d, J=8.7 Hz, 1H), 7.54 (dd, J=8.7, 1.5 Hz, 1H), 7.68 (s, 1H), 8.12 (s, 1H), 8.20 (d, J=5.4 Hz, 1H), 8.27 (br s, 1H), 9.24 (s, 1H), 11.58 (s, 1H). HRMS: Calculated for C₁₅H₁₇O₂N₆(M+H⁺): 313.1408; Found: 313.1400

### Example 42

### 1-(4-(3-Amino-1H-indazol-5-yl)pyridin-2-yl)-3-(3-hydroxypropyl)urea

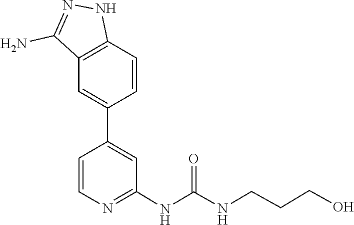 [SMILES: Nc1n[nH]c2ccc(-c3ccnc(NC(=O)NCCCO)c3)cc12]

A 0.5-2 mL microwave vial was charged with ethyl (4-(3-amino-1H-indazol-5-yl)pyridin-2-yl)carbamate (Example 35) (0.030 g, 0.10 mmol, 1 eq.), 3-aminopropan-1-ol (0.030 g, 30.9 μL, 0.40 mmol, 4 eq.) and dioxane (650 μL), was sealed with an aluminium crimp cap fitted with a disposable PTFE/silicon septum, and purged with nitrogen. The reaction mixture was heated to 180° C. under microwave irradiation for 40 minutes. The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound. (17 mg, 0.05 mmol, 52%) ¹H NMR (400 MHz, DMSO-D₆) δ 1.72-1.55 (m, 2H), 3.28-3.21 (m, 2H), 3.52-3.44 (m, 2H), 4.51 (t, J=5.1 Hz, 1H), 5.53 (s, 2H), 7.21 (d, J=5.5 Hz, 1H), 7.34 (d, J=8.8 Hz, 1H), 7.54 (d, J=8.8 Hz, 1H), 7.66 (s, 1H), 8.12 (s, 1H), 8.28-8.15 (m, 2H), 9.20 (s, 1H), 11.58 (s, 1H). HRMS: Calculated for C₁₆H₁₉O₂N₆(M+H⁺): 327.1564; Found: 327.1558

### Example 43

### 1-(4-(3-Amino-1H-indazol-5-yl)pyridin-2-yl)-3-(2-methoxyethyl)urea

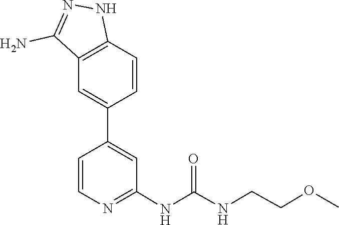 [SMILES: COCCNC(=O)Nc1cc(-c2ccc3[nH]nc(N)c3c2)ccn1]

A 0.5-2 mL microwave vial was charged with ethyl (4-(3-amino-1H-indazol-5-yl)pyridin-2-yl)carbamate (Example 35) (0.030 g, 0.10 mmol, 1 eq.), 2-methoxyethanamine (0.264 g, 352 μL, 3.03 mmol, 30 eq.) and dioxane (650 μL), was sealed with an aluminium crimp cap fitted with a disposable PTFE/silicon septum, and purged with nitrogen. The reaction mixture was heated to 180° C. under microwave irradiation for 30 minutes. The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound. (0.029 g, 0.09 mmol, 88%), ¹H NMR (DMSO-D₆) 5: 3.29 (s, 3H), 3.38-3.34 (m, 2H), 3.45-3.40 (m, 2H), 5.53 (s, 2H), 7.21 (d, J=5.3 Hz, 1H), 7.34 (d, J=8.7 Hz, 1H), 7.54 (d, J=8.8 Hz, 1H), 7.70 (s, 1H), 8.12 (s, 1H), 8.20 (d, J=5.4 Hz, 1H), 8.24 (br s, 1H), 9.24 (s, 1H), 11.58 (s, 1H)

### Example 44

### 3-(4-(3-Amino-1H-indazol-5-yl)pyridin-2-yl)-1-(2-hydroxyethyl)-1-methylurea

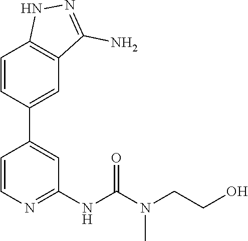 [SMILES: CN(CCO)C(=O)Nc1cc(-c2ccc3[nH]nc(N)c3c2)ccn1]

Ethyl (4-(3-amino-1H-indazol-5-yl)pyridin-2-yl)carbamate (Example 35) (0.030 g, 0.10 mmol) and 2-(methylamino)ethanol (0.5 ml) were placed in a 0.5-2 ml microwave vial which was then sealed and heated to 140° C. for 40 min. The solution was then allowed to cool and the solvent removed under reduced pressure. The crude residue was then dissolved in DMF (0.5 ml) and purified by HPLC to give the desired product. (0.007 g, 0.02 mmol, 16%) δ_{H} (d₆-DMSO): 3.04 (s, 3H), 3.48 (t, J=5.3 Hz, 2H), 3.62 (t, J=5.4 Hz, 2H) 7.42 (d, J=8.8 Hz, 2H), 7.52 (d, J=5.3 Hz, 1H), 7.68 (dd, J=8.5 and 1.5 Hz, 1H) 8.02 (d, J=1.3 Hz, 1H), 8.27 (d, J=6.1 Hz, 1H), 8.32 (s, 1H), 9.85 (s, 1H). HRMS: C₁₆H₁₈N₆O₂ requires 326.1491, found 327.1564 (M+H)⁺.

### Example 45

### 1-(4-(3-Amino-1H-indazol-5-yl)pyridin-2-yl)-3-benzylurea

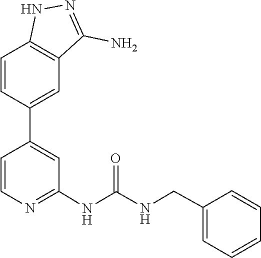 [SMILES: Nc1n[nH]c2ccc(-c3ccnc(NC(=O)NCc4ccccc4)c3)cc12]

1-Benzyl-3-(4-bromopyridin-2-yl)urea (0.161 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.034 g, 0.10 mmol, 18%) δ_{H} (d₆-DMSO): 4.41 (d, J=5.5 Hz, 2H), 7.23 (m, 1H), 7.26 (m, 5H), 7.42 (d, J=9.0 Hz, 1H), 7.64 (dd, J=8.8 and 1.4 Hz, 1H), 7.72 (s, 1H), 8.22 (m, 2H), 8.38 (s, 1H), 9.70 (s, 1H). HRMS: C₂₀H₁₈N₆O requires 358.1542, found 359.1615 (M+H)⁺.

### Example 46

### 1-(4-(3-Amino-1H-indazol-5-yl)pyridin-2-yl)-3-phenethylurea

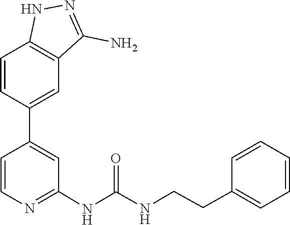 [SMILES: Nc1n[nH]c2ccc(-c3ccnc(NC(=O)NCCc4ccccc4)c3)cc12]

1-(4-Bromopyridin-2-yl)-3-phenethylurea (0.169 g, 0.53 mmol) along with 2-fluoro-5-(4,4,5,5-tetramethyl-1,3,2-dioxaborolan-2-yl)benzonitrile (0.132 g, 0.53 mmol), and t-butylamine (150 μl) were placed in a 5 ml microwave vial to which IPA:H₂O (2:1) (4 ml) was added. The solution was then degassed with N₂ for 10 min. [1,1′-bis(di-tert-butylphosphino)ferrocene]dichloropalladium(II) catalyst (0.020 g, 0.03 mmol, 5 mol %) was then added and the vial sealed and heated to 140° C. for 40 min. The solution was then allowed to cool before hydrazine hydrate (1 ml) was added and the solution heated to 100° C. for 1 hr in the microwave. The solution was then allowed to cool before removing the solvent under reduced pressure to give the crude product which was purified by HPLC to give the desired product. (0.023 g, 0.06 mmol, 12%) δ_{H} (d₆-DMSO): 2.81 (t, J=7.0 Hz, 2H), 3.44 (q, J=5.5 Hz, 2H), 7.27 (m, 6H), 7.44 (d, J=9.0 Hz, 1H), 7.68 (m, 2H), 8.19 (d, J=7.5 Hz, 1H), 8.27 (s, 1H), HRMS: C₂₁H₂₀N₆O requires 372.1699, found 373.1771 (M+H)⁺.

### Example 47

1-(4-(3-Amino-1H-indazol-5-yl)pyridin-2-yl)-3-(pyridin-2-ylmethyl)urea

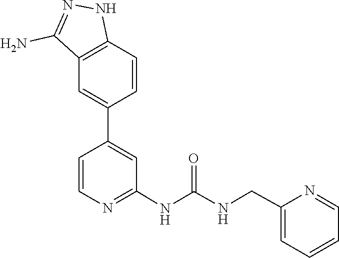 [SMILES: Nc1n[nH]c2ccc(-c3ccnc(NC(=O)NCc4ccccn4)c3)cc12]

A 0.5-2 mL microwave vial was charged with ethyl (4-(3-amino-1H-indazol-5-yl)pyridin-2-yl)carbamate (Example 35) (0.030 g, 0.10 mmol, 1 eq.), pyridin-2-ylmethanamine (0.044 g, 41.6 μL, 0.40 mmol, 4 eq.) and dioxane (650 μL), was sealed with an aluminium crimp cap fitted with a disposable PTFE/silicon septum, and purged with nitrogen. The reaction mixture was heated to 180° C. under microwave irradiation for 40 minutes. The reaction mixture was then concentrated in vacuo and the resulting crude was dissolved in water (3 mL). The mixture was stirred and sonicated for 30 minutes, then the solid was filtered, washed with water (2×3 mL) and dried to afford the title compound. (30 mg, 0.08 mmol, 83%) ¹H NMR (400 MHz, DMSO-D₆) δ 4.52 (d, J=5.5 Hz, 2H), 5.53 (s, 2H), 7.23 (d, J=5.5 Hz, 1H), 7.31-7.25 (m, 1H), 7.35 (t, J=7.7 Hz, 2H), 7.55 (d, J=8.8 Hz, 1H), 7.72 (s, 1H), 7.78 (t, J=7.7 Hz, 1H), 8.13 (br s, 1H), 8.23 (d, J=5.4 Hz, 1H), 8.54 (s, 1H), 8.78 (s, 1H), 9.41 (s, 1H), 11.58 (s, 1H). HRMS: Calculated for C₁₉H₁₈ON₇ 360.1567 (M+H⁺); Found: 360.1564

### Example 48

### 1-(4-(3-Amino-1H-indazol-5-yl)pyridin-2-yl)-3-(pyridin-3-ylmethyl)urea

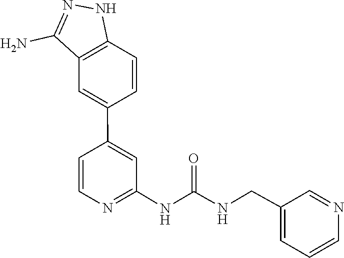 [SMILES: Nc1n[nH]c2ccc(-c3ccnc(NC(=O)NCc4cccnc4)c3)cc12]

A 0.5-2 mL microwave vial was charged with ethyl (4-(3-amino-1H-indazol-5-yl)pyridin-2-yl)carbamate (EXAMPLE 35) (0.030 g, 0.10 mmol, 1 eq.), pyridin-3-ylmethanamine (0.044 g, 41.6 μL, 0.40 mmol, 4 eq.) and dioxane (650 μL), was sealed with an aluminium crimp cap fitted with a disposable PTFE/silicon septum, and purged with nitrogen. The reaction mixture was heated to 180° C. under microwave irradiation for 40 minutes. The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound. (29 mg, 0.08 mmol, 81%) ¹H NMR (400 MHz, DMSO-D₆) δ 4.45 (d, J=5.9 Hz, 2H), 5.53 (s, 2H), 7.23 (d, J=5.4 Hz, 1H), 7.42-7.30 (m, 2H), 7.54 (d, J=8.7 Hz, 1H), 7.69 (s, 1H), 7.74 (d, J=7.7 Hz, 1H), 8.12 (s, 1H), 8.21 (d, J=5.4 Hz, 1H), 8.47 (d, J=4.7 Hz, 1H), 8.56 (s, 1H), 8.69 (br s, 1H), 9.37 (s, 1H), 11.59 (s, 1H). HRMS: Calculated for C₁₉H₁₈ON₇(M+H⁺): 360.1567; Found: 360.1564

### Example 49

### 1-(4-(3-Amino-1H-indazol-5-yl)pyridin-2-yl)-3-(pyridin-4-ylmethyl)urea

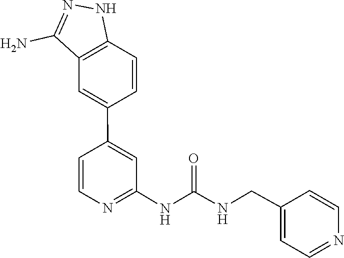 [SMILES: Nc1n[nH]c2ccc(-c3ccnc(NC(=O)NCc4ccncc4)c3)cc12]

A 0.5-2 mL microwave vial was charged with ethyl (4-(3-amino-1H-indazol-5-yl)pyridin-2-yl)carbamate (EXAMPLE 35) (0.030 g, 0.101 mmol, 1 eq.), pyridin-4-ylmethanamine (0.044 g, 41.6 μL, 0.40 mmol, 4 eq.) and dioxane (650 μL), was sealed with an aluminium crimp cap fitted with a disposable PTFE/silicon septum, and purged with nitrogen. The reaction mixture was heated to 180° C. under microwave irradiation for 40 minutes. The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound. (32 mg, 0.09 mmol, 89%) ¹H NMR (400 MHz, DMSO-D₆) 6 4.46 (d, J=6.0 Hz, 2H), 5.53 (s, 2H), 7.24 (d, J=5.3 Hz, 1H), 7.38-7.28 (m, 3H), 7.54 (d, J=9.5 Hz, 1H), 7.69 (s, 1H), 8.12 (s, 1H), 8.23 (d, J=5.4 Hz, 1H), 8.51 (d, J=5.8 Hz, 2H), 8.77 (br s, 1H), 9.44 (s, 1H), 11.58 (s, 1H).

### Example 50

### 1-(4-(3-Amino-1H-indazol-5-yl)pyridin-2-yl)-3-phenylurea

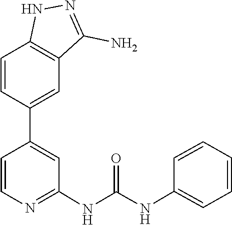 [SMILES: Nc1n[nH]c2ccc(-c3ccnc(NC(=O)Nc4ccccc4)c3)cc12]

Ethyl (4-(3-amino-1H-indazol-5-yl)pyridin-2-yl)carbamate (EXAMPLE 35) (0.050 g, 0.17 mmol) and analine (16 μl, 0.20 mmol, 1.2 eq) were placed in a 0.5-2 ml microwave vial with dioxane (2 ml). The solution was then degassed with nitrogen for 5 min, before bismuth triflate (0.006 g, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (5 mg, 0.02 mmol, 9%) δ_{H} (d₆-DMSO): 7.01 (t, J=7.5 Hz, 1H), 7.30 (m, 3H), 7.40 (d, J=9.0 Hz, 1H), 7.54 (d, J=7.5 Hz, 2H), 7.64 (dd, J=8.5 and 1.5 Hz, 1H), 7.85 (s, 1H), 8.21 (s, 1H), 8.32 (d, J=5.5 Hz, 1H), 9.50 (s, 1H), 10.38 (s, 1H). HRMS: C₁₉H₁₆N₆O requires 344.1386, found 345.1458 (M+H)⁺.

### Example 51

### 1-(4-(3-Amino-1H-indazol-5-yl)pyridine-2-yl)-3-(3-fluorophenyl)urea

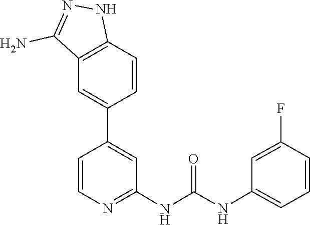 [SMILES: Nc1n[nH]c2ccc(-c3ccnc(NC(=O)Nc4cccc(F)c4)c3)cc12]

A suspension of ethyl (4-(3-amino-1H-indazol-5-yl)pyridin-2-yl)carbamate (0.050 g, 0.17 mmol), 3-fuoroanaline (60 μL), in dioxane (3 ml) was degassed with nitrogen. Bismuth triflate (0.008 g) was added and the mixture was heated in the microwave at 120° C. for 80 min. The reaction mixture was concentrated and the residue purifiyed by HPLC to afforded the title compound as a yellow off-white solid (0.008 g, 0.02 mmol, 13%). ¹H NMR (400 MHz, DMSO-d₆): δ 5,55 (s, 2H), 6.85 (td, J=8.0, and 2.0 Hz, 1H), 7.23 (dd, J=7.9, and 1.9 Hz, 1H), 7.35 (m, 3H), 7.60 (td, J=7.7, and 1.80 Hz, 1H), 7.86 (s, 1H), 8.21 (s, 1H), 8.34 (d, J=5.6 Hz, 1H), 10.67 (s, 1H), 11.80 (s, 1H). LRMS: C₁₉H₁₆N₆O requires 362.13, found 363.07 (M+H)⁺.

### Example 52

### 1-(4-(3-Amino-1H-indazol-5-yl)pyridin-2-yl)-3-(3-chlorophenyl)urea

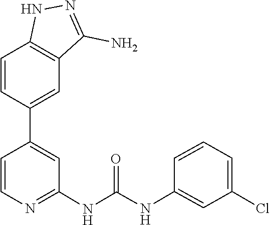 [SMILES: Nc1n[nH]c2ccc(-c3ccnc(NC(=O)Nc4cccc(Cl)c4)c3)cc12]

Ethyl (4-(3-amino-1H-indazol-5-yl)pyridin-2-yl)carbamate (EXAMPLE 35) (0.050 g, 0.17 mmol) and 3-chloroanaline (17 μl, 0.20 mmol, 1.2 eq) were placed in a 0.5-2 ml microwave vial with dioxane (2 ml). The solution was then degassed with nitrogen for 5 min, before bismuth triflate (0.006 g, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.006 g, 0.01 mmol, 6%) δ_{H} (d₆-DMSO): 6.97 (d, J=7.6 Hz, 1H), 7.23 (t, J=8.0 Hz, 1H), 7.32 (d, J=5.6 Hz, 1H), 7.40 (m, 2H), 7.52 (s, 1H), 7.66 (dd, J=8.5 and 1.5 Hz, 1H), 7.87 (s, 1H), 8.22 (s, 1H), 8.32 (d, J=5.6 Hz, 1H), 9.50 (s, 1H), 10.34 (s, 1H). HRMS: C₁₉H₁₅ClN₆O requires 378.0996, found 379.1069 (M+H)⁺.

### Example 53

### 1-(4-(3-Amino-1H-indazol-5-yl)pyridin-2-yl)-3-(3-isopropyl)phenyl)urea

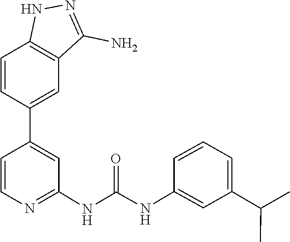 [SMILES: CC(C)c1cccc(NC(=O)Nc2cc(-c3ccc4[nH]nc(N)c4c3)ccn2)c1]

A suspension of ethyl [4-(3-amino-1H-indazol-5-yl)pyridin-2-yl]carbamate (EXAMPLE 35) (0.050 g, 0.17 mmol), bismuth(Ill)trifluoromethanesulfonate (0.030 g, 0.05 mmol), 3-isopropoxyaniline (0.050 mL, 0.36 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=22 min) to afford the title compound as the TFA salt (0.0018 g, 0.005 mmol, 2.9%). ¹H NMR (DMSO-d₆): δ 1.23 (d, J=6.8 Hz, 6H), 2.88 (m, 1H), 6.93 (d, J=7.2 Hz, 1H), 7.24 (t, J=8.0 Hz, 1H), 7.34 (m, 2H), 7.40 (m, 2H), 7.65 (dd, J=8.8, 1.2 Hz, 1H), 7.88 (s, 1H), 8.22 (s, 1H), 8.33 (d, J=5.6 Hz, 1H), 9.46 (s, 1H), 10.34 (s, 1H), 11.84 (br s, 1H). HRMS: For C₂₂H₂₃N₆O requires 387.1928, found 387.1923.

### Example 54

### 1-(4-(3-Amino-1H-indazol-5-yl)pyridin-2-yl)-3-(3-(hydroxymethyl)phenyl)urea

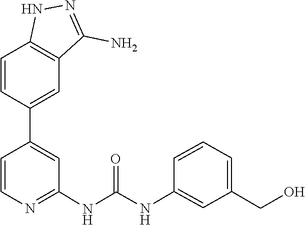 [SMILES: Nc1n[nH]c2ccc(-c3ccnc(NC(=O)Nc4cccc(CO)c4)c3)cc12]

Ethyl (4-(3-amino-1H-indazol-5-yl)pyridin-2-yl)carbamate (EXAMPLE 35) (0.050 g, 0.17 mmol) and (3-aminophenyl)methanol (0.025 g, 0.20 mmol, 1.2 eq) were placed in a 0.5-2 ml microwave vial with dioxane (2 ml). The solution was then degassed with nitrogen for 5 min, before bismuth triflate (0.006 mg, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.023 g, 0.06 mmol, 35%) δ_{H} (d₆-DMSO): 4.49 (2H, s), 6.97 (1H, d, J=7.6 Hz), 7.23 (1H, t, J=8.0 Hz), 7.32 (1H, d, J=5.6 Hz), 7.40 (2H, m), 7.52 (1H, s), 7.66 (1H, dd, J=8.5 and 1.5 Hz), 7.87 (1H, s), 8.22 (1H, s), 8.32 (1H, d, J=5.6 Hz), 9.50 (1H, s), 10.34 (1H, s). HRMS: C₂₀H₁₈N₆O₂ requires 374.1491, found 375.1566 (M+H)⁺.

### Example 55

### 3-(3-(4-(3-Amino-1H-indazol-5-yl)pyridin-2-yl)ureido)benzamide

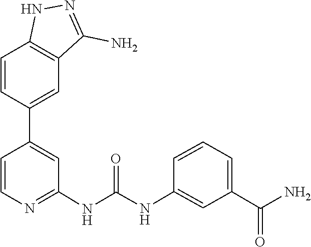 [SMILES: NC(=O)c1cccc(NC(=O)Nc2cc(-c3ccc4[nH]nc(N)c4c3)ccn2)c1]

Ethyl (4-(3-amino-1H-indazol-5-yl)pyridin-2-yl)carbamate (EXAMPLE 35) (0.050 g, 0.17 mmol) and 3-aminobenzamide (0.027 g, 0.20 mmol, 1.2 eq) were placed in a 0.5-2 ml microwave vial with dioxane (2 ml). The solution was then degassed with nitrogen for 5 min, before bismuth triflate (0.006 g, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.006 g, 0.02 mmol, 9%) δ_{H} (d₆-DMSO): 7.32 (m, 4H), 7.59 (d, J=8.9 Hz, 1H), 7.69 (dd, J=8.5 and 1.5 Hz, 1H), 7.80 (s, 1H), 8.19 (s, 1H), 8.33 (d, J=5.5 Hz, 1H), 9.55 (s, 1H), 10.58 (s, 1H). HRMS: C₂₀H₁₇N₇O₂ requires 387.1444, found 388.1516 (M+H)⁺.

### Example 56

### 1-(4-(3-Amino-1H-indazol-5-yl)pyridin-2-yl)-3-(3-phenoxyphenyl)urea

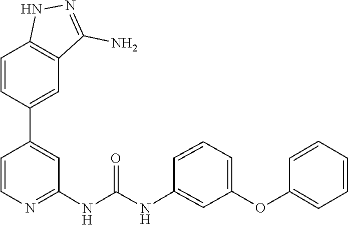 [SMILES: Nc1n[nH]c2ccc(-c3ccnc(NC(=O)Nc4cccc(Oc5ccccc5)c4)c3)cc12]

A suspension of ethyl [4-(3-amino-1H-indazol-5-yl)pyridin-2-yl]carbamate (EXAMPLE 35) (0.051 g, 0.17 mmol), bismuth(Ill)trifluoromethanesulfonate (0.037 g, 0.056 mmol), 3-phenoxyaniline (0.068 g, 0.37 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=23 min) to afford the title compound as the TFA salt (0.0022 g, 0.005 mmol, 2.9%). ¹H NMR (DMSO-d₆): δ 5.85 (t, J=2.4 Hz, 1H), 5.90 (t, J=0.6 Hz, 1H), 6.49 (br s, 3H), 6.66 (dd, J=1.6, 7.2 Hz, 1H), 7.04 (dd, J=1.2, 8.8 Hz, 2H), 7.15 (t, J=7.4 Hz, 1H), 7.22 (m, 1H), 7.38 (m, 5H), 7.58 (dd, J=1.6, 8.8 Hz, 1H), 7.84 (s, 1H), 8.16 (s, 1H), 8.30 (d, J=5.6 Hz, 1H), 9.40 (s, 1H), 10.52 (br s, 1H), 11.60 (br s, 1H). HRMS: For C₂₅H₂₁O₂N₆ requires 437.1721 found 437.1717 and C₁₀H₁₁O₂N₄ requires 219.0877 found 219.0895 (half mass).

### Example 57

### 1-(4-(3-Amino-1H-indazol-5yl)pyridin-2-yl)-3-(3-(benzyloxy)phenyl)urea

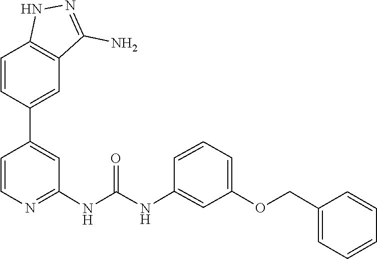 [SMILES: Nc1n[nH]c2ccc(-c3ccnc(NC(=O)Nc4cccc(OCc5ccccc5)c4)c3)cc12]

A suspension of ethyl [4-(3-amino-1H-indazol-5-yl)pyridin-2-yl]carbamate (EXAMPLE 35) (0.055 g, 0.19 mmol), bismuth(Ill)trifluoromethanesulfonate (0.034 g, 0.05 mmol), 3-benzyloxyaniline (0.048 g, 0.24 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=23 min) to afford the title compound as the TFA salt (0.0020 g, 0.004 mmol, 2.4%). ¹H NMR (DMSO-d₆): δ 5.10 (s, 2H), 6.70 (dd, J=7.6, 2.0 Hz, 1H), 7.05 (dd, J=7.6, 1.2 Hz, 1H), 7.22 (t, J=8.2 Hz, 1H), 7.35 (m, 3H), 7.40 (m, 3H), 7.46 (m, 2H), 7.64 (dd, J=8.8, 1.6 Hz, 1H), 7.85 (s, 1H), 8.21 (s, 1H), 8.32 (d, J=5.6 Hz, 1H), 9.50 (s, 1H), 10.42 (s, 1H), 11.84 (br s, 1H). HRMS: For C₂₆H₂₃N₆O₂ requires 451.1877, found 451.1872.

### Example 58

### 1-(4-(3-Amino-1H-indazol-5-yl)pyridin2-yl)-3-(3-((4-fluorobenzyl)oxy)phenyl)urea

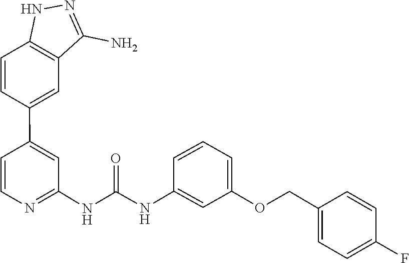 [SMILES: Nc1n[nH]c2ccc(-c3ccnc(NC(=O)Nc4cccc(OCc5ccc(F)cc5)c4)c3)cc12]

A suspension of ethyl [4-(3-amino-1H-indazol-5-yl)pyridin-2-yl]carbamate (EXAMPLE 35) (0.056 g, 0.19 mmol), bismuth(Ill)trifluoromethanesulfonate (0.052 g, 0.08 mmol), 3-(4-fluorobenzyloxy)phenylamine (0.21 mL, 0.96 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=25 min) to afford the title compound as the TFA salt (0.0057 g, 0.012 mmol, 6.5%). ¹H NMR (DMSO-d₆): δ 5.08 (s, 2H), 6.70 (m, 1H), 7.05 (m, 1H), 7.23 (m, 3H), 7.34 (m, 2H), 7.41 (m, 1H), 7.52 (m, 2H), 7.66 (d, J=7.6 Hz, 1H), 7.86 (s, 1H), 8.22 (s, 1H), 8.33 (d, J=4.4 Hz, 1H), 9.52 (br s, 1H), 10.44 (br s, 1H), 11.85 (br s, 1H). HRMS: For C₂₆H₂₂O₂N₆F requires 469.1783 found 469.1780.

### Example 59

### 1-(4-(3-Amino-1H-indazol-5-yl)pyridin-2-yl)-3-(3-((3-fIuorobenzyl)oxy)phenyl)urea

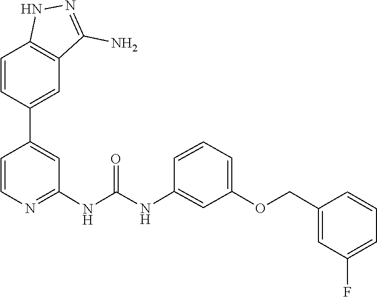 [SMILES: Nc1n[nH]c2ccc(-c3ccnc(NC(=O)Nc4cccc(OCc5cccc(F)c5)c4)c3)cc12]

A suspension of ethyl [4-(3-amino-1H-indazol-5-yl)pyridin-2-yl]carbamate (EXAMPLE 35) (0.054 g, 0.18 mmol), bismuth(Ill)trifluoromethanesulfonate (0.050 g, 0.08 mmol), 3-(3-fluorobenzyloxy)phenylamine (0.20 g, 0.91 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=24 min) to afford the title compound as the TFA salt (0.0032 g, 0.007 mmol, 3.8%). ¹H NMR (DMSO-d₆): δ 5.13 (s, 2H), 6.70 (dd, J=2.0, 7.6 Hz, 1H), 7.06 (m, 1H), 7.17 (m, 1H), 7.23 (t, J=8.2 Hz, 2H), 7.32 (m, 4H), 7.40 (d, J=8.8 Hz, 1H), 7.45 (m, 1H), 7.63 (dd, J=1.6, 8.8 Hz, 1H), 7.85 (br s, 1H), 8.20 (s, 1H), 8.32 (d, J=5.2 Hz, 1H), 9.50 (br s, 1H), 10.47 (br s, 1H), 11.80 (br s, 1H). HRMS: For C₂₆H₂₂O₂N₆F requires 469.1783 found 469.1781.

### Example 60

### 1-(4-(3-Amino-1H-indazol-5-yl)pyridin-2-yl)-3-(3-((2-fluorobenzyl)oxy)phenyl)urea

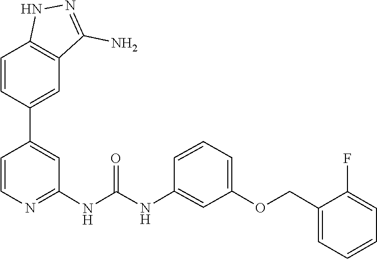 [SMILES: Nc1n[nH]c2ccc(-c3ccnc(NC(=O)Nc4cccc(OCc5ccccc5F)c4)c3)cc12]

A suspension of ethyl [4-(3-amino-1H-indazol-5-yl)pyridin-2-yl]carbamate (EXAMPLE 35) (0.065 g, 0.22 mmol), bismuth(Ill)trifluoromethanesulfonate (0.061 g, 0.09 mmol), 3-(2-fluorobenzyloxy)phenylamine (0.22 g, 1.01 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=25 min) to afford the title compound as the TFA salt (0.00656 g, 0.014 mmol, 6.4%). ¹H NMR (DMSO-d₆): δ 5.14 (s, 2H), 6.72 (dd, J=2.0, 8.0 Hz, 1H), 7.07 (m, 1H), 7.25 (m, 3H), 7.34 (m, 2H), 7.43 (m, 2H), 7.58 (dt, J=1.6, 7.6 Hz, 1H), 7.65 (dd, J=1.6, 8.8 Hz, 1H), 7.85 (s, 1H), 8.22 (s, 1H), 8.33 (d, J=5.6 Hz, 1H), 9.52 (br s, 1H), 10.44 (br s, 1H). HRMS: For C₂₆H₂₂O₂N₆F requires 469.1783 found 469.1779.

### Example 61

### 3-(3-(4-(3-Amino-1H-indazol-5-yl)pyridine-2-yl)ureido)-N-phenylbenzamide

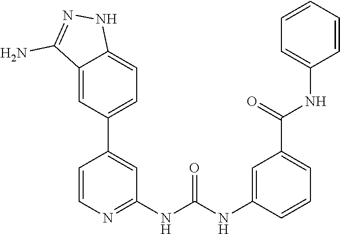 [SMILES: Nc1n[nH]c2ccc(-c3ccnc(NC(=O)Nc4cccc(C(=O)Nc5ccccc5)c4)c3)cc12]

A suspension of 3-(3-(4-bromopyridin-2-yl)ureido)-N-phenylbenzamide (0.060 g, 0.13 mmol), 5-(4,4,5,5-tetramethyl-1,3,2-dioxaborolan-2-yl)-1H-indazol-3-amine (0.054 g, 0.19 mmol), tetrakis(triphenylphosphine)palladium(0) catalyst (0.010 g) in EtOH/H₂O (2:1 mL) was degassed with nitrogen. Potassium phosphate monobasic (0.050 g, 0.22 mmol) was added and the mixture was heated to 80° C. for 16 hrs. The reaction mixture was concentrated to give the crude product. Purification by HPLC afforded the title compound as an off-white solid (0.013 g, 0.02 mmol, 22%). ¹H NMR (400 MHz, DMSO-d₆): δ 4.50 (d, J=5.6 Hz, 2H), 5,56 (s, 2H), 7.4 (m, 7H), 7.38 (m, 2H), 7.60 (d, J=7.6 Hz, 1H), 7.86 (s, 1H), 8.03 (s, 1H), 8.18 (s, 1H), 8.32 (d, J=5.5 Hz, 1H), 9.06 (s, 1H), 9.56 (s, 1H), 10.68 (s, 1H), 11.61 (s, 1H). LRMS: C₁₉H₁₆N₆O requires 463.18, found 464.2 (M+H).

### Example 62

### 1-(4-(3-Amino-1H-indazol-5-yl)pyridine-2-yl)-3-(4-fluorophenyl)urea

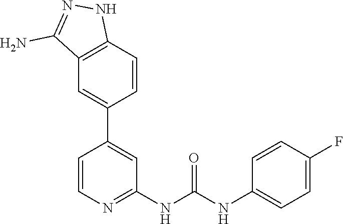 [SMILES: Nc1n[nH]c2ccc(-c3ccnc(NC(=O)Nc4ccc(F)cc4)c3)cc12]

A suspension of ethyl (4-(3-amino-1H-indazol-5-yl)pyridin-2-yl)carbamate (EXAMPLE 35) (0.050 g, 0.17 mmol), 4-fuoroanaline (60 μL), in dioxane (3 ml) was degassed with nitrogen. Bismuth triflate (0.008 g) was added and the mixture was heated in the microwave at 120° C. for 80 min. The reaction mixture was concentrated and the residue purifiyed by HPLC to afforded the title compound as a yellow off-white solid (0.005 g, 0.02 mmol, 13%). ¹H NMR (400 MHz, DMSO-d₆): δ 7.16 (m, 2H), 7.32 (d, J=5.52 Hz, 1H), 7.40 (d, J=8.44 Hz, 1H), 7.57 (m, 3H), 7.83 (s, 1H), 8.19 (s, 1H), 8.34 (d, J=5.5 Hz, 1H), 9.49 (s, 1H), 10.51 (s, 1H). LRMS: C₁₉H₁₆N₆O requires 362.13, found 363.13 (M+H)⁺.

### Example 63

### 1-(4-(3-Amino-1H-indazol-5-yl)pyridin-2-yl-3-(4-chlorophenyl)urea

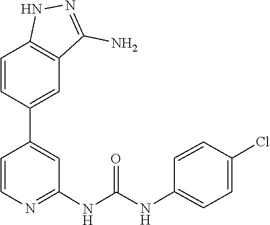 [SMILES: Nc1n[nH]c2ccc(-c3ccnc(NC(=O)Nc4ccc(Cl)cc4)c3)cc12]

Ethyl (4-(3-amino-1H-indazol-5-yl)pyridin-2-yl)carbamate (EXAMPLE 35) (0.050 g, 0.17 mmol) and 4-chloroaniline (0.026 g, 0.20 mmol, 1.2 eq) were placed in a 0.5-2 ml microwave vial with dioxane (2 ml). The solution was then degassed with nitrogen for 5 min, before bismuth triflate (0.006 g, 0.01 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.007 g, 0.02 mmol, 11%) δ_{H} (d₆-DMSO): 7.32 (m, 4H), 7.57 (d, J=8.9 Hz, 1H), 7.62 (dd, J=8.5 and 1.5 Hz, 1H), 7.84 (s, 1H), 8.20 (s, 1H), 8.32 (d, J=5.5 Hz, 1H), 9.54 (s, 1H), 10.55 (s, 1H). HRMS: C₁₉H₁₅ClN₆O requires 378.0996, found 379.1069 (M+H)⁺.

### Example 64

### 1-(4-(3-Amino-1H-indazol-5-yl)pyridin-2-yl)-3-(4-(tert-butyl)phenyl)urea

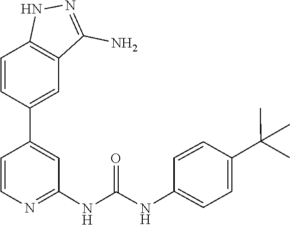 [SMILES: CC(C)(C)c1ccc(NC(=O)Nc2cc(-c3ccc4[nH]nc(N)c4c3)ccn2)cc1]

Ethyl (4-(3-amino-1H-indazol-5-yl)pyridin-2-yl)carbamate (EXAMPLE 35) (0.050 g, 0.17 mmol) and 4-t-butylaniline (32 μl, 0.20 mmol, 1.2 eq) were placed in a 0.5-2 ml microwave vial with dioxane (2 ml). The solution was then degassed with nitrogen for 5 min, before bismuth triflate (0.006 g, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.018 g, 0.05 mmol, 27%) δ_{H} (d₆-DMSO): 1.29 (s, 9H), 7.33 (m, 3H), 7.41 (d, J=8.8 Hz, 1H), 7.45 (d, J=6.8 Hz, 1H), 7.66 (dd, J=8.5 and 1.5 Hz, 1H), 7.86 (s, 1H), 8.22 (s, 1H), 8.32 (d, J=5.6 Hz, 1H), 9.47 (s, 1H), 10.29 (s, 1H). HRMS: C₂₃H₂₄N₆O requires 400.2012, found 401.2084 (M+H)⁺.

### Example 65

### 1-(4-(3-Amino-1H-indazol-5-yl)pyridin-2-yl)-3-(4-(methylsulfonyl)phenyl)urea

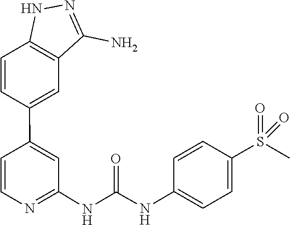 [SMILES: CS(=O)(=O)c1ccc(NC(=O)Nc2cc(-c3ccc4[nH]nc(N)c4c3)ccn2)cc1]

Ethyl (4-(3-amino-1H-indazol-5-yl)pyridin-2-yl)carbamate (EXAMPLE 35) (0.050 g, 0.17 mmol) and 4-(methylsulfonyl)aniline (0.034 g, 0.20 mmol, 1.2 eq) were placed in a 0.5-2 ml microwave vial with dioxane (2 ml). The solution was then degassed with nitrogen for 5 min, before bismuth triflate (0.006 g, 0.01 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.011 g, 0.03 mmol, 15%) δ_{H} (d₆-DMSO): 3.16 (s, 3H), 7.34 (dd, J=5.5 and 1.5 Hz, 1H), 7.40 (d, J=9.0 Hz, 1H), 7.64 (dd, J=9.0 and 1.5 Hz, 1H), 7.77 (d, J=8.5 Hz, 2H), 7.85 (d, J=8.5 Hz, 2H), 8.21 (s, 1H), 8.34 (d, J=5.5 Hz, 1H), 9.61 (s, 1H), 10.80 (s, 1H), 11.80 (s, 1H). HRMS: C₂₀H₁₈N₆O₃S requires 422.1161, found 423.1234 (M+H)⁺.

### Example 66

### 1-(4-(3-Amino-1H-indazol-5-yl)pyridin-2-yl)-3-(o-tolyl)urea

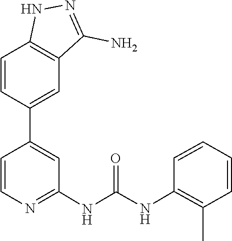 [SMILES: Cc1ccccc1NC(=O)Nc1cc(-c2ccc3[nH]nc(N)c3c2)ccn1]

A suspension of ethyl [4-(3-amino-1H-indazol-5-yl)pyridin-2-yl]carbamate (EXAMPLE 35) (0.060 g, 0.20 mmol), bismuth(Ill)trifluoromethanesulfonate (0.043 g, 0.067 mmol), o-toluidine (0.1 mL, 0.94 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=16 min) to afford the title compound as the TFA salt (0.0042 g, 0.011 mmol, 7.3%). ¹H NMR (DMSO-d₆): δ 2.35 (s, 3H), 6.98 (dt, J=7.4 Hz, 1H), 7.18 (t, J=7.4 Hz, 1H), 7.23 (d, J=7.2 Hz, 1H), 7.32 (dd, J=1.6, 5.6 Hz, 1H), 7.42 (d, J=8.8 Hz, 1H), 7.64 (m, 2H), 8.04 (d, J=7.6 Hz, 1H), 8.22 (s, 1H), 8.32 (d, J=5.2 Hz, 1H), 9.85 (br s, 1H), 10.89 (br s, 1H). HRMS: C₂₀H₁₉ON₆ requires 359.1615 found 359.1606.

### Example 67

### 1-(4-(3-Amino-1H-indazol-5-yl)pyridin-2-yl)-3-(2-ethylphenyl)urea

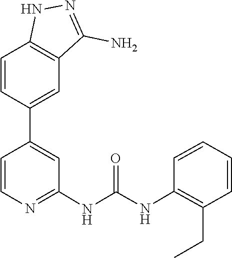 [SMILES: CCc1ccccc1NC(=O)Nc1cc(-c2ccc3[nH]nc(N)c3c2)ccn1]

A suspension of ethyl [4-(3-amino-1H-indazol-5-yl)pyridin-2-yl]carbamate (EXAMPLE 35) (0.054 g, 0.18 mmol), bismuth(Ill)trifluoromethanesulfonate (0.050 g, 0.08 mmol), 2-ethylaniline (0.11 mL, 0.89 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=18 min) to afford the title compound as the TFA salt (0.0043 g, 0.012 mmol, 6.4%). ¹H NMR (DMSO-d₆): δ 1.25 (t, J=7.6 Hz, 3H), 2.72 (q, J=7.5 Hz, 2H), 7.02 (dt, J=1.2, 7.6 Hz, 1H), 7.18 (dt, J=2.0, 8.0 Hz, 1H), 7.23 (dd, J=1.6, 7.6 Hz, 1H), 7.32 (dd, J=1.6, 5.6 Hz, 1H), 7.41 (d, J=8.8 Hz, 1H), 7.58 (br s, 1H), 7.62 (dd, J=1.6, 8.8 Hz, 1H), 8.05 (d, J=8.0 Hz, 1H), 8.20 (s, 1H), 8.31 (5.2 Hz, 1H), 9.84 (br s, 1H), 11.04 (br s, 1H), 11.85 (br s, 1H). HRMS: For C₂₁H₂₁ON₆ requires 373.1771 found 373.1773.

### Example 68

### 1-(4-(3-Amino-1H-indazol-5-yl)pyridin-2-yl)-3-(2-isopropylphenyl)urea

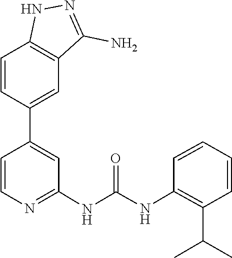 [SMILES: CC(C)c1ccccc1NC(=O)Nc1cc(-c2ccc3[nH]nc(N)c3c2)ccn1]

A suspension of ethyl [4-(3-amino-1H-indazol-5-yl)pyridin-2-yl]carbamate (EXAMPLE 35) (0.053 g, 0.18 mmol), bismuth(Ill)trifluoromethanesulfonate (0.055 g, 0.08 mmol), 2-isopropylaniline (0.13 mL, 0.9 mmol) in 1,4-dioxane (4 mL) was degassed for 5 min using a steady stream of nitrogen. The reaction mixture was reacted in the microwave at 120° C. for 1 h prior to being cooled to room temperature and concentrated under reduced pressure. Crude reaction mixture was taken up in DMF (0.5 mL) and purified by HPLC (t_{R}=20 min) to afford the title compound as the TFA salt (0.002 g, 0.005 mmol, 2.9%). ¹H NMR (DMSO-d₆): δ 1.28 (d, J=6.8 Hz, 6H), 3.24 (m, 1H), 7.10 (m, 1H), 7.18 (m, 1H), 7.31 (m, 1H), 7.33 (s, 1H), 7.41 (d, J=8.8 Hz, 1H), 7.59 (s, 1H), 7.62 (dd, J=1.6, 8.8 Hz, 1H), 7.95 (d, J=7.2 Hz, 1H), 8.20 (s, 1H), 8.29 (d, J=5.6 Hz, 1H), 9.82 (s, 1H), 11.01 (br s, 1H), 11.79 (br s, 1H). HRMS: For C₂₂H₂₃ON₆ requires 387.1928 found 387.1922.

### Example 69

### 1-(4-(3-Amino-1H-indazol-5-yl)pyridin-2-yl)-3-(pyridin-3-yl)urea

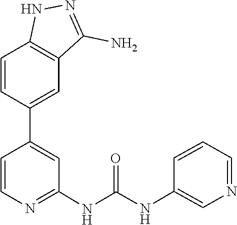 [SMILES: Nc1n[nH]c2ccc(-c3ccnc(NC(=O)Nc4cccnc4)c3)cc12]

Ethyl (4-(3-amino-1H-indazol-5-yl)pyridin-2-yl)carbamate (EXAMPLE 35) (0.050 g, 0.17 mmol) and 3-aminopyridine (0.019 g, 0.20 mmol, 1.2 eq) were placed in a 0.5-2 ml microwave vial with dioxane (2 ml). The solution was then degassed with nitrogen for 5 min, before bismuth triflate (0.006 g, 0.009 mmol, 5 mol %) was added, the vial was then sealed and heated to 120° C. for 60 min. The solution was allowed to cool and the solvent removed under reduced pressure. The crude residue was then dissolved in DMF (0.5 ml) and purified by HPLC to give the desired product. (0.004 g, 0.01 mmol, 7%) δ_{H} (d₆-DMSO): 7.36 (dd, J=5.6 and 1.6 Hz, 1H), 7.43 (d, J=8.4 Hz, 1H), 7.63 (m, 3H), 7.84 (s, 1H), 8.22 (s, 1H), 8.35 (d, J=5.6 Hz, 1H), 8.91 (d, J=2.0 Hz, 1H), 9.77 (s, 1H), 10.89 (s, 1H). LRMS: found: 346.00 (M+1), calculated C₁₈H₁₅N₇O: 345.13. HRMS: C₁₈H₁₅N₇O requires 345.1338, found 346.1352 (M+H)⁺.

### Section 6—Compounds of the Formula

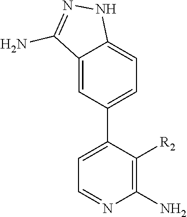

General procedure A:

A suspension of required chloroaryl (1 eq.), 5-(4,4,5,5-tetramethyl-1,3,2-dioxaborolan-2-yl)-1H-indazol-3-amine (1.2-1.5 eq.), base (2 eq.) in 1:3 of solvent was deoxygenated with nitrogen in sealed tube. Then [1,1′-bis(di-tert-butylphosphino)ferrocene]dichloropalladium(II) catalyst (0.05 eq.) was added then the tube was sealed and the mixture allowed to stirred at 90-100° C. for 18 h. After the reaction was cooled to room temperature, EtOAc and water were added. Extracted organic layer was dried over magnesium sulfate and concentrated under reduced pressure and the residue purified by column chromatography.

### Example 70

### 4-Chloro-3-((triisopropylsilyl)ethynyl)pyridin-2-amine

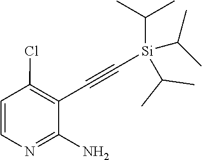 [SMILES: CC(C)[Si](C#Cc1c(Cl)ccnc1N)(C(C)C)C(C)C]

A solution of 4-chloro-3-iodopyridin-2-amine (1.00 g, 3.9 mmol), copper (1) iodide (38 mg, 0.20 mmol) and bis(triphenylphosphine) palladium(II) chloride (0.28 g, 0.4 mmol) in 16 ml of N,N-dimethyformamide-triethylamine (4:1) was degassed in sealed tube followed by addition of ethynyltriisopropylsilane (3.6 mL, 16.0 mmol). The reaction mixture was heated to 80° C., o/n. The resulting mixture was diluted with EtOAc, filtered through silica, concentrated under reduced pressure and the residue was purified by column chromatography (petroleum ether 60-80%/acetone 9:1) to give the product as a brown solid. (1.01 g, 84%). ¹H NMR (500 MHz, (CD₃)₂CO): δ 0.83-0.89 (m, 3H), 1.15-1.19 (m, 18H), 5.88 (br s, 2H), 6.74 (d, J=5.4 Hz, 1H), 7.92 (d, J=5.4 Hz, 1H). LRMS: Calculated for C₁₅H₂₅ClN₂Si 308.2 found 309.2 (M+1).

### 5-(2-amino-3-((triisopropylsilyl)ethynyl)pyridin-4-yl)-2-fluorobenzonitrile

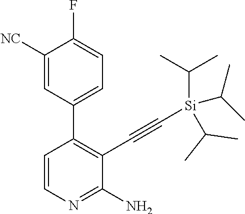 [SMILES: CC(C)[Si](C#Cc1c(-c2ccc(F)c(C#N)c2)ccnc1N)(C(C)C)C(C)C]

A solution of 4-chloro-3-((triisopropylsilyl)ethynyl)pyridin-2-amine (1.05 g, 3.9 mmol), K₃PO4 (5.40 g, 25.5 mmol), Sphos (0.14 g, 0.34 mmol), palladium(II) chloride (0.76 g, 0.34 mmol) and 2-fluoro-5-(4,4,5,5-tetramethyl-1,3,2-dioxaborolan-2-yl)benzonitrile (2.10 g, 8.5 mmol) in 22 ml of dioxane-water (5:1) was degassed in sealed tube and submitted to reaction. The reaction mixture was heated to 80° C. for 4 hours. The resulting mixture was diluted with EtOAc, filtered through silica, concentrated under reduced pressure and the residue was purified by column chromatography (petroleum ether 60-80%/acetone 7:3) to give the product as a brown solid. (0.62 g, 41%). ¹H NMR (500 MHz, (CD₃)₂CO): δ 1.05-1.09 (m, 21H), 5.84 (br s, 2H), 6.72 (d, J=5.2 Hz, 1H), 7.55 (app t, J_{H-H; H-F}=8.8 Hz, 1H), 8.00 (ddd, J_{H-H; H-F; H-H}=8.8, 5.2, 2.3 Hz, 1H), 8.07 (d, J=5.2 Hz, 1H), 8.09 (dd, J_{H-F; H-H}=6.2, 2.3 Hz, 1H). LRMS: Calculated for C₂₃H₂₈FN₃Si 393.2 found 394.1 (M+1).

### 5-(2-Amino-3-ethynylpyridin-4-yl)-2-fluorobenzonitrile

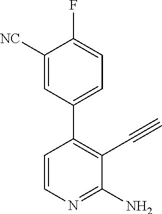 [SMILES: C#Cc1c(-c2ccc(F)c(C#N)c2)ccnc1N]

To a solution of 5-(2-amino-3-((triisopropylsilyl)ethynyl)pyridin-4-yl)-2-fluorobenzonitrile (0.6 g, 1.53 mmol) in 6 ml of THF was added TBAF (2.0 g, 7.7 mmol). The reaction was stirred at RT for 1 hour. The resulting mixture was diluted with EtOAc, filtered through silica, concentrated under reduced pressure and the residue purified by column chromatography (petroleum ether 60-80%/acetone 7:3) to give the product as a brown solid. (0.323 g, 89%) ¹H NMR (400 MHz, (CD₃)₂CO): 4.08 (s, 1H), 5.97 (br s, 2H), 6.71 (d, J=5.2 Hz, 1H), 7.55 (app t, J_{H-H; H-F}=9.0 Hz, 1H), 7.99-8.11 (m, 3H). LRMS: Calculated for C₁₄H₈FN₃ 237.1 found 238.1 (M+1). 5-(2-Amino-3-ethynylpyridin-4-yl)-1H-indazol-3-amine

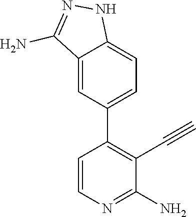 [SMILES: C#Cc1c(-c2ccc3[nH]nc(N)c3c2)ccnc1N]

To a solution of 5-(2-amino-3-ethynylpyridin-4-yl)-2-fluorobenzonitrile (90 mg, 0.4 mmol) in 0.5 ml of EtOH was added hydrazine (1.5 mL, 1 Mol solution in EtOH). The reaction was stirred at 80° C., o/n. The resulting mixture was diluted with EtOAc, washed with water, concentrated under reduced pressure and the residue purified by HPLC to give the product as a yellow solid. (11 mg, 11%) ¹H NMR (400 MHz, MeOD): δ 3.88 (s, 1H), 6.74 (d, J=5.4 Hz, 1H), 7.34 (dd, J=8.7, 0.8 Hz, 1H), 7.63 (dd, J=8.7, 1.7 Hz, 1H), 7.93 (d, J=5.4 Hz, 1H), 7.99 (dd, J=1.7, 0.8 Hz, 1H), 5 protons missing. LRMS: Calculated for C₁₄H₁₁N₅ 249.1 found 250.1 (M+1).

### Example 71

### 4-Chloro-3-(cyclopropylethynyl)pyridin-2-amine

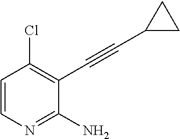 [SMILES: Nc1nccc(Cl)c1C#CC1CC1]

A solution of 4-chloro-3-iodopyridin-2-amine (1.00 g, 3.9 mmol), copper (1) iodide (38 mg, 0.2 mmol) and bis(triphenylphosphine) palladium(II) chloride (0.28 g, 0.4 mmol) in 16 ml of N,N-dimethyformamide-triethylamine (4:1) was degassed in sealed tube followed by addition of ethynylcyclopropane (1.35 mL, 16.0 mmol). The reaction mixture was heated to 80° C. for 5 hours. The resulting mixture was diluted with EtOAc, filtered through silica, concentrated under reduced pressure and the residue was purified by column chromatography (petroleum ether 60-80%/acetone 9:1) to give the product as a brown solid. (0.68 g, 91%). ¹H NMR (400 MHz, (CD₃)₂CO): δ 0.80-0.86 (m, 2H), 0.90-1.00 (m, 2H), 1.61 (tt, J=8.2, 5.0 Hz, 1H), 5.88 (br s, 2H), 6.65 (d, J=5.5 Hz, 1H), 7.82 (d, J=5.5 Hz, 1H). LRMS: Calculated for C₁₀H₉ClN₂ 192.1 found 193.1 (M+1).

### 5-(2-amino-3-(cyclopropylethynyl)pyridin-4-yl)-2-fluorobenzonitrile

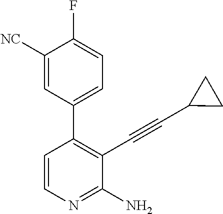 [SMILES: N#Cc1cc(-c2ccnc(N)c2C#CC2CC2)ccc1F]

A solution of 4-chloro-3-(cyclopropylethynyl)pyridin-2-amine (0.70 g, 3.64 mmol), K₃PO₄ (5.80 g, 27.3 mmol), Sphos (0.14 g, 0.34 mmol), palladium(II) acetate (76 mg, 0.34 mmol) and 2-fluoro-5-(4,4,5,5-tetramethyl-1,3,2-dioxaborolan-2-yl)benzonitrile (2.10 g, 8.5 mmol) in 22 ml of dioxane-water (5:1) was degassed in sealed tube and submitted to reaction. The reaction mixture was heated to 80° C. for 4 hours. The resulting mixture was diluted with EtOAc, filtered through silica, concentrated under reduced pressure and the residue was purified by column chromatography (petroleum ether 60-80%/acetone 7:3) to give the product as a dark yellow solid. (0.82 g, 81%). 1H NMR (500 MHz, (CD₃)₂CO): δ 0.68-0.73 (m, 2H), 0.85-0.90 (m, 2H), 1.49 (tt, J=8.2, 5.0 Hz, 1H), 5.83 (br s, 2H), 6.68 (d, J=5.2 Hz, 1H), 7.54 (app t, J_{H-H; H-F}=9.0 Hz, 1H), 7.98 (d, J=5.2 Hz, 1H), 8.01 (ddd, J_{H-H; H-F; H-H}=9.0, 5.2, 2.4 Hz, 1H), 8.08 (dd, J_{H-F; H-H}=6.2, 2.4 Hz, 1H). LRMS: Calculated for C₁₇H₁₂FN₃ 277.1 found 278.1 (M+1).

### 5-(2-Amino-3-(cyclopropylethynyl)pyridin-4-yl)-1H-indazol-3-amine

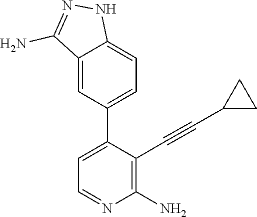 [SMILES: Nc1nccc(-c2ccc3[nH]nc(N)c3c2)c1C#CC1CC1]

To a solution of 5-(2-amino-3-(cyclopropylethynyl)pyridin-4-yl)-2-fluorobenzonitrile (75 mg, 0.19 mmol) in 0.3 ml of EtOH was added hydrazine (0.75 mL, 1 Mol solution in EtOH). The reaction was stirred at 80° C., o/n. The resulting mixture was diluted with EtOAc, washed with water, concentrated under reduced pressure and the residue purified by HPLC to give the product as a yellow solid. (21 mg, 38%) ¹H NMR (400 MHz, DMSO-d₆): δ 0.66-0.72 (m, 2H), 0.76-0.82 (m, 2H), 1.46 (tt, J=8.2, 5.0 Hz, 1H), 5.49 (br s, 2H), 6.06 (br s, 2H), 6.59 (d, J=5.2 Hz, 1H), 7.26 (dd, J=8.7, 0.8 Hz, 1H), 7.48 (dd, J=8.7, 1.7 Hz, 1H), 7.89 (d, J=5.2 Hz, 1H), 7.96 (dd, J=1.7, 0.8 Hz, 1H), 11.48 (br s, 1H). LRMS: Calculated for C₁₇H₁₅N₅ 289.1 found 290.2 (M+1).

### Example 72

### N-(4-Chloropyridin-2-yl)pivalamide

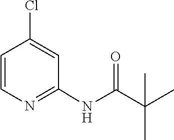

To mixture of 4-chloropyridin-2-amine (49 g, 38.61 mmol), HF 50 mL and triethylamine (13.47 mL, 96.53 mmol)) in a sealed flask flushed with argon and cooled to 0° C., was added pivaloyl chloride (5.58 g, 46.33 mmol) and stirred vigorously at this temperature for 1 hour, then allowed to warm up to room temperature overnight. The reaction was then diluted with EtOAC and partitioned with H₂O. The organic layer was collected and the aqueous layer extracted (2×30 mL). The combined organic layer was dried over anhydrous magnesium sulfate, concentrated and purified by flash column chromatography (50% EtOAc and 1% triethylamine in petroleum ether 60-80%) affording the target compound 6.4 g (78%). ¹H NMR (500 MHz, DMSO-d₆) δ 10.09 (s, 1H), 8.32 (d, J=5.4 Hz, 1H), 8.17 (d, J=1.8 Hz, 1H), 7.25 (dd, J=5.4, 1.9 Hz, 1H), 1.24 (s, 9H). LRMS (ESI) m/z [M]⁺. For C₁₀H₁₃ClN₂O Molecular weight 212.6770 found 213.5.

### N-(4-Chloro-3-iodopyridin-2-yl)pivalamide

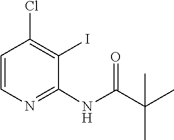

To a solution of N-(4-chloropyridin-2-yl)pivalamide (8.0 g, 37.72 mmol) in anhydrous THF flushed with argon and cooled to −78° C. was added n-BuLi (38 mL, 94.4 mmol of 2.5 M soln in hexanes). The resulting mixture was allowed to warm up to −20° C. and stirred for 1 hour before cooling back down to −78° C. and iodine (94.4 mmol) and TiMEDA (14.14 mL, 94.4 mmol) added. The reaction was then allowed to warm up to room temperature and stirred for a further 8 hours. The reaction was cooled to −78° C. and quenched by addition of NH₄Cl (aq). The reaction was then partitioned between EtOAC and water and the organic layer collected. The aqueous layer was extracted (2×30 ML) and the combined organic layer dried over anhydrous magnesium sulfate, concentrated and purified by flash column chromatography (50% EtOAc and 1% triethylamine in petroleum ether 60-80%) affording the target compound 5.7 g (45%). ¹H NMR (500 MHz, DMSO-d₆) δ 9.86 (s, 1H), 8.36 (d, J=5.2 Hz, 1H), 7.54 (d, J=5.2 Hz, 1H), 1.25 (s, 9H). LRMS (ESI) m/z [M]⁺. For C₁₀H₁₂ClIN₂O Molecular weight 338.5735 found 339.5

### 4-Chloro-3-iodopyridin-2-amine

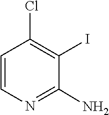

To a solution of N-(4-chloro-3-iodopyridin-2-yl)pivalamide (0.35 g, 1.03 mmol) in methanol was added 5M HCl (5 mL) and stirred at reflux overnight. The reaction was then concentrated and purified by flash column chromatography (50% EtOAc and 1% triethylamine in petroleum ether 60-80%) affording the target compound in quantitative yield. ¹H NMR (500 MHz, DMSO-d₆) δ 7.85 (d, J=5.2 Hz, 1H), 6.73 (d, J=5.2 Hz, 1H), 6.41 (s, 2H). LRMS (ESI) m/z [M]⁺. For C₅H₄ClN₂ Molecular weight 254.4555 found 255.3

### 4-Chloro-3-(3,3-dimethylbut-1-yn-1-yl)pyridin-2-amine

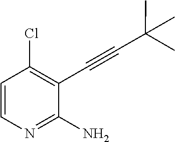

A solution of 4-chloro-3-iodo-pyridine-2-amine (0.3 g, 1.18 mmol) copper (I) iodide (0.0225 g, 0.118 mmol) and bis(triphenylphosphine) palladium(II) chloride (0.0829 g, 0.1181 mmol) in 3 ml of N,N-dimethyformamide-triethylamine (1:4) was degassed in sealed tube followed by addition of (0.1164 g, 0.173 mL, 1.417 mmol) of 3,3-dimethylbut-1-yne. The reaction mixture was stirred at 80° C. for 4 h. The reaction mixture was then diluted with EtOAc and washed with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown (0.267 g, Quant). ¹H NMR (500 MHz, DMSO-d₆) δ 7.84 (d, J=5.4 Hz, 1H), 6.68 (d, J=5.4 Hz, 1H), 6.31 (s, 2H), 1.33 (s, 9H). LRMS (ESI) m/z [M]⁺. For C₁₆H₁₃ClN₂ Molecular weight 208.689 found 209.3

### 5-(2-Amino-3-(3,3-dimethylbut-1-yn-1-yl)pyridin-4-yl)-2-fluorobenzonitrile

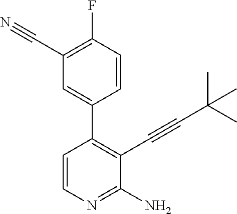 [SMILES: CC(C)(C)C#Cc1c(-c2ccc(F)c(C#N)c2)ccnc1N]

To a mixture of 4-chloro-3-(3,3-dimethylbut-1-yn-1-yl)pyridin-2-amine (0.267 g, 1.28 mmol, 1.0 eq), 2-fluoro-5-(4,4,5,5-tetramethyl-1,3,2-dioxaborolan-2-yl)benzonitrile (0.380 g, 1.54 mmol), [1,1′-bis(di-tert-butylphosphino)ferrocene]dichloropalladium(II) catalyst (0.042 g, 0.064 mmol, 5 mol %) and cesium carbonate (1.33 g, 4.106 mmol) in a sealed tube flushed with argon was added a degassed mixture of dioxane and water (18:2 3 mL) and stirred at 110° C. for 18 hours, concentrated and purified by flash chromatography (50% EtOAc and 1% triethylamine in petroleum ether 60-80%) to afford the desired product (0.105 g, 28%) ¹H NMR (500 MHz, DMSO-d₆) δ 8.20 (dd, J=6.3, 2.3 Hz, 1H), 8.03-7.96 (m, 2H), 7.65 (t, J=9.1 Hz, 1H), 6.67 (d, J=5.2 Hz, 1H), 6.19 (s, 2H), 1.22 (s, 9H).

### 5-(2-Amino-3-(3,3-dimethylbut-1-yn-1-yl)pyridine-4-yl)-1H-indazol-3-amine

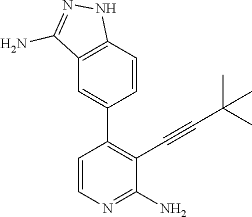 [SMILES: CC(C)(C)C#Cc1c(-c2ccc3[nH]nc(N)c3c2)ccnc1N]

5-(2-Amino-3-(3,3-dimethylbut-1-yn-1-yl)pyridin-4-yl)-2-fluorobenzonitrile (0.09 g, 0.3 mmol) was then dissolved in ethanol (8 ml) and hydrazine, (2 mL, 1 Mol solution in ethanol) added. The resulting mixture was stirred at reflux for 2 hours, cooled, concentrated and purified by HPLC affording the target compound as a yellow powder 0.028 g (26%). ¹H NMR (500 MHz, DMSO-de) δ 8.21 (d, J=1.6 Hz, 1H), 7.98 (d, J=6.6 Hz, 1H), 7.69 (dd, J=8.8, 1.8 Hz, 1H), 7.36 (d, J=8.7 Hz, 1H), 6.99 (d, J=6.6 Hz, 1H), 2.54-2.48 (m, 9H). LRMS (ESI) m/z [M]⁺. For C₁₈H₁₉N₅ Molecular Weight: 305.3850 found 306.3

### Example 73

### 4-Chloro-3-(cyclopentylethynyl)pyridin-2-amine

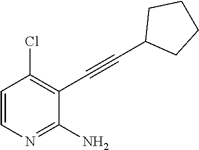 [SMILES: Nc1nccc(Cl)c1C#CC1CCCC1]

A solution of 4-chloro-3-iodo-pyridine-2-amine (0.15 g, 0.59 mmol), copper (I) iodide (0.0113 g, 0.059 mmo)l and bis(triphenylphosphine) palladium(II) chloride (g, 0.059 mmol) in 3 ml of N,N-dimethyformamide-triethylamine (1:4) was degassed in sealed tube followed by addition of ethynylcyclopentane (0.0667 g, 0.082 mL, 0.059 mmol). The reaction mixture was stirred at 80° C. for 4 h. The reaction mixture was then diluted with EtOAc and washed with 1M sodium carbonate. Extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown solid (0.061 g, 47%). ¹H NMR (500 MHz, DMSO-d₆) δ 7.83 (d, J=5.5 Hz, 1H), 6.67 (d, J=5.5 Hz, 1H), 6.33 (s, 2H), 2.97 (p, J=7.4 Hz, 1H), 1.97 (dddd, J=12.1, 7.1, 4.2, 1.4 Hz, 2H), 1.76-1.66 (m, 4H), 1.61-1.53 (m, 2H). LRMS (ESI) m/z [M]⁺. For C₁₂H₁₃ClN₂ Molecular weight 220.7000 found 221.1

### 5-(2-Amino-3-(cyclopentylethynyl)pyridin-4-yl)-2-fluorobenzonitrile

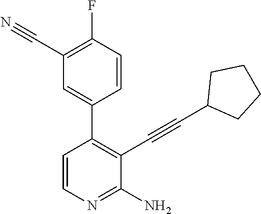 [SMILES: N#Cc1cc(-c2ccnc(N)c2C#CC2CCCC2)ccc1F]

To a mixture 4-chloro-3-(cyclopentylethynyl)pyridin-2-amine (0.061 g, 0.28 mmol, 1.0 eq), 2-fluoro-5-(4,4,5,5-tetramethyl-1,3,2-dioxaborolan-2-yl)benzonitrile (0.082 g, 0.33 mmol), [1,1′-bis(di-tert-butylphosphino)ferrocene]dichloropalladium(II) catalyst (0.009 g, 0.014 mmol, 5 mol %) and cesium carbonate (0.27 g, 0.832 mmol) in a sealed tube flushed with argon was added a degassed mixture of dioxane and water (18:2 3 mL) and stirred at 110° C. for 18 hours, concentrated and purified by flash chromatography to afford 5-(2-amino-3-(cyclopentylethynyl)pyridin-4-yl)-2-fluorobenzonitrile (0.105 g, 35%), ¹H NMR (500 MHz, DMSO-d₆) δ 8.17 (dd, J=6.3, 2.3 Hz, 1H), 8.03-7.95 (m, 2H), 7.64 (t, J=9.0 Hz, 1H), 6.65 (d, J=5.2 Hz, 1H), 6.21 (s, 2H), 2.87 (tt, J=8.3, 6.3 Hz, 1H), 1.87 (ddd, J=10.4, 5.2, 3.1 Hz, 2H), 1.64-1.50 (m, 6H).

### 5-(2-Amino-3-(cyclopentylethynyl)pyridine-4-yl)-1H-indazol-3-amine

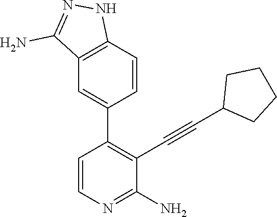 [SMILES: Nc1nccc(-c2ccc3[nH]nc(N)c3c2)c1C#CC1CCCC1]

5-(2-Amino-3-(cyclopentylethynyl)pyridin-4-yl)-2-fluorobenzonitrile (0.09 g, 0.295 mmol) was dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder 0.028 g (26%). ¹H NMR (500 MHz, DMSO-d₆) δ 8.20 (d, J=1.6 Hz, 1H), 7.98 (d, J=6.6 Hz, 1H), 7.81 (s, 2H), 7.66 (dd, J=8.7, 1.7 Hz, 1H), 7.35 (d, J=8.8 Hz, 1H), 6.98 (d, J=6.5 Hz, 1H), 2.91 (ddd, J=14.3, 7.6, 6.3 Hz, 1H), 1.88 (ddt, J=9.8, 7.7, 3.2 Hz, 2H), 1.66-1.44 (m, 6H). LRMS (ESI) m/z [M]⁺. For C₁₉H₁₆N₅ Molecular weight 317.396 found 318.3

### Example 74

### 4-Chloro-3-(cyclohexylethynyl)pyridin-2-amine

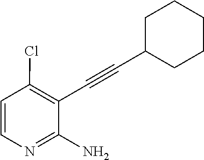 [SMILES: Nc1nccc(Cl)c1C#CC1CCCCC1]

A solution of 4-chloro-3-iodo-pyridine-2-amine (0.20 g/855 mmol) copper (1) iodide (0.010 g/0.051 mmol) and bis(triphenylphosphine) palladium(II) chloride (0.036 g/0.051 mmol) in 3 ml of N,N-dimethyformamide-triethylamine (1:4) was degassed in sealed tube followed by addition of ethynylcyclohexane (0.132 g/1.23 mmol) The reaction mixture was stirred at 80° C. for 4 h. The reaction mixture was then diluted with EtOAc and washed with 1M sodium carbonate. Extracted organic layer was washed with brine and dried over magnesium sulfate and concentrated under reduced pressure and the residue purified by column chromatography (50% EtOAc and 1% triethylamine in petroleum ether 60-80%) to give the product as a light brown solid(0.181 g 75%). ¹H NMR (500 MHz, DMSO-d6) δ=7.85 (d, J=5.8, 1H), 6.68 (d, J=5.3, 1H), 6.33 (s, 2H), 2.76 (tt, J=8.4, 3.9, 1H), 1.84 (ddd, J=13.0, 6.7, 3.2, 2H), 1.70 (ddp, J=10.7, 6.9, 3.8, 3.1, 2H), 1.61-1.43 (m, 3H), 1.43-1.30 (m, 3H). LRMS (ESI) m/z [M]⁺. For C₁₃H₁₅ClN₂ Molecular weight 234.7270 found 235.5.

### 5-(2-Amino-3-(cyclohexylethynyl)pyridin-4-yl)-2-fluorobenzonitrile

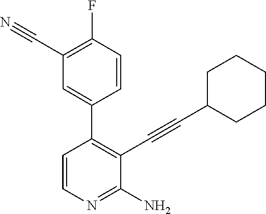 [SMILES: N#Cc1cc(-c2ccnc(N)c2C#CC2CCCCC2)ccc1F]

To a mixture 4-chloro-3-(cyclohexylethynyl)pyridin-2-amine (0.20 g, 0.854 mmol, 1.0 eq), 2-fluoro-5-(4,4,5,5-tetramethyl-1,3,2-dioxaborolan-2-yl)benzonitrile (0.0823 g, 1.025 mmol), [1,1′-bis(di-tert-butylphosphino)ferrocene]dichloropalladium(II) catalyst (0.055 g, 0.0845 mmol, 5 mol %), cesium carbonate (0.918 g, 2.818 mmol) in a sealed tube flushed with argon was added a degassed mixture of dioxane and water (18:2 5 mL) and stirred at 110° C. for 18 hours, concentrated and purified by flash chromatography (50% EtOAc and 1% triethylamine in petroleum ether 60-80%) to afford 5-(2-amino-3-(cyclohexylethynyl)pyridin-4-yl)-2-fluorobenzonitrile (0.195 g, 72%). ¹H NMR (400 MHz, DMSO-d6) δ 8.18 (s, 1H), 7.96 (d, J=6.5 Hz, 1H), 7.74 (s, 1H), 7.67 (d, J=8.9 Hz, 1H), 7.33 (d, J=8.8 Hz, 1H), 6.96 (d, J=6.5 Hz, 1H), 2.67 (dt, J=8.7, 4.7 Hz, 1H), 1.81-1.70 (m, 2H), 1.58-1.47 (m, 2H), 1.42 (dt, J=8.0, 3.4 Hz, 3H), 1.30-1.16 (m, 3H).

### 5-(2-Amino-3-(cyclohexylethynyl)pyridine-4-yl)-1H-indazol-3-amine

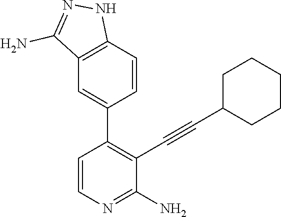 [SMILES: Nc1nccc(-c2ccc3[nH]nc(N)c3c2)c1C#CC1CCCCC1]

5-(2-Amino-3-(cyclohexylethynyl)pyridin-4-yl)-2-fluorobenzonitrile (0.1 g, 0.31 mmol) was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder 0.032 g (15%). ¹H NMR (500 MHz, DMSO-d₆) δ 8.21 (d, J=1.6 Hz, 1H), 7.99 (d, J=6.6 Hz, 1H), 7.88 (s, 2H), 7.69 (dd, J=8.7, 1.7 Hz, 1H), 7.35 (d, J=8.7 Hz, 1H), 6.99 (d, J=6.6 Hz, 1H), 2.68 (dt, J=9.2, 4.9 Hz, 1H), 1.75 (d, J=8.6 Hz, 2H), 1.56-1.48 (m, 2H), 1.44 (ddt, J=15.4, 10.9, 5.3 Hz, 3H), 1.29-1.18 (m, 3H). LRMS (ESI) m/z [M]⁺. For C₂₀H₂₁N₅ Molecular weight 331.423 found 332.3.

### Example 75

### 4-Chloro-3-(phenylethynyl)pyridin-2-amine

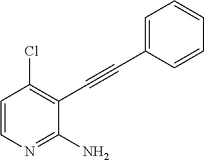 [SMILES: Nc1nccc(Cl)c1C#Cc1ccccc1]

A solution of 4-chloro-3-iodo-pyridine-2-amine (0.265 g, 1.023 mmol), copper (I) iodide (0.0195 g, 0.1023 mmol) and bis(triphenylphosphine) palladium(II) chloride (0.036 g, 0.0512 mmol) in 3 ml of N,N-dimethyformamide-triethylamine (1:4) was degassed in sealed tube followed by addition of ethynylbenzene (0.1255 g, 0.135 mL, 1.23 mmol). The reaction mixture was stirred at 80° C. for 4 h. The reaction mixture was then diluted with EtOAc and extracted with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a light brown solid (0.22 g, 93%). ¹H NMR (500 MHz, DMSO-d₆) δ 7.92 (d, J=5.4 Hz, 1H), 7.70-7.65 (m, 2H), 7.44 (dd, J=5.0, 1.9 Hz, 3H), 6.75 (d, J=5.4 Hz, 1H), 6.68 (s, 2H). LRMS (ESI) m/z [M]⁺. For C₁₃H₉ClN₂ Molecular Weight: 228.6790 found 229.5

### 5-(2-Amino-3-(phenylethynyl)pyridine-4-yl)-1H-indazol-3-amine

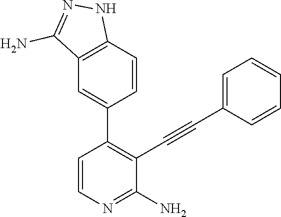 [SMILES: Nc1nccc(-c2ccc3[nH]nc(N)c3c2)c1C#Cc1ccccc1]

4-Chloro-3-(phenylethynyl)pyridin-2-amine (0.22 g, 0.973 mmol), 2-fluoro-5-(4,4,5,5-tetramethyl-1,3,2-dioxaborolan-2-yl)benzonitrile (0.288 g, 1.168 mmol), cesium carbonate (0.951 g, 2.92 mmol), [1,1′-bis(di-tert-butylphosphino)ferrocene]dichloropalladium(II) catalyst (0.032 g, 0.049 mmol, 5 mol %) in 3 mL of a degassed mixture of dioxane and water (18:2 5 mL) was stirred at 110° C. for 18 hours, concentrated and purified by flash chromatography chromatography (50% EtOAc and 1% triethylamine in petroleum ether 60-80%) to afford 5-(2-amino-3-(phenylethynyl)pyridin-4-yl)-2-fluorobenzonitrile.

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder 0.055 g (18%). ¹H NMR (500 MHz, DMSO-d₆) δ 8.31 (d, J=1.6 Hz, 1H), 8.05 (d, J=6.3 Hz, 1H), 7.80 (s, 2H), 7.72 (dd, J=8.7, 1.7 Hz, 1H), 7.59-7.55 (m, 2H), 7.44-7.38 (m, 4H), 7.00 (d, J=6.4 Hz, 1H). LRMS (ESI) m/z [M]⁺. For C₂₀H₁₅N₅ Molecular Weight: 325.3750 found 326.2

### Example 76

### 4-Chloro-3-(4-aminophenyl)ethynyl)pyridin-2-amine

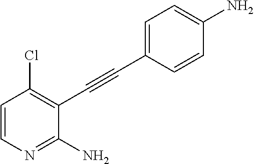 [SMILES: Nc1ccc(C#Cc2c(Cl)ccnc2N)cc1]

A solution of 4-chloro-3-iodo-pyridine-2-amine (0.3 g, 1.18 mmol), copper (1) iodide (0.0225 g, 0.118 mmol) and bis(triphenylphosphine) palladium(II) chloride (0.083 g, 0.118 mmol) in 5 mL of N,N-dimethyformamide-triethylamine (1:4) was degassed in sealed tube followed by addition of 4-ethynylaniline (0.166 g, 0.148 mL, 1.418 mmol). The reaction mixture was stirred at 80° C. for 4 h. The reaction mixture was then diluted with EtOAc and extract with 1M sodium carbonate. Extracted organic layer was washed with brine and dried over magnesium sulfate and concentrated under reduced pressure and the residue purified by column chromatography (50% EtOAc and 1% triethylamine in petroleum ether 60-80%) to give the product as a dark brown oil (0.077 g, 27%)

### 5-(2-Amino-3-((4-aminophenyl)ethynyl)pyridine-4-yl)-1H-indazol-3-amine

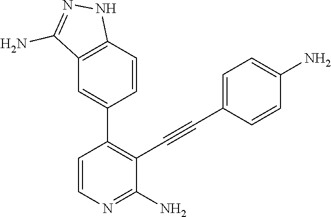 [SMILES: Nc1ccc(C#Cc2c(-c3ccc4[nH]nc(N)c4c3)ccnc2N)cc1]

4-Chloro-3-(4-aminophenyl)ethynyl)pyridin-2-amine (0.077 g, 0.317 mmol), 2-fluoro-5-(4,4,5,5-tetramethyl-1,3,2-dioxaborolan-2-yl)benzonitrile (0.35 g, 1.418 mmol), cesium carbonate (1.35 g, 4.14 mmol), [1,1′-bis(di-tert-butylphosphino)ferrocene]dichloropalladium(II) catalyst (0.077 g, 1.181 mmol) in 3 mL of a degassed mixture of dioxane and water (18:2 5 mL) and stirred at 110° C. for 18 hours, concentrated and purified by flash chromatography to afford 5-(2-amino-3-((4-aminophenyl)ethynyl)pyridin-4-yl)-2-fluorobenzonitrile. ¹H NMR (500 MHz, DMSO-d₆) δ 8.24 (dd, J=6.2, 2.3 Hz, 1H), 8.09 (ddd, J=8.1, 5.3, 2.4 Hz, 1H), 7.98 (d, J=5.2 Hz, 1H), 7.69 (t, J=9.0 Hz, 1H), 7.17-7.13 (m, 2H), 6.70 (d, J=5.1 Hz, 1H), 6.55-6.50 (m, 2H), 6.34 (s, 2H), 5.57 (s, 2H).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder 0.012 g (11%). ¹H NMR (500 MHz, DMSO-d₆) δ 11.50 (s, 1H), 8.08 (d, J=1.6 Hz, 1H), 7.93 (d, J=5.2 Hz, 1H), 7.66 (d, J=8.4 Hz, 1H), 7.60 (dd, J=8.7, 1.7 Hz, 1H), 7.32 (d, J=8.6 Hz, 1H), 7.15 (d, J=8.5 Hz, 3H), 6.69-6.64 (m, 2H), 6.52-6.48 (m, 2H), 6.15 (s, 2H), 5.49 (s, 2H), 5.45 (s, 2H). LRMS (ESI) m/z [M]⁺. For C₂₀H₁₆N₆ Molecular Weight: 340.3900 found 341.3

### Example 77

### 4-Chloro-3-(3-aminophenyl)ethynyl)pyridin-2-amine

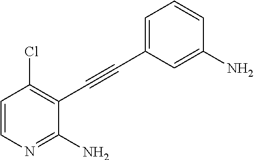 [SMILES: Nc1cccc(C#Cc2c(Cl)ccnc2N)c1]

A solution of 4-chloro-3-iodo-pyridine-2-amine (0.3 g, 1.18 mmol), copper (1) iodide (0.0225 g, 0.118 mmol) and bis(triphenylphosphine) palladium(II) chloride (0.083 g, 0.118 mmol) in 5 mL of N,N-dimethyformamide-triethylamine (1:4) was degassed in sealed tube followed by addition of 3-ethynylaniline (0.166 g, 0.148 mL, 1.418 mmol). The reaction mixture was stirred at 80° C. for 4 h. The reaction mixture was then diluted with EtOAc and extract with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a dark brown oil (0.103 g, 36%)

### 5-(2-Amino-3-((3-aminophenyl)ethynyl)pyridine-4-yl)-1H-indazol-3-amine

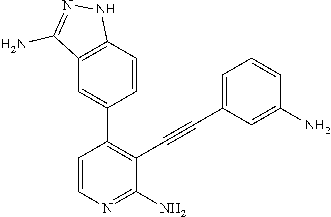 [SMILES: Nc1cccc(C#Cc2c(-c3ccc4[nH]nc(N)c4c3)ccnc2N)c1]

4-Chloro-3-(3-aminophenyl)ethynyl)pyridin-2-amine (0.103 g, 0.424 mmol) 2-fluoro-5-(4,4,5,5-tetramethyl-1,3,2-dioxaborolan-2-yl)benzonitrile (0.35 g, 1.418 mmol) cesium carbonate (1.35 g, 4.14 mmol), [1,1′-bis(di-tert-butylphosphino)ferrocene]dichloropalladium(II) catalyst (0.077 g, 1.181 mmol) in 3 mL of a degassed mixture of dioxane and water (18:2 5 mL) and stirred at 110° C. for 18 hours, concentrated and purified by flash chromatography to afford 5-(2-amino-3-((3-aminophenyl)ethynyl)pyridin-4-yl)-2-fluorobenzonitrile. ¹H NMR (500 MHz, DMSO-d₆) δ 8.28 (dd, J=6.2, 2.3 Hz, 1H), 8.18 (ddd, J=8.9, 5.3, 2.4 Hz, 1H), 8.11 (d, J=5.2 Hz, 1H), 7.77 (t, J=9.0 Hz, 1H), 7.09 (t, J=8.0 Hz, 1H), 6.79 (d, J=5.1 Hz, 1H), 6.70 (dq, J=4.0, 1.3 Hz, 2H), 6.65 (ddd, J=8.0, 2.4, 1.1 Hz, 1H), 6.50 (s, 2H), 5.25 (s, 2H).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder 0.010 g (10%). ¹H NMR (500 MHz, DMSO-d₆) δ 11.59 (s, 1H), 8.14-8.10 (m, 1H), 8.03 (d, J=5.3 Hz, 1H), 7.66 (dd, J=8.7, 1.7 Hz, 1H), 7.41-7.35 (m, 1H), 7.04 (t, J=7.8 Hz, 1H), 6.76-6.66 (m, 3H), 6.60 (ddd, J=8.1, 2.4, 1.1 Hz, 1H), 5.53 (s, 2H), 5.17 (s, 2H). LRMS (ESI) m/z [M]⁺. For C₂₀H₁₆N₆ Molecular Weight: 340.3900 found 341.3

### Example 78

### 4-Chloro-3-(2-aminophenyl)ethynyl)pyridin-2-amine

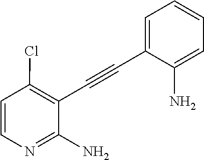 [SMILES: Nc1ccccc1C#Cc1c(Cl)ccnc1N]

A solution of 4-chloro-3-iodo-pyridine-2-amine (0.3 g, 1.181 mmol), copper (1) iodide (0.023 g. 0.1181 mmol), and bis(triphenylphosphine) palladium(II) chloride (0.083 g, 0.1181 mmol) in 5 ml of N,N-dimethyformamide-triethylamine (1:4) was degassed in sealed tube followed by addition of 2-ethynylaniline (0.166 g, 1.42 mmol). The reaction mixture was stirred at 80° C. for 4 h. The reaction mixture was then diluted with EtOAc and extract with 1M sodium carbonate. Extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a dark brown oil (0.093 g, 32%)

### 5-(2-Amino-3-((2-aminophenyl)ethynyl)pyridine-4-yl)-1H-indazol-3-amine

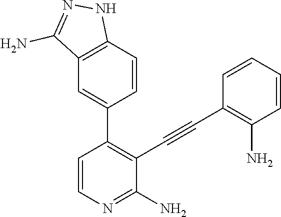 [SMILES: Nc1ccccc1C#Cc1c(-c2ccc3[nH]nc(N)c3c2)ccnc1N]

4-Chloro-3-(2-aminophenyl)ethynyl)pyridin-2-amine (0.093 g, 0.382 mmol), 2-fluoro-5-(4,4,5,5-tetramethyl-1,3,2-dioxaborolan-2-yl)benzonitrile (0.113 g, 0.459 mmol), cesium carbonate (0.374 g, 1.147 mmol), [1,1′-bis(di-tert-butylphosphino)ferrocene]dichloropalladium(II) catalyst (0.025 g, 0.038 mmol) in 5 mL of a degassed mixture of dioxane and water (18:2 5 mL) and stirred at 110° C. for 18 hours, concentrated and purified by flash chromatography to afford 5-(2-amino-3-((2-aminophenyl)ethynyl)pyridin-4-yl)-2-fluorobenzonitrile.

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.008 g, 6%). ¹H NMR (500 MHz, DMSO-d₆) δ 11.53 (s, 1H), 8.07 (d, J=1.5 Hz, 1H), 7.99 (d, J=5.2 Hz, 1H), 7.61 (dd, J=8.7, 1.6 Hz, 1H), 7.33 (d, J=8.6 Hz, 1H), 6.99 (t, J=7.8 Hz, 1H), 6.68 (d, J=5.2 Hz, 2H), 6.64 (d, J=7.5 Hz, 1H), 6.55 (dd, J=8.0, 2.2 Hz, 1H), 6.23 (s, 2H), 5.48 (s, 2H), 5.12 (s, 2H). LRMS (ESI) m/z [M]⁺. For C₂₀H₁₆N₆ Molecular Weight: 340.3900 found 341.3

### Example 79

### Methyl 3-((2-amino-4-chloropyridin-3-yl)ethynyl)benzoate

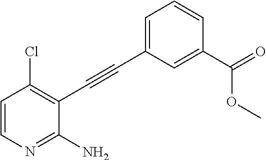 [SMILES: COC(=O)c1cccc(C#Cc2c(Cl)ccnc2N)c1]

A solution of 4-chloro-3-iodo-pyridine-2-amine (0.3 g, 1.181 mmol), copper (1) iodide (0.023 g. 0.1181 mmol), and bis(triphenylphosphine) palladium(II) chloride (0.083 g, 0.1181 mmol) in 5 ml of N,N-dimethyformamide-triethylamine (1:4) was degassed in sealed tube followed by addition of methyl 3-ethynylbenzoate (0.227 g, 1.417 mmol). The reaction mixture was stirred at 80° C. for 4 h. The reaction mixture was then diluted with EtOAc and extract with 1M sodium carbonate. Extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a light brown solid (0.062 g, 18%)

### Methyl 3-((2-amino-4-(3-amino-1H-indazol-5-yl)149yridine-3-yl)ethynyl)benzoate

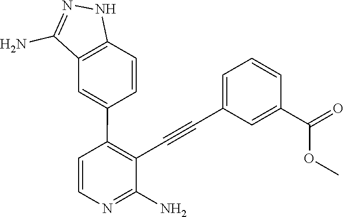 [SMILES: COC(=O)c1cccc(C#Cc2c(-c3ccc4[nH]nc(N)c4c3)ccnc2N)c1]

Methyl 3-((2-amino-4-chloropyridin-3-yl)ethynyl)benzoate (0.062 g, 0.216 mmol), 2-fluoro-5-(4,4,5,5-tetramethyl-1,3,2-dioxaborolan-2-yl)benzonitrile (0.064 g, 0.259 mmol), cesium carbonate (0.211 g, 0.648 mmol), [1,1′-bis(di-tert-butylphosphino)ferrocene]dichloropalladium(II) catalyst (0.014 g, 0.0216 mmol) in 5 mL of a degassed mixture of dioxane and water (18:2 5 mL) and stirred at 110° C. for 18 hours, concentrated and purified by flash chromatography (50% EtOAc and 1% triethylamine in petroleum ether 60-80%) to afford methyl 3-((2-amino-4-(3-cyano-4-fluorophenyl)pyridin-3-yl)ethynyl)benzoate. ¹H NMR (500 MHz, DMSO-d₆) δ 8.26 (dd, J=6.3, 2.4 Hz, 1H), 8.13-8.05 (m, 3H), 7.94 (dt, J=7.9, 1.5 Hz, 1H), 7.73-7.66 (m, 2H), 7.55 (t, J=7.8 Hz, 1H), 6.74-6.66 (m, 3H), 3.89 (s, 3H).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.017 g, 6%). ¹H NMR (500 MHz, DMSO-d₆) δ 12.19 (s, 1H), 11.64 (s, 1H), 8.53 (d, J=5.0 Hz, 1H), 7.90-7.85 (m, 2H), 7.74-7.66 (m, 1H), 7.42-7.34 (m, 2H), 7.22 (d, J=5.0 Hz, 1H), 6.57 (d, J=9.8 Hz, 1H), 5.51 (s, 2H). (CH₃ Hidden under 3.3H₂O signal). LRMS (ESI) m/z [M]⁺. For C₂₂H₁₇N₅O₂ Molecular Weight: 383.4110 found 384.3

### Example 80

### Methyl 4-((2-amino-4-chloropyridin-3-yl)ethynyl)benzoate

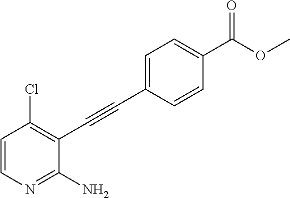 [SMILES: COC(=O)c1ccc(C#Cc2c(Cl)ccnc2N)cc1]

A solution of 4-chloro-3-iodo-pyridine-2-amine (0.3 g, 1.181 mmol), copper (1) iodide (0.023 g. 0.1181 mmol), and bis(triphenylphosphine) palladium(II) chloride (0.083 g, 0.1181 mmol) in 5 ml of N,N-dimethyformamide-triethylamine (1:4) was degassed in sealed tube followed by addition of methyl 4-ethynylbenzoate (0.227 g, 1.417 mmol). The reaction mixture was stirred at 80° C. for 4 h. The reaction mixture was then diluted with EtOAc and extract with 1M sodium carbonate. Extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown solid. (0.169 g, 50%).

### Methyl 4-((2-amino-4-(3-amino-1H-indazol-5-yl)150yridine-3-yl)ethynyl)benzoate

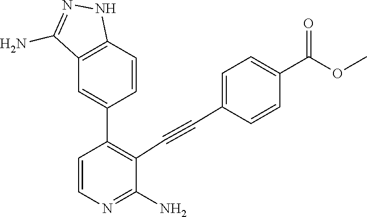 [SMILES: COC(=O)c1ccc(C#Cc2c(-c3ccc4[nH]nc(N)c4c3)ccnc2N)cc1]

Methyl 3-((2-amino-4-chloropyridin-3-yl)ethynyl)benzoate (0.169 g, 0.589 mmol), 2-fluoro-5-(4,4,5,5-tetramethyl-1,3,2-dioxaborolan-2-yl)benzonitrile (0.175 g, 0.707 mmol), cesium carbonate (0.576 g, 1.767 mmol), [1,1′-bis(di-tert-butylphosphino)ferrocene]dichloropalladium(II) catalyst (0.038 g, 0.0589 mmol) in 5 mL of a degassed mixture of dioxane and water (18:2 5 mL) and stirred at 110° C. for 18 hours, concentrated and purified by flash chromatography (50% EtOAc and 1% triethylamine in petroleum ether 60-80%) to afford methyl 4-((2-amino-4-(3-cyano-4-fluorophenyl)pyridin-3-yl)ethynyl)benzoate.

The obtained methyl 4-((2-amino-4-(3-cyano-4-fluorophenyl)pyridin-3-yl)ethynyl)benzoate was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.017 g, 6%). ¹H NMR (500 MHz, DMSO-d₆) δ 11.55 (s, 1H), 9.81 (s, 1H), 8.13 (s, 1H), 8.02 (d, J=5.2 Hz, 1H), 7.79 (d, J=8.0 Hz, 1H), 7.57 (dd, J=20.2, 8.3 Hz, 2H), 7.35 (d, J=8.7 Hz, 1H), 6.70 (d, J=5.2 Hz, 1H), 6.43 (s, 2H), 5.50 (s, 2H), 3.88 (s, 3H). LRMS (ESI) m/z [M]⁺. For C₂₂H₁₇N₅O₂ Molecular Weight: 383.411 found 384.3

### Example 814-Chloro-3-(2-methoxyphenyl)ethynyl)pyridin-2-amine

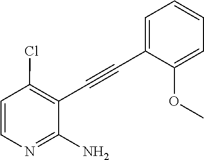 [SMILES: COc1ccccc1C#Cc1c(Cl)ccnc1N]

A solution of 4-chloro-3-iodo-pyridine-2-amine (0.3 g, 1.181 mmol), copper (1) iodide (0.023 g, 0.1181 mmol) and bis(triphenylphosphine) palladium(II) chloride (0.083 g, 0.1181 mmol) in 5 ml of N,N-dimethyformamide-triethylamine (1:4) was degassed in sealed tube followed by addition of 1-ethynyl-2-methoxybenzene (0.187 g 1.417 mmol). The reaction mixture was stirred at 80° C. for 4 h. The reaction mixture was then diluted with EtOAc and extract with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a dark brown solid (0.215 g, 70%).

### 5-(2-Amino-3-((2-methoxyphenyl)ethynyl)pyridine-4-yl)-1H-indazol-3-amine

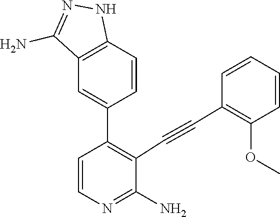 [SMILES: COc1ccccc1C#Cc1c(-c2ccc3[nH]nc(N)c3c2)ccnc1N]

4-Chloro-3-(2-methoxyphenyl)ethynyl)pyridin-2-amine (0.215 g, 0.831 mmol), 2-fluoro-5-(4,4,5,5-tetramethyl-1,3,2-dioxaborolan-2-yl)benzonitrile (0.247 g, 0.997 mmol) cesium carbonate (0.814 g, 2.5 mmol), [1,1′-bis(di-tert-butylphosphino)ferrocene]dichloropalladium(II) catalyst (0.0543 g, 0.08327 mmol) in 5 mL of a degassed mixture of dioxane and water (18:2 5 mL) and stirred at 110° C. for 18 hours, concentrated and purified by flash chromatography (50% EtOAc and 1% triethylamine in petroleum ether 60-80%) to afford 5-(2-amino-3-((2-methoxyphenyl)ethynyl)pyridin-4-yl)-2-fluorobenzonitrile.

The obtained fluorobenzonitrile was then dissolved in ethanol (8 ml) and hydrazine, (2 mL, 1 Mol solution in Ethanol) added. The resulting mixture was stirred at reflux for 2 hours, cooled concentrated and purified by HPLC affording the target compound as a yellow powder (0.045 g, 15%). ¹H NMR (500 MHz, DMSO-d₆) δ 8.26 (d, J=1.6 Hz, 1H), 8.06 (d, J=6.2 Hz, 1H), 7.78 (dd, J=8.8, 1.7 Hz, 1H), 7.49 (dd, J=7.6, 1.7 Hz, 1H), 7.44-7.37 (m, 3H), 7.10 (d, J=8.4 Hz, 1H), 6.98 (t, J=7.0 Hz, 2H), 3.80 (s, 3H). LRMS (ESI) m/z [M]⁺. For C₂₁H₁₇N₅O Molecular Weight: 355.4010 found 356.3

### Example 82

### 5-(2-Amino-4-(3-amino-1H-indazol-5-yl)pyridine-3-yl)-1-phenylpent-4-yn-1-one

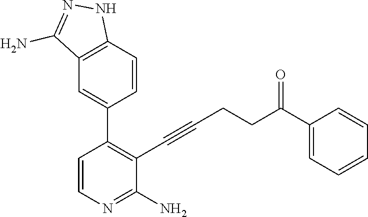 [SMILES: Nc1nccc(-c2ccc3[nH]nc(N)c3c2)c1C#CCCC(=O)c1ccccc1]

A solution of 5-(2-amino-3-bromopyridin-4-yl)-1H-indazol-3-amine (0.12 g, 0.48 mmol), copper (1) iodide (0.004 g, 0.024 mmol) and bis(triphenylphosphine) palladium(II) chloride (0.016 g, 0.024 mmol) in 3 ml of N,N-dimethyformamide-triethylamine (1:4) was degassed in sealed tube followed by addition of 1-phenylpent-4-yn-1-one (0.13 g, 0.58 mmol). The reaction mixture was stirred at 80° C. for 4 h. The reaction mixture was then diluted with EtOAc and extracted with 1 M sodium carbonate. The extracted organic layer was washed with brine and dried over magnesium sulfate and concentrated under reduced pressure and the residue purified by column chromatography (20% EtOAc in petroleum ether 60-80%) to give the product as yellow off white solid (0.13 g, 77%), ¹H NMR (400 MHz, DMSO-d₆) δ 2.75 (t, J=12.3 Hz, 2H), 2.75 (t, J=12.3 Hz, 2H), 5.41 (br. s., 2H) 6.25 (br. s., 2H), 6.63 (d, J=5.71 Hz, 1H), 7.28 (m, 2H), 7.75 (s, 1H), 7.95 (m, 2H), 11.55 (s, 1H), LRMS: C₂₃H₁₉N₅O requires 381.2, found 382.1 (M+H).

### Example 83

### 3-(2-Amino-4-chloropyridin-3-yl)prop-2-yn-1-ol

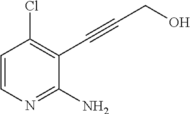 [SMILES: Nc1nccc(Cl)c1C#CCO]

A solution of 4-chloro-3-iodo-pyridine-2-amine (0.3 g, 1.181 mmol), copper (1) iodide (0.023 g, 0.1181 mmol) and bis(triphenylphosphine) palladium(II) chloride (0.083 g, 0.1181 mmol) in 5 ml of N,N-dimethyformamide-triethylamine (1:4) was degassed in sealed tube followed by addition of propagyl alcohol (0.0795 g, 0.083 mL, 1.42 mmol). The reaction mixture was stirred at 80° C. for 4 h. The reaction mixture was then diluted with EtOAc and extracted with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a dark brown solid (0.087 g, 40%). ¹H NMR (500 MHz, DMSO-d₆) δ 7.87 (d, J=5.4 Hz, 1H), 6.68 (d, J=5.5 Hz, 1H), 6.56 (s, 2H), 5.35 (t, J=6.0 Hz, 1H), 4.38 (d, J=6.0 Hz, 2H). LRMS (ESI) m/z [M]⁺. For C₈H₇ClN₂O Molecular Weight: 182.6070 found 183.1

### 3-(2-Amino-4-(3-amino-1H-indazol-5-yl)pyridine-3-yl)prop-2-yn-1-ol

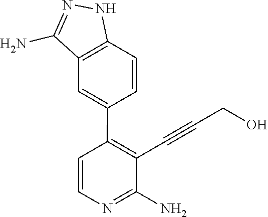 [SMILES: Nc1nccc(-c2ccc3[nH]nc(N)c3c2)c1C#CCO]

3-(2-Amino-4-chloropyridin-3-yl)prop-2-yn-1-ol (0.087 g, 0.476 mmol), 2-fluoro-5-(4,4,5,5-tetramethyl-1,3,2-dioxaborolan-2-yl)benzonitrile (0.141 g, 0.572 mmol) cesium carbonate (0.465 g, 1.428 mmol), [1,1′-bis(di-tert-butylphosphino)ferrocene]dichloropalladium(II) catalyst (0.031 g, 0.0476 mmol) in 5 mL of a degassed mixture of dioxane and water (18:2 5 mL) and stirred at 110° C. for 18 hours, concentrated and purified by flash chromatography (50% EtOAc and 1% triethylamine in petroleum ether 60-80%) to afford 5-(2-amino-3-(3-hydroxyprop-1-yn-1-yl)pyridin-4-yl)-2-fluorobenzonitrile.

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a cream powder (0.060 g, 45%). ¹H NMR (500 MHz, DMSO-d₆) δ 8.21 (d, J=1.6 Hz, 1H), 8.01 (d, J=6.5 Hz, 1H), 7.80 (s, 2H), 7.69 (dd, J=8.8, 1.8 Hz, 1H), 7.36 (d, J=8.8 Hz, 1H), 6.98 (d, J=6.5 Hz, 1H), 4.31 (s, 2H). LRMS (ESI) m/z [M]⁺. For C₁₅H₁₃N₅O Molecular Weight: 279.3030 found 280.1

### Example 84

### 3-(2-Amino-4-chloropyridin-3-yl) but-3-yn-1-ol

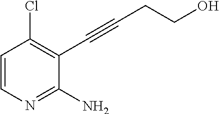 [SMILES: Nc1nccc(Cl)c1C#CCCO]

A solution of 4-chloro-3-iodo-pyridine-2-amine (0.3 g, 1.181 mmol), copper (I) iodide (0.023 g, 0.1181 mmol) and bis(triphenylphosphine) palladium(II) chloride (0.083 g, 0.1181 mmol) in 5 ml of N,N-dimethyformamide-triethylamine (1:4) was degassed in sealed tube followed by addition of 3-butyn-1-ol (0.0993 g, 0.107 mL, 1.42 mmol). The reaction mixture was stirred at 80° C. for 4 h. The reaction mixture was then diluted with EtOAc and extracted with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown solid (0.184 g, 79%). ¹H NMR (500 MHz, DMSO-d₆) δ 7.91 (s, 1H), 6.73 (d, J=5.3 Hz, 1H), 6.54 (s, 2H), 5.05 (t, J=5.6 Hz, 1H), 3.68 (td, J=6.6, 5.4 Hz, 2H), 2.71 (t, J=6.6 Hz, 2H). LRMS (ESI) m/z [M]⁺. For C₉H₉ClN₂O Molecular Weight: 196.6340 found 197.1

### 4-(2-Amino-4-(3-amino-1H-indazol-5-yl)pyridine-3-yl)but-3-yn-1-ol

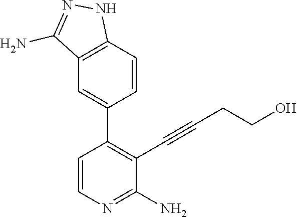 [SMILES: Nc1nccc(-c2ccc3[nH]nc(N)c3c2)c1C#CCCO]

3-(2-Amino-4-chloropyridin-3-yl)but-3-yn-1-ol (0.184 g, 0.93 mmol), 2-fluoro-5-(4,4,5,5-tetramethyl-1,3,2-dioxaborolan-2-yl)benzonitrile (0.278 g, 1.12 mmol), cesium carbonate (0.303 g, 2.79 mmol), [1,1′-bis(di-tert-butylphosphino)ferrocene]dichloropalladium(II) catalyst (0.0606 g, 0.093 mmol) in 5 mL of a degassed mixture of dioxane and water (18:2 5 mL) and stirred at 110° C. for 18 hours, concentrated and purified by flash chromatography (50% EtOAc and 1% triethylamine in petroleum ether 60-80%) to afford 5-(2-amino-3-(4-hydroxybut-1-yn-1-yl)pyridin-4-yl)-2-fluorobenzonitrile. ¹H NMR (500 MHz, DMSO-d₆) δ 8.16 (dd, J=6.2, 2.4 Hz, 1H), 8.05 (ddd, J=8.8, 5.3, 2.4 Hz, 1H), 7.97 (d, J=5.2 Hz, 1H), 7.62 (t, J=9.0 Hz, 1H), 6.64 (d, J=5.2 Hz, 1H), 6.36 (s, 2H), 4.93 (t, J=5.6 Hz, 1H), 3.53 (td, J=6.7, 5.5 Hz, 2H), 2.54 (d, J=6.7 Hz, 2H).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a cream powder (0.059 g, 22%). ¹H NMR (400 MHz, DMSO-d₆) δ 8.26 (d, J=1.7 Hz, 1H), 7.70 (dd, J=8.6, 1.9 Hz, 1H), 7.36 (d, J=8.8 Hz, 1H), 6.99 (d, J=6.6 Hz, 1H), 2.59 (t, J=6.6 Hz, 2H). LRMS (ESI) m/z [M]⁺. For C₁₆H₁₅N₅O Molecular Weight: 293.3300 found 294.1

### Example 85

### 3-(2-Amino-4-chloropyridin-3-yl)pent-4-yn-1-ol

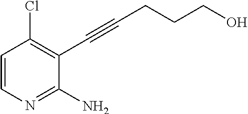 [SMILES: Nc1nccc(Cl)c1C#CCCCO]

A solution of 4-chloro-3-iodo-pyridine-2-amine (0.3 g/1.18 mmol), copper (I) iodide (0.0225 g, 0.118 mmol) and bis(triphenylphosphine) palladium(II) chloride (0.0829 g, 0.118 mmol) in 5 ml of N,N-dimethyformamide-triethylamine (1:4) was degassed in sealed tube followed by addition of 4-pentyn-1-ol (0.119 g, 0.132 mL, 1.417 mmol). The reaction mixture was stirred at 80° C. for 4 h. The reaction mixture was then diluted with EtOAc and washed with 1M sodium carbonate.

The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown solid (0.192 g, 77%). ¹H NMR (500 MHz, DMSO-d₆) δ 7.84 (s, 1H), 6.67 (d, J=5.4 Hz, 1H), 6.44 (s, 2H), 4.59 (t, J=5.2 Hz, 1H), 3.54 (td, J=6.1, 5.1 Hz, 2H), 2.56 (t, J=7.0 Hz, 2H), 1.76-1.67 (m, 2H). LRMS (ESI) m/z [M]⁺. For C₁₀H₁₁ClN₂O Molecular weight 210.6610 found 211.5.

### 5-(2-Amino-4-(3-amino-1H-indazol-5-yl)pyridine-3-yl)pent-4-yn-1-ol

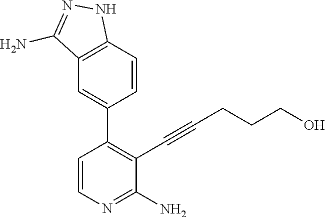 [SMILES: Nc1nccc(-c2ccc3[nH]nc(N)c3c2)c1C#CCCCO]

3-(2-Amino-4-chloropyridin-3-yl)pent-4-yn-1-ol (0.192 g, 0.914 mmol), 2-fluoro-5-(4,4,5,5-tetramethyl-1,3,2-dioxaborolan-2-yl)benzonitrile (0.2709 g, 1.096 mmol), [1,1′-bis(di-tert-butylphosphino)ferrocene]dichloropalladium(II) catalyst (0.0297 g, 0.0457 mmol, 5 mol %) and cesium carbonate (0.952 g, 2.92 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-(5-hydroxypent-1-yn-1-yl)pyridin-4-yl)-2-fluorobenzonitrile (0.117 g, 43%).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.090 g, 32%). ¹H NMR (500 MHz, DMSO-d₆) δ 8.14 (dd, J=6.3, 2.3 Hz, 1H), 8.02 (ddd, J=8.8, 5.3, 2.4 Hz, 1H), 7.96 (d, J=5.2 Hz, 1H), 7.63 (t, J=9.1 Hz, 1H), 6.64 (d, J=5.2 Hz, 1H), 6.31 (s, 2H), 4.51 (t, J=5.2 Hz, 1H), 3.40 (q, J=5.9 Hz, 2H), 2.44 (t, J=7.0 Hz, 2H), 1.66-1.55 (m, 2H). LRMS (ESI) m/z [M]⁺. For C₁₇H₁₇N₅O Molecular Weight: 307.3570 found 308.1

### Example 86

### 6-(2-Amino-4-(3-amino-1H-indazol-5-yl)pyridine-3-yl)-2-methylhex-5-yn-2-ol

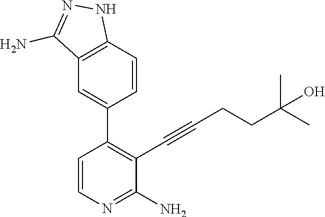 [SMILES: CC(C)(O)CCC#Cc1c(-c2ccc3[nH]nc(N)c3c2)ccnc1N]

A 2-5 mL Biotage MW tube was charged with 6-(2-amino-4-chloropyridin-3-yl)-2-methylhex-5-yn-2-ol (100 mg, 0.3 mmol), 5-(4,4,5,5-tetramethyl-1,3,2-dioxaborolan-2-yl)-1H-indazol-3-amine (103 mg, 0.4 mmol, 1.3 eq.), [1,1′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃PO₄ (1.8 mL). The reaction mixture was heated to 110° C. for 16 hrs. The reaction mixture was then allowed to cool to room temperature, before being diluted with water (10 mL) and extracted with ethyl acetate (3×20 mL). The combined organic fractions were then dried over anhydrous magnesium sulfate, filtered and the solvent removed under reduced pressure. The crude product was then purified by HPLC to give the desired product as a pale yellow solid (23 mg). ¹H NMR (500 MHz, DMSO-D₆) δ 1.54 (s, 6H), 2.28 (t, J=6.9 Hz, 1H), 2.56 (t, J=7.0 Hz, 1H), 6.72 (d, J=6.6 Hz, 1H), 7.27 (d, J=8.7 Hz, 1H), 7.68 (dd, J=8.4 and 1.6 Hz, 1H), 7.89 (d, J=8.0 Hz, 1H), 8.24 (s, 1H), 11.48 (s, 1H). LRMS: Calculated for C₁₉H₂₁N₅O 335.17; Found: 336.25

### Example 87

### 3-(2-Amino-4-chloropyridin-3-yl)-2-methylbut-3-yn-2-ol

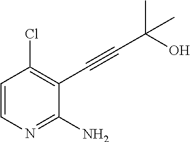

A solution of 4-chloro-3-iodo-pyridine-2-amine (0.26 g, 1.02 mmol), copper (1) iodide (0.0097 g, 0.051 mmol) and bis(triphenylphosphine) palladium(II) chloride (0.0358 g, 0.051 mmol) in 3 ml of N,N-dimethyformamide-triethylamine (1:4) was degassed in sealed tube followed by addition of 2-methyl-3-butyn-2-ol (0.103 g, 0.119 mL, 1.228 mmol). The reaction mixture was stirred at 80° C. for 4 h. The reaction mixture was then diluted with EtOAc and washed with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a light brown solid. (0.177 g, 84%). ¹H NMR (500 MHz, DMSO-d₆) δ 7.93 (d, J=23.9 Hz, 1H), 6.70 (d, J=4.7 Hz, 1H), 6.48 (s, 2H), 5.59 (s, 1H), 1.49 (s, 6H). LRMS (ESI) m/z [M]⁺. C₁₀H₁₁ClN₂O Molecular Weight: 210.6610 found 221.5

### 4-(2-Amino-4-(3-amino-1H-indazol-5-yl)pyridine-3-yl)-2-methylbut-3-yn-2-ol

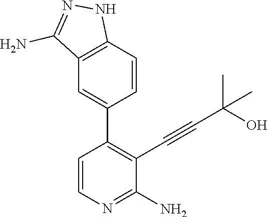 [SMILES: CC(C)(O)C#Cc1c(-c2ccc3[nH]nc(N)c3c2)ccnc1N]

3-(2-Amino-4-chloropyridin-3-yl)-2-methylbut-3-yn-2-ol (0.244 g, 1.161 mmol), 2-fluoro-5-(4,4,5,5-tetramethyl-1,3,2-dioxaborolan-2-yl)benzonitrile (0.344 g, 1.39 mmol), [1,1′-bis(di-tert-butylphosphino)ferrocene]dichloropalladium(II) catalyst (0.038 g, 0.058 mmol, 5 mol %), cesium carbonate (0.952 g, 2.92 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-(3-hydroxy-3-methylbut-1-yn-1-yl)pyridin-4-yl)-2-fluorobenzonitrile (0.231 g, 67%).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.054 g, 15%). ¹H NMR (500 MHz, DMSO-d₆) δ 8.25 (d, J=1.6 Hz, 1H), 8.02 (d, J=6.5 Hz, 1H), 7.68 (dd, J=8.8, 1.8 Hz, 1H), 7.36 (d, J=8.8 Hz, 1H), 7.00 (d, J=6.5 Hz, 1H), 1.40 (s, 6H). LRMS (ESI) m/z [M]⁺. C₁₇H₁₇N₅O Molecular Weight: 307.3570 found 308.1

### Example 88

### 3-(3-(Tert-butoxy)prop-1-yn-1-yl)-4-chloropyridin-2-amine

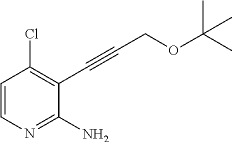 [SMILES: CC(C)(C)OCC#Cc1c(Cl)ccnc1N]

A solution of 4-chloro-3-iodo-pyridine-2-amine (0.25 g, 0.98 mmol), copper (I) iodide (0.01 g, 0.05 mmol) and bis(triphenylphosphine) palladium(II) chloride (0.03 g, 0.05 mmol) in 3 ml of N,N-dimethyformamide-triethylamine (1:4) was degassed in sealed tube followed by addition of 3-(tert-butoxy)prop-1-yne (0.2 ml, 1.4 mmol). The reaction mixture was stirred at 80° C. for 4 h. The reaction mixture was then diluted with EtOAc and extract with 1M sodium carbonate. Extracted organic layer was washed with brine and dried over magnesium sulfate and concentrated under reduced pressure and the residue purified by column chromatography (50% EtOAc and 1% triethylamine in petroleum ether 60-80%) to give the product as yellow oil (0.116 g, 49%). ¹H NMR (400 MHz, DMSO-d₆) δ ppm 1.22 (s, 9H) 4.39 (s, 2H) 6.51 (br. s., 2H) 6.69 (d, J=5.49 Hz, 1H) 7.87 (d, J=5.34 Hz, 1H). ¹³C NMR (100 MHz, DMSO-d₆) b ppm 27.91, 40.28, 51.31, 64.79, 77.20, 99.49, 112.76, 115.07, 148.83, 161.65. m/z (ESI-MS) [M]⁺ 239.0.

### 5-(2-Amino-3-(3-(tert-butoxy)prop-1-yn-1-yl)pyridine-4-yl)-1H-indazol-3-amine

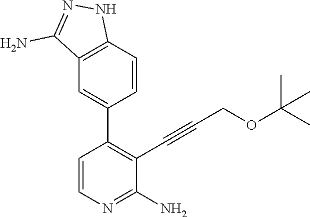 [SMILES: CC(C)(C)OCC#Cc1c(-c2ccc3[nH]nc(N)c3c2)ccnc1N]

3-(3-(Tert-butoxy)prop-1-yn-1-yl)-4-chloropyridin-2-amine (0.028 g, 0.11 mmol), 5-(4,4,5,5-tetramethyl-1,3,2-dioxaborolan-2-yl)-1H-indazol-3-amine (0.045 g, 0.17 mmol), 1M potassium phosphate solution (0.23 ml, 0.23 mmol), [1,1′-bis(di-tert-butylphosphino)ferrocene]dichloropalladium(II) catalyst₂ (0.004 g, 0.005 mmol) in 0.7 ml ethanol were reacted as described in general procedure and chromatographic purification (90% EtOAc and 1% triethylamine in petroleum ether 60-80%), gave the titled compound as brown solid (12.3 mg, 33%). ¹H NMR (400 MHz, DMSO-d₆) δ ppm 1.11 (s, 9H) 4.27 (s, 2H) 5.41 (s, 2H) 6.17 (br. s., 2H) 6.62 (d, J=5.19 Hz, 1H) 7.25 (d, J=8.70 Hz, 1H) 7.53 (dd, J=8.70, 1.53 Hz, 1H) 7.95 (m, 2H) 11.49 (s, 1H). ¹³C NMR (100 MHz, DMSO-d₆) δ ppm 27.83, 40.27, 51.27, 80.18, 97.07, 99.45, 109.24, 113.28, 121.09, 127.43, 128.32, 134.19, 141.55, 144.83, 147.75, 150.25, 152.16. m/z (ESI-HRMS) calculated for C₁₉H₂₂ON₅=336.1819 found=336.1816.

### Example 89

### 1-((2-Amino-4-chloropyridin-3-yl)ethynyl)cyclopentan-1-ol

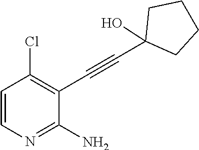 [SMILES: Nc1nccc(Cl)c1C#CC1(O)CCCC1]

A solution of 4-chloro-3-iodo-pyridine-2-amine (0.105, 0.416 mmol), copper (I) iodide (0.0079 g, 0.0416 mmol) and bis(triphenylphosphine) palladium(II) chloride (0.029 g, 0.0416 mmol) in 5 ml of N,N-dimethyformamide-triethylamine (1:4) was degassed in sealed tube followed by addition of 1-ethynylcyclopentan-1-ol (0.055 g, 0.49 mmol). The reaction mixture was stirred at 80° C. for 4 h. The reaction mixture was then diluted with EtOAc and washed with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a brown solid (0.088 g, 90%). ¹H NMR (500 MHz, DMSO-d₆) δ 7.86 (d, J=5.4 Hz, 1H), 6.69 (d, J=5.4 Hz, 1H), 6.50-6.32 (m, 2H), 5.45 (s, 1H), 1.92 (q, J=5.6, 4.6 Hz, 4H), 1.81-1.62 (m, 4H). LRMS (ESI) m/z [M]⁺. C₁₂H₁₃ClN₂O Molecular Weight: 236.6990 found 237.1

### 1-((2-Amino-4-(3-amino-1H-indazol-5-yl)pyridine-3-yl)ethynyl)cyclopentan-1-ol

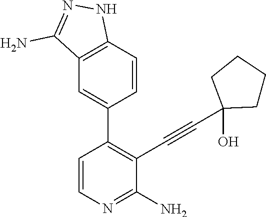 [SMILES: Nc1nccc(-c2ccc3[nH]nc(N)c3c2)c1C#CC1(O)CCCC1]

1-((2-Amino-4-chloropyridin-3-yl)ethynyl)cyclopentan-1-ol (0.088 g, 0.372 mmol), 2-fluoro-5-(4,4,5,5-tetramethyl-1,3,2-dioxaborolan-2-yl)benzonitrile (0.1102 g, 0.44 mmol), [1,1′-bis(di-tert-butylphosphino)ferrocene]dichloropalladium(II) catalyst (0.0121 g, 0.018 mmol, 5 mol %), cesium carbonate (0.363 g, 1.115 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-((1-hydroxycyclopentyl)ethynyl)pyridin-4-yl)-2-fluorobenzonitrile.

The obtained fluorobenzonitrile was then dissolved in ethanol (8 ml) and hydrazine, (2 mL, 1 Mol solution in Ethanol) added. The resulting mixture was stirred at reflux for 2 hours, cooled concentrated and purified by HPLC affording the target compound as a yellow powder (0.022 g, 18%). ¹H NMR (500 MHz, DMSO-d₆) δ 8.12 (d, J=2.3 Hz, 1H), 8.03 (d, J=6.1 Hz, 1H), 7.97 (dd, J=8.9, 2.4 Hz, 1H), 7.38 (d, J=9.0 Hz, 1H), 6.94 (d, J=6.1 Hz, 1H), 1.88-1.83 (m, 4H), 1.75-1.70 (m, 2H), 1.61 (tq, J=7.1, 3.8, 2.7 Hz, 2H). LRMS (ESI) m/z [M]⁺. C₁₅H₁₉N₅O Molecular Weight: 333.3950 found 334.3

### Example 90

### 1-((2-Amino-4-chloropyridin-3-yl)ethynyl)cyclohexan-1-ol

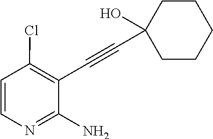 [SMILES: Nc1nccc(Cl)c1C#CC1(O)CCCCC1]

A solution of 4-chloro-3-iodo-pyridine-2-amine (0.4 g, 1.575 mmol), copper (I) iodide (0.015 g, 0.079 mmol) and bis(triphenylphosphine) palladium(II) chloride (0.055 g, 0.079 mmol) in 5 ml of N,N-dimethyformamide-triethylamine (1:4) was degassed in sealed tube followed by addition of 1-ethynylcyclohexan-1-ol (0.234 g, 1.89 mmol). The reaction mixture was stirred at 80° C. for 4 h. The reaction mixture was then diluted with EtOAc and extract with 1M sodium carbonate.

The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a light brown solid (0.282 g, 71%). ¹H NMR (500 MHz, DMSO-d₆) δ 7.86 (d, J=5.5 Hz, 1H), 6.69 (d, J=5.5 Hz, 1H), 6.46 (s, 2H), 5.61 (s, 1H), 1.88 (dd, J=9.2, 5.4 Hz, 2H), 1.74-1.60 (m, 2H), 1.53 (d, J=8.6 Hz, 5H), 1.30-1.13 (m, 1H). LRMS (ESI) m/z [M]⁺.

C₁₃H₁₅ClN₂O Molecular Weight: 250.7260 found 251.1

### 1-((2-Amino-4-(3-amino-1H-indazol-5-yl)pyridine-3-yl)ethynyl)cyclohexan-1-ol

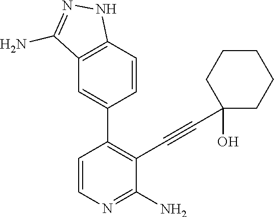 [SMILES: Nc1nccc(-c2ccc3[nH]nc(N)c3c2)c1C#CC1(O)CCCCC1]

1-((2-Amino-4-chloropyridin-3-yl)ethynyl)cyclohexan-1-ol (0.088 g, 0.372 mmol), 2-fluoro-5-(4,4,5,5-tetramethyl-1,3,2-dioxaborolan-2-yl)benzonitrile (0.1102 g, 0.44 mmol), [1,1′-bis(di-tert-butylphosphino)ferrocene]dichloropalladium(II) catalyst (0.0121 g, 0.018 mmol, 5 mol %), cesium carbonate (0.363 g, 1.115 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-((1-hydroxycyclohexyl)ethynyl)pyridin-4-yl)-2-fluorobenzonitrile (0.332 g, 88%). ¹H NMR (500 MHz, DMSO-d₆) δ 8.16 (dd, J=6.3, 2.3 Hz, 1H), 8.00 (d, J=5.2 Hz, 1H), 7.97 (ddd, J=8.7, 5.3, 2.3 Hz, 1H), 7.64 (t, J=9.1 Hz, 1H), 6.64 (d, J=5.2 Hz, 1H), 6.37 (s, 2H), 5.56 (s, 1H), 4.04 (q, J=7.1 Hz, 1H), 2.00 (s, 1H), 1.77 (d, J=12.4 Hz, 2H), 1.61-1.51 (m, 2H), 1.44 (td, J=11.9, 11.4, 3.6 Hz, 3H), 1.26-1.07 (m, 3H).

The obtained fluorobenzonitrile was then dissolved in ethanol (8 ml) and hydrazine, (2 mL, 1 Mol solution in Ethanol) added. The resulting mixture was stirred at reflux for 2 hours, cooled concentrated and purified by HPLC affording the target compound as a yellow powder (0.076 g, 59%). ¹H NMR (500 MHz, DMSO-d₆) δ 8.18 (d, J=1.6 Hz, 1H), 8.03 (d, J=6.4 Hz, 1H), 7.80 (s, 2H), 7.68 (dd, J=8.7, 1.7 Hz, 1H), 7.36 (d, J=8.7 Hz, 1H), 6.99 (d, J=6.5 Hz, 1H), 1.81 (dt, J=9.2, 5.0 Hz, 2H), 1.53-1.41 (m, 4H), 1.36-1.28 (m, 1H), 1.26-1.05 (m, 3H). LRMS (ESI) m/z [M]⁺. C₂₀H₂₁N₅O Molecular Weight: 347.4220 found 348.1

### Example 91

### 1-((2-Amino-4-chloropyridin-3-yl)ethynyl)cycloheptan-1-ol

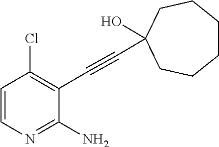 [SMILES: Nc1nccc(Cl)c1C#CC1(O)CCCCCC1]

A solution of 4-chloro-3-iodo-pyridine-2-amine (0.383 g, 1.50 mmol), copper (1) iodide (0.029 g, 0.150 mmol) and bis(triphenylphosphine) palladium(II) chloride (0.0103 g, 0.150 mmol) in 5 ml of N,N-dimethyformamide-triethylamine (1:4) was degassed in sealed tube followed by addition of 1-ethynylcycloheptan-1-ol (0.250 g, 1.81 mmol). The reaction mixture was stirred at 80° C. for 4 h. The reaction mixture was then diluted with EtOAc and extracted with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a light brown solid (0.251 g, 63%). ¹H NMR (500 MHz, DMSO-d₆) δ 7.96 (s, 1H), 7.87 (d, J=5.4 Hz, 2H), 6.70 (d, J=5.4 Hz, 2H), 6.46 (s, 3H), 5.49 (s, 2H), 2.00 (dd, J=13.8, 7.3 Hz, 2H), 1.85-1.72 (m, 2H), 1.68-1.46 (m, 8H). LRMS (ESI) m/z [M]⁺. C₁₄H₁₇ClN₂O Molecular Weight: 264.7530 found 265.1

### 1-((2-Amino-4-(3-amino-1H-indazol-5-yl)pyridine-3-yl)ethynyl)cycloheptan-1-ol

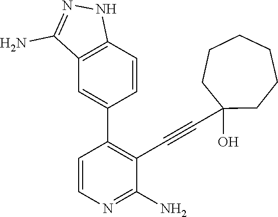 [SMILES: Nc1nccc(-c2ccc3[nH]nc(N)c3c2)c1C#CC1(O)CCCCCC1]

1-((2-Amino-4-chloropyridin-3-yl)ethynyl)cycloheptan-1-ol (0.251 g, 0.946 mmol), 2-fluoro-5-(4,4,5,5-tetramethyl-1,3,2-dioxaborolan-2-yl)benzonitrile (0.3508 g, 1.42 mmol), [1,1′-bis(di-tert-butylphosphino)ferrocene]dichloropalladium(II) catalyst (0.0308 g, 0.0473 mmol, 5 mol %), cesium carbonate (1.079 g, 3.313 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-((1-hydroxycyclohexyl)ethynyl)pyridin-4-yl)-2-fluorobenzonitrile (0.332 g, 88%). ¹H NMR (500 MHz, DMSO-d₆) δ 8.16 (dd, J=6.2, 2.3 Hz, 1H), 8.00 (d, J=5.2 Hz, 1H), 7.96 (ddd, J=8.7, 5.3, 2.4 Hz, 1H), 7.64 (t, J=9.1 Hz, 1H), 6.65 (d, J=5.2 Hz, 1H), 6.36 (s, 2H), 5.42 (s, 1H), 1.87 (dd, J=13.7, 8.2 Hz, 2H), 1.74-1.66 (m, 2H), 1.57-1.43 (m, 6H), 1.26 (td, J=9.3, 8.8, 4.6 Hz, 2H).

The obtained fluorobenzonitrile was then dissolved in ethanol (8 ml) and hydrazine, (2 mL, 1 Mol solution in ethanol) added. The resulting mixture was stirred at reflux for 2 hours, cooled, concentrated and purified by HPLC affording the target compound as a yellow powder (0.049 g, 14%). ¹H NMR (500 MHz, DMSO-d₆) δ 8.18 (d, J=1.6 Hz, 1H), 8.02 (d, J=6.4 Hz, 1H), 7.67 (dd, J=8.7, 1.7 Hz, 1H), 7.35 (d, J=8.7 Hz, 1H), 7.15 (d, J=51.1 Hz, 2H), 7.02-6.96 (m, 2H), 1.96-1.89 (m, 2H), 1.77-1.68 (m, 3H), 1.50-1.37 (m, 6H), 1.27 (d, J=15.3 Hz, 3H). LRMS (ESI) m/z [M]⁺. C₂₁H₂₃N₅O Molecular Weight: 361.4490 found 362.3

### Example 92

### 3-(3-Amino-3-methylbut-1-yn-1-yl)-4-chloropyridin-2-amine

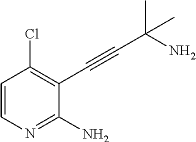

A solution of 4-chloro-3-iodo-pyridine-2-amine (0.3 g, 1.181 mmol), copper (I) iodide (0.023 g, 0.1181 mmol) and bis(triphenylphosphine) palladium(II) chloride (0.083 g, 0.1181 mmol) in 5 ml of N,N-dimethyformamide-triethylamine (1:4) was degassed in sealed tube followed by addition of 2 y 3--2mine (0.116 g, 0.147 mL, 1.42 mmol). The reaction mixture was stirred at 80° C. for 4 h. The reaction mixture was then diluted with EtOAc and extract with 1M sodium carbonate. The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as (0.1941 g, 78%). ¹H NMR (500 MHz, DMSO-d₆) δ 7.84 (d, J=5.5 Hz, 1H), 6.67 (d, J=5.5 Hz, 1H), 6.48 (s, 2H), 2.90 (s, 2H), 1.40 (s, 6H). LRMS (ESI) m/z [M]⁺. C₁₀H₁₂ClN₃ Molecular Weight: 209.6770 found 210.1

### 5-(2-Amino-3-(3-amino-3-methylbut-1-yn-1-yl)pyridin-4-yl)-1H-indazol-3-amine

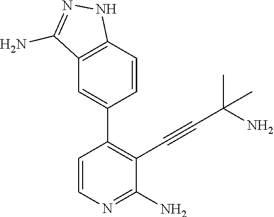 [SMILES: CC(C)(N)C#Cc1c(-c2ccc3[nH]nc(N)c3c2)ccnc1N]

3-(3-Amino-3-methylbut-1-yn-1-yl)-4-chloropyridin-2-amine (0.194 g, 0.93 mmol), 2-fluoro-5-(4,4,5,5-tetramethyl-1,3,2-dioxaborolan-2-yl)benzonitrile (0.275 g, 1.11 mmol), [1,1′-bis(di-tert-butylphosphino)ferrocene]dichloropalladium(II) catalyst (0.0325 g, 0.0464 mmol, 5 mol %), cesium carbonate (0.907 g, 2.784 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 5-(2-amino-3-((1-hydroxycyclohexyl)ethynyl)pyridin-4-yl)-2-fluorobenzonitrile (0.199 g, 88%). ¹H NMR (500 MHz, DMSO-d₆) δ 8.21 (dd, J=6.2, 2.4 Hz, 1H), 8.03 (ddd, J=8.8, 5.2, 2.4 Hz, 1H), 7.98 (d, J=5.2 Hz, 1H), 7.64 (t, J=9.1 Hz, 1H), 6.67 (d, J=5.2 Hz, 1H), 6.35 (s, 2H), 1.29 (s, 6H).

The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.149 g, 54%). ¹H NMR (400 MHz, DMSO-d₆) δ 8.51 (s, 4H), 8.22 (d, J=1.7 Hz, 1H), 8.07 (d, J=6.1 Hz, 1H), 7.65 (dd, J=8.7, 1.8 Hz, 1H), 7.36 (d, J=8.8 Hz, 1H), 6.93 (d, J=6.2 Hz, 1H), 2.54 (t, J=5.5 Hz, 2H), 1.55 (s, 6H). LRMS (ESI) m/z [M]⁺. C₁₇H₁₈N₆ Molecular Weight: 306.3730 found 307.1

### Example 93

### 4-Chloro-3-(4-(piperidin-1-yl)but-1-yn-1yl)pyridin-2-amine

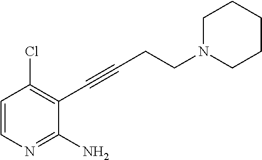 [SMILES: Nc1nccc(Cl)c1C#CCCN1CCCCC1]

A solution of 4-chloro-3-iodo-pyridine-2-amine (0.15 g, 0.6 mmol), copper (I) iodide (0.005 g, 0.03 mmol) and bis(triphenylphosphine) palladium(II) chloride (0.02 g, 0.03 mmol) in 4 ml of N,N-dimethyformamide-triethylamine (1:4) was degassed in sealed tube followed by addition of 4-(but-3-yn-1-yl)piperidine (0.12 ml, 0.88 mmol). The reaction mixture was stirred at 80° C. for 4 h. The reaction mixture was then diluted with EtOAc and extracted with 1M sodium carbonate.

The extracted organic layer was washed with brine and dried over magnesium sulfate and concentrated under reduced pressure and the residue purified by column chromatography (80% EtOAc and 1% triethylamine in petroleum ether 60-80%) to give the product as yellow oil (0.063 g, 40%), 1H NMR (400 MHz, DMSO-d₆) δ ppm 1.39 (d, J=5.19 Hz, 2H) 1.52 (quin, J=5.57 Hz, 4H) 2.39 (br. s., 4H) 2.52-2.55 (m, 2H) 2.64-2.72 (m, 2H) 6.65 (d, J=5.34 Hz, 3H) 6.83 (d, J=5.49 Hz, 1H). ¹³C NMR (100 MHz, DMSO-d₆) δ ppm 18.07, 24.52, 25.83, 54.08, 57.34, 74.94, 101.20, 102.19, 112.47, 143.22, 148.05, 161.96. m/z (ESI-MS) [M]⁺ 264.0.

### 5-(2-Amino-3-(4-(piperidin-1-yl)but-1-yn-1-yl)pyridin-4-yl)-1H-indazol-3-amine

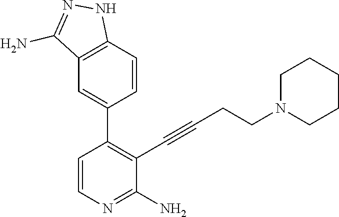 [SMILES: Nc1nccc(-c2ccc3[nH]nc(N)c3c2)c1C#CCCN1CCCCC1]

4-Chloro-3-(4-(piperidin-1-yl)but-1-yn-1-yl)pyridin-2-amine (0.075 g, 0.28 mmol), 5-(4,4,5,5-tetramethyl-1,3,2-dioxaborolan-2-yl)-1H-indazol-3-amine (97) (0.115 g, 0.42 mmol), 1M potassium phosphate solution (0.6 ml, 0.6 mmol), [1,1′-bis(di-tert-butylphosphino)ferrocene]dichloropalladium(II) catalyst (0.003 g, 0.014 mmol) in 1.7 ml ethanol were reacted as described in General procedure A and chromatographic purification (90% EtOAc and 1% triethylamine in petroleum ether 60-80%), gave the titled compound as yellow solid (63.3 mg, 35%). ¹H NMR (400 MHz, DMSO-d₆) δ ppm 1.31-1.42 (m, 2H) 1.42-1.55 (m, 4H) 2.33 (br. s., 4H) 2.45 (d, J=5.71 Hz, 2H) 2.52-2.59 (m, 2H) 5.44 (s, 2H) 6.42 (s, 2H) 6.58 (d, J=5.27 Hz, 1H) 7.26 (d, J=8.79 Hz, 1H) 7.53 (dd, J=8.79, 1.32 Hz, 1H) 7.90 (d, J=5.27 Hz, 1H) 7.96 (s, 1H) 11.49 (s, 1H). ¹³C NMR (100 MHz, DMSO-d₆) δ ppm 18.09, 25.85, 54.09, 57.40, 60.32, 77.85, 98.27, 100.45, 109.35, 112.97, 114.58, 121.15, 127.48, 128.63, 141.61, 147.12, 150.32, 151.02, 161.73. m/z (ESI-HRMS) calculated for C₂₁H₂₅N₆=361.2135 found=361.2132.

### Example 94

### 4-Chloro-3-(5-morpholinopent-1-yn-1yl)pyridin-2-amine

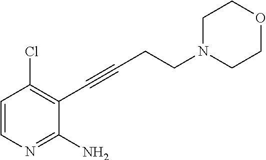 [SMILES: Nc1nccc(Cl)c1C#CCCN1CCOCC1]

A solution of 4-chloro-3-iodo-pyridine-2-amine (0.15 g, 0.6 mmol), copper (I) iodide (0.005 g, 0.03 mmol) and bis(triphenylphosphine) palladium(II) chloride (0.02 g, 0.03 mmol) in 4 ml of N,N-dimethyformamide-triethylamine (1:4) was degassed in sealed tube followed by addition of 4-(but-3-yn-1-yl)morpholine (122) (0.12 ml, 0.88 mmol). The reaction mixture was stirred at 80° C. for 3 h. The reaction mixture was diluted with EtOAc and extracted with 1 M sodium carbonate. The extracted organic layer was washed with brine and dried over magnesium sulfate and concentrated under reduced pressure and the residue purified by column chromatography (80% EtOAc and 1% triethylamine in petroleum ether 60-80%) to give the product as yellow oil (0.139 g, 89%). 1H NMR (400 MHz, DMSO-d₆) δ ppm 2.43 (br. s., 4H) 2.56 (t, J=6.81 Hz, 2H) 3.70 (t, J=6.81 Hz, 2H) 3.59 (t, J=4.61 Hz, 4H) 6.51-6.78 (m, 3H) 6.84 (d, J=5.27 Hz, 1H). ¹³C NMR (100 MHz, DMSO-d₆) δ ppm 17.85, 53.45, 57.05, 66.58, 75.05, 101.03, 102.25, 112.61, 143.38, 148.20, 162.01. m/z (ESI-MS) [M]⁺ 266.0.

### 5-(2-Amino-3-(4-morpholinobut-1-yn-1-yl)pyridin-4-yl)-1H-indazol-3-amine

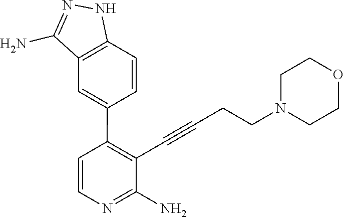 [SMILES: Nc1nccc(-c2ccc3[nH]nc(N)c3c2)c1C#CCCN1CCOCC1]

4-Chloro-3-(4-morpholinobut-1-yn-1-yl)pyridin-2-amine (0.029 g, 0.1 mmol), 5-(4,4,5,5-tetramethyl-1,3,2-dioxaborolan-2-yl)-1H-indazol-3-amine (97) (0.042 g, 0.16 mmol), 1M potassium phosphate solution (0.2 ml, 0.2 mmol), [1,1′-bis(di-tert-butylphosphino)ferrocene]dichloropalladium(II) catalyst (0.001 g, 0.005 mmol) in 0.6 ml ethanol were reacted as described in General procedure A and chromatographic purification (90% EtOAc and 1% triethylamine in petroleum ether 60-80%), gave the titled compound as yellow solid (28 mg, 78%). ¹H NMR (400 MHz, DMSO-d₆) δ ppm 2.36 (br. s., 4H) 2.42-2.48 (m, 2H) 2.53-2.60 (m, 2H) 3.55 (t, J=4.39 Hz, 4H) 5.45 (s, 2H) 6.43 (s, 2H) 6.58 (d, J=5.27 Hz, 1H) 7.26 (d, J=8.79 Hz, 1H) 7.49-7.56 (m, 1H) 7.91 (d, J=5.27 Hz, 1H) 7.96 (s, 1H) 11.49 (s, 1H). ¹³C NMR (100 MHz, DMSO-d₆) δ ppm 17.98, 53.40, 57.07, 66.53, 71.18, 98.07, 100.43, 109.36, 113.00, 121.15, 123.89, 127.47, 128.60, 141.60, 147.15, 150.33, 152.06, 161.69. m/z (ESI-HRMS) calculated for C₂₀H₂₃ON₆=363.1928 found=363.1925.

### Example 95

### 4-Chloro-3-(5-chloropent-1-yn-1yl)pyridin-2-amine

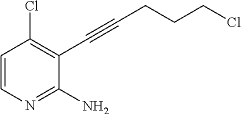 [SMILES: Nc1nccc(Cl)c1C#CCCCCl]

A solution of 4-chloro-3-iodo-pyridine-2-amine (0.15 g, 0.6 mmol), copper (I) iodide (0.005 g, 0.03 mmol) and bis(triphenylphosphine) palladium(II) chloride (0.02 g, 0.03 mmol) in 4 ml of tetrahydrofuran—triethylamine (1:4) was degassed in sealed tube followed by addition of 5-chloro-1-pentyne (0.1 ml, 0.9 mmol). The reaction mixture was stirred at room temperature for 16 h. The reaction mixture was diluted with EtOAc and extracted with water. The extracted organic layer was dried over magnesium sulfate and concentrated under reduced pressure.

The crude then purified using column chromatography (10% EtOAc in petroleum ether 60-80%) to give the product as yellow oil (0.102 g, 75%). 1H NMR (400 MHz, DMSO-d₆) δ ppm 2.03 (quin, J=6.70 Hz, 2H) 2.67 (t, J=6.81 Hz, 2H) 3.79 (t, J=6.59 Hz, 2H) 6.48 (br.s, 2H) 6.67 (d, J=5.27 Hz, 1H) 6.84 (d, J=4.83 Hz, 1H). ¹³C NMR (100 MHz, DMSO-d₆) δ ppm 17.48, 31.37, 44.58, 74.36, 100.42, 102.02, 112.80, 144.48, 148.25, 161.55. m/z (ESI-MS) [M]⁺ 229.0.

### 4-Chloro-3-(5-piperdinopent-1-yn-1yl)pyridin-2-amine

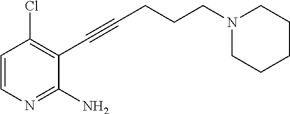 [SMILES: Nc1nccc(Cl)c1C#CCCCN1CCCCC1]

In sealed tube, a mixture of 4-chloro-3-(5-chloropent-1-yn-1yl)pyridin-2-amine (0.1 g, 0.4 mmol), piperdine (0.5 ml) and potassium iodide (0.04 g, 0.22 mmol) in N,N-dimethylamide (1.5 ml) was heated to 110° C. for 50 min. After the reaction was cooled to room temperature, EtOAc and 1 M sodium carbonate solution were added. Extracted organic layer was dried over magnesium sulfate and concentrated under reduced pressure to give the product as yellow oil (0.1 g, 90%). 1H NMR (400 MHz, DMSO-d₆) δ ppm 1.37 (d, J=5.49 Hz, 2H) 1.48 (quin, J=5.49 Hz, 4H) 2.03 (quin, J=7.05 Hz, 2H) 2.31 (br. s., 4H) 2.36 (t, J=7.14 Hz, 2H) 2.67 (t, J=4.61 Hz, 2H) 6.41 (br. s., 2H) 6.66 (dd, J=5.49, 2.93 Hz, 1H) 7.83 (d, J=5.49 Hz, 1H). ¹³C NMR (100 MHz, DMSO-d₆) δ ppm 16.88, 25.58, 30.77, 44.24, 54.10, 57.43, 73.76, 101.61, 102.30, 112.19, 143.70, 147.65, 160.95. m/z (ESI-MS) [M]⁺ 292.1. 5-(2-Amino-3-(5-(piperidin-1-yl)pent-1-yn-1-yl)pyridin-4-yl)-1H-indazol-3-amine

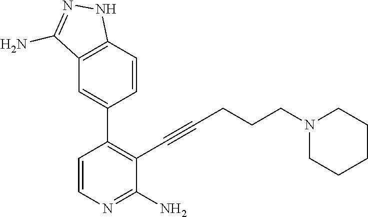 [SMILES: Nc1nccc(-c2ccc3[nH]nc(N)c3c2)c1C#CCCCN1CCCCC1]

4-Chloro-3-(4-(piperidin-1-yl)pent-1-yn-1-yl)pyridin-2-amine (0.087 g, 0.31 mmol), 5-(4,4,5,5-tetramethyl-1,3,2-dioxaborolan-2-yl)-1H-indazol-3-amine (97) (0.122 g, 0.47 mmol), 1M potassium phosphate solution (0.6 ml, 0.6 mmol), [1,1′-bis(di-tert-butylphosphino)ferrocene]dichloropalladium(II) catalyst (0.003 g, 0.015 mmol) in 1.8 ml ethanol were reacted as described in General procedure A and chromatographic purification (90% EtOAc and 1% triethylamine in petroleum ether 60-80%), gave the titled compound as yellow solid (48 mg, 42%). ¹H NMR (400 MHz, DMSO-d₆) δ ppm 1.34 (br. s., 2H) 1.39-1.49 (m, 4H) 1.54-1.67 (m, 2H) 2.18 (br. s., 6H) 2.39 (t, J=6.81 Hz, 2H) 5.43 (br. s., 2H) 6.10 (br. s., 2H) 6.59 (d, J=5.27 Hz, 1H) 7.25 (d, J=8.79 Hz, 1H) 7.51 (dd, J=8.57, 1.10 Hz, 1H) 7.91 (d, J=5.27 Hz, 1H) 7.94 (s, 1H) 11.49 (s, 1H). ¹³C NMR (100 MHz, DMSO-d₆) δ ppm 24.43, 25.65, 45.57, 51.56, 52.49, 54.38, 59.87, 91.29, 100.62, 109.26, 113.36, 114.55, 121.19, 127.55, 128.62, 141.57, 147.05, 150.30, 151.91, 161.11. m/z (ESI-HRMS) calculated for C₂₂H₂₇N_{g}=375.2292 found=375.2292.

### Example 96

### 4-Chloro-3-(5-morpholinopent-1-yn-1yl)pyridin-2-amine

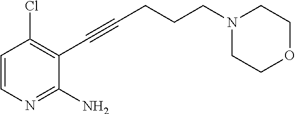 [SMILES: Nc1nccc(Cl)c1C#CCCCN1CCOCC1]

In sealed tube, a mixture of 4-chloro-3-(5-chloropent-1-yn-1yl)pyridin-2-amine (0.1 g, 0.4 mmol), morpholine (0.5 ml) and potassium iodide (0.04 g, 0.22 mmol) in N,N-dimethylamide (1.5 ml) was heated to 110° C. for 50 min. After the reaction was cooled to room temperature, EtOAc and 1 M sodium carbonate solution were added. Extracted organic layer was dried over magnesium sulfate and concentrated under reduced pressure to give the product as yellow oil (115 mg, 90%). 1H NMR (400 MHz, DMSO-d₆) δ ppm 1.73 (quin, J=7.14 Hz, 2H) 2.35 (br. s., 4H) 3.41 (t, J=7.03 Hz, 2H) 2.52-2.56 (m, 2H) 3.57 (t, J=4.61 Hz, 4H) 6.42 (br. s., 2H) 6.66 (d, J=5.71 Hz, 1H) 7.82 (d, J=5.27 Hz, 1H). ¹³C NMR (100 MHz, DMSO-d₆) δ ppm 17.77, 25.57, 53.95, 57.69, 66.77, 79.36, 100.30, 102.30, 112.82, 144.29, 148.05, 161.50. m/z (ESI-MS) [M]⁺ 280.1.

### 5-(2-Amino-3-(5-morpholinopent-1-yn-1-yl)pyridin-4-yl)-1H-indazol-3-amine

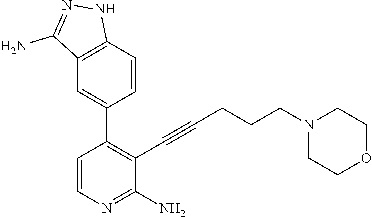 [SMILES: Nc1nccc(-c2ccc3[nH]nc(N)c3c2)c1C#CCCCN1CCOCC1]

4-Chloro-3-(5-morpholinopent-1-yn-1yl)pyridin-2-amine (0.11 g, 0.4 mmol), 5-(4,4,5,5-tetramethyl-1,3,2-dioxaborolan-2-yl)-1H-indazol-3-amine (97) (0.15 g, 0.6 mmol), 1M potassium phosphate solution (0.8 ml, 0.8 mmol), [1,1′-bis(di-tert-butylphosphino)ferrocene]dichloropalladium(II) catalyst (0.005 g, 0.02 mmol) in 2.4 ml ethanol were reacted as described in General procedure A and chromatographic purification (90% EtOAc in petroleum ether 60-80%), gave the titled compound as green crystals (102 mg, 68%). ¹H NMR (400 MHz, DMSO-d₆) δ ppm 1.58 (quin, J=7.03 Hz, 2H) 2.11-2.20 (m, 6H) 2.40 (t, J=6.81 Hz, 2H) 3.49 (t, J=4.61 Hz, 4H) 5.43 (s, 2H) 6.10 (s, 2H) 6.59 (d, J=5.27 Hz, 1H) 7.20-7.29 (m, 1H) 7.50 (dd, J=8.79, 1.76 Hz, 1H) 7.90 (d, J=4.83 Hz, 1H) 7.94 (s, 1H) 11.48 (s, 1H). ¹³C NMR (100 MHz, DMSO-d₆) δ ppm 17.71, 25.43, 53.79, 57.61, 66.72, 76.73, 99.22, 100.67, 109.27, 113.36, 114.54, 121.19, 127.54, 128.62, 141.57, 147.03, 150.31, 151.96, 161.09. m/z (ESI-HRMS) calculated for C₂₀H₂₃ON₇=377.1959 found=377.1959.

### Example 97

### tert-Butyl 4-((2-amino-4-chloropyridin-3-yl)ethynyl)methyl)piperidine-1-carboxylate

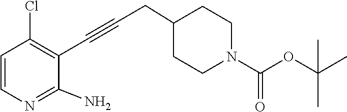 [SMILES: CC(C)(C)OC(=O)N1CCC(CC#Cc2c(Cl)ccnc2N)CC1]

A solution of 4-chloro-3-iodo-pyridine-2-amine (0.12 g, 0.48 mmol), copper (I) iodide (0.004 g, 0.024 mmol) and bis(triphenylphosphine) palladium(II) chloride (0.016 g, 0.024 mmol) in 3 ml of N,N-dimethyformamide-triethylamine (1:4) was degassed in sealed tube followed by addition of tert-butyl 4-(prop-2-yn-1-yl)piperidine-1-carboxylate (0.13 g, 0.58 mmol). The reaction mixture was stirred at 80° C. for 4 h. The reaction mixture was then diluted with EtOAc and extracted with 1 M sodium carbonate. The extracted organic layer was washed with brine and dried over magnesium sulfate and concentrated under reduced pressure and the residue purified by column chromatography (20% EtOAc in petroleum ether 60-80%) to give the product as yellow oil (0.13 g, 77%), ¹H NMR (400 MHz, DMSO-d₆) δ ppm 1.11-1.28 (m, 4H) 1.39 (s, 9H) 1.43-1.45 (m, 1H) 1.74 (d, J=9.67 Hz, 4H) 3.96 (d, J=12.30 Hz, 2H) 6.41 (br. s., 2H) 6.67 (d, J=5.71 Hz, 1H) 7.82 (d, J=5.27 Hz, 1H). ¹³C NMR (100 MHz, DMSO-d₆) b ppm 26.62, 28.66, 31.33, 35.35, 36.34, 74.92, 79.02, 100.39, 102.25, 112.84, 144.39, 148.11, 154.42, 161.47. m/z (ESI-MS) [M-tert-butyl]⁺ 294.0.

### 5-(2-Amino-3-(3-(piperidin-4-yl)prop-1-yn-1-yl)pyridin-4-yl)-1H-indazol-3-amine

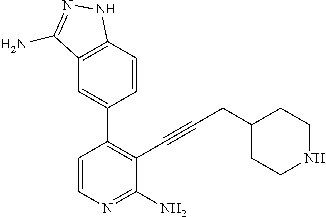 [SMILES: Nc1nccc(-c2ccc3[nH]nc(N)c3c2)c1C#CCC1CCNCC1]

tert-Butyl 4-((2-amino-4-chloropyridin-3-yl)ethynyl)methyl)piperidine-1-carboxylate (0.012 g, 0.048 mmol), 5-(4,4,5,5-tetramethyl-1,3,2-dioxaborolan-2-yl)-1H-indazol-3-amine (0.018 g, 0.072 mmol), 1M potassium phosphate solution (0.1 ml, 0.1 mmol), [1,1′-bis(di-tert-butylphosphino)ferrocene]dichloropalladium(II) catalyst (0.002 g, 0.002 mmol) in 0.3 ml ethanol were reacted as described in general procedure A. The crude product was dissolved in THF (20 mL) and TBAF (5 mL, 1M solution in THF) added and refluxed for 8 hours, cooled, concentrated and purified by HPLC to give the titled compound as yellow solid (3.75 mg, 22%). ¹H NMR (400 MHz, DMSO-d₆) δ ppm 1.29-1.41 (m, 2H) 1.75-1.87 (m, 3H) 2.51 (d, J=6.71 Hz, 2H) 2.74-2.83 (m, 2H) 3.23 (d, J=12.36 Hz, 2H) 7.00 (d, J=6.41 Hz, 1H) 7.42 (d, J=8.70 Hz, 1H) 7.63 (d, J=1.53 Hz, 1H) 2.92 (br.s, 1H) 8.06 (d, J=6.56 Hz, 1H) 8.26 (s, 1H) 8.28-8.38 (m, 1H) 8.57-8.72 (m, 1H) 11.88 (br. s., 1H). ¹³C NMR (100 MHz, DMSO-d₆) b ppm 26.29, 28.33, 23.80, 43.40, 77.64, 97.87, 107.50, 109.98, 113.70, 114.24, 122.56, 127.48, 127.53, 141.94, 147.97, 150.26, 151.36, 163.86. m/z (ESI-HRMS) calculated for C₂₀H₂₃N₆=347.1979 found=347.1977.

### Example 98

### 3-(2-Amino-4-chloropyridin-3-yl)-N-methylpropiolamide

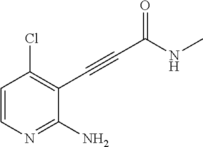

A solution of 4-chloro-3-iodo-pyridine-2-amine (0.15 g, 0.59 mmol), copper (I) iodide (0.006 g, 0.03 mmol) and bis(triphenylphosphine) palladium(II) chloride (0.004 g, 0.03 mmol) in 3 ml of N,N-dimethyformamide-triethylamine (1:4) was degassed in sealed tube followed by addition of N-methylpropiolamide (0.059 g, 0.7089 mmol). The reaction mixture was stirred at 80° C. for 4 h. The reaction mixture was then diluted with EtOAc and extract with 1M sodium carbonate.

The extracted organic layer was washed with brine and dried over anhydrous magnesium sulfate and concentrated under reduced pressure and the residue purified by column chromatography (50% EtOAc and 1% triethylamine in petroleum ether 60-80%) to give the product as a dark brown oil (0.073 g, 59%). ¹H NMR (500 MHz, DMSO-d₆) δ 8.77 (d, J=5.1 Hz, 1H), 7.98 (d, J=5.4 Hz, 1H), 6.88 (s, 2H), 6.74 (d, J=5.4 Hz, 1H), 2.71 (d, J=4.8 Hz, 3H). LRMS (ESI) m/z [M]⁺. For C₉H₃ClN₃O Molecular Weight: 209.6330 found 210.5

### 3-(2-Amino-4-(3-amino-1H-indazol-5-yl)pyridin-3-yl)-N-methylpropiolamide

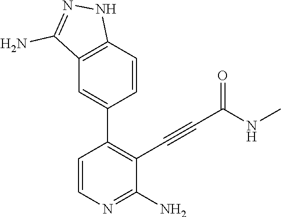 [SMILES: CNC(=O)C#Cc1c(-c2ccc3[nH]nc(N)c3c2)ccnc1N]

3-(2-Amino-4-chloropyridin-3-yl)-N-methylpropiolamide (0.073 g, 0.349 mmol), 2-fluoro-5-(4,4,5,5-tetramethyl-1,3,2-dioxaborolan-2-yl)benzonitrile (0.1035 g, 0.419 mmol), [1,1′-bis(di-tert-butylphosphino)ferrocene]dichloropalladium(II) catalyst (0.0114 g, 0.0174 mmol, 5 mol %), Cesium carbonate (0.341 g, 1.047 mmol) in a sealed tube flushed with argon was added a degassed mixture of dioxane and water (18:2 7 mL) and stirred at 110° C. for 18 hours, concentrated and purified by flash chromatography (50% EtOAc and 1% triethylamine in petroleum ether 60-80%) to afford 3-(2-amino-4-(3-cyano-4-fluorophenyl)pyridin-3-yl)-N-methylpropiolamide The obtained fluorobenzonitrile was then dissolved in ethanol (8 ml) and hydrazine, (2 mL, 1 Mol solution in ethanol) added. The resulting mixture was stirred at reflux for 2 hours, cooled concentrated and purified by HPLC affording the target compound as a yellow powder (0.015 g, 14%). ¹H NMR (500 MHz, DMSO-d₆) δ 12.27 (s, 1H), 8.54 (d, J=4.8 Hz, 2H), 8.39 (d, J=4.9 Hz, 1H), 8.13 (d, J=2.4 Hz, 1H), 8.08 (dd, J=8.8, 2.3 Hz, 2H), 7.45 (d, J=8.8 Hz, 1H), 7.32 (d, J=2.1 Hz, 1H), 7.28 (d, J=4.9 Hz, 1H), 2.83 (d, J=4.6 Hz, 3H). LRMS (ESI) m/z [M]⁺. C₁₅H₁₄N₆O Molecular Weight: 306.3290 found 307.1

### Example 99

### 5-(2-Amino-4-(3-amino-1H-indazol-5-yl)pyridine-3-yl)-1-morpholinopent-4-yn-1-one

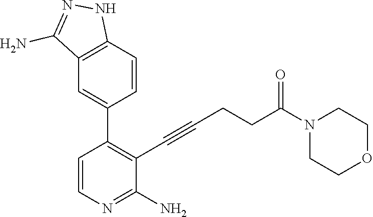 [SMILES: Nc1nccc(-c2ccc3[nH]nc(N)c3c2)c1C#CCCC(=O)N1CCOCC1]

A 2-5 mL Biotage MW tube was charged with 5-(2-amino-4-chloropyridin-3-yl)-1-morpholinopent-4-yn-1-one (100 mg, 0.3 mmol), 5-(4,4,5,5-tetramethyl-1,3,2-dioxaborolan-2-yl)-1H-indazol-3-amine (103 mg, 0.4 mmol, 1.3 eq.), [1,1′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃PO₄ (1.8 mL). The reaction mixture was heated to 110° C. for 16 hrs. The reaction mixture was then allowed to cool to room temperature, before being diluted with water (10 mL) and extracted with ethyl acetate (3×20 mL). The combined organic fractions were then dried over anhydrous magnesium sulfate, filtered and the solvent removed under reduced pressure. The crude product was then purified by HPLC to give the desired product as a pale yellow solid (14 mg). ¹H NMR (500 MHz, DMSO-D₆) δ 1.12 (m, 4H), 1.64 (m, 4H), 2.38 (t, J=6.9 Hz, 1H), 2.66 (t, J=7.0 Hz, 1H), 6.96 (d, J=6.6 Hz, 1H), 7.31 (d, J=8.7 Hz, 1H), 7.68 (dd, J=8.4 and 1.6 Hz, 1H), 7.84 (d, J=7.8 Hz, 1H), 8.24 (s, 1H). LRMS: Calculated for C₂₁H₂₂NO₂ 390.18; Found: 391.20.

### Example 100

### 5-(2-Amino-3-cyclopropylpyridin-4-yl)-1H-indazol-3-amine

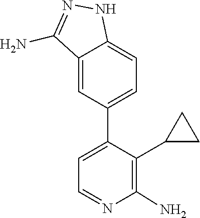 [SMILES: Nc1nccc(-c2ccc3[nH]nc(N)c3c2)c1C1CC1]

A 0.5-2 mL MW tube was charged with a mixture of 4-chloro-3-cyclopropylpyridin-2-amine (29.5 mg, 0.175 mmol, 1 eq.) and 5-(4,4,5,5-tetramethyl-1,3,2-dioxaborolan-2-yl)-1H-indazol-3-amine (59 mg, 0.228 mmol, 1.3 eq.), [1,1′-bis(di-tert-butylphosphino)ferrocene]dichloropalladium(II) catalyst 5.7 mg, 0.00875 mmol, in EtOH (0.7 mL) was stirred under argon before adding 1 M aq. K₃PO₄ (0.35 mL). The reaction was heated to 50° C. under a gentle flow of argon for 10 minutes, then at 100° C. for 24 hours. The stirred reaction mixture was then cooled to room temperature and slowly diluted with water (7 mL) to precipitate a solid, which was filtered and washed with 1 M aq. Na₂CO₃ (10 mL×3), water (10 mL) and hexane (2 mL) to give the title compound as a beige solid (26 mg).

¹H NMR (400 MHz, DMSO-D₆) δ −0.18-−0.10 (m, 2H), 0.66-0.75 (m, 2H), 1.63-1.75 (m, 1H), 5.39 (s, 2H), 5.79 (s, 2H), 6.47 (d, J=5.2 Hz, 1H), 7.23 (d, J=8.5 Hz, 1H), 7.33 (dd, J=8.6, 1.6 Hz, 1H), 7.76 (s, 1H), 7.82 (d, J=5.2 Hz, 1H), 11.42 (s, 1H).

### Example 101

### 4-(2-Amino-4-(3-amino-1H-indazol-5-yl)pyridin-3-yl)butan-1-ol

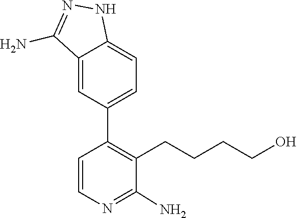 [SMILES: Nc1nccc(-c2ccc3[nH]nc(N)c3c2)c1CCCCO]

To a solution of 4-(2-amino-4-(3-amino-1H-indazol-5-yl)pyridin-3-yl)but-3-yn-1-ol (Example 84 SU1503) (42 mg, 0.14 mmol) in 1.2 ml of dry MeOH was added 10% Pd/C (20 mg, 0.19 mmol). The reaction was stirred at RT, o/n under a hydrogen atmosphere. The resulting mixture was diluted with EtOAc, filtered through silica, concentrated under reduced pressure and the residue purified by HPLC to afford the title compound as a white solid (21 mg, 0.07 mmol, 52%). ¹H NMR (400 MHz, MeOD): δ 1.36-1.50 (m, 2H), 1.54-1.66 (m, 2H), 2.60-2.71 (m, 2H), 3.44 (t, J=6.3 Hz, 2H), 6.81 (d, J=6.3 Hz, 1H), 7.29 (dd, J=8.6, 1.7 Hz, 1H), 7.42 (dd, J=8.6, 0.9 Hz, 1H), 7.71 (dd, J=1.7, 0.9 Hz, 1H), 7.77 (d, J=6.3 Hz, 1H), 8.30 (br s, 1H), 5 protons missing. LRMS: Calculated for C₁₉H₁₉N₅O 297.2 found 298.3 (M+1).

### Example 102

### 1-(2-(2-Amino-4-(3-amino-1H-indazol-5-yl)pyridin-3-yl)ethyl)cyclohexan-1-ol

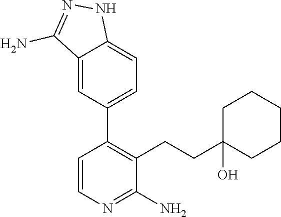 [SMILES: Nc1nccc(-c2ccc3[nH]nc(N)c3c2)c1CCC1(O)CCCCC1]

To a solution of 1-((2-amino-4-(3-amino-1H-indazol-5-yl)pyridin-3-yl)ethynyl)cyclohexan-1-ol (Example 90) (90 mg, 0.26 mmol) in 2.7 ml of dry MeOH was added 10% Pd/C (40 mg, 0.38 mmol). The reaction was stirred at RT, o/n under a hydrogen atmosphere. The resulting mixture was diluted with EtOAc, filtered through silica, concentrated under reduced pressure and the residue purified by HPLC to afford the title compound as a white solid (67 mg, 0.19 mmol, 71%). 1H NMR (400 MHz, MeOD): δ 1.20-1.51 (m, 10H), 1.58-1.67 (m, 2H), 2.55-2.64 (m, 2H), 6.77 (d, J=5.2 Hz, 1H), 7.28 (d, J=8.5 Hz, 1H), 7.42 (d, J=8.5 Hz, 1H), 7.70 (s, 1H), 7.76 (d, J=5.2 Hz, 1H), 8.56 (br s, 1H), 4 protons missing. LRMS: Calculated for C₂₀H₂₅N₅O 351.2 found 352.2 (M+1).

### Example 103

### 5-(2-Amino-4-(3-amino-1H-indazol-5-yl)pyridin-3-yl)pentan-1-ol

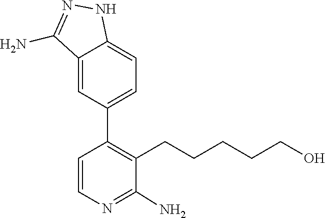 [SMILES: Nc1nccc(-c2ccc3[nH]nc(N)c3c2)c1CCCCCO]

To a solution of 5-(2-amino-4-(3-amino-1H-indazol-5-yl)pyridin-3-yl)pent-4-yn-1-ol (Example 85) (68 mg, 0.22 mmol) in 1.7 ml of dry MeOH was added 10% Pd/C (35 mg, 0.33 mmol). The reaction was stirred at RT, o/n under a hydrogen atmosphere. The resulting mixture was diluted with EtOAc, filtered through silica, concentrated under reduced pressure and the residue purified by HPLC to afford the title compound as a white solid (45 mg, 0.15 mmol, 65%). ¹H NMR (400 MHz, MeOD): δ 1.19-1.29 (m, 2H), 1.31-1.41 (m, 2H), 1.45-1.56 (m, 2H), 2.54-2.61 (m, 2H), 3.41 (t, J=6.4 Hz, 2H), 6.68 (d, J=5.8 Hz, 1H), 7.25 (dd, J=8.6, 1.6 Hz, 1H), 7.39 (dd, J=8.6, 0.9 Hz, 1H), 7.66 (dd, J=1.6, 0.9 Hz, 1H), 7.77 (d, J=5.8 Hz, 1H), 6 protons missing. LRMS: Calculated for C₁₇H₂₁N₅O 311.2 found 312.2 (M+1).

### Section 7—Compounds of the Formula

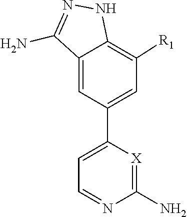

### General Method for Preparation of 7-chloro-5-(4,4,5,5-tetramethyl-1,3,2-dioxaborolan-2-yl)-1H-indazol-3-amine

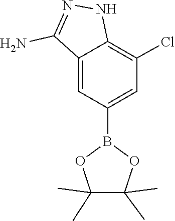 [SMILES: CC1(C)OB(c2cc(Cl)c3[nH]nc(N)c3c2)OC1(C)C]

To a solution of 3-chloro-2-fluoro-5-(4,4,5,5-tetramethyl-1,3,2-dioxaborolan-2-yl)benzonitrile (0.4 g, 1.4 mmol) in ethanol (20 ml), hydrazine hydrate (0.22 ml, 2.47 g, 6.8 mmol, 50-60%) was added and the reaction stirred at reflux for 18 h. The solvent was evaporated and the residue was triturated with 12 ml EtOAc and petroleum ether (1:1) and then filtered under vacuum and washed with water then petroleum ether 60-80% to give the desired compound as a yellow solid (320 mg, 75%). ¹H NMR (500 MHz, DMSO-d₆) δ 12.01 (s, 1H), 8.18-8.12 (m, 1H), 7.45 (s, 1H), 5.68 (s, 2H), 1.31 (s, 12H).

### General Method for Preparation of 3-bromo-2-fluoro-5-(4,4,5,5-tetramethyl-1,3,2-dioxaborolan-2-yl)benzonitrile

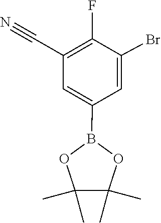 [SMILES: CC1(C)OB(c2cc(Br)c(F)c(C#N)c2)OC1(C)C]

A solution of bis(pinacolato)diboron (1.5 g, 5.99 mmol, 1.2 Eq), 4,4′-di-tert-butyl-2,2′-dipyridyl (67 mg, 0.25 mmol, 0.05 Eq) and (1,5-cyclooctadiene)(methoxy)iridium(1) dimer (132 mg, 0.2 mmol, 0.04 Eq) in anhydrous MTBE (10 mL) in a sealed vial (20 mL) was stirred at rt for 1 h. Then a solution of 3-bromo-2-fluorobenzonitrile (1 g, 5 mmol, 1 Eq) in anhydrous MTBE (2 mL) was added and the resulting reaction mixture was allowed to stir at 80° C. for 18 h. The reaction was evaporated under reduced pressure. The crude residue was purified by flash column chromatography (eluting with a gradient of diethyl ether:petroleum ether 0% to 10%) to give the titled product as a white solid (1.3 g, 4.0 mmol, 80%). 1H NMR (500 MHz, DMSO) δ 1.32 (s, 12H), 8.05 (dd, J=6.2, 1.5 Hz, 1H), 8.16 (dd, J=7.2, 1.5 Hz, 1H). ¹⁹F NMR (471 MHz, DMSO-d₆) δ −98.28. LRMS (ESI) m/z [M]⁺ 325.03, 327.03.

### General Method for Preparation of 7-bromo-5-(4,4,5,5-tetramethyl-1,3,2-dioxaborolan-2-yl)-1H-indazol-3-amine

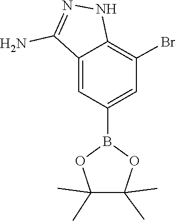 [SMILES: CC1(C)OB(c2cc(Br)c3[nH]nc(N)c3c2)OC1(C)C]

3-Bromo-2-fluoro-5-(4,4,5,5-tetramethyl-1,3,2-dioxaborolan-2-yl)benzonitrile (1 g, 3.1 mmol, 1 Eq) was stirred with hydrazine solution (1M in THF, 6.2 mL, 6.2 mmol, 2 Eq) under reflux for 18 h. The reaction mixture was concentrated under reduced pressure, cooled and extracted between EtOAc (50 mL) and water (30 mL). The organic layer was dried over anhydrous Na₂SO₄ and evaporated under reduced pressure. The crude product was washed crystalized from MeOH to give the titled product as a pale brown solid (0.8 g, 2.37 mmol, 76%). ¹H NMR (500 MHz, DMSO-d₆) δ 11.93 (s, 1H), 8.19 (s, 1H), 7.60 (s, 1H), 5.68 (s, 2H), 1.31 (s, 12H). LRMS (ESI-MS) m/z [M]⁺ 337.01 (100%), 339.01 (100%).

### General Method for the Synthesis of 5-(2-aminopyridin-4-yl)-7-bromo-3-amino-1H-indazole

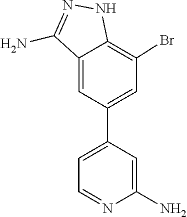 [SMILES: Nc1cc(-c2cc(Br)c3[nH]nc(N)c3c2)ccn1]

5-(4,4,5,5-Tetramethyl-1,3,2-dioxaborolan-2-yl)-7-bromo-3-amino-1H-indazole (176.1 mg, 0.6 mmol), 2-amino-4-bromopyridine (86.5 mg, 0.5 mmol) and [1,1′-bis(di-tert-butylphosphino)ferrocene]dichloropalladium(II) catalyst (16.3 mg, 0.025 mmol) were placed in a 2-5 mL microwave vial which was sealed and purged with nitrogen. Ethanol (1.5 mL) was added and the suspension brought to 50° C. with stirring before addition of K₃PO₄ (O₂-free, 1 M, 0.75 mL). Stirring continued for 24 hours. Resultant solution was diluted in EtOAc, washed with water and adsorbed onto silica under reduced pressure. Chromatographic purification (Biotage SP4, 50 g cartridge, solvent system: EtOAc/methanol, 0%, 6 CV; 0-2%, 1 CV; 2%, 3 CV; 2-10%, 1CV; 10%, 4 CV) yielded product as a yellow solid (42 mg, 32%). 1H (DMSO-d6, 400 MHz) 5 5.64 (s, 2H), 5.92 (s, 2H), 6.72 (s, 1H), 6.80 (dd, J=5.4 & 1.1 Hz, 1H), 7.71 (s, 1H), 7.95 (d, J=5.6 Hz, 1H), 8.11 (s, 1H), 11.93 (br s, 1H); LR-MS ESI 304.02 (100%), 306.02 (100%). HRMS (ESI+ve): For C₁₂H₁₁N₅Br requires 304.0198 found 304.0200.

### 5-(2-Aminopyridin-4-yl)-7-((trimethylsilyl)ethynyl)-1H-indazol3-amine (DB14/71)

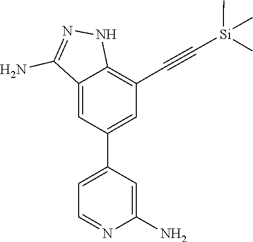 [SMILES: C[Si](C)(C)C#Cc1cc(-c2ccnc(N)c2)cc2c(N)n[nH]c12]

5-(2-Aminopyridin-4-yl)-7-bromo-3-amino-1H-indazole (0.030 g, 0.1 mmol), TMS acetylene (28 μL, 0.2 mmol) and Tetrakis(triphenylphosphine)palladium(0) catalyst (0.012 g, 0.01 mmol) and CuI (0.002 g, 0.01 mmol) were placed in a 2-5 mL microwave vial which was sealed and purged with nitrogen. DMF (1.5 mL) and Net₃ (1.5 mL) were added and the suspension brought to 60° C. with stirring and maintained for 16 hrs. Resultant solution was diluted in EtOAc, washed with water and adsorbed onto silica under reduced pressure. Chromatographic purification (Biotage SP4, 50 g cartridge, solvent system: EtOAc/methanol, 0%-10%, 1CV; 10%, 4 CV) yielded product as a yellow solid (42 mg, 32%). ¹H (DMSO-d₆, 500 MHz) δ 0.28 (s, 9H) 5.61 (s, 2H), 5.90 (s, 2H), 6.73 (s, 1H), 6.80 (s, 1H), 7.57 (s, 1H), 7.94 (s, 1H), 8.13 (s, 1H), 11.89 (br s, 1H); LR-MS ESI 322.27 (100%). HRMS (ESI+ve): For C₁₇H₂₀N₅Si requires 322.1488 found 322.1490.

### Example 104

### 5-(2-Aminopyridin-4-yl)-7-phenyl-1H-indazol-3-amine

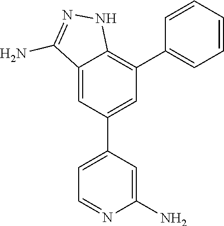 [SMILES: Nc1cc(-c2cc(-c3ccccc3)c3[nH]nc(N)c3c2)ccn1]

5-(2-Aminopyridin-4-yl)-7-chloro-3-amino-1H-indazole (Example 1) (0.050 g, 0.19 mmol), phenylboronic acid (0.046 g, 0.38 mmol) and [1,1′-bis(di-tert-butylphosphino)ferrocene]dichloropalladium(II) catalyst (0.006 g, 0.01 mmol) were placed in a 2-5 mL microwave vial which was sealed and purged with nitrogen. Ethanol (1.5 mL) was added and the suspension brought to 90° C. with stirring before addition of K₃PO₄ (O₂-free, 1 M, 0.60 mL). Stirring continued for 24 hours. Resultant solution was diluted in EtOAc, washed with water and adsorbed onto silica under reduced pressure. Chromatographic purification (Biotage SP4, 50 g cartridge, solvent system: EtOAc/methanol, 0-10%, 8 CV; 10%, 4 CV) yielded product as a pale yellow solid (0.024 g, 42%). ¹H (DMSO-d₆, 500 MHz) δ 5.53 (s, 2H), 5.87 (s, 2H), 6.79 (s, 1H), 6.87 (d, J=1.1 Hz, 1H), 7.46 (m, 1H), 7.53 (m, 3H), 7.74 (d, J=7.5 Hz, 1H), 7.95 (d, J=4.7 Hz, 1H) 8.08 (s, 1H), 11.65 (br s, 1H); LR-MS ESI 302.24 (100%). HRMS (ESI+ve): For C₁₈H₁₅N₅ requires 302.1406 found 302.1402.

### Example 105

### 5-(2-Aminopyridin-4-yl)-7-(3-fluorophenyl)-1H-indazol-3-amine

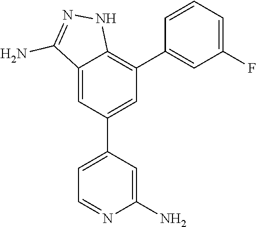 [SMILES: Nc1cc(-c2cc(-c3cccc(F)c3)c3[nH]nc(N)c3c2)ccn1]

This compound was prepared using an analogous procedure for Example 104 using 3-fluorophenylboronic acid (0.062 g, 0.38 mmol). Chromatographic purification (Biotage SP4, 50 g cartridge, solvent system: EtOAc/methanol, 0-10%, 8 CV; 10%, 4 CV) yielded product as off-white solid (0.017 g, 27%). ¹H (DMSO-d₆, 500 MHz) δ 5.58 (s, 2H), 5.87 (s, 2H), 6.79 (s, 1H), 6.89 (d, J=6.5 Hz, 1H), 7.26 (s, 1H), 7.59 (m, 2H), 7.95 (d, J=5.0 Hz, 1H), 8.12 (s, 1H), 11.74 (br s, 1H); LR-MS ESI 320.20 (100%). LRMS: For C₁₈H₁₄FN₅ requires 319.12 found 320.13.

### Example 106

### 5-(2-Aminopyridin-4-yl)-7-(3-(trifluoromethyl)phenyl)-1H-indazol-3-amine

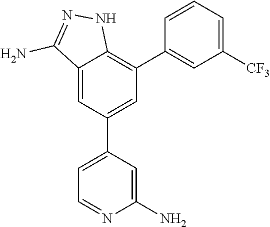 [SMILES: Nc1cc(-c2cc(-c3cccc(C(F)(F)F)c3)c3[nH]nc(N)c3c2)ccn1]

This compound was prepared using an analogous procedure for Example 104 using 3-trifluoromethyl phenylboronic acid (0.072 g, 0.38 mmol). Chromatographic purification (Biotage SP4, 50 g cartridge, solvent system: EtOAc/methanol, 0-10%, 8 CV; 10%, 4 CV) yielded product as an off-white solid (0.023 g, 33%); ¹H (DMSO-d₆, 500 MHz) δ 5.60 (s, 2H), 5.94 (s, 2H), 6.80 (s, 1H), 6.90 (dd, J=1.3 & 5.4 Hz, 1H), 7.60 (d, J=1.4 Hz, 1H), 7.76 (m, 2H), 7.96 (d, J=5.4 Hz, 1H) 8.08 (m, 2H), 8.14 (d, J=1.6 Hz, 1H) 11.80 (br s, 1H); LR-MS ESI 370.18 (100%). LRMS (ESI+ve): For C₁₉H₁₅F3N₅O requires 369.12 found 370.13.

### Example 107

### 7-(3-Aminophenyl)-5-(2-aminopyridin-4-yl)-1H-indazol-3-amine

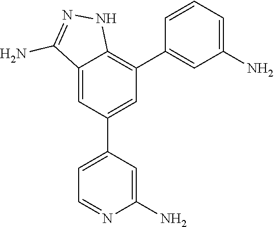 [SMILES: Nc1cccc(-c2cc(-c3ccnc(N)c3)cc3c(N)n[nH]c23)c1]

This compound was prepared using an analogous procedure for Example 104using 3-aminophenylboronic acid (0.082 g, 0.38 mmol). Chromatographic purification (Biotage SP4, 50 g cartridge, solvent system: EtOAc/methanol, 0-10%, 8 CV; 10%, 4 CV) yielded product as off-white solid (0.021 g, 35%). ¹H (DMSO-d₆, 500 MHz) 5.11 (s, 2H), 5.49 (s, 2H), 5.88 (s, 2H), 6.61 (d, J=5.2 Hz, 1H), 6.77 (s, 1H), 6.86 (m, 2H), 6.92 (s, 1H), 7.15 (t, J=5.3 Hz, 1H), 7.49 (s, 1H), 7.94 (d, J=5.4 Hz, 1H), 8.03 (s, 1H), 11.49 (br s, 1H); LR-MS ESI 317.23 (100%). LRMS: For C₁₈H₁₇N₆ requires 316.14 found 317.14

### Example 108

### 3-(3-Amino-5-(2-aminopyridin-4-yl)-1H-indazol-7-yl)phenol

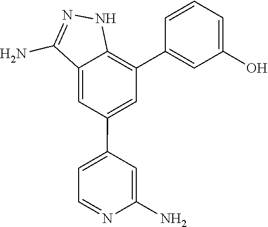 [SMILES: Nc1cc(-c2cc(-c3cccc(O)c3)c3[nH]nc(N)c3c2)ccn1]

This compound was prepared using an analogous procedure for Example 104Example 104 using 3-hydroxyphenylboronic acid (0.032 g, 0.23 mmol). Chromatographic purification (Biotage SP4, 50 g cartridge, solvent system: EtOAc/methanol, 0-10%, 8 CV; 10%, 4 CV) yielded product as off-white solid (0.010 g, 17%). ¹H (DMSO-d₆, 500 MHz) δ 5.51 (s, 2H), 5.88 (s, 2H), 6.80 (m, 3H), 7.09 (s, 1H), 7.15 (d, J=5.3 Hz, 1H), 7.30 (t, J=7.9 Hz, 1H) 7.49 (s, 1H), 7.96 (d, J=5.4 Hz, 1H), 8.06 (m, 1H), 11.59 (br s, 1H); LR-MS ESI 318.15 (100%). HRMS (ESI+ve): For C₁₈H₁₆N₅O requires 317.13 found 318.13.

### Example 109

### 5-(2-Aminopyridin-4-yl)-7-(3-methoxyphenyl)-1H-indazol-3-amine

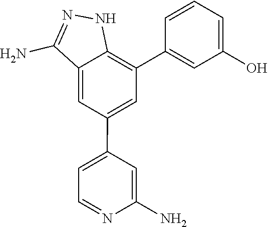 [SMILES: Nc1cc(-c2cc(-c3cccc(O)c3)c3[nH]nc(N)c3c2)ccn1]

This compound was prepared using an analogous procedure for Example 104 using 3-methoxyphenylboronic acid (0.057 g, 0.38 mmol). Chromatographic purification (Biotage SP4, 50 g cartridge, solvent system: EEtOAc/methanol, 0-10%, 8 CV; 10%, 4 CV) yielded product as an off-white solid (0.032 g, 51%); ¹H (DMSO-d₆, 500 MHz) δ 3.85 (s, 3H), 5.53 (s, 2H), 5.91 (s, 2H), 6.79 (s, 1H), 6.88 (dd, J=1.6 & 5.4 Hz, 1H), 6.88 (dd, J=2.2 & 7.8 Hz, 1H) 7.24 (s, 1H), 7.28 (d, J=7.5 Hz, 1H), 7.42 (t, J=7.9 Hz, 1H), 7.55 (d, J=1.6 Hz, 1H), 7.95 (d, J=5.7 Hz, 1H) 8.08 (d, J=1.6 Hz, 1H), 11.66 (br s, 1H); LR-MS ESI 332.21 (100%). HRMS (ESI+ve): For C₁₉H₁₈N₅O requires 332.14 found 332.15.

### Example 110

### 3-(3-Amino-5-(2-aminopyridin-4-yl)-1H-indazol-7-yl)phenyl)methanol

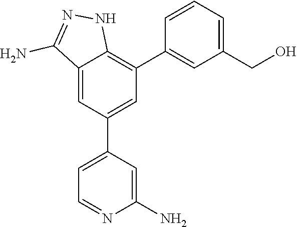 [SMILES: Nc1cc(-c2cc(-c3cccc(CO)c3)c3[nH]nc(N)c3c2)ccn1]

This compound was prepared using an analogous procedure for Example 104 using 3-hydroxymethylphenylboronic acid (0.072 g, 0.38 mmol). Chromatographic purification (Biotage SP4, 50 g cartridge, solvent system: EtOAc/methanol, 0-10%, 8 CV; 10%, 4 CV) yielded product as off-white solid (0.016 g, 25%); ¹H (DMSO-d₆, 500 MHz) δ 4.61 (d, J=6.0 Hz, 2H), 5.17 (t, J=1.6 Hz, 1H), 5.54 (s, 2H), 5.88 (s, 2H), 6.78 (s, 1H), 6.86 (dd, J=1.6 & 5.4 Hz, 1H), 7.38 (d, J=1.6 Hz, 1H), 7.42 (t, J=7.9 Hz, 1H), 7.56 (d, J=1.6 Hz, 1H), 7.59 (d, J=1.6 Hz, 1H), 7.65 (s, 1H), 7.94 (d, J=5.4 Hz, 1H), 8.07 (d, J=1.3 Hz, 1H), 11.61 (br s, 1H); LR-MS ESI 332.22 (100%). LRMS: For C₁₉H₁₈N₅O requires 331.14 found 332.14.

### Example 111

### 3-(3-Amino-5-(2-aminopyridin-4-yl)-1H-indazol-7-yl)benzaldehyde

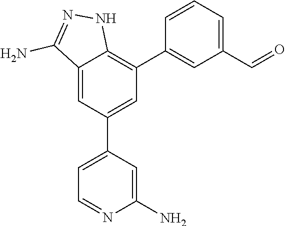 [SMILES: Nc1cc(-c2cc(-c3cccc(C=O)c3)c3[nH]nc(N)c3c2)ccn1]

This compound was prepared using an analogous procedure for Example 104 using 3-formylphenylboronic acid (0.072 g, 0.38 mmol). Chromatographic purification (Biotage SP4, 50 g cartridge, solvent system: EtOAc/methanol, 0-10%, 8 CV; 10%, 4 CV) yielded product as off-white solid (0.021 g, 32%). ¹H (DMSO-d₆, 500 MHz) δ 5.51 (s, 2H), 5.88 (s, 2H), 6.79 (s, 1H), 6.85 (m, 2H), 7.09 (s, 1H), 7.15 (d, J=7.9 Hz, 1H), 7.30 (t, J=7.9 Hz, 1H), 7.49 (d, J=1.6 Hz, 1H), 7.94 (d, J=5.4 Hz, 1H), 8.06 (d, J=1.3 Hz, 1H), 9.52 (s, 1H), 11.58 (br s, 1H); LRMS For C₁₉H₁₅N₅O requires 329.14 found 330.13.

### Example 112

### Ethyl 3-(3-amino-5-(2-aminopyridin-4-yl)-1H-indazol-7-yl)benzoate

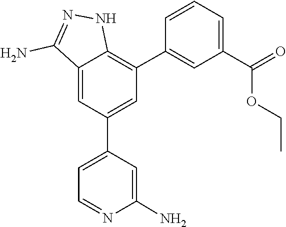 [SMILES: CCOC(=O)c1cccc(-c2cc(-c3ccnc(N)c3)cc3c(N)n[nH]c23)c1]

This compound was prepared using an analogous procedure for Example 104 using (3-(ethoxycarbonyl)phenyl)boronic acid (0.072 g, 0.38 mmol). Chromatographic purification (Biotage SP4, 50 g cartridge, solvent system: EtOAc/methanol, 0-10%, 8 CV; 10%, 4 CV) yielded product as off-white solid (0.024 g, 34%). ¹H (DMSO-d₆, 500 MHz) δ 1.34 (t, J=7.2 Hz, 3H), 4.36 (q, J=6.9 Hz, 2H), 5.62 (s, 2H), 5.89 (s, 2H), 6.79 (s, 1H), 6.88 (d, J=4.4 Hz, 1H), 7.56 (s, 1H), 7.68 (t, J=7.9 Hz, 1H), 7.99 (m, 3H), 8.13 (s, 1H), 8.23 (s, 1H), 11.74 (br s, 1H); LR-MS ESI 374.20 (100%). HRMS (ESI+ve): For C₂₁H₂₀N₅O₂ requires 373.15 found 374.16.

### Example 113

### 3-(3-Amino-5-(2-aminopyridin-4-yl)-1H-indazol-7-yl)benzamide

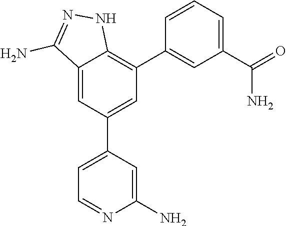 [SMILES: NC(=O)c1cccc(-c2cc(-c3ccnc(N)c3)cc3c(N)n[nH]c23)c1]

This compound was prepared using an analogous procedure for Example 104 using (3-carbamoylphenyl)boronic acid (0.075 g, 0.38 mmol). Chromatographic purification (Biotage SP4, 50 g cartridge, solvent system: EtOAc/methanol, 0-10%, 8 CV; 10%, 4 CV) yielded product as off-white solid (0.011 g, 16%). ¹H (DMSO-d₆, 500 MHz) δ 5.57 (s, 2H), 5.89 (s, 2H), 6.79 (s, 1H), 6.88 (d, J=1.9 & 6.9 Hz, 1H), 7.42 (s, 2H), 7.61 (m, 2H), 7.87 (t, J=7.5 Hz, 2H), 7.96 (d, J=5.3 Hz, 1H), 7.98 (m, 2H), 8.11 (m, 3H), 11.73 (br s, 1H); LRMS: For C₁₉H₁₆N₆O requires 344.14 found 345.14.

### Example 114

### 3-(3-Amino-5-(2-aminopyridin-4-yl)-1H-indazol-7-yl)benzenesulfonamide

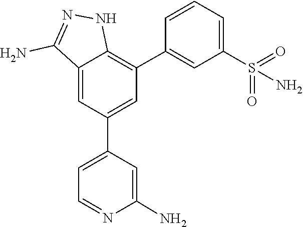 [SMILES: Nc1cc(-c2cc(-c3cccc(S(N)(=O)=O)c3)c3[nH]nc(N)c3c2)ccn1]

This compound was prepared using an analogous procedure for Example 104 using (3-sulfamoylphenyl)boronic acid (0.076 g, 0.38 mmol). Chromatographic purification (Biotage SP4, 50 g cartridge, solvent system: EtOAc/methanol, 0-10%, 8 CV; 10%, 4 CV) yielded product as off-white solid (0.023 g, 24%). ¹H (DMSO-d₆, 500 MHz) δ 5.66 (s, 2H), 5.94 (s, 2H), 6.78 (s, 1H), 6.88 (d, J=1.9 & 6.9 Hz, 1H), 7.38 (s, 2H), 7.61 (d, J=1.9 Hz, 1H), 7.73 (t, J=9.8 Hz, 1H), 7.87 (d, J=10.3 Hz, 1H), 7.98 (m, 2H), 8.16 (m, 2H), 11.70 (br s, 1H); LR-MS ESI 381.16 (100%). HRMS (ESI+ve): For C₁₈H₁₇N₆O₂S requires 381.1134 found 381.1145.

### Example 115

### 5-(2-Aminopyridin-4-yl)-7-(4-(methylsulfonyl)phenyl)-1H-indazol-3-amine

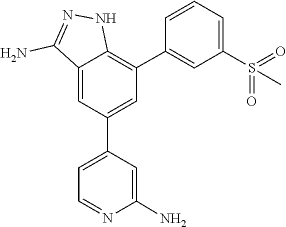 [SMILES: CS(=O)(=O)c1cccc(-c2cc(-c3ccnc(N)c3)cc3c(N)n[nH]c23)c1]

This compound was prepared using an analogous procedure for Example 104 using (3-(methylsulfonyl)phenyl)boronic acid (0.074 g, 0.38 mmol). Chromatographic purification (Biotage SP4, 50 g cartridge, solvent system: EtOAc/methanol, 0-10%, 8 CV; 10%, 4 CV) yielded product as off-white solid (0.036 g, 32%). ¹H (DMSO-d₆, 500 MHz) δ 3.33 (s, 3H), 5.62 (s, 2H), 5.89 (s, 2H), 6.78 (s, 1H), 6.89 (dd, J=1.6 & 5.4 Hz, 1H), 7.63 (d, J=1.3 Hz, 1H), 7.79 (t, J=7.9 Hz, 1H), 7.96 (d, J=5.7 Hz, 1H), 8.07 (d, J=8.2 Hz, 1H), 8.18 (s, 1H), 8.19 (s, 1H), 11.82 (br s, 1H); LR-MS ESI 380.13 (100%). HRMS (ESI+ve): For C₁₉H₁₈N₆OS requires 380.1181 found 380.1201.

### Example 116

### 5-(2-Aminopyridin-4-yl)-7-(3-morpholinomethyl)phenyl)-1H-indazol-3-amine

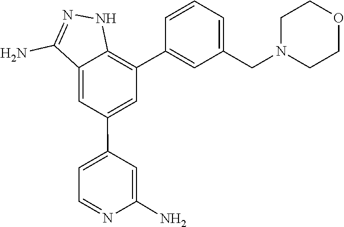 [SMILES: Nc1cc(-c2cc(-c3cccc(CN4CCOCC4)c3)c3[nH]nc(N)c3c2)ccn1]

This compound was prepare using an analogous procedure or Example 104 using (3-(morpholinomethyl)phenyl)boronic acid(0.051 g, 0.23 mmol). Chromatographic purification (Biotage SP4, 50 g cartridge, solvent system: EtOAc/methanol, 0-10%, 8 CV; 10%, 4 CV) yielded product as off-white solid (0.017 g, 34%). ¹H (DMSO-d₆, 500 MHz) δ 2.40 (m, 4H), 3.57 (m, 6H), 5.54 (s, 2H), 5.88 (s, 1H) 6.78 (s, 1H), 6.87 (d, J=5.5 Hz, 1H), 7.37 (d, J=7.9 Hz, 1H), 7.47 (t, J=7.9 Hz, 1H), 7.55 (s, J=1.6 Hz, 1H), 7.62 (m, 2H), 7.95 (d, J=5.4 Hz, 1H), 8.08 (d, J=1.6 Hz, 1H), 11.63 (br s, 1H); LR-MS ESI 401.22 (100%). HRMS (ESI+ve): For C₂₃H₂₅N₆O requires 401.2090 found 401.2096.

### Example 117

### 4-(3-Amino-5-(2-aminopyridin-4-yl)-1H-indazol-7-yl)phenol

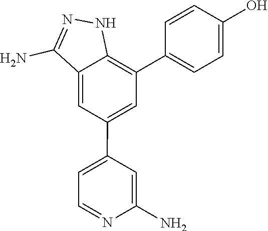 [SMILES: Nc1cc(-c2cc(-c3ccc(O)cc3)c3[nH]nc(N)c3c2)ccn1]

This compound was prepared using an analogous procedure for Example 104 using 4-hydroxyphenylboronic acid (0.032 g, 0.23 mmol). Chromatographic purification (Biotage SP4, 50 g cartridge, solvent system: EtOAc/methanol, 0-10%, 8 CV; 10%, 4 CV) yielded product as off-white solid (0.021 g, 39%). ¹H (DMSO-d₆, 500 MHz) δ 5.48 (s, 2H), 5.86 (s, 2H), 6.77 (s, 1H) 6.85 (d, J=5.4 Hz, 1H), 6.90 (d, J=8.5 Hz, 1H), 7.46 (s, 1H), 7.55 (d, J=8.5 Hz, 1H), 7.94 (d, J=5.4 Hz, 1H), 7.99 (m, 1H), 11.53 (br s, 1H); LR-MS ESI 318.15 (100%). HRMS (ESI+ve): For C₁₈H₁₆N₅O requires 317.1355 found 317.1358.

### Example 118

### 4-(3-Amino-5-(2-aminopyridin-4-yl)-1H-indazol-7-yl)phenyl)methanol

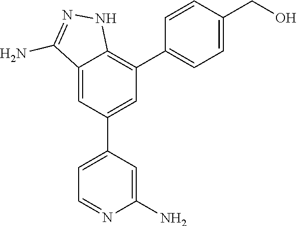 [SMILES: Nc1cc(-c2cc(-c3ccc(CO)cc3)c3[nH]nc(N)c3c2)ccn1]

This compound was prepared using an analogous procedure for Example 104 using 3-hydroxymethylphenylboronic acid (0.072 g, 0.38 mmol). Chromatographic purification (Biotage SP4, 50 g cartridge, solvent system: EtOAc/methanol, 0-10%, 8 CV; 10%, 4 CV) yielded product as off-white solid (0.012 g, 22%). ¹H (DMSO-d₆, 500 MHz) δ 4.59 (d, J=6.3 Hz, 2H), 5.29 (br s, 1H), 5.56 (s, 2H), 5.91 (s, 2H), 6.79 (s, 1H), 6.88 (d, J=6.0 Hz, 1H), 7.48 (d, J=9.4 Hz, 2H), 7.56 (s, 1H), 7.71 (d, J=9.4 Hz, 1H), 7.97 (d, J=6.6 Hz, 1H), 8.08 (s, 1H), 11.67 (br s, 1H); LR-MS ESI 332.22 (100%). HRMS (ESI+ve): For C₁₉H₁₈N₅O requires 332.1511 found 332.1523.

### Example 119

### 5-(2-Aminopyridin-4-yl)-7-(4-(dimethylamino)phenyl)-1H-indazol-3-amine

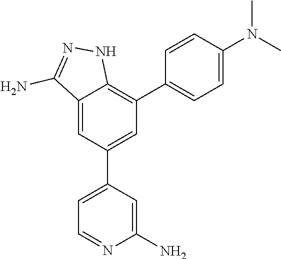 [SMILES: CN(C)c1ccc(-c2cc(-c3ccnc(N)c3)cc3c(N)n[nH]c23)cc1]

This compound was prepared using an analogous procedure for Example 104 using (4-(dimethylamino)phenyl)boronic acid (0.064 g, 0.38 mmol). Chromatographic purification (Biotage SP4, 50 g cartridge, solvent system: EtOAc/methanol, 0-10%, 8 CV; 10%, 4 CV) yielded product as off-white solid (0.016 g, 25%). ¹H (DMSO-d₆, 500 MHz) δ 2.98 (s, 6H), 5.51 (s, 2H), 5.97 (s, 2H), 6.80 (s, 1H), 6.89 (m, 3H), 7.58 (s, 1H), 7.60 (d, J=11.0 Hz, 2H), 7.96 (d, J=7.0 Hz, 1H), 8.99 (d, J=1.9 Hz, 1H), 11.56 (br s, 1H); LR-MS ESI 345.26 (100%). HRMS (ESI+ve): For C₂₀H₂₁N₆ requires 345.1828 found 345.1840.

### Example 120

### 4-(3-Amino-5-(2-aminopyridin-4-yl)-1H-indazol-7-yl)benzamide

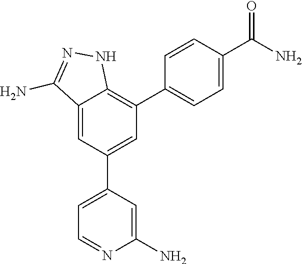 [SMILES: NC(=O)c1ccc(-c2cc(-c3ccnc(N)c3)cc3c(N)n[nH]c23)cc1]

This compound was prepared using an analogous procedure for Example 104 using (4-carbamoylphenyl)boronic acid (0.062 g, 0.38 mmol). Chromatographic purification (Biotage SP4, 50 g cartridge, solvent system: EtOAc/methanol, 0-10%, 8 CV; 10%, 4 CV) yielded product as off-white solid (0.013 g, 21%). ¹H (DMSO-d₆, 500 MHz) δ 5.58 (s, 2H), 5.95 (s, 2H), 6.81 (s, 1H), 6.90 (d, J=4.1 Hz, 1H), 7.40 (s, 1H), 7.62 (s, 1H), 7.82 (d, J=8.2 Hz, 1H), 7.96 (d, J=5.3 Hz, 1H), 8.03 (m, 3H), 8.12 (s, 1H), 11.72 (br s, 1H); LR-MS ESI 345.17 (100%). HRMS (ESI+ve): For C₁₉H₁₇N₆O requires 345.1464 found 345.1466.

### Example 121

### 4-(3-Amino-5-(2-aminopyridin-4-yl)-1H-indazol-7-yl)benzenesulfonamide

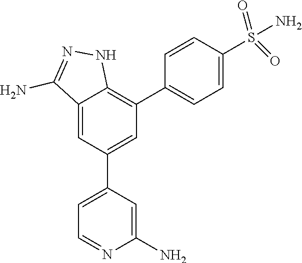 [SMILES: Nc1cc(-c2cc(-c3ccc(S(N)(=O)=O)cc3)c3[nH]nc(N)c3c2)ccn1]

This compound was prepared using an analogous procedure for Example 104 using (4-sulfamoylphenyl)boronic acid(0.076 g, 0.38 mmol). Chromatographic purification (Biotage SP4, 50 g cartridge, solvent system: EtOAc/methanol, 0-10%, 8 CV; 10%, 4 CV) yielded product as off-white solid (0.011 g, 13%). ¹H (DMSO-d₆, 500 MHz) δ 5.60 (s, 2H), 5.89 (s, 2H), 6.79 (s, 1H), 6.89 (d, J=4.1 Hz, 1H), 7.45 (s, 2H), 7.63 (s, 1H), 7.95 (m, 5H), 8.14 (s, 1H), 11.77 (br s, 1H); LR-MS ESI 381.30 (100%). HRMS (ESI+ve): For C₁₈H₁₇N₆O₂S requires 381.1134 found 381.1145.

### Example 122

### 5-(2-Aminopyridin-4-yl)-7-(4-(morpholinomethyl)phenyl)-1H-indazol-3-amine

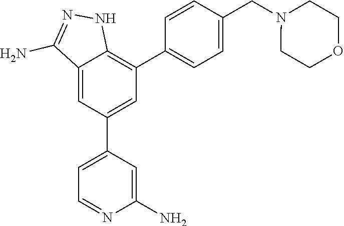 [SMILES: Nc1cc(-c2cc(-c3ccc(CN4CCOCC4)cc3)c3[nH]nc(N)c3c2)ccn1]

This compound was prepared using an analogous procedure for Example 104 using (4-(morpholinomethyl)phenyl)boronic acid (0.051 g, 0.23 mmol). Chromatographic purification (Biotage SP4, 50 g cartridge, solvent system: EtOAc/methanol, 0-10%, 8 CV; 10%, 4 CV) yielded product as off-white solid (0.020 g, 21%). ¹H (DMSO-d₆, 500 MHz) δ 2.41 (m, 4H), 3.54 (s, 2H), 3.60 (m, 4H), 5.53 (s, 2H), 5.93 (s, 2H), 6.80 (s, 1H) 6.88 (d, J=5.7 Hz, 1H), 7.45 (d, J=7.9 Hz, 1H), 7.55 (d, J=1.6 Hz, 1H), 7.70 (d, J=7.9 Hz, 2H), 7.94 (d, J=5.7 Hz, 1H), 8.08 (d, J=1.6 Hz, 1H), 11.63 (br s, 1H); LR-MS ESI 401.22 (100%). HRMS (ESI+ve): For C₂₃H₂₅N₆O requires 401.2090 found 401.2096.

### Example 123

### tert-Butyl (4-iodopyridin-2-yl)carbamate

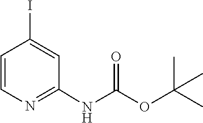

To a solution of 4-iodopyridin-2-amine (2.7 g 12.27 mmol) in dioxane (10 mL) was added DMAP (1.79 g, 14.72 mmol) under a blanket of argon followed by Boc₂O (2.812 g, 12.88 mmol). The resulting reaction mixture was stirred at 80° C. for 12 hours. The reaction was then cooled and quenched by pouring into water 20 mL, extracted with EtOAc (3×30 mL) and washed with brine (10 mL). The combined organic layers were concentrated and purified by flash eluting with a gradient of EtOAc:Petroleum Ether (0-50%) to afford the target compound as an off white solid (2.94 g, 75%). ¹H NMR (500 MHz, DMSO-d₆) δ 9.94 (s, 1H), 8.24 (d, J=1.4 Hz, 1H), 7.97 (d, J=5.2 Hz, 1H), 7.42 (dd, J=5.2, 1.5 Hz, 1H), 1.48 (s, 9H). LRMS (ESI+ve): C₁₀H₁₃IN₂O₂ Molecular Weight: 320.1305 found 321.0

### tert-Butyl (4-(3-amino-7-bromo-1H-indazol-5-yl)pyridin-2-yl)carbamate

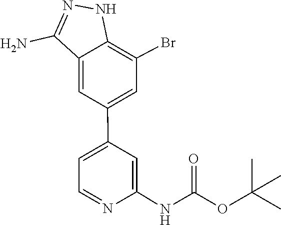 [SMILES: CC(C)(C)OC(=O)Nc1cc(-c2cc(Br)c3[nH]nc(N)c3c2)ccn1]

To a solution of tert-butyl (4-iodopyridin-2-yl)carbamate (1.3 g, 3.64 mmol) in a degassed solution of dioxane:H₂O (18:2 5 mL) was added cesium carbonate (3.556 g, 10.9 mmol), 7-bromo-5-(4,4,5,5-tetramethyl-1,3,2-dioxaborolan-2-yl)-1H-indazol-3-amine (1.28 g, 3.818 mmol) and bis(triphenylphosphine)palladium(II) dichloride catalyst (0.143 g, 0.204 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a light brown solid (1.05 g, 64%). ¹H NMR (500 MHz, DMSO-d₆) δ 11.99 (s, 1H), 9.79 (s, 1H), 8.28 (d, J=5.3 Hz, 1H), 8.19 (d, J=1.4 Hz, 1H), 8.09 (d, J=1.5 Hz, 1H), 7.79 (d, J=1.5 Hz, 1H), 7.33 (dd, J=5.3, 1.7 Hz, 1H), 5.72 (s, 2H), 1.51 (s, 9H). LRMS (ESI+ve): For C₁₇H₁₈BrN₅O₂ Molecular Weight: 404.2680 found 404.0, 405.0.

### 5-(2-Aminopyridin-4-yl)-7-(4-(tert-butyl)phenyl)-1H-indazol-3-amine

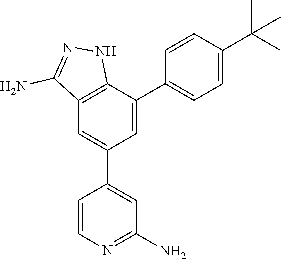 [SMILES: CC(C)(C)c1ccc(-c2cc(-c3ccnc(N)c3)cc3c(N)n[nH]c23)cc1]

To a solution of tert-butyl (4-(3-amino-7-bromo-1H-indazol-5-yl)pyridin-2-yl)carbamate (0.095 g, 0.235 mmol) and 4-tert-butylphenylboronic acid (0.050 g, 0.282 mmol) in a degassed solution of dioxane:H₂O (18:2 5 mL) was added sodium carbonate (0.075 g, 0.705 mmol), and dichloro[1, 1′-bis(di-tert-butylphosphino)ferrocene]palladium(II) catalyst (0.0082 g, 0.012 mmol) under an atmosphere of argon. The reaction was then sealed and stirred at 90° C. for 18 hours. The cooled reaction was then treated with TBAF to deprotect, diluted with EtOAc (30 mL) and washed with water (30 mL) and brine (10 mL). The organic layer was then concentrated under reduced pressure and purified by HPLC to give the desired compound as a yellow powder (0.012 g, 11%). ¹H NMR (500 MHz, DMSO-d₆) δ 8.38 (d, J=1.7 Hz, 1H), 8.11-8.06 (m, 1H), 8.01 (d, J=6.2 Hz, 2H), 7.78-7.75 (m, 2H), 7.73 (d, J=1.7 Hz, 1H), 7.66-7.60 (m, 2H), 7.39-7.36 (m, 2H), 1.42 (s, 9H). LRMS (ESI+ve): For C₂₂H₂₃N₅ Molecular weight 357.4610 found 358.2.

### Example 124

### 5-(2-Aminopyridin-4-yl)-7-(2-chlorophenyl)-1H-indazol-3-amine

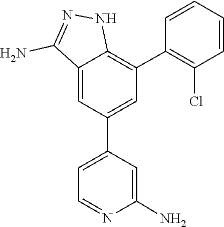 [SMILES: Nc1cc(-c2cc(-c3ccccc3Cl)c3[nH]nc(N)c3c2)ccn1]

This compound was prepared using an analogous procedure for Example 104 using 2-chlorophenylboronic acid (0.060 g, 0.38 mmol). Chromatographic purification (Biotage SP4, 50 g cartridge, solvent system: EtOAc/methanol, 0-10%, 8 CV; 10%, 4 CV) yielded product as off-white solid (0.028 g, 25%). ¹H (DMSO-d₆, 500 MHz) δ 5.50 (s, 2H), 5.87 (s, 2H), 6.75 (s, 1H), 6.83 (d, J=4.0 Hz, 1H), 7.45 (m, 2H), 7.51 (m, 1H), 7.60 (m, 1H), 7.95 (d, J=5.3 Hz, 1H), 8.13 (s, 1H), 11.43 (br s, 1H); LR-MS ESI 336.22 (100%), 338.22 (35%). HRMS (ESI+ve): For C₁₈H₁₅ClN₅ requires 336.1016 found 336.1032.

### Example 125

### 2-(3-Amino-5-(2-aminopyridin-4-yl)-1H-indazol-7-yl)phenyl)methanol

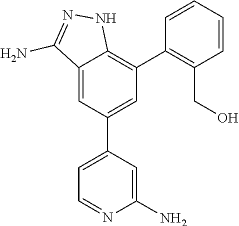 [SMILES: Nc1cc(-c2cc(-c3ccccc3CO)c3[nH]nc(N)c3c2)ccn1]

This compound was prepared using an analogous procedure for Example 104 using 2-hydroxymethylphenylboronic acid (0.072 g, 0.38 mmol). Chromatographic purification (Biotage SP4, 50 g cartridge, solvent system: EtOAc/methanol, 0-10%, 8 CV; 10%, 4 CV) yielded product as off-white solid (0.017 g, 14%). ¹H (DMSO-d₆, 500 MHz) δ 4.34 (d, J=5.1 Hz, 2H), 5.08 (t, J=5.0 Hz, 1H), 5.49 (s, 2H), 5.85 (s, 2H), 6.75 (s, 1H), 6.83 (dd, J=1.6 & 5.4 Hz, 1H), 7.36 (m, 3H), 7.45 (t, J=7.6 Hz, 1H), 7.65 (d, J=7.9 Hz, 1H), 7.93 (d, J=5.4 Hz, 1H), 8.07 (d, J=1.3 Hz, 1H), 11.61 (br s, 1H); LR-MS ESI 332.22 (100%). HRMS (ESI+ve): For C₁₉H₁₈N₅O requires 332.1511 found 332.1523.

### Example 126

### 4-(3-Amino-5-(2-aminopyridin-4-yl)-1H-indazol-7-yl)-3-methylbenzenesulfonamide

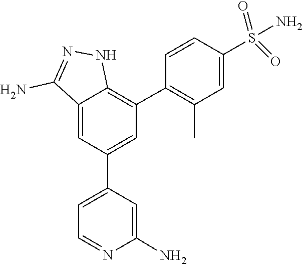 [SMILES: Cc1cc(S(N)(=O)=O)ccc1-c1cc(-c2ccnc(N)c2)cc2c(N)n[nH]c12]

This compound was prepared using an analogous procedure for Example 104 using (2-methyl-4-sulfamoylphenyl)boronic acid(0.074 g, 0.38 mmol). Chromatographic purification (Biotage SP4, 50 g cartridge, solvent system: EtOAc/methanol, 0-10%, 8 CV; 10%, 4 CV) yielded product as off-white solid (0.024 g, 16%). ¹H (DMSO-d₆, 500 MHz) δ 5.52 (s, 2H), 2.22 (s, 3H), 5.85 (s, 2H), 6.76 (s, 1H), 6.84 (s, 1H), 7.33 (s, 1H), 7.40 (s, 2H), 7.51 (d, J=7.8 Hz, 1H), 7.74 (d, J=7.9 Hz, 1H), 7.81 (s, 1H), 7.94 (m, 2H), 8.13 (s, 1H), 11.44 (br s, 1H); LR-MS ESI 395.26 (100%). HRMS (ESI+ve): For C₁₉H₁₉N₆O₂S requires 395.1290 found 395.1290.

### Example 127

### 5-(2-Aminopyridin-4-yl)-7-(pyridin-4-yl)-1H-indazol-3-amine

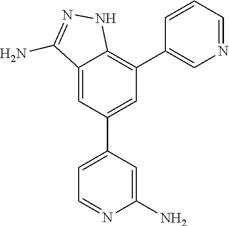 [SMILES: Nc1cc(-c2cc(-c3cccnc3)c3[nH]nc(N)c3c2)ccn1]

This compound was prepared using an analogous procedure for Example 104 using pyridin-3-ylboronic acid (0.046 g, 0.38 mmol). Chromatographic purification (Biotage SP4, 50 g cartridge, solvent system: EtOAc/methanol, 0-10%, 8 CV; 10%, 4 CV) yielded product as off-white solid (0.031 g, 27%). ¹H (DMSO-d₆, 500 MHz) δ 5.59 (s, 2H), 5.89 (s, 2H), 6.81 (s, 1H), 6.90 (d, J=4.4 Hz, 1H), 7.54 (m, 1H), 7.61 (s, 1H), 7.96 (d, J=4.7 Hz, 1H), 8.14 (s, 1H), 8.62 (d, J=3.8 Hz, 1H), 11.81 (br s, 1H); LR-MS ESI 303.10 (100%). HRMS (ESI+ve): For C₁₇H₁₅N₆ requires 303.1358 found 303.1346.

### Example 128

### 5-(2-Aminopyridin-4-yl)-7-(pyridin-4-yl)-1H-indazol-3-amine

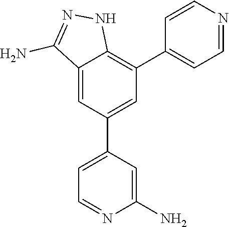 [SMILES: Nc1cc(-c2cc(-c3ccncc3)c3[nH]nc(N)c3c2)ccn1]

This compound was prepared using an analogous procedure for Example 104 using pyridin-4-ylboronic acid (0.075 g, 0.38 mmol). Chromatographic purification (Biotage SP4, 50 g cartridge, solvent system: EtOAc/methanol, 0-10%, 8 CV; 10%, 4 CV) yielded product as off-white solid (0.010 g, 11%). ¹H (DMSO-d₆, 500 MHz) δ 5.63 (s, 2H), 5.91 (s, 2H), 6.80 (s, 1H), 6.88 (d, 4.5 Hz, 1H), 7.69 (s, 1H), 7.76 (s, 1H), 7.96 (d, J=4.8 Hz, 1H), 8.19 (s, 1H), 8.68 (d, J=3.9 Hz, 1H), 11.82 (br s, 1H); LR-MS ESI 303.10 (100%). HRMS (ESI+ve): For C₁₇H₁₅N₆ requires 303.1358 found 303.1346.

### Example 129

### 5-(2-Aminopyridin-4-yl)-7-(furan-3-yl)-1H-indazol-3-amine

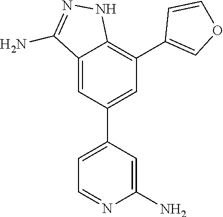 [SMILES: Nc1cc(-c2cc(-c3ccoc3)c3[nH]nc(N)c3c2)ccn1]

This compound was prepared using an analogous procedure for Example 104 using furan-3-ylboronic acid (0.056 g, 0.38 mmol). Chromatographic purification (Biotage SP4, 50 g cartridge, solvent system: EtOAc/methanol, 0-10%, 8 CV; 10%, 4 CV) yielded product as off-white solid (0.022 g, 25%). ¹H (DMSO-d₆, 500 MHz) 5.58 (s, 2H), 5.87 (s, 2H), 6.78 (s, 1H), 6.87 (dd, J=1.6 & 5.4 Hz, 1H), 7.19 (s, 1H), 7.73 (s, 1H), 7.82 (s, 1H), 7.96 (d, J=5.0 Hz, 1H), 8.01 (s, 1H), 8.40 (s, 1H), 11.48 (br s, 1H); LR-MS ESI 292.20 (100%). HRMS (ESI+ve): For C₁₆H₁₄N₅O requires 292.1198 found 292.1203.

### Example 130

### 5-(2-Aminopyridin-4-yl)-7-(thiophen-3-yl)-1H-indazol-3-amine

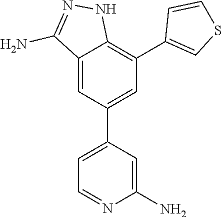 [SMILES: Nc1cc(-c2cc(-c3ccsc3)c3[nH]nc(N)c3c2)ccn1]

This compound was prepared using an analogous procedure for Example 104 using thiophen-3-ylboronic acid (0.058 g, 0.38 mmol). Chromatographic purification (Biotage SP4, 50 g cartridge, solvent system: EtOAc/methanol, 0-10%, 8 CV; 10%, 4 CV) yielded product as off-white solid (0.042 g, 48%). ¹H (DMSO-d₆, 500 MHz) 5.57 (s, 2H), 5.89 (s, 2H), 6.79 (s, 1H), 6.89 (d, J=5.4 Hz, 1H), 7.70 (m, 3H), 7.95 (d, J=5.0 Hz, 1H), 7.99 (s, 1H), 8.05 (s, 1H), 11.6 (br s, 1H); LR-MS ESI 308.22 (100%). HRMS (ESI+ve): For C₁₅H₁₄N₅S requires 308.0970 found 308.0972.

### Example 131

### 5-(2-Aminopyridin-4-yl)-7-(thiophen-2-yl)-1H-indazol-3-amine

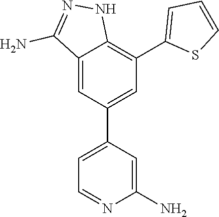 [SMILES: Nc1cc(-c2cc(-c3cccs3)c3[nH]nc(N)c3c2)ccn1]

This compound was prepared using an analogous procedure for Example 104 using thiophen-2-ylboronic acid (0.058 g, 0.38 mmol). HLPC purification (see general experimental section) yielded product as off-white solid (0.031 g, 27%). ¹H (DMSO-d₆, 500 MHz) 7.25 (m, 3H), 7.70 (m, 3H), 7.89 (s, 2H), 8.02 (d, J=5.0 Hz, 1H), 8.30 (s, 1H), 11.95 (br s, 1H), 13.23 (br s, 1H); LR-MS ESI 308.27 (100%). HRMS (ESI+ve): For C₁₅H₁₄N₅S requires 308.0970 found 308.0972.

### Example 132

### 5-(2-Aminopyridin-4-yl)-7-(thiazol-5yl)-1H-indazol-3-amine

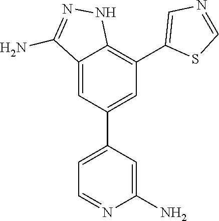 [SMILES: Nc1cc(-c2cc(-c3cncs3)c3[nH]nc(N)c3c2)ccn1]

This compound was prepared using an analogous procedure for Example 104 using thiazol-5-ylboronic acid (0.049 g, 0.38 mmol). Chromatographic purification (Biotage SP4, 50 g cartridge, solvent system: EtOAc/methanol, 0-10%, 8 CV; 10%, 4 CV) yielded product as off-white solid (0.034 g, 27%). ¹H (DMSO-d₆, 500 MHz) 5.72 (s, 2H), 5.96 (s, 2H), 6.76 (s, 1H), 6.85 (d, J=3.8 Hz, 1H), 7.67 (s, 1H), 7.98 (d, J=5.5 Hz, 1H), 8.15 (s, 1H), 8.47 (s, 1H), 9.20 (s, 1H), 11.81 (br s, 1H); LR-MS ESI 309.21 (100%). HRMS (ESI+ve): For C₁₅H₁₃N₆S requires 309.0922 found 309.0972.

### Example 133

### 5-(2-Aminopyridin-4-yl)-7-(1H-pyrazol-5-yl)-1H-indazol-3-amine

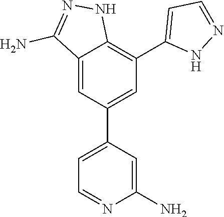 [SMILES: Nc1cc(-c2cc(-c3ccn[nH]3)c3[nH]nc(N)c3c2)ccn1]

A 2-5 mL MW tube was charged with a mixture of 5-(2-aminopyridin-4-yl)-7-chloro-1H-indazol-3-amine (Example 1) (52 mg, 0.2 mmol, 1 eq.), potassium trifluoro(1H-pyrazol-5-yl)borate (52 mg, 0.2 mmol, 1.5 mmol, 1.5 eq.), XphosG2 (7.9 mg, 0.01 mmol, 5 mol %) in 1,4-dioxane (oxygen-free, 1.8 mL) was heated to 60° C. under a gentle flow of nitrogen for 5 minutes, then 1 M aq. K₂CO₃ (oxygen-free, 0.6 mL, 1 mmol, 3 eq.) was added and the reaction mixture was heated to 110° C. for 15 hours. The reaction mixture was then cooled to room temperature, diluted with water (50 mL) and 1 M aq. K₂CO₃ (1 mL), and extracted with EtOAc (80 mL). The organic layer was separated and washed with water (50 mL), dried (MgSO₄) and purified by flash chromatography to give the title compound as a yellow solid (27 mg).

¹H NMR (400 MHz, DMSO-D₆) δ 5.57 (s, 2H), 5.92 (s, 2H), 6.79 (s, 1H), 6.88 (d, J=5.3 Hz, 1H), 7.01 (d, J=2.2 Hz, 1H), 7.88 (s, 1H), 7.91-8.00 (m, 2H), 8.04 (s, 1H), 11.11 (s, 1H), 13.09 (s, 1H).

### Example 134

### 5-(2-Aminopyridin-4-yl)-7-(3-methylbut-1-yn-1-yl)-1H-indazol-3-amine

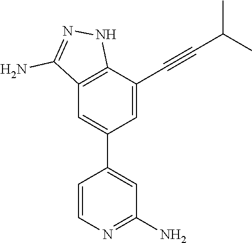 [SMILES: CC(C)C#Cc1cc(-c2ccnc(N)c2)cc2c(N)n[nH]c12]

In a sealed tube, a solution of 5-(2-aminopyridin-4-yl)-7-bromo-1H-indazol-3-amine (0.03 g, 0.09 mmol), copper (I) iodide (0.0008 g, 0.0045 mmol) and [1,1′-bis(diphenylphosphino)ferrocene]dichloropalladium(II) catalyst (0.003 g, 0.0045 mmol) in 0.27 ml of tetrahydrofuran:triethylamine (1:4) was degassed followed by addition of 3-methylbut-1-yne (0.013 ml, 0.13 mmol). The reaction mixture was stirred at room temperature for 16 h. After the reaction was completed, the reaction mixture was diluted with EtOAc and extracted with water. Extracted organic layer was dried over magnesium sulfate and concentrated under reduced pressure. The crude product was then purified by HPLC to give the titled compound as yellow solid (15 mg, 57%). ¹H NMR (400 MHz, DMSO-d₆) δ ppm 1.30 (d, J=6.78 Hz, 6H) 2.90 (spt, J=6.86 Hz, 1H) 7.20-7.25 (m, 2H) 7.63 (d, J=1.51 Hz, 1H) 7.89 (br. s., 2H) 8.00 (d, J=7.53 Hz, 1H) 8.29 (d, J=1.51 Hz, 1H) 12.08 (br. s., 1H). m/z (ESI-HRMS) calculated for C₁₇H₂₈N₅=292.1484 found=292.1551.

### Example 135

### 4-Iodopyridin-2-amine

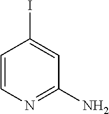

In a sealed 25 mL microwave vial, equipped with a magnetic stir bar, 2-fluoro-4-iodopyridine (1.5 g, 6.72 mmol, 1 Eq) was dissolved in 2 mL of Dioxane. 10 mL of ammonium hydroxide solution (28% in H₂O) was added, and the resulting reaction mixture was heated at 110° C. under microwave irradiation for 4 hours.

After cooling the reaction was filtered under vacuum and washed three times with water and petroleum ether to obtain white crystals of the titled compound (1.25 g, 5.712 mmol, 85%).

¹H NMR (500 MHz, DMSO-d₆) δ 7.61 (d, J=5.34 Hz, 1H), 6.86 (br. s., 1H) 6.81 (dd, J=5.3, 1.4 Hz, 1H), 6.07 (s, 2H) ppm.

### 5-(2-aminopyridin-4-yl)-7-bromo-1H-indazol-3-amine

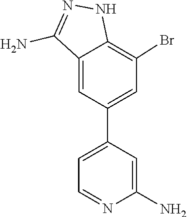 [SMILES: Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (200 mg, 0.59 mmol, 1.1 Eq) in a degassed 9:1 solution of dioxane/water (5 mL), were added 4-iodopyridin-2-amine (118.34 mg, 0.537 mmol, 1 Eq), cesium carbonate (524.89 g, 1.611 mmol, 3 Eq) and bis(triphenylphosphine)palladium chloride (37.69 mg, 0.0537 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography eluting with a gradient of EtOAc:Petroleum Ether (0-100%) to afford the titled compound (138.82 mg, 0.456 mmol, 85%). ¹H NMR (500 MHz, DMSO-d₆) δ 11.92 (br. s., 1H), 8.11 (s, 1H), 7.95 (d, J=5.3 Hz, 1H), 7.71 (s, 1H), 6.86-6.76 (m, 1H), 6.71 (s, 1H), 5.92 (s, 2H), 5.64 (br. s., 2H) ppm. LRMS (ESI+ve): For C₁₂H₁₀BrN₅ Molecular Weight: 304.15 found 304.1, 306.1, 307.0 (M+H).

### 5-(2-Aminopyridin-4-yl)-7-(pent-1-yn-1-yl)-1H-indazol-3-amine

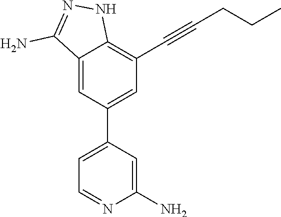 [SMILES: CCCC#Cc1cc(-c2ccnc(N)c2)cc2c(N)n[nH]c12]

To a solution of 5-(2-aminopyridin-4-yl)-7-bromo-1H-indazol-3-amine (130 mg, 0.427 mmol, 1 Eq) in a degassed 4:1 solution of DMF/triethylamine (5 mL) were added copper(l) iodide (16.26 mg, 0.0854 mmol, 0.2 Eq), [1,1′-bis(diphenylphosphino)ferrocene]dichloropalladium(II) catalyst (31.24 mg, 0.0427 mmol, 0.1 Eq) and 1-pentyne (43.63 mg, 0.063 mL, 0.64 mmol, 1.5 Eq), under an atmosphere of argon. The reaction was then sealed and heated at 70° C. for 18 hours.

The cooled reaction was diluted with EtOAc (20 mL) and washed with water (10 mL) and brine (2×10 mL). The organic layer was concentrated under reduced pressure and the crude was purified by flash column chromatography eluting with a gradient of EtOAc:Petroleum Ether (0-100% and 10% MeOH) followed by HPLC purification to give the titled product (111.96 mg, 0.384 mmol, 90%). ¹H NMR (500 MHz, CD₃OD) δ 8.05 (d, J=2.0 Hz, 1H), 7.90 (d, J=6.1 Hz, 1H), 7.66 (d, J=1.8 Hz, 1H), 7.04 (dd, J=6.0, 1.9 Hz, 1H), 6.98 (br s, 1H), 2.53 (t, J=7.1 Hz, 2H), 1.71 (sextet, J=7.2 Hz, 2H), 1.29 (br. s, 2H), 1.11 (t, J=7.4 Hz, 3H) ppm. LRMS: For C₁₇H₁₇N₅ requires 291.3 found 292.1 (M+H).

### Example 136

### tert-Butyl (4-iodopyridin-2-yl)carbamate

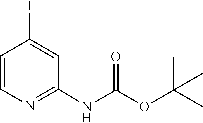

To a solution of 4-iodopyridin-2-amine (1 g, 4.54 mmol, 1 Eq) in dioxane (10 mL) was added DMAP (666.13 mg, 5.45 mmol, 1.2 Eq) under a blanket of argon followed by the addition of Boc₂O (1.04 g, 4.77 mmol, 1.05 Eq). The resulting reaction mixture was stirred at 80° C. overnight.

The reaction was then cooled and quenched by pouring into water (10 mL), extracted with EtOAc (3×15 mL) and washed with brine (10 mL). The combined organic layers were concentrated and purified by flash column chromatography eluting with a gradient of EtOAc:Petroleum Ether (0-50%) to afford the target compound as a white solid (1.162 g, 3.632 mmol, 80%). ¹H NMR (500 MHz, DMSO-d₆) δ 9.94 (s, 1H), 8.24 (d, J=1.4 Hz, 1H), 7.97 (d, J=5.2 Hz, 1H), 7.42 (dd, J=5.2, 1.5 Hz, 1H), 1.48 (s, 9H). LRMS (ESI+ve): C₁₀H₁₃IN₂O₂ Molecular Weight: 320.1305 found 321.0 (M+H).

### tert-butyl (4-(3-amino-7-bromo-1H-indazol-5-yl)pyridin-2-yl)carbamate

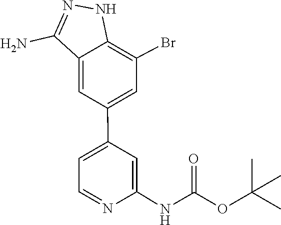 [SMILES: CC(C)(C)OC(=O)Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (1.353 g, 4.00 mmol, 1.1 Eq) in a degassed 9:1 solution of dioxane/water (10 mL), were added tert-butyl (4-iodopyridin-2-yl)carbamate (1.3 g, 3.64 mmol, 1 Eq), cesium carbonate (3.557 g, 10.92 mmol, 3 Eq) and bis(triphenylphosphine)palladium chloride (255.49 mg, 0.364 mmol, 0.1 Eq) under argon atmosphere. The reaction was then sealed and stirred at 70° C. overnight.

After cooling, the reaction was with EtOAc (30 mL) and washed with water (30 mL) and brine (10 mL). The organic layer was then concentrated under reduced pressure and purified by flash column chromatography eluting with a gradient of EtOAc:Petroleum Ether (50-100%) to afford the desired compound as an off white solid (941.78 mg, 2.329 mmol, 64%). ¹H NMR (500 MHz, DMSO-d₆) δ 11.99 (s, 1H), 9.79 (s, 1H), 8.28 (d, J=5.3 Hz, 1H), 8.19 (d, J=1.4 Hz, 1H), 8.09 (d, J=1.5 Hz, 1H), 7.79 (d, J=1.5 Hz, 1H), 7.33 (dd, J=5.3, 1.7 Hz, 1H), 5.72 (s, 2H), 1.51 (s, 9H). LRMS (ESI+ve): For C₁₇H₁₈BrN₅O₂ Molecular Weight: 404.2680 found 404.0, 405.0 (M+H).

### tert-Butyl (4-(3-amino-7-(cyclopropylethynyl)-1H-indazol-5-yl)pyridin-2-yl)carbamate

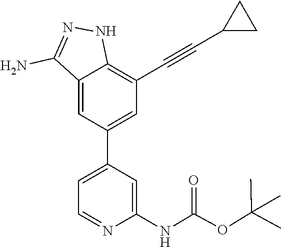 [SMILES: CC(C)(C)OC(=O)Nc1cc(-c2cc(C#CC3CC3)c3[nH]nc(N)c3c2)ccn1]

To a solution of tert-butyl (4-(3-amino-7-bromo-1H-indazol-5-yl)pyridin-2-yl)carbamate (230 mg, 0.568 mmol, 1 Eq) in a degassed 4:1 solution of DMF/triethylamine (5 mL) were added copper(I) iodide (21.63 mg, 0.113 mmol, 0.2 Eq), [1,1′-bis(diphenylphosphino)ferrocene]dichloropalladium(II) catalyst (41.56 mg, 0.056 mmol, 0.1 Eq) and ethynylcyclopropane (56.38 mg, 0.072 mL, 0.853 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography eluting with a gradient of EtOAc:Petroleum Ether (0-100%) to afford the titled product (88.48 mg, 0.227 mmol, 40%). ¹H NMR (500 MHz, DMSO-d₆) δ 11.86 (br. s., 1H), 9.76 (s, 1H), 8.26 (d, J=5.2 Hz, 1H), 8.12 (s, 1H), 8.08 (s, 1H), 7.51-7.57 (m, 2H), 7.30 (d, J=5.2 Hz, 1H), 5.62 (br. s., 2H), 1.65-1.59 (m, 1H), 1.50 (s, 9H), 0.94-0.90 (m, 4H) ppm.

### 5-(2-Aminopyridin-4-yl)-7-(cyclopropylethynyl)-1H-indazol-3-amine SU1674

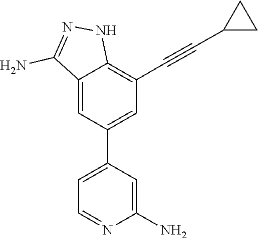 [SMILES: Nc1cc(-c2cc(C#CC3CC3)c3[nH]nc(N)c3c2)ccn1]

Trifluoroacetic acid (233.74 mg, 0.157 mL, 2.05 mmol, 20 Eq) was added to a solution of tert-butyl (4-(3-amino-7-(cyclopropylethynyl)-1H-indazol-5-yl)pyridin-2-yl)carbamate (80 mg, 0.205 mmol, 1 Eq) in DCM (2 mL) at 0° C. The reaction mixture was then allowed to stir at room temperature for 3 h. The reaction was quenched with saturated solution of NaHCO₃ (5 mL). The organic layer was washed with brine (2×5 mL), dried over anhydrous Na₂SO₄ and evaporated under reduced pressure. The crude residue was purified by flash column chromatography eluting with a gradient of EtOAc:Petroleum Ether (0-100%) to afford the titled product as a yellow solid (41.5 mg, 0.143 mmol, 70%). ¹H NMR (500 MHz, DMSO) δ 11.79 (s, 1H), 8.03 (d, J=1.7 Hz, 1H), 7.93 (d, J=5.3 Hz, 1H), 7.48 (d, J=1.7 Hz, 1H), 6.78 (dd, J=5.4, 1.8 Hz, 1H), 6.70 (d, J=2.0 Hz, 1H), 5.88 (s, 2H), 5.54 (s, 2H), 1.63-1.58 (m, 1H), 0.94-0.87 (m, 4H) ppm. LRMS (ESI+ve): For C₁₇H₁₅N₅ Molecular Weight: 289.34 found 290.1 (M+H).

### 5-(2-Aminopyridin-4-yl)-7-(3,3-dimethylbut-1-yn-1-yl)-1H-indazol-3-amine

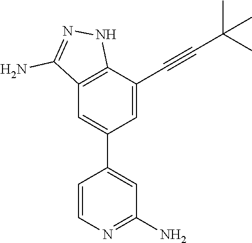 [SMILES: CC(C)(C)C#Cc1cc(-c2ccnc(N)c2)cc2c(N)n[nH]c12]

A 2-5 mL MW tube was charged with a mixture of 5-(2-aminopyridin-4-yl)-7-chloro-1H-indazol-3-amine (Example 1) (52 mg, 0.2 mmol, 1 eq.), (3,3-dimethylbut-1-yn-1-yl)trifluoroborate (75 mg, 0.4 mmol, 0.4 mmol, 2 eq.), XphosG2 (7.9 mg, 0.01 mmol, 5 mol %) in 1,4-dioxane (oxygen-free, 1.8 mL) was heated to 60° C. under a gentle flow of nitrogen for 5 minutes, then 2 M aq. K₂CO₃ (oxygen-free, 0.6 mL, 1.2 mmol, 6 eq.) was added and the reaction mixture was heated to 110° C. for 11 hours. The reaction mixture was then cooled to room temperature, diluted 1 M aq. K₂CO₃ (100 mL), and extracted with EtOAc (100 mL). The organic layer was separated and washed 1 M aq. K₂CO₃ (100 mL), dried (MgSO₄) and purified by flash chromatography (Silica, 50 g, 2-8% MeOH in AcOEt containing 1% triethylamine) to give the title compound as a yellow solid (46 mg, 75%). ¹H NMR (500 MHz, DMSO) δ 1.36 (s, 9H), 5.57 (s, 2H), 5.88 (s, 2H), 6.72 (s, 1H), 6.79 (dd, J=5.3, 1.6 Hz, 1H), 7.46 (d, J=1.4 Hz, 1H), 7.93 (d, J=5.4 Hz, 1H), 8.05-8.07 (m, 1H), 11.76 (s, 1H). HRMS: Calculated for C₁₈H₁₉N₅ 305.1640; Found: LRMS (ESI+ve): For C₁₈H₁₉N₅ Molecular weight 305.3850 found 306.3.

### Example 138

### 5-(2-Aminopyridin-4-yl)-7-(phenylethynyl)-1H-indazol-3-amine

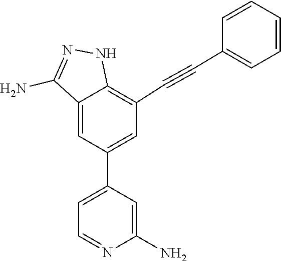 [SMILES: Nc1cc(-c2cc(C#Cc3ccccc3)c3[nH]nc(N)c3c2)ccn1]

5-(2-Aminopyridin-4-yl)-7-bromo-3-amino-1H-indazole (0.030 g, 0.1 mmol), phenylacetylene (37 μL, 0.34 mmol) and Tetrakis(triphenylphosphine)palladium(0) catalyst (0.012 g, 0.01 mmol) and CuI (0.002 g, 0.01 mmol) were placed in a 2-5 mL microwave vial which was sealed and purged with nitrogen. DMF (1.5 mL) and Net₃ (1.5 mL) were added and the suspension brought to 60° C. with stirring and maintained for 16 hrs. Resultant solution was diluted in EtOAc, washed with water and adsorbed onto silica under reduced pressure. Chromatographic purification (Biotage SP4, 50 g cartridge, solvent system: EEtOAc/methanol, 0-10%, 8 CV; 10%, 4 CV) yielded product as off-white solid (0.017 g, 27%). ¹H (DMSO-d₆, 500 MHz) 5.63 (s, 2H), 5.90 (s, 2H), 6.76 (s, 1H), 6.82 (d, J=3.8 Hz, 1H), 7.44 (m, 4H), 7.67 (s, 1H), 7.73 (d, J=5.5 Hz, 1H), 7.96 (s, 1H), 8.15 (s, 1H), 12.06 (br s, 1H); LR-MS ESI 326.33 (100%). HRMS (ESI+ve): For C₂₀H₁₆N₅ requires 326.1406 found 326.1406.

### Example 139

### 4-(3-Amino-5-(2-aminopyridin-4-yl)-1H-indazol-7-yl)but-3-yn-1-ol

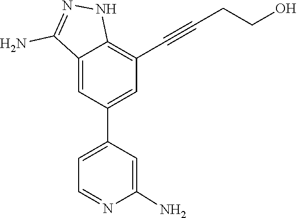 [SMILES: Nc1cc(-c2cc(C#CCCO)c3[nH]nc(N)c3c2)ccn1]

5-(2-Aminopyridin-4-yl)-7-bromo-3-amino-1H-indazole (0.030 g, 0.1 mmol), but-3-yn-1-ol (31 μL, 0.34 mmol) and Pd(PPh₃)₄(0.012 g, 0.01 mmol) and CuI (0.002 g, 0.01 mmol) were placed in a 2-5 mL microwave vial which was sealed and purged with nitrogen. DMF (1.5 mL) and Net₃ (1.5 mL) were added and the suspension brought to 60° C. with stirring and maintained for 16 hrs. Resultant solution was diluted in EtOAc, washed with water and adsorbed onto silica under reduced pressure. Chromatographic purification (Biotage SP4, 50 g cartridge, solvent system: EtOAc/methanol, 0-10%, 8 CV; 10%, 4 CV) yielded product as off-white solid (0.013 g, 44%). ¹H (DMSO-d₆, 500 MHz) 3.65 (m, 2H), 4.07 (m, 2H), 4.92 (m, 1H), 5.55 (s, 2H), 5.87 (s, 2H), 6.70 (s, 1H), 6.77 (d, J=3.8 Hz, 1H), 7.50 (s, 1H), 7.92 (s, 1H), 8.05 (s, 1H), 11.78 (br s, 1H); LR-MS ESI 294.27 (100%). HRMS (ESI+ve): For C₁₆H₁₆N₅O requires 294.1355 found 294.1355.

### Example 140

### 4-(3-Amino-5-(2-aminopyridin-4-yl)-1H-indazol-7-yl)-2-methylbut-3-yn-2-ol

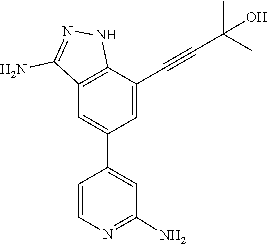 [SMILES: CC(C)(O)C#Cc1cc(-c2ccnc(N)c2)cc2c(N)n[nH]c12]

A 2-5 mL MW tube was charged with a mixture of 5-(2-aminopyridin-4-yl)-7-bromo-1H-indazol-3-amine (50 mg, 0.15 mmol, 1 eq.), 2-methylbut-3-yn-2-ol (30 uL, 0.3 mmol, 2 eq.), [1,1′-bis(diphenylphosphino)ferrocene]dichloropalladium(II) catalyst (16 mg mg, 0.02 mmol, 15 mol %) and CuI (4.2 mg 0.02 mmol, 15 mol %) in DMF (1 mL) and triethylamine (1 mL). The tube was then heated to 85° C. under nitrogen for 16 hrs. The reaction mixture was then cooled to room temperature, diluted with water (10 mL), the mixture was extracted with ethyl acetate (3×10 mL) and the organic fractions combined and dried over anhydrous magnesium sulfate, filtered and the solvent removed under high vacuum. The resulting residue was purified by HPLC to give the desired product as a yellow solid (22 mg, 0.05 mmol, 37%). ¹H NMR (500 MHz, DMSO-D₆) δ 1.54 (s, 6H), 5.42 (s, 1H), 5.61 (s, 2H), 5.90 (s, 2H), 6.72 (s, 1H), 6.80 (d, J=1.5 Hz, 1H) 7.50 (s, 1H), 7.95 (d, J=1.8 Hz, 1H), 8.10 (s, 1H), 11.76 (s, 1H). LRMS: Calculated for C₁₇H₁₇N₅O 307.14; Found: 308.20.

### Example 141

### 5-(2-Aminopyridin-4-yl)-7-((3-methyloxetan-3-yl)ethynyl)-1H-indazol-3-amine

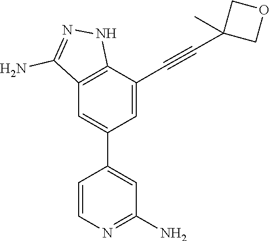 [SMILES: CC1(C#Cc2cc(-c3ccnc(N)c3)cc3c(N)n[nH]c23)COC1]

In a sealed tube, a solution of 5-(2-aminopyridin-4-yl)-7-bromo-1H-indazol-3-amine (0.05 g, 0.15 mmol), copper (I) iodide (0.0014 g, 0.0075 mmol) and [1,1′-bis(diphenylphosphino)ferrocene]dichloropalladium(II) catalyst (0.005 g, 0.0075 mmol) in 0.45 ml of tetrahydrofuran:triethylamine (1:4) was degassed followed by addition of 3-ethynyl-3-methyloxetane (0.02 ml, 0.22 mmol). The reaction mixture was stirred at room temperature for 16 h. After the reaction was completed, the reaction mixture was diluted with EtOAc and extracted with water. Extracted organic layer was dried over magnesium sulfate and concentrated under reduced pressure. The crude product was then purified by HPLC to give the titled compound as yellow solid (32 mg, 66%). ¹H NMR (400 MHz, DMSO-d₆) δ ppm 1.70 (s, 3H) 4.46 (d, J=5.27 Hz, 2H) 4.89 (d, J=5.27 Hz, 2H) 7.21-7.24 (m, 2H) 7.68-7.72 (m, 1H) 7.92 (br. s., 2H) 8.01 (d, J=6.59 Hz, 1H) 8.32 (m, 1H). m/z (ESI-HRMS) calculated for C₁₈H₁₈ON₅=320.1433 found=320.1505.

### Example 142

### 5-(2-Aminopyridin-4-yl)-7-((tetrahydro-2H-pyran-4-yl)ethynyl)-1H-indazol-3-amine

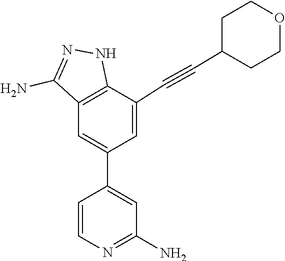 [SMILES: Nc1cc(-c2cc(C#CC3CCOCC3)c3[nH]nc(N)c3c2)ccn1]

In a sealed tube, a solution of 5-(2-aminopyridin-4-yl)-7-bromo-1H-indazol-3-amine (0.05 g, 0.15 mmol), copper (I) iodide (0.0014 g, 0.0075 mmol) and [1,1′-bis(diphenylphosphino)ferrocene]dichloropalladium(II) catalyst (0.005 g, 0.0075 mmol) in 0.45 ml of tetrahydrofuran:triethylamine (1:4) was degassed followed by addition of 4-ethynyltetrahydro-2H-pyran (0.02 ml, 0.22 mmol). The reaction mixture was stirred at room temperature for 16 h. After the reaction was completed, the reaction mixture was diluted with EtOAc and extracted with water. Extracted organic layer was dried over magnesium sulfate and concentrated under reduced pressure. The crude product was then purified by HPLC to give the titled compound as yellow solid (41.8 mg, 84%). ¹H NMR (400 MHz, DMSO-d₆) b ppm 1.67-1.78 (m, 2H) 1.86-1.94 (m, 2H) 2.98 (tt, J=8.90, 4.28 Hz, 1H) 3.48 (ddd, J=11.53, 9.12, 2.64 Hz, 2H) 3.86 (dt, J=11.75, 4.23 Hz, 2H) 7.19-7.25 (m, 2H) 7.67 (d, J=1.76 Hz, 1H) 7.93 (br. s., 2H) 8.00 (d, J=7.03 Hz, 1H) 8.30 (d, J=1.30 Hz, 1H) 12.12 (br. s., 1H). m/z (ESI-HRMS) calculated for C₉H₂₀ON₅=334.1590 found=334.1655.

### Example 143

### 5-(2-Aminopyrimidin-4-yl)-7-bromo-1H-indazol-3-amine

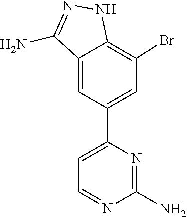 [SMILES: Nc1nccc(-c2cc(Br)c3[nH]nc(N)c3c2)n1]

To a solution of 4-iodopyrimridin-2-amine (0.3 g, 1.38 mmol) in a degassed solution of dioxane:H₂O (18:2 5 mL) was added cesium carbonate (1.35 g, 4.14 mmol), 7-bromo-5-(4,4,5,5-tetramethyl-1,3,2-dioxaborolan-2-yl)-1H-indazol-3-amine (0.558 g, 1.66 mmol) and bis(triphenylphosphine)palladium(II) dichloride catalyst (0.097 g, 0.138 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by HPLC to give the desired compound as a dark brown solid (0.07 g, 17%). ¹H NMR (500 MHz, DMSO-d₆) δ 12.04 (s, 1H), 8.52 (s, 1H), 8.35-8.26 (m, 2H), 7.09 (d, J=5.3 Hz, 1H), 6.62 (s, 2H), 5.69 (s, 2H). LRMS: Calculated for C₁₁H₉BrN₆ 304.01; Found: 307.0; 305.0 (M+H)

### 5-(2-Aminopyrimidin-4-yl)-7-(3,3-dimethylbut-1-yn-1-yl)-1H-indazol-3-amine

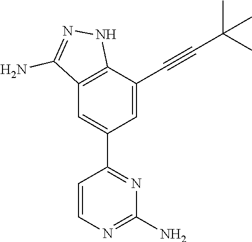 [SMILES: CC(C)(C)C#Cc1cc(-c2ccnc(N)n2)cc2c(N)n[nH]c12]

To a solution of 5-(2-aminopyrimidin-4-yl)-7-bromo-1H-indazol-3-amine (0.05 g, 0.16 mmol) in a degassed 4:1 solution of DMF:triethylamine (5 mL) was added [1,1′-bis(diphenylphosphino)ferrocene]dichloropalladium(II) catalyst (0.011 g, 0.016 mmol) and 3,3-dimethylbut-1-yne (0.017 g, 0.2 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether:EtOAc to afford the target compound as a brown solid (0.013 g, 26%). NMR ¹H NMR (500 MHz, DMSO-d₆) δ 11.87 (s, 1H), 8.27 (d, J=5.3 Hz, 1H), 7.07 (d, J=5.3 Hz, 1H), 6.58 (s, 2H), 5.62 (s, 2H), 1.37 (s, 9H).

LRMS: Calculated for C₁₇H₁₈N₆ 306.16; Found 307.16 (M+H)

### Example 144

### 5-(2-Aminopyridin-4-yl)-7-(3,3-dimethylbutyl)-1H-indazol-3-amine

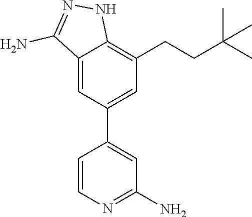 [SMILES: CC(C)(C)CCc1cc(-c2ccnc(N)c2)cc2c(N)n[nH]c12]

To a solution of 5-(2-aminopyridin-4-yl)-7-(3,3-dimethylbut-1-yn-1-yl)-1H-indazol-3-amine (Example 137) (30 mg, 0.098 mmol, 1 Eq) in dry MeOH (2 mL) was added 10% Pd/C (10.42 mg, 0.098 mmol, 1 Eq). The reaction mixture was allowed to stir under H₂ atmosphere at room temperature overnight.

The reaction was then diluted with MeOH, filtered through celite, concentrated under reduced pressure and the crude residue was purified by flash column chromatography eluting with a gradient of EtOAc:Petroleum Ether (0-80%) to afford the target compound (15 mg, 0.048 mmol, 50%). ¹H NMR (500 MHz, DMSO-d6) δ 11.56 (s, 1H), 7.92 (d, J=5.5 Hz, 1H), 7.87 (d, J=1.7 Hz, 1H), 7.30 (d, J=1.7 Hz, 1H), 6.79 (dd, J=5.5, 1.7 Hz, 1H), 6.72 (d, J=1.8 Hz, 1H), 5.88 (s, 2H), 5.42 (s, 2H), 2.81-2.73 (m, 2H), 1.62-1.55 (m, 2H), 0.99 (s, 9H) ppm.

LRMS: For C₁₈H₂₃N₅ requires 309.41 found 310.3 (M+H).

### Example 145

### 5-(2-Aminopyridin-4-yl)-7-(2-cyclohexylethyl)-1H-indazol-3-amine SU1672

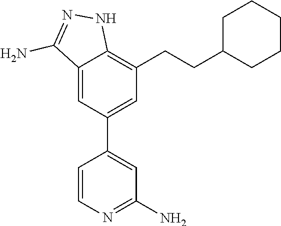 [SMILES: Nc1cc(-c2cc(CCC3CCCCC3)c3[nH]nc(N)c3c2)ccn1]

To a solution of 5-(2-aminopyridin-4-yl)-7-(cyclohexylethynyl)-1H-indazol-3-amine (35 mg, 0.11 mmol) in 1.0 ml of dry MeOH was added 10% Pd/C (17 mg, 0.16 mmol). The reaction was stirred at RT, o/n under a hydrogen atmosphere. The resulting mixture was diluted with EtOAc, filtered through silica, concentrated under reduced pressure and the residue purified by HPLC to afford the title compound as a white solid (32 mg, 0.09 mmol, 77%). 1H NMR (400 MHz, MeOD): δ 0.64-0.77 (m, 2H), 1.00-1.57 (m, 11H), 2.48-2.56 (m, 2H), 6.58 (d, J=5.2 Hz, 1H), 7.23 (dd, J=8.6, 1.6 Hz, 1H), 7.36 (dd, J=8.6, 0.8 Hz, 1H), 7.61 (app s, 1H), 7.78 (app s, 1H), 5 protons missing. LRMS: Calculated for C₂₀H₂₅N₅ 335.2 found 336.2 (M+1).

### Example 146

### 5-(2-Aminopyridin-4-yl)-7-(2-cyclopropylethyl)-1H-indazol-3-amine

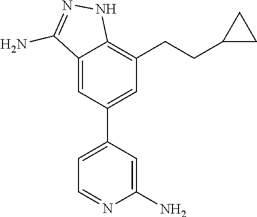 [SMILES: Nc1cc(-c2cc(CCC3CC3)c3[nH]nc(N)c3c2)ccn1]

To a solution of 5-(2-aminopyridin-4-yl)-7-(cyclopropylethynyl)-1H-indazol-3-amine (Example 136) (30 mg, 0.103 mmol, 1 Eq) in dry MeOH (2 mL) was added 10% Pd/C (10.96 mg, 0.103 mmol, 1 Eq). The reaction mixture was allowed to stir under H₂ atmosphere at room temperature overnight.

The reaction was then diluted with MeOH, filtered through celite, concentrated under reduced pressure and the crude residue was purified by flash column chromatography eluting with a gradient of EtOAc:Petroleum Ether (0-100%) followed by HPLC purification to afford the target compound (12.99 mg, 0.044 mmol, 43%). ¹H NMR (500 MHz, CD₃OD) δ 7.90 (d, J=5.6 Hz, 1H), 7.87 (br. s, 1H), 7.44 (d, J=1.7 Hz, 1H), 6.95 (dd, J=5.6, 1.7 Hz, 1H), 6.88 (d, J=1.7 Hz, 1H), 2.98 (t, J=7.6 Hz, 2H), 1.65 (q, J=7.0 Hz, 2H), 0.81-0.74 (m, 1H), 0.45-0.40 (m, 2H), 0.09-0.06 (m, 2H) ppm. LRMS (ESI+ve): For C₁₇H₁₉N₅ Molecular Weight: 293.37 found 294.1 (M+H).

### Example 147

### 5-(2-Aminopyridin-4-yl)-7-phenethyl-1H-indazol-3-amine

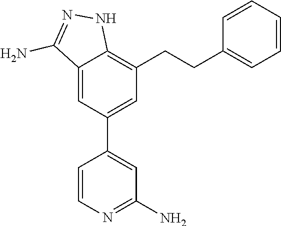 [SMILES: Nc1cc(-c2cc(CCc3ccccc3)c3[nH]nc(N)c3c2)ccn1]

Pd/C 5% (10 mg, 10%) was added to a solution of 5-(2-aminopyridin-4-yl)-7-(phenylethynyl)-1H-indazol-3-amine (100 mg, 0.31 mmol) in MeOH (5 mL). The reaction mixture was allowed to stir under H₂ atmosphere at rt for 18 h. Pd/C was filtered off through celite. The organic solvent was removed under reduced pressure. The crude residue was purified using column chromatography (5% MeOH in EtOAc) followed by HPLC purification (60% MeCN in H₂O) to give the titled product as yellow solid (70 mg, 0.21 mmol, 69%). ¹H NMR (500 MHz, DMSO-d6) δ 3.01 (dd, J=10.4, 6.0 Hz, 2H), 3.13 (dd, J=10.1, 6.2 Hz, 2H), 7.14-7.25 (m, 3H), 7.26-7.38 (m, 5H), 7.49 (d, J=1.7 Hz, 1H), 8.02 (d, J=6.8 Hz, 1H), 8.11 (s, 2H), 8.19 (d, J=1.7 Hz, 1H). LRMS: For C₂₀H₁₉N₅ requires 329.41 found 330.1 (M+H).

### Section 8—Compounds of the Formula

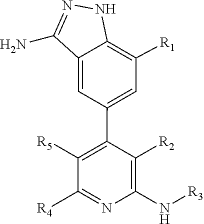

where R₂, R₄ and R₅ are H, F or NH₂.

### General Method for Preparation of 3-bromo-2-fluoro-5-(4,4,5,5-tetramethyl-1,3,2-dioxaborolan-2-yl)benzonitrile

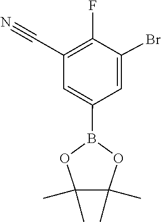 [SMILES: CC1(C)OB(c2cc(Br)c(F)c(C#N)c2)OC1(C)C]

Preparation of stock solution: [Ir(OMe)cod]₂ ([(1,5-cyclooctadiene) (methoxy) iridium (1) dimer]) (104 mg, 0.312 mmol), dtbpy (di-tert-butyl-2,2′dipyridyl) (84 mg, 0.312 mmol) and B₂pin₂ (bis (pinacolato) diboron) (2644 mg, 10.4 mmol) were mixed in volumetric flask and diluted up to 25 ml with MTBE (tert-butyl methylether). 2.5 ml of the stock solution (catalyst loading=3 mol %, 1.0 mmol B₂pin₂) was added to 3-bromo-2-fluorobenzonitrile (2.0 g, 10 mmol) in a microwave vial under nitrogen. The reaction was then degassed, sealed and heated to 80° C. overnight. The solvent was evaporated and residue purified by flash column chromatography eluting with a gradient of diethyl ether/petroleum ether (60-80%) 0-50% to give the target compound as a white solid 2.54 g, 78%. ¹H NMR (500 MHz, DMSO-d₆) δ 8.15 (dd, J=7.2, 1.5 Hz, 1H), 8.05 (dd, J=6.2, 1.5 Hz, 1H), 1.32 (s, 12H). ¹⁹F NMR (471 MHz, DMSO-d₆) δ −98.28. LRMS (ESI) m/z [M]⁺ 325.03, 327.03.

### General Method for Preparation of 7-bromo-5-(4,4,5,5-tetramethyl-1,3,2-dioxaborolan-2-yl)-1H-indazol-3-amine

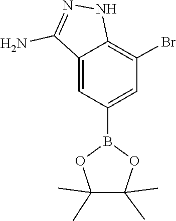 [SMILES: CC1(C)OB(c2cc(Br)c3[nH]nc(N)c3c2)OC1(C)C]

To a solution of 3-bromo-2-fluoro-5-(4,4,5,5-tetramethyl-1,3,2-dioxaborolan-2-yl)benzonitrile (2.0 g, 6.0 mmol) in ethanol (20 ml), hydrazine hydrate (22 ml, 340 mmol, 50-60%) was added and the reaction stirred at reflux for 18 h. The solvent was evaporated and the residue was triturated with 12 ml EtOAc and petroleum ether (1:1) and then filtered under vacuum and washed with water then petroleum ether 60-80% to give the desired compound as a yellow solid (1.51 g, 73%). ¹H NMR (500 MHz, DMSO-d₆) δ 11.93 (s, 1H), 8.19 (s, 1H), 7.60 (s, 1H), 5.68 (s, 2H), 1.31 (s, 12H). LRMS (ESI-MS) m/z [M]⁺ 337.01 (100%), 339.01 (100%).

### General Method for Preparation of 7-chloro-5-(4,4,5,5-tetramethyl-1,3,2-dioxaborolan-2-yl)-1H-indazol-3-amine

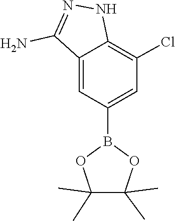 [SMILES: CC1(C)OB(c2cc(Cl)c3[nH]nc(N)c3c2)OC1(C)C]

To a solution of 3-chloro-2-fluoro-5-(4,4,5,5-tetramethyl-1,3,2-dioxaborolan-2-yl)benzonitrile (0.4 g, 1.4 mmol) in ethanol (20 ml), hydrazine hydrate (0.22 ml, 2.47 g, 6.8 mmol, 50-60%) was added and the reaction stirred at reflux for 18 h. The solvent was evaporated and the residue was triturated with 12 ml EtOAc and petroleum ether (1:1) and then filtered under vacuum and washed with water then petroleum ether 60-80% to give the desired compound as a yellow solid (320 mg, 75%). ¹H NMR (500 MHz, DMSO-d₆) δ 12.01 (s, 1H), 8.18-8.12 (m, 1H), 7.45 (s, 1H), 5.68 (s, 2H), 1.31 (s, 12H).

### Example 148

### 5-(2-Amino-5-fluoropyridin-4-yl)-7-(3,3-dimethylbut-1-yn-1-yl)-1H-indazol-3-amine

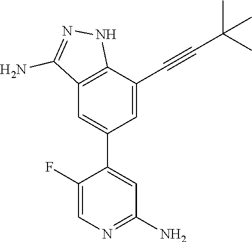 [SMILES: CC(C)(C)C#Cc1cc(-c2cc(N)ncc2F)cc2c(N)n[nH]c12]

A 0.5-2 mL MW tube was charged with a mixture of 5-(2-amino-5-fluoropyridin-4-yl)-7-chloro-1H-indazol-3-amine (33 mg, 0.12 mmol, 1 eq.), (3,3-dimethylbut-1-yn-1-yl)trifluoroborate (36 mg, 0.92 mmol, 1.6 eq.), XphosG2 (4.7 mg, 0.006 mmol, 5 mol %) in 1,4-dioxane (oxygen-free, 1.1 mL) was heated to 60° C. under a gentle flow of nitrogen for 5 minutes, then 2 M aq. K₂CO₃(oxygen-free, 0.36 mL, 0.72 mmol, 6 eq.) was added and the reaction mixture was heated to 110° C. for 12 hours. The reaction mixture was then cooled to room temperature, diluted 1 M aq. K₂CO₃ (50 mL), and extracted with EtOAc (50 mL). The organic layer was separated and washed 1 M aq. K₂CO₃ (50 mL), dried (MgSO₄) and purified by HPLC. ¹H NMR (400 MHz, DMSO) δ 1.35 (s, 9H), 6.87 (d, J=6.2 Hz, 1H), 7.42 (t, J=1.6 Hz, 1H), 8.05 (t, J=1.6 Hz, 1H), 8.12 (d, J=4.2 Hz, 1H), 12.00 (s, 1H). NH₂ peaks not observed. ¹⁹F NMR (471 MHz, DMSO) 5-74.10 (s, 3F),−148.76 (bs, 1F) HRMS: LRMS: Calculated for C₁₈H₁₈FN₅ 323.15; Found: 324.2 [M+H]⁺

### Example 149

### 5-(2-Amino-3-fluoropyridin-4-yl)-7-(3,3-dimethylbut-1-yn-1-yl)-1H-indazol-3-amine

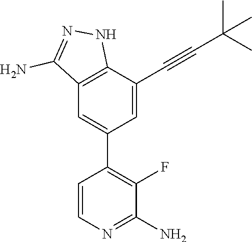 [SMILES: CC(C)(C)C#Cc1cc(-c2ccnc(N)c2F)cc2c(N)n[nH]c12]

A 0.5-2 mL MW tube was charged with a mixture of 5-(2-amino-3-fluoropyridin-4-yl)-7-chloro-1H-indazol-3-amine (33 mg, 0.12 mmol, 1 eq.), (3,3-dimethylbut-1-yn-1-yl)trifluoroborate (36 mg, 0.92 mmol, 1.6 eq.), XphosG2 (4.7 mg, 0.006 mmol, 5 mol %) in 1,4-dioxane (oxygen-free, 1.1 mL) was heated to 60° C. under a gentle flow of nitrogen for 5 minutes, then 2 M aq. K₂CO₃(oxygen-free, 0.36 mL, 0.72 mmol, 6 eq.) was added and the reaction mixture was heated to 110° C. for 12 hours. The reaction mixture was then cooled to room temperature, diluted 1 M aq. K₂CO₃ (50 mL), and extracted with EtOAc (50 mL). The organic layer was washed 1 M aq. K₂CO₃ (50 mL), dried (MgSO₄) and purified by flash chromatography (Silica, 50 g, 2-5% MeOH in AcOEt) to give the title compound as a pale yellow solid (16 mg, 17%). ¹H NMR (400 MHz, DMSO-D₆) δ 1.35 (s, 9H), 5.61 (s, 2H), 6.22 (s, 2H), 6.67 (t, J=5.1 Hz, 1H), 7.39 (d, J=1.7 Hz, 1H), 7.76 (d, J=5.0 Hz, 1H), 7.98 (s, 1H), 11.83 (s, 1H). LRMS: Calculated for C₁₈H₁₈FN₅ 323.15; Found: 324.3 [M+H]⁺

### Example 150

### 6-Fluoro-4-iodopyridin-2-amine

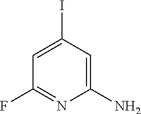

To a solution of 2, 6-difluoro-4-iodopyridine (1.15 g, 4.77 mmol) in dioxane (5 mL) was added ammonium hydroxide (10 mL 28% in H₂O). The reaction was the flushed with argon, sealed and stirred at 80° C. for 3 hours by microwave irradiation. The reaction was cooled, partitioned between EtOAc and H₂O and the aqueous layer washed a further 2 times with EtOAc (2×30 mL). The combined organic layers were washed with brine and concentrated under reduced pressure. Purification by flash column chromatography afforded the desired compound as a clear oil (0.90 g, 79%). ¹H NMR (400 MHz, DMSO-d₆) δ 7.61 (d, J=5.4 Hz, 1H), 6.87 (d, J=1.5 Hz, 1H), 6.82 (dd, J=5.3, 1.5 Hz, 1H), 6.08 (s, 2H).

### 5-(2-Amino-6-fluoropyridin-4-yl)-7-bromo-1H-indazol-3-amine

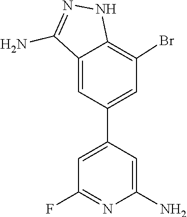 [SMILES: Nc1cc(-c2cc(Br)c3[nH]nc(N)c3c2)cc(F)n1]

To a solution of 6-fluoro-4-iodopyridin-2-amine (0.3 g, 1.26 mmol) in a degassed solution of dioxane:H₂O (18:2 5 mL) was added cesium carbonate (1.23 g, 3.78 mmol), 7-bromo-5-(4,4,5,5-tetramethyl-1,3,2-dioxaborolan-2-yl)-1H-indazol-3-amine (0.511 g, 1.51 mmol) and bis(triphenylphosphine)palladium(II) dichloride catalyst (0.44 g, 0.63 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a light brown solid (0.31 g, 76%). ¹H NMR (400 MHz, DMSO-d₆) δ 11.98 (s, 1H), 8.16 (d, J=1.5 Hz, 1H), 7.75 (d, J=1.5 Hz, 1H), 6.60 (t, J=1.6 Hz, 1H), 6.44 (d, J=1.1 Hz, 1H), 6.37 (s, 2H), 5.66 (s, 2H). LRMS (ESI+ve): For C₁₂H₉FBrN₅ Molecular weight 322.1414 found 323.0, 324.0.

### 5-(2-Amino-6-fluoropyridin-4-yl)-7-(3,3-dimethylbut-1-yn-1-yl)-1H-indazol-3-amine

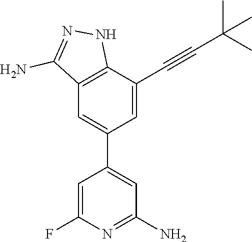 [SMILES: CC(C)(C)C#Cc1cc(-c2cc(N)nc(F)c2)cc2c(N)n[nH]c12]

To a solution of 5-(2-amino-6-fluoropyridin-4-yl)-7-bromo-1H-indazol-3-amine (0.31 g, 0.96 mmol) in a degassed 4:1 solution of DMF:triethylamine (5 mL) was added Copper iodide (0.18 g, 0.096 mmol), and [1,1′-bis(diphenylphosphino)ferrocene]dichloropalladium(II) as catalyst (0.067 g, 0.096 mmol) and 3,3-dimethylbut-1-yne (0.946 g, 0.142 mL, 1.15 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc followed by preparative HPLC to afford the target compound as a cream coloured powder (0.065 g. 21%). ¹H NMR (500 MHz, DMSO-d₆) δ 11.82 (s, 1H), 8.11 (d, J=1.6 Hz, 1H), 7.49 (d, J=1.6 Hz, 1H), 6.62 (d, J=1.5 Hz, 1H), 6.43 (d, J=1.1 Hz, 1H), 6.33 (s, 2H), 5.59 (s, 2H), 1.37 (s, 9H). ¹⁹F NMR (471 MHz, DMSO-d₆) δ −71.84. LRMS (ESI+ve): For C₁₈H₁₈FN₅ Molecular weight 323.3754 found 324.2.

### Example 151

### 4-Iodopyridine-2,6-diamine

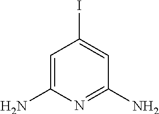

To a solution of 2, 6-difluoro-4-iodopyridine (0.5 g, 4.77 mmol) in dioxane (5 mL) was added Ammonium Hydroxide (10 mL 28% in H₂O) the reaction was the flushed with argon, sealed and stirred at 130° C. for 3 hours by microwave irradiation. The reaction was cooled, partitioned between EtOAc and H₂O and the aqueous layer washed a further 2 times with EtOAc (2×30 mL). The combined organic layers were washed with brine and concentrated under reduced pressure. Purification by flash column chromatography afforded the desired compound as a clear oil (0.4 g, 83%). ¹H NMR (500 MHz, DMSO-d₆) δ 6.01 (s, 2H), 5.56 (s, 4H). LRMS (ESI+ve): For C₅H₆IN₃ Molecular Weight: 234.9606 found 326.0

### 4-(3-Amino-7-bromo-1H-indazol-5-yl)pyridine-2,6-diamine

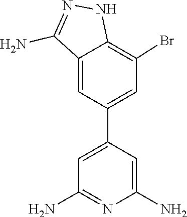 [SMILES: Nc1cc(-c2cc(Br)c3[nH]nc(N)c3c2)cc(N)n1]

To a solution of 4-iodopyridin-2,6-diamine (0.4 g, 1.7024 mmol) in a degassed solution of dioxane:H₂O (18:2 5 mL) was added cesium carbonate (1.664 g, 5.1072 mmol), 7-bromo-5-(4,4,5,5-tetramethyl-1,3,2-dioxaborolan-2-yl)-1H-indazol-3-amine (0.69 g, 2.043 mmol) and bis(triphenylphosphine)palladium(II) dichloride catalyst (0.060 g, 0.0851 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography NH silica cartridges from Biotage eluting from 50-100% petroleum ether—EtOAc+10% MeOH to afford the desired compound as a grey solid (0.487 g, 90%). ¹H NMR (500 MHz, DMSO-d₆) δ 7.78 (s, 1H), 7.38 (s, 1H), 5.73 (s, 2H), 5.42 (s, 2H), 5.24 (s, 4H).LRMS (ESI+ve): For C₁₂H₁₁BrN₆ Molecular Weight: 318.00229 found 319.0, 320.0

### 4-(3-Amino-7-(3,3-dimethylbut-1-yn-1-yl)-1H-indazol-5-yl)pyridine-2,6-diamine

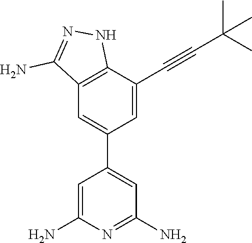 [SMILES: CC(C)(C)C#Cc1cc(-c2cc(N)nc(N)c2)cc2c(N)n[nH]c12]

To a solution of 4-(3-amino-7-bromo-1H-indazol-5-yl)pyridine-2,6-diamine (0.487 g, 1.531 mmol) in a degassed 4:1 solution of DMF:triethylamine (5 mL) was added [1,1′-bis(diphenylphosphino)ferrocene]dichloropalladium(II) catalyst (0.1075 g, 0.1531 mmol) and 3,3-dimethylbut-1-yne (0.1509 g, 0.100 mL, 1.837 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether—EtOAc+10% MeOH to afford the target compound as a greyish brown solid (0.274 g, 56%). ¹H NMR (500 MHz, DMSO-d₆) δ 11.70 (s, 1H), 7.93 (d, J=1.6 Hz, 1H), 7.37 (d, J=1.6 Hz, 1H), 5.95 (s, 2H), 5.54 (s, 2H), 5.41 (s, 4H), 1.36 (s, 9H). LRMS (ESI+ve): For C₁₈H₂₀N₆ Molecular weight 320.4000 found 321.2.

### Example 153

### N-Cyclopropyl-4-iodopyridin-2-amine

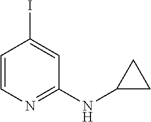 [SMILES: Ic1ccnc(NC2CC2)c1]

To a solution of 2-fluoro-4-iodopyridine (0.45 g, 2.02 mmol) in dioxane (5 mL) was added cyclopropylamine (0.28 mL, 4.05 mmol) the reaction was the flushed with argon, sealed and stirred at 130 for 3 hours. The reaction was cooled, partitioned between EtOAc and H₂O and the aqueous layer washed a further 2 times with EtOAc (2×30 mL). The combined organic layers were washed with brine and concentrated under reduced pressure. Purification by flash column chromatography afforded the desired compound as an off white solid (0.2 g, 38%). ¹H NMR (500 MHz, DMSO-d₆) δ 7.71 (d, J=5.3 Hz, 1H), 6.97 (d, J=1.4 Hz, 1H), 6.94 (d, J=2.5 Hz, 1H), 6.90 (dd, J=5.2, 1.5 Hz, 1H), 2.47 (dq, J=6.6, 3.4 Hz, 1H), 0.69 (td, J=6.7, 4.5 Hz, 2H), 0.43-0.38 (m, 2H). LRMS (ESI+ve): For C₈H₉IN₂ Molecular Weight: 260.0785 found 261.1

### 7-Bromo-5-(2-(cyclopropylamino)pyridin-4-yl)-1H-indazol-3-amine

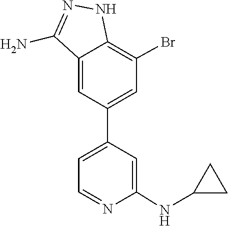 [SMILES: Nc1n[nH]c2c(Br)cc(-c3ccnc(NC4CC4)c3)cc12]

To a solution of N-cyclopropyl-4-iodopyridin-2-amine (0.2 g, 0.769 mmol) in a degassed solution of dioxane:H₂O (18:2 5 mL) was added cesium carbonate (0.751 g, 2.307 mmol), 7-bromo-5-(4,4,5,5-tetramethyl-1,3,2-dioxaborolan-2-yl)-1H-indazol-3-amine (0.312 g, 0.923 mmol) and bis(triphenylphosphine)palladium(II) dichloride catalyst (0.054 g, 0.0769 mmol) under an atmosphere of argon. The reaction was then sealed and stirred at 90° C. for 18 hours.

The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a yellow solid (0.032 g, 12%). ¹H NMR (500 MHz, DMSO-d₆) δ 11.93 (s, 1H), 8.14 (d, J=1.4 Hz, 1H), 8.04 (d, J=5.3 Hz, 1H), 7.76 (d, J=1.4 Hz, 1H), 6.86 (dd, J=5.2, 1.6 Hz, 1H), 6.83 (d, J=1.6 Hz, 1H), 6.77 (d, J=2.6 Hz, 1H), 5.66 (s, 2H), 2.60 (tq, J=6.6, 3.4 Hz, 1H), 0.75 (td, J=6.7, 4.6 Hz, 2H), 0.50-0.43 (m, 2H). LRMS (ESI+ve): For C₁₅H₁₄BrN₅ Molecular Weight: 344.2160 found 344.0, 345.0

### 5-(2-(Cyclopropylamino)pyridine-4-yl)-7-(3,3-dimethylbut-1-yn-1-yl)-1H-indazol-3-amine

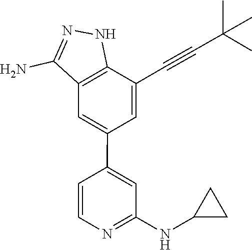 [SMILES: CC(C)(C)C#Cc1cc(-c2ccnc(NC3CC3)c2)cc2c(N)n[nH]c12]

To a solution of 7-bromo-5-(2-(cyclopropylamino)pyridin-4-yl)-1H-indazol-3-amine (0.32 g, 0.93 mmol) in a degassed 4:1 solution of DMF:triethylamine (5 mL) was added [1,1′-bis(diphenylphosphino)ferrocene]dichloropalladium(II) catalyst (0.068 g, 0.093 mmol), copper iodide (0.035 g, 0.186 mmol) and 3,3-dimethylbut-1-yne (0.0912 g, 0.135 mL, 1.110 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by HPLC to afford the target compound as a yellow (0.012 g. 4%). ¹H NMR (500 MHz, DMF-d₇) δ 13.59 (s, 1H), 12.48 (s, 1H), 9.31 (s, 1H), 8.71 (d, J=1.7 Hz, 1H), 8.45 (d, J=6.8 Hz, 1H), 8.04 (d, J=1.7 Hz, 1H), 7.71 (dd, J=6.8, 1.8 Hz, 1H), 7.65 (s, 1H), 1.79 (s, 9H), 1.38 (dt, J=7.0, 3.4 Hz, 2H), 1.09 (dt, J=7.2, 3.6 Hz, 2H). LRMS (ESI+ve): For C₂₁H₂₃N₅ Molecular Weight: 345.4500 found 346.2.

### Example 154

### N-Cyclobutyl-4-iodopyridin-2-amine

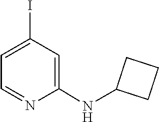 [SMILES: Ic1ccnc(NC2CCC2)c1]

In a sealed 10 mL microwave vial, 2-fluoro-4-iodopyridine (200 mg, 0.89 mmol, 1 Eq) was dissolved in 1 mL of Dioxane. Then cyclobutylamine (255.16 mg, 0.3 mL, 3.58 mmol, 4 Eq) was added and the resulting reaction mixture was heated at 150° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (20% EtOAc in petroleum ether) to give the titled product (170.7 mg, 0.62 mmol, 70%). ¹H NMR (500 MHz, DMSO-d₆) δ 7.65 (d, J=5.2 Hz, 1H), 6.90 (d, J=7.2 Hz, 1H), 6.81-6.79 (m, 2H), 4.21 (sxt, J=7.78 Hz, 1H), 2.24 (dtt, J=13.6, 5.1, 5.1, 2.8, 2.8 Hz, 2H), 1.87-1.79 (m, 2H), 1.70-1.60 (m, 2H) ppm.

### 7-Bromo-5-(2-(cyclobutylamino)pyridin-4-yl)-1H-indazol-3-amine

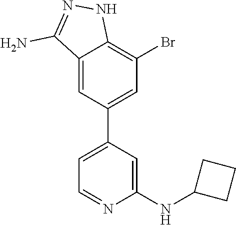 [SMILES: Nc1n[nH]c2c(Br)cc(-c3ccnc(NC4CCC4)c3)cc12]

To a solution of 7-bromo-5-(4,4,5,5-tetramethyl-1,3,2-dioxaborolan-2-yl)-1H-indazol-3-amine (230.5 mg, 0.68 mmol, 1.1 Eq) in a degassed 9:1 solution of dioxane/water (5 mL), were added N-cyclobutyl-4-iodopyridin-2-amine (170.7 mg, 0.62 mmol, 1 Eq), cesium carbonate (606.02 mg, 1.86 mmol, 3 Eq) and bis(triphenylphosphine)palladium chloride (43.52 mg, 0.062 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (100% EtOAc), to afford the titled compound as a solid (185 mg, 0.514 mmol, 83%). ¹H NMR (500 MHz, DMSO-d₆) δ 11.92 (br. s., 1H), 8.10 (d, J=1.2 Hz, 1H), 7.99 (d, J=5.3 Hz, 1H), 7.73-7.72 (m, 1H), 6.78 (dd, J=5.42, 1.45 Hz, 1H), 6.74 (d, J=7.48 Hz, 1H), 6.64 (s, 1H), 5.64 (br. s., 2H), 4.33 (sxt, J=7.84 Hz, 1H), 2.33-2.28 (m, 2H), 1.93-1.85 (m, 2H), 1.73-1.63 (m, 2H) ppm.

### 5-(2-(Cyclobutylamino)pyridin-4-yl)-7-(3,3-dimethylbut-1-yn-1-yl)-1H-indazol-3-amine

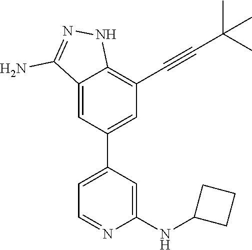 [SMILES: CC(C)(C)C#Cc1cc(-c2ccnc(NC3CCC3)c2)cc2c(N)n[nH]c12]

To a solution of 7-bromo-5-(2-(cyclobutylamino)pyridin-4-yl)-1H-indazol-3-amine (185 mg, 0.514 mmol, 1 Eq) in a degassed 4:1 solution of DMF/triethylamine (2 mL) were added copper(I) iodide (19.57 mg, 0.102 mmol, 0.2 Eq), [1,1′-bis(diphenylphosphino)ferrocene]dichloropalladium(II) catalyst (37.61 mg, 0.051 mmol, 0.1 Eq) and 3,3-dimethylbut-1-yne (63.33 mg, 0.949 mL, 0.771 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (20% to 90% EtOAc in petroleum ether) followed by HPLC purification to give the titled product (129.3 mg, 0.359 mmol, 70%). ¹H NMR (500 MHz, DMSO-d₆) δ 11.78 (br. s, 1H), 8.07 (d, J=1.6 Hz, 1H), 7.97 (d, J=5.5 Hz, 1H), 7.48 (d, J=1.6 Hz, 1H), 6.86 (br. s, 1H), 6.81 (dd, J=5.5, 1.6 Hz, 1H), 6.69 (s, 1H), 5.59 (br. s, 2H), 4.34 (h, J=7.8 Hz, 1H), 2.32 (qd, J=7.8, 5.3 Hz, 2H), 1.89 (pd, J=9.2, 2.7 Hz, 2H), 1.73-1.63 (m, 2H), 1.36 (s, 9H) ppm. LRMS (ESI+ve): For C₂₂H₂₅N₅ requires 359.48 found 360.3 (M+H).

### Example 155

### 4-Iodo-N-(oxetan-3-yl)pyridin-2-amine

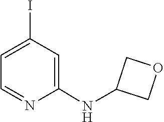 [SMILES: Ic1ccnc(NC2COC2)c1]

In a sealed 10 mL microwave vial, 2-fluoro-4-iodopyridine (200 mg, 0.89 mmol, 1 Eq) was dissolved in 3 mL of DMSO. Then 3-aminooxetane (97.57 mg, 0.936 mL, 1.78 mmol, 1.5 Eq) was added and the resulting reaction mixture was heated at 100° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (110.57 mg, 0.40 mmol, 45%). ¹H NMR (500 MHz, DMSO-d₆) δ 7.67 (d, J=5.3 Hz, 1H), 7.39 (d, J=6.0 Hz, 1H), 6.95-6.83 (m, 2H), 4.88-4.80 (m, 1H), 4.77 (dd, J=7.3, 5.8 Hz, 2H), 4.40 (t, J=6.1 Hz, 2H). ppm.

### 7-(3,3-Dimethylbut-1-yn-1-yl)-5-(4,4,5,5-tetramethyl-1,3,2-dioxaborolan-2-yl)-1H-indazol-3-amine

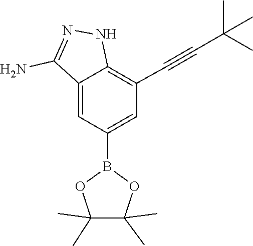 [SMILES: CC(C)(C)C#Cc1cc(B2OC(C)(C)C(C)(C)O2)cc2c(N)n[nH]c12]

A solution of 7-bromo-5-(4,4,5,5-tetramethyl-1,3,2-dioxaborolan-2-yl)-1H-indazol-3-amine (1.00 g, 3.0 mmol), copper (I) iodide (0.100 g, 0.5 mmol) and bis(triphenylphosphine) palladium(II) chloride (0.220 g, 0.3 mmol) in 10 ml of N,N-dimethyformamide-triethylamine (4:1) was degassed in sealed tube followed by addition of 3,3-dimethylbut-1-yne (1.46 mL, 17.8 mmol). The reaction mixture was heated to 80° C. for 1h20. The resulting mixture was diluted with EtOAc, filtered through silica, concentrated under reduced pressure and the residue was purified by column chromatography (first column: petroleum ether 60-80%/EtOAc 6:4; second column: DCM, 2% MeOH) to give the product as a dark green solid. (0.65 g, 64%). ¹H NMR (400 MHz, (CD₃)₂CO): δ 1.34 (s, 12H), 1.37 (s, 9H), 5.10 (br s, 2H), 7.64 (d, J=0.9 Hz, 1H), 8.08 (d, J=0.9 Hz, 1H), 11.03 (br s, 1H). LRMS: Calculated for C₁₉H₂₆BN₃O₂ 339.2 found 340.1 (M+1).

### 7-(3,3-Dimethylbut-1-yn-1-yl)-5-(2-(oxetan-3-ylamino)pyridin-4-yl)-1H-indazol-3-amine

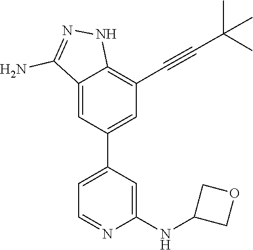 [SMILES: CC(C)(C)C#Cc1cc(-c2ccnc(NC3COC3)c2)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (149.27 mg, 0.44 mmol, 1.1 Eq) in a degassed 9:1 solution of dioxane/water (5 mL), were added 4-iodo-N-(oxetan-3-yl)pyridin-2-amine (110.57 mg, 0.40 mmol, 1 Eq), cesium carbonate (390.98 mg, 1.2 mmol, 3 Eq) and bis(triphenylphosphine)palladium chloride (28.07 mg, 0.04 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a pale yellow solid (73.73 mg, 0.204 mmol, 51%). ¹H NMR (500 MHz, DMSO-d₆) δ 11.79 (s, 1H), 8.07 (d, J=1.7 Hz, 1H), 7.99 (d, J=5.4 Hz, 1H), 7.48 (d, J=1.6 Hz, 1H), 7.23 (d, J=6.3 Hz, 1H), 6.85 (dd, J=5.4, 1.7 Hz, 1H), 6.75 (d, J=1.6 Hz, 1H), 5.58 (s, 2H), 4.96 (h, J=6.6 Hz, 1H), 4.83 (t, J=6.7 Hz, 2H), 4.46 (t, J=6.2 Hz, 2H), 1.36 (s, 9H) ppm. LRMS (ESI+ve): For C₂₁H₂₃N₅O requires 361.45 found 362.3 (M+H).

### Example 156

### N-Cyclopentyl-4-iodopyridin-2-amine

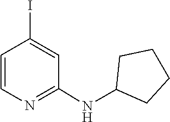 [SMILES: Ic1ccnc(NC2CCCC2)c1]

To a solution of 2-fluoro-4-iodopyridine (0.45 g, 2.02 mmol) in dioxane (5 mL) was added cyclopentylamine (0.189 g, 0.22 mL, 2.22 mmol) the reaction was the flushed with argon, sealed and stirred at 130° C. for 3 hours. The reaction was cooled, partitioned between EtOAc and H₂O and the aqueous layer washed a further 2 times with EtOAc (2×30 mL). The combined organic layers were washed with brine and concentrated under reduced pressure. Purification by flash column chromatography afforded the desired compound as an off white solid (0.178 g, 34%). LRMS (ESI+ve): For C₁₀H₁₃IN₂ Molecular Weight: 288.1325 found 289.1

### 7-Bromo-5-(2-(cyclopentylamino)pyridin-4-yl)-1H-indazol-3-amine

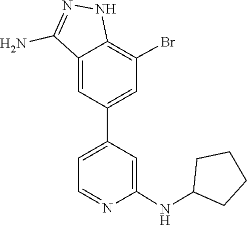 [SMILES: Nc1n[nH]c2c(Br)cc(-c3ccnc(NC4CCCC4)c3)cc12]

To a solution of N-cyclopentyl-4-iodopyridin-2-amine (0.18 g, 0.625 mmol) in a degassed solution of dioxane:H₂O (18:2 5 mL) was added potassium carbonate (0.270 g, 1.954 mmol), 7-bromo-5-(4,4,5,5-tetramethyl-1,3,2-dioxaborolan-2-yl)-1H-indazol-3-amine (0.253 g, 0.75 mmol) and bis(triphenylphosphine)palladium(II) dichloride catalyst (0.023 g, 0.031 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a bark brown solid (0.105 g, 45%).

### 5-(2-(Cyclopentylamino)pyridine-4-yl)-7-(3,3-dimethylbut-1-yn-1-yl)-1H-indazol-3-amine

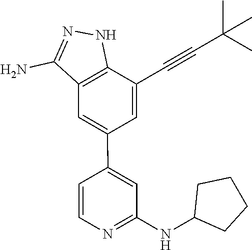 [SMILES: CC(C)(C)C#Cc1cc(-c2ccnc(NC3CCCC3)c2)cc2c(N)n[nH]c12]

To a solution of 7-bromo-5-(2-(cyclopentylamino)pyridin-4-yl)-1H-indazol-3-amine (0.10 g, 0.269 mmol) in a degassed 4:1 solution of DMF:triethylamine (5 mL) was added [1,1′-bis(diphenylphosphino)ferrocene]dichloropalladium(II) catalyst (0.010 g, 0.0134 mmol) and 3,3-dimethylbut-1-yne (0.0265 g, 0.040 mL, 0.323 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a yellow solid (0.018 g. 18%). ¹H NMR (500 MHz, DMSO-d₆) δ 8.71 (s, 1H), 8.31 (d, J=1.7 Hz, 1H), 7.97 (d, J=6.8 Hz, 1H), 7.65 (s, 1H), 7.28 (d, J=1.8 Hz, 1H), 7.21 (dd, J=6.9, 1.8 Hz, 1H), 2.05 (dd, J=12.5, 6.4 Hz, 2H), 1.74 (hept, J=4.9, 3.6 Hz, 2H), 1.67-1.53 (m, 4H), 1.37 (s, 9H). LRMS (ESI+ve): For Chemical Formula: C₂₃H₂₇N₅ Molecular Weight: 373.5040 found 374.3.

### Example 157

### N-(Cyclopropylmethyl)-4-iodopyridin-2-amine

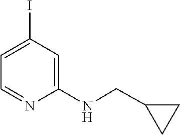 [SMILES: Ic1ccnc(NCC2CC2)c1]

In a sealed 10 mL microwave vial, 2-fluoro-4-iodopyridine (200 mg, 0.89 mmol, 1 Eq) was dissolved in 1 mL of Dioxane. Then cyclopropylmethylamine (126.59 mg, 0.154 mL, 1.78 mmol, 2 Eq) was added and the resulting reaction mixture was heated at 150° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (231.75 mg, 0.845 mmol, 95%). ¹H NMR (500 MHz, DMSO-d₆) δ 7.65 (d, J=5.3 Hz, 1H), 6.91 (d, J=1.2 Hz, 1H), 6.78 (dd, J=5.3, 1.4 Hz, 1H), 6.72 (t, J=5.3 Hz, 1H), 3.08 (dd, J=6.4, 5.8 Hz, 2H), 1.04-0.96 (m, 1H), 0.45-0.40 (m, 2H), 0.20-0.15 (m, 2H) ppm.

### 7-Bromo-5-(2-((cyclopropylmethyl)amino)pyridin-4-yl)-1H-indazol-3-amine

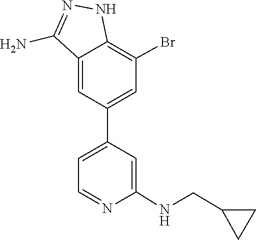 [SMILES: Nc1n[nH]c2c(Br)cc(-c3ccnc(NCC4CC4)c3)cc12]

To a solution of 7-bromo-5-(4,4,5,5-tetramethyl-1,3,2-dioxaborolan-2-yl)-1H-indazol-3-amine (311.99 mg, 0.923 mmol, 1.1 Eq) in a degassed 9:1 solution of dioxane/water (5 mL), were added N-(cyclopropylmethyl)-4-iodopyridin-2-amine (230 mg, 0.839 mmol, 1 Eq), cesium carbonate (820.08 mg, 2.517 mmol, 3 Eq) and bis(triphenylphosphine)palladium chloride (64.78 mg, 0.092 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (20% to 100% EtOAc in petroleum ether), to afford the titled compound as a solid (246.46 mg, 0.687 mmol, 82%). ¹H NMR (500 MHz, DMSO-d₆) 5 11.93 (br. s., 1H), 8.11 (d, J=1.2 Hz, 1H), 8.00 (d, J=5.3 Hz, 1H), 7.74-7.73 (m, 1H), 6.77 (dd, J=5.4, 1.3 Hz, 1H), 6.75 (s, 1H), 6.54 (t, J=5.6 Hz, 1H), 5.64 (br. s., 2H), 3.17 (t, J=6.2 Hz, 2H), 1.11-1.03 (m, 1H), 0.46-0.42 (m, 2H), 0.23-0.21 (m, 2H) ppm.

### 5-(2-((Cyclopropylmethyl)amino)pyridin-4-yl)-7-(3,3-dimethylbut-1-yn-1-yl)-1H-indazol-3-amine

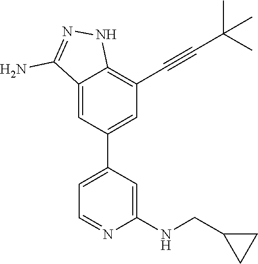 [SMILES: CC(C)(C)C#Cc1cc(-c2ccnc(NCC3CC3)c2)cc2c(N)n[nH]c12]

To a solution of 7-bromo-5-(2-((cyclopropylmethyl)amino)pyridin-4-yl)-1H-indazol-3-amine (240 mg, 0.669 mmol, 1 Eq) in a degassed 4:1 solution of DMF/triethylamine (2 mL) were added copper(I) iodide (25.51 mg, 0.133 mmol, 0.2 Eq), [1,1′-bis(diphenylphosphino)ferrocene]dichloropalladium(II) catalyst (48.95 mg, 0.0669 mmol, 0.1 Eq) and 3,3-dimethylbut-1-yne (82.42 mg, 0.123 mL, 1.003 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (20% to 100% EtOAc in petroleum ether) followed by HPLC purification to give the titled product (173.14 mg, 0.48 mmol, 72%). ¹H NMR (500 MHz, DMSO-d₆) δ 11.76 (br. s, 1H), 8.07 (d, J=1.7 Hz, 1H), 7.98 (d, J=5.4 Hz, 1H), 7.48 (d, J=1.6 Hz, 1H), 6.79 (d, J=1.6 Hz, 1H), 6.77 (s, 1H), 6.57 (s, 1H), 5.57 (s, 2H), 3.17 (dd, J=6.7, 5.6 Hz, 2H), 1.10-1.03 (m, 1H), 0.44-0.42 (m, 2H), 0.23-0.20 (m, 2H) ppm. LRMS (ESI+ve): For C₂₂H₂₅N₅ requires 359.48 found 360.3 (M+H).

### Example 158

### 4-Iodo-N-(2,2,2-trifluoroethyl)pyridin-2-amine

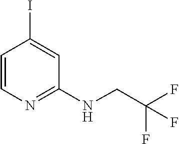 [SMILES: FC(F)(F)CNc1cc(I)ccn1]

Lithium bis(trimethylsilyl)amide (1M in hexane, 4.5 mL, 4.48 mmol, 2 Eq) was added dropwise to a solution of 2,2,2-trifluoroethan-1-amine (0.33 g, 0.3 mL, 3.36 mmol, 1.5 Eq) in anhydrous THF (5 mL) at −78° C. The reaction mixture was allowed to stir at −78° C. for 30 min. A solution of 2-fluoro-4-iodopyridine (0.5 g, 2.24 mmol, 1 Eq) in anhydrous THF (0.5 mL) was added dropwise at −78° C. The reaction mixture was stirred at rt for 3 h. The reaction was quenched with saturated solution of NH₄Cl (3 mL) then extracted between EtOAc (10 mL) and water (5 mL). The organic layer was washed with brine (2×5 mL), dried over anhydrous Na₂SO₄ and evaporated under reduced pressure. The crude residue was purified using column chromatography (10% EtOAc in petroleum ether) to give the titled product as white solid (0.2 g, 0.66 mmol, 30%). ¹H NMR (500 MHz, DMSO) δ 4.15 (qd, J=9.8, 6.6 Hz, 2H), 6.97 (dd, J=5.4, 1.5 Hz, 1H), 7.08 (d, J=1.6 Hz, 1H), 7.27 (t, J=6.6 Hz, 1H), 7.75 (d, J=5.3 Hz, 1H).

### Example 159

### 3-((4-Iodopyridin-2-yl)amino)propanenitrile

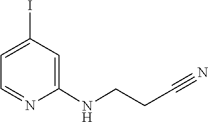 [SMILES: N#CCCNc1cc(I)ccn1]

In a sealed 10 mL microwave vial, 2-fluoro-4-iodopyridine (200 mg, 0.89 mmol, 1 Eq) was dissolved in 5 mL of DMSO. Then 3-Aminopropionitrile (stabilised with K₂CO₃) (93.57 mg, 0.982 mL, 1.335 mmol, 1.5 Eq) was added and the resulting reaction mixture was heated at 100° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (121.52 mg, 0.445 mmol, 50%). ¹H NMR (500 MHz, DMSO-d₆) δ 7.71 (d, J=5.3 Hz, 1H), 7.07 (t, J=5.9 Hz, 1H), 6.97 (d, J=1.4 Hz, 1H), 6.88 (dd, J=5.4, 1.5 Hz, 1H), 3.48 (q, J=6.3 Hz, 2H), 2.72 (t, J=6.5 Hz, 2H) ppm.

### 7-(3,3-Dimethylbut-1-yn-1-yl)-5-(4,4,5,5-tetramethyl-1,3,2-dioxaborolan-2-yl)-1H-indazol-3-amine

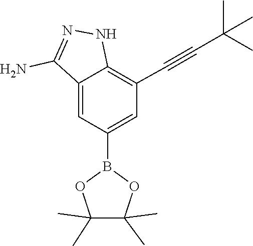 [SMILES: CC(C)(C)C#Cc1cc(B2OC(C)(C)C(C)(C)O2)cc2c(N)n[nH]c12]

A solution of 7-bromo-5-(4,4,5,5-tetramethyl-1,3,2-dioxaborolan-2-yl)-1H-indazol-3-amine (1.00 g, 3.0 mmol), copper (1) iodide (0.100 g, 0.5 mmol) and bis(triphenylphosphine) palladium(II) chloride (0.220 g, 0.3 mmol) in 10 ml of N,N-dimethyformamide-triethylamine (4:1) was degassed in sealed tube followed by addition of 3,3-dimethylbut-1-yne (1.46 mL, 17.8 mmol). The reaction mixture was heated to 80° C. for 1h20. The resulting mixture was diluted with EtOAc, filtered through silica, concentrated under reduced pressure and the residue was purified by column chromatography (first column: petroleum ether 60-80%/EtOAc 6:4; second column: DCM, 2% MeOH) to give the product as a dark green solid. (0.65 g, 64%). ¹H NMR (400 MHz, (CD₃)₂CO): δ 1.34 (s, 12H), 1.37 (s, 9H), 5.10 (br s, 2H), 7.64 (d, J=0.9 Hz, 1H), 8.08 (d, J=0.9 Hz, 1H), 11.03 (br s, 1H). LRMS: Calculated for C₁₉H₂₆BN₃O₂ 339.2 found 340.1 (M+1).

### 3-((4-(3-Amino-7-(3,3-dimethylbut-1-yn-1-yl)-1H-indazol-5-yl)pyridin-2-yl)amino)propanenitrile

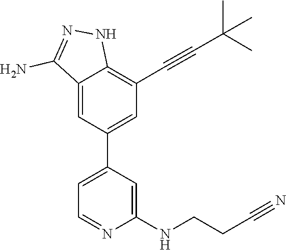 [SMILES: CC(C)(C)C#Cc1cc(-c2ccnc(NCCC#N)c2)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (166.06 mg, 0.489 mmol, 1.1 Eq) in a degassed 9:1 solution of dioxane/water (5 mL), were added 3-((4-iodopyridin-2-yl)amino)propanenitrile (121.52 mg, 0.445 mmol, 1 Eq), cesium carbonate (434.97 mg, 1.335 mmol, 3 Eq) and bis(triphenylphosphine)palladium chloride (31.23 mg, 0.044 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (127.60 mg, 0.356 mmol, 80%). ¹H NMR (500 MHz, DMSO-d₆) δ 11.77 (s, 1H), 8.08 (d, J=1.6 Hz, 1H), 8.04 (d, J=5.4 Hz, 1H), 7.49 (d, J=1.6 Hz, 1H), 6.90 (t, J=5.9 Hz, 1H), 6.86 (dd, J=5.5, 1.6 Hz, 1H), 6.80 (d, J=1.6 Hz, 1H), 5.58 (s, 2H), 3.56 (q, J=6.3 Hz, 2H), 2.78 (t, J=6.4 Hz, 2H), 1.36 (s, 9H) ppm. LRMS (ESI+ve): For C₂₁H₂₂N requires 358.45 found 359.3 (M+H).

### Example 160

### 2-((4-Iodopyridin-2-yl)amino)ethan-1-ol

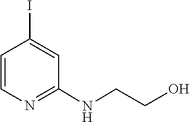 [SMILES: OCCNc1cc(I)ccn1]

A solution of 2-fluoro-4-iodopyridine (1 g, 4.49 mmol, 1 Eq), 2-aminoethan-1-ol (1.4 g, 1.4 mL, 22.45 mmol, 5 Eq) and triethylamine (1.4 g, 2 mL, 13.47 mmol, 3 Eq) in DMSO (5 mL) was stirred in a sealed vial (10 mL) at 95° C. for 18 h. Cold water (10 mL) was added to the reaction mixture. The produced precipitate was filtered and washed with cold water (2×5 mL) then petroleum ether (2×5 mL). The solid product was dried to give the titled product as white solid (0.6 g, 2.27 mmol, 51%). ¹H NMR (500 MHz, DMSO) δ 3.29 (q, J=5.9 Hz, 2H), 3.50 (q, J=5.8 Hz, 2H), 4.69 (t, J=5.4 Hz, 1H), 6.66 (t, J=5.8 Hz, 1H), 6.80 (dd, J=5.3, 1.5 Hz, 1H), 6.94 (d, J=1.4 Hz, 1H), 7.66 (d, J=5.3 Hz, 1H).

### 2-((4-(3-Amino-7-(3,3-dimethylbut-1-yn-1-yl)-1H-indazol-5-yl)pyridin-2-yl)amino)ethan-1-ol SU1697

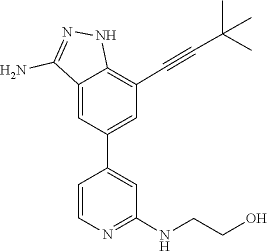 [SMILES: CC(C)(C)C#Cc1cc(-c2ccnc(NCCO)c2)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), 2-((4-iodopyridin-2-yl)amino)ethan-1-ol (0.4 g, 1.47 mmol, 1 Eq), cesium carbonate (1.4 g, 4.41 mmol, 3 Eq) and bis(triphenylphosphine)palladium chloride (157 mg, 0.22 mmol, 0.15 Eq) in dioxane (4 mL) and water (1 mL) was stirred under nitrogen atmosphere at 70° C. for 18 h. Cold water (5 mL) was added to the reaction mixture. The produced precipitate was filtered and washed with cold water (2×3 mL). The crude product was dried and purified using column chromatography (10% MeOH in EtOAc) followed by HPLC purification (60% MeCN in H₂O) to give the titled product as yellow solid (0.2 g, 0.57 mmol, 39%). ¹H NMR (500 MHz, DMSO) δ 1.37 (s, 9H), 3.38 (q, J=6.0 Hz, 2H), 3.56 (t, J=6.0 Hz, 2H), 4.76 (s, 1H), 5.57 (s, 2H), 6.45 (t, J=5.7 Hz, 1H), 6.75-6.82 (m, 2H), 7.48 (d, J=1.7 Hz, 1H), 7.99 (d, J=5.3 Hz, 1H), 8.07 (d, J=1.6 Hz, 1H), 11.76 (s, 1H). LRMS: For C₂₀H₂₃N₅O requires 349.44 found 350.3 (M+H).

### Example 161

### N¹-(4-Iodopyridin-2-yl)ethane-1,2-diamine

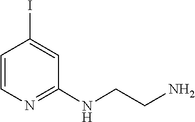 [SMILES: NCCNc1cc(I)ccn1]

A solution of 2-fluoro-4-iodopyridine (1 g, 4.49 mmol, 1 Eq), ethane-1,2-diamine (2.7 g, 3 mL, 45 mmol, 10 Eq) and triethylamine (1.4 g, 2 mL, 13.47 mmol, 3 Eq) in DMSO (5 mL) was stirred in a sealed vial (20 mL) at 95° C. for 18 h. Cold water (10 mL) was added to the reaction mixture. The produced precipitate was filtered and washed with cold water (2×5 mL) then petroleum ether (2×5 mL). The solid product was dried to give the titled product as yellow solid (0.4 g, 1.5 mmol, 33%). ¹H NMR (500 MHz, DMSO) δ 1.44 (s, 2H), 2.66 (t, J=6.4 Hz, 2H), 3.19 (q, J=6.2 Hz, 2H), 6.66 (t, J=5.6 Hz, 1H), 6.72-6.82 (m, 1H), 6.90 (d, J=1.4 Hz, 1H), 7.66 (d, J=5.3 Hz, 1H).

### N¹-(4-(3-Amino-7-(3,3-dimethylbut-1-yn-1-yl)-1H-indazol-5-yl)pyridin-2-yl)ethane-1,2-diamine SU1698

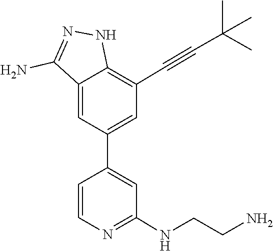 [SMILES: CC(C)(C)C#Cc1cc(-c2ccnc(NCCN)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), N¹-(4-iodopyridin-2-yl)ethane-1,2-diamine (0.39 g, 1.47 mmol, 1 Eq), cesium carbonate (1.4 g, 4.41 mmol, 3 Eq) and bis(triphenylphosphine)palladium chloride (157 mg, 0.22 mmol, 0.15 Eq) in dioxane (4 mL) and water (1 mL) was stirred under nitrogen atmosphere at 70° C. for 18 h. Cold water (5 mL) was added to the reaction mixture. The produced precipitate was filtered and washed with cold water (2×3 mL). The crude product was dried and purified using column chromatography (10% MeOH and 1% Et₃N in EtOAc) followed by HPLC purification (60% MeCN in H₂O) to give the titled product as yellow solid (0.1 g, 0.29 mmol, 20%). ¹H NMR (500 MHz, DMSO) δ 1.37 (s, 9H), 3.02 (d, J=6.4 Hz, 2H), 3.53 (d, J=6.1 Hz, 2H), 5.59 (s, 2H), 6.72 (d, J=5.7 Hz, 1H), 6.80 (s, 1H), 6.90 (d, J=5.5 Hz, 1H), 7.49 (d, J=1.6 Hz, 1H), 7.70 (s, 2H), 8.05 (d, J=5.4 Hz, 1H), 8.09 (d, J=1.6 Hz, 1H), 11.81 (s, 1H). LRMS: For C₂₀H₂₄N₆ requires 348.45 found 349.3 (M+H).

### Example 162

### 4-iodo-N-(2-methoxyethyl)pyridin-2-amine

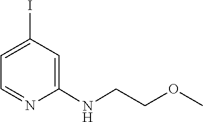

In a sealed 10 mL microwave vial, 2-fluoro-4-iodopyridine (200 mg, 0.89 mmol, 1 Eq) was dissolved in 1 mL of Dioxane. Then 2-methoxyethylamine (133.69 mg, 0.155 mL, 1.78 mmol, 2 Eq) was added and the resulting reaction mixture was heated at 150° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (240.1 mg, 0.86 mmol, 97%). ¹H NMR (500 MHz, DMSO-d₆) δ 7.66 (d, J=5.2 Hz, 1H), 6.94 (d, J=1.2 Hz, 1H), 6.79 (dd, J=5.3, 1.5 Hz, 1H), 6.71 (t, J=5.3 Hz, 1H), 3.43-3.41 (m, 2H), 3.39-3.36 (m, 2H), 3.25 (s, 3H) ppm.

### 7-bromo-5-(2-((2-methoxyethyl)amino)pyridin-4-yl)-1H-indazol-3-amine

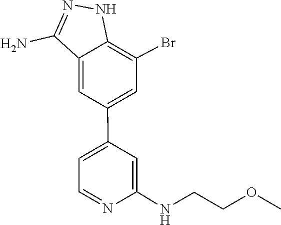 [SMILES: COCCNc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (307.51 mg, 0.909 mmol, 1.1 Eq) in a degassed 9:1 solution of dioxane/water (5 mL), were added 4-iodo-N-(2-methoxyethyl)pyridin-2-amine (230 mg, 0.827 mmol, 1 Eq), cesium carbonate (808.36 mg, 2.48 mmol, 3 Eq) and bis(triphenylphosphine)palladium chloride (58.05 mg, 0.082 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (100% EtOAc), to afford the titled compound as a solid (224.67 mg, 0.620 mmol, 75%). ¹H NMR (500 MHz, DMSO-d₆) δ 11.92 (br. s., 1H), 8.11 (d, J=1.4 Hz, 1H), 8.01 (d, J=5.3 Hz, 1H), 7.73 (d, J=1.4 Hz, 1H), 6.80-6.76 (m, 2H), 6.51-6.49 (m, 1H), 5.64 (br. s., 2H), 3.49-3.46 (m, 4H), 3.28 (s, 3H) ppm.

### 7-(3,3-dimethylbut-1-yn-1-yl)-5-(2-((2-methoxyethyl)amino)pyridin-4-yl)-1H-indazol-3-amine

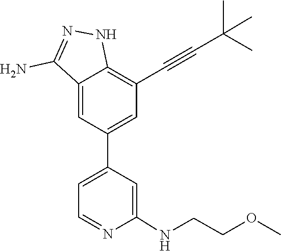 [SMILES: COCCNc1cc(-c2cc(C#CC(C)(C)C)c3[nH]nc(N)c3c2)ccn1]

To a solution of 7-bromo-5-(2-((2-methoxyethyl)amino)pyridin-4-yl)-1H-indazol-3-amine (220 mg, 0.607 mmol, 1 Eq) in a degassed 4:1 solution of DMF/triethylamine (2 mL) were added copper(I) iodide (23.12 mg, 0.12 mmol, 0.2 Eq), [1,1′-bis(diphenylphosphino)ferrocene]dichloropalladium(II) catalyst (44.41 mg, 0.0607 mmol, 0.1 Eq) and 3,3-dimethylbut-1-yne (74.78 mg, 0.112 mL, 0.91 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (20% to 100% EtOAc in petroleum ether) followed by HPLC purification to give the titled product (196.4 mg, 0.546 mmol, 90%). ¹H NMR (500 MHz, DMSO-d₆) δ 11.78 (br. s, 1H), 8.07 (d, J=1.6 Hz, 1H), 7.97 (d, J=5.5 Hz, 1H), 7.48 (d, J=1.6 Hz, 1H), 6.86 (br. s, 1H), 6.81 (dd, J=5.5, 1.6 Hz, 1H), 6.69 (s, 1H), 5.59 (br. s, 2H), 4.34 (h, J=7.8 Hz, 1H), 2.32 (qd, J=7.8, 5.3 Hz, 2H), 1.89 (pd, J=9.2, 2.7 Hz, 2H), 1.73-1.63 (m, 2H), 1.36 (s, 9H) ppm. LRMS (ESI+ve): For C₂₂H₂₅N₅ requires 359.48 found 360.3 (M+H).

### Example 163

### 4-Iodo-N-(3-methoxypropyl)pyridin-2-amine

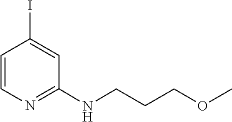

To anhydrous THF cooled to −78° C. was added nBuLi 4.5 mL (1 eq, 1M solution in THF) followed by DIA 0.633 mL (1eq) dropwise and then a solution of 2-fluoro-3-iodopyridine (1.0 g, 4.48 mmol) slowly. The reaction was allowed to warm up to room temperature and aged for 30 minutes at this temperature before cooling back down to −78° C. and quenching by pouring over ice: H₂O (20 mL). The reaction was then extracted with diethyl ether (3×50 mL), and the combined organic layers washed with brine dried over magnesium chloride and concentrated under reduced pressure to afford crude 2-fluoro-4-iodopyridine. This was then dissolved in dioxane (5 mL) in a 20 mL microwave vail to which 3-methoxypropan-1-amine was added seal and stirred at 120° C. under microwave irradiation for 3 hours. The reaction was cooled, partitioned between EtOAc and H₂O and the aqueous layer washed a further two times with EtOAc (2×30 mL). The combined organic layers were washed with brine and concentrated under reduced pressure. Purification by flash column chromatography afforded the desired compound as a white solid (0.43, 38%). ¹H NMR (500 MHz, DMSO-d₆) δ 7.84 (d, J=5.5 Hz, 1H), 6.76 (t, J=5.6 Hz, 1H), 6.67 (d, J=1.7 Hz, 1H), 6.63 (dd, J=5.5, 1.7 Hz, 1H), 3.37 (t, J=6.3 Hz, 2H), 3.26 (td, J=6.9, 5.6 Hz, 2H), 3.23 (s, 3H), 1.73 (p, J=6.6 Hz, 2H). LRMS (ESI+ve): For C₉H₁₃|N₂O Molecular Weight: 292.1205 found 293.0

### 7-Bromo-5-(2-((3-methoxypropyl)amino)pyridin-4-yl)-1H-indazol-3-amine

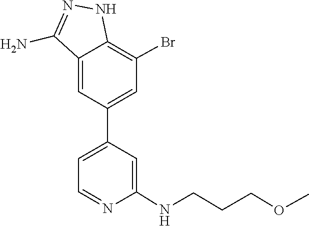 [SMILES: COCCCNc1cc(-c2cc(Br)c3[nH]nc(N)c3c2)ccn1]

To a solution of 4-iodo-N-(3-methoxypropyl)pyridin-2-amine (0.21 g, 0.73 mmol) in a degassed solution of dioxane:H₂O (18:2 5 mL) was added cesium carbonate (0.713 g, 2.188 mmol), 7-bromo-5-(4,4,5,5-tetramethyl-1,3,2-dioxaborolan-2-yl)-1H-indazol-3-amine (0.270 g, 0.802 mmol) and bis(triphenylphosphine)palladium(II) dichloride catalyst (0.026 g, 0.037 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.175 g, 64%). ¹H NMR (500 MHz, DMSO-d₆) δ 11.93 (s, 1H), 8.12 (d, J=1.4 Hz, 1H), 8.01 (d, J=5.4 Hz, 1H), 7.74 (d, J=1.4 Hz, 1H), 6.78 (dd, J=5.4, 1.6 Hz, 1H), 6.71 (d, J=1.6 Hz, 1H), 6.49 (t, J=5.6 Hz, 1H), 5.65 (s, 2H), 3.42 (t, J=6.3 Hz, 2H), 3.33 (d, J=6.2 Hz, 7H), 3.25 (s, 3H), 1.79 (p, J=6.6 Hz, 2H). LRMS (ESI+ve): For Chemical Formula: C₁₆H₁₈BrN₅O Molecular Weight: 376.2580 found 376.0, 377.0

### 7-(3,3-Dimethylbut-1-yn-1-yl)-5-(2-((3-methoxypropyl)amino)pyridine-4-yl)-1H-indazol-3-amine

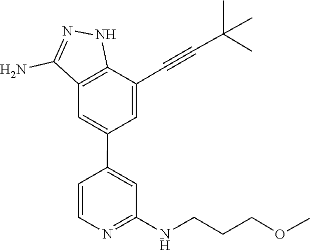 [SMILES: COCCCNc1cc(-c2cc(C#CC(C)(C)C)c3[nH]nc(N)c3c2)ccn1]

To a solution of 7-bromo-5-(2-((3-methoxypropyl)amino)pyridin-4-yl)-1H-indazol-3-amine (0.175 g, 0.466 mmol) in a degassed 4:1 solution of DMF:triethylamine (5 mL) was added [1,1′-bis(diphenylphosphino)ferrocene]dichloropalladium(II) catalyst (0.034 g, 0.046 mmol) and 3,3-dimethylbut-1-yne (0.046 g, 0.085 mL, 0.559 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a yellow solid (0.044 g. 25%). ¹H NMR (500 MHz, DMSO-d₆) δ 8.26 (t, J=1.6 Hz, 1H), 8.06 (d, J=6.5 Hz, 1H), 8.04 (d, J=1.7 Hz, 1H), 8.00 (t, J=1.5 Hz, 1H), 7.27 (s, 1H), 7.23 (dd, J=6.5, 1.7 Hz, 1H), 3.44 (t, J=6.1 Hz, 4H), 3.26 (s, 3H), 1.85 (p, J=6.5 Hz, 2H), 1.33 (s, 9H). LRMS (ESI+ve): For Chemical Formula: C₂₂H₂₇N₅O Molecular Weight: 377.4920 found 378.3

### Example 164

### N-(4-Iodopyridin-2-yl)acetamide

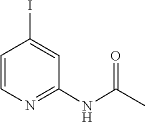

To a solution of 4-iodopyridin-2-amine (0.5 g 2.27 mmol) in dioxane cooled to 0° C. was added NaH (0.181 g, 4.54 mmol, 60% suspension, 2 eq) under a blanket of argon. Acetyl Chloride (0.18 g, 0.16 mmol) was then added dropwise and the resulting reaction mixture allowed to warm up to room temperature and stirred at this temperature for 3 hours. The reaction was then quenched by pouring over ice/water 20 mL, extracted with EtOAc (3×30 mL) and washed with brine (10 mL). The combined organic layers were concentrated and purified by flash eluting with a gradient of EtOAc:Petroleum Ether (50-100%) to afford the target compound as an off white solid (0.40 g, 67%). ¹H NMR (400 MHz, DMSO-d₆) δ 10.59 (s, 1H), 8.52 (d, J=1.7 Hz, 1H), 8.03 (dd, J=5.2, 0.6 Hz, 1H), 7.50 (dd, J=5.2, 1.5 Hz, 1H), 2.09 (s, 3H). LRMS (ESI+ve): For C₇H₇IN₂O Molecular Weight: 262.0505 found 263.4

### N-(4-(3-Amino-7-bromo-1H-indazol-5-yl)pyridin-2-yl)acetamide

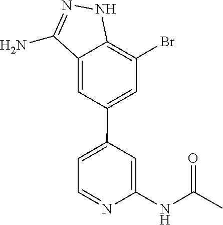 [SMILES: CC(=O)Nc1cc(-c2cc(Br)c3[nH]nc(N)c3c2)ccn1]

To a solution of N-(4-iodopyridin-2-yl)acetamide (0.4 g, 1.526 mmol) in a degassed solution of dioxane:H₂O (18:2 5 mL) was added cesium carbonate (1.492 g, 4.579 mmol), 7-bromo-5-(4,4,5,5-tetramethyl-1,3,2-dioxaborolan-2-yl)-1H-indazol-3-amine (0.617 g, 1.831 mmol) and bis(triphenylphosphine)palladium(II) dichloride catalyst (0.054 g, 0.0763 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.51 g, 96%). ¹H NMR (500 MHz, DMSO-d₆) δ 12.01 (s, 1H), 10.54 (s, 1H), 8.40 (s, 1H), 8.35 (d, J=5.3 Hz, 1H), 8.20 (d, J=1.4 Hz, 1H), 7.79 (s, 1H), 7.40 (dd, J=5.4, 1.7 Hz, 1H), 5.72 (s, 2H), 2.14 (s, 3H). LRMS (ESI+ve): C₁₄H₁₂BrN₅O Molecular Weight: 346.1880 found 346.0, 347.0

### N-(4-(3-Amino-7-(3,3-dimethylbut-1-yn-1-yl)-1H-indazol-5-yl)pyridin-2-yl)acetamide

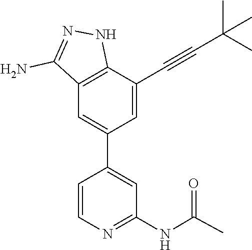 [SMILES: CC(=O)Nc1cc(-c2cc(C#CC(C)(C)C)c3[nH]nc(N)c3c2)ccn1]

To a solution of N-(4-(3-amino-7-bromo-1H-indazol-5-yl)pyridin-2-yl)acetamide (0.5 g, 1.444 mmol) in a degassed 4:1 solution of DMF:triethylamine (5 mL) was added [1,1′-bis(diphenylphosphino)ferrocene]dichloropalladium(II) catalyst (0.051 g, 0.072 mmol) and 3,3-dimethylbut-1-yne (0.14 g, 0.213 mL, 1.733 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc (plus NH₃ in MeOH 7M) (50-100%) to afford the target compound as a cream coloured solid (0.38 g. 76%). ¹H NMR (500 MHz, DMSO-d₆) δ 11.85 (s, 1H), 10.52 (s, 1H), 8.39 (s, 1H), 8.33 (d, J=5.3 Hz, 1H), 8.15 (d, J=1.6 Hz, 1H), 7.53 (d, J=1.6 Hz, 1H), 7.40 (dd, J=5.2, 1.7 Hz, 1H), 5.65 (s, 2H), 2.14 (s, 3H), 1.38 (s, 9H). LRMS (ESI+ve): C₂₀H₂₁N₅O Molecular Weight: 347.4220 found 348.3.

### Example 165

### N-(4-Iodopyridin-2-yl)propionamide

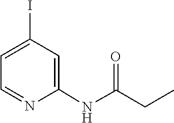

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise propionyl chloride (252.02 mg, 0.238 mL, 2.72 mmol, 1.2 Eq) at 5° C. The reaction mixture was allowed to stir at room temperature overnight. The reaction was then diluted with EtOAc (10 mL) and the organic layer was washed with water (2×5 mL), dried over anhydrous Na2SO4 and evaporated under reduced pressure. The crude residue was purified by column chromatography (60% DCM in EtOAc) to give the titled product as a solid (526.4 mg, 1.91 mmol, 84%). 1H NMR (500 MHz, DMSO-d₆) δ 10.53 (s, 1H), 8.55 (d, J=1.1 Hz, 1H), 8.02 (d, J=5.2 Hz, 1H), 7.48 (dd, J=5.2, 1.5 Hz, 1H), 2.39 (q, J=7.5 Hz, 2H), 1.05 (t, J=7.5 Hz, 3H) ppm.

### N-(4-(3-Amino-7-bromo-1H-indazol-5-yl)pyridin-2-yl)propionamide

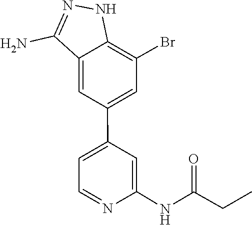 [SMILES: CCC(=O)Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (472.2 mg, 1.39 mmol, 1.1 Eq) in a degassed 9:1 solution of dioxane/water (5 mL), were added N-(4-iodopyridin-2-yl)propionamide (350 mg, 1.27 mmol, 1 Eq), cesium carbonate (1.241 g, 3.81 mmol, 3 Eq) and bis(triphenylphosphine)palladium chloride (89.14 mg, 0.127 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (100% EtOAc), to afford the titled compound as a solid (366 mg, 1.02 mmol, 80%). ¹H NMR (500 MHz, DMSO-d₆) δ 11.99 (br. s, 1H), 10.47 (s, 1H), 8.43 (s, 1H), 8.33 (d, J=5.2 Hz, 1H), 8.21 (d, J=1.5 Hz, 1H), 7.79 (d, J=1.5 Hz, 1H), 7.39 (dd, J=5.3, 1.8 Hz, 1H), 5.72 (s, 2H), 2.43 (q, J=7.6 Hz, 2H), 1.09 (t, J=7.6 Hz, 3H) ppm. LRMS (ESI+ve): For C₁₅H₁₄BrN₅O Molecular Weight: 360.22 found 360.0, 361.0, 362.0, 363.0 (M+H).

### N-(4-(3-Amino-7-(3,3-dimethylbut-1-yn-1-yl)-1H-indazol-5-yl)pyridin-2-yl)propionamide

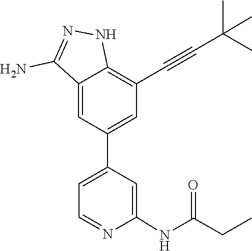 [SMILES: CCC(=O)Nc1cc(-c2cc(C#CC(C)(C)C)c3[nH]nc(N)c3c2)ccn1]

To a solution of N-(4-(3-amino-7-bromo-1H-indazol-5-yl)pyridin-2-yl)propionamide (250 mg, 0.694 mmol, 1 Eq) in a degassed 4:1 solution of DMF/triethylamine (2 mL) were added copper(I) iodide (26.43 mg, 0.138 mmol, 0.2 Eq), [1,1′-bis(diphenylphosphino)ferrocene]dichloropalladium(II) catalyst (50.78 mg, 0.069 mmol, 0.1 Eq) and 3,3-dimethylbut-1-yne (85.50 mg, 0.128 mL, 1.041 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (50% EtOAc in petroleum ether, then 100% EtOAc) followed by HPLC purification to give the titled product as a pale-yellow solid (178.2 mg, 0.493 mmol, 71%). ¹H NMR (500 MHz, DMSO-d₆) δ 11.83 (br. s, 1H), 10.46 (s, 1H), 8.42 (s, 1H), 8.31 (d, J=5.5 Hz, 1H), 8.15 (d, J=1.7 Hz, 1H), 7.52 (d, J=1.7 Hz, 1H), 7.38 (dd, J=5.3, 1.8 Hz, 1H), 5.65 (s, 2H), 2.43 (q, J=7.5 Hz, 2H), 1.37 (s, 9H), 1.09 (t, J=7.5 Hz, 3H) ppm. LRMS (ESI+ve): For C₂₁H₂₃N₅O Molecular Weight: 361.45 found 362.1 (M+H).

### Example 166

### tert-Butyl(7-bromo-5-(4,4,5,5-tetramethyl-1,3,2-dioxaborolan-2-yl)-1H-indazol-3-yl)(tert-butoxycarbonyl)carbamate

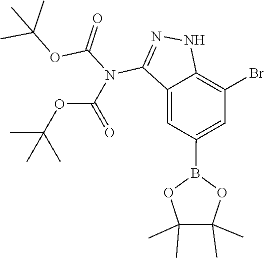 [SMILES: CC(C)(C)OC(=O)N(C(=O)OC(C)(C)C)c1n[nH]c2c(Br)cc(B3OC(C)(C)C(C)(C)O3)cc12]

Di-tert-butyl decarbonate (2.58 g, 2.7 mL, 11.84 mmol, 4 Eq) was added to a solution of 7-bromo-5-(4,4,5,5-tetramethyl-1,3,2-dioxaborolan-2-yl)-1H-indazol-3-amine (1 g, 2.96 mmol, 1 Eq), 4-(dimethylamino)pyridine (0.7 g, 5.92 mmol, 2 Eq), triethylamine (0.9 g, 1.2 mL, 8.88 mmol, 3 Eq) in anhydrous dioxane (5 mL). The reaction mixture was stirred at rt for 30 h. The reaction mixture was extracted between EtOAc (20 mL) and water (10 mL). The organic layer was washed with brine (2×5 mL), dried over anhydrous Na₂SO₄ and evaporated under reduced pressure. The crude residue was purified using column chromatography (10% EtOAc in petroleum ether) to give the titled product as white solid (0.6 g, 1.12 mmol, 38%). ¹H NMR (500 MHz, DMSO) δ 1.31 (s, 12H), 1.38 (s, 18H), 5.68 (s, 2H), 7.61 (s, 1H), 8.17 (s, 1H), 12.01 (s, 1H).

### tert-Butyl (5-(2-aminopyridin-4-yl)-7-bromo-1H-indazol-3-yl)(tert-butoxycarbonyl)carbamate

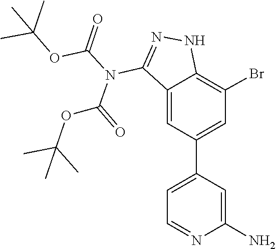 [SMILES: CC(C)(C)OC(=O)N(C(=O)OC(C)(C)C)c1n[nH]c2c(Br)cc(-c3ccnc(N)c3)cc12]

A solution of tert-butyl(7-bromo-5-(4,4,5,5-tetramethyl-1,3,2-dioxaborolan-2-yl)-1H-indazol-3-yl)(tert-butoxycarbonyl)carbamate (1 g, 1.86 mmol, 1 Eq), 4-iodopyridin-2-amine (0.41 g, 1.86 mmol, 1 Eq), cesium carbonate (1.82 g, 5.58 mmol, 3 Eq) and bis(triphenylphosphine)palladium chloride (197 mg, 0.28 mmol, 0.15 Eq) in dioxane (8 mL) and water (2 mL) was stirred under nitrogen atmosphere at 60° C. for 18 h. The reaction mixture was cooled and extracted between EtOAc (15 mL) and water (5 mL). The organic layer was washed with brine (2×5 mL), dried over anhydrous Na₂SO₄ and evaporated under reduced pressure. The crude residue was dried and purified using column chromatography (50% EtOAc in petroleum ether) to give the titled product as yellow solid (0.5 g, 0.99 mmol, 53%).

### tert-Butyl(5-(2-aminopyridin-4-yl)-7-(3,3-dimethylbut-1-yn-1-yl)-1H-indazol-3-yl)(tert-butoxycarbonyl)carbamate

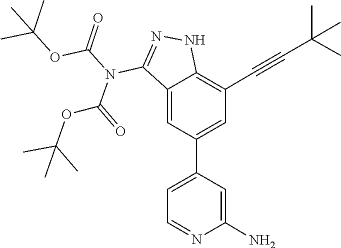 [SMILES: CC(C)(C)C#Cc1cc(-c2ccnc(N)c2)cc2c(N(C(=O)OC(C)(C)C)C(=O)OC(C)(C)C)n[nH]c12]

3,3-Dimethylbut-1-yne (163 mg, 240 μL, 1.98 mmol, 2 Eq) was added to a solution of tert-butyl (5-(2-aminopyridin-4-yl)-7-bromo-1H-indazol-3-yl)(tert-butoxycarbonyl)carbamate (0.5 g, 0.99 mmol, 1 Eq), copper(I) iodide (38 mg, 0.2 mmol, 0.2 Eq) and [1,1′-bis(diphenylphosphino)ferrocene]dichloropalladium(II) (73 mg, 0.1 mmol, 0.1 Eq) in anhydrous DMF (1 mL) and triethylamine (1 mL) under nitrogen atmosphere. The reaction mixture was allowed to stir at 50° C. for 18 h. The reaction mixture was extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) to give the titled product as yellow solid (0.3 g, 0.6 mmol, 60%).

### tert-Butyl(tert-butoxycarbonyl)(7-(3,3-dimethylbut-1-yn-1-yl)-5-(2-(3,3,3-trifluoropropanamido)pyridin-4-yl)-1H-indazol-3-yl)carbamate

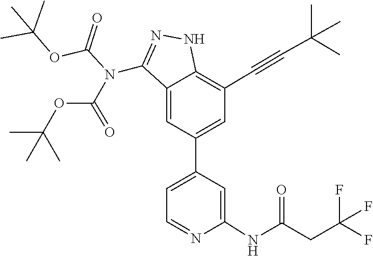 [SMILES: CC(C)(C)C#Cc1cc(-c2ccnc(NC(=O)CC(F)(F)F)c2)cc2c(N(C(=O)OC(C)(C)C)C(=O)OC(C)(C)C)n[nH]c12]

A solution of tert-butyl(5-(2-aminopyridin-4-yl)-7-(3,3-dimethylbut-1-yn-1-yl)-1H-indazol-3-yl)(tert-butoxycarbonyl)carbamate (0.5 g, 0.6 mmol, 1 Eq), (1-[bis(dimethylamino)methylene]-1H-1,2,3-triazolo[4,5-b]pyridinium 3-oxide hexafluorophosphate (274 mg, 0.72 mmol, 1.2 Eq), N,N-diisopropylethylamine (155 mg, 0.2 mL, 1.2 mmol, 2 Eq) in DMF (5 mL) was allowed to stir at rt for 18 h. The reaction mixture was extracted between EtOAc (10 mL) and water (5 mL).

The organic layer was washed with brine (2×5 mL), dried over anhydrous Na₂SO₄ and evaporated under reduced pressure. The crude residue was purified using column chromatography (40% EtOAc in petroleum ether) to give the titled product as purple crystals (0.3 g, 0.49 mmol, 82%).

### N-(4-(3-Amino-7-(3,3-dimethylbut-1-yn-1-yl)-1H-indazol-5-yl)pyridin-2-yl)-3,3,3-trifluoropropanamide

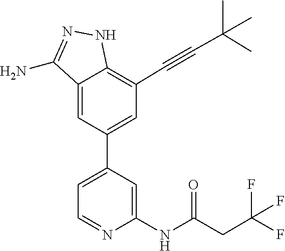 [SMILES: CC(C)(C)C#Cc1cc(-c2ccnc(NC(=O)CC(F)(F)F)c2)cc2c(N)n[nH]c12]

Trifluoroacetic acid (1.49 g, 1 mL, 13.1 mmol, 82 Eq) was added to a solution of tert-butyl(tert-butoxycarbonyl)(7-(3,3-dimethylbut-1-yn-1-yl)-5-(2-(3,3,3-trifluoropropanamido)pyridin-4-yl)-1H-indazol-3-yl)carbamate (100 mg, 0.16 mmol, 1 Eq) in DCM (4 mL) at 0° C. The reaction mixture was stirred at rt for 3 h. The reaction was quenched with saturated solution of NaHCO₃ (5 mL).

The organic layer was washed with brine (2×3 mL), dried over anhydrous Na₂SO₄ and evaporated under reduced pressure. The crude residue was purified using column chromatography (60% EtOAc in petroleum ether) followed by HPLC purification (70% MeCN in H₂O) to give the titled product as yellow solid (20 mg, 0.05 mmol, 31%). ¹H NMR (500 MHz, DMSO) δ 1.38 (s, 9H), 3.68 (q, J=11.2 Hz, 2H), 5.67 (s, 2H), 7.48 (dd, J=5.3, 1.8 Hz, 1H), 7.55 (d, J=1.7 Hz, 1H), 8.18 (d, J=1.7 Hz, 1H), 8.37 (d, J=5.4 Hz, 2H), 10.91 (s, 1H), 11.85 (s, 1H). LRMS: For C₂₁H₂₀F₃N₅O requires 415.42 found 416.2 (M+H).

### Example 167

### N-(4-Iodopyridin-2-yl)cyclopropanecarboxamide

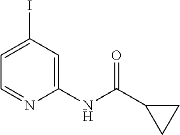 [SMILES: O=C(Nc1cc(I)ccn1)C1CC1]

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise cyclopropanecarbonyl chloride (285.07 mg, 0.247 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (30% EtOAc in petroleum ether) to give the titled product (622 mg, 2.16 mmol, 95%). ¹H NMR (500 MHz, DMSO-d₆) δ 10.90 (s, 1H), 8.54 (d, J=1.2 Hz, 1H), 8.04 (d, J=5.2 Hz, 1H), 7.49 (dd, J=5.2, 1.5 Hz, 1H), 2.00 (quin, J=6.2 Hz, 1H), 0.84 (s, 2H), 0.82 (s, 2H) ppm.

### N-(4-(3-Amino-7-bromo-1H-indazol-5-yl)pyridin-2-yl)cyclopropanecarboxamide

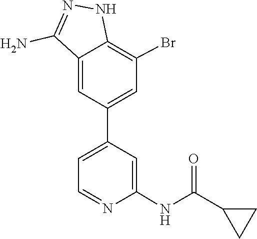 [SMILES: Nc1n[nH]c2c(Br)cc(-c3ccnc(NC(=O)C4CC4)c3)cc12]

To a solution of 7-bromo-5-(4,4,5,5-tetramethyl-1,3,2-dioxaborolan-2-yl)-1H-indazol-3-amine (796.30 mg, 2.35 mmol, 1.1 Eq) in a degassed 9:1 solution of dioxane/water (5 mL), were added N-(4-iodopyridin-2-yl)cyclopropanecarboxamide (617 mg, 2.14 mmol, 1 Eq), cesium carbonate (2.09 g, 6.42 mmol, 3 Eq) and bis(triphenylphosphine)palladium chloride (150.32 mg, 0.214 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (90% EtOAc in petroleum ether), to afford the titled compound (644 mg, 1.71 mmol, 80%). ¹H NMR (500 MHz, DMSO-d₆) δ 11.99 (br. s., 1H), 10.84 (s, 1H), 8.41 (s, 1H), 8.34 (d, J=5.2 Hz, 1H), 8.19 (s, 1H), 7.78 (s, 1H), 7.49 (dd, J=5.2, 1.5 Hz, 1H), 5.71 (br. s., 2H), 2.07-2.02 (m, 1H), 0.86-0.82 (m, 4H) ppm.

### N-(4-(3-Amino-7-(3,3-dimethylbut-1-yn-1-yl)-1H-indazol-5-yl)pyridin-2-yl)cyclopropane carboxamide

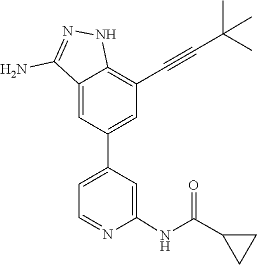 [SMILES: CC(C)(C)C#Cc1cc(-c2ccnc(NC(=O)C3CC3)c2)cc2c(N)n[nH]c12]

To a solution of N-(4-(3-amino-7-bromo-1H-indazol-5-yl)pyridin-2-yl)cyclopropanecarboxamide (200 mg, 0.53 mmol, 1 Eq) in a degassed 4:1 solution of DMF/triethylamine (2 mL) were added copper(I) iodide (20.46 mg, 0.11 mmol, 0.2 Eq), [1,1′-bis(diphenylphosphino)ferrocene]dichloropalladium(II) catalyst (39.31 mg, 0.053 mmol, 0.1 Eq) and 3,3-dimethylbut-1-yne (66.20 mg, 0.099 mL, 0.80 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (20 to 95% EtOAc in petroleum ether) followed by HPLC purification to give the titled product (152.40 mg, 0.40 mmol, 77%). ¹H NMR (500 MHz, DMSO-d₆) δ 11.83 (br. s, 1H), 10.84 (s, 1H), 8.41 (d, J=1.8 Hz, 1H), 8.34 (d, J=5.3 Hz, 1H), 8.15 (d, J=1.7 Hz, 1H), 7.52 (d, J=1.6 Hz, 1H), 7.40 (dd, J=5.3, 1.8 Hz, 1H), 5.65 (s, 2H), 2.08-2.03 (m, 1H), 1.38 (s, 9H), 0.88-0.81 (m, 4H) ppm. LRMS (ESI+ve): For C₂₂H₂₃N₅O Molecular Weight: 373.46 found 374.3 (M+H).

### Example 168

### tert-Butyl (4-iodopyridin-2-yl)carbamate

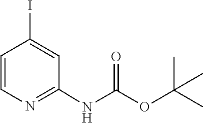

To a solution of 4-iodopyridin-2-amine (2.7 g 12.27 mmol) in dioxane (10 mL) was added DMAP (1.79 g, 14.72 mmol) under a blanket of argon followed by Boc₂O (2.812 g, 12.88 mmol). The resulting reaction mixture was stirred at 80° C. for 12 hours. The reaction was then cooled and quenched by pouring into water 20 mL, extracted with EtOAc (3×30 mL) and washed with brine (10 mL). The combined organic layers were concentrated and purified by flash eluting with a gradient of EtOAc:Petroleum Ether (0-50%) to afford the target compound as an off white solid (2.94 g, 75%). ¹H NMR (500 MHz, DMSO-d₆) δ 9.94 (s, 1H), 8.24 (d, J=1.4 Hz, 1H), 7.97 (d, J=5.2 Hz, 1H), 7.42 (dd, J=5.2, 1.5 Hz, 1H), 1.48 (s, 9H). LRMS (ESI+ve): For C₁₀H₁₃IN₂O₂ Molecular Weight: 320.1305 found 321.0

### N-(4-Iodopyridin-2-yl)isobutyramide

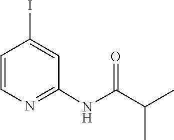

To a solution of tert-butyl (4-iodopyridin-2-yl)carbamate (0.5 g, 1.56 mmol) in dioxane (2 mL) was added NaHMDS (1.2 eq, 1M solution in THF) at 0° C. under a blanket of argon followed by isobutyryl chloride (0.183 g, 0.181 mL, 1.716 mmol) and the resulting reaction mixture allowed to warm up to room temperature and stirred at this temperature for 12 hours. The reaction was then quenched by pouring over ice/water 20 mL and extracted with EtOAc (3×30 mL) and washed with brine (10 mL). The combined organic layers were concentrated to afford the crude product which was taken up in dichloromethane (5 mL) and stirred at 0° C. with 2 ml TFA (1M_{aq}) for 3 hours, then quenched by pouring over ice/saturated NaHCO_{3Aq} 10 mL and extracted with EtOAc (3×30 mL), washed with brine (10 mL), concentrated and purified by flash column chromatography eluting with a gradient of EtOAc:Petroleum Ether (0-50%) to afford the target compound as a cream coloured solid (0.35 g, 78%). ¹H NMR (500 MHz, DMSO-d₆) δ 10.54 (s, 1H), 8.57 (d, J=1.5 Hz, 1H), 8.04 (d, J=5.2 Hz, 1H), 7.50 (dd, J=5.2, 1.5 Hz, 1H), 2.75 (h, J=6.8 Hz, 1H), 1.08 (d, J=6.8 Hz, 6H). LRMS (ESI+ve): For C₉H₁₁IN₂O Molecular Weight: 290.1045 found 291.0

### N-(4-(3-Amino-7-bromo-1H-indazol-5-yl)pyridin-2-yl)isobutyramide

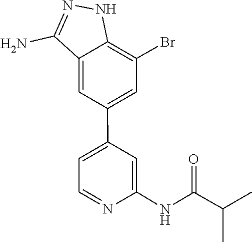 [SMILES: CC(C)C(=O)Nc1cc(-c2cc(Br)c3[nH]nc(N)c3c2)ccn1]

To a solution of N-(4-iodopyridin-2-yl)isobutyramide (035 g, 1.21 mmol) in a degassed solution of dioxane:H₂O (18:2 5 mL) was added cesium carbonate (1.18 g, 363 mmol), 7-bromo-5-(4,4,5,5-tetramethyl-1,3,2-dioxaborolan-2-yl)-1H-indazol-3-amine (0.488 g, 1.45 mmol) and bis(triphenylphosphine)palladium(II) dichloride catalyst (0.084 g, 0.121 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and the crude product used directly in the subsequent reaction.

### N-(4-(3-Amino-7-(3,3-dimethylbut-1-yn-1-yl)-1H-indazol-5-yl)pyridin-2-yl)isobutyramide

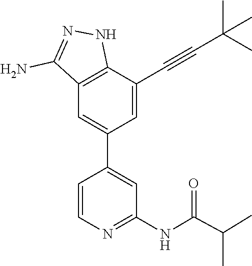 [SMILES: CC(C)C(=O)Nc1cc(-c2cc(C#CC(C)(C)C)c3[nH]nc(N)c3c2)ccn1]

To a solution of N-(4-(3-amino-7-bromo-1H-indazol-5-yl)pyridin-2-yl)isobutyramide in a degassed 4:1 solution of DMF:triethylamine (5 mL) was added [1,1′-bis(diphenylphosphino)ferrocene]dichloropalladium(II) catalyst (0.089 g, 0.121 mmol) and 3,3-dimethylbut-1-yne (0.099 g, 0.146 mL, 1.21 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether:EtOAc to afford the target compound as a yellow (0.035 g. 8% over two steps). ¹H NMR (500 MHz, DMSO-d₆) δ 11.91-11.81 (m, 1H), 10.47 (s, 1H), 8.45 (d, J=1.7 Hz, 1H), 8.33 (d, J=5.2 Hz, 1H), 8.17 (d, J=1.6 Hz, 1H), 7.96 (s, 1H), 7.54 (d, J=1.6 Hz, 1H), 7.40 (dd, J=5.2, 1.7 Hz, 1H), 5.66 (s, 2H), 2.80 (p, J=6.8 Hz, 1H), 1.38 (s, 9H), 1.12 (d, J=6.8 Hz, 6H). LRMS (ESI+ve): For C₂₂H₂₅N₅O Molecular Weight: 375.4760 found 376.3

### Example 169

### N-(4-Iodopyridin-2-yl)pivalamide

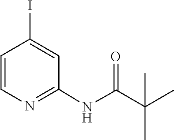

To a solution of tert-butyl (4-iodopyridin-2-yl)carbamate (0.5 g, 1.56 mmol) in dioxane (2 mL) was added NaHMDS (1.2 eq, 1M solution in THF) at 0° C. under a blanket of argon followed pivaloyl chloride 0.19 g, 0.188 mL, 1.72 mmol) and the resulting reaction mixture allowed to warm up to room temperature and stirred at this temperature for 12 hours. The reaction was then quenched by pouring over ice/water 20 mL and extracted with EtOAc (3×30 mL) and washed with brine (10 mL). The combined organic layers were concentrated to afford the crude product which was taken up in dichloromethane (5 mL) and stirred at 0° C. with 2 ml TFA (1M_{aq}) for 3 hours, then quenched by pouring over ice/saturated NaHCO_{3Aq} 10 mL and extracted with EtOAc (3×30 mL), washed with brine (10 mL), concentrated and purified by flash column chromatography eluting with a gradient of EtOAc:petroleum Ether (0-50%) to afford the target compound as a cream coloured solid 0.28 g, 60%). ¹H NMR (500 MHz, DMSO-d₆) δ 9.93 (s, 1H), 8.52 (d, J=1.7 Hz, 1H), 8.06 (d, J=5.2 Hz, 1H), 7.52 (dd, J=5.2, 1.5 Hz, 1H), 1.23 (s, 9H). LRMS (ESI+ve): For C₁₀H₁₃IN₂O Molecular Weight: 304.1315 found 305.0

### N-(4-(3-Amino-7-bromo-1H-indazol-5-yl)pyridin-2-yl)pivalamide

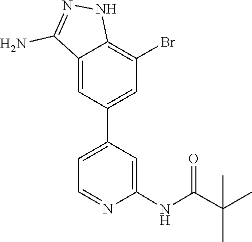 [SMILES: CC(C)(C)C(=O)Nc1cc(-c2cc(Br)c3[nH]nc(N)c3c2)ccn1]

To a solution of N-(4-iodopyridin-2-yl)pivalamide (0.28 g, 0.921 mmol) in a degassed solution of dioxane:H₂O (18:2 5 mL) was added cesium carbonate (0.899 g, 2.762 mmol), 7-bromo-5-(4,4,5,5-tetramethyl-1,3,2-dioxaborolan-2-yl)-1H-indazol-3-amine (0.373 g, 1.105 mmol) and bis(triphenylphosphine)palladium(II) dichloride catalyst (0.0646 g, 0.092 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and the crude product used directly in the subsequent reaction.

### N-(4-(3-Amino-7-(3,3-dimethylbut-1-yn-1-yl)-1H-indazol-5-yl)pyridin-2-yl)pivalamide

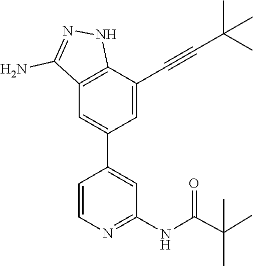 [SMILES: CC(C)(C)C#Cc1cc(-c2ccnc(NC(=O)C(C)(C)C)c2)cc2c(N)n[nH]c12]

To a solution of N-(4-(3-amino-7-bromo-1H-indazol-5-yl)pyridin-2-yl)pivalamide in a degassed 4:1 solution of DMF:triethylamine (5 mL) was added [1,1′-bis(diphenylphosphino)ferrocene]dichloropalladium(II) catalyst (0.0674 g, 0.092 mmol) copper iodide (0.035 g, 0.184 mmol) and 3,3-dimethylbut-1-yne (0.0756 g, 0.113 mL, 0.921 mmol), under an atmosphere of argon. The reaction was then sealed and heated at 90° C. for 18 hours.

The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether:EtOAc to afford the target compound as a yellow solid (0.014 g, 4% over two steps). ¹H NMR (500 MHz, DMSO-d₆) δ 10.10 (s, 1H), 8.37 (d, J=5.5 Hz, 1H), 8.34 (d, J=1.7 Hz, 1H), 8.26 (d, J=1.6 Hz, 1H), 7.62 (d, J=1.5 Hz, 1H), 7.54 (dd, J=5.4, 1.7 Hz, 1H), 1.38 (s, 9H), 1.29 (s, 9H). LRMS (ESI+ve): For C₂₃H₂₇N₅O Molecular Weight: 389.5030 found 390.1

### Example 170

### 2-Cyclopropyl-N-(4-iodopyridin-2-yl)acetamide

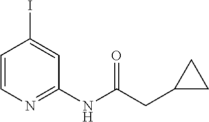 [SMILES: O=C(CC1CC1)Nc1cc(I)ccn1]

To a solution of 2-cyclopropylacetic acid (0.56 ml, 5.6 mmol) in 9.6 ml of THF cold down at 0° C. was added oxalyl chloride (0.54 ml, 6.4 mmol) followed by DMF (0.10 ml, 1.3 mmol) and was let run for 10 min. A solution of 4-iodopyridin-2-amine (400 mg, 1.8 mmol) in 4 ml of THF was added followed by triethylamine (0.93 ml, 6.7 mmol). The reaction mixture was run at RT for 2 hours. The resulting mixture was diluted with EtOAc, quenched with NaOH, extracted with EtOAc, washed with NaHCO₃, dry over MgSO₄, concentrated under reduced pressure and the residue was filtrated through silica (First column Petrol 40-60/EtOAc 88:12; Second column Toluene/EtOAc 9:1) to give the crude product as a yellow solid. (374 mg, 69%). ¹H NMR (400 MHz, (CD₃)₂CO): δ 0.21-0.29 (m, 2H), 0.52-0.58 (m, 2H), 1.10-1.19 (m, 1H), 2.39 (d, J=7.1 Hz, 2H), 7.48 (dd, J=5.2, 1.5 Hz, 1H), 7.99 (dd, J=5.2, 0.6 Hz, 1H), 8.74 (dd, J=1.5, 0.6 Hz, 1H), 9.35 (br s, 1H). LRMS: Calculated for C₁₀H₁₁IN₂O 302.0 found 303.1 (M+1).

### N-(4-(3-Amino-7-(3,3-dimethylbut-1-yn-1-yl)-1H-indazol-5-yl)pyridin-2-yl)-2-cyclopropylacetamide

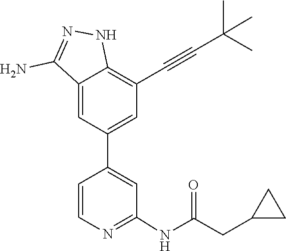 [SMILES: CC(C)(C)C#Cc1cc(-c2ccnc(NC(=O)CC3CC3)c2)cc2c(N)n[nH]c12]

A solution of 2-cyclopropyl-N-(4-iodopyridin-2-yl)acetamide (60 mg, 0.20 mmol), Cs₂CO₃ (200 mg, 0.60 mmol), bis(triphenylphosphine) palladium(II) chloride (25 mg, 0.04 mmol) and 7-(3,3-dimethylbut-1-yn-1-yl)-5-(4,4,5,5-tetramethyl-1,3,2-dioxaborolan-2-yl)-1H-indazol-3-amine (80 mg, 0.23 mmol) in 1 ml of dioxane-water (4:1) was degassed in sealed tube and submitted to reaction. The reaction mixture was heated to 80° C. for 5 hours. The resulting mixture was diluted with EtOAc, filtered through silica, concentrated under reduced pressure and the residue was purified by HPLC to afford the title compound as a yellow solid. (11 mg, 14%). ¹H NMR (400 MHz, DMSO-d₆): δ 0.16-0.26 (m, 2H), 0.44-0.55 (m, 2H), 1.00-1.15 (m, 1H), 1.37 (s, 9H), 2.33 (d, J=7.0 Hz, 2H), 7.45 (dd, J=5.4, 1.8 Hz, 1H), 7.59 (d, J=1.7 Hz, 1H), 8.22 (d, J=1.7 Hz, 1H), 8.33 (d, J=5.4 Hz, 1H), 8.38 (app s, 1H), 10.56 (br s, 1H), 3 protons missing. LRMS: Calculated for C₁₉H₂₁N₅O₂S 387.2 found 388.1 (M+1).

### Example 171

### N-(4-Iodopyridin-2-yl)-3-methylbutanamide

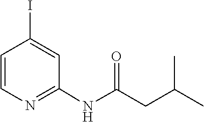

To a solution of tert-butyl (4-iodopyridin-2-yl)carbamate (0.5 g, 1.56 mmol) in dioxane (2 mL) was added NaHMDS (1.2 eq, 1M solution in THF) at 0° C. under a blanket of argon followed by 3-methylbutyryl chloride (0.207 g, 0.213 mL 1.716 mmol) and the resulting reaction mixture allowed to warm up to room temperature and stirred at this temperature for 12 hours. The reaction was then quenched by pouring over ice/water 20 mL and extracted with EtOAc (3×30 mL) and washed with brine (10 mL). The combined organic layers were concentrated to afford the crude product which was taken up in dichloromethane (5 mL) and stirred at 0° C. with 2 ml TFA (1M_{aq}) for 3 hours, then quenched by pouring over ice/saturated NaHCO_{3Aq} 10 mL and extracted with EtOAc (3×30 mL), washed with brine (10 mL), concentrated and purified by flash column chromatography eluting with a gradient of EtOAc:Petroleum Ether (0-50%) to afford the target compound as a cream coloured solid 0.32 g, 68%). ¹H NMR (500 MHz, DMSO-d₆) δ 10.54 (s, 1H), 8.57 (d, J=1.5 Hz, 1H), 8.03 (d, J=5.2 Hz, 1H), 7.50 (dd, J=5.2, 1.5 Hz, 1H), 2.28 (d, J=7.1 Hz, 2H), 2.07 (dt, J=13.6, 6.9 Hz, 1H), 0.92 (d, J=6.7 Hz, 6H). LRMS (ESI+ve): For C₁₀H₁₃IN₂O Molecular Weight: 304.1315 found 305.0

### N-(4-(3-Amino-7-bromo-1H-indazol-5-yl)pyridin-2-yl)-3-methylbutanamide

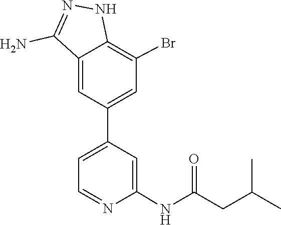 [SMILES: CC(C)CC(=O)Nc1cc(-c2cc(Br)c3[nH]nc(N)c3c2)ccn1]

To a solution of N-(4-iodopyridin-2-yl)-3-methylbutanamide (0.32 g, 1.052 mmol) in a degassed solution of dioxane:H₂O (18:2 5 mL) was added cesium carbonate (0.977 g, 3.156 mmol), 7-bromo-5-(4,4,5,5-tetramethyl-1,3,2-dioxaborolan-2-yl)-1H-indazol-3-amine (0.4255 g, 1.262 mmol) and bis(triphenylphosphine)palladium(II) dichloride catalyst (0.074 g, 0.1052 mmol) under an atmosphere of argon. The reaction was then sealed and stirred at 90° C. for 18 hours.

The cooled reaction was then diluted with EtOAc (30 mL) and washed with water (30 mL) and brine (10 mL). The organic layer was then concentrated under reduced pressure and the crude product used directly in the subsequent reaction.

### N-(4-(3-Amino-7-(3,3-dimethylbut-1-yn-1-yl)-1H-indazol-5-yl)pyridine-2-yl)-3-methylbutanamide

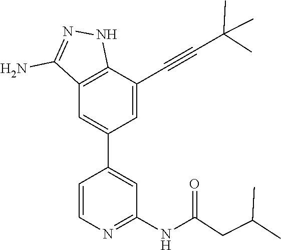 [SMILES: CC(C)CC(=O)Nc1cc(-c2cc(C#CC(C)(C)C)c3[nH]nc(N)c3c2)ccn1]

To a solution of N-(4-(3-amino-7-bromo-1H-indazol-5-yl)pyridin-2-yl)-3-methylbutanamide in a degassed 4:1 solution of DMF:triethylamine (5 mL) was added [1,1′-bis(diphenylphosphino)ferrocene]dichloropalladium(II) catalyst (0.07697 g, 0.1052 mmol) and 3,3-dimethylbut-1-yne (0.0864 g, 0.129 mL, 1.052 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether:EtOAc to afford the target compound as a yellow (0.013 g. 3% over two steps). ¹H NMR (400 MHz, DMSO-d₆) δ 11.84 (s, 1H), 10.46 (s, 1H), 8.43 (s, 1H), 8.32 (d, J=5.3 Hz, 1H), 8.16 (d, J=1.7 Hz, 1H), 7.53 (d, J=1.7 Hz, 1H), 7.40 (dd, J=5.3, 1.7 Hz, 1H), 5.66 (s, 2H), 2.32 (d, J=7.2 Hz, 2H), 2.11 (dt, J=13.5, 6.8 Hz, 1H), 1.38 (s, 9H), 0.95 (d, J=6.5 Hz, 6H). LRMS (ESI+ve): For C₂₃H₂₇N₅O Molecular Weight: 389.5030 found 390.3

### Example 172

### N-(4-Iodopyridin-2-yl)cyclobutanecarboxamide

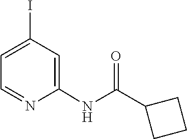 [SMILES: O=C(Nc1cc(I)ccn1)C1CCC1]

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise cyclobutanecarbonyl chloride (322.95 mg, 0.310 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (30% EtOAc in petroleum ether) to give the titled product (583 mg, 1.93 mmol, 85%). ¹H NMR (500 MHz, DMSO-d₆) δ ¹H NMR (500 MHz, DMSO-d₆) 10.41 (s, 1H), 8.58 (s, 1H), 8.01 (d, J=5.2 Hz, 1H), 7.48 (dd, J=5.2 Hz, 1.37 Hz, 1H), 3.38-3.32 (m, 1H), 2.23-2.16 (m, 2H), 2.12-2.06 (m, 2H), 1.96-1.87 (m, 1H), 1.83-1.76 (m, 1H) ppm.

### N-(4-(3-Amino-7-bromo-1H-indazol-5-yl)pyridin-2-yl)cyclobutanecarboxamide

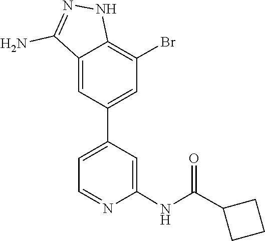 [SMILES: Nc1n[nH]c2c(Br)cc(-c3ccnc(NC(=O)C4CCC4)c3)cc12]

To a solution of 7-bromo-5-(4,4,5,5-tetramethyl-1,3,2-dioxaborolan-2-yl)-1H-indazol-3-amine (615.35 mg, 1.82 mmol, 1.1 Eq) in a degassed 9:1 solution of dioxane/water (5 mL), were added N-(4-iodopyridin-2-yl)cyclopropanecarboxamide (500 mg, 1.65 mmol, 1 Eq), cesium carbonate (1.61 g, 4.95 mmol, 3 Eq) and bis(triphenylphosphine)palladium chloride (115.81 mg, 0.165 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (10% to 90% EtOAc in petroleum ether), to afford the titled compound (573.58 mg, 1.48 mmol, 90%). ¹H NMR (500 MHz, DMSO-d₆) δ 12.02 (br. s., 1H), 10.37 (s, 1H), 8.48 (s, 1H), 8.34 (d, J=5.0 Hz, 1H), 8.23 (s, 1H), 7.82 (s, 1H), 7.42-7.39 (m, 1H), 5.74 (br. s., 2H), 3.42 (quin, J=8.3 Hz, 1H), 2.30-2.21 (m, 2H), 2.16-2.10 (m, 2H), 1.98-1.92 (m, 1H), 1.87-1.79 (m, 1H) ppm.

### N-(4-(3-Amino-7-(3,3-dimethylbut-1-yn-1-yl)-1H-indazol-5-yl)pyridin-2-yl)cyclobutanecarboxamide

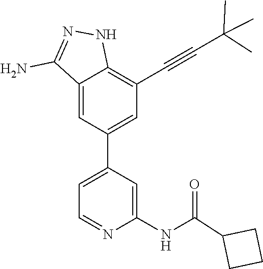 [SMILES: CC(C)(C)C#Cc1cc(-c2ccnc(NC(=O)C3CCC3)c2)cc2c(N)n[nH]c12]

To a solution of N-(4-(3-amino-7-bromo-1H-indazol-5-yl)pyridin-2-yl)cyclobutanecarboxamide (300 mg, 0.776 mmol, 1 Eq) in a degassed 4:1 solution of DMF/triethylamine (2 mL) were added copper(I) iodide (29.58 mg, 0.155 mmol, 0.2 Eq), [1,1′-bis(diphenylphosphino)ferrocene]dichloropalladium(II) catalyst (56.34 mg, 0.077 mmol, 0.1 Eq) and 3,3-dimethylbut-1-yne (95.61 mg, 0.143 mL, 1.164 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (10 to 100% EtOAc in petroleum ether) followed by HPLC purification to give the titled product (150.34 mg, 0.388 mmol, 50%). ¹H NMR (500 MHz, DMSO-d₆) δ 11.84 (s, 1H), 10.33 (s, 1H), 8.45 (s, 1H), 8.31 (d, J=5.2 Hz, 1H), 8.16 (s, 1H), 7.53 (d, J=1.5 Hz, 1H), 7.39 (d, J=5.2 Hz, 1H), 5.66 (br. s, 2H), 3.41 (q, J=8.3 Hz, 2H), 2.24 (dq, J=11.1, 8.7 Hz, 2H), 2.12 (tq, J=9.3, 4.8 Hz, 2H), 1.94 (h, J=9.0 Hz, 1H), 1.83 (dd, J=12.1, 7.5 Hz, 1H), 1.37 (s, 9H) ppm. LRMS (ESI+ve): For C₂₃H₂₅N₅O requires 387.49 found 388.2 (M+H).

### Example 173

### N-(4-Iodopyridin-2-yl)cyclopentanecarboxamide

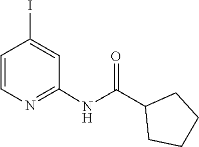 [SMILES: O=C(Nc1cc(I)ccn1)C1CCCC1]

To a solution of tert-butyl (4-iodopyridin-2-yl)carbamate (0.5 g, 1.56 mmol) in dioxane (2 mL) was added NaHMDS (1.2 eq, 1M solution in THF) at 0° C. under a blanket of argon followed by cyclopentanoyl chloride (0.2275 g, 1.716 mmol) and the resulting reaction mixture allowed to warm up to room temperature and stirred at this temperature for 12 hours. The reaction was then quenched by pouring over ice/water 20 mL and extracted with EtOAc (3×30 mL) and washed with brine (10 mL). The combined organic layers were concentrated to afford the crude product which was taken up in dichloromethane (5 mL) and stirred at 0° C. with 2 ml TFA (1M_{aq}) for 3 hours, then quenched by pouring over ice/saturated NaHCO₃ 10 mL and extracted with EtOAc (3×30 mL), washed with brine (10 mL), concentrated and purified by flash column chromatography eluting with a gradient of EtOAc:Petroleum Ether (0-50%) to afford the target compound as a cream coloured solid (0.29 g, 58%). ¹H NMR (500 MHz, DMSO-d₆) δ 10.55 (s, 1H), 8.57 (d, J=1.4 Hz, 1H), 8.03 (d, J=5.2 Hz, 1H), 7.49 (dd, J=5.2, 1.5 Hz, 1H), 2.92 (q, J=7.7 Hz, 1H), 1.84 (ddt, J=13.4, 5.6, 3.0 Hz, 2H), 1.74-1.62 (m, 5H), 1.57-1.49 (m, 2H). LRMS (ESI+ve): For C₁₁H₁₃IN₂O Molecular Weight: 316.1425 found 317.0

### N-(4-(3-Amino-7-bromo-1H-indazol-5-yl)pyridin-2-yl)cyclopentanecarboxamide

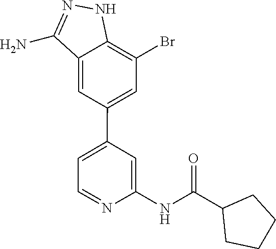 [SMILES: Nc1n[nH]c2c(Br)cc(-c3ccnc(NC(=O)C4CCCC4)c3)cc12]

To a solution of N-(4-iodopyridin-2-yl)cyclopentanecarboxamide (0.29 g, 0.917 mmol) in a degassed solution of dioxane:H₂O (18:2 5 mL) was added cesium carbonate (0.896 g, 2.751 mmol), 7-bromo-5-(4,4,5,5-tetramethyl-1,3,2-dioxaborolan-2-yl)-1H-indazol-3-amine (0.370 g, 1.10 mmol) and bis(triphenylphosphine)palladium(II) dichloride catalyst (0.077 g, 0.110 mmol) under an atmosphere of argon. The reaction was then sealed and stirred at 90° C. for 18 hours.

The cooled reaction was then diluted with EtOAc (30 mL) and washed with water (30 mL) and brine (10 mL). The organic layer was then concentrated under reduced pressure and the crude product used directly in the subsequent reaction.

### N-(4-(3-Amino-7-(3,3-dimethylbut-1-yn-1-yl)-1H-indazol-5-yl)pyridin-2-yl)cyclopentanecarboxamide

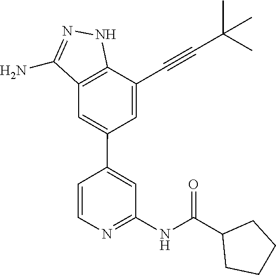 [SMILES: CC(C)(C)C#Cc1cc(-c2ccnc(NC(=O)C3CCCC3)c2)cc2c(N)n[nH]c12]

To a solution of N-(4-(3-amino-7-bromo-1H-indazol-5-yl)pyridin-2-yl)cyclopentanecarboxamide in a degassed 4:1 solution of DMF:triethylamine (5 mL) was added [1,1′-bis(diphenylphosphino)ferrocene]dichloropalladium(II) catalyst (0.067 g, 0.0917 mmol) copper iodide (0.0349 g, 0.1834 mmol) and 3,3-dimethylbut-1-yne (0.0753 g, 0.917 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether:EtOAc to afford the target compound as a yellow solid (0.018 g. 5%). ¹H NMR (500 MHz, DMSO-d₆) δ 10.64 (s, 1H), 8.38 (d, J=1.8 Hz, 1H), 8.34 (d, J=5.4 Hz, 1H), 8.22 (d, J=1.7 Hz, 1H), 7.59 (d, J=1.6 Hz, 1H), 7.46 (dd, J=5.4, 1.7 Hz, 1H), 2.99 (p, J=7.9 Hz, 1H), 1.94-1.84 (m, 2H), 1.80-1.65 (m, 4H), 1.59 (ddt, J=11.8, 8.9, 3.9 Hz, 2H), 1.38 (s, 9H). LRMS (ESI+ve): For C₂₄H₂₇N₅O Molecular Weight: 401.5140 found 402.3.

### Example 174

### 2-((4-Iodopyridin-2-yl)amino)-2-oxoethyl acetate

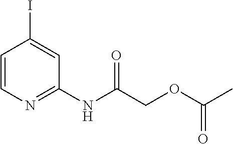

A solution of 2-chloro-2-oxoethyl acetate (0.62 g, 0.5 mL, 4.55 mmol, 1 Eq) in anhydrous DCM (1 mL) was added dropwise to a solution of 4-iodopyridin-2-amine (1 g, 4.55 mmol, 1 Eq) and triethylamine (1.38 g, 2 mL, 13.65 mmol, 2 Eq) in Dioxane (5 mL) at 5° C. The reaction mixture was allowed to stir at rt for 6 h. The reaction was extracted between EtOAc (10 mL) and saturated solution of NaHCO₃ (5 mL). The organic layer was washed with brine (2×5 mL), dried over anhydrous Na₂SO₄ and evaporated under reduced pressure. The crude residue was purified using column chromatography (20% EtOAc in petroleum ether) to give the titled product as white crystals (0.8 g, 2.5 mmol, 55%).

### 2-((4-(3-Amino-7-bromo-1H-indazol-5-yl)pyridin-2-yl)amino)-2-oxoethyl acetate

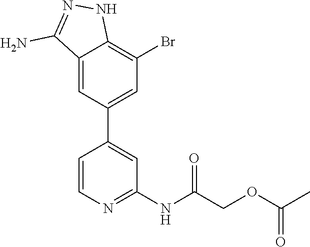 [SMILES: CC(=O)OCC(=O)Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (1 g, 2.96 mmol, 1 Eq), 2-((4-iodopyridin-2-yl)amino)-2-oxoethyl acetate (0.95 g, 2.96 mmol, 1 Eq), cesium carbonate (2.9 g, 8.88 mmol, 3 Eq) and bis(triphenylphosphine)palladium chloride (312 mg, 0.44 mmol, 0.15 Eq) in dioxane (8 mL) and water (2 mL) was stirred under nitrogen atmosphere at 80° C. for 18 h. Cold water (10 mL) was added to the reaction mixture. The produced precipitate was filtered and washed with cold water (2×5 mL). The crude product was dried and purified using column chromatography (90% EtOAc in petroleum ether) to give the titled product as brown solid (0.7 g, 1.73 mmol, 58%).

### 2-((4-(3-Amino-7-(3,3-dimethylbut-1-yn-1-yl)-1H-indazol-5-yl)pyridin-2-yl)amino)-2-oxoethyl acetate

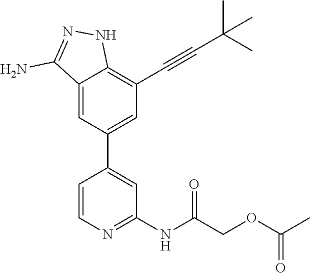 [SMILES: CC(=O)OCC(=O)Nc1cc(-c2cc(C#CC(C)(C)C)c3[nH]nc(N)c3c2)ccn1]

3,3-Dimethylbut-1-yne (204 mg, 0.3 mL, 2.48 mmol, 2 Eq) was added to a solution of 2-((4-(3-amino-7-bromo-1H-indazol-5-yl)pyridin-2-yl)amino)-2-oxoethyl acetate (0.5 g, 1.24 mmol, 1 Eq), copper(I) iodide (47 mg, 0.24 mmol, 0.2 Eq) and [1,1′-bis(diphenylphosphino)ferrocene]dichloropalladium(II) (91 mg, 0.13 mmol, 0.1 Eq) in anhydrous DMF (2 mL) and triethylamine (2 mL) under nitrogen atmosphere. The reaction mixture was allowed to stir at 60° C. for 18 h. The reaction mixture was extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) to give the titled product as green solid (0.2 g, 0.49 mmol, 40%).

### N-(4-(3-Amino-7-(3,3-dimethylbut-1-yn-1-yl)-1H-indazol-5-yl)pyridin-2-yl)-2-hydroxyacetamide

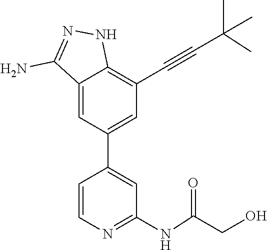 [SMILES: CC(C)(C)C#Cc1cc(-c2ccnc(NC(=O)CO)c2)cc2c(N)n[nH]c12]

A solution of K₂CO₃ (138 mg, 1 mmol, 4 Eq) in water (0.5 mL) was added to a solution of 2-((4-(3-amino-7-(3,3-dimethylbut-1-yn-1-yl)-1H-indazol-5-yl)pyridin-2-yl)amino)-2-oxoethyl acetate (100 mg, 0.25 mmol, 1 Eq) in MeOH (2 mL). The reaction mixture was stirred at rt for 1 h. The produced precipitate was filtered, washed with water (2×1 mL), dried and purified using HPLC (60% MeCN in H₂O) to give the titled product as white solid (50 mg, 0.14 mmol, 56%). ¹H NMR (500 MHz, DMSO) δ 1.38 (s, 9H), 4.09 (d, J=5.9 Hz, 2H), 5.66 (s, 2H), 5.82 (t, J=5.9 Hz, 1H), 7.46 (dd, J=5.3, 1.7 Hz, 1H), 7.55 (d, J=1.7 Hz, 1H), 8.18 (d, J=1.7 Hz, 1H), 8.35 (d, J=5.3 Hz, 1H), 8.42 (d, J=1.7 Hz, 1H), 9.70 (s, 1H), 11.86 (s, 1H). LRMS: For C₂₀H₂₁N₅O₂ requires 363.42 found 364.3 (M+H).

### Example 175

### N-(4-Iodopyridin-2-yl)-2-methoxyacetamide

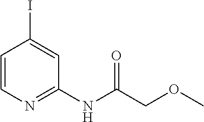

A solution of 2-methoxyacetyl chloride (0.49 g, 0.4 mL, 4.55 mmol, 1 Eq) in anhydrous DCM (1 mL) was added dropwise to a solution of 4-iodopyridin-2-amine (1 g, 4.55 mmol, 1 Eq) and triethylamine (1.38 g, 2 mL, 13.65 mmol, 2 Eq) in Dioxane (5 mL) at 5° C. The reaction mixture was allowed to stir at rt for 6 h. The reaction was extracted between EtOAc (10 mL) and saturated solution of NaHCO₃ (5 mL). The organic layer was washed with brine (2×5 mL), dried over anhydrous Na₂SO₄ and evaporated under reduced pressure. The crude residue was purified using column chromatography (20% EtOAc in petroleum ether) to give the titled product as white crystals (0.9 g, 3.1 mmol, 68%). ¹H NMR (500 MHz, DMSO) δ 3.37 (s, 3H), 4.07 (s, 2H), 7.54 (dd, J=5.2, 1.5 Hz, 1H), 8.05 (d, J=5.2 Hz, 1H), 8.52 (d, J=1.5 Hz, 1H), 10.15 (s, 1H).

### N-(4-(3-Amino-7-bromo-1H-indazol-5-yl)pyridin-2-yl)-2-methoxyacetamide

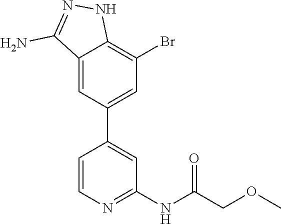 [SMILES: COCC(=O)Nc1cc(-c2cc(Br)c3[nH]nc(N)c3c2)ccn1]

A solution of 7-bromo-5-(4,4,5,5-tetramethyl-1,3,2-dioxaborolan-2-yl)-1 n (1 g, 2.96 mmol, 1 Eq), N-(4-iodopyridin-2-yl)-2-methoxyacetamide (0.9 g, 2.96 mmol, 1 Eq), cesium carbonate (2.9 g, 8.88 mmol, 3 Eq) and bis(triphenylphosphine)palladium chloride (312 mg, 0.44 mmol, 0.15 Eq) in dioxane (8 mL) and water (2 mL) was stirred under nitrogen atmosphere at 70° C. for 18 h. Cold water (10 mL) was added to the reaction mixture. The produced precipitate was filtered and washed with cold water (2×5 mL). The crude product was dried and purified using column chromatography (95% EtOAc in petroleum ether) to give the titled product as yellow solid (0.4 g, 1.1 mmol, 37%).

### N-(4-(3-Amino-7-(3,3-dimethylbut-1-yn-1-yl)-1H-indazol-5-yl)pyridin-2-yl)-2-methoxyacetamide

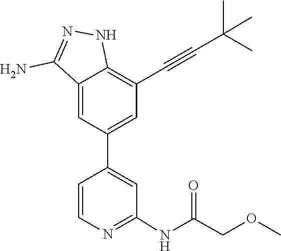 [SMILES: COCC(=O)Nc1cc(-c2cc(C#CC(C)(C)C)c3[nH]nc(N)c3c2)ccn1]

3,3-Dimethylbut-1-yne (204 mg, 0.3 mL, 2.48 mmol, 2 Eq) was added to a solution of N-(4-(3-amino-7-bromo-1H-indazol-5-yl)pyridin-2-yl)-2-methoxyacetamide (0.47 g, 1.24 mmol, 1 Eq), copper(I) iodide (47 mg, 0.24 mmol, 0.2 Eq) and [1,1′-bis(diphenylphosphino)ferrocene]dichloropalladium(II) (91 mg, 0.13 mmol, 0.1 Eq) in anhydrous DMF (2 mL) and triethylamine (2 mL) under nitrogen atmosphere. The reaction mixture was allowed to stir at 60° C. for 18 h. The reaction mixture was extracted between EtOAc (10 mL) and water (5 mL). The organic layer was washed with brine (2×5 mL), dried over anhydrous Na₂SO₄ and evaporated under reduced pressure. The crude residue was purified using column chromatography (95% EtOAc in petroleum ether) followed by HPLC purification (60% MeCN in H₂O) to give the titled product as white solid (0.25 g, 0.66 mmol, 53%). ¹H NMR (500 MHz, DMSO) δ 1.38 (s, 9H), 3.41 (s, 3H), 4.12 (s, 2H), 5.67 (s, 2H), 7.45 (dd, J=5.3, 1.7 Hz, 1H), 7.55 (d, J=1.6 Hz, 1H), 8.19 (d, J=1.6 Hz, 1H), 8.35 (d, J=5.3 Hz, 1H), 8.40 (s, 1H), 10.03 (s, 1H), 11.86 (s, 1H). LRMS: For C₂₁H₂₃N₅O₂ requires 377.45 found 378.4 (M+H).

### Example 176

### 5-(2-(Cyclopropylamino)pyridine-4-yl)-7-((3-methyloxetan-3-yl)ethynyl)-1H-indazol-3-amine

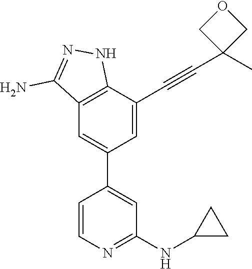 [SMILES: CC1(C#Cc2cc(-c3ccnc(NC4CC4)c3)cc3c(N)n[nH]c23)COC1]

To a solution of 7-bromo-5-(2-(cyclopropylamino)pyridin-4-yl)-1H-indazol-3-amine (0.100 g, 0.290 mmol) in a degassed 4:1 solution of DMF:triethylamine (5 mL) was added [1,1′-bis(diphenylphosphino)ferrocene]dichloropalladium(II) catalyst (0.015 g, 0.010 mmol) and 3-ethynyl-3-methyloxetane (0.0341 g, 0.348 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc to afford the target compound as a cream coloured solid (0.028 g, 27%). ¹H NMR (500 MHz, DMSO-d₆) δ 11.93 (s, 1H), 8.14 (d, J=1.6 Hz, 1H), 8.04 (d, J=5.3 Hz, 1H), 7.60 (d, J=1.6 Hz, 1H), 6.88 (dd, J=5.3, 1.7 Hz, 1H), 6.85 (d, J=1.6 Hz, 1H), 6.77 (d, J=2.5 Hz, 1H), 5.63 (s, 2H), 4.92-4.90 (m, 2H), 4.46 (d, J=5.3 Hz, 2H), 2.60 (dh, J=6.5, 3.3 Hz, 1H), 1.72 (s, 3H), 0.75 (td, J=6.7, 4.6 Hz, 2H), 0.48-0.45 (m, 2H). LRMS (ESI+ve): For C₂₁H₂₁N₅O Molecular Weight: 359.4330 found 360.3

### Example 177

### N-(4-(3-Amino-7-((3-methyloxetan-3-yl)ethynyl)-1H-indazol-5-yl)pyridin-2-yl)acetamide

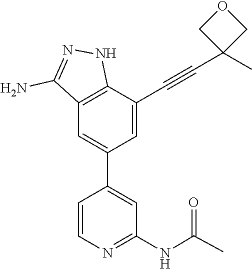 [SMILES: CC(=O)Nc1cc(-c2cc(C#CC3(C)COC3)c3[nH]nc(N)c3c2)ccn1]

To a solution of N-(4-(3-amino-7-bromo-1H-indazol-5-yl)pyridin-2-yl)acetamide (0.1 g, 0.288 mmol) in a degassed 4:1 solution of DMF:triethylamine (5 mL) was added [1,1′-bis(diphenylphosphino)ferrocene]dichloropalladium(II) catalyst (0.021 g, 0.0288 mmol) Copper Iodide (0.011 g, 0.0576 mmol), and 3-ethynyl-3-methyloxetane (0.033 g, 0.3456 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a dark brown solid (0.025 g. 24%). ¹H NMR (500 MHz, DMSO-d₆) δ 11.99 (s, 1H), 10.53 (s, 1H), 8.40 (s, 1H), 8.33 (d, J=5.3 Hz, 1H), 8.20 (d, J=1.6 Hz, 1H), 7.61 (d, J=1.6 Hz, 1H), 7.40 (dd, J=5.3, 1.7 Hz, 1H), 5.68 (s, 2H), 4.92 (d, J=5.4 Hz, 2H), 4.45 (d, J=5.4 Hz, 2H), 2.14 (s, 3H), 1.72 (s, 3H). LRMS (ESI+ve): For C₂₀H₁₉N₅O₂ Molecular Weight: 361.4050 found 362.2

### Example 178

### N-(4-(3-Amino-7-(phenylethynyl)-1H-indazol-5-yl)pyridin-2-yl)acetamide

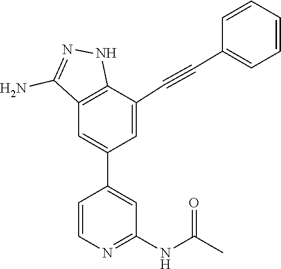 [SMILES: CC(=O)Nc1cc(-c2cc(C#Cc3ccccc3)c3[nH]nc(N)c3c2)ccn1]

To a solution of N-(4-(3-amino-7-bromo-1H-indazol-5-yl)pyridin-2-yl)acetamide (0.1 g, 0.289 mmol) in a degassed 4:1 solution of DMF:triethylamine (5 mL) was added [1,1′-bis(diphenylphosphino)ferrocene]dichloropalladium(II) catalyst (0.0395 g, 0.0289 mmol), copper iodide (0.015 g, 0.0789 mmol) and ethynylbenzene (0.0354 g, 0.038 mL, 0.346 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc to afford the target compound as a yellow (0.063 g, 57%). ¹H NMR (500 MHz, DMSO-d₆) δ 12.15 (s, 1H), 10.55 (s, 1H), 8.43 (s, 1H), 8.36 (d, J=5.2 Hz, 1H), 8.25 (d, J=1.5 Hz, 1H), 7.76 (dd, J=5.8, 1.9 Hz, 3H), 7.52-7.43 (m, 4H), 5.73 (s, 2H), 2.15 (s, 3H). LRMS (ESI+ve): For C₂₂H₁₇N₅O Molecular Weight: 367.4120 found 368.3.

### Example 179

### Methyl (4-iodopyridin-2-yl)carbamate

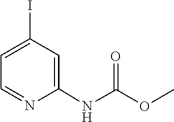

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise methyl chloroformate (257.71 mg, 0.21 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (20% EtOAc in petroleum ether) to give the titled product as a white solid (380 mg, 60%). ¹H NMR (500 MHz, DMSO-d₆) δ 10.33 (s, 1H), 8.26 (d, J=1.4 Hz, 1H), 7.98 (d, J=5.2 Hz, 1H), 7.45 (dd, J=5.2, 1.5 Hz, 1H), 3.68 (s, 3H) ppm.

### Methyl (4-(3-amino-7-bromo-1H-indazol-5-yl)pyridin-2-yl)carbamate

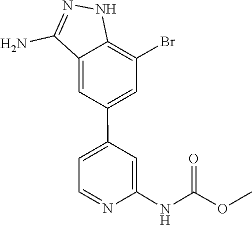 [SMILES: COC(=O)Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (508.14 mg, 1.50 mmol, 1.1 Eq) in a degassed 9:1 solution of dioxane/water (5 mL), were added methyl (4-iodopyridin-2-yl)carbamate (380 mg, 1.36 mmol, 1 Eq), cesium carbonate (1.33 g, 4.08 mmol, 3 Eq) and bis(triphenylphosphine)palladium chloride (95.46 mg, 0.136 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (90% EtOAc in petroleum ether), to afford the titled compound as a green solid (404 mg, 1.115 mmol, 82%). ¹H NMR (500 MHz, DMSO-d₆) δ 12.00 (s, 1H), 10.23 (s, 1H), 8.30 (d, J=5.3 Hz, 1H), 8.20 (d, J=1.5 Hz, 1H), 8.14 (d, J=1.7 Hz, 1H), 7.79 (d, J=1.5 Hz, 1H), 7.36 (dd, J=5.3, 1.7 Hz, 1H), 5.72 (s, 2H), 3.71 (s, 3H) ppm. LRMS (ESI+ve): For C₁₄H₁₂BrN₅O₂ requires 362.19 found 362.1, 363.0, 364.0, 365.0 (M+H).

### Methyl (4-(3-amino-7-(cyclopropylethynyl)-1H-indazol-5yl)-pyridin-2-yl)carbamate

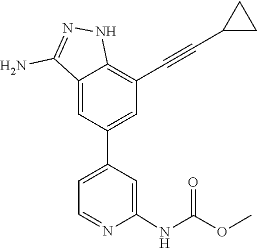 [SMILES: COC(=O)Nc1cc(-c2cc(C#CC3CC3)c3[nH]nc(N)c3c2)ccn1]

To a solution of methyl (4-(3-amino-7-bromo-1H-indazol-5-yl)pyridin-2-yl)carbamate (128.6 mg, 0.355 mmol, 1 Eq) in a degassed 4:1 solution of DMF/triethylamine (3 mL) were added copper(I) iodide (13.52 mg, 0.071 mmol, 0.2 Eq), [1,1′-bis(diphenylphosphino)ferrocene]dichloropalladium(II) catalyst (25.97 mg, 0.035 mmol, 0.1 Eq) and ethynylcyclopropane (35.19 mg, 0.045 mL, 0.532 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a solid (73.99 mg, 0.213 mmol, 60%). ¹H NMR (500 MHz, DMSO-d₆) δ 11.87 (s, 1H), 10.20 (s, 1H), 8.28 (d, J=5.3 Hz, 1H), 8.13 (t, J=1.6 Hz, 2H), 7.56 (d, J=1.7 Hz, 1H), 7.34 (dd, J=5.3, 1.7 Hz, 1H), 5.62 (s, 2H), 3.70 (s, 3H), 1.65-1.58 (m, 1H), 0.93-0.90 (m, 4H) ppm. LRMS (ESI+ve): For C₁₉H₁₇N₅O₂ requires 347.38 found 348.1 (M+H).

### Example 180

### Methyl (4-(3-amino-7-(3-hydroxy-3-methylbut-1-yn-1-yl)-1H-indazol-5-yl)pyridin-2-yl)carbamate

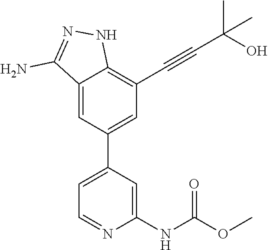 [SMILES: COC(=O)Nc1cc(-c2cc(C#CC(C)(C)O)c3[nH]nc(N)c3c2)ccn1]

To a solution of methyl (4-(3-amino-7-bromo-1H-indazol-5-yl)pyridin-2-yl)carbamate (200 mg, 0.552 mmol, 1 Eq) in a degassed 4:1 solution of DMF/triethylamine (5 mL) were added copper(I) iodide (21 mg, 0.11 mmol, 0.2 Eq), [1,1′-bis(diphenylphosphino)ferrocene]dichloropalladium(II) catalyst (40.4 mg, 0.055 mmol, 0.1 Eq) and 2-methylbut-3-yn-2-ol (69.64 mg, 0.080 mL, 0.828 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a solid (147.23 mg, 0.402 mmol, 73%). ¹H NMR (500 MHz, DMSO-d₆) δ 11.84 (s, 1H), 10.22 (s, 1H), 8.29 (d, J=5.3 Hz, 1H), 8.19 (d, J=1.6 Hz, 1H), 8.14 (d, J=1.7 Hz, 1H), 7.57 (d, J=1.6 Hz, 1H), 7.36 (dd, J=5.3, 1.7 Hz, 1H), 5.67 (s, 2H), 5.46 (s, 1H), 3.71 (s, 3H), 1.54 (s, 6H) ppm. LRMS (ESI+ve): For C₁₉H₁₉N₅O₃ requires 365.39 found 366.2 (M+H).

### Example 181

### Methyl (4-(3-amino-7-(3-amino-3-methylbut-1-yn-1-yl)-1H-indazol-5-yl)pyridin-2-yl)carbamate1

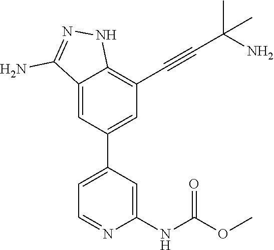 [SMILES: COC(=O)Nc1cc(-c2cc(C#CC(C)(C)N)c3[nH]nc(N)c3c2)ccn1]

To a solution of methyl (4-(3-amino-7-bromo-1H-indazol-5-yl)pyridin-2-yl)carbamate (219 mg, 0.605 mmol, 1 Eq) in a degassed 4:1 solution of DMF/triethylamine (5 mL) were added copper(I) iodide (23.04 mg, 0.121 mmol, 0.2 Eq), [1,1′-bis(diphenylphosphino)ferrocene]dichloropalladium(II) catalyst (44.26 mg, 0.0605 mmol, 0.1 Eq) and 1,1-dimethyl-prop-2-ynylamine (75.50 mg, 0.095 mL, 0.9082 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 20% MeOH in EtOAc) followed by HPLC purification to afford the titled compound as a solid (154.32 mg, 0.423 mmol, 70%). ¹H NMR (500 MHz, DMSO-d₆) δ 11.86 (s, 1H), 10.23 (s, 1H), 8.31-8.26 (m, 1H), 8.20-8.12 (m, 2H), 7.53 (d, J=1.6 Hz, 1H), 7.36 (dd, J=5.3, 1.7 Hz, 1H), 5.67 (s, 2H), 3.71 (s, 3H), 2.41 (s, 2H), 1.44 (s, 6H) ppm. LRMS (ESI+ve): For C₁₉H₂₀N₆O₂ requires 364.41 found 365.2 (M+H).

### Example 182

### Methyl (4-(3-amino-7-(3-methoxy-3-methylbut-1-yn-1-yl)-1H-indazol-5-yl)pyridin-2-yl)carbamate

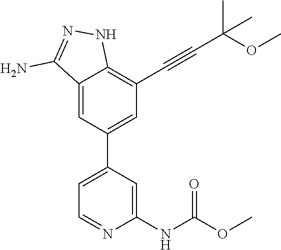 [SMILES: COC(=O)Nc1cc(-c2cc(C#CC(C)(C)OC)c3[nH]nc(N)c3c2)ccn1]

To a solution of methyl (4-(3-amino-7-bromo-1H-indazol-5-yl)pyridin-2-yl)carbamate (200 mg, 0.552 mmol, 1 Eq) in a degassed 4:1 solution of DMF/triethylamine (5 mL) were added copper(I) iodide (21 mg, 0.11 mmol, 0.2 Eq), [1,1′-bis(diphenylphosphino)ferrocene]dichloropalladium(II) catalyst (40.4 mg, 0.055 mmol, 0.1 Eq) and 3-methoxy-3-methylbut-1-yne (81.26 mg, 0.1 mL, 0.828 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a solid (115.19 mg, 0.303 mmol, 55%). ¹H NMR (500 MHz, DMSO-d₆) δ 11.92 (s, 1H), 10.21 (s, 1H), 8.29 (d, J=5.3 Hz, 1H), 8.21 (d, J=1.6 Hz, 1H), 8.14 (d, J=1.7 Hz, 1H), 7.63 (d, J=1.7 Hz, 1H), 7.37 (dd, J=5.3, 1.7 Hz, 1H), 5.68 (s, 2H), 3.71 (s, 3H), 3.37 (s, 3H), 1.56 (s, 6H) ppm. LRMS (ESI+ve): For C₂₀H₂₁N₅O₃ requires 379.42 found 380.2 (M+H).

### Example 183

### Methyl (4-(3-amino-7-(3-morpholinoprop-1-yn-1-yl)-1H-indazol-5-yl)pyridin-2-yl)carbamate

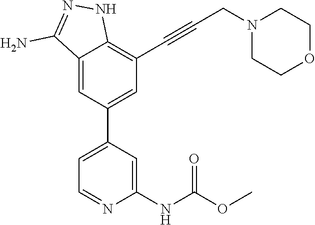 [SMILES: COC(=O)Nc1cc(-c2cc(C#CCN3CCOCC3)c3[nH]nc(N)c3c2)ccn1]

To a solution of methyl (4-(3-amino-7-bromo-1H-indazol-5-yl)pyridin-2-yl)carbamate (200 mg, 0.552 mmol, 1 Eq) in a degassed 4:1 solution of DMF/triethylamine (5 mL) were added copper(I) iodide (21 mg, 0.11 mmol, 0.2 Eq), [1,1′-bis(diphenylphosphino)ferrocene]dichloropalladium(II) catalyst (40.4 mg, 0.055 mmol, 0.1 Eq) and N-prop-2-ynyl-morpholine (103.63 mg, 0.103 mL, 0.828 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 20% MeOH in EtOAc) followed by HPLC purification to afford the titled compound as a solid (157.05 mg, 0.386 mmol, 70%). ¹H NMR (500 MHz, DMSO-d₆) δ 11.93 (s, 1H), 10.21 (s, 1H), 8.29 (d, J=5.3 Hz, 1H), 8.19 (d, J=1.6 Hz, 1H), 8.14 (d, J=1.7 Hz, 1H), 7.64 (d, J=1.7 Hz, 1H), 7.36 (dd, J=5.3, 1.7 Hz, 1H), 5.66 (s, 2H), 3.71 (s, 3H), 3.63 (t, J=4.7 Hz, 4H), 3.60 (s, 2H), 2.61-2.56 (t, J=4.8 Hz, 4H) ppm. LRMS (ESI+ve): For C₂₁H₂₂N₆O₃ requires 406.45 found 407.3 (M+H).

### Example 184

### Methyl (4-(3-amino-7-(4-morpholinobut-1-yn-1-yl)-1H-indazol-5-yl)pyridin-2-yl)carbamate

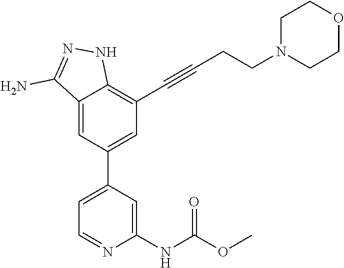 [SMILES: COC(=O)Nc1cc(-c2cc(C#CCCN3CCOCC3)c3[nH]nc(N)c3c2)ccn1]

To a solution of methyl (4-(3-amino-7-bromo-1H-indazol-5-yl)pyridin-2-yl)carbamate (200 mg, 0.552 mmol, 1 Eq) in a degassed 4:1 solution of DMF/triethylamine (5 mL) were added copper(I) iodide (21 mg, 0.11 mmol, 0.2 Eq), [1,1′-bis(diphenylphosphino)ferrocene]dichloropalladium(II) catalyst (40.4 mg, 0.055 mmol, 0.1 Eq) and 4-(but-3-yn-1-yl)morpholine (115.24 mg, 0.118 mL, 0.828 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a solid (116.05 mg, 0.276 mmol, 50%). ¹H NMR (500 MHz, DMSO-d₆) δ 11.88 (s, 1H), 10.21 (s, 1H), 8.28 (d, J=5.3 Hz, 1H), 8.20-8.09 (m, 2H), 7.57 (d, J=1.6 Hz, 1H), 7.35 (dd, J=5.2, 1.7 Hz, 1H), 5.64 (s, 2H), 3.71 (s, 3H), 3.60 (t, J=4.6 Hz, 4H), 2.69-2.65 (m, 4H), 2.47 (t, J=4.7 Hz, 4H) ppm.

LRMS (ESI+ve): For C₂₂H₂₄N₆O₃ requires 420.47 found 421.3 (M+H).

### Example 185

### 1-(4-(3-Amino-7-(3,3-dimethylbut-1-yn-1-yl)-1H-indazol-5-yl)pyridin-2-yl)urea

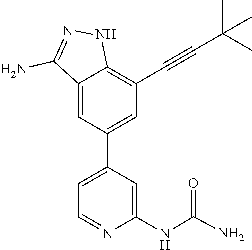 [SMILES: CC(C)(C)C#Cc1cc(-c2ccnc(NC(N)=O)c2)cc2c(N)n[nH]c12]

In a sealed 10 mL microwave vial methyl (4-(3-amino-7-(3,3-dimethylbut-1-yn-1-yl)-1H-indazol-5-yl)pyridin-2-yl)carbamate (Example 190) (30 mg, 0.082 mmol, 1 Eq) was dissolved in 2 mL of THF, then ammonia solution 0.5M in THF (55.85 mg, 0.066 mL, 3.28 mmol, 40 Eq) was added under argon atmosphere. The resulting reaction mixture was heated at 180° C. under microwave irradiation for 2 hours.

The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford a crude that was purified by HPLC to obtain the titled compound as a yellow solid (9.99 mg, 0.0287 mmol, 35%). ¹H NMR (500 MHz, DMSO-d₆) δ 11.85 (s, 1H), 9.11 (s, 1H), 8.22 (d, J=5.3 Hz, 1H), 8.14 (d, J=1.7 Hz, 1H), 7.74 (s, 1H), 7.50 (d, J=1.6 Hz, 1H), 7.24 (dd, J=5.5, 1.7 Hz, 1H), 6.97 (br. s. 2H), 5.64 (s, 2H), 1.38 (s, 9H) ppm. LRMS: For Cl₉H₂₀N₆O requires 348.41 found 349.3 (M+H).

### Example 186

### 1-(4-(3-Amino-7-(3,3-dimethylbut-1-yn-1-yl)-1H-indazol-5-yl)pyridin-2-yl)-3-methylurea

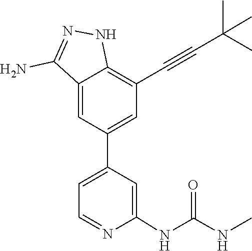 [SMILES: CNC(=O)Nc1cc(-c2cc(C#CC(C)(C)C)c3[nH]nc(N)c3c2)ccn1]

In a sealed 10 mL microwave vial methyl (4-(3-amino-7-(3,3-dimethylbut-1-yn-1-yl)-1H-indazol-5-yl)pyridin-2-yl)carbamate (Example 190) (30 mg, 0.082 mmol, 1 Eq) was dissolved in 2 mL of THF, then methylamine 2M in THF (76.91 mg, 0.090 mL, 2.47 mmol, 30 Eq) was added under argon atmosphere. The resulting reaction mixture was heated at 180° C. under microwave irradiation for 2 hours.

The reaction mixture was then cooled to ca. 40° C. and diluted by dropwise addition of water (5 mL) via syringe. The mixture was stirred and sonicated for 30 minutes and allowed to cool to room temperature. The cap was then removed and a solid was filtered, washed with water (2×4 mL) and dried to afford the title compound as a brown solid (20.21 mg, 0.055 mmol, 68%). ¹H NMR (500 MHz, DMSO-d₆) δ 11.84 (s, 1H), 9.19 (s, 1H), 8.20 (d, J=5.4 Hz, 1H), 8.11 (d, J=1.7 Hz, 1H), 8.10-8.02 (m, 1H), 7.65 (s, 1H), 7.49 (d, J=1.6 Hz, 1H), 7.22 (dd, J=5.4, 1.7 Hz, 1H), 5.62 (s, 2H), 2.75 (d, J=4.6 Hz, 3H), 1.37 (s, 9H) ppm. LRMS: For C₂₀H₂₂N₆O requires 362.44 found 363.1 (M+H).

### Example 187

### 1-(4-Iodopyridin-2-yl)-3-ethylurea

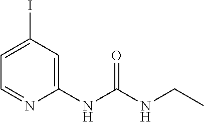

To a solution of 2-amino-4-iodopyridine (0.38 g, 1.73 mmol) in THF (2 mL) in a sealed microwave tube flushed with argon was added ethyl isocyanate (0.123 g, 0.136 mL, 1.73 mmol) under a blanket of argon and heated in a microwave reactor at 100 for 3 hours. The reaction was then quenched by pouring into water 10 mL and extracted with EtOAc (3×30 mL) and washed with brine (10 mL). The combined organic layers were concentrated to afford the crude product which was then purified by flash column chromatography eluting with a gradient of DCM:DCM:NH₃ MeOH 10% (0-50%) to afford the target compound as white solid (0.35 g, 69%). ¹H NMR (500 MHz, DMSO-d₆) δ 9.17 (s, 1H), 7.93 (d, J=1.5 Hz, 1H), 7.90 (d, J=5.3 Hz, 1H), 7.71 (s, 1H), 7.31 (dd, J=5.3, 1.5 Hz, 1H), 3.17 (qd, J=7.2, 5.4 Hz, 2H), 1.08 (t, J=7.2 Hz, 3H). LRMS (ESI+ve): For C₈H₁₀N₃O Molecular Weight: 291.0925 found 291.7

### 1-(4-(3-Amino-7-bromo-1H-indazol-5-yl)pyridin-2-yl)-3-ethylurea

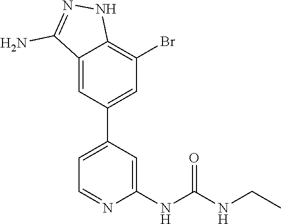 [SMILES: CCNC(=O)Nc1cc(-c2cc(Br)c3[nH]nc(N)c3c2)ccn1]

To a solution of 1-(4-iodopyridine-2-yl)-3-ethylurea (0.35 g, 1.2 mmol) in a degassed solution of dioxane:H₂O (18:2 5 mL) was added cesium carbonate (1.175 g, 3.607 mmol), 7-bromo-5-(4,4,5,5-tetramethyl-1,3,2-dioxaborolan-2-yl)-1H-indazol-3-amine (0.485 g, 1.44 mmol) and bis(triphenylphosphine)palladium(II) dichloride catalyst (0.0842 g, 0.12 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.12 g, 27%). ¹H NMR (500 MHz, DMSO-d₆) δ 12.01 (s, 1H), 9.14 (s, 1H), 8.23 (d, J=5.4 Hz, 1H), 8.17 (d, J=1.4 Hz, 1H), 8.03 (s, 1H), 7.75 (d, J=1.3 Hz, 1H), 7.71 (d, J=1.6 Hz, 1H), 7.23 (dd, J=5.5, 1.7 Hz, 1H), 5.70 (s, 2H), 3.21 (dtd, J=15.8, 8.6, 7.9, 5.4 Hz, 2H), 1.12 (t, J=7.2 Hz, 3H). LRMS (ESI+ve): For C₁₅H₁₅BrN₆O Molecular Weight: 375.2300 found 375.0, 376.0

### 1-(4-(3-Amino-7-(3,3-dimethylbut-1-yn-1-yl)-1H-indazol-5-yl)pyridin-2-yl)-3-ethylurea

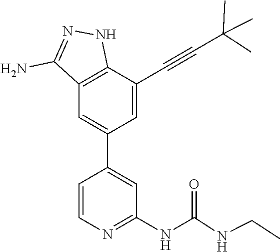 [SMILES: CCNC(=O)Nc1cc(-c2cc(C#CC(C)(C)C)c3[nH]nc(N)c3c2)ccn1]

To a solution of 1-(4-(3-amino-7-bromo-1H-indazol-5-yl)pyridin-2-yl)-3-ethylurea (0.12 g, 0.320 mmol) in a degassed 4:1 solution of DMF:triethylamine (5 mL) was added [1,1′-bis(diphenylphosphino)ferrocene]dichloropalladium(II) catalyst (0.0234 g, 0.032 mmol) copper iodide (0.012 g, 0.064 mmol) and 3,3-dimethylbut-1-yne (0.0315 g, 0.0473 mL, 0.384 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a yellow solid (0.028 g. 23%). ¹H NMR (500 MHz, DMSO-d₆) δ 11.85 (s, 1H), 9.11 (s, 1H), 8.21 (d, J=5.4 Hz, 1H), 8.13 (d, J=1.6 Hz, 1H), 8.08 (s, 1H), 7.70 (d, J=1.6 Hz, 1H), 7.50 (d, J=1.5 Hz, 1H), 7.23 (dd, J=5.5, 1.7 Hz, 1H), 5.63 (s, 2H), 3.22 (qd, J=7.1, 5.4 Hz, 2H), 1.38 (s, 9H) 1.12 (t, J=7.2 Hz, 3H). LRMS (ESI+ve): For C₂₁H₂₄N₆O Molecular Weight: 376.4640 found 377.3

### Example 188

### 1-(4-Iodopyridin-2-yl)-3-propylurea

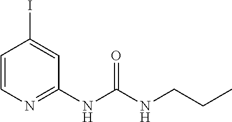

To a solution of 2-amino-4-iodopyridine (0.38 g, 1.73 mmol) in THF (2 mL) in a sealed microwave tube flushed with argon was added propyl isocyanate (0.147 g, 0.163 mL, 1.73 mmol) under a blanket of argon and heated in a microwave reactor at 100° C. for 3 hours. The reaction was then quenched by pouring into water 10 mL and extracted with EtOAc (3×30 mL) and washed with brine (10 mL). The combined organic layers were concentrated to afford the crude product which was then purified by flash column chromatography eluting with a gradient of DCM:DCM:NH₃ MeOH 10% (0-50%) to afford the target compound as white solid (0.37 g, 70%). ¹H NMR (500 MHz, DMSO-d₆) δ 9.17 (s, 1H), 7.93 (d, J=1.4 Hz, 1H), 7.90 (d, J=5.3 Hz, 1H), 7.76 (t, J=5.4 Hz, 1H), 7.31 (dd, J=5.3, 1.5 Hz, 1H), 3.11 (q, J=6.6 Hz, 2H), 1.47 (h, J=7.3 Hz, 2H), 0.89 (t, J=7.4 Hz, 3H). LRMS (ESI+ve): For Chemical Formula: C₉H₁₂IN₃O Molecular Weight: 305.1195 found 306.0

### 1-(4-(3-Amino-7-bromo-1H-indazol-5-yl)pyridin-2-yl)-3-propylurea

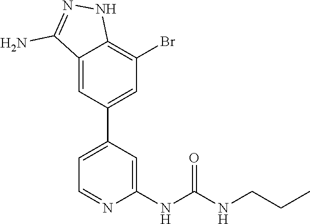 [SMILES: CCCNC(=O)Nc1cc(-c2cc(Br)c3[nH]nc(N)c3c2)ccn1]

To a solution of 1-(4-iodopyridine-2-yl)-3-propylurea(3 g, 1.21 mmol) in a degassed solution of dioxane:H₂O (18:2 5 mL) was added cesium carbonate (1.17 g, 3.6 mmol), 7-bromo-5-(4,4,5,5-tetramethyl-1,3,2-dioxaborolan-2-yl)-1H-indazol-3-amine (0.485 g, 1.44 mmol) and bis(triphenylphosphine)palladium(II) dichloride catalyst (0.085 g, 0.12 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.16 g, 34%). ¹H NMR (500 MHz, DMSO-d₆) δ 12.01 (s, 1H), 9.15 (s, 1H), 8.23 (d, J=5.4 Hz, 1H), 8.17 (d, J=1.4 Hz, 1H), 8.09 (s, 1H), 7.75 (d, J=1.4 Hz, 1H), 7.72 (d, J=1.6 Hz, 1H), 7.23 (dd, J=5.4, 1.7 Hz, 1H), 5.70 (s, 2H), 3.16 (q, J=6.6 Hz, 2H), 1.51 (h, J=7.2 Hz, 2H), 0.92 (t, J=7.4 Hz, 3H). LRMS (ESI+ve): For C₁₆H₁₇BrN₆O Molecular Weight: 389.2570 found 389.0, 390.0

### 1-(4-(3-Amino-7-(3,3-dimethylbut-1-yn-1-yl)-1H-indazol-5-yl)pyridin-2-yl)-3-propylurea

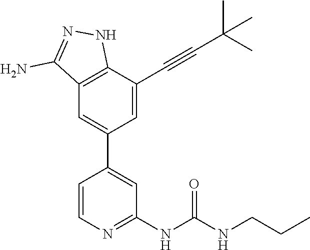 [SMILES: CCCNC(=O)Nc1cc(-c2cc(C#CC(C)(C)C)c3[nH]nc(N)c3c2)ccn1]

To a solution of 1-(4-(3-amino-7-bromo-1H-indazol-5-yl)pyridin-2-yl)-3-propylurea (0.16 g, 0.411 mmol) in a degassed 4:1 solution of DMF:triethylamine (5 mL) was added copper iodide (0.015 g, 0.082 mmol) [1,1′-bis(diphenylphosphino)ferrocene]dichloropalladium (II) catalyst (0.03 g, 0.0411 mmol) and 3,3-dimethylbut-1-yne (0.0405 g, 0.060 mL, 0.493 mmol), under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a yellow solid (0.036 g. 23%). ¹H NMR (500 MHz, DMSO-d₆) δ 11.84 (s, 1H), 9.12 (s, 1H), 8.21 (d, J=5.4 Hz, 1H), 8.13 (d, J=1.6 Hz, 2H), 7.71 (d, J=1.6 Hz, 1H), 7.50 (d, J=1.5 Hz, 1H), 7.23 (dd, J=5.4, 1.7 Hz, 1H), 5.63 (s, 2H), 3.16 (q, J=6.6 Hz, 2H), 1.51 (h, J=7.3 Hz, 2H), 1.38 (s, 9H) 0.92 (t, J=7.4 Hz, 3H). LRMS (ESI+ve): For C₂₂H₂₆N₆O Molecular Weight: 390.4910 found 391.3

### Example 189

### 1-(4-Iodopyridin-2-yl)-3-phenylurea

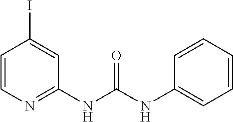 [SMILES: O=C(Nc1ccccc1)Nc1cc(I)ccn1]

To a solution of 2-amino-4-iodopyridine (0.38 g, 1.73 mmol) in THF (2 mL) in a sealed microwave tube flushed with argon was added phenyl isocyanate (0.206 g, 0.189 mL, 1.73 mmol) under a blanket of argon and heated in a microwave reactor at 100° C. for 3 hours. The reaction was then quenched by pouring into water 10 mL and extracted with EtOAc (3×30 mL) and washed with brine (10 mL). The combined organic layers were concentrated to afford the crude product which was then purified by flash column chromatography eluting with a gradient of DCM:DCM:NH₃ MeOH 10% (0-50%) to afford the target compound as a white solid (0.41 g, 70%). ¹H NMR (500 MHz, DMSO-d₆) δ 10.04 (s, 1H), 9.42 (s, 1H), 8.08 (d, J=1.4 Hz, 1H), 8.00 (d, J=5.3 Hz, 1H), 7.53-7.49 (m, 2H), 7.42 (dd, J=5.3, 1.5 Hz, 1H), 7.35-7.30 (m, 2H), 7.06-7.02 (m, 1H). LRMS (ESI+ve): For C₁₂H₁₀IN₃O Molecular Weight: 339.1365 found 339.7

### 1-(4-(3-Amino-7-bromo-1H-indazol-5-yl)pyridin-2-yl)-3-phenylurea

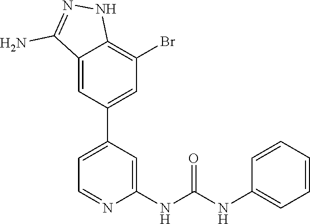 [SMILES: Nc1n[nH]c2c(Br)cc(-c3ccnc(NC(=O)Nc4ccccc4)c3)cc12]

To a solution of 1-(4-iodopyridine-2-yl)-3-phenylurea (0.41 g, 1.21 mmol) in a degassed solution of dioxane:H₂O (18:2 5 mL) was added cesium carbonate (1.182 g, 3.627 mmol), 7-bromo-5-(4,4,5,5-tetramethyl-1,3,2-dioxaborolan-2-yl)-1H-indazol-3-amine (0.488 g, 1.45 mmol) and bis(triphenylphosphine)palladium(II) dichloride catalyst (0.085 g, 0.121 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.17 g, 33%). ¹H NMR (500 MHz, DMSO-d₆) δ 12.03 (s, 1H), 10.40 (s, 1H), 9.43 (s, 1H), 8.33 (d, J=5.5 Hz, 1H), 8.22 (d, J=1.5 Hz, 1H), 7.87 (d, J=1.6 Hz, 1H), 7.80 (d, J=1.5 Hz, 1H), 7.57-7.54 (m, 2H), 7.36-7.31 (m, 4H), 7.05 (d, J=7.4 Hz, 1H), 5.73 (s, 2H). LRMS (ESI+ve): For Chemical Formula: C₁₉H₁₅BrN₆O Molecular Weight: 423.2740 found 423.0, 424.0

### 1-(4-(3-Amino-7-(3,3-dimethylbut-1-yn-1-yl)-1H-indazol-5-yl)pyridine-2-yl)-3-phenylurea

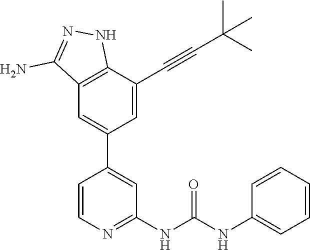 [SMILES: CC(C)(C)C#Cc1cc(-c2ccnc(NC(=O)Nc3ccccc3)c2)cc2c(N)n[nH]c12]

To a solution of 1-(4-(3-amino-7-bromo-1H-indazol-5-yl)pyridin-2-yl)-3-phenylurea (0.17 g, 0.402 mmol) in a degassed 4:1 solution of DMF:triethylamine (5 mL) was added [1,1′-bis(diphenylphosphino)ferrocene]dichloropalladium(II) catalyst (0.029 g, 0.0402 mmol), copper Iodide (0.0153 g, 0.0803 mmol) and 3,3-dimethylbut-1-yne (0.0396 g, 0.0594 mL 0.482 mmol), under an atmosphere of argon. The reaction was then sealed and heated at 90° C. for 18 hours.

The cooled reaction was diluted with EtOAc and washed with water (20 mL) and brine (10 mL).

The organic layer was concentrated under reduced pressure and purified by flash column chromatography utilising NH silica cartridges from Biotage eluting with 50-100% petroleum ether: EtOAc to afford the target compound as a an off-white powder (0.043 g. 25%). ¹H NMR (500 MHz, DMSO-d₆) δ 11.87 (s, 1H), 10.48 (s, 1H), 9.42 (s, 1H), 8.32 (d, J=5.4 Hz, 1H), 8.17 (d, J=1.6 Hz, 1H), 7.87-7.83 (m, 1H), 7.58-7.53 (m, 3H), 7.36-7.31 (m, 3H), 7.04 (tt, J=7.3, 1.2 Hz, 1H), 5.66 (s, 2H), 1.38 (s, 9H). LRMS (ESI+ve): For C₂₅H₂₄N₆O Molecular Weight: 424.5080 found 425.3.

### Example 190

### Methyl (4-(3-amino-7-(3,3-dimethylbut-1-yn-1-yl)-1H-indazol-5-yl)pyridin-2-yl)carbamate

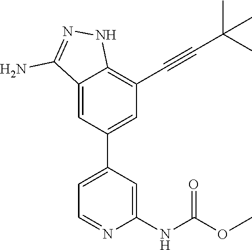 [SMILES: COC(=O)Nc1cc(-c2cc(C#CC(C)(C)C)c3[nH]nc(N)c3c2)ccn1]

To a solution of methyl (4-(3-amino-7-bromo-1H-indazol-5-yl)pyridin-2-yl)carbamate (200 mg, 0.552 mmol, 1 Eq) in a degassed 4:1 solution of DMF/triethylamine (2 mL) was added copper(I) iodide (21 mg, 0.11 mmol, 0.2 Eq), [1,1′-bis(diphenylphosphino)ferrocene]dichloropalladium(II) catalyst (40.4 mg, 0.055 mmol, 0.1 Eq) and 3,3-dimethylbut-1-yne (68.03 mg, 0.102 mL, 0.828 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (95% EtOAc in petroleum ether) followed by HPLC purification to give the titled product as white solid (122.4 mg, 0.33 mmol, 61%). ¹H NMR (500 MHz, DMSO-d₆) δ 11.83 (s, 1H), 10.21 (s, 1H), 8.28 (d, J=5.3 Hz, 1H), 8.14 (dd, J=9.8, 1.8 Hz, 2H), 7.52 (s, 1H), 7.36 (dd, J=5.3, 1.7 Hz, 1H), 5.64 (s, 2H), 3.71 (s, 3H), 1.37 (s, 9H) ppm. LRMS (ESI+ve): For C₂₀H₂₁N₅O₂ Molecular Weight: 363.42 found 364.0 (M+H).

### Example 191

### Ethyl (4-iodopyridin-2-yl)carbamate

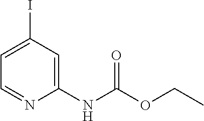

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise ethyl chloroformate (295.60 mg, 0.260 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (20% EtOAc in petroleum ether) to give the titled product as a white solid (380 mg, 1.30 mmol, 80%). ¹H NMR (500 MHz, DMSO-d₆) δ 10.25 (s, 1H), 8.26 (d, J=1.1 Hz, 1H), 7.98 (d, J=5.2 Hz, 1H), 7.44 (dd, J=5.2, 1.5 Hz, 1H), 4.14 (q, J=7.1 Hz, 2H), 1.24 (t, J=7.1 Hz, 3H) ppm.

### Ethyl (4-(3-amino-7-bromo-1H-indazol-5-yl)pyridin-2-yl)carbamate

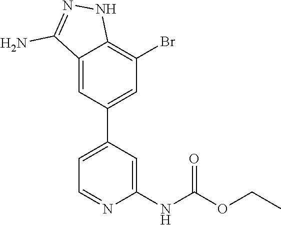 [SMILES: CCOC(=O)Nc1cc(-c2cc(Br)c3[nH]nc(N)c3c2)ccn1]

To a solution of 7-bromo-5-(4,4,5,5-tetramethyl-1,3,2-dioxaborolan-2-yl)-1H-indazol-3-amine (508.14 mg, 1.50 mmol, 1.1 Eq) in a degassed 9:1 solution of dioxane/water (5 mL), were added ethyl (4-iodopyridin-2-yl)carbamate (380 mg, 1.36 mmol, 1 Eq), cesium carbonate (1.33 g, 4.08 mmol, 3 Eq) and bis(triphenylphosphine)palladium chloride (95.46 mg, 0.136 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (90% EtOAc in petroleum ether), to afford the titled compound as a green solid (404 mg, 1.115 mmol, 82%). ¹H NMR (500 MHz, DMSO-d₆) δ 12.00 (s, 1H), 10.23 (s, 1H), 8.30 (d, J=5.3 Hz, 1H), 8.20 (d, J=1.5 Hz, 1H), 8.14 (d, J=1.7 Hz, 1H), 7.79 (d, J=1.5 Hz, 1H), 7.36 (dd, J=5.3, 1.7 Hz, 1H), 5.72 (s, 2H), 3.71 (s, 3H) ppm.

### Ethyl (4-(3-amino-7-(3,3-dimethylbut-1-yn-1-yl)-1H-indazol-5-yl)pyridin-2-yl)carbamate

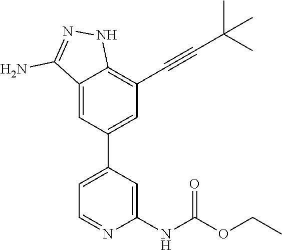 [SMILES: CCOC(=O)Nc1cc(-c2cc(C#CC(C)(C)C)c3[nH]nc(N)c3c2)ccn1]

To a solution of ethyl (4-(3-amino-7-bromo-1H-indazol-5-yl)pyridin-2-yl)carbamate (200 mg, 0.532 mmol, 1 Eq) in a degassed 4:1 solution of DMF/triethylamine (2 mL) were added copper(I) iodide (20.26 mg, 0.106 mmol, 0.2 Eq), [1,1′-bis(diphenylphosphino)ferrocene]dichloropalladium(II) catalyst (38.93 mg, 0.053 mmol, 0.1 Eq) and 3,3-dimethylbut-1-yne (65.55 mg, 0.098 mL, 0.798 mmol, 1.5 Eq), under an atmosphere of argon. The reaction was then sealed and heated at 70° C. for 18 hours. The cooled reaction was diluted with EtOAc (20 mL) and washed with water (10 mL) and brine (2×10 mL). The organic layer was concentrated under reduced pressure and the crude was purified by flash column chromatography (95% EtOAc in petroleum ether) followed by HPLC purification to give the titled product as a yellow solid (120.5 mg, 0.32 mmol, 60%). ¹H NMR (500 MHz, DMSO-d₆) 5 11.83 (br. s, 1H), 10.12 (s, 1H), 8.27 (d, J=5.3 Hz, 1H), 8.14 (dd, J=10.0, 1.7 Hz, 2H), 7.52 (d, J=1.7 Hz, 1H), 7.35 (dd, J=5.3, 1.7 Hz, 1H), 5.64 (s, 2H), 4.17 (q, J=7.1 Hz, 2H), 1.27 (t, J=7.1 Hz, 3H) ppm. LRMS (ESI+ve): For C₂₁H₂₃N₅O₂ Molecular Weight: 377.45 found 378.1 (M+H).

### Example 192

### tert-Butyl (4-(3-amino-7-bromo-1H-indazol-5-yl)pyridin-2-yl)carbamate

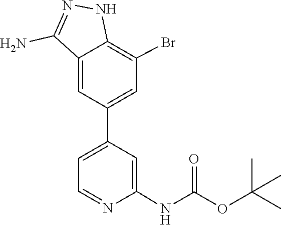 [SMILES: CC(C)(C)OC(=O)Nc1cc(-c2cc(Br)c3[nH]nc(N)c3c2)ccn1]

To a solution of tert-butyl (4-iodopyridin-2-yl)carbamate (0.344 g, 1.081 mmol) in a degassed solution of dioxane:H₂O (18:2 5 mL) was added caesium carbonate (1.056 g, 3.24 mmol), 7-bromo-5-(4,4,5,5-tetramethyl-1,3,2-dioxaborolan-2-yl)-1H-indazol-3-amine (0.437 g, 1.297 mmol) and bis(triphenylphosphine)palladium(II) dichloride catalyst (0.038 g, 0.054 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a brown solid (0.214 g, 49%). ¹H NMR (500 MHz, DMSO-d₆) δ 11.99 (s, 1H), 9.79 (s, 1H), 8.28 (d, J=5.3 Hz, 1H), 8.19 (d, J=1.4 Hz, 1H), 8.09 (d, J=1.5 Hz, 1H), 7.79 (d, J=1.5 Hz, 1H), 7.33 (dd, J=5.3, 1.7 Hz, 1H), 5.72 (s, 2H), 1.51 (s, 9H). LRMS (ESI+ve): For C₁₇H₁₈BrN₅O₂ Molecular Weight: 404.2680 found 404.0, 405.0

### tert-Butyl (4-(3-amino-7-(3,3-dimethylbut-1-yn-1-yl)-1H-indazol-5-yl)pyridin-2-yl)carbamate

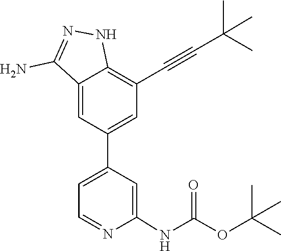 [SMILES: CC(C)(C)C#Cc1cc(-c2ccnc(NC(=O)OC(C)(C)C)c2)cc2c(N)n[nH]c12]

To a solution of tert-Butyl (4-(3-amino-7-bromo-1H-indazol-5-yl)pyridin-2-yl)carbamate (0.25 g, 0.62 mmol) in a degassed 4:1 solution of DMF:triethylamine (5 mL) was added [1,1′-bis(diphenylphosphino)ferrocene]dichloropalladium(II) catalyst (0.045 g, 0.062 mmol) and copper iodide (0.0236 g, 0.124 mmol), 3,3-dimethylbut-1-yne (0.061 g, 0.092 mL, 0.744 mmol), under an atmosphere of argon. The reaction was then sealed and heated at 90° C. for 18 hours.

The cooled reaction was diluted with EtOAc and washed with water (20 mL) and brine (10 mL).

The organic layer was concentrated under reduced pressure and purified by flash column chromatography utilising NH silica cartridges from Biotage eluting with 50-100% petroleum ether: EtOAc to afford the target compound as a brown solid (0.186 g, 35%). ¹H NMR (500 MHz, DMSO-d₆) δ 11.83 (s, 1H), 9.77 (s, 1H), 8.27 (d, J=5.3 Hz, 1H), 8.14 (d, J=1.6 Hz, 1H), 8.08 (d, J=1.6 Hz, 1H), 7.51 (d, J=1.7 Hz, 1H), 7.32 (dd, J=5.2, 1.7 Hz, 1H), 5.65 (s, 2H), 1.51 (s, 9H), 1.38 (s, 9H). LRMS (ESI+ve): For Chemical Formula: C₂₃H₂₇N₅O₂ Molecular Weight: 405.5020 found 406.3

### Example 193

### N-(4-Iodopyridin-2-yl)methanesulfonamide

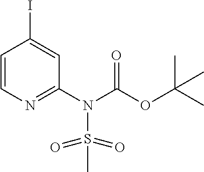 [SMILES: CC(C)(C)OC(=O)N(c1cc(I)ccn1)S(C)(=O)=O]

To a solution of tert-butyl (4-iodopyridin-2-yl)carbamate (0.5 g, 1.56 mmol) in dioxane (2 mL) was added NaHMDS (1.2 eq, 1M solution in THF) at 0° C. under a blanket of argon followed by methanesulfonyl chloride (0.57 g, 5 mmol) and the resulting reaction mixture allowed to warm up to room temperature and stirred at this temperature for 12 hours. The reaction was then quenched by pouring over ice/water 20 mL and extracted with EtOAc (3×30 mL) and washed with brine (10 mL). The combined organic layers were concentrated to afford the crude product which was taken up in DCM (5 mL) and stirred at 0° C. with 2 ml TFA (1M_{aq}) for 3 hours, then quenched by pouring over ice/saturated NaHCO_{3Aq} 10 mL and extracted with EtOAc (3×30 mL), washed with brine (10 mL), concentrated and purified by flash column chromatography eluting with a gradient of EtOAc:Petroleum Ether (0-50%) to afford the target compound as a light yellow coloured solid (0.34 g, 73%). ¹H NMR (500 MHz, DMSO-d₆) δ 8.24 (d, J=5.2 Hz, 1H), 8.00 (d, J=1.4 Hz, 1H), 7.91 (dd, J=5.1, 1.5 Hz, 1H), 3.52 (s, 3H), 1.40 (s, 9H). LRMS (ESI+ve): For C₁₁H₁₅IN₂O₄S Molecular weight 398.2155 found 399.0

### tert-Butyl (4-(3-amino-7-bromo-1H-indazol-5-yl)pyridin-2-yl)(methylsulfonyl)carbamate

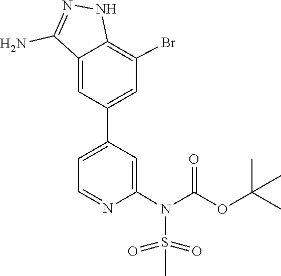 [SMILES: CC(C)(C)OC(=O)N(c1cc(-c2cc(Br)c3[nH]nc(N)c3c2)ccn1)S(C)(=O)=O]

To a solution of tert-butyl (4-iodopyridin-2-yl)(methylsulfonyl)carbamate (0.11 g, 0.28 mmol) in a degassed solution of dioxane:H₂O (18:2 5 mL) was added caesium carbonate (0.274 g, 0.84 mmol), 7-bromo-5-(4,4,5,5-tetramethyl-1,3,2-dioxaborolan-2-yl)-1H-indazol-3-amine (0.113 g, 0.34 mmol) and bis(triphenylphosphine)palladium(II) dichloride catalyst (0.020 g, 0.028 mmol) under an atmosphere of argon. The reaction was then sealed and stirred at 90° C. for 18 hours.

The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a dark brown oil (0.089 g, 65%). ¹H NMR (500 MHz, DMSO-d₆) δ 12.09 (s, 1H), 8.57 (d, J=5.2 Hz, 1H), 8.42 (s, 1H), 8.04 (d, J=1.5 Hz, 1H), 8.01-7.92 (m, 2H), 5.73 (s, 2H), 3.58 (s, 3H), 1.43 (s, 9H). LRMS (ESI+ve): For C₁₈H₂₀BrN₅O₄S Molecular weight 482.3530 found 482.1

### N-(4-(3-Amino-7-(3,3-dimethylbut-1-yn-1-yl)-1H-indazol-5-yl)pyridine-2-yl)methanesulfonamide

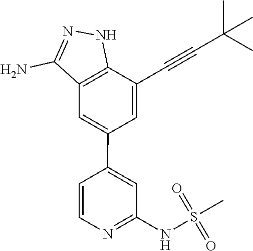 [SMILES: CC(C)(C)C#Cc1cc(-c2ccnc(NS(C)(=O)=O)c2)cc2c(N)n[nH]c12]

To a solution of tert-butyl (4-(3-amino-7-bromo-1H-indazol-5-yl)pyridin-2-yl)(methylsulfonyl)carbamate (0.089 g, 0.184 mmol) in a degassed 4:1 solution of DMF:triethylamine (5 mL) was added [1,1′-bis(diphenylphosphino)ferrocene]dichloropalladium(II) catalyst (0.010 g, 0.013 mmol), copper iodide (0.005 g, 0.026 mmol) and 3,3-dimethylbut-1-yne (0.018 g, 0.027 mL 0.219 mmol), under an atmosphere of argon. The reaction was then sealed and heated at 90° C. for 18 hours.

The cooled reaction was diluted with EtOAc and washed with water (20 mL) and brine (10 mL).

The organic layer was concentrated under reduced pressure and treated with TFA/DCM (1%) at 0° C. for 1 hour quenched with saturated sodium bicarbonate (10 mL) and the organic layer concentrated under reduced pressure followed by purification by HPLC to afford the target compound as a yellow solid (0.023 g. 33%). ¹H NMR (500 MHz, DMSO-d₆) δ 11.11 (s, 1H), 8.48-8.46 (m, 1H), 8.44 (d, J=5.4 Hz, 1H), 8.32-8.28 (m, 2H), 7.94 (dd, J=5.0, 1.1 Hz, 1H), 7.69 (d, J=1.7 Hz, 1H), 7.56 (dd, J=5.4, 1.8 Hz, 1H), 7.26 (dd, J=5.0, 3.8 Hz, 1H), 1.39 (s, 9H). LRMS (ESI+ve): For C₁₉H₂₁N₅O₂S Molecular Weight: 383.4700 found 386.2.

### Example 194

### N-(4-iodopyridin-2-yl)methanesulfonamide

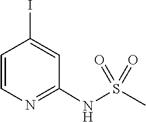

To a solution of 4-iodopyridin-2-amine (380 mg, 1.36 mmol) in 3.4 ml of pyridine cold down at 0° C. was added methyl sulfonyl chloride (0.12 mL, 1.52 mmol). The reaction mixture was run at RT for 2 hours. The resulting mixture was diluted with EtOAc, quenched with NaHCO₃, extracted with EtOAc, dry over MgSO₄, concentrated under reduced pressure and the residue was purified by column chromatography (First column: Toluene/EtOAc 7:3; Second column Petrol 60-80/Acetone 8:2) to give the product as an orange solid. (120 mg, 30%). ¹H NMR (400 MHz, DMSO-d₆ and (CD₃)₂CO): δ 3.14 (s, 3H), 7.41 (dd, J=5.4, 1.5 Hz, 1H), 7.45 (d, J=1.5 Hz, 1H), 7.98 (d, J=5.4 Hz, 1H), 10.74 (br s, 1H). LRMS: Calculated for C₁₉H₂₆BN₃O₂ 297.9 found 298.9 (M+1)

### N-(4-(3-amino-7-(3,3-dimethylbut-1-yn-1-yl)-1H-indazol-5-yl)pyridin-2-yl)methanesulfonamide

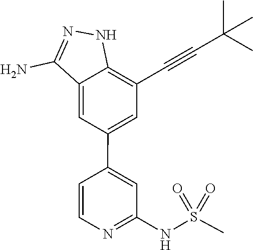 [SMILES: CC(C)(C)C#Cc1cc(-c2ccnc(NS(C)(=O)=O)c2)cc2c(N)n[nH]c12]

A solution of N-(4-iodopyridin-2-yl)methanesulfonamide (60 mg, 0.20 mmol), Cs₂CO₃ (217 mg, 0.67 mmol), bis(triphenylphosphine) palladium(II) chloride (38 mg, 0.05 mmol) and 7-(3,3-dimethylbut-1-yn-1-yl)-5-(4,4,5,5-tetramethyl-1,3,2-dioxaborolan-2-yl)-1H-indazol-3-amine (76 mg, 0.22 mmol) in 1 ml of dioxane-water (4:1) was degassed in sealed tube and submitted to reaction. The reaction mixture was heated to 80° C., o/n. The resulting mixture was diluted with EtOAc, filtered through silica, concentrated under reduced pressure and the residue was purified by HPLC to afford the title compound as a dark yellow solid. (12 mg, 16%). ¹H NMR (400 MHz, (CD₃)₂CO): δ 1.38 (s, 9H), 3.30 (s, 3H), 5.13 (br s, 2H), 7.36 (dd, J=5.5, 1.7 Hz, 1H), 7.44 (dd, J=1.7, 0.8 Hz, 1H), 7.65 (d, J=1.6 Hz, 1H), 8.14 (d, J=1.6 Hz, 1H), 8.29 (dd, J=5.5, 0.8 Hz, 1H), 11.15 (br s, 1H), 1 proton missing. LRMS: Calculated for C₁₉H₂₁N₅O₂S 383.1 found 384.1 (M+1).

### Example 195

### 6-Fluoro-4-iodopyridin-2-amine

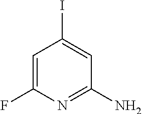

To a solution of 2,6-difluoro-4-iodopyridine (1.15 g, 4.77 mmol) in dioxane (5 mL) was added ammonium hydroxide (10 mL 28% in H₂O) the reaction was the flushed with argon, sealed and stirred at 80° C. for 3 hours by microwave irradiation. The reaction was cooled, partitioned between EtOAc and H₂O and the aqueous layer washed a further 2 times with EtOAc (2×30 mL). The combined organic layers were washed with brine and concentrated under reduced pressure. Purification by flash column chromatography afforded the desired compound as a clear oil (0.90 g, 79%). ¹H NMR (400 MHz, DMSO-d₆) δ 7.61 (d, J=5.4 Hz, 1H), 6.87 (d, J=1.5 Hz, 1H), 6.82 (dd, J=5.3, 1.5 Hz, 1H), 6.08 (s, 2H). ¹⁹F NMR (471 MHz, DMSO-d₆) δ −70.62. LRMS (ESI+ve): For C₅H₄IFN₂ Molecular weight 238.0039 found 239.0

### N-(6-Fluoro-4-iodopyridin-2-yl)acetamide

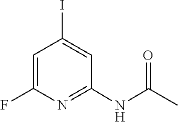

To a solution of 6-fluoro-4-iodopyridin-2-amine (0.3 g, 0.3 mmol) in dioxane cooled to 0° C. was added NaH (0.181 g, 4.54 mmol, 60% suspension, 2 eq) under a blanket of argon. Acetyl chloride (0.18 g, 0.16 mmol) was then added dropwise and the resulting reaction mixture allowed to warm up to room temperature and stirred at this temperature for 3 hours. The reaction was then quenched by pouring over ice/water 20 mL, extracted with EtOAc (3×30 mL) and washed with brine (10 mL). The combined organic layers were concentrated and purified by flash eluting with a gradient of EtOAc:Petroleum Ether (50-100%) to afford the target compound as a cream coloured solid (0.21 g, 60%). ¹H NMR (400 MHz, DMSO-d₆) δ 10.77 (s, 1H), 8.41 (t, J=1.3 Hz, 1H), 7.34 (dd, J=2.8, 1.0 Hz, 1H), 2.09 (s, 3H). ¹⁹F NMR (376 MHz, DMSO-d₆) δ −69.67.

### N-(4-(3-Amino-7-bromo-1H-indazol-5-yl)-6-fluoropyridin-2-yl)acetamide

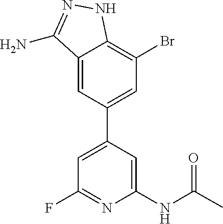 [SMILES: CC(=O)Nc1cc(-c2cc(Br)c3[nH]nc(N)c3c2)cc(F)n1]

To a solution of N-(6-fluoro-4-iodopyridin-2-yl)acetamide (0.21 g, 0.75 mmol) in a degassed solution of dioxane:H₂O (18:2 5 mL) was added cesium carbonate (0.733 g, 2.25 mmol), 7-bromo-5-(4,4,5,5-tetramethyl-1,3,2-dioxaborolan-2-yl)-1H-indazol-3-amine (0.303 g, 0.90 mmol) and bis(triphenylphosphine)palladium(II) dichloride catalyst (0.053 g, 0.075 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a yellow solid (0.177 g, 65%). ¹H NMR (500 MHz, DMSO-d₆) δ 12.06 (s, 1H), 10.71 (s, 1H), 8.33 (d, J=1.7 Hz, 1H), 8.25 (d, J=1.5 Hz, 1H), 7.85 (d, J=1.5 Hz, 1H), 7.17 (s, 1H), 5.75 (s, 2H), 2.14 (s, 3H). ¹⁹F NMR (471 MHz, DMSO-d₆) δ −70.62. LRMS (ESI+ve): For C₁₄H₁₁BrFN₅O Molecular Weight: 364.1784 found 364.0, 365.0

### N-(4-(3-amino-7-(3,3-dimethylbut-1-yn-1-yl)-1H-indazol-5-yl)-6-fluoropyridin-2-yl)acetamide

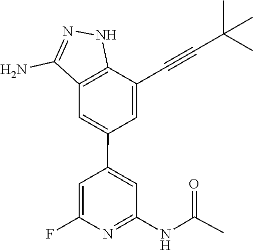 [SMILES: CC(=O)Nc1cc(-c2cc(C#CC(C)(C)C)c3[nH]nc(N)c3c2)cc(F)n1]

To a solution of N-(4-(3-amino-7-bromo-1H-indazol-5-yl)-6-fluoropyridin-2-yl)acetamide (0.35 g, 0.961 mmol) in a degassed 4:1 solution of DMF:triethylamine (5 mL) was added [1,1′-bis(diphenylphosphino)ferrocene]dichloropalladium(II) catalyst (0.035 g, 0.048 mmol) copper iodide (0.018 g, 0.096 mmol) and 3,3-dimethylbut-1-yne (0.082 g, 1.0 mmol) under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatographyutilising NH silica cartridges from Biotage eluting with 50-100% petroleum ether: EtOAc to afford the target compound as a cream colored solid (0.112 g. 32%). ¹H NMR (500 MHz, DMSO-d₆) δ 11.91 (s, 1H), 10.70 (s, 1H), 8.32 (s, 1H), 8.21 (s, 1H), 7.57 (s, 1H), 7.15 (s, 1H), 5.68 (s, 2H), 2.14 (s, 3H), 1.38 (s, 9H). ¹⁹F NMR (471 MHz, DMSO-d₆) δ −70.80. LRMS (ESI+ve): For C₂₀H₂₀FN₅O Molecular Weight: 365.4124 found 366.3

### Examples 196 and 197

### N-(4-Iodopyridin-2-yl)acetamide

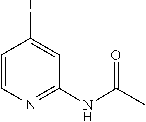

To a solution of 4-iodopyridin-2-amine (0.5 g 2.27 mmol) in dioxane cooled to 0° C. was added NaH (0.181 g, 4.54 mmol, 60% suspension, 2 eq) under a blanket of argon. Acetyl Chloride (0.18 g, 0.16 mmol) was then added dropwise and the resulting reaction mixture allowed to warm up to room temperature and stirred at this temperature for 3 hours. The reaction was then quenched by pouring over ice/water 20 mL, extracted with EtOAc (3×30 mL) and washed with brine (10 mL). The combined organic layers were concentrated and purified by flash eluting with a gradient of EtOAc:Petroleum Ether (50-100%) to afford the target compound as an off white solid (0.40 g, 67%)¹H NMR (400 MHz, DMSO-d₆) δ 10.59 (s, 1H), 8.52 (d, J=1.7 Hz, 1H), 8.03 (dd, J=5.2, 0.6 Hz, 1H), 7.50 (dd, J=5.2, 1.5 Hz, 1H), 2.09 (s, 3H). LRMS (ESI+ve): For C₇H₇IN₂O Molecular Weight: 262.0505 found 263.4

### N-(4-(3-Amino-7-bromo-1H-indazol-5-yl)pyridin-2-yl)acetamide

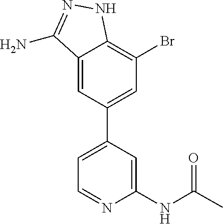 [SMILES: CC(=O)Nc1cc(-c2cc(Br)c3[nH]nc(N)c3c2)ccn1]

To a solution of N-(4-iodopyridin-2-yl)acetamide (0.2 g, 0.755 mmol) in a degassed solution of dioxane:H₂O (18:2 5 mL) was added cesium carbonate (0.738 g, 2.264 mmol), 7-bromo-5-(4,4,5,5-tetramethyl-1,3,2-dioxaborolan-2-yl)-1H-indazol-3-amine (0.31 g, 0.916 mmol) and bis(triphenylphosphine)palladium(II) dichloride catalyst (0.027 g, 0.038 mmol) under an atmosphere of argon. The reaction was then sealed and stirred at 90° C. for 18 hours. The cooled reaction was then diluted with EtOAc (30 mL) and washed with water (30 mL) and brine (10 mL). The organic layer was then concentrated under reduced pressure and purified by flash column chromatography eluting from 50-100% petroleum ether: EtOAc to afford the desired compound as a light brown solid (0.18 g, 68%). ¹H NMR (500 MHz, DMSO-d₆) δ 12.01 (s, 1H), 10.54 (s, 1H), 8.40 (s, 1H), 8.35 (d, J=5.3 Hz, 1H), 8.20 (d, J=1.4 Hz, 1H), 7.79 (s, 1H), 7.40 (dd, J=5.4, 1.7 Hz, 1H), 5.72 (s, 2H), 2.14 (s, 3H). LRMS (ESI+ve): C₁₄H₁₂BrN₅O Molecular Weight: 346.1880 found 346.0, 347.0

### N-(4-(3-Amino-7-phenyl-1H-indazol-5-yl)pyridin-2-yl)acetamide (Example 196)

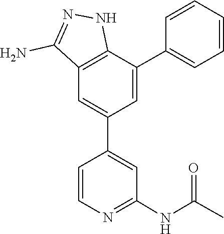 [SMILES: CC(=O)Nc1cc(-c2cc(-c3ccccc3)c3[nH]nc(N)c3c2)ccn1]

To a solution of N-(4-(3-amino-7-bromo-1H-indazol-5-yl)pyridin-2-yl)acetamide (0.07 g, 0.02022 mmol) in a degassed dioxane:H₂O (18:2 5 mL) was added cesium carbonate (0.1976 g, 0.6066 mmol), phenylboronic acid (0.1219 g, 0.02426 mmol) and [1,1′-bis(diphenylphosphino)ferrocene]dichloropalladium(II) catalyst (0.002 g, 0.002022 mmol) under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc to afford the target compound as a dark brown solid (0.058 g. 84%). ¹H NMR (500 MHz, DMSO-d₆) δ 11.74 (s, 1H), 10.52 (s, 1H), 8.48 (s, 1H), 8.36 (s, 1H), 8.19 (s, 1H), 7.83-7.71 (m, 2H), 7.64-7.45 (m, 5H), 5.63 (s, 2H), 2.14 (s, 3H). LRMS (ESI) m/z [M]⁺. For C₂₀H₁₇N₅O Molecular Weight: 343.3900 found 344.3

### N-(4-(3-Amino-7-(pyridin-4-yl)-1H-indazol-5-yl)pyridin-2-yl)acetamide (Example 197)

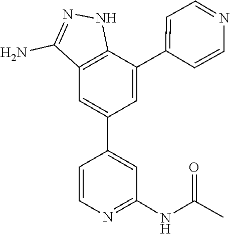 [SMILES: CC(=O)Nc1cc(-c2cc(-c3ccncc3)c3[nH]nc(N)c3c2)ccn1]

To a solution of N-(4-(3-amino-7-bromo-1H-indazol-5-yl)pyridin-2-yl)acetamide (0.07 g, 0.02022 mmol) in a degassed dioxane:H₂O (18:2 5 mL) was added cesium carbonate (0.1976 g, 0.6066 mmol), 4-pyridylboronic acid (0.1219 g, 0.02426 mmol) and [1,1′-bis(diphenylphosphino)ferrocene]dichloropalladium(II) catalyst (0.002 g, 0.002022 mmol) under an atmosphere of argon. The reaction was then sealed and heated at 90° C. for 18 hours. The cooled reaction was diluted with EtOAc and washed with water (20 mL) and brine (10 mL). The organic layer was concentrated under reduced pressure and purified by flash column chromatography utilising NH silica cartridges from Biotage eluting with 50-100% petroleum ether: EtOAc to afford the target compound as a yellow solid (0.034 g. 34%). ¹H NMR (500 MHz, DMSO-d₆) δ 12.08-11.75 (m, 1H), 10.55 (s, 1H), 8.72 (s, 2H), 8.55-8.25 (m, 3H), 7.80 (s, 3H), 7.52 (s, 1H), 5.74 (s, 2H), 2.15 (s, 3H). LRMS (ESI+ve): Chemical Formula: C₁₉H₁₉N₆O Molecular Weight: 344.3780 found 345.2

### Example 306

### Methyl (4-(3-amino-7-phenyl-1H-indazol-5-yl)pyridin-2-yl)carbamate SU1742

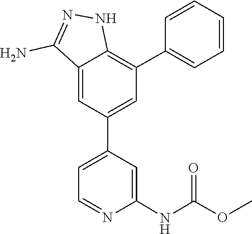 [SMILES: COC(=O)Nc1cc(-c2cc(-c3ccccc3)c3[nH]nc(N)c3c2)ccn1]

A solution of methyl (4-(3-amino-7-bromo-1H-indazol-5-yl)pyridin-2-yl)carbamate (1.1 g, 2.96 mmol, 1 Eq), phenylboronic acid (0.5 g, 4.44 mmol, 1.5 Eq), cesium carbonate (2.9 g, 8.88 mmol, 3 Eq) and [1,1′-bis(diphenylphosphino)ferrocene]dichloropalladium(II) (322 mg, 0.44 mmol, 0.15 Eq) in dioxane (8 mL) and water (2 mL) was stirred under nitrogen atmosphere at 80° C. for 1 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) to give the titled product as white solid (0.5 g, 1.4 mmol, 47%). 1H NMR (500 MHz, DMSO) δ 11.75 (s, 1H), 10.22 (s, 1H), 8.32 (d, J=5.3 Hz, 1H), 8.23 (d, J=1.6 Hz, 1H), 8.20 (d, J=1.7 Hz, 1H), 7.77 (d, J=7.6 Hz, 2H), 7.63 (d, J=1.6 Hz, 1H), 7.55 (t, J=7.6 Hz, 2H), 7.48-7.42 (m, 2H), 5.63 (s, 2H), 3.72 (s, 3H). LC-MS: For C₂₀H₁₇N₅O₂ requires 359.39 found 360.3 (M+H).

### Example 307

### Methyl (4-(3-amino-7-(4-(difluoromethyl)phenyl)-1H-indazol-5-yl)pyridin-2-yl)carbamate

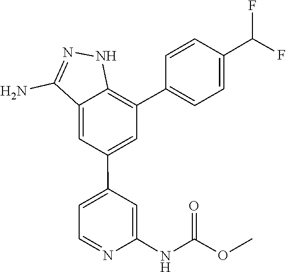 [SMILES: COC(=O)Nc1cc(-c2cc(-c3ccc(C(F)F)cc3)c3[nH]nc(N)c3c2)ccn1]

A solution of methyl (4-(3-amino-7-bromo-1H-indazol-5-yl)pyridin-2-yl)carbamate (1.1 g, 2.96 mmol, 1 Eq), (4-(difluoromethyl)phenyl)boronic acid (0.76 g, 4.44 mmol, 1.5 Eq), cesium carbonate (2.9 g, 8.88 mmol, 3 Eq) and [1,1′-bis(diphenylphosphino)ferrocene]dichloropalladium(II) (322 mg, 0.44 mmol, 0.15 Eq) in dioxane (8 mL) and water (2 mL) was stirred under nitrogen atmosphere at 80° C. for 1 h. The reaction mixture was poured onto cold water (10 mL). The produced ppt was filtered, washed with water (5 mL), dried and purified using column chromatography (5% MeOH in EtOAc) to give the titled product as white solid (0.7 g, 1.7 mmol, 58%). 1H NMR (500 MHz, DMSO) δ 11.81 (s, 1H), 10.23 (s, 1H), 8.32 (d, J=5.2 Hz, 1H), 8.24 (s, 2H), 7.91 (d, J=7.8 Hz, 2H), 7.83-7.64 (m, 3H), 7.47 (d, J=5.2 Hz, 1H), 7.15 (t, J=55.9 Hz, 1H), 5.67 (s, 2H), 3.72 (s, 3H). LC-MS: For C21H17F2N5O2 requires 409.40 found 410.2 (M+H).

### Example 282

### N-(4-(3-Amino-7-(3,3-dimethylbut-1-yn-1-yl)-1H-indazol-5-yl)pyridin-2-yl)oxazol-2-amine

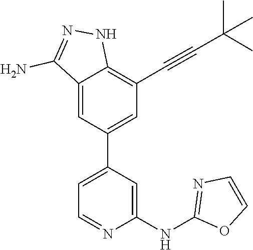 [SMILES: CC(C)(C)C#Cc1cc(-c2ccnc(Nc3ncco3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), N-(4-chloropyridin-2-yl)oxazol-2-amine (0.34 g, 1.76 mmol, 1.2 Eq), cesium carbonate (1 g, 2.94 mmol, 2 Eq) and 1,1′-bis(di-tert-butylphosphino) ferrocene palladium chloride (144 mg, 0.22 mmol, 0.15 Eq) in dioxane (4 mL) and water (1 mL) was heated in the microwave at 120° C. for 4 h. The reaction mixture was cooled and extracted between EtOAc (10 mL) and water (5 mL). The organic layer was washed with brine (2×5 mL), dried over anhydrous Na₂SO₄ and evaporated under reduced pressure.

The crude residue was purified using column chromatography (90% EtOAc in petroleum ether) followed by HPLC purification (60% MeCN in H₂O) to give the titled product as yellow solid (186 mg, 0.5 mmol, 35%). ¹H NMR (500 MHz, DMSO-d₆) δ 11.84 (s, 1H), 10.74 (s, 1H), 8.32-8.26 (m, 2H), 8.15 (d, J=1.7 Hz, 1H), 7.73 (s, 1H), 7.53 (d, J=1.7 Hz, 1H), 7.26 (d, J=5.3 Hz, 1H), 7.13-7.09 (m, 1H), 5.64 (s, 2H), 1.38 (s, 9H). LRMS: Calculated for C₂₁H₂₀N₆O requires 372.43 found 373.3 (M+H).

### Route to Example 282

### N-(4-Chloropyridin-2-yl)oxazol-2-amine

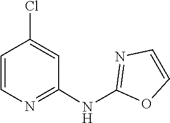 [SMILES: Clc1ccnc(Nc2ncco2)c1]

A mixture of palladium(II) acetate (67 mg, 0.3 mmol, 0.05 Eq) and xantphos (335 mg, 0.6 mmol, 0.1 Eq) in anhydrous toluene (1 mL) was stirred under nitrogen atmosphere for 15 min. The resulting mixture was added to a mixture of 2-bromo-4-chloropyridine (1.7 g, 8.92 mmol, 1.5 Eq), oxazol-2-amine (0.5 g, 5.95 mmol, 1 Eq) and Cs2CO3 (3.9 g, 11.9 mmol, 2 Eq) in toluene (2 mL) under nitrogen atmosphere. The reaction mixture was allowed to stir at 110° C. for 30 h. The organic solvent was removed under reduced pressure. The crude residue was stirred in MeOH (10 mL) for 30 min., filtered and washed with MeOH (2×3 mL). The filtrate was concentrated under reduced pressure. The crude residue was purified by column chromatography (20% EtOAc in petroleum ether) to give the tilted product as white solid (350 mg, 1.8 mmol, 30%). ¹H NMR (500 MHz, DMSO-d₆) δ 7.06-7.12 (m, 2H), 7.75 (d, J=1.0 Hz, 1H), 8.18 (d, J=2.0 Hz, 1H), 8.25 (d, J=5.4 Hz, 1H), 11.07 (s, 1H). LRMS: Calculated for C₈H₆ClN₃O requires 195.61 found 196.1 (M+H).

### Example 283

### 7-(3,3-Dimethylbut-1-yn-1-yl)-5-(2-((3,3,3-trifluoropropyl)amino)pyridin-4-yl)-1H-indazol-3-amine

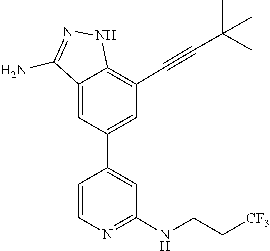 [SMILES: CC(C)(C)C#Cc1cc(-c2ccnc(NCCC(F)(F)F)c2)cc2c(N)n[nH]c12]

3,3-Dimethylbut-1-yne (204 mg, 0.3 mL, 2.48 mmol, 2 Eq) was added to a solution of 7-bromo-5-(2-((3,3,3-trifluoropropyl)amino)pyridin-4-yl)-1H-indazol-3-amine (0.5 g, 1.24 mmol, 1 Eq), copper(I) iodide (47 mg, 0.24 mmol, 0.2 Eq) and [1,1′-bis(diphenylphosphino)ferrocene]dichloropalladium(II) (91 mg, 0.13 mmol, 0.1 Eq) in anhydrous DMF (2 mL) and triethylamine (2 mL) under nitrogen atmosphere. The reaction mixture was allowed to stir at 60° C. for 18 h. The reaction mixture was extracted between EtOAc (10 mL) and water (5 mL). The organic layer was washed with brine (2×5 mL), dried over anhydrous Na₂SO₄ and evaporated under reduced pressure. The crude residue was purified using column chromatography (70% EtOAc in petroleum ether) followed by HPLC purification (60% MeCN in H₂O) to give the titled product as white solid (80 mg, 0.2 mmol, 16%). ¹H NMR (500 MHz, DMSO) δ 1.37 (s, 9H), 2.56 (dt, J=11.6, 7.0 Hz, 2H), 3.56 (q, J=6.7 Hz, 2H), 5.58 (s, 2H), 6.70 (t, J=5.8 Hz, 1H), 6.77 (d, J=1.6 Hz, 1H), 6.85 (dd, J=5.3, 1.6 Hz, 1H), 7.49 (d, J=1.6 Hz, 1H), 8.05 (d, J=5.4 Hz, 1H), 8.08 (d, J=1.6 Hz, 1H), 11.77 (s, 1H). LRMS: Calculated for C21H22F3N5 requires 401.44 found 402.3 (M+H).

### Route to Example 283

### 4-Iodo-N-(3,3,3-trifluoropropyl)pyridin-2-amine

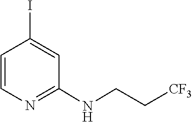 [SMILES: FC(F)(F)CCNc1cc(I)ccn1]

A solution of 2-fluoro-4-iodopyridine (1 g, 4.49 mmol, 1 Eq), 3,3,3-trifluoropropan-1-amine (1 g, 0.8 mL, 8.98 mmol, 2 Eq) and triethylamine (1.4 g, 2 mL, 13.47 mmol, 3 Eq) in DMSO (5 mL) was stirred in a sealed vial (20 mL) at 100° C. for 18 h. The reaction mixture was cooled and extracted between EtOAc (10 mL) and water (5 mL). The organic layer was washed with brine (3×5 mL), dried over anhydrous Na₂SO₄ and evaporated under reduced pressure. The crude residue was purified through column chromatography (20% EtOAc in petroleum ether) to give the titled product as white solid (0.8 g, 2.5 mmol, 56%). ¹H NMR (500 MHz, DMSO) δ 2.45-2.56 (m, 2H), 3.47 (td, J=7.0, 5.8 Hz, 2H), 6.84-6.92 (m, 2H), 6.94 (d, J=1.4 Hz, 1H), 7.72 (d, J=5.3 Hz, 1H).

### 7-Bromo-5-(2-((3,3,3-trifluoropropyl)amino)pyridin-4-yl)-1H-indazol-3-amine

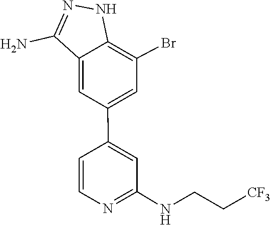 [SMILES: Nc1n[nH]c2c(Br)cc(-c3ccnc(NCCC(F)(F)F)c3)cc12]

A solution of 7-bromo-5-(4,4,5,5-tetramethyl-1,3,2-dioxaborolan-2-yl)-1H-indazol-3-amine (1 g, 2.96 mmol, 1 Eq), 4-iodo-N-(3,3,3-trifluoropropyl)pyridin-2-amine (0.94 g, 2.96 mmol, 1 Eq), cesium carbonate (2.9 g, 8.88 mmol, 3 Eq) and bis(triphenylphosphine)palladium chloride (312 mg, 0.44 mmol, 0.15 Eq) in dioxane (8 mL) and water (2 mL) was stirred under nitrogen atmosphere at 80° C. for 18 h. Cold water (10 mL) was added to the reaction mixture. The produced precipitate was filtered and washed with cold water (2×5 mL). The crude product was filtered through silica and dried to be used in the next step without further purification (0.5 g, 1.25 mmol, 42%).

### Example 284

### 7-(Cyclopropylethynyl)-5-(2-(oxetan-3-ylamino)pyridin-4-yl)-1H-indazol-3-amine SU1719

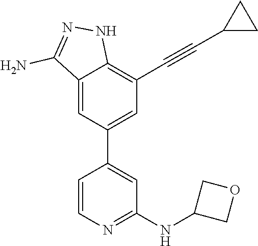 [SMILES: Nc1n[nH]c2c(C#CC3CC3)cc(-c3ccnc(NC4COC4)c3)cc12]

To a solution of 7-(cyclopropylethynyl)-5-(4,4,5,5-tetramethyl-1,3,2-dioxaborolan-2-yl)-1H-indazol-3-amine (70 mg, 0.216 mmol, 1.1 Eq) in a degassed 9:1 solution of dioxane/water (5 mL), were added 4-iodo-N-(oxetan-3-yl)pyridin-2-amine (54.35 mg, 0.196 mmol, 1 Eq), cesium carbonate (191.58 mg, 0.588 mmol, 3 Eq) and bis(triphenylphosphine)palladium chloride (14.74 mg, 0.021 mmol, 0.1 Eq) under argon atmosphere. The resulting reaction mixture was allowed to stir at 70° C. overnight. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (135.39 mg, 0.39 mmol, 50%). ¹H NMR (500 MHz, DMSO-d₆) δ 11.82 (s, 1H), 8.05 (d, J=1.6 Hz, 1H), 8.00 (d, J=5.4 Hz, 1H), 7.52 (d, J=1.6 Hz, 1H), 7.23 (d, J=6.3 Hz, 1H), 6.85 (dd, J=5.4, 1.6 Hz, 1H), 6.73 (s, 1H), 5.56 (s, 2H), 4.96 (q, J=6.6 Hz, 1H), 4.84 (t, J=6.7 Hz, 2H), 4.47 (t, J=6.2 Hz, 2H), 1.62 (tt, J=7.9, 5.1 Hz, 1H), 0.95-0.90 (m, 4H) ppm. LRMS (ESI+ve): Calculated for C₂₀H₁₉N₅O requires 345.41 found 346.2 (M+H).

### Route to Example 284

### 4-Iodo-N-(oxetan-3-yl)pyridin-2-amine

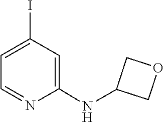 [SMILES: Ic1ccnc(NC2COC2)c1]

In a sealed 10 mL microwave vial, 2-fluoro-4-iodopyridine (200 mg, 0.89 mmol, 1 Eq) was dissolved in 3 mL of DMSO. Then 3-aminooxetane (97.57 mg, 0.936 mL, 1.78 mmol, 1.5 Eq) and triethylamine (270.17 mg, 0.372 mL, 2.67 mmol, 3 Eq) were added and the resulting reaction mixture was heated at 100° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (135.14 mg, 0.49 mmol, 55%). ¹H NMR (500 MHz, DMSO-d₆) δ 7.67 (d, J=5.3 Hz, 1H), 7.39 (d, J=6.0 Hz, 1H), 6.95-6.83 (m, 2H), 4.88-4.80 (m, 1H), 4.77 (dd, J=7.3, 5.8 Hz, 2H), 4.40 (t, J=6.1 Hz, 2H). ppm.

### 7-(Cyclopropylethynyl)-5-(4,4,5,5-tetramethyl-1,3,2-dioxaborolan-2-yl)-1H-indazol-3-amine

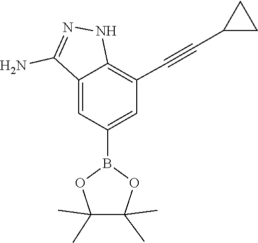 [SMILES: CC1(C)OB(c2cc(C#CC3CC3)c3[nH]nc(N)c3c2)OC1(C)C]

A solution of 7-bromo-5-(4,4,5,5-tetramethyl-1,3,2-dioxaborolan-2-yl)-1H-indazol-3-amine (1.00 g, 3.0 mmol, 1 Eq), copper (1) iodide (0.100 g, 0.5 mmol, 0.15 Eq) and bis(triphenylphosphine) palladium(II) chloride (0.220 g, 0.3 mmol, 0.1 Eq) in 10 ml of N,N-dimethyformamide-triethylamine (4:1) was degassed in sealed tube followed by addition of ethynylcyclopropane (1.01 mL, 12 mmol, 4 Eq). The reaction mixture was heated to 80° C. for 1.2 h. The resulting mixture was diluted with EtOAc, filtered through silica, concentrated under reduced pressure and the residue was purified by column chromatography (60% EtOAc in petroleum ether) to give the product as a brown foam (0.563 g, 1.65 mmol, 55%). ¹H NMR (500 MHz, DMSO-d₆): δ 11.77 (s, 1H), 8.11 (s, 1H), 7.44 (s, 1H), 5.56 (s, 2H), 1.57 (ddd, J=13.0, 8.1, 5.2 Hz, 1H), 1.29 (s, 12H), 0.90-0.85 (m, 4H) ppm. LRMS: Calculated for C₁₈H₂₂BN₃O₂ requires 323.2 found 324.3 (M+1).

### Example 285

### Methyl (4-(3-amino-7-(3,3-dimethylbut-1-yn-1-yl)-1H-indazol-5-yl)pyridin-2-yl)carbamate

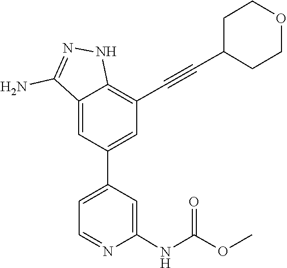 [SMILES: COC(=O)Nc1cc(-c2cc(C#CC3CCOCC3)c3[nH]nc(N)c3c2)ccn1]

To a solution of 7-((tetrahydro-2H-pyran-4-yl)ethynyl)-5-(4,4,5,5-tetramethyl-1,3,2-dioxaborolan-2-yl)-1H-indazol-3-amine (116.23 mg, 0.316 mmol, 1.1 Eq) in a degassed 9:1 solution of dioxane/water (5 mL), were added methyl (4-iodopyridin-2-yl)carbamate (80 mg, 0.287 mmol, 1 Eq), cesium carbonate (280.53 mg, 0.861 mmol, 3 Eq) and bis(triphenylphosphine)palladium chloride (20.14 mg, 0.028 mmol, 0.1 Eq) under argon atmosphere. The resulting reaction mixture was allowed to stir at 70° C. overnight. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a white solid (73.02 mg, 0.19 mmol, 65%). ¹H NMR (500 MHz, DMSO-d₆) δ 11.90 (s, 1H), 10.22 (s, 1H), 8.30 (d, J=5.3 Hz, 1H), 8.18 (d, J=1.6 Hz, 1H), 8.15 (s, 1H), 7.60 (d, J=1.6 Hz, 1H), 7.37 (dd, J=5.4, 1.8 Hz, 1H), 5.66 (s, 2H), 3.88 (dt, J=11.6, 4.3 Hz, 2H), 3.72 (s, 3H), 3.49 (td, J=9.0, 4.6 Hz, 2H), 2.99 (tt, J=8.8, 4.1 Hz, 1H), 1.92 (dq, J=12.4, 4.0 Hz, 2H), 1.76 (dtd, J=12.9, 9.1, 3.7 Hz, 2H) ppm. LRMS (ESI+ve): Calculated for C₂₁H₂₁N₅O₃ requires 391.43 found 392.2 (M+H).

### Route to Example 285

### Methyl (4-iodopyridin-2-yl)carbamate

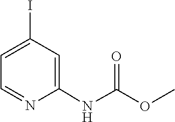

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise methyl chloroformate (257.71 mg, 0.21 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (20% EtOAc in petroleum ether) to give the titled product as a white solid (523.85 mg, 1.88 mmol, 83%). ¹H NMR (500 MHz, DMSO-d₆) δ 10.33 (s, 1H), 8.26 (d, J=1.4 Hz, 1H), 7.98 (d, J=5.2 Hz, 1H), 7.45 (dd, J=5.2, 1.5 Hz, 1H), 3.68 (s, 3H) ppm.

### 7-((Tetrahydro-2H-pyran-4-yl)ethynyl)-5-(4,4,5,5-tetramethyl-1,3,2-dioxaborolan-2-yl)-1H-indazol-3-amine

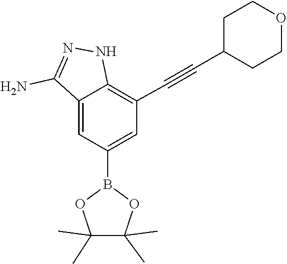 [SMILES: CC1(C)OB(c2cc(C#CC3CCOCC3)c3[nH]nc(N)c3c2)OC1(C)C]

A solution of 7-bromo-5-(4,4,5,5-tetramethyl-1,3,2-dioxaborolan-2-yl)-1H-indazol-3-amine (200 mg, 0.59 mmol, 1 Eq), copper (1) iodide (23 mg, 0.12 mmol, 0.2 Eq) and [1,1′-bis(diphenylphosphino)ferrocene]palladium(II) dichloride (43 mg, 0.06 mmol, 0.1 Eq) in 2 ml of N,N-dimethyformamide-triethylamine (1:1) was degassed in sealed tube followed by addition of 4-ethynyltetrahydro-2H-pyran (0.2 mL, 1.18 mmol, 2 Eq). The reaction mixture was heated to 80° C. for 2 h. The reaction was then diluted with EtOAc (30 mL) and the organic layer was washed with water (2×5 mL), dried over anhydrous Na₂SO₄ and evaporated under reduced pressure. The crude residue was purified by column chromatography (80% EtOAc in petroleum ether) to give the titled product as brown crystals (100 mg, 0.27 mmol, 46%). ¹H NMR (500 MHz, DMSO-d₆): δ 1.30 (s, 12H), 1.72 (ddt, J=13.7, 9.2, 4.5 Hz, 2H), 1.84-1.94 (m, 2H), 2.94 (tt, J=8.7, 4.1 Hz, 1H), 3.48 (ddd, J=11.6, 8.9, 2.9 Hz, 2H), 3.85 (dt, J=11.5, 4.3 Hz, 2H), 5.61 (s, 2H), 7.50 (s, 1H), 8.16 (s, 1H), 11.81 (s, 1H). LRMS (ESI+ve): Calculated for C₂₀H₂₆BN₃O₃ requires 367.26 found 368.3 (M+H).

### Example 286

### Methyl (4-(3-amino-7-((3-methyloxetan-3-yl)ethynyl)-1H-indazol-5-yl)pyridin-2-yl)carbamate

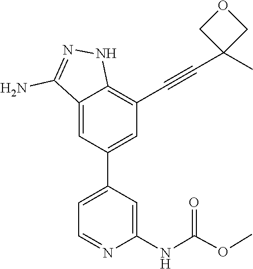 [SMILES: COC(=O)Nc1cc(-c2cc(C#CC3(C)COC3)c3[nH]nc(N)c3c2)ccn1]

7-((3-Methyloxetan-3-yl)ethynyl)-5-(4,4,5,5-tetramethyl-1,3,2-dioxaborolan-2-yl)-1H-indazol-3-amine (0.5 g, 1.4 mmol, 1 Eq), methyl (4-iodopyridin-2-yl)carbamate (0.47 g, 1.68 mmol, 1.2 Eq), cesium carbonate (1.4 g, 4.2 mmol, 3 Eq) and bis(triphenylphosphine)palladium chloride (148 mg, 0.21 mmol, 0.15 Eq) were added to the crude residue. The mixture was dissolved in dioxane (4 mL) and water (1 mL) and stirred under nitrogen atmosphere at 80° C. for 18 h. The reaction mixture was poured into ice-cold water (10 mL). The precipitate was filtered, washed with water (2×5 mL) and dried. The crude solid was purified by column chromatography (C18, 60% MeCN in water) to give the titled product as yellow solid (150 mg, 0.4 mmol, 28%). 1H NMR (500 MHz, DMSO) δ 12.00 (s, 1H), 10.23 (s, 1H), 8.30 (d, J=5.3 Hz, 1H), 8.21 (d, J=1.7 Hz, 1H), 8.16 (d, J=1.7 Hz, 1H), 7.63 (d, J=1.7 Hz, 1H), 7.38 (dd, J=5.3, 1.7 Hz, 1H), 5.69 (s, 2H), 4.93 (d, J=5.4 Hz, 2H), 4.46 (d, J=5.4 Hz, 2H), 3.72 (s, 3H), 1.73 (s, 3H). LRMS: For C20H19N5O3 requires 377.4 found 378.3 (M+H).

### Route to Example 286

### 7-((3-Methyloxetan-3-yl)ethynyl)-5-(4,4,5,5-tetramethyl-1,3,2-dioxaborolan-2-yl)-1H-indazol-3-amine

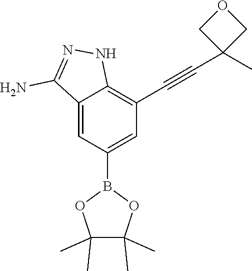 [SMILES: CC1(C#Cc2cc(B3OC(C)(C)C(C)(C)O3)cc3c(N)n[nH]c23)COC1]

3-Ethynyl-3-methyloxetane (114 mg, 0.15 mL, 1.18 mmol, 2 Eq) was added to a solution of 7-bromo-5-(4,4,5,5-tetramethyl-1,3,2-dioxaborolan-2-yl)-1H-indazol-3-amine (200 mg, 0.59 mmol, 1 Eq), copper(I) iodide (23 mg, 0.12 mmol, 0.2 Eq) and [1,1′-bis(diphenylphosphino)ferrocene]palladium(II) dichloride (43 mg, 0.06 mmol, 0.1 Eq) in anhydrous DMF (1 mL) and triethylamine (1 mL) under nitrogen atmosphere. The reaction mixture was allowed to stir at 80° C. for 2 h. The reaction mixture was cooled, extracted between EtOAc (30 mL) and water (10 mL). The organic layer was washed with brine (2×10 mL), dried over anhydrous sodium sulfate and removed under reduced pressure. The crude residue was loaded onto silica gel and purified by column chromatography (80% EtOAc in petroleum ether) to give the titled product as brown crystals (50 mg, 0.15 mmol, 25%). ¹H NMR (500 MHz, DMSO) δ 1.31 (s, 12H), 1.69 (s, 3H), 4.44 (d, J=5.3 Hz, 2H), 4.88 (d, J=5.3 Hz, 2H), 5.64 (s, 2H), 7.52 (s, 1H), 8.18 (s, 1H), 11.90 (s, 1H). LC-MS: For C19H24BN3O3 requires 353.23 found 354.2 (M+H).

### Example 287

### 4-(3-Amino-5-(2-(oxetan-3-ylamino)pyridin-4-yl)-1H-indazol-7-yl)-2-methylbut-3-yn-2-ol

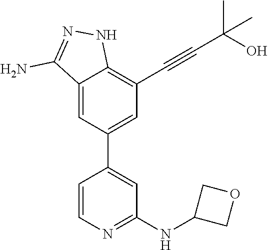 [SMILES: CC(C)(O)C#Cc1cc(-c2ccnc(NC3COC3)c2)cc2c(N)n[nH]c12]

To a solution of 4-(3-amino-5-(4,4,5,5-tetramethyl-1,3,2-dioxaborolan-2-yl)-1H-indazol-7-yl)-2-methylbut-3-yn-2-ol (135.95 mg, 0.40 mmol, 1.1 Eq) in a degassed 9:1 solution of dioxane/water (5 mL), were added 4-iodo-N-(oxetan-3-yl)pyridin-2-amine (100 mg, 0.36 mmol, 1 Eq), cesium carbonate (390.98 mg, 1.2 mmol, 3 Eq) and bis(triphenylphosphine)palladium chloride (28.07 mg, 0.04 mmol, 0.1 Eq) under argon atmosphere. The resulting reaction mixture was allowed to stir at 70° C. overnight. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (91.58 mg, 0.252 mmol, 70%). ¹H NMR (500 MHz, DMSO-d₆) δ 11.78 (s, 1H), 8.10 (s, 1H), 8.00 (d, J=5.4 Hz, 1H), 7.52 (s, 1H), 7.23 (d, J=6.2 Hz, 1H), 6.86 (dd, J=5.5, 1.6 Hz, 1H), 6.74 (d, J=1.6 Hz, 1H), 5.61 (s, 2H), 5.43 (s, 1H), 4.94 (p, J=6.7 Hz, 1H), 4.83 (t, J=6.7 Hz, 2H), 4.46 (t, J=6.2 Hz, 2H), 1.54 (s, 6H) ppm. LRMS (ESI+ve): Calculated for C₂₀H₂₁N₅O₂ requires 363.42 found 364.3 (M+H).

### Route to Example 287

### 4-Iodo-N-(oxetan-3-yl)pyridin-2-amine

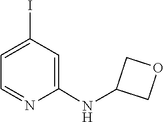 [SMILES: Ic1ccnc(NC2COC2)c1]

In a sealed 10 mL microwave vial, 2-fluoro-4-iodopyridine (200 mg, 0.89 mmol, 1 Eq) was dissolved in 3 mL of DMSO. Then 3-aminooxetane (97.57 mg, 0.936 mL, 1.78 mmol, 1.5 Eq) and triethylamine (270.17 mg, 0.372 mL, 2.67 mmol, 3 Eq) were added and the resulting reaction mixture was heated at 100° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (135.14 mg, 0.49 mmol, 55%). ¹H NMR (500 MHz, DMSO-d₆) δ 7.67 (d, J=5.3 Hz, 1H), 7.39 (d, J=6.0 Hz, 1H), 6.95-6.83 (m, 2H), 4.88-4.80 (m, 1H), 4.77 (dd, J=7.3, 5.8 Hz, 2H), 4.40 (t, J=6.1 Hz, 2H). ppm.

### 4-(3-amino-5-(4,4,5,5-tetramethyl-1,3,2-dioxaborolan-2-yl)-1H-indazol-7-yl)-2-methylbut-3-yn-2-ol

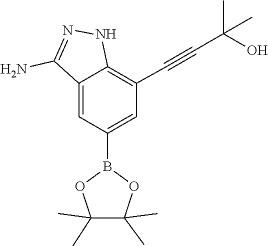 [SMILES: CC(C)(O)C#Cc1cc(B2OC(C)(C)C(C)(C)O2)cc2c(N)n[nH]c12]

A solution of 7-bromo-5-(4,4,5,5-tetramethyl-1,3,2-dioxaborolan-2-yl)-1H-indazol-3-amine (1.00 g, 3.0 mmol, 1 Eq), copper (1) iodide (0.100 g, 0.5 mmol, 0.15 Eq) and bis(triphenylphosphine) palladium(II) chloride (0.220 g, 0.3 mmol, 0.1 Eq) in 10 ml of N,N-dimethyformamide-triethylamine (4:1) was degassed in sealed tube followed by addition of 2-methylbut-3-yn-2-ol (1.16 mL, 12 mmol, 4 Eq). The reaction mixture was heated to 80° C. for 1.2 h. The resulting mixture was diluted with EtOAc, filtered through silica, concentrated under reduced pressure and the residue was purified by column chromatography (70% EtOAc in petroleum ether) to give the product as a brown foam (0.563 g, 1.65 mmol, 55%). ¹H NMR (500 MHz, DMSO-d₆): δ 11.75 (s, 1H), 8.15 (s, 1H), 7.48 (s, 1H), 5.62 (s, 2H), 5.35 (s, 1H), 1.51 (s, 6H), 1.30 (s, 12H) ppm. LRMS: Calculated for C₁₈H₂₄BN₃O₃ requires 341.2 found 342.2 (M+1).

### Example 288

### 5-(2-(Oxetan-3-ylamino)pyridin-4-yl)-7-((tetrahydro-2H-pyran-4-yl)ethynyl)-1H-indazol-3-amine

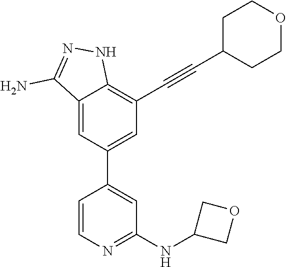 [SMILES: Nc1n[nH]c2c(C#CC3CCOCC3)cc(-c3ccnc(NC4COC4)c3)cc12]

To a solution of 7-((tetrahydro-2H-pyran-4-yl)ethynyl)-5-(4,4,5,5-tetramethyl-1,3,2-dioxaborolan-2-yl)-1H-indazol-3-amine (73.16 mg, 0.199 mmol, 1.1 Eq) in a degassed 9:1 solution of dioxane/water (5 mL), were added 4-iodo-N-(oxetan-3-yl)pyridin-2-amine (50 mg, 0.181 mmol, 1 Eq), cesium carbonate (176.92 mg, 0.543 mmol, 3 Eq) and bis(triphenylphosphine)palladium chloride (12.63 mg, 0.018 mmol, 0.1 Eq) under argon atmosphere. The resulting reaction mixture was allowed to stir at 70° C. overnight. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether, then 10% MeOH in EtOAc) followed by HPLC purification to afford the titled compound as a brown solid (31.72 mg, 0.08 mmol, 45%). ¹H NMR (500 MHz, DMSO-d₆) δ 11.83 (s, 1H), 8.08 (d, J=1.7 Hz, 1H), 7.99 (d, J=5.3 Hz, 1H), 7.54 (d, J=1.6 Hz, 1H), 7.23 (d, J=6.2 Hz, 1H), 6.85 (dd, J=5.5, 1.7 Hz, 1H), 6.74 (d, J=1.6 Hz, 1H), 5.58 (s, 2H), 4.94 (p, J=6.6 Hz, 1H), 4.83 (t, J=6.6 Hz, 2H), 4.46 (t, J=6.2 Hz, 2H), 3.86 (dt, J=11.5, 4.2 Hz, 2H), 3.47 (ddd, J=11.7, 9.1, 2.8 Hz, 3H), 2.96 (tt, J=8.7, 4.0 Hz, 1H), 1.90 (dq, J=12.5, 3.9 Hz, 2H), 1.73 (ddt, J=13.8, 9.3, 4.6 Hz, 3H) ppm. LRMS (ESI+ve): Calculated for C₂₂H₂₃N₅O₂ requires 389.46 found 390.3 (M+H).

### Route to Example 288

### 4-Iodo-N-(oxetan-3-yl)pyridin-2-amine

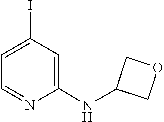 [SMILES: Ic1ccnc(NC2COC2)c1]

In a sealed 10 mL microwave vial, 2-fluoro-4-iodopyridine (200 mg, 0.89 mmol, 1 Eq) was dissolved in 3 mL of DMSO. Then 3-aminooxetane (97.57 mg, 0.936 mL, 1.78 mmol, 1.5 Eq) and triethylamine (270.17 mg, 0.372 mL, 2.67 mmol, 3 Eq) were added and the resulting reaction mixture was heated at 100° C. under microwave irradiation for 4 hours.

The reaction was then diluted with EtOAc (10 mL) and the organic layer was washed with water (2×5 mL), dried over anhydrous Na₂SO₄ and evaporated under reduced pressure. The crude residue was purified by column chromatography (30% EtOAc in petroleum ether) to give the titled product (135.14 mg, 0.49 mmol, 55%). ¹H NMR (500 MHz, DMSO-d₆) δ 7.67 (d, J=5.3 Hz, 1H), 7.39 (d, J=6.0 Hz, 1H), 6.95-6.83 (m, 2H), 4.88-4.80 (m, 1H), 4.77 (dd, J=7.3, 5.8 Hz, 2H), 4.40 (t, J=6.1 Hz, 2H). ppm.

### 7-((Tetrahydro-2H-pyran-4-yl)ethynyl)-5-(4,4,5,5-tetramethyl-1,3,2-dioxaborolan-2-yl)-1H-indazol-3-amine

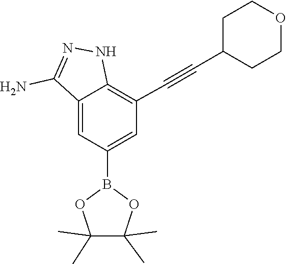 [SMILES: CC1(C)OB(c2cc(C#CC3CCOCC3)c3[nH]nc(N)c3c2)OC1(C)C]

A solution of 7-bromo-5-(4,4,5,5-tetramethyl-1,3,2-dioxaborolan-2-yl)-1H-indazol-3-amine (200 mg, 0.59 mmol, 1 Eq), copper (I) iodide (23 mg, 0.12 mmol, 0.2 Eq) and [1,1′-bis(diphenylphosphino)ferrocene]palladium(II) dichloride (43 mg, 0.06 mmol, 0.1 Eq) in 2 ml of N,N-dimethyformamide-triethylamine (1:1) was degassed in sealed tube followed by addition of 4-ethynyltetrahydro-2H-pyran (0.2 mL, 1.18 mmol, 2 Eq). The reaction mixture was heated to 80° C. for 2 h. The reaction was then diluted with EtOAc (30 mL) and the organic layer was washed with water (2×5 mL), dried over anhydrous Na₂SO₄ and evaporated under reduced pressure. The crude residue was purified by column chromatography (80% EtOAc in petroleum ether) to give the titled product as brown crystals (100 mg, 0.27 mmol, 46%). ¹H NMR (500 MHz, DMSO-d₆): δ 1.30 (s, 12H), 1.72 (ddt, J=13.7, 9.2, 4.5 Hz, 2H), 1.84-1.94 (m, 2H), 2.94 (tt, J=8.7, 4.1 Hz, 1H), 3.48 (ddd, J=11.6, 8.9, 2.9 Hz, 2H), 3.85 (dt, J=11.5, 4.3 Hz, 2H), 5.61 (s, 2H), 7.50 (s, 1H), 8.16 (s, 1H), 11.81 (s, 1H). LRMS (ESI+ve): Calculated for C₂₀H₂₆BN₃O₃ requires 367.26 found 368.3 (M+H).

### Example 289

### N-(4-(3-Amino-7-(cyclopropylethynyl)-1H-indazol-5-yl)pyridin-2-yl)cyclopropanecarboxamide

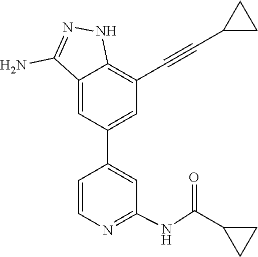 [SMILES: Nc1n[nH]c2c(C#CC3CC3)cc(-c3ccnc(NC(=O)C4CC4)c3)cc12]

To a solution of 7-(cyclopropylethynyl)-5-(4,4,5,5-tetramethyl-1,3,2-dioxaborolan-2-yl)-1H-indazol-3-amine (213.31 mg, 0.66 mmol, 1.1 Eq) in a degassed 9:1 solution of dioxane/water (5 mL), were added N-(4-iodopyridin-2-yl)cyclopropanecarboxamide (151.84 mg, 0.60 mmol, 1 Eq), cesium carbonate (586.47 mg, 1.8 mmol, 3 Eq) and bis(triphenylphosphine)palladium chloride (42.11 mg, 0.06 mmol, 0.1 Eq) under argon atmosphere. The resulting reaction mixture was allowed to stir at 70° C. overnight. The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (171.56 mg, 0.48 mmol, 80%). ¹H NMR (500 MHz, DMSO-d₆) δ 11.85 (s, 1H), 10.82 (s, 1H), 8.40 (d, J=1.6 Hz, 1H), 8.32 (d, J=5.3 Hz, 1H), 8.13 (d, J=1.6 Hz, 1H), 7.54 (d, J=1.6 Hz, 1H), 7.37 (dd, J=5.3, 1.8 Hz, 1H), 5.61 (s, 2H), 2.05 (tt, J=7.8, 4.8 Hz, 1H), 1.61 (tt, J=7.5, 5.6 Hz, 1H), 0.93-0.89 (m, 4H), 0.86-0.80 (m, 4H) ppm. LRMS (ESI+ve): Calculated for C₂₁H₁₉N₅O requires 357.42 found 358.3 (M+H).

### Route to Example 289

### N-(4-Iodopyridin-2-yl)cyclopropanecarboxamide

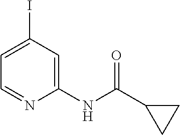 [SMILES: O=C(Nc1cc(I)ccn1)C1CC1]

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise cyclopropanecarbonyl chloride (285.07 mg, 0.247 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (30% EtOAc in petroleum ether) to give the titled product (622 mg, 2.16 mmol, 95%). ¹H NMR (500 MHz, DMSO-d₆) δ 10.90 (s, 1H), 8.54 (d, J=1.2 Hz, 1H), 8.04 (d, J=5.2 Hz, 1H), 7.49 (dd, J=5.2, 1.5 Hz, 1H), 2.00 (quin, J=6.2 Hz, 1H), 0.84 (s, 2H), 0.82 (s, 2H) ppm.

### 7-(Cyclopropylethynyl)-5-(4,4,5,5-tetramethyl-1,3,2-dioxaborolan-2-yl)-1H-indazol-3-amine

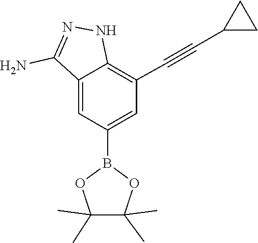 [SMILES: CC1(C)OB(c2cc(C#CC3CC3)c3[nH]nc(N)c3c2)OC1(C)C]

A solution of 7-bromo-5-(4,4,5,5-tetramethyl-1,3,2-dioxaborolan-2-yl)-1H-indazol-3-amine (1.00 g, 3.0 mmol, 1 Eq), copper (1) iodide (0.100 g, 0.5 mmol, 0.15 Eq) and bis(triphenylphosphine) palladium(II) chloride (0.220 g, 0.3 mmol, 0.1 Eq) in 10 ml of N,N-dimethyformamide-triethylamine (4:1) was degassed in sealed tube followed by addition of ethynylcyclopropane (1.01 mL, 12 mmol, 4 Eq). The reaction mixture was heated to 80° C. for 1.2 h. The resulting mixture was diluted with EtOAc, filtered through silica, concentrated under reduced pressure and the residue was purified by column chromatography (60% EtOAc in petroleum ether) to give the product as a brown foam (0.563 g, 1.65 mmol, 55%). ¹H NMR (500 MHz, DMSO-d₆): δ 11.77 (s, 1H), 8.11 (s, 1H), 7.44 (s, 1H), 5.56 (s, 2H), 1.57 (ddd, J=13.0, 8.1, 5.2 Hz, 1H), 1.29 (s, 12H), 0.90-0.85 (m, 4H) ppm. LRMS: Calculated for C₁₈H₂₂BN₃O₂ requires 323.2 found 324.3 (M+1).

### Example 290

### N-(4-(3-Amino-7-(3-hydroxy-3-methylbut-1-yn-1-yl)-1H-indazol-5-yl)pyridin-2-yl)cyclopropanecarboxamide

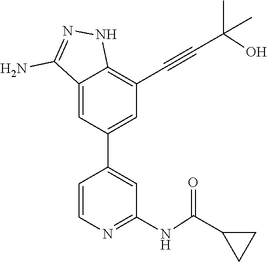 [SMILES: CC(C)(O)C#Cc1cc(-c2ccnc(NC(=O)C3CC3)c2)cc2c(N)n[nH]c12]

To a solution of 4-(3-amino-5-(4,4,5,5-tetramethyl-1,3,2-dioxaborolan-2-yl)-1H-indazol-7-yl)-2-methylbut-3-yn-2-ol (260 mg, 0.76 mmol, 1.1 Eq) in a degassed 9:1 solution of dioxane/water (5 mL), were added N-(4-iodopyridin-2-yl)cyclopropanecarboxamide (199.56 mg, 0.69 mmol, 1 Eq), cesium carbonate (674.44 mg, 2.07 mmol, 3 Eq) and bis(triphenylphosphine)palladium chloride (53.34 mg, 0.076 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (194.28 mg, 0.517 mmol, 75%). ¹H NMR (500 MHz, DMSO-d₆) δ 11.83 (s, 1H), 10.84 (s, 1H), 8.41 (d, J=1.7 Hz, 1H), 8.33 (d, J=5.2 Hz, 1H), 8.19 (d, J=1.7 Hz, 1H), 7.55 (d, J=1.6 Hz, 1H), 7.39 (dd, J=5.3, 1.7 Hz, 1H), 5.67 (s, 2H), 5.45 (s, 1H), 2.05 (td, J=7.8, 3.7 Hz, 1H), 0.83 (dtd, J=12.7, 6.4, 3.9 Hz, 4H) ppm. LRMS (ESI+ve): Calculated for C₂₁H₂₁N₅O₂ requires 375.43 found 376.2 (M+H).

### Route to Example 290

### N-(4-Iodopyridin-2-yl)cyclopropanecarboxamide

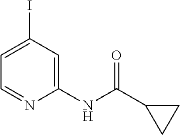 [SMILES: O=C(Nc1cc(I)ccn1)C1CC1]

To a solution of 4-iodopyridin-2-amine (500 mg, 2.27 mmol, 1 Eq) and triethylamine (1.38 g, 2 mL, 13.65 mmol, 1 Eq) in Dioxane (5 mL) was added dropwise cyclopropanecarbonyl chloride (285.07 mg, 0.247 mL, 2.73 mmol, 1.2 Eq) at 5° C. The reaction mixture was allowed to stir at room temperature overnight. The reaction was diluted with EtOAc (10 mL) and the organic layer was washed with water (2×5 mL), dried over anhydrous Na₂SO₄ and evaporated under reduced pressure. The crude residue was purified using column chromatography (30% EtOAc in petroleum ether) to give the titled product (622 mg, 2.16 mmol, 95%). ¹H NMR (500 MHz, DMSO-d₆) δ 10.90 (s, 1H), 8.54 (d, J=1.2 Hz, 1H), 8.04 (d, J=5.2 Hz, 1H), 7.49 (dd, J=5.2, 1.5 Hz, 1H), 2.00 (quin, J=6.2 Hz, 1H), 0.84 (s, 2H), 0.82 (s, 2H) ppm.

### 4-(3-Amino-5-(4,4,5,5-tetramethyl-1,3,2-dioxaborolan-2-yl)-1H-indazol-7-yl)-2-methylbut-3-yn-2-ol

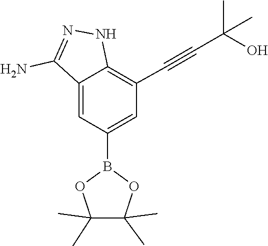 [SMILES: CC(C)(O)C#Cc1cc(B2OC(C)(C)C(C)(C)O2)cc2c(N)n[nH]c12]

A solution of 7-bromo-5-(4,4,5,5-tetramethyl-1,3,2-dioxaborolan-2-yl)-1H-indazol-3-amine (1.00 g, 3.0 mmol, 1 Eq), copper (1) iodide (0.100 g, 0.5 mmol, 0.15 Eq) and bis(triphenylphosphine) palladium(II) chloride (0.220 g, 0.3 mmol, 0.1 Eq) in 10 ml of N,N-dimethyformamide-triethylamine (4:1) was degassed in sealed tube followed by addition of 2-methylbut-3-yn-2-ol (1.16 mL, 12 mmol, 4 Eq). The reaction mixture was heated to 80° C. for 1.2 h. The resulting mixture was diluted with EtOAc, filtered through silica, concentrated under reduced pressure and the residue was purified by column chromatography (70% EtOAc in petroleum ether) to give the product as a brown foam (0.563 g, 1.65 mmol, 55%). ¹H NMR (500 MHz, DMSO-d₆): δ 11.75 (s, 1H), 8.15 (s, 1H), 7.48 (s, 1H), 5.62 (s, 2H), 5.35 (s, 1H), 1.51 (s, 6H), 1.30 (s, 12H) ppm. LRMS: Calculated for C₁₈H₂₄BN₃O₃ requires 341.2 found 342.2 (M+1).

### Example 291

### Methyl (4-(3-amino-7-(5-morpholinopent-1-yn-1-yl)-1H-indazol-5-yl)pyridin-2-yl)carbamate

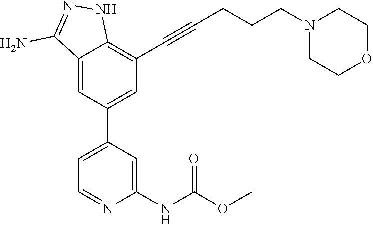 [SMILES: COC(=O)Nc1cc(-c2cc(C#CCCCN3CCOCC3)c3[nH]nc(N)c3c2)ccn1]

A mixture of 7-(5-chloropent-1-yn-1-yl)-5-(4,4,5,5-tetramethyl-1,3,2-dioxaborolan-2-yl)-1H-indazol-3-amine (0.5 g, 1.4 mmol, 1 Eq), morpholine (1.7 g, 1.7 mL, 19.6 mmol, 14 Eq) and potassium iodide (140 mg, 0.84 mmol, 0.6 Eq) in N,N-dimethylamide (2 mL) was stirred at 110° C. for 2 h. The reaction mixture was cooled and extracted between EtOAc (10 mL) and NaHCO₃ (5 mL). The organic layer was washed brine (3×10 mL), dried over anhydrous Na₂SO₄ and evaporated under reduced pressure. Methyl (4-iodopyridin-2-yl)carbamate (0.5 g, 1.7 mmol, 1.2 Eq), cesium carbonate (1.4 g, 4.2 mmol, 3 Eq) and bis(triphenylphosphine)palladium chloride (148 mg, 0.21 mmol, 0.15 Eq) were added to the crude residue. The mixture was dissolved in dioxane (4 mL) and water (1 mL) and stirred under nitrogen atmosphere at 80° C. for 18 h. The reaction mixture was poured into ice-cold water (10 mL). The precipitate was filtered, washed with water (2×5 mL) and dried. The crude solid was purified by column chromatography (40% MeOH in EtOAc) followed by HPLC purification to give the titled product as yellow solid (182 mg, 0.42 mmol, 30%). ¹H NMR (500 MHz, DMSO-d₆) 5 11.89 (s, 1H), 10.22 (s, 1H), 8.30 (d, J=5.3 Hz, 1H), 8.16 (d, J=8.0 Hz, 2H), 7.59 (s, 1H), 7.39-7.34 (m, 1H), 5.64 (s, 1H), 3.72 (s, 2H), 3.60 (t, J=4.6 Hz, 3H), 2.65 (s, 4H), 2.44 (t, J=7.1 Hz, 2H), 2.40 (s, 2H), 1.82 (q, J=7.2 Hz, 4H). LRMS: Calculated for C₂₃H₂₆N₆O₃ requires 434.50 found 435.3 (M+H).

### Route to Example 291

### 7-(5-Chloropent-1-yn-1-yl)-5-(4,4,5,5-tetramethyl-1,3,2-dioxaborolan-2-yl)-1H-indazol-3-amine

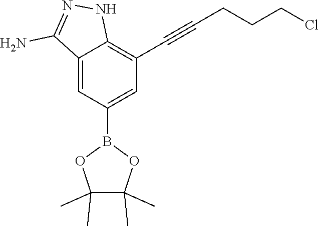 [SMILES: CC1(C)OB(c2cc(C#CCCCCl)c3[nH]nc(N)c3c2)OC1(C)C]

5-Chloropent-1-yne (121 mg, 0.15 mL, 1.18 mmol, 2 Eq) was added to a solution of 7-bromo-5-(4,4,5,5-tetramethyl-1,3,2-dioxaborolan-2-yl)-1H-indazol-3-amine (200 mg, 0.59 mmol, 1 Eq), copper(I) iodide (23 mg, 0.12 mmol, 0.2 Eq) and [1,1′-bis(diphenylphosphino)ferrocene]palladium(II) dichloride (43 mg, 0.06 mmol, 0.1 Eq) in anhydrous DMF (1 mL) and triethylamine (1 mL) under nitrogen atmosphere. The reaction mixture was allowed to stir at 80° C. for 2 h. The reaction mixture was cooled, extracted between EtOAc (30 mL) and water (10 mL). The organic layer was washed with brine (2×10 mL), dried over anhydrous sodium sulfate and removed under reduced pressure. The crude residue was loaded onto silica gel and purified by column chromatography (70% EtOAc in petroleum ether) to give the titled product as brown crystals (100 mg, 0.28 mmol, 47%). ¹H NMR (500 MHz, DMSO-d₆) δ 1.30 (s, 12H), 2.07 (p, J=6.7 Hz, 2H), 2.65 (t, J=6.9 Hz, 2H), 3.82 (t, J=6.4 Hz, 2H), 5.62 (s, 2H), 7.52 (s, 1H), 8.17 (s, 1H), 11.85 (s, 1H). LRMS: Calculated for C₁₈H₂₃BClN₃O₂ requires 359.66 found 360.1 (M+H).

### Example 292

### N-(4-(3-Amino-7-(3-hydroxy-3-methylbutyl)-1H-indazol-5-yl)pyridin-2-yl)cyclopropanecarboxamide

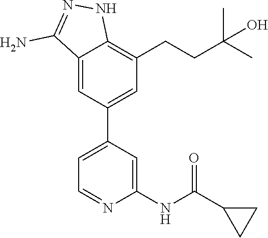 [SMILES: CC(C)(O)CCc1cc(-c2ccnc(NC(=O)C3CC3)c2)cc2c(N)n[nH]c12]

To a solution of N-(4-(3-amino-7-(3-hydroxy-3-methylbut-1-yn-1-yl)-1H-indazol-5-yl)pyridin-2-yl)cyclopropanecarboxamide (SU1714) (50 mg, 0.133 mmol, 1 Eq) in dry MeOH (3 mL) was added 10% Pd/C (15 mg, 0.14 mmol, 1 Eq). The reaction mixture was allowed to stir under H₂ atmosphere at room temperature overnight.

The reaction was then diluted with MeOH, filtered through celite, concentrated under reduced pressure and the crude residue was purified by flash column chromatography (0% to 10% MeOH in EtOAc) followed by HPLC purification to afford the target compound (34.3 mg, 0.09 mmol, 68%). ¹H NMR (500 MHz, DMSO-d₆) δ 11.57 (s, 1H), 10.79 (s, 1H), 8.42 (d, J=1.7 Hz, 1H), 8.32 (d, J=5.2 Hz, 1H), 7.98 (d, J=1.6 Hz, 1H), 7.42-7.32 (m, 2H), 5.50 (s, 2H), 4.27 (s, 1H), 2.91-2.83 (m, 2H), 2.05 (td, J=7.7, 3.9 Hz, 1H), 1.83-1.74 (m, 2H), 1.20 (s, 6H), 0.89-0.80 (m, 4H) ppm. LRMS: Calculated for C₂₁H₂₅N₅O₂ requires 379.46 found 380.2 (M+H).

### Example 293

### Methyl (4-(3-amino-7-(3,3-dimethylbutyl)-1H-indazol-5-yl)pyridin-2-yl)carbamate

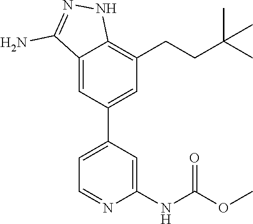 [SMILES: COC(=O)Nc1cc(-c2cc(CCC(C)(C)C)c3[nH]nc(N)c3c2)ccn1]

To a solution of methyl (4-(3-amino-7-(3,3-dimethylbut-1-yn-1-yl)-1H-indazol-5-yl)pyridin-2-yl)carbamate (SU1680) (100 mg, 0.254 mmol, 1 Eq) in dry MeOH (3 mL) was added 10% Pd/C (53.21 mg, 0.05 mmol, 0.2 Eq). The reaction mixture was allowed to stir under H₂ atmosphere at 40° C. overnight.

The reaction was then diluted with MeOH, filtered through celite, concentrated under reduced pressure and the crude residue was purified by flash column chromatography (0% to 10% MeOH in EtOAc) followed by HPLC purification to afford the target compound as a pale orange solid (83 mg, 0.226 mmol, 89%). ¹H NMR (500 MHz, DMSO-d₆) δ 11.63 (s, 1H), 10.16 (s, 1H), 8.27 (d, J=5.3 Hz, 1H), 8.14 (d, J=1.7 Hz, 1H), 7.97 (d, J=1.7 Hz, 1H), 7.36 (d, J=1.6 Hz, 1H), 7.33 (dd, J=5.3, 1.7 Hz, 1H), 5.49 (s, 2H), 3.70 (s, 3H), 2.82-2.75 (m, 2H), 1.62-1.56 (m, 2H), 1.00 (s, 9H) ppm. LRMS: Calculated for C₂₀H₂₅N₅O₂ requires 367.45 found 368.3 (M+H).

### Example 302

### 7-(3,3-dimethylbut-1-yn-1-yl)-5-(2-((1-methyl-1H-pyrazol-3-yl)amino)pyridin-4-yl)-1H-indazol-3-amine

### 4-Chloro-N-(1-methyl-1H-pyrazol-3-yl)pyridin-2-amine

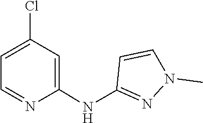

A mixture of palladium(II) acetate (30 mg, 0.12 mmol, 0.05 Eq) and xantphos (145 mg, 0.25 mmol, 0.1 Eq) in anhydrous toluene (2 mL) was stirred under nitrogen atmosphere for 15 min. The resulting mixture was added to a mixture of 2-bromo-4-chloropyridine (750 mg, 4 mmol, 1.5 Eq), 1-methyl-1H-pyrazol-3-amine (250 mg, 2.5 mmol, 1 Eq) and Cs₂CO₃ (1.6 g, 5 mmol, 2 Eq) in toluene (4 mL) under nitrogen atmosphere. The reaction mixture was allowed to stir at 110° C. for 30 h. The organic solvent was removed under reduced pressure. The crude residue was stirred in MeOH (10 mL) for 30 min., filtered and washed with MeOH (2×3 mL). The filtrate was concentrated under reduced pressure. The crude residue was purified by column chromatography (50% EtOAc in petroleum ether) to give the tilted product as white solid (210 mg, 1 mmol, 40%). ¹H NMR (500 MHz, DMSO) δ 9.50 (s, 1H), 8.09 (d, J=5.5 Hz, 1H), 7.53 (d, J=2.3 Hz, 1H), 7.41 (d, J=2.0 Hz, 1H), 6.78 (dd, J=5.5, 2.0 Hz, 1H), 6.24 (d, J=2.3 Hz, 1H), 3.76 (s, 3H). LC-MS: For C9H9ClN4 requires 208.65 found 209.1 (M+H).

### 7-(3,3-Dimethylbut-1-yn-1-yl)-5-(2-((1-methyl-1H-pyrazol-3-yl)amino)pyridin-4-yl)-1H-indazol-3-amine

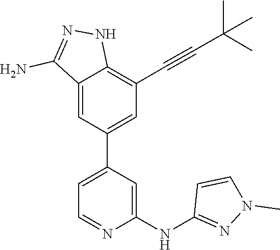 [SMILES: Cn1ccc(Nc2cc(-c3cc(C#CC(C)(C)C)c4[nH]nc(N)c4c3)ccn2)n1]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), 4-chloro-N-(1-methyl-1H-pyrazol-3-yl)pyridin-2-amine (0.31 g, 1.47 mmol, 1 Eq), cesium carbonate (1 g, 2.94 mmol, 2 Eq) and 1,1′-bis(di-tert-butylphosphino) ferrocene palladium chloride (144 mg, 0.22 mmol, 0.15 Eq) in dioxane (4 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure.

The crude residue was purified using column chromatography (10% MeOH in EtOAc) followed by trituration with MeOH (×2) then MeCN (×2) to give the titled product as white solid (113 mg, 0.3 mmol, 20%). ¹H NMR (500 MHz, DMSO) δ 11.82 (s, 1H), 9.18 (s, 1H), 8.15 (d, J=5.3 Hz, 1H), 8.11 (d, J=1.6 Hz, 1H), 7.52 (d, J=2.2 Hz, 2H), 7.48 (d, J=1.7 Hz, 1H), 7.00 (dd, J=5.3, 1.7 Hz, 1H), 6.39 (d, J=2.2 Hz, 1H), 5.62 (s, 2H), 3.77 (s, 3H), 1.38 (s, 9H). LRMS: For C22H23N7 requires 385.48 found 386.3 (M+H).

### Example 303

### 7-(3,3-dimethylbut-1-yn-1-yl)-5-(2-((1-methyl-1H-pyrazol-4-yl)amino)pyridin-4-yl)-1H-indazol-3-amine

### 4-chloro-N-(1-methyl-1H-pyrazol-4-yl)pyridin-2-amine

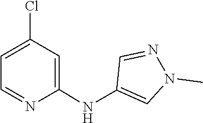

A mixture of palladium(II) acetate (30 mg, 0.12 mmol, 0.05 Eq) and xantphos (145 mg, 0.25 mmol, 0.1 Eq) in anhydrous toluene (2 mL) was stirred under nitrogen atmosphere for 15 min. The resulting mixture was added to a mixture of 2-bromo-4-chloropyridine (750 mg, 4 mmol, 1.5 Eq), 1-methyl-1H-pyrazol-4-amine (250 mg, 2.5 mmol, 1 Eq) and Cs₂CO₃ (1.6 g, 5 mmol, 2 Eq) in toluene (4 mL) under nitrogen atmosphere. The reaction mixture was allowed to stir at 110° C. for 30 h. The organic solvent was removed under reduced pressure. The crude residue was stirred in MeOH (10 mL) for 30 min., filtered and washed with MeOH (2×3 mL). The filtrate was concentrated under reduced pressure. The crude residue was purified by column chromatography (70% EtOAc in petroleum ether) to give the tilted product as white solid (140 mg, 0.7 mmol, 27%). ¹H NMR (500 MHz, DMSO) δ 8.98 (s, 1H), 8.07 (d, J=5.4 Hz, 1H), 7.94 (s, 1H), 7.44 (s, 1H), 6.72-6.65 (m, 2H), 3.81 (s, 3H). LC-MS: For C9H9ClN4 requires 208.65 found 209.2 (M+H).

### 7-(3,3-dimethylbut-1-yn-1-yl)-5-(2-((1-methyl-1H-pyrazol-4-yl)amino)pyridin-4-yl)-1H-indazol-3-amine

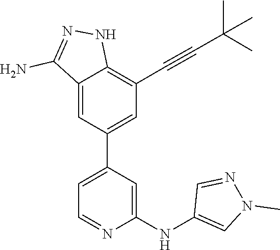 [SMILES: Cn1cc(Nc2cc(-c3cc(C#CC(C)(C)C)c4[nH]nc(N)c4c3)ccn2)cn1]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), 4-chloro-N-(1-methyl-1H-pyrazol-4-yl)pyridin-2-amine (0.31 g, 1.47 mmol, 1 Eq), cesium carbonate (1 g, 2.94 mmol, 2 Eq) and 1,1′-bis(di-tert-butylphosphino) ferrocene palladium chloride (144 mg, 0.22 mmol, 0.15 Eq) in dioxane (4 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (20% MeOH in EtOAc) followed by trituration with Et₂O (×5) to give the titled product as white solid (170 mg, 0.44 mmol, 30%). ¹H NMR (500 MHz, DMSO) δ 11.80 (s, 1H), 8.77 (s, 1H), 8.14 (d, J=5.3 Hz, 1H), 8.09 (d, J=1.7 Hz, 1H), 7.97 (s, 1H), 7.50 (d, J=1.7 Hz, 1H), 7.43 (s, 1H), 6.96-6.90 (m, 2H), 5.60 (s, 2H), 3.82 (s, 3H), 1.38 (s, 9H). LRMS: For C₂₂H₂₃N₇ requires 385.48 found 386.3 (M+H).

### Examples 304 and 305

### Methyl (4-(3-amino-7-((1-hydroxycyclopentyl)ethynyl)-1H-indazol-5-yl)pyridin-2-yl)carbamate (Example 304)

### 1-((3-amino-5-(2-(oxetan-3-ylamino)pyridin-4-yl)-1H-indazol-7-yl)ethynyl)cyclopentan-1-ol (Example 305)

### 4-(3-Amino-5-(4,4,5,5-tetramethyl-1,3,2-dioxaborolan-2-yl)-1H-indazol-7-yl)ethynyl)cyclopentan-1-ol

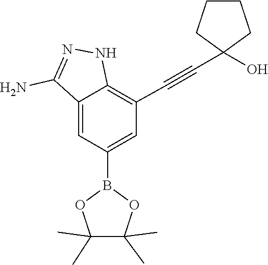 [SMILES: CC1(C)OB(c2cc(C#CC3(O)CCCC3)c3[nH]nc(N)c3c2)OC1(C)C]

A solution of 7-bromo-5-(4,4,5,5-tetramethyl-1,3,2-dioxaborolan-2-yl)-1H-indazol-3-amine (338 mg, 1 mmol, 1 Eq), 1-ethynylcyclopentan-1-ol (330 mg, 2 mmol, 3 Eq), copper (1) iodide (38 mg, 0.2 mmol, 0.2 Eq) and (1,1′-bis(diphenylphosphino)ferrocene)palladium(II) dichloride (73 mg, 0.1 mmol, 0.1 Eq) in 6 ml of N,N-dimethyformamide-triethylamine (4:1) was degassed in sealed tube. The reaction mixture was heated to 80° C. for 1 h the concentrated in vacuo. Flash column chromatography [petrol:ethyl acetate 1:0→0:1] afforded the title compound as a brown foam (240 mg, 0.65 mmol, 65%). ¹H NMR (400 MHz, DMSO-d₆) δ 11.75 (br s, 1H, NH), 8.15 (s, 1H, Ar—H), 7.49 (s, 1H, Ar—H), 5.62 (br s, 2H, NH₂), 5.23 (s, 1H, OH), 1.96-1.64 (m, 8H, ° Pent), 1.30 (s, 12H, BPin). LRMS: Calculated for C₂₀H₂₆BN₃O₃ requires 367.26 found 286.3 (M+1-C₆H₁₀).

### Methyl (4-(3-amino-7-((1-hydroxycyclopentyl)ethynyl)-1H-indazol-5-yl)pyridin-2-yl)carbamate (Example 304)

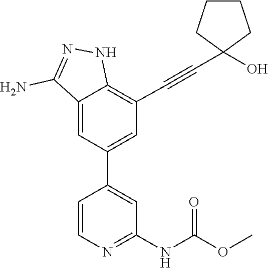 [SMILES: COC(=O)Nc1cc(-c2cc(C#CC3(O)CCCC3)c3[nH]nc(N)c3c2)ccn1]

To a microwave vial was added 4-(3-amino-5-(4,4,5,5-tetramethyl-1,3,2-dioxaborolan-2-yl)-1H-indazol-7-yl)ethynyl)cyclopentan-1-01 (125 mg, 0.340 mmol), methyl (4-iodopyridin-2-yl)carbamate (95 mg, 0.340 mmol), Pd(dtbpf)Cl₂ (25 mg, 0.034 mmol), and caesium carbonate (222 mg, 0.681 mmol) under an argon atmosphere. To the vial was added dioxane:water (9:1) and the mixture was degassed then stirred at 80° C. for 18 hours. The mixture was cooled to room temperature, diluted with ethyl acetate and saturated sodium carbonate solution, and the organic phase separated. The aqueous phase was extracted with ethyl acetate three times, and the combined organic phase was dried (magnesium sulfate), filtered, and concentrated in vacuo. Flash column chromatography [petrol:ethyl acetate:methanol 1:0:0→0:1:0→0:6:4] followed by flash column chromatography using basic silica [petrol:ethyl acetate:methanol 1:0:0→0:1:0→0:5:5] afforded the title compound as a dark yellow solid (16 mg, 12%). ¹H NMR (400 MHz, DMSO-d₆) δ 11.84 (br s, 1H, NH), 10.23 (br s, 1H, NH), 8.29 (d, J=5.2 Hz, 1H, Ar—H), 8.19 (s, 1H, Ar—H), 8.15 (s, 1H, Ar—H), 7.58 (s, 1H, Ar—H), 7.36 (d, J=5.2 Hz, 1H, Ar—H), 5.67 (br s, 2H, NH₂), 5.32 (s, 1H, OH), 3.71 (s, 3H, CH₃), 2.09-1.88 (m, 4H, ° Pent), 1.82-1.67 (m, 4H, ° Pent). LRMS: Calculated for C₂₁H₂₁N₅O₃ 391.16 found 392.3 (M+1).

### 1-((3-Amino-5-(2-(oxetan-3-ylamino)pyridin-4-yl)-1H-indazol-7-yl)ethynyl)cyclopentan-1-ol (Example 305)

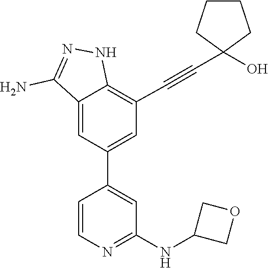 [SMILES: Nc1n[nH]c2c(C#CC3(O)CCCC3)cc(-c3ccnc(NC4COC4)c3)cc12]

To a microwave vial was added 4-(3-amino-5-(4,4,5,5-tetramethyl-1,3,2-dioxaborolan-2-yl)-1H-indazol-7-yl)ethynyl)cyclopentan-1-01 (125 mg, 0.340 mmol), 4-iodo-N-(oxetan-3-yl)pyridin-2-amine (94 mg, 0.340 mmol), Pd(dtbpf)Cl₂ (25 mg, 0.034 mmol), and caesium carbonate (222 mg, 0.681 mmol) under an argon atmosphere. To the vial was added dioxane:water (9:1) and the mixture was degassed then stirred at 80° C. for 18 hours. The mixture was cooled to room temperature, diluted with ethyl acetate and saturated sodium carbonate solution, and the organic phase separated. The aqueous phase was extracted with ethyl acetate three times, and the combined organic phase was dried (magnesium sulfate), filtered, and concentrated in vacuo. Flash column chromatography [petrol:ethyl acetate:methanol 1:0:0→0:1:0→0:6:4] followed by trituration from diethyl ether and methanol afforded the title compound as a dark yellow solid (9 mg, 7%). ¹H NMR (400 MHz, DMSO-d₆) δ 11.78 (br s, 1H, NH), 8.10 (s, 1H, Ar—H), 8.00 (d, J=5.1 Hz, 1H, Ar—H), 7.53 (s, 1H, Ar—H), 7.23 (d, J=5.1 Hz, 1H, Ar—H), 6.85 (d, J=5.1 Hz, 1H, Ar—H), 6.74 (br s, 1H, NH), 5.61 (br s, 2H, NH₂), 5.29 (s, 1H, OH), 5.03-4.89 (m, 1H, CH), 4.83 (t, J=6.0 Hz, 2H, CH₂), 4.46 (t, J=6.0 Hz, 2H, CH₂), 2.11-1.87 (m, 4H, ° Pent), 1.86-1.64 (m, 4H, ° Pent). LRMS: Calculated for C₂₂H₂₃N₅O₂ 389.46 found 390.3 (M+1).

### Section 9—Compounds of the Formula

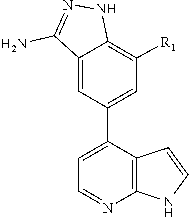

### Example 199

### 7-(Furan-3-yl)-5-(1H-pyrrolo[2,3-b]pyridin-4-yl)-1H-indazol-3-amine

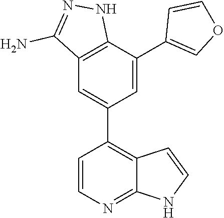 [SMILES: Nc1n[nH]c2c(-c3ccoc3)cc(-c3ccnc4[nH]ccc34)cc12]

A 2-5 mL MW tube was charged with a mixture of 7-chloro-5-(1H-pyrrolo[2,3-b]pyridin-4-yl)-1H-indazol-3-amine (Example 4) (50 mg, 0.15 mmol, 1 eq.), furan-3-ylboronic acid (98 mg, 0.3 mmol, 2 eq.), 1,1′-bis(diphenylphosphino)ferrocene]dichloropalladium(II) catalyst (16 mg, 0.0075 mmol, 5 mol %) in dioxane (oxygen-free, 1.4 mL) was heated to 50° C. under a gentle flow of nitrogen for 10 minutes, then 2 M aq. K₂CO₃ (oxygen-free, 0.6 mL, 0.9 mmol, 6 eq.) was added and the reaction mixture was heated to 110° C. for 16 hours. The reaction mixture was then cooled to room temperature, diluted water (10 mL), the mixture was extracted with EtOAc (3×20 mL) and the organic fractions combined and dried over magnesium sulfate, filtered and the solvent removed under reduced pressure. The resulting residue was purified by flash chromatography (Silica, 50 g, 1:1 pet ether: AcOEt to 100% EtOAc) to give the title compound as a pale yellow solid (33 mg, 70%). ¹H NMR (400 MHz, DMSO-D₆) δ 5.65 (s, 2H), 6.74 (dd, J=3.5, 1.8 Hz, 1H), 7.19 (d, J=1.5 Hz, 1H), 7.28 (d, J=1.4 Hz, 1H), 7.54-7.55 (m, 1H), 7.84 ((dd, J=3.5, 1.8 Hz, 1H), 8.15 (d, J=1.5 Hz, 1H), 8.29 (d, J=4.9 Hz, 1H), 8.44 (d, J=1.8 Hz, 1H), 11.53 (s, 1H), 11.73 (s, 1H). LRMS: Calculated for C₁₈H₁₃N₅O 315.11; Found: 316.0

### Example 200

### 7-Ethynyl-5-(1H-pyrrolo[2,3-b]pyridin-4-yl)-1H-indazol-3-amine

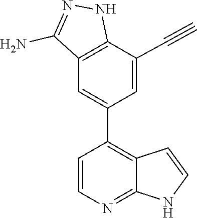 [SMILES: C#Cc1cc(-c2ccnc3[nH]ccc23)cc2c(N)n[nH]c12]

A 2-5 mL MW tube was charged with a mixture of 5-(1H-pyrrolo[2,3-b]pyridin-4-yl)-7-((triisopropylsilyl)ethynyl)-1H-indazol-3-amine (300 mg, 0.7 mmol), and THF (5 mL) and placed under nitrogen, TBAF (3.8 mL, 1M in THF) was then added and the solution allowed to stir for 20 min at room temperature. The reaction was quenched with the addition of water (5 mL), the mixture was extracted with EtOAc (3×10 mL) and the organic fractions combined and dried over magnesium sulfate, filtered and the solvent removed under reduced pressure. The resulting residue was purified by flash chromatography (Silica, 50 g, 1:1 pet ether: AcOEt to 100% EtOAc) to give the desired product as a pale yellow solid (132 mg, 69%). ¹H NMR (400 MHz, DMSO-D₆) δ 4.54 (s, 1H), 5.66 (s, 2H), 6.69 (dd, J=3.5, 1.8 Hz, 1H), 7.20 (d, J=1.5 Hz, 1H), 7.54-7.55 (m, 1H), 7.75 ((d, J=1.8 Hz, 1H), 8.26 (m, 2H), 11.76 (s, 1H), 11.96 (s, 1H). LRMS: Calculated for C₁₆H₁₁N₅ 273.1; Found: 274.1

### Example 201

### 7-(3,3-Dimethylbut-1-yn-1-yl)-5-(1H-pyrrolo[2,3-b]pyridin-4-yl)-1H-indazol-3-amine

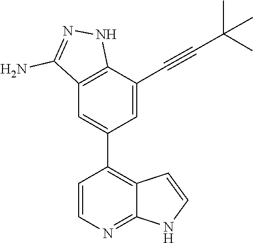 [SMILES: CC(C)(C)C#Cc1cc(-c2ccnc3[nH]ccc23)cc2c(N)n[nH]c12]

A 2-5 mL MW tube was charged with a mixture of 7-chloro-5-(1H-pyrrolo[2,3-b]pyridin-4-yl)-1H-indazol-3-amine (Example 4) (43 mg, 0.15 mmol, 1 eq.), (3,3-dimethylbut-1-yn-1-yl)trifluoroborate (56 mg, 0.3 mmol, 2 eq.), XphosG2 (5.9 mg, 0.0075 mmol, 5 mol %) in dioxane (oxygen-free, 1.4 mL) was heated to 50° C. under a gentle flow of nitrogen for 10 minutes, then 2 M aq. K₂CO₃ (oxygen-free, 0.45 mL, 0.9 mmol, 6 eq.) was added and the reaction mixture was heated to 110° C. for 11 hours. The reaction mixture was then cooled to room temperature, diluted water (10 mL), and filtered. The filtered solid was washed with water (10 mL×3), and purified by flash chromatography (Silica, 50 g, 0-2% MeOH in AcOEt) to give the title compound as a light beige solid (33 mg, 0.100 mmol, 67%). ¹H NMR (400 MHz, DMSO-D₆) δ 1.37 (s, 9H), 5.63 (s, 2H), 6.68 (dd, J=3.5, 1.8 Hz, 1H), 7.19 (d, J=4.9 Hz, 1H), 7.52-7.55 (m, 1H), 7.57 (d, J=1.5 Hz, 1H), 8.17 (d, J=1.5 Hz, 1H), 8.25 (d, J=4.9 Hz, 1H), 11.74 (s, 1H), 11.80 (s, 1H). HRMS: Calculated for C₂₀H₁₉N₅ 329.1640;

### Example 202

### 7-(Cyclopropylethynyl)-5-(1H-pyrrolo[2,3-b]pyridin-4-yl)-1H-indazol-3-amine

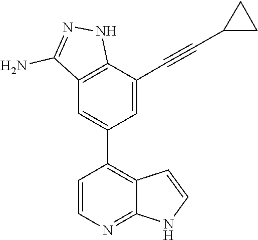 [SMILES: Nc1n[nH]c2c(C#CC3CC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), ethynylcyclopropane (30 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (11 mg, 26%). ¹H NMR (400 MHz, DMSO-D₆) δ 0.91 (s, 4H), 1.62 (m, 1H), 5.60 (s, 2H), 6.68 (dd, J=3.5, 1.8 Hz, 1H), 7.18 (d, J=1.5 Hz, 1H), 7.54-7.55 (m, 1H), 7.61 (d, J=1.8 Hz, 1H), 8.15 (d, J=1.8 Hz, 1H), 8.25 (m, 2H), 11.73 (s, 1H), 11.82 (s, 1H). LRMS: Calculated for C₁₉H₁₅N₅ 313.13; Found: 314.1

### Example 203

### 7-(Cyclopentylethynyl)-5-(1H-pyrrolo[2,3-b]pyridin-4-yl)-1H-indazol-3-amine

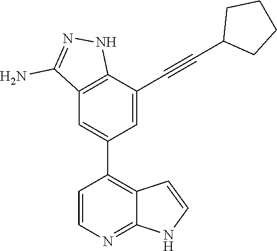 [SMILES: Nc1n[nH]c2c(C#CC3CCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), ethynylcyclopentane (32 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (19 mg, 40%). ¹H NMR (400 MHz, DMSO-D₆) δ 1.24 (s, 1H), 1.6 (m, 2H), 1.77 (m, 4H), 2.01 (m, 2H), 5.60 (s, 2H), 6.69 (dd, J=3.5, 1.8 Hz, 1H), 7.18 (d, J=1.5 Hz, 1H), 7.54-7.55 (m, 1H), 7.60 (d, J=1.8 Hz, 1H), 8.16 (d, J=1.8 Hz, 1H), 8.26 (m, 2H), 11.73 (s, 1H), 11.81 (s, 1H). LRMS: Calculated for C₂₁H₁₉N₅ 341.16; Found: 342.2

### Example 204

### 7-(Cyclohexylethynyl)-5-(1H-pyrrolo[2,3-b]pyridin-4-yl)-1H-indazol-3-amine

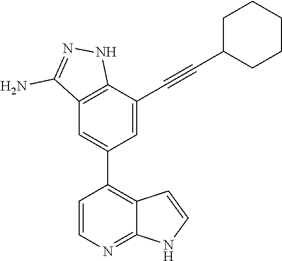 [SMILES: Nc1n[nH]c2c(C#CC3CCCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), ethynylcyclohexane (33 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (26 mg, 46%). Calculated for C₂₂H₂₁N₅ 355.18; Found: 356.0

### Example 205

### 3-(3-Amino-5-(1H-pyrrolo[2,3-b]pyridin-4-yl)-1H-indazol-7-yl)prop-2-yn-1-ol

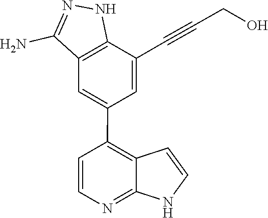 [SMILES: Nc1n[nH]c2c(C#CCO)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), prop-2-yn-1-ol (30 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (22 mg, 41%)¹H NMR (400 MHz, DMSO-D₆) δ 4.2 (s, 2H), 5.44 s, 1H), 5.58 (s, 2H), 6.67 (dd, J=3.5, 1.8 Hz, 1H), 7.18 (d, J=1.5 Hz, 1H), 7.52-7.54 (m, 1H), 7.61 (d, J=1.8 Hz, 1H), 8.16 (d, J=1.8 Hz, 1H), 8.26 (m, 2H), 11.73 (s, 1H), 11.81 (s, 1H). LRMS: Calculated for C₁₇H₁₃N₅O 303.11; Found: 304.1

### Example 206

### 4-(3-Amino-5-(1H-pyrrolo[2,3-b]pyridin-4-yl)-1H-indazol-7-yl)-2-methylbut-3-yn-2-ol

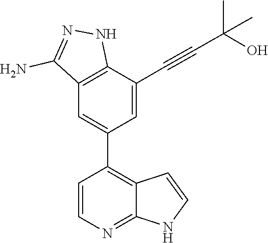 [SMILES: CC(C)(O)C#Cc1cc(-c2ccnc3[nH]ccc23)cc2c(N)n[nH]c12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 2-methylbut-3-yn-2-ol (30 uL, 0.3 mmol, 2 eq.), 1,1′-bis(diphenyl phosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (16 mg, 33%)¹H NMR (400 MHz, DMSO-D₆) δ 1.54 (s, 2H), 5.46 (s, 1H), 5.68 (s, 2H), 6.69 (dd, J=3.5, 1.8 Hz, 1H), 7.20 (d, J=1.5 Hz, 1H), 7.52-7.54 (m, 1H), 7.62 (d, J=1.8 Hz, 1H), 8.21 (d, J=1.8 Hz, 1H), 8.26 (m, 2H), 11.76 (s, 1H), 11.81 (s, 1H). LRMS: Calculated for C₁₉H₁₇N₅O 331.14; Found: 332.1.

### Example 207

### 1-((3-Amino-5-(1H-pyrrolo[2,3-b]pyridin-4-yl)-1H-indazol-7-yl)ethynyl)cyclopentan-1-ol

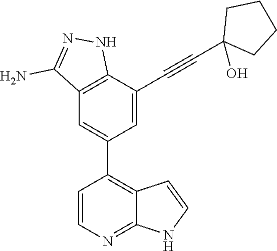 [SMILES: Nc1n[nH]c2c(C#CC3(O)CCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 1-ethynylcyclopentan-1-ol (30 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (22 mg, 41%)¹H NMR (400 MHz, DMSO-D₆) δ 1.24 (s, 1H), 1.6 (m, 2H), 1.77 (m, 4H), 2.01 (m, 2H), 5.44 (s, 1H), 5.60 (s, 2H), 6.69 (dd, J=3.5, 1.8 Hz, 1H), 7.18 (d, J=1.5 Hz, 1H), 7.54-7.55 (m, 1H), 7.60 (d, J=1.8 Hz, 1H), 8.16 (d, J=1.8 Hz, 1H), 8.26 (m, 2H), 11.73 (s, 1H), 11.81 (s, 1H). LRMS: Calculated for C₂₁H₁₉N₅O 357.16; Found: 358.1.

### Example 208

### 1-((3-Amino-5-(1H-pyrrolo[2,3-b]pyridin-4-yl)-1H-indazol-7-yl)ethynyl)cyclohexan-1-ol

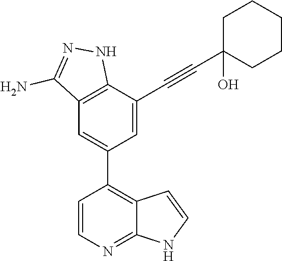 [SMILES: Nc1n[nH]c2c(C#CC3(O)CCCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 1-ethynylcyclohexan-1-ol (33 uL, 0.3 mmol, 2 eq.), 1,1′-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (26 mg, 46%). ¹H NMR (500 MHz, DMSO) δ 12.03 (s, 1H), 8.36-8.27 (m, 2H), 7.72 (d, J=1.6 Hz, 1H), 7.64 (t, J=2.9 Hz, 1H), 7.33 (d, J=5.2 Hz, 1H), 6.78 (dd, J=3.6, 1.7 Hz, 1H), 1.97 (dd, J=11.7, 6.5 Hz, 2H), 1.72-1.53 (m, 6H), 1.51-1.40 (m, 1H), 1.29 (d, J=13.9 Hz, 1H). 5 LRMS: Calculated for C₂₂H₂₁N₅O 371.17; Found: 372.2.

### Example 209

### 1-((3-Amino-5-(1H-pyrrolo[2,3-b]pyridin-4-yl)-1H-indazol-7-yl)ethynyl)cycloheptan-1-ol

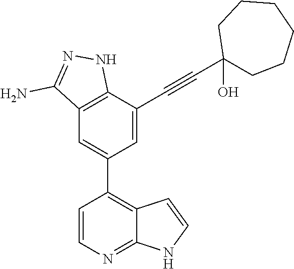 [SMILES: Nc1n[nH]c2c(C#CC3(O)CCCCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 1-ethynylcycloheptan-1-ol (35 uL, 0.3 mmol, 2 eq.), 1,1-bis(diphenylphosphino)ferrocene]dichloropalladium(II) catalyst (16 mg mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (26 mg, 46%). ¹H NMR (500 MHz, DMSO) δ 11.76 (s, 1H), 8.37-8.15 (m, 1H), 7.64 (s, 1H), 7.56 (t, J=3.1 Hz, 1H), 7.20 (d, J=5.0 Hz, 1H), 6.69 (d, J=2.8 Hz, 1H), 5.67 (s, 1H), 2.15-2.07 (m, 2H), 1.94-1.83 (m, 2H), 1.71-1.42 (m, 8H). LRMS: Calculated for C₂₃H₂₃N₅O 385.2; Found: 386.2.

### Example 210

### 7-(5-Morpholinopent-1-yn-1-yl)-5-(1H-pyrrolo[2,3-b]pyridin-4-yl)-1H-indazol-3-amine

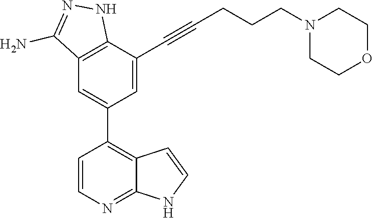 [SMILES: Nc1n[nH]c2c(C#CCCCN3CCOCC3)cc(-c3ccnc4[nH]ccc34)cc12]

A 0.5-2 mL MW tube was charged with a mixture of 7-(5-chloropent-1-yn-1-yl)-5-(1H-pyrrolo[2,3-b]pyridin-4-yl)-1H-indazol-3-amine (52 mg, 0.15 mol, 1 eq.) and morpholine (131 μL, 1.5 mmol, 10 eq.) in acetonitrile (1.4 mL) and sealed under nitrogen. The reaction mixture was heated to 100° C. for 6 hours and then cooled to room temperature and diluted with AcOEt (100 mL) and washed with 1 M aq. Na₂CO₃ (30 mL×3), dried (MgSO₄) and purified by flash chromatography (Silica, 50 g, 1-10%, MeOH in AcOEt containing 1% triethylamine) to give the title compound as a yellow solid (21 mg, 35%). ¹H NMR (400 MHz, DMSO) δ 1.77-1.87 (m, 2H), 2.35-2.47 (m, 6H), 2.53-2.57 (m, 2H), 3.58 (t, J=4.7 Hz, 4H), 5.59 (s, 2H), 6.68 (dd, J=3.5, 1.9 Hz, 1H), 7.18 (d, J=5.0 Hz, 1H), 7.53 (dd, J=3.5, 2.5 Hz, 1H), 7.62 (d, J=1.6 Hz, 1H), 8.16 (d, J=1.5 Hz, 1H), 8.25 (d, J=5.0 Hz, 1H), 11.72 (s, 1H), 11.82 (s, 1H).

### Example 211

### 7-(4-(Piperidin-1-yl)but-1-yn-1-yl)-5-(1H-pyrrolo[2,3-b]pyridin-4-yl)-1H-indazol-3-amine

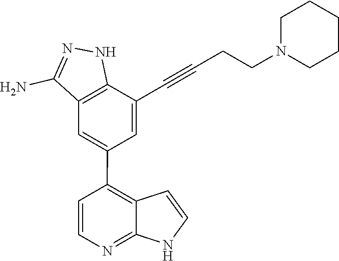 [SMILES: Nc1n[nH]c2c(C#CCCN3CCCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 1-(but-3-yn-1-yl)piperidine (33 uL, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (26 mg, 46%). ¹H NMR (400 MHz, DMSO-D₆) δ 1.41 (m, 2H), 1.53 (m, 4H), 2.24 (m, 4H), 2.66 (m, 4H), 5.62 (s, 2H), 6.68 (dd, J=3.5, 1.8 Hz, 1H), 7.18 (d, J=1.5 Hz, 1H), 7.19 (d, J=1.5 Hz, 1H), 7.53 (m, 1H), 7.61 (d, J=1.8 Hz, 1H), 8.17 (d, J=1.8 Hz, 1H), 8.26 (m, 2H), 11.74 (s, 1H), 11.83 (s, 1H). LRMS: Calculated for C₂₃H₂₄N₆ 384.21; Found: 385.2.

### Example 212

### 7-(5-(Piperidin-1-yl)pent-1-yn-1-yl)-5-(1H-pyrrolo[2,3-b]pyridin-4-yl)-1H-indazol-3-amine

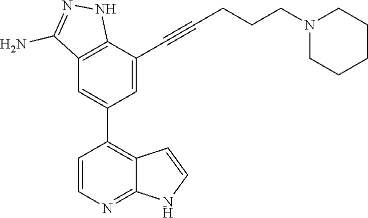 [SMILES: Nc1n[nH]c2c(C#CCCCN3CCCCC3)cc(-c3ccnc4[nH]ccc34)cc12]

A 2-5 mL MW tube was charged with a mixture of 7-(5-chloropent-1-yn-1-yl)-5-(1H-pyrrolo[2,3-b]pyridin-4-yl)-1H-indazol-3-amine (70 mg, 0.2 mL, 1 eq.) and piperidine (198 μL, 2 mmol, 10 eq.) in acetonitrile (1.8 mL) and sealed under nitrogen. The reaction mixture was heated to 100° C. for 6 hours and then cooled to room temperature and diluted with AcOEt (100 mL) and washed with 1 M aq. Na₂CO₃ (50 mL×3), dried (MgSO₄) and purified by flash chromatography (Silica, 50 g, 1-12%, MeOH in AcOEt containing 1% triethylamine) to give the title compound as a yellow solid (8 mg, 10%). ¹H NMR (400 MHz, DMSO-D₆) δ 1.33-1.42 (m, 2H), 1.44-1.55 (m, 4H), 1.74-1.83 (m, 2H), 2.54 (d, J=2.3 Hz, 4H), 5.62 (s, 2H), 6.68 (dd, J=3.5, 1.8 Hz, 1H), 7.18 (d, J=4.9 Hz, 1H), 7.50-7.57 (m, 1H), 7.62 (d, J=1.6 Hz, 1H), 8.16 (d, J=1.6 Hz, 1H), 8.25 (d, J=4.9 Hz, 1H), 11.75 (s, 1H), 11.84 (s, 1H). A CH₂ peak not observed due solvent peak.

### Example 213

### 7-(6-(Piperidin-1-yl)hex-1-yn-1-yl)-5-(1H-pyrrolo[2,3-b]pyridin-4-yl)-1H-indazol-3-amine

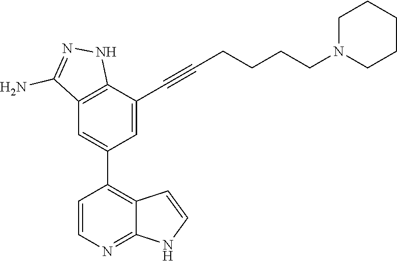 [SMILES: Nc1n[nH]c2c(C#CCCCCN3CCCCC3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), 1-(hex-5-yn-1-yl)piperidine (33 uL, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (14 mg, 20%). ¹H NMR (400 MHz, DMSO-D₆) δ 1.66-1.95 (m, 10H), 2.70 (m, 6H), 3.7 (m, 2H), 5.71 (s, 2H), 6.70 (dd, J=3.5, 1.8 Hz, 1H), 7.20 (d, J=1.5 Hz, 1H), 7.25 (d, J=1.5 Hz, 1H), 7.54 (m, 2H), 8.23 (d, J=1.8 Hz, 1H), 8.29 (m, 1H), 11.72 (s, 1H), 11.79 (s, 1H). LRMS: Calculated for C₂₅H₂₈N₆ 412.24; Found: 413.2.

### Example 214

### 6-(3-Amino-5-(1H-pyrrolo[2,3-b]pyridin-4-yl)-1H-indazol-7-yl)hex-5-ynoic acid

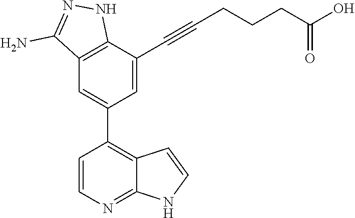 [SMILES: Nc1n[nH]c2c(C#CCCCC(=O)O)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), hex-5-ynoic acid (30 uL, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (22 mg, 40%). ¹H NMR (400 MHz, DMSO-D₆) δ 0.97 (t, J=7.0 Hz, 2H), 1.86 (q, J=7.3 Hz, 2H), 2.44 (t, J=7.3 Hz, 2H), 5.63 (s, 2H), 6.69 (dd, J=3.5, 1.8 Hz, 1H), 7.20 (d, J=1.5 Hz, 1H), 7.54 (d, J=1.5 Hz, 1H), 7.64 (d, J=1.5 Hz 2H), 8.18 (d, J=1.8 Hz, 1H), 8.26 (m, 1H), 11.75 (s, 1H), 11.79 (s, 1H). LRMS: Calculated for C₂₀H₁₇N₅O₂ 359.14; Found: 360.2.

### Example 215

### 7-(3-Amino-5-(1H-pyrrolo[2,3-b]pyridin-4-yl)-1H-indazol-7-yl)hept-6-ynoic acid

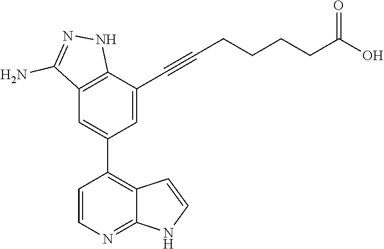 [SMILES: Nc1n[nH]c2c(C#CCCCCC(=O)O)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), hept-6-ynoic acid (31 uL, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (18 mg, 33%). ¹H NMR (400 MHz, DMSO-D₆) δ 1.67 (m, 6H), 2.28 (t, J=7.3 Hz, 2H), 5.63 (s, 2H), 6.69 (dd, J=3.5, 1.8 Hz, 1H), 7.18 (d, J=1.5 Hz, 1H), 7.54 (d, J=1.5 Hz, 1H), 7.62 (d, J=1.5 Hz 2H), 8.17 (d, J=1.8 Hz, 1H), 8.26 (m, 1H), 11.75 (s, 1H), 11.79 (s, 1H). LRMS: Calculated for C₂₁H₁₉N₅O₂ 373.15; Found: 374.2.

### Example 216

### 7-(4-Phenoxybut-1-yn-1-yl)-5-(1H-pyrrolo[2,3-b]pyridin-4-yl)-1H-indazol-3-amine

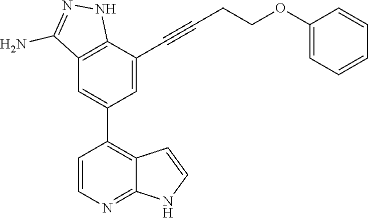 [SMILES: Nc1n[nH]c2c(C#CCCOc3ccccc3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), (but-3-yn-1-yloxy)benzene (52 mg, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (28 mg, 46%). ¹H NMR (400 MHz, DMSO-D₆) δ 3.01 (t, J=7.3 Hz 2H), 4.29 (t, J=7.3 Hz, 2H), 5.63 (s, 2H), 6.69 (dd, J=3.5, 1.8 Hz, 1H), 6.95 (t, J=7.2 Hz, 1H), 7.03 (m, 1H) 7.19 (d, J=1.5 Hz, 1H), 7.31 (m, 2H), 7.54 (t, J=2.8 Hz, 1H), 7.65 (d, J=1.5 Hz 2H), 8.20 (d, J=1.8 Hz, 1H), 8.26 (m, 1H), 11.74 (s, 1H), 11.89 (s, 1H). LRMS: Calculated for C₂₄H₁₉N₅O 393.16; Found: 394.1.

### Example 217

### 7-(6-Phenoxyhex-1-yn-1-yl)-5-(1H-pyrrolo[2,3-b]pyridin-4-yl)-1H-indazol-3-amine

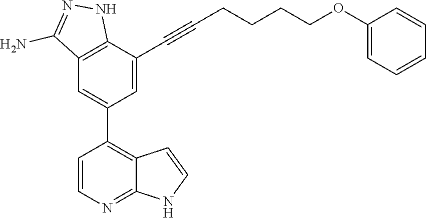 [SMILES: Nc1n[nH]c2c(C#CCCCCOc3ccccc3)cc(-c3ccnc4[nH]ccc34)cc12]

A 2-5 mL MW tube was charged with a mixture of 7-bromo-5-(1H-pyrrolo[2,3-b]pyridin-4-yl)-1H-indazol-3-amine (Example 5) (50 mg, 0.15 mmol, 1 eq.), (hex-5-yn-1-yloxy)benzene (52 mg, 0.3 mmol, 2 eq.), 1,1′-bis(diphenylphosphino)ferrocene]dichloropalladium(II) catalyst (16 mg, 0.02 mmol, 15 mol %) and Cul (4.2 mg 0.02 mmol, 15 mol %) in DMF (1 mL) and NEt₃ (1 mL). The tube was then heated to 85° C. under nitrogen for 16 hrs. The reaction mixture was then cooled to room temperature, diluted with water (10 mL), the mixture was extracted with EtOAc (3×10 mL) and the organic fractions combined and dried over magnesium sulfate, filtered and the solvent removed under high vacuum. The resulting residue was purified by HPLC to give the desired product as a yellow solid (22 mg, 33%). ¹H NMR (400 MHz, DMSO-D₆) δ 1,80 (m, 2H), 1,91 (m, 2H), 2.61 (t, J=7.3 Hz, 2H) 4.05 (t, J=7.3 Hz, 2H), 5.63 (s, 2H), 6.69 (dd, J=3.5, 1.8 Hz, 1H), 6.95 (m, 3H), 7.19 (d, J=1.5 Hz, 1H), 7.36 (m, 2H), 7.53 (t, J=2.8 Hz, 1H), 7.63 (d, J=1.5 Hz 2H), 8.17 (d, J=1.8 Hz, 1H), 8.26 (m, 1H), 11.75 (s, 1H), 11.87 (s, 1H). LRMS: Calculated for C₂₅H₂₁N₅0 407.17; Found: 408.2.

### Section 10—Compounds of the Formula

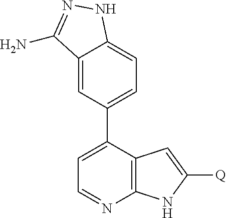

General procedure A:

A suspension of the required 4-chloroazaindole substrate (1 eq.), 5-(4,4,5,5-tetramethyl-1,3,2-dioxaborolan-2-yl)-1H-indazol-3-amine (1.2-1.5 eq.) and base (2 eq.) in 1:3 of solvent was deoxygenated with nitrogen in sealed tube. Then [1,1′-bis(di-tert-butylphosphino)ferrocene]dichloropalladium(II) catalyst (0.05 eq.) was added then the tube was sealed and the mixture allowed to stirred at 90-100° C. for 18 h. After the reaction was cooled to room temperature, EtOAc and water were added. Extracted organic layer was dried over magnesium sulfate and concentrated under reduced pressure and the residue purified by column chromatography.

### Example 218

### 4-Chloro-3-(cyclopropylethynyl)pyridin-2-amine

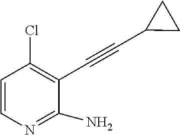 [SMILES: Nc1nccc(Cl)c1C#CC1CC1]

A solution of 4-chloro-3-iodo-pyridine-2-amine (0.12 g, 0.4 mmol), copper (1) iodide (0.004 g, 0.02 mmol) and bis(triphenylphosphine) palladium(II) chloride (0.014 g, 0.02 mmol) in 3 ml of N,N-dimethyformamide-triethylamine (1:4) was degassed in sealed tube followed by addition of ethynylcyclopropane (0.06 g, 0.7 mmol). The reaction mixture was stirred at 80° C. for 4 h. The reaction mixture was diluted with EtOAc and extracted with 1 M sodium carbonate. The extracted organic layer was washed with brine and dried over magnesium sulfate and concentrated under reduced pressure and the residue purified by column chromatography (25% EtOAc and 1% triethylamine in petroleum ether 60-80%) to give the product as yellow oil (0.8 g, 90%), ¹H NMR (400 MHz, DMSO-d₆) δ ppm 0.81-0.87 (m, 2H) 0.89-0.95 (m, 2H) 1.63 (tt, J=8.24, 5.04 Hz, 1H). 6.36 (br. s., 2H) 6.66 (d, J=5.49 Hz, 1H) 7.81 (d, J=5.34 Hz, 1H). ¹³C NMR (100 MHz, DMSO-d₆) δ ppm 0.50, 7.99, 67.38, 100.90, 103.76, 111.46, 143.07, 146.44, 146.43, 160.05. m/z (ESI-MS) [M]⁺ 193.1.

### 4-Chloro-2-cyclopropyl-7-azaindole

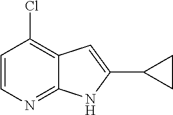 [SMILES: Clc1ccnc2[nH]c(C3CC3)cc12]

To a solution of 4-chloro-3-(cyclopropylethynyl)pyridin-2-amine (0.108 g, 0.56 mmol) in 1,4 dioxane (2 ml), potassium tert-butoxide (0.157 g, 1.4 mmol) and 18-crown-6 (0.014 g, 10%, 0.056 mmol) were added and the mixture was stirred at 110° C. for 18 h. After the reaction was cooled to room temperature, EtOAc and 1 M sodium carbonate solution were added. The extracted organic layer was dried over magnesium sulfate and concentrated under reduced pressure to give the product as yellow solid (96 mg, 88%). ¹H NMR (400 MHz, DMSO-d₆) b ppm 0.82-0.91 (m, 2H) 0.97-1.05 (m, 2H) 2.05 (tt, J=8.40, 5.22 Hz, 1H) 6.15 (s, 1H) 7.09 (d, J=5.27 Hz, 1H) 8.02 (d, J=4.83 Hz, 1H) 11.87 (br. s., 1H). ¹³C NMR (100 MHz, DMSO-d₆) b ppm 9.24, 9.85, 70.20, 93.15, 115.73, 120.08, 132.48, 142.10, 145.62, 149.71. m/z (ESI-MS) [M]⁺ 193.0.

### 5-(2-Cyclopropyl-1H-pyrrolo[2,3-b]pyridin-4-yl)-1H-indazol-3-amine

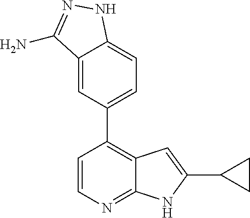 [SMILES: Nc1n[nH]c2ccc(-c3ccnc4[nH]c(C5CC5)cc34)cc12]

4-Chloro-2-cyclopropyl-7-azaindole (0.074 g, 0.39 mmol), 5-(4,4,5,5-tetramethyl-1,3,2-dioxaborolan-2-yl)-1H-indazol-3-amine (0.15 g, 0.58 mmol), 1M potassium phosphate solution (0.78 ml, 0.78 mmol), [1,1′-bis(di-tert-butylphosphino)ferrocene]dichloropalladium(II) catalyst (0.012 g, 0.195 mmol) in 2.3 ml ethanol were reacted as described in General procedure A and chromatographic purification (60% EtOAc and 1% triethylamine in petroleum ether 60-80%), gave the titled compound as white solid (84 mg, 74%). ¹H NMR (400 MHz, DMSO-d₆) δ ppm 0.85-0.91 (m, 2H) 0.95-1.04 (m, 2H) 2.07 (tt, J=8.35, 5.27 Hz, 1H) 5.52 (s, 2H) 6.38 (d, J=1.76 Hz, 1H) 7.10 (d, J=4.83 Hz, 1H) 7.36 (d, J=8.35 Hz, 1H) 7.63 (dd, J=8.57, 1.54 Hz, 1H) 8.10-8.14 (m, 2H) 11.52 (m, 2H). ¹³C NMR (100 MHz, DMSO-d₆) δ ppm 10.01, 14.66, 21.33, 95.04, 110.34, 114.50, 115.11, 118.70, 120.64, 127.18, 128.68, 140.04, 141.61, 141.93, 143.98, 149.97, 150.37. m/z (ESI-HRMS) calculated for C₁₉H₂₁N₆=333.1822 found=333.1820.

### Example 219

### 2-Cyclohexyl-7-azaindole

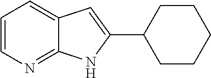 [SMILES: c1cnc2[nH]c(C3CCCCC3)cc2c1]

To a stirred solution of 2(Boc-amino)-3-methyl-pyridine (1.04 g, 4.7 mmol) in anhydrous THF (20 ml) at −4° C., 2M n-butyllithium (5.8 ml, 11.7 mmol) was added slowly over 15 min, then 2-cyclohexyl-N-methoxy-N-methylcarboxamide (0.9 g, 5.2 mmol) was added slowly and the reaction mixture allowed to stirred for 2 h. Thereafter, hydrochloric acid (5M, 50 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated. The crude then purified using column chromatography (15% EtOAc in petroleum ether 60-80%) to give the product as white solid (188 mg, 18%) 1H NMR (400 MHz, DMSO-d₆) δ ppm 1.12-1.56 (m, 6H) 1.70-2.02 (m, 4H) 2.59-2.79 (m, 1H) 6.11 (d, J=1.32 Hz, 1H) 6.96 (dd, J=7.91, 4.83 Hz, 1H) 7.72-7.83 (m, 1H) 8.09 (dd, J=4.61, 1.54 Hz, 1H) 11.45 (br. s., 1H). ¹³C NMR (100 MHz, DMSO-d₆) b ppm 26.20, 26.36, 32.65, 37.43, 95.11, 115.69, 120.88, 127.30, 141.66, 146.86, 149.20. m/z (ESI-MS) [M]⁺ 201.1.

### 2-Cyclohexyl-7-oxide-7-azaindole

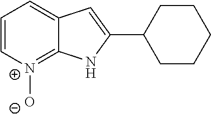 [SMILES: [O-][n+]1cccc2cc(C3CCCCC3)[nH]c21]

To an ice cooled solution of 2-cyclohexyl-7-azaindole (0.18 g, 0.9 mmol) in EtOAc, meta-chloroperoxybenzoic acid (0.25 g, 1.4 mmol) was added slowly then the reaction warmed to room temperature and stirred to 1 h. After the reaction was completed, the solvent was evaporated, treated with 1M sodium carbonate solution and extracted with EtOAc. The residue then concentrated under vacuum to give the product as a yellow solid (0.15 g, 83%), ¹H NMR (400 MHz, DMSO-d₆) δ ppm 1.13-1.40 (m, 6H) 1.59-1.62 (m, 4H) 2.60 (m, 1H) 6.27 (s, 1H) 7.05 (dd, J=7.91, 6.15 Hz, 1H) 7.50 (d, J=7.91 Hz, 1H) 8.04 (d, J=6.15 Hz, 1H) 11.90 (br. s., 1H). m/z (ESI-MS) [M]⁺ 217.

### 2-Cyclohexyl-4-chloro-7-azaindole

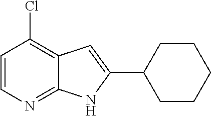 [SMILES: Clc1ccnc2[nH]c(C3CCCCC3)cc12]

In sealed tube, 2-cyclohexyl-7-oxide-7-azaindole (0.15 g, 0.7 mmol) was dissolved in phosphoryl chloride (0.2 ml, 2 mmol) and the reaction heated to 90° C. and stirred to 18 h. The reaction mixture then warmed to room temperature and poured in to water and neutralized with saturated sodium carbonate solution and extract with EtOAc. The collected organic layer dried over magnesium sulfate, concentrated and purified using column chromatography (30% EtOAc in petroleum ether 60-80%) to afford the titled compound as white solid (92 mg, 60%). ¹H NMR (400 MHz, DMSO-d₆) δ ppm 1.07-1.31 (m, 6H) 1.68-2.03 (m, 4H) 2.66-2.82 (m, 1H) 6.18 (d, J=1.32 Hz, 1H) 7.11 (d, J=5.27 Hz, 1H) 8.06 (d, J=5.27 Hz, 1H) 11.89 (br. s., 1H). ¹³C NMR (100 MHz, DMSO-d₆) δ ppm 26.12, 26.30, 32.49, 37.39, 93.39, 115.60, 119.72, 131.21.

### 5-(2-Cyclohexyl-1H-pyrrolo[2,3-b]pyridin-4-yl)-1H-indazol-3-amine

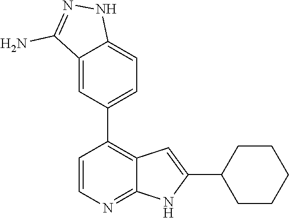 [SMILES: Nc1n[nH]c2ccc(-c3ccnc4[nH]c(C5CCCCC5)cc34)cc12]

2-Cyclohexyl-4-chloro-7-azaindole (0.023 g, 0.1 mmol), 5-(4,4,5,5-tetramethyl-1,3,2-dioxaborolan-2-yl)-1H-indazol-3-amine (0.04 g, 0.15 mmol), 1M potassium phosphate solution (0.2 ml, 0.2 mmol), [1,1′-bis(di-tert-butylphosphino)ferrocene]dichloropalladium(II) catalyst (0.0012 g, 0.005 mmol) in 0.6 ml ethanol were reacted as described in General procedure A and chromatographic purification (70% EtOAc in petroleum ether 60-80%), gave the titled compound as yellow solid (13.6 mg, 41%). ¹H NMR (400 MHz, DMSO-d₆) δ ppm 1.18-1.38 (m, 4H) 1.52-1.54 (m, 2H) 1.71-1.81 (m, 4H) 2.68-2.81 (m, 1H) 5.52 (s, 2H) 6.39 (d, J=1.76 Hz, 1H) 7.10 (d, J=5.27 Hz, 1H) 7.37 (d, J=8.79 Hz, 1H) 7.64 (dd, J=8.57, 1.54 Hz, 1H) 8.08-8.17 (m, 2H) 11.54 (m, 2H). ¹³C NMR (100 MHz, DMSO-d₆) 26.21, 26.42, 32.69, 37.58, 94.80, 110.36, 114.39, 115.12, 118.36, 120.65, 127.23, 128.73, 140.64, 141.63, 142.18, 146.92, 149.95, 150.36. m/z (ESI-HRMS) calculated for C₂₀H₂₂N₅₌332.1870 found=332.1867.

### Example 220

### 2-Neopentyl-7-azaindole

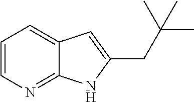

To a stirred solution of 2(Boc-amino)-3-methyl-pyridine (2.3 g, 10.5 mmol) in anhydrous THF (50 ml) at −4° C., 2M n-butyllithium (13.1 ml, 26.25 mmol) was added slowly over 15 min, then N-methoxy-N,3,3-trimethylbutanamide (2 g, 12.5 mmol) was added slowly and the reaction mixture stirred for 2 h. Thereafter, hydrochloric acid (5M, 50 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated. The crude then purified using column chromatography (15% EtOAc in petroleum ether 60-80%) to give the product as white solid (1 g, 50%), ¹H NMR (400 MHz, DMSO-d₆) δ ppm 0.94 (s, 9H) 2.59 (s, 2H) 6.11 (d, J=1.76 Hz, 1H) 6.97 (dd, J=7.91, 4.83 Hz, 1H) 7.80 (dd, J=7.69, 1.54 Hz, 1H) 8.10 (dd, J=4.83, 1.76 Hz, 1H) 11.41 (br. s., 1H). ¹³C NMR (100 MHz, DMSO-d₆) δ ppm 29.80, 31.94, 42.38, 99.13, 115.63, 121.04, 127.04, 139.10, 141.51, 148.80. m/z (ESI-MS) [M]⁺ 189.1.

### 2-Neopentyl-7-oxide-7-azaindole

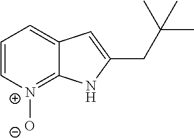

To an ice-cooled solution of 2-neopentyl-7-azaindole (0.85 g, 4.5 mmol) in EtOAc, meta-chloroperoxybenzoic acid (1.2 g, 7.2 mmol) was added slowly then the reaction warmed to room temperature and stirred to 1 h. After the reaction was completed, the solvent was evaporated, treated with 1M sodium carbonate solution and extracted with EtOAc. The residue then concentrated under vacuum to give the product as a yellow solid (0.95 g, 99%). ¹H NMR (400 MHz, DMSO-d₆) δ ppm 0.91 (s, 9H) 2.63 (s, 2H) 6.27 (s, 1H) 7.02 (dd, J=8.13, 6.37 Hz, 1H) 7.54 (d, J=7.91 Hz, 1H) 8.05 (d, J=6.15 Hz, 1H) 12.35 (br. s., 1H). ¹³C NMR (100 MHz, DMSO-d₆) δ ppm 29.64, 41.55, 60.38, 101.29, 116.38, 119.07, 125.12, 130.58, 138.37, 140.05. m/z (ESI-MS) [M]⁺ 205.2.

### 2-Neopentyl-4-chloro-7-azaindole

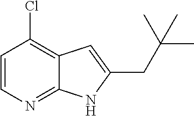

In sealed tube, 2-neopentyl-7-oxide-7-azaindole (0.9 g, 4.4 mmol) was dissolved in phosphoryl chloride (1.2 ml, 13.2 mmol) and the reaction heated to 90° C. and stirred to 18 h. The reaction mixture then warmed to room temperature and poured in to water and neutralized with saturated sodium carbonate solution and extract with EtOAc. The collected organic layer dried over magnesium sulfate, concentrated and triturated EtOAc in petroleum ether to afford the titled compound as brown solid (270 mg, 27%). ¹H NMR (400 MHz, DMSO-d₆) δ ppm 0.94 (s, 9H) 2.62 (s, 2H) 6.18 (d, J=1.76 Hz, 1H) 7.12 (d, J=5.27 Hz, 1H) 8.07 (d, J=5.27 Hz, 1H) 11.83 (br. s., 1H). ¹³C NMR (100 MHz, DMSO-d₆) δ ppm 29.77, 31.97, 42.23, 97.23, 115.56, 119.81, 132.83, 140.56, 142.30, 149.33. m/z (ESI-MS) [M]⁺ 223.1.

### 5-(2-Neopentyl-1H-pyrrolo[2,3-b]pyridin-4-yl)-1H-indazol-3-amine

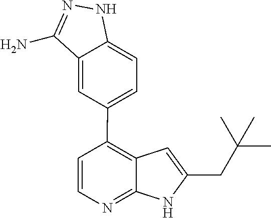 [SMILES: CC(C)(C)Cc1cc2c(-c3ccc4[nH]nc(N)c4c3)ccnc2[nH]1]

4-Chloro-2-neopentyl-7-azaindole (0.08 g, 0.36 mmol), 5-(4,4,5,5-tetramethyl-1,3,2-dioxaborolan-2-yl)-1H-indazol-3-amine (0.14 g, 0.54 mmol), 1M potassium phosphate solution (0.72 ml, 0.72 mmol), [1,1′-bis(di-tert-butylphosphino)ferrocene]dichloropalladium(II) catalyst (0.011 g, 0.018 mmol) in 2.16 ml ethanol were reacted as described in general procedure and chromatographic purification (90% EtOAc and 1% triethylamine in petroleum ether 60-80%), gave the titled compound as white solid (89.8 mg, 86%). ¹H NMR (400 MHz, DMSO-d₆) δ ppm 0.95 (s, 9H) 2.63 (s, 2H) 5.51 (s, 2H) 6.39 (s, 1H) 7.12 (d, J=4.83 Hz, 1H) 7.38 (d, J=8.79 Hz, 1H) 7.65 (dd, J=8.57, 1.54 Hz, 1H) 8.13 (s, 1H) 8.16 (d, J=5.27 Hz, 1H) 11.51 (s, 1H) 11.55 (s, 1H). ¹³C NMR (100 MHz, DMSO-d₆) δ ppm 29.88, 32.00, 42.47, 98.80, 110.27, 114.36, 115.07, 118.56, 120.59, 127.12, 128.71, 139.18, 140.38, 141.56, 142.05, 149.57, 150.26. m/z (ESI-HRMS) calculated for C₁₉H₂₂N₅₌320.1870 found=320.1867.

### Example 221

### 2-(Cyclohexylmethyl)-7-azaindole

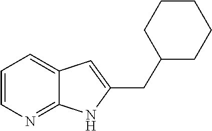 [SMILES: c1cnc2[nH]c(CC3CCCCC3)cc2c1]

To a stirred solution of 2(Boc-amino)-3-methyl-pyridine (1.77 g, 7.97 mmol) in anhydrous THF (40 ml) at −4° C., 2M n-butyllithium (10 ml, 20 mmol) was added slowly over 15 min. Then, 2-cyclohexyl-N-methoxy-N-methylacetamide (1.5 g, 8.7 mmol) was added slowly and the reaction mixture was stirred for 2 h. Thereafter, hydrochloric acid (5M, 50 ml) was added and the reaction was heated to 60° C. and stirred for 2 h. The organic layer was separated and the aqueous layer was neutralized and extracted with EtOAc. The collected organic layer was washed with brine, dried over magnesium sulfate, and evaporated. The crude product was then purified using column chromatography (15% EtOAc in petroleum ether 60-80%) to give the product as a white solid (1.1 g, 69%). 1H NMR (400 MHz, DMSO-d₆) δ ppm 0.88-1.02 (m, 2H) 1.09-1.25 (m, 3H) 1.55-1.68 (m, 6H) 2.59 (d, J=7.03 Hz, 2H) 6.11 (d, J=1.76 Hz, 1H) 6.96 (dd, J=7.91, 4.83 Hz, 1H) 7.77 (dd, J=7.91, 1.32 Hz, 1H) 8.07 (dd, J=4.83, 1.76 Hz, 1H) 11.41 (br. s., 1H). ¹³C NMR (100 MHz, DMSO-d₆) δ ppm 26.20, 26.54, 33.19, 36.19, 37.90, 98.12, 115.72, 121.16, 127.04, 140.41, 141.53, 149.22. m/z (ESI-MS) [M]⁺ 215.1.

### 2-(Cyclohexylmethyl)-7-oxide-7-azaindole

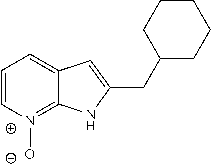 [SMILES: [O-][n+]1cccc2cc(CC3CCCCC3)[nH]c21]

To an ice-cooled solution of 2-(cyclohexylmethyl)-7-azaindole (0.83 g, 4.18 mmol) in EtOAc, meta-chloroperoxybenzoic acid (1.1 g, 6.7 mmol) was added slowly. Then, the reaction was warmed to room temperature and stirred to 1 h. After the reaction was completed, the solvent was evaporated, treated, with 1M sodium carbonate solution, and extracted with EtOAc. The residue was then concentrated under vacuum to give the product as a yellow solid. (0.81 g, 89%). ¹H NMR (400 MHz, DMSO-d₆) δ ppm 0.83-0.98 (m, 2H) 1.03-1.16 (m, 3H) 1.59-1.62 (m, 6H) 2.60 (d, J=6.59 Hz, 2H) 6.26 (s, 1H) 7.01 (dd, J=7.91, 6.15 Hz, 1H) 7.51 (d, J=7.91 Hz, 1H) 8.04 (d, J=6.15 Hz, 1H) 12.41 (br. s., 1H). ¹³C NMR (100 MHz, DMSO-d₆) δ ppm 26.17, 26.48, 33.02, 35.63, 37.97, 100.36, 116.51, 119.12, 125.28, 130.64, 138.74, 141.40. m/z (ESI-MS) [M]⁺ 231.2.

### 2-(Cyclohexylmethyl)-4-chloro-7-azaindole

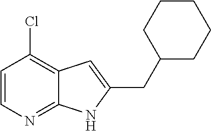 [SMILES: Clc1ccnc2[nH]c(CC3CCCCC3)cc12]

In a sealed tube, 2-(cyclohexylmethyl)-7-oxide-7-azaindole (0.8 g, 3.7 mmol) was dissolved in phosphoryl chloride (1.04 ml, 11.2 mmol). Next, the reaction mixture was heated to 90° C. and stirred for 18 h. Then, the reaction mixture was warmed to room temperature, poured into water, neutralized with saturated sodium carbonate solution, and extracted with EtOAc. The collected organic layer was dried over magnesium sulfate, concentrated and purified using column chromatography (30% EtOAc in petroleum ether 60-80%) to afford the titled compound as a white solid. (0.56 g, 64%), ¹H NMR (400 MHz, DMSO-d₆) δ ppm 0.85-1.03 (m, 2H) 1.05-1.28 (m, 3H) 1.53-1.78 (m, 6H) 2.62 (d, J=7.03 Hz, 2H) 6.19 (s, 1H) 7.11 (d, J=5.27 Hz, 1H) 8.05 (d, J=5.27 Hz, 1H) 11.83 (br. s., 1H). ¹³C NMR (100 MHz, DMSO-d₆) δ ppm 26.17, 26.50, 33.14, 35.63, 37.84, 96.30, 115.64, 119.95, 132.82, 141.92, 142.32, 149.75. m/z (ESI-MS) [M]⁺ 249.1

### 5-(2-(Cyclohexylmethyl)-1H-pyrrolo[2,3-b]pyridin-4-yl)-1H-indazol-3-amine

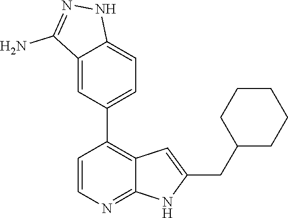 [SMILES: Nc1n[nH]c2ccc(-c3ccnc4[nH]c(CC5CCCCC5)cc34)cc12]

2-(Cyclohexylmethyl)-4-chloro-7-azaindole (0.08 g, 0.34 mmol), 5-(4,4,5,5-tetramethyl-1,3,2-dioxaborolan-2-yl)-1H-indazol-3-amine (0.13 g, 0.51 mmol), 1M potassium phosphate solution (0.6 ml, 0.6 mmol), [1,1′-bis(di-tert-butylphosphino)ferrocene]dichloropalladium(II) catalyst (0.004 g, 0.017 mmol) in 2 ml ethanol were reacted as described in General procedure A and chromatographic purification (70% EtOAc in petroleum ether 60-80%), gave the titled compound as yellow solid (71.8 mg, 61%). ¹H NMR (400 MHz, DMSO-d₆) δ ppm 0.88-1.04 (m, 2H) 1.08-1.27 (m, 3H) 1.54-1.75 (m, 6H) 2.63 (d, J=6.59 Hz, 2H) 5.52 (s, 2H) 6.41 (d, J=1.76 Hz, 1H) 7.11 (d, J=5.27 Hz, 1H) 7.37 (d, J=8.79 Hz, 1H) 7.64 (dd, J=8.79, 1.32 Hz, 1H) 8.08-8.17 (m, 2H) 11.52 (s, 1H) 11.54 (s, 1H). ¹³C NMR (100 MHz, DMSO-d₆) 26.22, 26.54, 33.24, 36.31, 38.03, 97.86, 110.34, 114.40, 115.14, 118.66, 120.67, 127.22, 128.73, 140.34, 140.52, 141.63, 142.06, 149.98, 150.37. m/z (ESI-HRMS) calculated for C₂₁H₂₄N₅=346.2026 found=346.2028.

### Example 222

### 2-Cyclohexylpropyl-7-azaindole

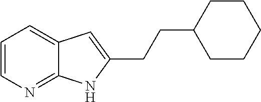 [SMILES: c1cnc2[nH]c(CCC3CCCCC3)cc2c1]

To a stirred solution of 2(Boc-amino)-3-methyl-pyridine (0.87 g, 4.1 mmol) in anhydrous THF (30 ml) at −4° C., 2M n-butyllithium (5.1 ml, 10.25 mmol) was added slowly over 15 min, then 3-cyclohexyl-N-methoxy-N-methylpropanamide (1 g, 5 mmol) was added slowly and the reaction mixture allowed to stirred for 2 h. Thereafter, hydrochloric acid (5M, 30 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated. The crude then purified using column chromatography (15% EtOAc in petroleum ether 60-80%) to give the product as white solid (0.38 g, 40%). ¹H NMR (400 MHz, DMSO-d₆) δ ppm 0.87-0.99 (m, 2H) 1.08-1.23 (m, 4H) 1.23-1.31 (m, 1H) 1.54-163 (m, 2H) 1.63-1.71 (m, 2H) 1.75 (d, J=12.97 Hz, 2H) 2.73 (t, J=7.86 Hz, 2H) 6.06-6.19 (m, 1H) 6.97 (dd, J=7.78, 473 Hz, 1H) 7.67-7.88 (m, 1H) 8.08 (dd, J=4.73, 1.53 Hz, 1H) 11.41 (br. s., 1H). ¹³C NMR (100 MHz, DMSO-d₆) δ ppm 25.62, 26.23, 26.63, 33.10, 36.45, 37.03, 96.98, 115.62, 121.06, 126.98, 141.47, 141.99, 149.18. m/z (ESI-MS) [M]⁺ 229.1.

### 2-Cyclohexylethyl-7-oxide-7-azaindole

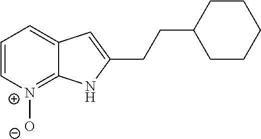 [SMILES: [O-][n+]1cccc2cc(CCC3CCCCC3)[nH]c21]

To an ice-cool solution of 2-cyclohexylethyl-7-azaindole (0.34 g, 1.5 mmol) in EtOAc, meta-chloroperoxybenzoic acid (0.41 g, 2.5 mmol) was added slowly then the reaction warmed to room temperature and stirred to 1 h. After the reaction was completed, the solvent was evaporated, treated with 1M sodium carbonate solution and extracted with EtOAc. The residue then concentrated under vacuum to give the product as a yellow solid (0.4 g, 99%). ¹H NMR (400 MHz, DMSO-d₆) δ ppm 0.86-0.99 (m, 2H) 1.10-1.28 (m, 4H) 1.55-1.63 (m, 4H) 1.67 (d, J=12.05 Hz, 2H) 1.73 (d, J=12.82 Hz, 2H) 2.74 (t, J=7.78 Hz, 1H) 6.28 (s, 1H) 7.00 (dd, J=7.86, 6.18 Hz, 1H) 7.49 (d, J=7.78 Hz, 1H) 8.01 (d, J=6.26 Hz, 1H) 12.27 (br. s., 1H). ¹³C NMR (100 MHz, DMSO-d₆) δ 25.25, 26.20, 26.62, 33.05, 36.43, 36.69, 99.28, 116.41, 118.82, 125.11, 130.56, 142.91, 157.14. m/z (ESI-MS) [M]⁺ 245.1.

### 4-Chloro-2-(2-cyclohexylethyl)-7-azaindole

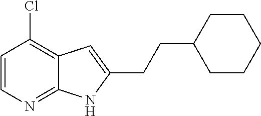 [SMILES: Clc1ccnc2[nH]c(CCC3CCCCC3)cc12]

In sealed tube, 2-cyclohexylethyl-7-oxide-7-azaindole (0.4 g, 1.6 mmol) was dissolved in phosphoryl chloride (0.45 ml, 4.9 mmol) and the reaction heated to 90° C. and stirred to 18 h. The reaction mixture then warmed to room temperature and poured in to water and neutralized with saturated sodium carbonate solution and extract with EtOAc. The collected organic layer dried over magnesium sulfate, concentrated and triturated EtOAc in petroleum ether to afford the titled compound as brown solid (307 mg, 73%). ¹H NMR (400 MHz, DMSO-d₆) δ ppm 0.88-0.98 (m, 2H) 1.10-1.31 (m, 4H) 1.56-1.71 (m, 5H) 1.75 (d, J=12.66 Hz, 2H) 2.72-2.78 (m, 2H) 6.19-6.23 (m, 1H) 7.11 (d, J=5.19 Hz, 1H) 8.06 (d, J=5.19 Hz, 1H) 11.85 (br. s., 1H). ¹³C NMR (100 MHz, DMSO-d₆) δ ppm 25.59, 26.21, 26.62, 33.06, 36.31, 37.06, 95.19, 115.55, 119.86, 132.78, 142.25, 143.53, 149.71. m/z (ESI-MS) [M]⁺ 263.1.

### 5-(2-(2-Cyclohexylethyl)-1H-pyrrolo[2,3-b]pyridin-4-yl)-1H-indazol-3-amine

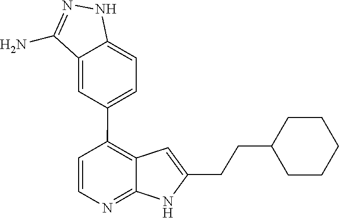 [SMILES: Nc1n[nH]c2ccc(-c3ccnc4[nH]c(CCC5CCCCC5)cc34)cc12]

4-Chloro-2-cyclohexylethyl-7-azaindole (0.075 g, 0.25 mmol), 5-(4,4,5,5-tetramethyl-1,3,2-dioxaborolan-2-yl)-1H-indazol-3-amine (0.1 g, 0.41 mmol), 1M potassium phosphate solution (0.5 ml, 0.5 mmol), [1,1′-bis(di-tert-butylphosphino)ferrocene]dichloropalladium(II) catalyst (0.008 g, 0.012 mmol) in 1.5 ml ethanol were reacted as described in general procedure and chromatographic purification (60% EtOAc and 1% triethylamine in petroleum ether 60-80%), gave the titled compound as white solid (60 mg, 66%). ¹H NMR (400 MHz, DMSO-d₆) δ ppm 0.87-0.98 (m, 2H) 1.10-1.32 (m, 5H) 1.56-1.70 (m, 4H) 1.76 (d, J=12.51 Hz, 2H) 2.72-2.81 (m, 2H) 5.50 (s, 2H) 6.39-6.45 (m, 1H) 7.11 (d, J=5.04 Hz, 1H) 7.38 (d, J=8.70 Hz, 1H) 7.65 (dd, J=8.70, 1.53 Hz, 1H) 8.10-8.17 (m, 2H) 11.53 (m, 2H). ¹³C NMR (100 MHz, DMSO-d₆) δ ppm 25.80, 26.22, 26.63, 37.19, 33.10, 36.59, 96.96, 110.24, 114.33, 115.05, 118.56, 120.57, 127.10, 128.64, 140.3, 1141.55, 141.99, 142.09, 149.95, 150.26. m/z (ESI-HRMS) calculated for C₂₁H₂₅N₆=361.2135 found=361.2132.

### Example 223

### 4-Chloro-3-(3-phenylprop-1-yn-1-yl}pyridin-2-amine

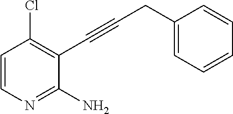 [SMILES: Nc1nccc(Cl)c1C#CCc1ccccc1]

A solution of 4-chloro-3-iodo-pyridine-2-amine (0.15 g, 0.6 mmol), copper (1) iodide (0.005 g, 0.03 mmol) and bis(triphenylphosphine) palladium(II) chloride (0.02 g, 0.03 mmol) in 3 ml of N,N-dimethyformamide-triethylamine (1:4) was degassed in sealed tube followed by addition of prop-2-yn-1-ylbenzene (0.1 ml, 0.88 mmol). The reaction mixture was stirred at 80° C. for 4 h. The reaction mixture was diluted with EtOAc and washed with 1 M sodium carbonate. The extracted organic layer was washed with brine and dried over magnesium sulfate and concentrated under reduced pressure and the residue purified by column chromatography (5-15% EtOAc in petroleum ether 60-80%) to give the product as yellow solid (0.095 g, 64%). ¹H NMR (400 MHz, DMSO-d₆) δ ppm 4.00 (s, 2H) 6.52 (br. s., 2H) 6.69 (d, J=5.71 Hz, 1H) 7.31-7.35 (m, 2H) 7.35-7.39 (m, 1H) 7.42-7748 (m, 2H) 7.85 (d, J=5.71 Hz, 1H). m/z (ESI-MS) [M]⁺ 243.1.

### 2-Benzyl-4-chloro-7-azaindole

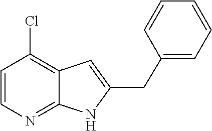 [SMILES: Clc1ccnc2[nH]c(Cc3ccccc3)cc12]

To a solution of 4-chloro-3-(3-phenylprop-1-yn-1-yl)pyridin-2-amine (0.087 g, 0.36 mmol) in 1,4 dioxane (2 ml), potassium tert-butoxide (0.12 g, 1 mmol) and 18-crown-6 (0.009 g, 10%, 0.036 mmol) were added and the mixture was stirred at 110° C. for 18 h. After the reaction was cooled to room temperature, EtOAc and 1M sodium carbonate solution were added. Extracted organic layer was dried over magnesium sulfate and concentrated under reduced pressure to give the product as yellow solid (76.7 mg, 88%). ¹H NMR (400 MHz, DMSO-d₆) δ ppm 4.10 (s, 2H) 6.18 (s, 1H) 7.12 (d, J=5.27 Hz, 1H) 7.20-7.27 (m, 1H) 7.31-7.37 (m, 4H) 8.07 (d, J=5.27 Hz, 1H) 12.10 (br. s., 1H). ¹³C NMR (100 MHz, DMSO-d₆) δ ppm 34.45, 96.39, 115.78, 119.87, 126.99, 129.03, 129.31, 133.24, 139.22, 142.08, 142.79, 149.93. m/z (ESI-MS) [M]⁺ 243.0.

### 5-(2-Benzyl-1H-pyrrolo[2,3-b]pyridin-4-yl)-1H-indazol-3-amine

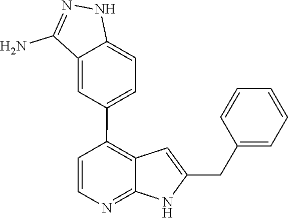 [SMILES: Nc1n[nH]c2ccc(-c3ccnc4[nH]c(Cc5ccccc5)cc34)cc12]

2-Benzyl-4-chloro-7-azaindole (0.02 g, 0.08 mmol), 5-(4,4,5,5-tetramethyl-1,3,2-dioxaborolan-2-yl)-1H-indazol-3-amine (0.031 g, 0.12 mmol), 1M potassium phosphate solution (0.16 ml, 0.16 mmol), [1,1′-bis(di-tert-butylphosphino)ferrocene]dichloropalladium(II) catalyst (0.002 g, 0.004 mmol) in 0.5 ml ethanol were reacted as described in General procedure A and HPLC purification gave the titled compound as yellow solid (22 mg, 48%). ¹H NMR (400 MHz, DMSO-d₆) δ ppm 4.20 (s, 2H) 6.62 (s, 1H) 7.25-7.30 (m, 1H) 7.31 (d, J=5.19 Hz, 1H) 7.37 (t, J=7.63 Hz, 2H) 7.40 (d, J=7.02, 2H) 7.52 (d, J=8.85 Hz, 1H) 7.81 (d, J=8.70 Hz, 1H) 8.29-8.33 (m, 2H) 12.10 (br. s, 1H). ¹³C NMR (100 MHz, DMSO-d₆) δ ppm 34.44, 98.56, 110.98, 114.56, 115.44, 119.84, 121.40, 126.49, 126.83, 128.89, 128.91, 129.11, 136.09, 139.40, 141.34, 141.89, 144.31, 151.35, 152.76. m/z (ESI-HRMS) calculated for C₂₁H₁₈N₅₌340.1550 found=340.1557.

### Example 224

### 4-(3-Amino-1H-indazol-5-yl)-1H-pyrrolo[2,3-d]pyridin-2-yl)methanol

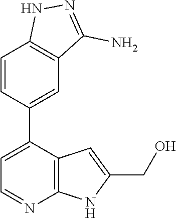 [SMILES: Nc1n[nH]c2ccc(-c3ccnc4[nH]c(CO)cc34)cc12]

4-(Chloro-1H-pyrrolo[2,3-b]pyridin-2-yl)methanol (0.11 g, 0.60 mmol), 5-(4,4,5,5-tetramethyl-1,3,2-dioxaborolan-2-yl)-1H-indazol-3-amine (0.258 g, 1.00 mmol) and [1,1′-bis(di-tert-butylphosphino)ferrocene]dichloropalladium(II) catalyst (0.040 g, 0.06 mmol) in EtOH (3.0 mL, degassed under nitrogen) was added K₃PO₄ (1M, 1.2 mL) and the reaction mixture was heated to 100° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was dissolved in DMF (1.5 mL) and passed through a pad of cotton wool prior to being purified by HPLC. Purification of 0.02 g of crude material by HPLC (t_{R}=6 min) afforded the title compound as the double TFA salt (0.0052 g, 16.9%). ¹H NMR (DMSO-d₆): δ 4.67 (s, 2H), 6.68 (s, 1H), 7.29 (d, J=5.2 Hz, 1H), 7.47 (d, J=8.8 Hz, 1H), 7.77 (dd, J=1.6, 8.8 Hz, 1H), 8.27 (m, 2H), 11.97 (br s, 1H). HRMS: For C₁₅H₁₄ON₅ requires 280.1190 found 280.1193. Analytical LCMS @254 nm t_{R}=8.88 min; 87.9%; MS (ESI) m/z (M+H)+.

### Example 225

### 2-(4-(3-Amino-1H-indazol-5-yl)-1H-pyrrolo[2,3-b]pyridin-2-yl)propan-2-ol

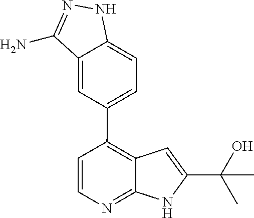 [SMILES: CC(C)(O)c1cc2c(-c3ccc4[nH]nc(N)c4c3)ccnc2[nH]1]

2-(4-Chloro-1H-pyrrolo[2,3-b]pyridin-2-yl)propan-2-ol (0.105 g, 0.5 mmol), 5-(4,4,5,5-tetramethyl-1,3,2-dioxaborolan-2-yl)-1H-indazol-3-amine (0.194 g, 0.75 mmol) and [1,1′-bis(di-tert-butylphosphino)ferrocene]dichloropalladium(II) catalyst (0.016 g, 0.025 mmol) in EtOH (3.0 mL, degassed under nitrogen) was added K₃PO₄ (1M, 1.2 mL) and the reaction mixture was heated to 100° C. for 22 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was dissolved in DMF (1.5 mL) and passed through a pad of cotton wool prior to being purified by HPLC to afford the title compound as the double TFA salt (0.0551 g, 20%).

### Example 226

### 3-(4-(3-Amino-1H-indazol-5-yl)-1H-pyrrolo[2,3-d]pyridin-2-yl)pentan-3-ol

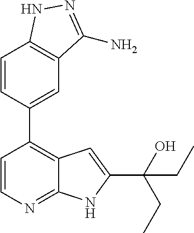 [SMILES: CCC(O)(CC)c1cc2c(-c3ccc4[nH]nc(N)c4c3)ccnc2[nH]1]

3-(4-Chloro-1H-pyrrolo[2,3-b]pyridin-2-yl)pentan-3-ol (0.105 g, 0.44 mmol), 5-(4,4,5,5-tetramethyl-1,3,2-dioxaborolan-2-yl)-1H-indazol-3-amine (0.21 g, 0.80 mmol) and [1,1′-bis(di-tert-butylphosphino)ferrocene]dichloropalladium(II) catalyst (0.029 g, 0.044 mmol) in EtOH (3.0 mL, degassed under nitrogen) was added K₃PO₄ (1M, 1.2 mL) and the reaction mixture was heated to 100° C. for 22 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was dissolved in DMF (1.5 mL) and passed through a pad of cotton wool prior to being purified by HPLC. Purification by HPLC (t_{R}=11 min) afforded the title compound as the double TFA salt (0.0451 g, 18.2%). ¹H NMR (DMSO-d₆): δ 0.73 (m, 6H), 1.83 (m, 4H), 6.61 (d, J=2.0 Hz, 1H), 7.29 (d, J=5.6 Hz, 1H), 7.51 (d, J=8.8 Hz, 1H), 7.80 (dd, J=1.6, 8.8 Hz, 1H), 8.27 (m, 2H), 11.90 (br s, 1H). HRMS: For C₁₉H₂₂ON₅ requires 336.1819 found 336.1813.

### Example 227

### 2-tert-Butoxymethy-4-chloro-7-azaindole

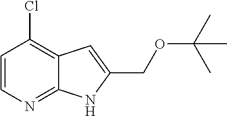 [SMILES: CC(C)(C)OCc1cc2c(Cl)ccnc2[nH]1]

To a solution of 3-(3-(tert-butoxy)prop-1-yn-1-yl)-4-chloropyridin-2-amine (0.6 g, 0.25 mmol) in 1,4 dioxane (2 ml), potassium tert-butoxide (0.07 g, 0.63 mmol) and 18-crown-6 (0.006 g, 10%, 0.0025 mmol) were added and the mixture was stirred at 110° C. for 18 h. After the reaction was cooled to room temperature, EtOAc and 1 M sodium carbonate solution were added. Extracted organic layer was dried over magnesium sulfate and concentrated under reduced pressure to give the product as yellow solid (31.7 mg, 96%). ¹H NMR (400 MHz, DMSO-d₆) δ ppm 1.24 (s, 9H) 4.54 (s, 2H) 6.38 (s, 1H) 7.15 (d, J=5.19 Hz, 1H) 8.12 (d, J=5.19 Hz, 1H) 11.98 (br. s., 1H). ¹³C NMR (100 MHz, DMSO-d₆) δ ppm 27.83, 40.27, 57.64, 96.45, 115.72, 115.68, 119.34, 140.50, 143.20, 143.19. m/z (ESI-MS)

### 5-(2-(tert-Butoxymethyl)-1H-pyrrolo[2,3-b]pyridin-4-yl)-1H-indazol-3-amine

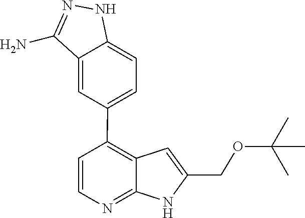 [SMILES: CC(C)(C)OCc1cc2c(-c3ccc4[nH]nc(N)c4c3)ccnc2[nH]1]

4-Chloro-2-tert-butoxymethyl-7-azaindole (0.03 g, 0.12 mmol), 5-(4,4,5,5-tetramethyl-1,3,2-dioxaborolan-2-yl)-1H-indazol-3-amine (0.05 g, 0.19 mmol), 1M potassium phosphate solution (0.25 ml, 0.25 mmol), [1,1′-bis(di-tert-butylphosphino)ferrocene]dichloropalladium(II) catalyst (0.004 g, 0.006 mmol) in 0.7 ml ethanol were reacted as described in General procedure A and chromatographic purification (90% EtOAc and 1% triethylamine in petroleum ether 60-80%), gave the titled compound as white solid (22 mg, 52%). ¹H NMR (400 MHz, DMSO-d₆) δ ppm 1.26 (s, 9H) 4.55 (s, 2H) 5.51 (s, 2H) 6.59 (s, 1H) 7.15 (d, J=5.04 Hz, 1H) 7.38 (d, J=8.70 Hz, 1H) 7.65 (dd, J=8.62, 1.6 Hz, 1H) 8.15 (s, 1H) 8.21 (d, J=5.04 Hz, 1H) 11.53 (s, 1H) 11.66 (s, 1H). ¹³C NMR (100 MHz, DMSO-d₆) δ ppm 27.88, 40.27, 57.85, 98.37, 110.29, 114.46, 115.05, 118.03, 120.69, 127.12, 128.45, 138.98, 141.34, 141.59, 143.00, 150.01, 150.29. m/z (ESI-HRMS) calculated for C₁₉H₂₂ON₅=336.1819 found=336.1815.

### Example 228

### 2-((Tetrahydro-2H-pyran-4-yl)-7-azaindole

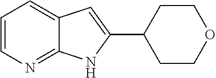 [SMILES: c1cnc2[nH]c(C3CCOCC3)cc2c1]

To stirred solution of 2(Boc-amino)-3-methyl-pyridine (0.55 g, 2.6 mmol) in anhydrous THF (20 ml) at −4° C., 2M n-butyllithium (3.2 ml, 6.5 mmol) was added slowly over 15 min, then N-methoxy-N-methyl-2-(tetrahydro-2H-pyran-4-yl)carboxamamide (0.5 g, 2.9 mmol) was added slowly and the reaction mixture allowed to stirred for 2 h. Therafter, hydrochloric acid (5M, 19 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated to give the product as white solid (0.51 g, 84%). ¹H NMR (400 MHz, DMSO-d₆) δ ppm 1.64-1.79 (m, 2H) 1.87-1.97 (m, 2H) 2.97 (tt, J=11.75, 3.63 Hz, 1H) 3.45 (td, J=11.64, 2.20 Hz, 2H) 3.94 (dd, J=11.42, 2.64 Hz, 2H) 6.11-6.19 (m, 1H) 6.98 (dd, J=7.91, 4.83 Hz, 1H) 7.81 (dd, J=7.91, 1.32 Hz, 1H) 8.11 (dd, J=4.83, 1.76 Hz, 1H) 11.53 (br. s., 1H). ¹³C NMR (100 MHz, DMSO-d₆) δ ppm 32.28, 34.58, 67.34, 95.46, 115.72, 120.69, 127.43, 141.86, 145.23, 149.20. m/z (ESI-MS) [M]⁺ 203.1.

### 2-(Tetrahydro-2H-pyran-4-yl)-7-oxide-7-azaindole

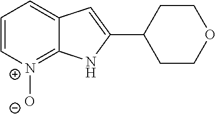 [SMILES: [O-][n+]1cccc2cc(C3CCOCC3)[nH]c21]

To an ice-cooled solution of 2-((tetrahydro-2H-pyran-4-yl)-7-azaindole (0.55 g, 2.4 mmol) in EtOAc, meta-chloroperoxybenzoic acid (0.68 g, 3.9 mmol) was added slowly then the reaction warmed to room temperature and stirred to 1 h. After the reaction was completed, the solvent was evaporated, treated with 1 M sodium carbonate solution and extracted with EtOAc. The residue then concentrated under vacuum to give the product as a yellow solid (0.4 g, 76%). ¹H NMR (400 MHz, DMSO-d₆) δ ppm 1.64-1.77 (m, 2H) 1.89-1.96 (m, 2H) 2.99 (tt, J=11.75, 3.63 Hz, 1H) 3.42 (td, J=11.64, 1.76 Hz, 2H) 3.93 (dd, J=11.42, 3.08 Hz, 2H) 6.31 (s, 1H) 7.02 (dd, J=7.91, 6.15 Hz, 1H) 7.53 (d, J=7.91 Hz, 1H) 8.05 (d, J=6.15 Hz, 1H) 12.35 (br. s., 1H). ¹³C NMR (100 MHz, DMSO-d₆) δ ppm 32.19, 34.40, 67.32, 97.81, 116.51, 119.27, 124.81, 130.91, 138.86, 146.15. m/z (ESI-MS) [M]⁺ 219.1.

### 2-(Tetrahydro-2H-pyran-4-yl)-4-chloro-7-azaindole

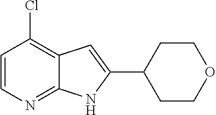 [SMILES: Clc1ccnc2[nH]c(C3CCOCC3)cc12]

In sealed tube, 2-(tetrahydro-2H-pyran-4-yl)-7-oxide-7-azaindole (0.35 g, 1.6 mmol) was dissolved in phosphoryl chloride (0.4 ml, 4.8 mmol) and the reaction heated to 90° C. and stirred to 18 h. The reaction mixture then warmed to room temperature and poured into water and neutralized with saturated sodium carbonate solution and extract with EtOAc. The collected organic layer dried over magnesium sulfate and concentrated. The crude then purified using column chromatography (40-60% EtOAc in petroleum ether 60-80%) to give the product as white solid (68.6 mg, 18%). ¹H NMR (400 MHz, DMSO-d₆) δ ppm 11.69-1.80 (m, 2H) 1.94 (dd, J=12.82, 1.83 Hz, 2H) 3.01 (tt, J=11.67, 3.74 Hz, 1H) 3.46 (td, J=11.67, 1.83 Hz, 2H) 3.95 (dd, J=10.83, 3.20 Hz, 2H) 6.24 (d, J=1.53 Hz, 1H) 7.13 (d, J=5.19 Hz, 1H) 8.10 (d, J=5.34 Hz, 1H) 11.93 (br. s., 1H). ¹³C NMR (100 MHz, DMSO-d₆) δ ppm 32.09, 34.58, 67.28, 93.73, 115.63, 119.52, 133.26, 142.64, 146.57, 149.75. m/z (ESI-MS) [M]⁺ 237.1.

### 5-(2-(tetrahydro-2H-pyran-4-yl)-1H-pyrrolo[2,3-b]pyridin-4-yl)-1H-indazol-3-amine

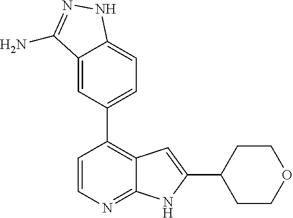 [SMILES: Nc1n[nH]c2ccc(-c3ccnc4[nH]c(C5CCOCC5)cc34)cc12]

4-Chloro-2-(tetrahydro-2H-pyran-4yl)-7-azaindole (0.048 g, 0.2 mmol), 5-(4,4,5,5-tetramethyl-1,3,2-dioxaborolan-2-yl)-1H-indazol-3-amine (0.08 g, 0.3 mmol), 1M potassium phosphate solution (0.4 ml, 0.4 mmol), [1,1′-bis(di-tert-butylphosphino)ferrocene]dichloropalladium(II) catalyst (0.006 g, 0.012 mmol) in 1.2 ml ethanol were reacted as described in General procedure A and chromatographic purification (18% methanol in EtOAc and 1% triethylamine), gave the titled compound as brown solid (56.5 mg, 84%). ¹H NMR (400 MHz, DMSO-d₆) b ppm 1.72-1.85 (m, 2H) 1.91-2.01 (m, 2H) 3.02 (tt, J=11.65, 3.68 Hz, 1H) 3.47 (td, J=11.63, 1.60 Hz, 2H) 3.96 (dd, J=11.22, 2.98 Hz, 2H) 5.51 (s, 2H) 6.44 (d, J=1.53 Hz, 1H) 7.13 (d, J=5.04 Hz, 1H) 7.38 (d, J=8.70 Hz, 1H) 7.65 (dd, J=8.54, 1.53 Hz, 1H) 8.14 (s, 1H) 8.17 (d, J=5.04 Hz, 1H) 11.53 (s, 1H) 11.62 (s, 1H). ¹³C NMR (100 MHz, DMSO-d₆) δ ppm 32.30, 34.73, 67.39, 95.17, 110.29, 114.39, 115.05, 118.18, 120.59, 127.12, 128.55, 140.77, 141.56, 142.36, 145.25, 149.98, 150.27. m/z (ESI-HRMS) calculated for C₁₉H₂₀ON₅=334.1662 found=334.1659.

### Example 229

### 2-((Tetrahydro-2H-pyran-2-yl)-7-azaindole

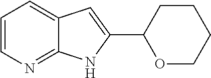 [SMILES: c1cnc2[nH]c(C3CCCCO3)cc2c1]

To stirred solution of 2(Boc-amino)-3-methyl-pyridine (0.55 g, 2.6 mmol) in anhydrous THF (20 ml) at −4° C., 2M n-butyllithium (3.2 ml, 6.5 mmol) was added slowly over 15 min, then N-methoxy-N-methyl-2-(tetrahydro-2H-pyran-4-yl)carboxamamide (0.5 g, 2.9 mmol) was added slowly and the reaction mixture allowed to stirred for 2 h. Thereafter, hydrochloric acid (5M, 19 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated to give the product as white solid (0.54 g, 84%). ¹H NMR (400 MHz, DMSO-d₆) δ ppm 1.75 (m, 3H) 1.86-1.98 (m, 2H) 3.52-3.62 (m, 1H) 3.97-4.03 (m, 1H) 4.52 (dd, J=10.76, 2.21 Hz, 1H) 6.31 (s, 1H) 7.02 (dd, J=7.78, 4.73 Hz, 1H) 7.87 (dd, J=7.78, 1.68 Hz, 1H) 8.16 (dd, J=4.65, 1.60 Hz, 1H) 11.59 (br. s., 1H). ¹³C NMR (100 MHz, DMSO-d₆) δ ppm 23.36, 25.91, 31.40, 68.26, 73.80, 96.65, 115.83, 120.19, 128.10, 141.77, 142.53, 148.99. m/z (ESI-MS) [M]⁺ 203.1.

### 2-(Tetrahydro-2H-pyran-2-yl)-7-oxide-7-azaindole

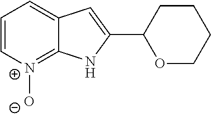 [SMILES: [O-][n+]1cccc2cc(C3CCCCO3)[nH]c21]

To an ice-cooled solution of 2-((tetrahydro-2H-pyran-2-yl)-7-azaindole (0.5 g, 2.4 mmol) in EtOAc, meta-chloroperoxybenzoic acid (0.68 g, 4 mmol) was added slowly then the reaction warmed to room temperature and stirred to 1 h. After the reaction was completed, the solvent was evaporated, treated with 1 M sodium carbonate solution and extracted with EtOAc. The residue then concentrated under vacuum to give the product as a yellow solid (0.5 g, 95%). ¹H NMR (400 MHz, DMSO-d₆) δ ppm 1.57 (d, J=7.28 Hz, 3H) 1.84-1.92 (m, 1H) 1.92-2.02 (m, 1H) 3.65 (t, J=6.78 Hz, 1H) 3.93-4.04 (m, 2H) 4.51 (dd, J=10.79, 2.01 Hz, 1H) 6.44 (s, 1H) 7.06 (s, 1H) 7.56 (s, 1H) 8.08 (s, 1H) 12.44 (br. s., 1H). ¹³C NMR (100 MHz, DMSO-d₆) b ppm 22.89, 25.29, 31.11, 67.87, 72.97, 98.31, 116.13, 119.25, 123.90, 127.59, 130.86, 142.25. m/z (ESI-MS) [M]⁺ 219.1.

### 2-(Tetrahydro-2H-pyran-2-yl)-4-chloro-7-azaindole

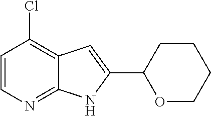 [SMILES: Clc1ccnc2[nH]c(C3CCCCO3)cc12]

In sealed tube, 2-(tetrahydro-2H-pyran-2-yl)-7-oxide-7-azaindole (0.35 g, 1.6 mmol) was dissolved in phosphoryl chloride (0.4 ml, 4.8 mmol) and the reaction heated to 90° C. and stirred to 18 h. The reaction mixture then warmed to room temperature and poured in to water and neutralized with saturated sodium carbonate solution and extract with EtOAc. The collected organic layer dried over magnesium sulfate and concentrated. The crude then purified using column chromatography (40-60% EtOAc in petroleum ether 60-80%) to give the product as white solid (41 mg, 7.5%). ¹H NMR (400 MHz, DMSO-d₆) δ ppm 1.57-1.80 (m, 3H) 1.84-189 (m, 1H) 1.90-3.05 (m, 2H) 3.46-3.61 (m, 1H) 3.95 (d, J=10.99 Hz, 1H) 4.50 (dd, J=10.98, 2.06 Hz, 1H) 6.24 (d, J=1.53 Hz, 1H) 7.13 (d, J=5.19 Hz, 1H) 8.09 (d, J=5.34 Hz, 1H) 11.93 (br. s., 1H). m/z (ESI-MS) [M]⁺ 237.1.

### 5-(2-(Tetrahydro-2H-pyran-2-yl)-1H-pyrrolo[2,3-b]pyridin-4-yl)-1H-indazol-3-amine

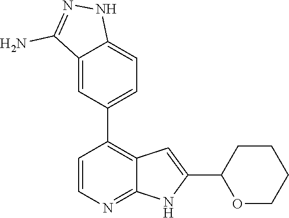 [SMILES: Nc1n[nH]c2ccc(-c3ccnc4[nH]c(C5CCCCO5)cc34)cc12]

4-Chloro-2-(tetrahydro-2H-pyran-2yl)-7-azaindole (0.033 g, 0.14 mmol), 5-(4,4,5,5-tetramethyl-1,3,2-dioxaborolan-2-yl)-1H-indazol-3-amine (0.054 g, 0.21 mmol), 1M potassium phosphate solution (0.28 ml, 0.28 mmol), [1,1′-bis(di-tert-butylphosphino)ferrocene]dichloropalladium(II) catalyst (0.004 g, 0.007 mmol) in 0.84 ml ethanol were reacted as described in General procedure A and chromatographic purification (18% methanol in EtOAc and 1% triethylamine), gave the titled compound as brown solid (28.3 mg, 60%). ¹H NMR (400 MHz, DMSO-d₆) b ppm 1.53-1.71 (m, 3H) 1.73-1.84 (m, 1H) 1.87-2.01 (m, 2H) 3.54-3.61 (m, 1H) 4.01 (d, J=10.99, 1H) 4.55 (dd, J=10.91, 2.06 Hz, 1H) 5.52 (s, 2H) 6.54 (d, J=1.53 Hz, 1H) 7.15 (d, J=5.04 Hz, 1H) 7.38 (d, J=8.54 Hz, 1H) 7.64 (dd, J=8.62, 1.60 Hz, 1H) 8.15 (s, 1H) 8.21 (d, J=4.88 Hz, 1H) 11.53 (s, 1H) 11.69 (s, 1H). ¹³C NMR (100 MHz, DMSO-d₆) δ ppm 23.41, 25.91, 31.47, 68.30, 73.96, 96.38, 110.30, 114.48, 115.05, 117.76, 120.67, 127.14, 128.41, 141.45, 141.60, 141.82, 143.01, 149.77, 150.29. m/z (ESI-HRMS) calculated for C₁₉H₂₀ON₅=334.1662 found=334.1660.

### Example 230

### 2-((Tetrahydro-2H-pyran-4-yl)methyl)-7-azaindole

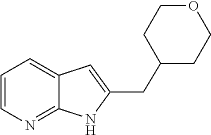 [SMILES: c1cnc2[nH]c(CC3CCOCC3)cc2c1]

To stirred solution of 2-(Boc-amino)-3-methyl-pyridine (1.5 g, 7.3 mmol) in anhydrous THF (40 ml) at −4° C., 2M n-butyllithium (9.1 ml, 18.25 mmol) was added slowly over 15 min, then N-methoxy-N-methyl-2-(tetrahydro-2H-pyran-4-yl)acetamide (1.5 g, 8 mmol) was added slowly and the reaction mixture allowed to stirred for 2 h. Thereafter, hydrochloric acid (5M, 50 ml) was added and the reaction was heated to 60° C. and stirred for 2 h. The organic layer was separated and aqueous layer was neutralized and extracted with EtOAc. The collected organic layer was washed with brine, dried over magnesium sulfate and concentrated. The crude then purified using column chromatography (40-60% EtOAc in petroleum ether 60-80%) to give the product as white solid (0.8 g, 50%). ¹H NMR (400 MHz, DMSO-d₆) δ ppm 1.19-1.29 (m, 2H) 1.53 (dd, J=12.89, 1.75 Hz, 2H) 1.94 (ttt, J=11.25, 11.25, 7.44, 7.44, 3.74, 3.74 Hz, 1H) 2.66 (d, J=7.17 Hz, 2H) 3.26 (td, J=7.67, 1.98 Hz, 2H) 3.83 (dd, J=11.44, 2.44 Hz, 2H) 6.16 (d, J=1.83 Hz, 1H) 6.97 (dd, J=7.71, 4.65 Hz, 1H) 7.79 (dd, J=7.78, 1.07 Hz, 1H) 8.09 (dd, J=4.65, 1.60 Hz, 1H) 11.43 (br. s., 1H). ¹³C NMR (100 MHz, DMSO-d₆) δ ppm 33.05, 35.16, 35.56, 67.39, 98.23, 115.68, 121.02, 127.05, 139.51, 141.58, 149.14. m/z (ESI-MS) [M]⁺ 217.1.

### 2-((Tetrahydro-2H-pyran-4-yl)methyl)-7-oxide-7-azaindole

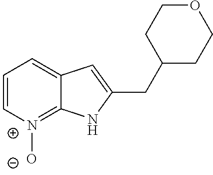 [SMILES: [O-][n+]1cccc2cc(CC3CCOCC3)[nH]c21]

To an ice-cooled solution of 2-((tetrahydro-2H-pyran-4-yl)methyl)-7-azaindole (0.35 g, 1.6 mmol) in EtOAc, meta-chloroperoxybenzoic acid (0.45 g, 2.6 mmol) was added slowly then the reaction warmed to room temperature and stirred to 1 h. After the reaction was completed, the solvent was evaporated, treated with 1M sodium carbonate solution and extracted with EtOAc. The residue then concentrated under vacuum to give the product as a yellow solid (0.35 g, 94%). ¹H NMR (400 MHz, DMSO-d₆) δ ppm 1.13-1.27 (m, 2H) 1.48 (dd, J=12.80, 1.76 Hz, 2H) 1.84-1.97 (m, 1H) 2.66 (d, J=7.28 Hz, 2H) 3.15-3.25 (m, 2H) 3.80 (dd, J=11.29, 2.51 Hz, 2H) 6.30 (s, 1H) 7.02 (dd, J=8.13, 6.37 Hz, 1H) 7.54 (d, J=7.91 Hz, 1H) 8.05 (d, J=6.15 Hz, 1H) 12.35 (br. s., 1H). ¹³C NMR (100 MHz, DMSO-d₆) δ ppm 32.38, 34.51, 34.71, 66.84, 100.00, 115.96, 118.60, 124.63, 130.16, 138.21, 139.99. m/z (ESI-MS) [M]⁺ 233.1.

### 2-((Tetrahydro-2H-pyran-4-yl)methyl)-4-chloro-7-azaindole

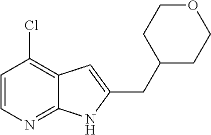 [SMILES: Clc1ccnc2[nH]c(CC3CCOCC3)cc12]

In sealed tube, 2-((tetrahydro-2H-pyran-4-yl)methyl)-7-oxide-7-azaindole (0.35 g, 1.5 mmol) was dissolved in phosphoryl chloride (0.4 ml, 4.5 mmol) and the reaction heated to 90° C. and stirred to 18 h. The reaction mixture then warmed to room temperature and poured in to water and neutralized with saturated sodium carbonate solution and extract with EtOAc. The collected organic layer dried over magnesium sulfate and concentrated. The crude then purified using column chromatography (40-60% EtOAc in petroleum ether 60-80%) to give the product as white solid (82.7 mg, 22%). ¹H NMR (400 MHz, DMSO-d₆) δ ppm 1.18-1.31 (m, 2H) 1.46-1.58 (m, 2H) 1.95 (dtt, J=11.25, 7.51, 7.51, 3.72, 3.72 Hz, 1H) 2.68 (d, J=7.32 Hz, 2H) 3.21-3.31 (m, 2H) 3.83 (dd, J=11.37, 2.82 Hz, 2H) 6.24 (d, J=1.83 Hz, 1H) 7.12 (d, J=5.19 Hz, 1H) 8.07 (d, J=5.19 Hz, 1H) 11.86 (br. s., 1H). ¹³C NMR (100 MHz, DMSO-d₆) δ ppm 32.98, 35.08, 35.43, 67.35, 96.41, 115.60, 119.84, 132.84, 141.02, 142.36, 149.67. m/z (ESI-MS) [M]⁺ 251.1.

### 5-(2-((tetrahydro-2H-pyran-4-yl)methyl)-1H-pyrrolo[2,3-b]pyridin-4-yl)-1H-indazol-3-amine

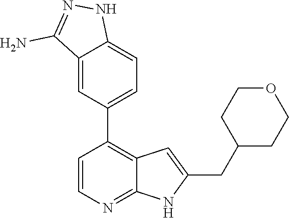 [SMILES: Nc1n[nH]c2ccc(-c3ccnc4[nH]c(CC5CCOCC5)cc34)cc12]

4-Chloro-2((tetrahydro-2H-pyran-4yl)methyl)-7-azaindole (0.065 g, 0.25 mmol), 5-(4,4,5,5-tetramethyl-1,3,2-dioxaborolan-2-yl)-1H-indazol-3-amine (0.1 g, 0.38 mmol), 1M potassium phosphate solution (0.5 ml, 0.5 mmol), [1,1′-bis(di-tert-butylphosphino)ferrocene]dichloropalladium(II) catalyst (0.008 g, 0.012 mmol) in 1.5 ml ethanol were reacted as described in general procedure and chromatographic purification (18% methanol in EtOAc and 1% triethylamine), gave the titled compound as brown solid (71.2 mg, 81%). ¹H NMR (400 MHz, DMSO-d₆) δ ppm 1.20-1.30 (m, 2H) 1.50-1.58 (m, 2H) 1.91-2.01 (m, 1H) 2.70 (d, J=7.17 Hz, 2H) 3.23-3.29 (m, 2H) 3.82 (dd, J=11.37, 2.67 Hz, 2H) 5.50 (s, 2H) 6.40-6.48 (m, 1H) 7.12 (d, J=5.04 Hz, 1H) 7.38 (d, J=8.70 Hz, 1H) 7.65 (dd, J=8.70, 1.53 Hz, 1H) 8.14 (s, 1H) 8.16 (d, J=4.88 Hz, 1H) 11.54 (br. s., 2H). ¹³C NMR (100 MHz, DMSO-d₆) δ ppm 33.07, 35.27, 35.68, 67.39, 97.97, 110.27, 114.35, 115.07, 118.55, 120.59, 127.12, 128.61, 139.60, 140.38, 141.57, 142.10, 149.91, 150.28. m/z (ESI-HRMS) calculated for C₂₀H₂₂ON₅=348.1819 found=348.1814.

### Example 231

### Methyl 4-(3-amino-1H-indazol-5-yl)-1H-pyrrolo[2,3-b]pyridine-2-carboxylate

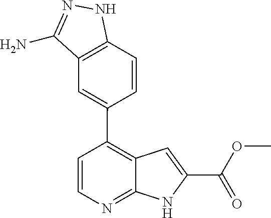 [SMILES: COC(=O)c1cc2c(-c3ccc4[nH]nc(N)c4c3)ccnc2[nH]1]

A 2-5 mL MW vial was charged with methyl 4-chloro-1H-pyrrolo[2,3-b]pyridine-2-carboxylate (0.210 g, 1 mmol), 5-(4,4,5,5-tetramethyl-1,3,2-dioxaborolan-2-yl)-1H-indazol-3-amine (0.337 g, 1.3 mmol), [1,1′-bis(di-tert-butylphosphino)ferrocene]dichloropalladium(II) catalyst (0.054 g, 0.208 mmol, 8% mol) and K₂CO₃ (0.248 g, 1.8 mmol, anhydrous). The vial was sealed with an aluminium crimp cap fitted with a disposable PTFE/silicon septum, and purged with nitrogen. The vial was then charged with MeOH (5 mL, anhydrous O₂-free) and the mixture was stirred at 45° C. and purged with N₂ for 15 min. The resulting mixture was then stirred to 70° C. for 13 hours. The reaction was then diluted with EtOAc (200 mL) and washed with H2O (30 mL×3). The organic phase was then dried (MgSO₄), filtered, absorbed onto silica and purified by flash chromatography (Biotage SP4, 50 g SiO₄, 0-3% MeOH in EtOAc) to afford the title compound as a green solid (0.045 g, 0.146 mmol, 15%). ¹H NMR (400 MHz, DMSO-d₆) δ 3.89 (s, 3H), 5.59 (s, 2H), 7.28 (d, J=4.9 Hz, 1H), 7.35-7.45 (m, 2H), 7.67 (d, J=8.7 Hz, 1H), 8.22 (s, 1H), 8.45 (d, J=4.9 Hz, 1H), 11.60 (s, 1H), 12.62 (s, 1H). HRMS: Calculated for C₁₆H₁₄O₂N₅ (M+H⁺)=308.1142; Found: 308.1142

### Example 232

### Ethyl 4-(3-amino-1H-indazol-5-yl)-1H-pyrrolo[2,3-b]pyridine-2-carboxylate

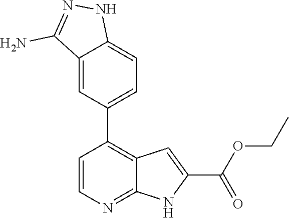 [SMILES: CCOC(=O)c1cc2c(-c3ccc4[nH]nc(N)c4c3)ccnc2[nH]1]

Ethyl 4-chloro-1H-pyrrolo[2,3-b]pyridin-2-carboxylate (0.11 g, 0.5 mmol), 5-(4,4,5,5-tetramethyl-1,3,2-dioxaborolan-2-yl)-1H-indazol-3-amine (0.150 g, 0.58 mmol) and [1,1′-bis(di-tert-butylphosphino)ferrocene]dichloropalladium(II) catalyst (0.038 g, 0.058 mmol) in EtOH (3.0 mL, degassed under nitrogen) was added K₃PO₄ (1M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100% Hexane—2/1 Hexane/EtOAc—100% EtOAc—10% MeOH/EtOAc) and triturated with MeOH and Hexane to afford the title compound as an off-white solid (0.004 g, 0.013 mmol, 3%). ¹H NMR (DMSO-d₆): δ 1.34 (t, J=7.2 Hz, 3H), 4.33 (q, J=7.2 Hz, 2H), 5.59 (br s, 2H), 7.27 (d, J=4.8 Hz, 1H), 7.35 (d, J=2.0 Hz, 1H), 7.41 (d, J=8.8 Hz, 1H), 7.65 (dd, J=1.6, 8.4 Hz, 1H), 8.21 (s, 1H), 8.45 d, J=5.2 Hz, 1H), 11.61 (br s, 1H), 12.59 (br s, 1H). HRMS: For C₁₇H₁₆O₂N₅ requires 322.1299 found 322.1293.

### Example 233

### 3-(2-Amino-4-chloropyridin-3-yl)propiolamide

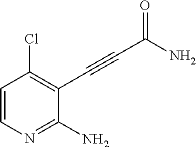 [SMILES: NC(=O)C#Cc1c(Cl)ccnc1N]

A solution of 4-chloro-3-iodopyridin-2-amine (1.00 g, 3.9 mmol), copper (1) iodide (0.38 g, 0.2 mmol) and bis(triphenylphosphine) palladium(II) chloride (0.28 g, 0.4 mmol) in 16 ml of N,N-dimethyformamide-triethylamine (4:1) was degassed in sealed tube followed by addition of propiolamide (1.00 g, 14.5 mmol). The reaction mixture was heated to 80° C. for 10 hours. The resulting mixture was diluted with EtOAc, filtered through silica, concentrated under reduced pressure and filtrated through silica (petroleum ether 60-80%/acetone 7:3) to give the crude product as a brown solid. (0.36 g, 47%) which was used without further purification. ¹H NMR (400 MHz, (CD₃)₂CO): δ 6.31 (br s, 2H), 6.74 (d, J=5.4 Hz, 1H), 7.98 (d, J=5.4 Hz, 1H), 2 protons missing. LRMS: Calculated for C₃H₆ClN₃O 195.0 found 196.0 (M+1).

### 4-(3-amino-1H-indazol-5-yl)-1H-pyrrolo[2,3-b]pyridine-2-carboxamide

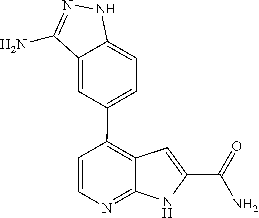 [SMILES: NC(=O)c1cc2c(-c3ccc4[nH]nc(N)c4c3)ccnc2[nH]1]

A solution of 3-(2-amino-4-chloropyridin-3-yl)propiolamide (0.50 g, 2.6 mmol), K₃PO₄ (4.10 g, 19.2 mmol), Sphos (113 mg, 0.26 mmol), bis(triphenylphosphine) palladium(II) chloride (58 mg, 0.26 mmol) and 2-fluoro-5-(4,4,5,5-tetramethyl-1,3,2-dioxaborolan-2-yl)benzonitrile (1.58 g, 6.4 mmol) in 16.6 ml of dioxane-water (5:1) was degassed in sealed tube and submitted to reaction. The reaction mixture was heated to 80° C., o/n. The resulting mixture was diluted with EtOAc, filtered through silica, concentrated under reduced pressure and the residue was filtered through silica (petroleum ether 60-80%/acetone 4:6) to provide a brown solid (0.11 g) which was used as followed without further purification.

To a solution of 3-(2-amino-4-(3-cyano-4-fluorophenyl)pyridin-3-yl)propiolamide (60 mg, 0.22 mmol) in 0.3 ml of EtOH was added hydrazine (0.86 mL, 1 Mol solution in EtOH). The reaction was stirred at 80° C., o/n. The resulting mixture was diluted with EtOAc, washed with water, concentrated under reduced pressure and the residue purified by HPLC to give the product as a yellow solid. (5 mg, 8%)¹H NMR (400 MHz, DMSO-d₆): δ 5.54 (br s, 2H), 7.22 (d, J=4.9 Hz, 1H), 7.40 (d, J=8.6 Hz, 1H), 7.46 (s, 1H), 7.67 (dd, J=8.6, 1.6 Hz, 1H), 8.07 (br s, 1H), 8.20 (d, J=1.6 Hz, 1H), 8.36 (d, J=4.9 Hz, 1H), 11.59 (br s, 1H), 12.12 (br s, 1H). LRMS: Calculated for C₁₅H₁₂N₆O 292.1 found 293.1 (M+1).

### Example 234

### (4-(3-Amino-1H-indazol-5-yl)-1H-pyrrolo[2,3-b]pyridin-2-yl)(pyrrolidin-1-yl)methanone

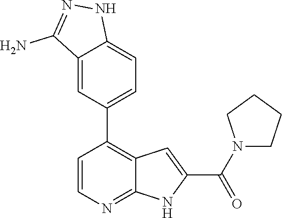 [SMILES: Nc1n[nH]c2ccc(-c3ccnc4[nH]c(C(=O)N5CCCC5)cc34)cc12]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), pyrrolidine (0.5 mL, dried on MS3A) and MeOH (0.1 mL, dried on MS3A), sealed with an aluminium crimp cap fitted with a disposable PTFE/silicon septum, purged with N₂ and heated to 190° C. under microwave irradiation for 90 minutes. The reaction mixture was then evaporated in vacuo and purified by HPLC to afford the title compound as a yellow solid (0.007 g, 0.0122 mmol, 33%). ¹H NMR (400 MHz, DMSO-d₆) δ 1.76-2.05 (m, 4H), 3.55 (t, J=6.6 Hz, 2H), 3.80 (t, J=6.5 Hz, 2H), 7.13 (s, 1H), 7.28 (d, J=4.5 Hz, 1H), 7.49 (d, J=8.8 Hz, 1H), 7.80 (dd, J=8.7, 1.7 Hz, 1H), 8.30 (s, 1H), 8.42 (s, 1H), 12.29 (s, 1H). HRMS: Calculated for C₁₉H₁₉ON₆(M+H⁺)=347.1615; Found: 347.1613

### Example 235

### 4-(3-Amino-1H-indazol-5-yl)-N-cyclopentyl-1H-pyrrolo[2,3-b]pyridine-2-carboxamide

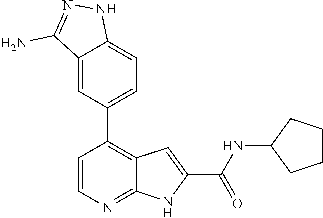 [SMILES: Nc1n[nH]c2ccc(-c3ccnc4[nH]c(C(=O)NC5CCCC5)cc34)cc12]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), cyclopentanamine (0.5 mL, dried on MS3A) and MeOH (0.1 mL, dried on MS3A), sealed with an aluminium crimp cap fitted with a disposable PTFE/silicon septum, purged with N₂ and heated to 190° C. under microwave irradiation for 90 minutes. The reaction mixture was then evaporated in vacuo and purified by HPLC to afford the title compound as a yellow solid (0.0058 g, 0.00986 mmol, 26%). ¹H NMR (400 MHz, DMSO-d₆) δ 1.47-1.59 (m, 4H), 1.63-1.74 (m, 2H), 1.85-1.97 (m, 2H), 4.18-4.31 (m, 1H), 7.24 (d, J=5.0 Hz, 1H), 7.42 (s, 1H), 7.50 (d, J=8.7 Hz, 1H), 7.77 (d, J=8.3 Hz, 1H), 8.26 (s, 1H), 8.36 (d, J=7.2 Hz, 1H), 8.38 (d, J=5.0 Hz, 1H), 12.25 (s, 1H). HRMS: Calculated for C₂₀H₂₁ON₆(M+H⁺)=361.1771; Found: 361.1776

### Example 236

### 4-(3-Amino-1H-indazol-5-yl)-N-cyclohexyl-1H-pyrrolo[2,3-b]pyridine-2-carboxamide

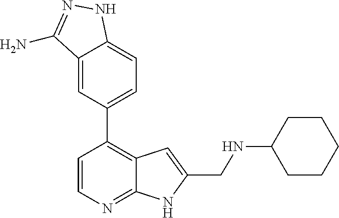 [SMILES: Nc1n[nH]c2ccc(-c3ccnc4[nH]c(CNC5CCCCC5)cc34)cc12]

Ethyl 4-(3-amino-1H-indazol-5-yl)-1H-pyrrolo[2,3-b]pyridine-2-carboxylate (Example 232) (0.060 g, 0.187 mmol), cyclohexylamine (0.5 mL, 4.36 mmol) and a magnetic stirrer bar were added in a 0.5-2 mL Biotage microwave vial which was sealed with an aluminium crimp cap fitted with a disposable PTFE/silicon septum. Then MeOH (0.1 mL) was added and the vial was subjected to microwave irradiation for 90 min at 190° C. After cooling, the solvent was evaporated and the mixture was suspended in 0.1 M NaOH (aquous solution, 5 mL) and extracted with EtOAc (3×8 mL), the organic layer was dried under MgSO₄ and the solvent evaporated in vacuo. The residue was purified by flash chromatography (SiO₂, dichloromethane—dichlorormethane:MeOH 95:5) to obtain the title compound as brown solid (0.010 g, 0.027 mmol, 15%). ¹H NMR (400 MHz, DMSO-d₆) δ 1.25-1.39 (m, 5H), 1.58-1.63 (m, 2H), 1.72-1.78 (m, 2H), 1.82-1.88 (m, 2H), 5.54 (s, 2H), 7.19 (d, J=4.7 Hz, 1H), 7.39-7.42 (m, 2H), 7.66 (d, J=8.4 Hz, 1H), 8.16 (s, 1H), 8.25 (d, J=8.4 Hz, 1H), 8.35 (d, J=4.7 Hz, 1H), 11.58 (s, 1H), 12.09 (s, 1H). HRMS found 375.19 (M+H)⁺ calculated for C₂₁H₂₃ON₆ 375.19

### Example 237

### 4-(3-Amino-1H-indazol-5-yl)-N-isopentyl-1H-pyrrolo[2,3-b]pyridine-2-carboxamide

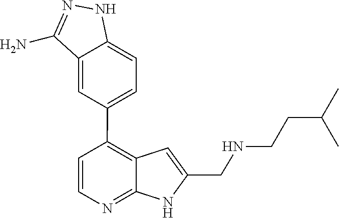 [SMILES: CC(C)CCNCc1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.026 g, 0.0809 mmol, 1 eq.), i-pentylamine (94 μL, 0.810 mmol, 10eq.) EtOH (500 μL, anhydrous). The vial was sealed with an aluminium crimp cap fitted with a disposable PTFE/silicon septum, purged with N₂ and heated to 170° C. under microwave irradiation for 90 minutes. The resulting solution was evaporated to give a yellow solid that was checked by NMR showing the presence of product and starting material. The solid was dissolved with EtOH (300 uL, anhydrous) and i-pentylamine (282 μL, 30 eq.), purged with N₂ and heated to 170° C. under mw irradiation for additional 2 hours. The resulting solution was evaporated to give a yellow solid that was purified by HPLC to afford the title compound as a yellow solid (0.0187 g, 0.0317 mmol, 39%). ¹H NMR (400 MHz, DMSO-d₆) δ 0.92 (d, J=6.6 Hz, 6H), 1.06 (t, J=7.0 Hz, 1H), 1.40-1.49 (m, 2H), 1.61-1.66 (m, 1H), 3.28-3.35 (m, 2H), 7.23 (d, J=5.0 Hz, 1H), 7.38 (s, 1H), 7.45 (d, J=8.5 Hz, 1H), 7.72 (d, J=8.5 Hz, 1H), 8.22 (s, 1H), 8.37 (d, J=4.9 Hz, 1H), 8.43-8.50 (m, 1H), 12.18 (s, 1H). HRMS: Calculated for C₂₀H₂₃ON₆(M+H⁺)=363.1928; Found: 363.1931

### Example 238

### 4-(3-Amino-1H-indazol-5-yl)-N-phenethyl-1H-pyrrolo[2,3-b]pyridine-2-carboxamide

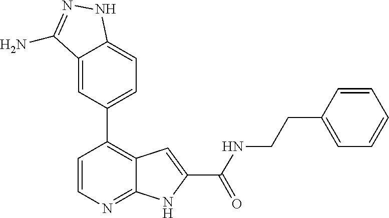 [SMILES: Nc1n[nH]c2ccc(-c3ccnc4[nH]c(C(=O)NCCc5ccccc5)cc34)cc12]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), 2-phenylethanamine (0.183 mL, dried on MS3A) and EtOH (0.367 mL, dried on MS3A), sealed with an aluminium crimp cap fitted with a disposable PTFE/silicon septum, purged with N₂ and heated to 190° C. under microwave irradiation for 90 minutes. The reaction mixture was then evaporated in vacuo and purified by HPLC to afford the title compound as a yellow solid (0.0066 g, 28%). ¹H NMR (400 MHz, DMSO-d₆) δ 2.87 (t, J=7.4 Hz, 2H), 3.52 (app q, J=6.8 Hz, 2H), 7.15-7.34 (m, 6H), 7.39 (s, 1H), 7.46 (d, J=8.6 Hz, 1H), 7.73 (d, J=8.7 Hz, 1H), 8.22 (s, 1H), 8.39 (s, 1H), 8.65 (t, J=5.8 Hz, 1H), 12.22 (s, 1H). HRMS: Calculated for C₂₃H₂₁ON₆(M+H⁺)=397.1771; Found: 397.1766

### Example 239

### 4-(3-Amino-1H-indazol-5-yl)-N-(3-phenylpropyl)-1H-pyrrolo[2,3-b]pyridine-2-carboxamide

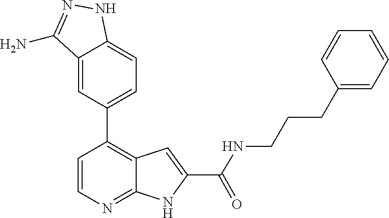 [SMILES: Nc1n[nH]c2ccc(-c3ccnc4[nH]c(C(=O)NCCCc5ccccc5)cc34)cc12]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), 3-phenylpropan-1-amine (0.183 mL, dried on MS3A) and MeOH (0.367 mL, dried on MS3A), sealed with an aluminium crimp cap fitted with a disposable PTFE/silicon septum, purged with N₂ and heated to 160° C. under microwave irradiation for 8 hours. The reaction mixture was then evaporated in vacuo and purified by HPLC to afford the title compound as a yellow solid (0.0059 g, 0.00924 mmol, 25%). ¹H NMR (400 MHz, DMSO-d₆) δ 1.78-1.92 (m, 2H), 2.60-2.70 (m, 2H), 3.31 (app q, J=6.9 Hz, 2H), 7.14-7.34 (m, 6H), 7.40 (s, 1H), 7.47 (d, J=8.7 Hz, 1H), 7.75 (d, J=8.6 Hz, 1H), 8.24 (s, 1H), 8.38 (d, J=4.9 Hz, 1H), 8.56 (t, J=5.4 Hz, 1H), 12.25 (s, 1H). HRMS: Calculated for C₂₄H₂₃ON₆(M+H⁺)=411.1928; Found: 411.1930

### Example 240

### 4-(3-Amino-1H-indazol-5-yl)-N-(2-methoxyethyl)-1H-pyrrolo[2,3-b]pyridine-2-carboxamide

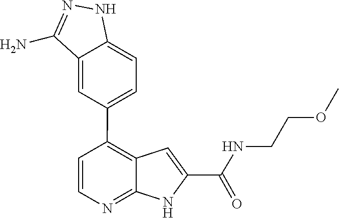 [SMILES: COCCNC(=O)c1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.015 g, 0.0488 mmol), 2-methoxyethanamine (0.5 mL, dried on MS3A) and MeOH (0.1 mL, dried on MS3A), sealed with an aluminium crimp cap fitted with a disposable PTFE/silicon septum, purged with N2 and heated to 190° C. under microwave irradiation for 5 hours. The reaction mixture was then evaporated in vacuo and purified by HPLC to afford the title compound as a yellow solid (0.0106 g, 0.0183 mmol, 38%). ¹H NMR (400 MHz, DMSO-d₆) δ 3.28 (s, 3H), 3.42-3.50 (m, 4H), 7.26 (d, J=5.0 Hz, 1H), 7.46 (s, 1H), 7.51 (d, J=8.7 Hz, 1H), 7.80 (d, J=8.6 Hz, 1H), 8.29 (s, 1H), 8.40 (d, J=5.0 Hz, 1H), 8.61-8.66 (m, 1H), 12.30 (s, 1H). HRMS: Calculated for C₁₈H₁₉O₂N₆(M+H⁺)=351.1564; Found: 351.1567

### Example 241

### 4-(3-Amino-1H-indazol-5-yl)-N-(2-aminoethyl)-1H-pyrrolo[2,3-b]pyridine-2-carboxamide

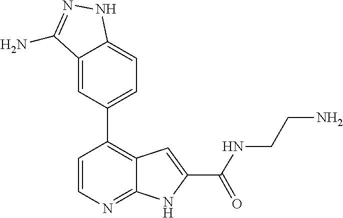 [SMILES: NCCNC(=O)c1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), ethane-1,2-diamine (0.5 mL, dried on MS3A) and MeOH (0.1 mL, dried on MS3A), sealed with an aluminium crimp cap fitted with a disposable PTFE/silicon septum, purged with N2 and heated to 190° C. under microwave irradiation for 90 minutes. The reaction mixture was then evaporated in vacuo and purified by HPLC to afford the title compound as a yellow solid (0.0077 g, 0.0114 mmol, 30%). ¹H NMR (400 MHz, DMSO-d₆) δ 2.99 (app q, J=5.8 Hz, 2H), 3.52 (app q, J=6.0 Hz, 2H), 7.24 (d, J=5.0 Hz, 1H), 7.38 (s, 1H), 7.45 (d, J=8.6 Hz, 1H), 7.71 (d, J=8.6 Hz, 1H), 7.78 (bs, 3H), 8.20 (s, 1H), 8.40 (d, J=5.0 Hz, 1H), 8.68 (t, J=5.7 Hz, 1H), 11.81 (s, OH), 12.27 (s, 1H). HRMS: Calculated for C₁₇H₁₈ON₇ (M+H⁺)=420.2506; Found: 336.1563

### Example 242

### 4-(3-Amino-1H-indazol-5-yl)-N-(2-(dimethylamino)ethyl)-1H-pyrrolo[2,3-b]pyridine-2-carboxamide

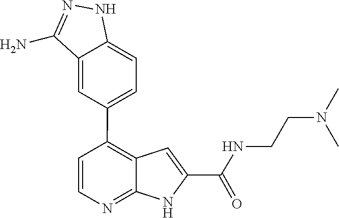 [SMILES: CN(C)CCNC(=O)c1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), N1,N1-dimethylethane-1,2-diamine (0.5 mL, dried on MS3A) and MeOH (0.1 mL, dried on MS3A), sealed with an aluminium crimp cap fitted with a disposable PTFE/silicon septum, purged with N₂ and heated to 190° C. under microwave irradiation for 5 hours. The reaction mixture was then evaporated in vacuo and purified by HPLC to afford the title compound as a yellow solid (0.0075 g, 0.0106, 29%). ¹H NMR (400 MHz, DMSO-d₆) δ 2.85 (d, J=4.6 Hz, 6H), 3.27 (app q, J=5.9 Hz, 2H), 3.64 (app q, J=6.0 Hz, 2H), 7.25 (d, J=5.0 Hz, 1H), 7.40 (s, 1H), 7.46 (d, J=8.7 Hz, 1H), 7.72 (d, J=8.7 Hz, 1H), 8.21 (s, 1H), 8.40 (d, J=4.9 Hz, 1H), 8.77 (t, J=5.8 Hz, 1H), 9.39 (s, 1H), 11.85 (bs, 1H), 12.30 (s, 1H). HRMS: Calculated for C₁₉H₂₂ON₇ (M+H⁺)=364.1880; Found: 364.1880

### Example 243

### (4-(3-Amino-1H-indazol-5-yl)-1H-pyrrolo[2,3-b]pyridin-2-yl)(4-methylpiperazin-1-yl)methanone

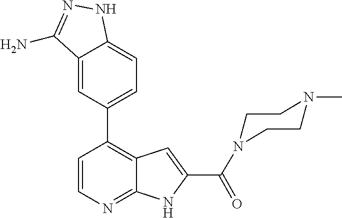 [SMILES: CN1CCN(C(=O)c2cc3c(-c4ccc5[nH]nc(N)c5c4)ccnc3[nH]2)CC1]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), 1-methylpiperazine (0.5 mL, dried on MS3A) and MeOH (0.1 mL, dried on MS3A), sealed with an aluminium crimp cap fitted with a disposable PTFE/silicon septum, purged with N₂ and heated to 200° C. under microwave irradiation for 90 minutes. The reaction mixture was then evaporated in vacuo and purified by HPLC to afford the title compound as a yellow solid (0.0052 g, 0.0725 mmol, 19%). ¹H NMR (400 MHz, DMSO-d₆) δ 2.85 (s, 3H), 3.02-3.15 (m, 2H), 3.29-3.53 (m, 2H), 4.43-4.65 (m, 2H), 7.04 (s, 1H), 7.27 (d, J=4.9 Hz, 1H), 7.41 (d, J=8.7 Hz, 1H), 7.71 (d, J=8.5 Hz, 1H), 8.22 (s, 1H), 8.40 (d, J=4.8 Hz, 1H), 9.82 (bs, 1H), 11.72 (bs, 1H), 12.34 (s, 1H). HRMS: Calculated for C₂₀H₂₂ON₇ (M+H⁺)=376.1880; Found: 376.1876

### Example 244

### 4-(3-Amino-1H-indazol-5-yl)-N-(2-(piperidin-1-yl)ethyl)-1H-pyrrolo[2,3-b]pyridine-2-carboxamide

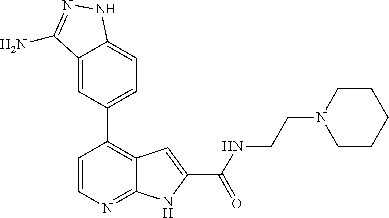 [SMILES: Nc1n[nH]c2ccc(-c3ccnc4[nH]c(C(=O)NCCN5CCCCC5)cc34)cc12]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), 2-(piperidin-1-yl)ethanamine (0.5 mL, dried on MS3A) and MeOH (0.1 mL, dried on MS3A), sealed with an aluminium crimp cap fitted with a disposable PTFE/silicon septum, purged with N₂ and heated to 190° C. under microwave irradiation for 90 minutes. The reaction mixture was then evaporated in vacuo and purified by HPLC to afford the title compound as a yellow solid (0.0061 g, 0.00818, 22%). ¹H NMR (400 MHz, DMSO-d₆) δ 1.31-1.44 (m, 1H), 1.53-1.75 (m, 3H), 1.77-1.89 (m, 2H), 2.87-3.02 (m, 2H), 3.15-3.29 (m, 2H), 7.23 (d, J=4.9 Hz, 1H), 7.38 (d, J=1.7 Hz, 1H), 7.43 (d, J=8.4 Hz, 1H), 7.66-7.71 (m, 1H), 8.18 (s, 1H), 8.39 (d, J=4.9 Hz, 1H), 8.77 (t, J=5.9 Hz, 1H), 9.00 (bs, 1H), 11.73 (s, 1H), 12.30 (s, 1H). HRMS: Calculated for C₂₂H₂₆ON₇ (M+H⁺)=404.2193; Found: 404.2197

### Example 245

### 4-(3-Amino-1H-indazol-5-yl)-N-(2-(butyl(ethyl)amino)ethyl)-1H-pyrrolo[2,3-b]pyridine-2-carboxamide

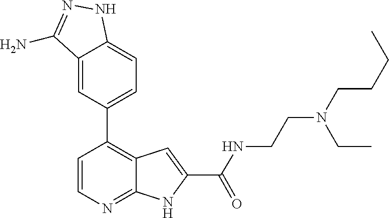 [SMILES: CCCCN(CC)CCNC(=O)c1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), N1-butyl-N1-ethylethane-1,2-diamine (0.183 mL, dried on MS3A) and EtOH (0.367 mL, dried on MS3A), sealed with an aluminium crimp cap fitted with a disposable PTFE/silicon septum, purged with N₂ and heated to 190° C. under microwave irradiation for 90 minutes. The reaction mixture was then evaporated in vacuo and purified by HPLC to afford the title compound as a yellow solid (0.0079 g, 0.0103 mmol, 28%). ¹H NMR (400 MHz, DMSO-d₆) δ 0.90 (t, J=7.4 Hz, 3H), 1.22 (t, J=7.1 Hz, 3H), 1.27-1.39 (m, 2H), 1.50-1.68 (m, 2H), 3.06-3.19 (m, 2H), 3.19-3.34 (m, 4H), 3.64 (app q, J=6.1 Hz, 2H), 7.24 (d, J=4.8 Hz, 1H), 7.37 (s, 1H), 7.43 (d, J=8.6 Hz, 1H), 7.69 (d, J=8.8 Hz, 1H), 8.18 (s, 1H), 8.40 (s, 1H), 8.78 (t, J=5.7 Hz, 1H), 9.17 (bs, 1H), 11.72 (s, 1H), 12.30 (s, 1H). HRMS: Calculated for C₂₃H₃₀ON₇ (M+H⁺)=420.2506; Found: 420.2501

### Example 246

### 4-(3-Amino-1H-indazol-5-yl)-N-(2-(diisopropylamino)ethyl)-1H-pyrrolo[2,3-b]pyridine-2-carboxamide

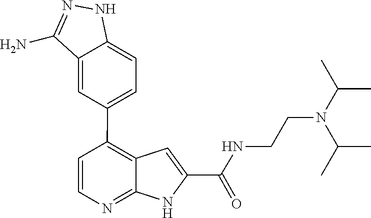 [SMILES: CC(C)N(CCNC(=O)c1cc2c(-c3ccc4[nH]nc(N)c4c3)ccnc2[nH]1)C(C)C]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), N1,N1-diisopropylethane-1,2-diamine (0.183 mL, dried on MS3A) and EtOH (0.367 mL, dried on MS3A), sealed with an aluminium crimp cap fitted with a disposable PTFE/silicon septum, purged with N2 and heated to 190° C. under microwave irradiation for 90 minutes. The reaction mixture was then evaporated in vacuo and purified by HPLC to afford the title compound as a yellow solid (0.0070 g, 0.0092 mmol, 25%). ¹H NMR (400 MHz, DMSO-d₆) δ 1.31 (d, J=6.4 Hz, 12H), 3.16-3.26 (m, 2H), 3.54-3.63 (m, 2H), 3.65-3.77 (m, 2H), 7.24 (d, J=4.9 Hz, 1H), 7.36 (s, 1H), 7.44 (d, J=8.6 Hz, 1H), 7.69 (d, J=8.7 Hz, 1H), 8.18 (s, 1H), 8.40 (d, J=5.0 Hz, 1H), 8.62 (s, 1H), 8.84 (t, J=5.2 Hz, 1H), 11.74 (bs, 1H), 12.33 (s, 1H). HRMS: Calculated for C₂₃H₃₀ON₇ (M+H⁺)=420.2506; Found: 420.2503

### Example 247

### 4-(3-Amino-1H-indazol-5-yl)-N-(3-(dimethylamino)propyl)-1H-pyrrolo[2,3-b]pyridine-2-carboxamide

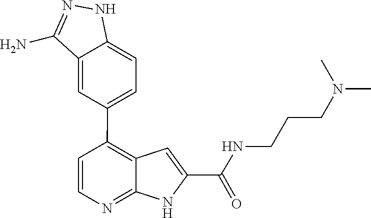 [SMILES: CN(C)CCCNC(=O)c1cc2c(-c3ccc4[nH]nc(N)c4c3)ccnc2[nH]1]

A 0.5-2 mL MW vial was charged with methyl 4-(3-amino-1H-indazol-5-yl)-1H-pyrrolo[2,3-b]pyridine-2-carboxylate (Example 231) (0.012 g, 0.0373 mmol), N1,N1-dimethylpropane-1,3-diamine (0.5 mL, dried on MS3A) and MeOH (0.1 mL, dried on MS3A), sealed with an aluminium crimp cap fitted with a disposable PTFE/silicon septum, purged with N₂ and heated to 190° C. under mw irradiation for 15 minutes. The reaction mixture was then evaporated in vacuo and purified by HPLC to afford the title compound as a yellow solid (0.0071 g, 0.00987 mmol, 26%). ¹H NMR (400 MHz, DMSO-d₆) δ 1.89 (m, 2H), 2.78 (d, J=4.8 Hz, 6H), 3.06-3.16 (m, 2H), 3.36 (app q, J=6.1 Hz, 2H), 7.24 (d, J=4.9 Hz, 1H), 7.39 (d, J=1.3 Hz, 1H), 7.45 (d, J=8.7 Hz, 1H), 7.71 (d, J=8.4 Hz, 1H), 8.21 (s, 1H), 8.39 (d, J=4.8 Hz, 1H), 8.69 (t, J=6.0 Hz, 1H), 9.34 (bs, 1H), 12.23 (s, 1H). HRMS: Calculated for C₂₀H₂₄ON₇ (M+H⁺)=378.2037; Found: 378.2038

### Section 11—Compounds of the Formula

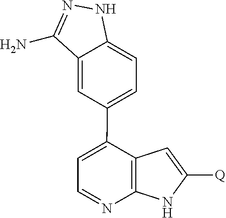

where Q is -L₁-Y₁-L₂-Q₁ and Y₁ is N(R_{y1})_or Q, is heterocyclyl.

### 4-Chloro-1-(methoxymethyl)-7-azaindole-2-carbaldehyde

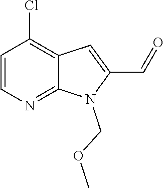

A solution of 4-chloro-1-(methoxymethyl)-7-azaindole (0.66 g, 3.3 mmol) in anhydrous THF (20 ml) was added slowly at −75° C. to freshly prepare lithium diisopropylamide solution (5 mmol, prepared by slowly adding 2M n-butyllithium (2.5 ml) to 0.7 ml diisopropylamine in 20 ml of anhydrous THF). Then, the reaction mixture was aged for 30 min in −75° C. Thereafter, the reaction was quenched by addition of dimethylformamide (0.77 ml, 10 mmol) and stirred for 1 h at −75° C. The mixture was then treated with sulphuric acid (2M, 10 ml), diluted with diethyl ether, and extracted. The separated organic layer was washed with water, dried over magnesium sulfate, and evaporated to afford the desired compound as a yellow solid (0.68 g, 92%), ¹H NMR (400 MHz, DMSO-d₆) δ ppm 3.20 (s, 3H) 5.94 (s, 2H) 7.50 (d, J=4.83 Hz, 1H) 7.67 (s, 1H) 8.54 (d, J=4.83 Hz, 1H) 10.03 (s, 1H). ¹³C NMR (100 MHz, DMSO-d₆) δ ppm 56.65, 73.53, 113.78, 118.27, 118.65, 136.02, 138.49, 149.76, 150.92, 184.57. m/z (ESI-MS) [M−OCH₃]⁺193.0.

### 4-Chloro-7-azaindole-2-carbaldehyde

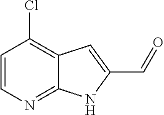 [SMILES: O=Cc1cc2c(Cl)ccnc2[nH]1]

To a cooled solution of 4-chloro-1-(methoxymethyl)-7-azaindole-2-carbaldehyde in anhydrous dichloromethane (5 ml) at 0° C., bromo-catecolborane (0.28 g, 1.3 mmol) was added and the mixture was stirred for 1.5 h. To the thick suspension, potassium acetate (3.2 g, 30 mmol) and ethanol (10 ml) were added and the reaction was stirred for 24 h at room temperature. Then, the solvent was evaporated and the residue was triturated with water for 3 h. The suspension was then filtrated under vacuum and washed with saturated sodium bicarbonate solution to give 4-chloro-7-azaindole-2-carbaldehyde as a creamy solid (0.15 g, 80%), ¹H NMR (400 MHz, DMSO-d₆) δ ppm 7.37 (d, J=4.83 Hz, 1H) 7.49 (d, J=2.20 Hz, 1H) 8.44 (d, J=4.83 Hz, 1H) 9.93 (s, 1H) 12.96 (br. s., 1H). ¹³C NMR (100 MHz, DMSO-d₆) δ ppm 110.39, 117.46, 118.84, 137.06, 138.02, 149.37, 150.54, 184.25. m/z (ESI-MS) [M]-179.0.

### General Procedure a:

The appropriate aldehyde (1 eq.) and required amine (1.3 eq.) were allowed to react in the presence of sodium triacetoxyborohydride (1.5 eq.) and acetic acid (1.3 eq.) in dimethylacetamide (2 ml) at room temperature for 48 h. The reaction mixture then poured into 1M sodium carbonate solution and stirred in ice path for 3 h then filtered. The solid was then collected and purified by column chromatography.

### General Procedure B:

A suspension of the required 4-chloroazaindole substrate (1 eq.), 5-(4,4,5,5-tetramethyl-1,3,2-dioxaborolan-2-yl)-1H-indazol-3-amine (1.2-1.5 eq.) and base (2 eq.) in 1:3 of solvent was deoxygenated with nitrogen in sealed tube. Then [1,1′-bis(di-tert-butylphosphino)ferrocene]dichloropalladium(II) catalyst (0.05 eq.) was added then the tube was sealed and the mixture allowed to stirred at 90-100° C. for 18 h. After the reaction was cooled to room temperature, EtOAc and water were added. Extracted organic layer was dried over magnesium sulfate and concentrated under reduced pressure and the residue purified by column chromatography.

### Example 248

### N-((4-Chloro-7-azaindole)methyl)-N-(tert-butyl)amine

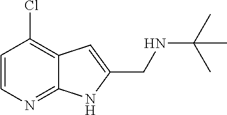 [SMILES: CC(C)(C)NCc1cc2c(Cl)ccnc2[nH]1]

4-Chloro-7-azaindole-2-carbaldehyde (0.08 g, 0.44 mmol) was reacted with tert-butylamine (0.06 ml, 0.57 mmol), sodium triacetoxyborohydride (0.14 g, 0.66 mmol) and acetic acid (0.03 ml, 0.66 mmol) as described in general procedure A to afford the desired compound as a yellow solid (90 mg, 86%), ¹H NMR (400 MHz, DMSO-d₆) δ ppm 1.09 (s, 9H) 3.82 (s, 2H) 6.35 (s, 1H) 7.12 (d, J=4.83 Hz, 1H) 8.07 (d, J=4.83 Hz, 1H). ¹³C NMR (100 MHz, DMSO-d₆) b ppm 29.33, 49.15, 50.79, 95.51, 115.63, 119.69, 133.22, 142.61, 143.58, 149.72. m/z (ESI-MS) [M−tBu-amine]⁺ 165.1 (100%) [M]⁺ 238.1 (20%)

### 5-(2-((tert-Butylamino)methyl)-1H-pyrrolo[2,3-b]pyridine-4-yl)-1H-indazol-3-amine

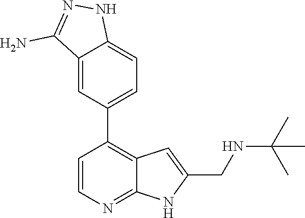 [SMILES: CC(C)(C)NCc1cc2c(-c3ccc4[nH]nc(N)c4c3)ccnc2[nH]1]

N-((4-Chloro-7-azaindole)methyl)-N-(tert-butyl)amine (0.034 g, 0.14 mmol), 5-(4,4,5,5-tetramethyl-1,3,2-dioxaborolan-2-yl)-1H-indazol-3-amine (0.055 g, 0.215 mmol), 1M potassium phosphate solution (0.28 ml, 0.28 mmol), [1,1′-bis(di-tert-butylphosphino)ferrocene]dichloropalladium(II) catalyst (0.004 g, 0.007 mmol) in 0.84 ml ethanol were reacted as described in general procedure B and chromatographic purification (0-10% methanol in EtOAc and 1% triethylamine), gave the titled compound as a white solid (33 mg, 70%). ¹H NMR (400 MHz, DMSO-d₆) δ ppm 1.10 (s, 9H) 3.85 (s, 2H) 5.51 (s, 2H) 6.55 (s, 1H) 7.12 (d, J=4.83 Hz, 1H) 7.38 (d, J=8.79 Hz, 1H) 7.65 (d, J=8.79, 1H) 8.14 (s, 1H) 8.16 (d, J=5.27 Hz, 1H) 11.54 (s, 1H). ¹³C NMR (100 MHz, DMSO-d₆) 29.16, 48.94, 50.77, 96.52, 110.24, 114.38, 115.05, 118.30, 120.63, 127.12, 128.57, 139.01, 141.29, 141.63, 142.46, 149.83, 150.01. m/z (ESI-HRMS) calculated for C₁₉H₂₃N₆=335.1979 found=335.1981.

### Example 249

### N-((4-Chloro-7-azaindole)methyl)-N-isopentylamine

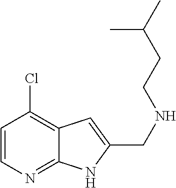 [SMILES: CC(C)CCNCc1cc2c(Cl)ccnc2[nH]1]

4-Chloro-7-azaindole-2-carbaldehyde (0.12 g, 0.66 mmol) was reacted with isopentylamine (0.1 ml, 0.86 mmol), sodium triacetoxyborohydride (0.22 g, 0.99 mmol) and acetic acid (0.06 ml, 0.99 mmol) as described in general procedure A to afforded the desired compound as brown solid (125.6 mg, 75%). ¹H NMR (400 MHz, DMSO-d₆) δ ppm 0.84 (d, J=6.15 Hz, 6H) 1.21-1.38 (m, 2H) 1.53-1.66 (m, 1H) 3.82 (s, 2H) 6.35 (s, 1H) 7.13 (d, J=4.83 Hz, 1H) 8.08 (d, J=4.39 Hz, 1H). ¹³C NMR (100 MHz, DMSO-d₆) δ ppm 23.20, 26.02, 39.20, 46.77, 47.24, 96.17, 115.70, 119.66, 133.33, 142.39, 142.79, 149.78 m/z (ESI-MS) [M]⁺ 252.1.

### 5-(2-((Isopentylamino)methyl)-1H-pyrrolo[2,3-b]pyridine-4-yl)-1H-indazol-3-amine

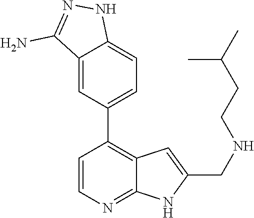 [SMILES: CC(C)CCNCc1cc2c(-c3ccc4[nH]nc(N)c4c3)ccnc2[nH]1]

N-((4-Chloro-7-azaindole)methyl)-N-isopentylamine (0.08 g, 0.32 mmol), 5-(4,4,5,5-tetramethyl-1,3,2-dioxaborolan-2-yl)-1H-indazol-3-amine (0.12 g, 0.48 mmol), 1M potassium phosphate solution (0.64 ml, 0.64 mmol), [1,1′-bis(di-tert-butylphosphino)ferrocene]dichloropalladium(II) catalyst (0.01 g, 0.016 mmol) in 1.9 ml ethanol were reacted as described in general procedure B and chromatographic purification (0-15% methanol in EtOAc and 1% triethylamine), gave the titled compound as reddish solid (47.1 mg, 42%), ¹H NMR (400 MHz, DMSO-d₆) δ ppm 0.83 (d, J=56.59 Hz, 6H) 1.32 (q, J=7.03 Hz, 2H) 1.61 (m, 1H) 3.38 (t, J=7.03 Hz, 2H) 3.85 (s, 2H) 5.51 (s, 2H) 6.56 (s, 1H) 7.13 (d, J=5.27 Hz, 1H) 7.37 (d, J=8.35 Hz, 1H) 7.63-7.67 (m, 1H) 8.13-8.15 (m, 1H) 8.17 (d, J=5.27 Hz, 1H) 11.54 (br. s, 1H). ¹³C NMR (100 MHz, DMSO-d₆) 23.21, 26.04, 39.22, 47.03, 47.26, 94.88, 110.33, 114.46, 115.14, 118.33, 120.74, 127.20, 128.62, 140.78, 140.91, 141.64, 142.57, 150.02, 150.37. m/z (ESI-HRMS) calculated for C₂₀H₂₅N₆=349.2135 found=349.2132.

### Example 250

### tert-Butyl 2-((2-amino-4-chloropyridin-3-yl)ethynyl)piperidine-1-carboxylate

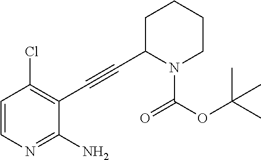 [SMILES: CC(C)(C)OC(=O)N1CCCCC1C#Cc1c(Cl)ccnc1N]

To a solution of 4-chloro-3-iodo-pyridine-2-amine (0.064 g, 0.255 mmol), copper (1) iodide (0.002 g, 0.012 mmol) and bis(triphenylphosphine) palladium(II) chloride (0.008 g, 0.012 mmol) in 2 ml of N,N-dimethyformamide-triethylamine (1:4) was degassed in sealed tube followed by tert-butyl 2-ethynylpiperidine-1-carboxylate (0.064 g, 0.3 mmol). The reaction mixture was stirred at 80° C. for 4 h. After reaction completed, the reaction mixture was diluted with EtOAc and extract with 1M sodium carbonate. Extracted organic layer was washed with brine and dried over magnesium sulfate and concentrated under reduced pressure and the residue purified by column chromatography (80% EtOAc and 1% triethylamine in petroleum ether 60-80%) to give the product as yellow oil (0.042 g, 40%), ¹H NMR (400 MHz, DMSO-d₆) δ ppm 1.39 (s, 9H) 1.40 (d, J=5.19 Hz, 1H) 1.41-1.45 (m, 4H) 2.42 (m, 4H) 6.65 (br. s., 2H) 7.18 (d, J=5.49 Hz, 1H) 8.12 (d, J=5.49 Hz, 1H). ¹³C NMR (100 MHz, DMSO-d₆) δ ppm 19.81, 25.16, 28.29, 28.50, 40.27, 66.82, 70.27, 95.83, 115.87, 119.57, 133.31, 141.73, 143.04, 152.64, 169.32. m/z (ESI-MS) [M]⁺ 336.1.

### Tert-butyl 2-(4-chloro-7-azaindol-2-yl)piperdine-1-carboxylate

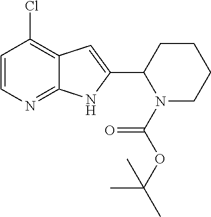 [SMILES: CC(C)(C)OC(=O)N1CCCCC1c1cc2c(Cl)ccnc2[nH]1]

To a solution of tert-butyl 2-((2-amino-4-chloropyridin-3-yl)ethynyl)piperidine-1-carboxylate (0.04 g, 0.12 mmol) in 1,4 dioxane (1 ml), potassium tert-butoxide (0.033 g, 0.3 mmol) and 18-crown-6 (0.003 g, 10%, 0.012 mmol) were added and the mixture was stirred at 110° C. for 18h. After the reaction was cooled to room temperature, EtOAc and 1 M sodium carbonate solution were added. Extracted organic layer was dried over magnesium sulfate and concentrated under reduced pressure to give the product as yellow solid (38.8 mg, 96%), ¹H NMR (400 MHz, DMSO-d₆) δ ppm 1.41 (s, 9H) 1.51-1.64 (m, 3H) 1.75 (d, J=3.08 Hz, 1H) 1.81 (d, J=1.32 Hz, 1H) 1.30-2.40 (m, 1H) 2.86-2.99 (m, 1H) 3.96 (d, J=12.74 Hz, 1H) 5.41-5.46 (m, 1H) 6.14 (s, 1H) 7.17 (d, J=5.27 Hz, 1H) 8.12 (d, J=5.27 Hz, 1H) 11.98 (br. s., 1H). ¹³C NMR (100 MHz, DMSO-d₆) δ ppm 19.81, 25.16, 28.29, 28.50, 40.27, 66.82, 70.27, 95.83, 115.87, 119.57, 133.31, 141.73, 143.04, 152.64, 169.32. m/z (ESI-MS) [M]⁺ 336.1.

### 4-Chloro-2-(piperdin-2yl)-7-azaindole

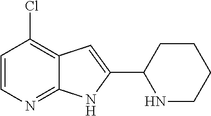 [SMILES: Clc1ccnc2[nH]c(C3CCCCN3)cc12]

Trifloroacetic acid (0.03 ml) was added to a solution of tert-butyl 2-(4-chloro-7-azaindol-2-yl)piperdine-1-carboxylate (0.03 g, 0.09 mmol) in dicloromethane (1 ml) and the reaction was stirred at room temperature for 3 h. The reaction mixture was diluted with EtOAc, washed with 1 M sodium carbonate, dried over magnesium sulfate and concentrated under vacuum under reduced pressure and the residue purified by column chromatography (80% EtOAc and 1% triethylamine in petroleum ether 60-80%) to give the product as a yellow solid (18 mg, 85%). ¹H NMR (400 MHz, DMSO-d₆) δ ppm 1.58-1.72 (m, 3H) 1.78-1.95 (m, 4H) 2.22 (d, J=14.94 Hz, 1H) 4.11 (q, J=5.27 Hz, 1H) 4.44 (dd, J=10.33, 1.10 Hz, 1H) 6.62 (s, 1H) 7.26 (d, J=5.27 Hz, 1H) 8.22 (d, J=5.27 Hz, 1H) 12.20 (br. s., 1H). ¹³C NMR (100 MHz, DMSO-d₆) δ ppm 19.81, 25.16, 28.29, 77.21, 79.60, 95.83, 115.87, 119.57, 113.31, 143.04, 143.03, 154.91. m/z (ESI-MS) [M]⁺ 236.1.

### 5-(2-(Piperidin-2-yl)-1H-pyrrolo[2,3-b]pyridine-4-yl)-1H-indazol-3-amine

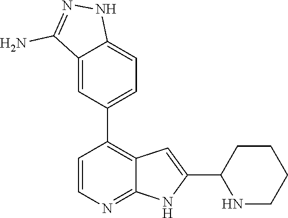 [SMILES: Nc1n[nH]c2ccc(-c3ccnc4[nH]c(C5CCCCN5)cc34)cc12]

4-Chloro-2-(piperidin-2-yl)-7-azaindole (0.03 g, 0.12 mmol), 5-(4,4,5,5-tetramethyl-1,3,2-dioxaborolan-2-yl)-1H-indazol-3-amine (0.05 g, 0.19 mmol), 1M potassium phosphate solution (0.25 ml, 0.25 mmol), [1,1′-bis(di-tert-butylphosphino)ferrocene]dichloropalladium(II) catalyst (0.0016 g, 0.006 mmol) in 0.7 ml ethanol were reacted as described in general procedure B and chromatographic purification (90% EtOAc and 1% triethylamine in petroleum ether 60-80%), gave the titled compound as white solid (10.5 mg, 79%). ¹H NMR (400 MHz, DMSO-d₆) b ppm 1.41-1.52 (m, 2H) 1.59 (d, J=11.86 Hz, 2H) 1.79-1.89 (m, 1H) 1.99 (d, J=10.55 Hz, 1H) 2.66-2.75 (m, 1H) 3.07 (d, J=11.86 Hz, 1H) 3.80-3.89 (m, 1H) 5.52 (s, 2H) 6.55 (s, 1H) 7.13 (d, J=4.83 Hz, 1H) 7.38 (d, J=8.79 Hz, 1H) 7.65 (dd, J=8.57, 1.54 Hz, 1H) 8.13 (s, 1H) 8.18 (d, J=5.27 Hz, 1H) 11.54 (br. s., 1H) 11.58 (br. s., 1H). ¹³C NMR (100 MHz, DMSO-d₆) δ ppm 24.57, 25.59, 32.38, 46.66, 55.04, 95.97, 110.24, 114.43, 115.07, 117.98, 120.63, 127.09, 128.47, 141.13, 141.58, 142.75, 142.91, 149.71, 150.26. m/z (ESI-HRMS) calculated for C₁₉H₂₁N₆=333.1822 found=333.1820.

### Example 251

### N-((4-Chloro-7-azaindole)methyl)-N-cyclohexylamine

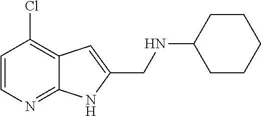 [SMILES: Clc1ccnc2[nH]c(CNC3CCCCC3)cc12]

4-Chloro-7-azaindole-2-carbaldehyde (0.1 g, 0.55 mmol) was reacted with cyclohexylamine (0.1 ml, 0.72 mmol), sodium triacetoxyborohydride (0.17 g, 0.82 mmol) and acetic acid (0.04 ml, 0.72 mmol) as described in general procedure A and chromatographic purification (30-100% EtOAc in petroleum ether 60-80%) afforded the desired compound as a yellow solid (40.9 mg, 41%). ¹H NMR (400 MHz, DMSO-d₆) δ ppm 0.98-1.10 (m, 2H) 1.14 (m, 2H) 1.53 (m, 2H) 1.61-1.71 (m, 2H) 1.81 (m, 2H) 2.30-2.39 (m, 1H) 3.87 (s, 2H) 6.35 (s, 1H) 7.12 (d, J=4.83 Hz, 1H) 8.07 (d, J=5.27 Hz, 1H) 11.91 (br. s., 1H). ¹³C NMR (100 MHz, DMSO-d₆) δ ppm 24.87, 26.44, 33.29, 43.62, 55.40, 96.06, 115.70, 119.67, 120.07, 130.97, 142.80, 150.56. m/z (ESI-MS) [M−cyclohexylamine]⁺ 165.1 (100%) [M]⁺ 264.2 (40%).

### 5-(2-((cyclohexylamino)methyl)-1H-pyrrolo[2,3-b]339yridine-4-yl)-1H-indazol-3-amine

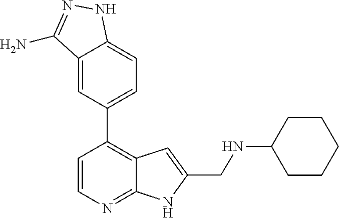 [SMILES: Nc1n[nH]c2ccc(-c3ccnc4[nH]c(CNC5CCCCC5)cc34)cc12]

N-((4-Chloro-7-azaindole)methyl)-N-cyclohexylamine (0.04 g, 0.15 mmol), 5-(4,4,5,5-tetramethyl-1,3,2-dioxaborolan-2-yl)-1H-indazol-3-amine (0.06 g, 0.22 mmol), 1M potassium phosphate solution (0.3 ml, 0.3 mmol), [1,1′-bis(di-tert-butylphosphino)ferrocene]dichloropalladium(II) catalyst (0.005 g, 0.007 mmol) in 0.9 ml ethanol were reacted as described in general procedure B and chromatographic purification (0-18% methanol in EtOAc and 1% triethylamine), gave the titled compound as brown solid (26.6 mg, 49%). ¹H NMR (400 MHz, DMSO-d₆) δ ppm 1.00-1.09 (m, 2H) 1.15-1.21 (m, 2H) 1.47-1.55 (m, 2H) 1.58-1.69 (m, 2H) 1.83 (m, 2H) 2.37 (m, 1H) 3.89 (s, 2H) 5.51 (s, 2H) 6.55 (s, 1H) 7.13 (d, J=5.27 Hz, 1H) 7.37 (d, J=8.79 Hz, 1H) 7.65 (d, J=7.03 Hz, 1H) 8.14 (s, 1H) 8.17 (d, J=4.83 Hz, 1H) 11.54 (br. s, 1H). ¹³C NMR (100 MHz, DMSO-d₆) 24.77, 26.31, 33.09, 43.74, 55.41, 97.78, 110.27, 111.54, 114.40, 118.26, 120.66, 127.12, 128.52, 135.65, 140.90, 142.19, 142.50, 149.90, 150.28. m/z (ESI-HRMS) calculated for C₂₁H₂₅N₆=361.2135 found=361.2138.

### Example 252

### N-((4-Chloro-7-azaindole)methyl)-aniline

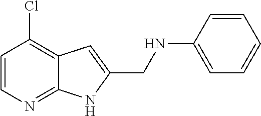 [SMILES: Clc1ccnc2[nH]c(CNc3ccccc3)cc12]

4-Chloro-7-azaindole-2-carbaldehyde (0.12 g, 0.66 mmol) was reacted with aniline (0.08 ml, 0.86 mmol), sodium triacetoxyborohydride (0.21 g, 0.99 mmol) and acetic acid (0.06 ml, 0.99 mmol) as described in general procedure A to afford the desired compound as a white solid (0.07 g, 41%), (LC-MS purity=93%). ¹H NMR (400 MHz, CDCl₃) δ 5 4.43 (d, J=5.71 Hz, 2H) 6.53 (t, J=7.03 Hz, 1H) 6.65 (d, J=7.91 Hz, 1H) 7.07 (t, J=7.47 Hz, 1H) 7.11-7.17 (m, 1H) 7.26-7.34 (m, 2H) 7.42-7.50 (m, 1H) 8.12 (s, 1H) 8.09 (d, J=5.27 Hz, 1H) 11.55 (s, 1H) 11.67 (s, 1H). ¹³C NMR (100 MHz, CDCl₃) δ ppm 41.05, 96.11, 113.06, 115.83, 116.89, 121.59, 129.44, 133.43, 141.36, 142.97, 146.86, 148.91. m/z (ESI-MS) [M]⁻ 256.1.

### 5-(2-((Phenylamino)methyl)-1H-pyrrolo[2,3-b]340yridine-4-yl)-1H-indazol-3-amine

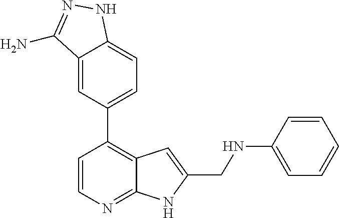 [SMILES: Nc1n[nH]c2ccc(-c3ccnc4[nH]c(CNc5ccccc5)cc34)cc12]

N-((4-Chloro-7-azaindole)methyl)-aniline 0.039 g, 0.15 mmol), 5-(4,4,5,5-tetramethyl-1,3,2-dioxaborolan-2-yl)-1H-indazol-3-amine (0.059 g, 0.22 mmol), 1M potassium phosphate solution (0.3 ml, 0.3 mmol), [1,1′-bis(di-tert-butylphosphino)ferrocene]dichloropalladium(II) catalyst (0.0018 g, 0.007 mmol) in 0.9 ml ethanol were reacted as described in general procedure B and chromatographic purification (80% EtOAc and 1% triethylamine in petroleum ether 60-80%), gave the titled compound as white solid (32 mg, 60%). ¹H NMR (400 MHz, DMSO-d₆) δ 4.42 (d, J=5.71 Hz, 2H) 5.50 (s, 2H) 6.03 (t, J=5.93 Hz, 1H) 6.53 (t, J=7.69 Hz, 1H) 6.59-6.69 (m, 3H) 7.01-7.08 (m, 2H) 7.13 (d, J=5.27 Hz, 1H) 7.36 (d, J=8.35 Hz, 1H) 7.62 (d, J=8.79 Hz, 1H) 8.12 (s, 1H) 8.18 (d, J=5.27 Hz, 1H) 11.55 (s, 1H) 11.67 (s, 1H). ¹³C NMR (100 MHz, DMSO-d₆) 41.41, 97.89, 110.34, 113.11, 114.60, 115.16, 116.80, 118.42, 120.75, 127.16, 128.56, 129.38, 139.63, 141.09, 141.63, 142.77, 149.03, 149.94, 150.35. m/z (ESI-HRMS) calculated for C₂₁H₁₉N₆=355.1666 found=355.1665.

### Example 253

### 2-(Benzyloxy)-N-((4-chloro-1-(methoxymethyl)-7-azaindol-2-yl)methyl)aniline

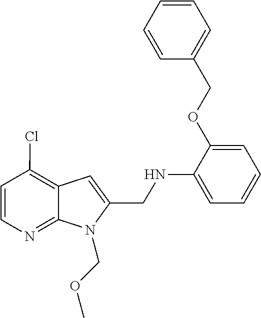 [SMILES: COCn1c(CNc2ccccc2OCc2ccccc2)cc2c(Cl)ccnc21]

4-Chloro-1-(methoxymethyl)-7-azaindole-2-carbaldehyde (0.3 g, 1.3 mmol) was reacted 2-(benzoxy)aniline (0.34 ml, 1.7 mmol), sodium triacetoxyborohydride (0.41 g, 1.95 mmol) and acetic acid (0.11 ml, 1.95 mmol) as described in general procedure A to afforded the desired compound as a white solid (209 mg, 40%). ¹H NMR (400 MHz, DMSO-d₆) δ ppm 3.18 (s, 3H) 4.65 (d, J=6.10 Hz, 2H) 5.17 (s, 2H) 5.64 (t, J=6.18 Hz, 1H) 5.74 (s, 2H) 6.42 (s, 1H) 6.52-6.58 (m, 1H) 6.64 (d, J=7.78 Hz, 1H) 6.74 (t, J=7.63 Hz, 1H) 6.94 (d, J=8.09 Hz, 1H) 7.25 (d, J=5.19 Hz, 1H) 7.30-7.35 (m, 1H) 7.40 (t, J=7.40 Hz, 2H) 7.53 (d, J=5.32 Hz, 2H) 8.20 (d, J=5.19 Hz, 1H). ¹³C NMR (100 MHz, DMSO-d₆) δ ppm 56.31, 63.44, 69.96, 72.67, 97.60, 110.75, 112.22, 116.74, 116.99, 119.01, 121.80, 128.08, 128.93, 134.02, 137.95, 138.22, 141.99, 143.38, 146.05, 150.01. m/z (ESI-MS) [M−OCH₃]⁺ 376.0

### 5-(2-(((2-(Benzyloxy)phenyl)amino)methyl)-1-(methoxymethyl)-7-azainol-4-yl)-1H-indazol-3-amine

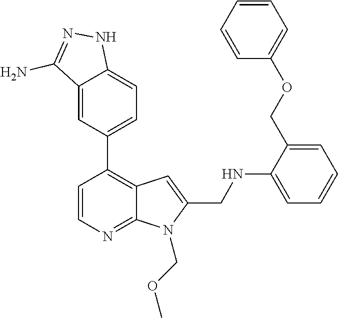 [SMILES: COCn1c(CNc2ccccc2COc2ccccc2)cc2c(-c3ccc4[nH]nc(N)c4c3)ccnc21]

2-(Benzyloxy)-N-((4-chloro-1-(methoxymethyl)-7-azaindol-2-yl)methyl)aniline (0.05 g, 0.12 mmol) 5-(4,4,5,5-tetramethyl-1,3,2-dioxaborolan-2-yl)-1H-indazol-3-amine (0.05 g, 0.19 mmol), 1M potassium phosphate solution (0.24 ml, 0.24 mmol), [1,1′-bis(di-tert-butylphosphino)ferrocene]dichloropalladium(II) catalyst (0.004 g, 0.006 mmol) in 0.72 ml ethanol were reacted as described in general procedure and chromatographic purification (60-100% EtOAc in petroleum ether 60-80%), gave the titled compound as white solid (55.6 mg, 91%). ¹H NMR (400 MHz, DMSO-d₆) δ ppm 3.19 (s, 3H) 4.64 (d, J=5.71 Hz, 2H) 5.12 (s, 2H) 5.64-5.57 (m, 3H) 5.77 (s, 2H) 6.48-6.55 (m, 1H) 6.69 (s, 2H) 6.71 (d, J=7.47 Hz, 1H) 6.88 (d, J=7.47 Hz, 1H) 7.22 (d, J=4.83 Hz, 1H) 7.24-7.37 (m, 4H) 7.47 (d, J=7.03 Hz, 2H) 7.58 (dd, J=8.57, 1.54 Hz, 1H) 8.10 (s, 1H) 8.28 (d, J=4.83 Hz, 1H) 11.57 (s, 1H). ¹³C NMR (100 MHz, DMSO-d₆) δ ppm 56.21, 60.32, 69.97, 72.35, 99.77, 110.44, 110.85, 112.06, 115.17, 115.89, 116.78, 117.70, 120.99, 121.77, 127.09, 128.05, 128.25, 128.91, 137.88, 138.35, 140.04, 141.66 141.89, 143.16, 146.11, 150.22, 150.39. m/z (ESI-MS) [M]⁺ 505.1

### 5-(2-(((2-(Benzyloxy)phenyl)amino)methyl)-1H-pyrrolo[2,3-b]342yridine-4-yl)-1H-indazol-3-amine

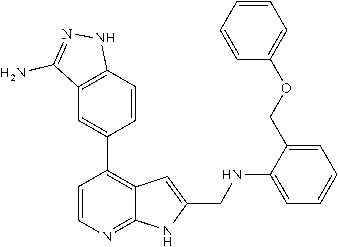 [SMILES: Nc1n[nH]c2ccc(-c3ccnc4[nH]c(CNc5ccccc5COc5ccccc5)cc34)cc12]

To a cool solution of 5-(2-(((2-(Benzyloxy)phenyl)amino)methyl)-1-(methoxymethyl)-7-azainol-4-yl)-1H-indazol-3-amine (0.1 g, 0.25 mmol) in anhydrous dichloromethane (5 ml), a solution of bromo-catecolborane (0.065 g, 0.33 mmol) in anhydrous dichloromethane (5 ml) was added and the mixture was stirred for 1.5h. To the thick suspension, potassium acetate (0.73 g, 7.5 mmol) and ethanol (10 ml) were added and the reaction allowed to stir for 24 h at room temperature. Then the solvent was evaporated and residue triturated with water for 30 min. The suspension then filtered under vacuum and washed with saturated sodium bicarbonate solution. The filtrate then purified using column chromatography (30% EtOAc in petroleum ether 60-80%) to give the product as a yellow solid (40.1 mg, 34%). ¹H NMR (400 MHz, DMSO-d₆) δ ppm 4.14 (s, 2H) 4.30 (s, 2H) 5.45 (t, J=6.18 Hz, 1H) 5.51 (br. s, 2H) 6.57 (s, 1H) 6.59-6.70 (m, 2H) 6.75-6.79 (m, 1H) 6.80-6.88 (m, 1H) 6.97 (d, J=6.96 Hz, 1H) 7.14 (d, J=5.13 Hz, 1H) 7.21-7.23 (m, 4H) 7.34-7.39 (m, 1H) 7.62 (dd, J=8.79, 1.46 Hz, 1H) 8.12 (s, 1H) 8.20 (d, J=5.13 Hz, 1H) 11.83 (s, 1H). ¹³C NMR (100 MHz, DMSO-d₆) δ ppm 48.69, 74.94, 95.95, 99.51, 112.39, 115.34, 118.62, 119.40, 126.53, 126.71, 128.01, 128.57, 128.63, 128.83, 132.79, 136.53, 138.74, 140.94, 141.58, 142.34, 144.21, 147.61, 149.44, 153.13, 161.00. m/z (ESI-MS) [M]⁺ 461.1

### Example 254

### N-((4-Chloro-7-azaindole)methyl)-N-(2-methoxyethyl)amine

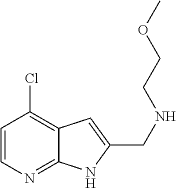 [SMILES: COCCNCc1cc2c(Cl)ccnc2[nH]1]

4-Chloro-7-azaindole-2-carbaldehyde (0.12 g, 0.66 mmol) was reacted with 2-methoxyethylamine (0.07 ml, 0.86 mmol), sodium triacetoxyborohydride (0.22 g, 0.99 mmol) and acetic acid (0.06 ml, 0.99 mmol) as described in general procedure A to afforded the desired compound as yellow oil (55 mg, 34%). ¹H NMR (400 MHz, DMSO-d₆) δ ppm 2.65 (t, J=5.49 Hz, 2H) 3.23 (s, 3H) 3.40 (t, J=5.49 Hz, 2H) 3.85 (s, 2H) 6.35 (s, 1H) 7.13 (d, J=5.27 Hz, 1H) 8.09 (d, J=5.27 Hz, 1H) 11.87 (br. S, 1H). ¹³C NMR (100 MHz, DMSO-d₆) b ppm 46.60, 48.31, 58.55, 72.28, 96.28, 115.73, 119.63, 133.39, 142.12, 142.86, 149.80 m/z (ESI-MS) [M]⁺ 240.1.

### 5-(2-(((2-Methoxyethyl)amino)methyl)-1H-pyrrolo[2,3-b]343yridine-4-yl)-1H-indazol-3-amine

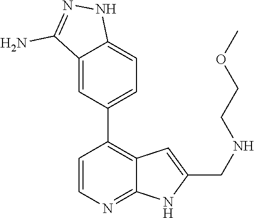 [SMILES: COCCNCc1cc2c(-c3ccc4[nH]nc(N)c4c3)ccnc2[nH]1]

N-((4-Chloro-7-azaindole)methyl)-N-(2-methoxyethyl)amine (0.025 g, 0.1 mmol), 5-(4,4,5,5-tetramethyl-1,3,2-dioxaborolan-2-yl)-1H-indazol-3-amine (0.04 g, 0.15 mmol), 1M potassium phosphate solution (0.2 ml, 0.2 mmol), [1,1′-bis(di-tert-butylphosphino)ferrocene]dichloropalladium(II) catalyst (0.001 g, 0.005 mmol) in 0.6 ml ethanol were reacted as described in general procedure B and chromatographic purification (0-18% methanol in EtOAc and 1% triethylamine), gave the titled compound as yellow solid (22.1 mg, 65%). ¹H NMR (400 MHz, DMSO-d₆) δ ppm 2.67 (t, J=5.71 Hz, 2H) 3.22 (s, 3H) 3.40 (t, J=5.71 Hz, 2H) 3.87 (s, 2H) 5.51 (s, 2H) 6.56 (s, 1H) 7.13 (d, J=5.27 Hz, 1H) 7.37 (d, J=8.79 Hz, 1H) 7.65 (dd, J=8.79 Hz, 1H) 8.14 (s, 1H) 8.17 (d, J=5.27 Hz, 1H) 11.50-11.59 (m, 2H). ¹³C NMR (100 MHz, DMSO-d₆) 46.86, 48.33, 58.54, 72.26, 98.00, 110.35, 114.49, 115.14, 118.30, 120.75, 127.22, 128.11, 138.84, 140.98, 141.91, 142.65, 150.04, 150.37. m/z (ESI-HRMS) calculated for C₁₈H₂₁ON₆=337.1771 found=337.1769.

### Example 255

### N¹-((4-Chloro-7-azaindole)methyl)-N²,N²-dimethyl-1,2-ethanediamine

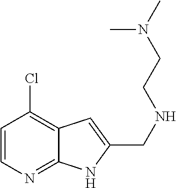 [SMILES: CN(C)CCNCc1cc2c(Cl)ccnc2[nH]1]

4-Chloro-7-azaindole-2-carbaldehyde (0.12 g, 0.66 mmol) was reacted with N¹,N¹dimethyl-1,2ethanediamine (0.09 ml, 0.86 mmol), sodium triacetoxyborohydride (0.22 g, 0.99 mmol) and acetic acid (0.06 ml, 0.99 mmol) as described in general procedure A and chromatographic purification (18% methanol in EtOAc and 1% triethylamine to afforded the desired compound as yellow solid (30.7 mg, 18%). ¹H NMR (400 MHz, DMSO-d₆) δ ppm 2.11 (s, 6H) 2.32 (t, J=6.27 Hz, 2H) 2.59 (t, J=6.40 Hz, 2H) 2.86 (s, 2H) 6.35 (s, 1H) 7.13 (d, J=5.02 Hz, 1H) 8.09 (d, J=5.02 Hz, 1H). m/z (ESI-MS) [M]⁺ 253.1.

### N¹-((4-(3-Amino-1H-indazol-5-yl)-1H-pyrrolo[2,3-b]pyridin-2-yl)methyl)-N2,N2-dimethylethane-1,2-diamine

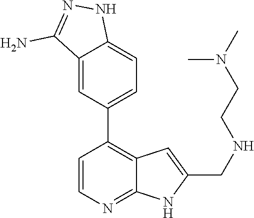 [SMILES: CN(C)CCNCc1cc2c(-c3ccc4[nH]nc(N)c4c3)ccnc2[nH]1]

N¹-((4-Chloro-7-azaindole)methyl)-N²,N²-dimethyl-1,2-ethanediamine (0.03 g, 0.12 mmol), 5-(4,4,5,5-tetramethyl-1,3,2-dioxaborolan-2-yl)-1H-indazol-3-amine (0.046 g, 0.18 mmol), 1M potassium phosphate solution (0.24 ml, 0.24 mmol), [1,1′-bis(di-tert-butylphosphino)ferrocene]dichloropalladium(II) catalyst (0.0015 g, 0.006 mmol) in 0.7 ml ethanol were reacted as described in general procedure B and chromatographic purification (2% triethylamine in ethanol), gave the titled compound as yellow solid (19.1 mg, 45%). ¹H NMR (400 MHz, DMSO-d₆) δ ppm 2.08 (s, 6H) 2.31 (t, J=6.37 Hz, 2H) 2.54-2.62 (m, 2H), 3.86 (s, 2H) 5.51 (s, 2H) 6.55 (s, 1H) 7.13 (d, J=4.83 Hz, 1H) 7.37 (d, J=8.79 Hz, 1H) 7.64 (dd, J=8.57, 1.54 Hz, 1H) 8.14 (s, 1H) 8.17 (d, J=4.83 Hz, 1H) 11.60 (br. s, 1H). ¹³C NMR (100 MHz, DMSO-d₆) 45.72, 46.65, 46.82, 59.29, 97.78, 110.24, 114.37, 115.06, 118.23, 120.63, 127.11, 128.54, 138.78, 140.62, 141.58, 142.51, 149.97, 150.27. m/z (ESI-HRMS) calculated for C₁₉H₂₄N₇=350.2088 found=350.2086.

### Example 256

### N-((4-Chloro-7-azaindole)methyl)-N-(2-methoxypropyl)amine

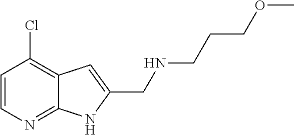 [SMILES: COCCCNCc1cc2c(Cl)ccnc2[nH]1]

4-Chloro-7-azaindole-2-carbaldehyde (100) (0.12 g, 0.66 mmol) was reacted with 2-methoxypropylamine (0.08 ml, 0.8 mmol), sodium triacetoxyborohydride (0.21 g, 0.99 mmol) and acetic acid (0.06 ml, 0.99 mmol) as described in general procedure A to afforded the desired compound as a brown oil (0.14 g, 83%). ¹H NMR (400 MHz, CDCl₃) δ 1.60-1.70 (m, 2H), 2.53-2.58 (m, 2H), 3.19 (s, 3H) 3.36 (br. s., 2H) 3.85 (s, 2H) 6.36 (s, 1H) 7.13 (d, J=4.83 Hz, 1H) 8.08 (d, J=4.83 Hz, 1H). ¹³C NMR (100 MHz, CDCl₃) δ ppm 29.91, 46.50, 46.08, 58.40, 69.68, 96.39, 115.71, 119.63, 133.37, 141.86, 142.86, 145.05. m/z (ESI-MS) [M]⁺ 254.1.

### 5-(2-(((3-methoxypropyl)amino)methyl)-1H-pyrrolo[2,3-b]pyridine-4-yl)-1H-indazol-3-amine

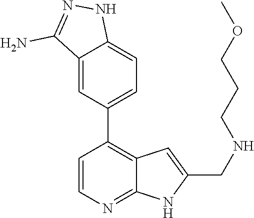 [SMILES: COCCCNCc1cc2c(-c3ccc4[nH]nc(N)c4c3)ccnc2[nH]1]

N-((4-chloro-7-azaindole)methyl)-N-(2-methoxypropyl)amine (0.09 g, 0.35 mmol), 5-(4,4,5,5-tetramethyl-1,3,2-dioxaborolan-2-yl)-1H-indazol-3-amine (0.138 g, 0.53 mmol), 1M potassium phosphate solution (0.71 ml, 0.71 mmol), [1,1′-bis(di-tert-butylphosphino)ferrocene]dichloropalladium(II) catalyst (0.004 g, 0.017 mmol) in 2.1 ml ethanol were reacted as described in general procedure B and chromatographic purification (10-15% methanol in EtOAc and 1% triethylamine), gave the titled compound as white solid (10.4 mg, 8%). ¹H NMR (400 MHz, DMSO-d₆) δ ppm 1.68 (quin, J=6.70 Hz 2H), 2.55-2.66 (m, 2H), 3.19 (s, 3H) 3.36 (m, 2H) 3.93 (m, 2H) 5.51 (s, 2H) 6.61 (s, 1H) 7.15 (d, J=5.27 Hz, 1H) 7.38 (d, J=8.79 Hz, 1H) 7.65 (dd, J=8.79, 1.76 Hz, 1H) 8.12-8.16 (m, 1H) 8.20 (d, J=4.83 Hz, 1H) 11.57 (s, 1H) 11.55 (s, 1H). ¹³C NMR (100 MHz, DMSO-d₆) 29.48, 46.01, 46.44, 58.40, 70.62, 96.44, 110.37, 114.58, 115.15, 118.28, 120.79, 127.19, 128.51, 140.74, 141.18, 141.66, 142.93, 149.97, 150.38. m/z (ESI-HRMS) calculated for C₁₉H₂₃ON₆=351.1928 found=351.1927.

### Example 257

### N-((4-Chloro-7-azaindole)methyl)-N-(3-isoprpoxypropyl)amine

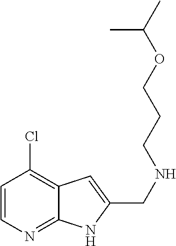 [SMILES: CC(C)OCCCNCc1cc2c(Cl)ccnc2[nH]1]

4-Chloro-7-azaindole-2-carbaldehyde (0.12 g, 0.66 mmol) was reacted with isoprpoxypropylamine (0.12 ml, 0.86 mmol), sodium triacetoxyborohydride (0.22 g, 0.99 mmol) and acetic acid (0.06 ml, 0.99 mmol) as described in general procedure A and chromatographic purification (30-100% EtOAc and 1% triethylamine in petroleum ether 60-80%) to afforded the desired compound as white solid (77.4 mg, 41%). ¹H NMR (400 MHz, DMSO-d₆) δ ppm 1.04 (d, J=6.02 Hz, 6H) 1.62 (quin, J=6.65 Hz, 2H) 2.56 (t, J=6.90 Hz, 2H) 3.40 (t, J=6.40 Hz, 2H) 3.48 (dt, J=12.11, 6.12 Hz, 1H) 3.84 (s, 2H) 6.36 (s, 1H) 7.13 (d, J=5.27 Hz, 1H) 8.09 (d, J=5.02 Hz, 1H) 11.85 (br. s., 1H). ¹³C NMR (100 MHz, DMSO-d₆) δ ppm 22.54, 30.56, 46.23, 46.57, 66.04, 70.94, 96.10, 115.61, 119.58, 133.25, 142.13, 142.70, 149.68. m/z (ESI-MS) [M]⁺ 282.1.

### 5-(2-(((3-Isopropoxypropyl)amino)methyl)-1H-pyrrolo[2,3-b]346yridine-4-yl)-1H-indazol-3-amine

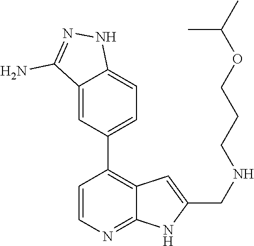 [SMILES: CC(C)OCCCNCc1cc2c(-c3ccc4[nH]nc(N)c4c3)ccnc2[nH]1]

N-((4-Chloro-7-azaindole)methyl)-N-(3-isoprpoxypropyl)amine (0.05 g, 0.13 mmol), 5-(4,4,5,5-tetramethyl-1,3,2-dioxaborolan-2-yl)-1H-indazol-3-amine (0.07 g, 0.27 mmol), 1M potassium phosphate solution (0.64 ml, 0.64 mmol), [1,1′-bis(di-tert-butylphosphino)ferrocene]dichloropalladium(II) catalyst (0.002 g, 0.006 mmol) in 1.9 ml ethanol were reacted as described in general procedure B and chromatographic purification (0-10% methanol in EtOAc and 1% triethylamine), gave the titled compound as yellow solid (10.6 mg, 21%), ¹H NMR (400 MHz, DMSO-d₆) δ ppm 1.02 (d, J=6.27 Hz, 6H) 1.62 (quin, J=6.59 Hz, 2H) 2.54-2.61 (m, 2H), 3.39 (t, J=6.53 Hz, 2H) 3.42-3.51 (m, 1H) 3.86 (s, 2H) 5.49 (s, 2H) 6.56 (s, 1H) 7.13 (d, J=5.02 Hz, 1H) 7.38 (d, J=8.78 Hz, 1H) 7.66 (dd, J=8.66, 1.63 Hz, 1H) 8.14 (s, 1H) 8.18 (d, J=5.02 Hz, 1H) 11.48-1154 (m, 2H). ¹³C NMR (100 MHz, DMSO-d₆) 22.52, 30.51, 46.25, 46.77, 66.07, 70.93, 96.49, 110.22, 114.39, 115.42, 119.29, 120.63, 127.09, 128.65, 138.77, 139.80, 140.09, 140.19, 149.92, 151.80. m/z (ESI-HRMS) calculated for C₂₁H₂₇ON₆=379.2241 found=379.2243.

### Example 258

### N¹-((4-chloro-7-azaindole)methyl)-N²,N²-dimethyl-1,2-propanediamine

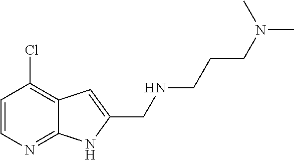 [SMILES: CN(C)CCCNCc1cc2c(Cl)ccnc2[nH]1]

4-Chloro-7-azaindole-2-carbaldehyde (0.12 g, 0.66 mmol) was reacted N¹,N¹dimethyl-1,2propanediamine (0.1 ml, 0.08 mmol), sodium triacetoxyborohydride (0.21 g, 0.99 mmol) and acetic acid (0.06 ml, 0.99 mmol) as described in general procedure A to afforded the desired compound as a whit solid (0.08 g, 45%), ¹H NMR (400 MHz, CDCl₃) δ 1.47-1.60 (m, 2H), 1.85 (s, 6H), 2.20-2.25 (m, 2H) 3.17 (s, 2H) 3.83 (s, 2H) 6.35 (s, 1H) 7.13 (d, J=5.27 Hz, 1H) 8.08 (d, J=5.27 Hz, 1H) 11.49 (s, 1H). m/z (ESI-MS) [M]⁺ 267.1.

### N¹-((4-(3-amino-1H-indazol-5-yl)-1H-pyrrolo[2,3-b]pyridin-2-yl)methyl)-N³,N³-dimethylpropane-1,3-diamine

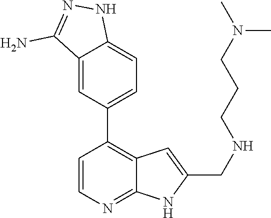 [SMILES: CN(C)CCCNCc1cc2c(-c3ccc4[nH]nc(N)c4c3)ccnc2[nH]1]

N¹-((4-Chloro-7-azaindole)methyl)-N²,N²-dimethyl-1,2-propanediamine (0.05 g, 0.18 mmol), 5-(4,4,5,5-tetramethyl-1,3,2-dioxaborolan-2-yl)-1H-indazol-3-amine (0.073 g, 0.28 mmol), 1M potassium phosphate solution (0.36 ml, 0.36 mmol), [1,1′-bis(di-tert-butylphosphino)ferrocene]dichloropalladium(II) catalyst (0.002 g, 0.009 mmol) in 1.08 ml ethanol were reacted as described in general procedure B and chromatographic purification (100% ethanol and 1% triethylamine, further purification by HPLC gave the titled compound as yellow solid (27.5 mg, 32%). ¹H NMR (400 MHz, DMSO-d₆) δ ppm 1.96-2.07 (m, 2H), 2.77 (s, 6H), 3.04 (br. s, 2H) 3.13 (t, J=7.53 Hz, 2H) 4.40 (br. s., 2H) 6.91 (s, 1H) 7.26 (d, J=5.02 Hz, 1H) 7.43 (d, J=8.53 Hz, 1H) 7.71 (dd, J=8.66, 1.63 Hz, 1H) 8.19 (s, 1H) 8.34 (d, J=5.02 Hz, 1H) 11.88 (br. s., 2H). m/z (ESI-HRMS) calculated for C₂₀H₂₆N₇=364.2244 found=364.2243.

### Example 259

### N-((4-Chloro-7-azaindole)methyl)-N-isopropyl-N-methylamine

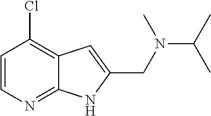 [SMILES: CC(C)N(C)Cc1cc2c(Cl)ccnc2[nH]1]

4-Chloro-7-azaindole-2-carbaldehyde (0.1 g, 0.55 mmol) was reacted with isopropylmethylamine (0.07 ml, 0.72 mmol), sodium triacetoxyborohydride (0.17 g, 0.82 mmol) and acetic acid (0.05 ml, 0.83 mmol) as described in general procedure A to afford the desired compound as a yellow solid (110 g, 84%), ¹H NMR (400 MHz, DMSO-d₆) δ ppm 1.02 (d, J=6.59 Hz, 6H) 2.13 (s, 3H) 2.81-2.90 (m, 2H) 3.65 (s, 2H) 6.34 (s, 1H) 7.13 (d, J=5.27 Hz, 1H) 8.09 (d, J=5.27 Hz, 1H) 11.88 (br. s., 1H). ¹³C NMR (100 MHz, DMSO-d₆) δ ppm 18.19, 36.96, 51.09, 52.93, 96.95, 115.71, 119.32, 131.29, 141.14, 142.89, 149.82. m/z (ESI-MS) [M−dialkylamine]⁺ 165.1 (100%) [M]⁺ 238.1 (20%).

### 5-(2-((Isopropyl(methyl)amino)methyl)-1H-pyrrolo[2,3-b]pyridine-4-yl)-1H-indazol-3-amine

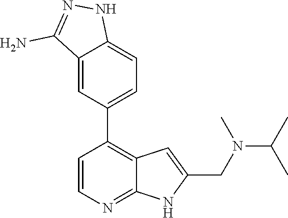 [SMILES: CC(C)N(C)Cc1cc2c(-c3ccc4[nH]nc(N)c4c3)ccnc2[nH]1]

N-((4-Chloro-7-azaindole)methyl)-N-isopropyl-N-methylamine (0.05 g, 0.21 mmol), 5-(4,4,5,5-tetramethyl-1,3,2-dioxaborolan-2-yl)-1H-indazol-3-amine (0.08 g, 0.315 mmol), 1M potassium phosphate solution (0.42 ml, 0.42 mmol), [1,1′-bis(di-tert-butylphosphino)ferrocene]dichloropalladium(II) catalyst (0.006 g, 0.01 mmol) in 1.2 ml ethanol were reacted as described in general procedure B and chromatographic purification (0-18% methanol in EtOAc and 1% triethylamine), gave the titled compound as brown solid (46.1 mg, 65%). ¹H NMR (400 MHz, DMSO-d₆) δ ppm 1.01 (d, J=6.59 Hz, 6H), 2.13 (s, 3H) 2.43 (q, J=7.03 Hz, 1H) 3.67 (s, 2H) 5.52 (br. s, 2H) 6.54 (s, 1H) 7.13 (d, J=4.83 Hz, 1H) 7.38 (d, J=8.79 Hz, 1H) 7.64 (dd, J=8.35, 1.32 Hz, 1H) 8.13 (s, 1H) 8.18 (d, J=4.83 Hz, 1H) 11.56 (s, 1H). ¹³C NMR (100 MHz, DMSO-d₆) 18.07, 36.73, 51.32, 52.72, 98.49, 110.29, 114.37, 115.04, 118.25, 120.63, 127.14, 128.54, 139.55, 140.81, 141.57, 142.53, 149.93, 150.27. m/z (ESI-HRMS) calculated for C₁₉H₂₃N₆=335.1979 found=335.1981.

### Example 260

### N-((4-Chloro-7-azaindole)methyl)-piperdine

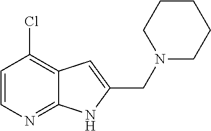 [SMILES: Clc1ccnc2[nH]c(CN3CCCCC3)cc12]

4-Chloro-7-azaindole-2-carbaldehyde (0.12 g, 0.66 mmol) was reacted with piperidine (0.08 ml, 0.86 mmol), sodium triacetoxyborohydride (0.22 g, 0.99 mmol) and acetic acid (0.06 ml, 0.99 mmol) as described in general procedure A and chromatographic purification (30-100% EtOAc and 1% triethylamine in petroleum ether 60-80%) to afforded the desired compound as white solid (77.2 mg, 47%), ¹H NMR (400 MHz, DMSO-d₆) δ ppm 1.37 (m, 2H) 1.50 (q, J=5.49 Hz, 4H) 2.38 (br. s., 4H) 3.59 (s, 2H) 6.33 (d, J=1.76 Hz, 1H) 7.14 (d, J=4.83 Hz, 1H) 8.10 (d, J=5.27 Hz, 1H) 11.96 (br. s., 1H). ¹³C NMR (100 MHz, DMSO-d₆) δ ppm 24.43, 26.06, 54.40, 56.11, 97.58, 115.76, 119.53, 133.47, 139.32, 143.03, 149.74. m/z (ESI-MS) [M]⁺ 250.2.

### 5-(2-(Piperidin-1-ylmethyl)-1H-pyrrolo[2,3-b]pyridine-4-yl)-1H-indazol-3-amine

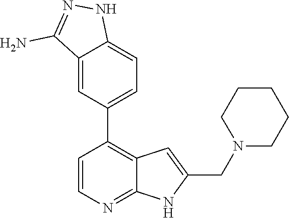 [SMILES: Nc1n[nH]c2ccc(-c3ccnc4[nH]c(CN5CCCCC5)cc34)cc12]

N-((4-Chloro-7-azaindole)methyl)-piperdine (0.043 g, 0.17 mmol), 5-(4,4,5,5-tetramethyl-1,3,2-dioxaborolan-2-yl)-1H-indazol-3-amine (0.067 g, 0.26 mmol), 1M potassium phosphate solution (0.34 ml, 0.34 mmol), [1,1′-bis(di-tert-butylphosphino)ferrocene]dichloropalladium(II) catalyst (0.002 g, 0.008 mmol) in 1.0 ml ethanol were reacted as described in general procedure B and chromatographic purification (0-10% methanol in EtOAc and 1% triethylamine), gave the titled compound as white solid (30.0 mg, 51%), ¹H NMR (400 MHz, DMSO-d₆) δ ppm 1.31-1.41 (m, 2H), 1.44-1.53 (m, 4H), 2.38 (br. s., 4H) 3.60 (s, 2H) 5.53 (s, 2H) 6.53 (d, J=1.76 Hz, 1H) 7.13 (d, J=4.83 Hz, 1H) 7.38 (d, J=8.79 Hz, 1H) 7.64 (dd, J=8.57, 1.54 Hz, 1H) 8.14 (s, 1H) 8.19 (d, J=4.83 Hz, 1H) 11.55 (br. s, 1H) 11.67 (s, 1H). ¹³C NMR (100 MHz, DMSO-d₆) 24.48, 26.06, 54.44, 56.45, 99.28, 110.39, 114.49, 115.14, 118.25, 120.77, 127.24, 128.57, 137.88, 141.03, 141.65, 142.78, 149.95, 150.39. m/z (ESI-HRMS) calculated for C₂₀H₂₃N₆=347.1979 found=347.1976.

### Example 261

### N-((4-Chloro-7-azaindole)methyl)-4,4-difluoropiperidine

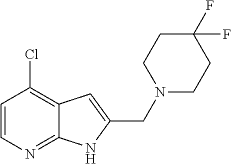 [SMILES: FC1(F)CCN(Cc2cc3c(Cl)ccnc3[nH]2)CC1]

4-Chloro-7-azaindole-2-carbaldehyde (0.12 g, 0.66 mmol) was reacted with 4,4-difluoropiperidine hydrochloride (0.12 gm, 1.0 mmol), sodium triacetoxyborohydride (0.21 g, 0.99 mmol) and triethylamine (0.14 ml, 1 mmol) as described in general A procedure to afforded the desired compound as a white solid (0.043 g, 22%). ¹H NMR (400 MHz, CDCl₃) δ 1.92-2.03 (m, 4H), 2.55 (t, J=5.27 Hz, 4H), 3.74 (s, 2H) 6.39 (d, J=1.76 Hz, 1H) 7.16 (d, J=5.27 Hz, 1H) 8.12 (d, J=5.27 Hz, 1H) 12.00 (br. s., 1H). ¹³C NMR (100 MHz, CDCl₃) δ ppm 33.97, 49.83, 54.21, 97.74, 100.44, 115.86, 119.54, 133.65, 138.77, 143.24, 149.75. ¹⁹F NMR (DMSO-d₆) −95.77. m/z (ESI-MS) [M]⁺ 286.1.

### 5-(2-((4,4-Difluoropiperidin-1-yl)methyl)-1H-pyrrolo[2,3-b]pyridine-4-yl)-1H-indazol-3-amine

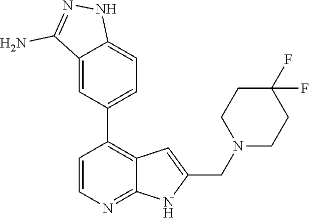 [SMILES: Nc1n[nH]c2ccc(-c3ccnc4[nH]c(CN5CCC(F)(F)CC5)cc34)cc12]

N-((4-Chloro-7-azaindole)methyl)-4,4-difluoropiperidine (0.027 g, 0.09 mmol), 5-(4,4,5,5-tetramethyl-1,3,2-dioxaborolan-2-yl)-1H-indazol-3-amine (0.036 g, 0.14 mmol), 1M potassium phosphate solution (0.18 ml, 0.18 mmol), [1,1′-bis(di-tert-butylphosphino)ferrocene]dichloropalladium(II) catalyst (0.001 g, 0.0045 mmol) in 0.54 ml ethanol were reacted as described in general procedure B and chromatographic purification (5-10% methanol in EtOAc and 1% triethylamine), gave the titled compound as reddish solid (14.3 mg, 41%), ¹H NMR (400 MHz, DMSO-d₆) δ ppm 1.92-2.03 (m, 4H), 2.57 (br. s., 4H), 3.75 (s, 2H) 5.51 (s, 2H) 6.59 (s, 1H) 7.15 (d, J=5.06 Hz, 1H) 7.39 (d, J=8.36 Hz, 1H) 7.66 (d, J=6.82 Hz, 1H) 8.15 (s, 1H) 8.21 (d, J=5.06 Hz, 1H). ¹³C NMR (100 MHz, DMSO-d₆) 33.41, 49.26, 53.95, 98.85, 101.35, 109.80, 113.99, 114.57, 117.67, 120.18, 126.63, 127.94, 136.73, 140.63, 141.09, 142.39, 149.39, 149.57. ¹⁹F NMR (DMSO-d₆) −95.77. m/z (ESI-HRMS) calculated for C₂₀H₂₁N₆F₂=383.1790 found=383.1795.

### Example 262

### N-((4-Chloro-7-azaindole)methyl)-morpholine

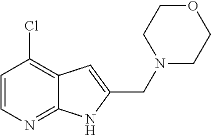 [SMILES: Clc1ccnc2[nH]c(CN3CCOCC3)cc12]

4-Chloro-7-azaindole-2-carbaldehyde (0.12 g, 0.66 mmol) was reacted with morpholine (0.07 ml, 0.86 mmol), sodium triacetoxyborohydride (0.21 g, 0.99 mmol) and acetic acid (0.06 ml, 0.99 mmol) as described in general procedure A to afford the desired compound as a white solid (116 mg, 70%). ¹H NMR (400 MHz, DMSO-d₆) δ ppm 2.38-2.44 (m, 4H) 3.56-4.61 (m, 4H) 3.64 (s, 2H) 6.37 (s, 1H) 7.15 (d, J=5.27 Hz, 1H) 8.11 (d, J=5.27 Hz, 1H) 12.03 (br. s., 1H). ¹³C NMR (100 MHz, DMSO-d₆) δ ppm 53.65, 55.64, 66.70, 97.84, 115.80, 119.50, 133.58, 138.58, 143.19, 149.78. m/z (ESI-MS) [M]⁻ 250.1.

### 5-(2-(Morpholinomethyl)-1H-pyrrolo[2,3-b]pyridine-4-yl)-1H-indazol-3-amine

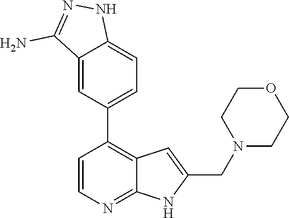 [SMILES: Nc1n[nH]c2ccc(-c3ccnc4[nH]c(CN5CCOCC5)cc34)cc12]

N-((4-Chloro-7-azaindole)methyl)-morpholine (0.055 g, 0.22 mmol), 5-(4,4,5,5-tetramethyl-1,3,2-dioxaborolan-2-yl)-1H-indazol-3-amine (0.085 g, 0.33 mmol), 1M potassium phosphate solution (0.43 ml, 0.43 mmol), [1,1′-bis(di-tert-butylphosphino)ferrocene]dichloropalladium(II) catalyst (0.007 g, 0.01 mmol) in 1.3 ml ethanol were reacted as described in general procedure B and chromatographic purification (0-15% methanol in EtOAc and 1% triethylamine), gave the titled compound as a reddish solid (42.8 mg, 56%)¹H NMR (400 MHz, DMSO-d₆) δ ppm 2.43 (br. s., 4H), 3.58 (t, J=4.39 Hz, 4H) 3.65 (s, 2H) 5.53 (s, 2H) 6.57 (d, J=1.76 Hz, 1H) 7.14 (d, J=4.83 Hz, 1H) 7.38 (d, J=8.79 Hz, 1H) 7.65 (dd, J=8.57, 1.54 Hz, 1H) 8.14 (s, 1H) 8.20 (d, J=4.83 Hz, 1H) 11.54 (br. s, 1H) 11.67 (s, 1H). ¹³C NMR (100 MHz, DMSO-d₆) 53.62, 55.92, 66.61, 99.48, 110.30, 114.45, 115.06, 118.14, 120.68, 127.14, 128.46, 136.99, 141.07, 142.68, 142.84, 149.85, 150.29. m/z (ESI-HRMS) calculated for C₁₉H₂₁ON₆=349.1771 found=349.1774.

### Example 263

### 1-((4-Chloro-7-azaindole)methyl)-4-methylpiperazine

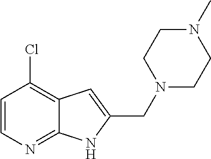 [SMILES: CN1CCN(Cc2cc3c(Cl)ccnc3[nH]2)CC1]

4-Chloro-7-azaindole-2-carbaldehyde (0.12 g, 0.66 mmol) was reacted with 4-methylpiperazine (0.09 ml, 0.86 mmol), sodium triacetoxyborohydride (0.22 g, 0.99 mmol) and acetic acid (0.06 ml, 0.99 mmol) as described in general procedure A and chromatographic purification (30-100% EtOAc and 1% triethylamine in petroleum ether 60-80%) to afforded the desired compound as white solid (107.13 mg, 61%). ¹H NMR (400 MHz, DMSO-d₆) δ ppm 1.77 (s, 8H) 2.15 (s, 3H) 3.62 (s, 2H) 6.35 (s, 1H) 7.14 (d, J=5.27 Hz, 1H) 8.10 (d, J=4.83 Hz, 1H) 11.89 (br. s., 1H). ¹³C NMR (100 MHz, DMSO-d₆) δ ppm 21.72, 46.21, 55.18, 97.73, 115.79, 119.51, 132.57, 138.39, 143.13, 150.75 m/z (ESI-MS) [M]⁺ 265.1.

### 5-(2-((4-Methylpiperazin-1-yl)methyl)-1H-pyrrolo[2,3-b]pyridine-4-yl)-1H-indazol-3-amine

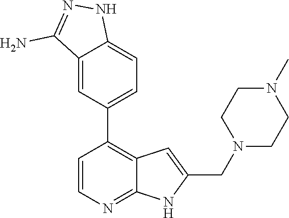 [SMILES: CN1CCN(Cc2cc3c(-c4ccc5[nH]nc(N)c5c4)ccnc3[nH]2)CC1]

1-((4-Chloro-7-azaindole)methyl)-4-methylpiperazine (0.05 g, 0.19 mmol), 5-(4,4,5,5-tetramethyl-1,3,2-dioxaborolan-2-yl)-1H-indazol-3-amine (0.07 g, 0.28 mmol), 1M potassium phosphate solution (0.4 ml, 0.4 mmol), [1,1′-bis(di-tert-butylphosphino)ferrocene]dichloropalladium(II) catalyst (0.002 g, 0.01 mmol) in 1.2 ml ethanol were reacted as described in general procedure B and chromatographic purification (2% triethylamine in ethanol), gave the titled compound as white solid (40.4 mg, 61%), ¹H NMR (400 MHz, DMSO-d₆) δ ppm 1.74 (s, 8H) 2.14 (s, 3H) 3.64 (s, 2H) 5.50 (s, 2H) 6.56 (s, 1H) 7.14 (d, J=5.02 Hz, 1H) 7.39 (d, J=8.53 Hz, 1H) 7.65 (dd, J=8.53, 1.51 Hz, 1H) 8.13-8.16 (m, 1H) 8.20 (d, J=5.02 Hz, 1H) 11.66 (br. s, 2H). ¹³C NMR (100 MHz, DMSO-d₆) 46.20, 53.05, 55.12, 55.58, 99.31, 110.33, 114.41, 115.03, 118.15, 120.66, 127.10, 128.45, 137.49, 141.03, 141.61, 142.75, 149.90, 150.25 m/z (ESI-HRMS) calculated for C₂₀H₂₄N₇=362.2088 found=362.2085.

### Example 264

### 1-((4-Chloro-7-azaindole)methyl)-4-(tert-butyl)piperazine

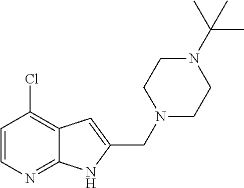 [SMILES: CC(C)(C)N1CCN(Cc2cc3c(Cl)ccnc3[nH]2)CC1]

4-Chloro-7-azaindole-2-carbaldehyde (0.12 g, 0.66 mmol) was reacted with N-(tert-butyl)piperazine (0.122 ml, 0.86 mmol), sodium triacetoxyborohydride (0.21 g, 0.99 mmol) and acetic acid (0.06 ml, 0.99 mmol) as described in general procedure A to afforded the desired compound as a white solid (130 mg, 76%). ¹H NMR (400 MHz, DMSO-d₆) δ ppm 0.98 (s, 9H) 2.42 (br. s., 4H) 3.61 (s, 2H) 6.33 (s, 1H) 7.14 (d, J=5.27 Hz, 1H) 8.10 (d, J=4.83 Hz, 1H). ¹³C NMR (100 MHz, DMSO-d₆) δ ppm 25.65, 45.07, 52.92, 53.41, 54.71, 97.03, 115.17, 118.94, 132.92, 138.46, 142.48, 149.14. m/z (ESI-MS) [M]⁺ 307.1.

### 5-(2-((4-(tert-Butyl)piperazin-1-yl)methyl)-1H-pyrrolo[2,3-b]pyridin-4-yl)-1H-indazol-3-amine

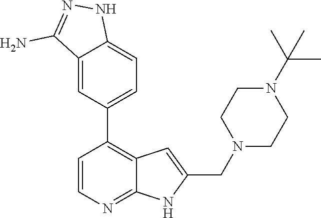 [SMILES: CC(C)(C)N1CCN(Cc2cc3c(-c4ccc5[nH]nc(N)c5c4)ccnc3[nH]2)CC1]

1-((4-Chloro-7-azaindole)methyl)-4-(tert-butyl)piperazine (0.05 g, 0.16 mmol), 5-(4,4,5,5-tetramethyl-1,3,2-dioxaborolan-2-yl)-1H-indazol-3-amine (0.063 g, 0.24 mmol), 1M potassium phosphate solution (0.32 ml, 0.32 mmol), [1,1′-bis(di-tert-butylphosphino)ferrocene]dichloropalladium(II) catalyst (0.006 g, 0.008 mmol) in 1 ml ethanol were reacted as described in general procedure and chromatographic purification (1% triethylamine in EtOAc), gave the titled compound as white solid (56 mg, 86%), ¹H NMR (400 MHz, DMSO-d₆) δ ppm 0.97 (s, 9H) 2.43 (br. s., 4H) 3.62 (s, 2H) 5.53 (s, 2H) 6.54 (s, 1H) 7.13 (d, J=4.83 Hz, 1H) 7.38 (d, J=8.79 Hz, 1H) 7.65 (d, J=10.11, 1H) 8.14 (s, 1H) 8.19 (d, J=4.83 Hz, 1H) 11.55 (s, 1H). ¹³C NMR (100 MHz, DMSO-d₆) δ ppm 25.64, 45.07, 52.94, 53.45, 55.05, 98.73, 109.79, 113.92, 114.56, 117.66, 120.16, 126.64, 127.99, 136.99, 140.49, 141.08, 142.23, 149.37, 149.78. m/z (ESI-HRMS) calculated for C₂₃H₃₀N₇=404.2557 found=404.2562.

### Example 265

### N-((4-Chloro-7-azaindole)methyl)-azapane

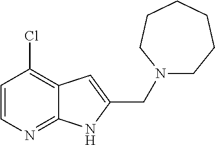 [SMILES: Clc1ccnc2[nH]c(CN3CCCCCC3)cc12]

4-Chloro-7-azaindole-2-carbaldehyde (0.12 g, 0.66 mmol) was reacted with azapane (0.07 ml, 0.86 mmol), sodium triacetoxyborohydride (0.21 g, 0.99 mmol) and acetic acid (0.06 ml, 0.99 mmol) as described in general procedure A to afforded the desired compound as a white solid (0.09 g, 51%), ¹H NMR (400 MHz, CDCl₃) δ 1.57 (br. s., 8H), 2.57-2.66 (m, 4H), 3.76 (s, 2H) 6.31 (s, 1H) 7.14 (d, J=4.03 Hz, 1H) 8.09 (d, J=5.49 Hz, 1H) 11.93 (br. s., 1H). ¹³C NMR (100 MHz, CDCl₃) δ ppm 27.02, 28.39, 55.41, 55.48, 97.07, 115.73, 119.60, 133.42, 140.63, 142.93, 149.75. m/z (ESI-MS) [M]⁺ 264.1.

### 5-(2-(Azepan-1-ylmethyl)-1H-pyrrolo[2,3-b]pyridine-4-yl)-1H-indazol-3-amine

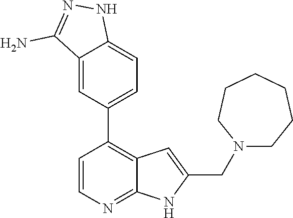 [SMILES: Nc1n[nH]c2ccc(-c3ccnc4[nH]c(CN5CCCCCC5)cc34)cc12]

N-((4-Chloro-7-azaindole)methyl)-azapane (0.066 g, 0.25 mmol), 5-(4,4,5,5-tetramethyl-1,3,2-dioxaborolan-2-yl)-1H-indazol-3-amine (0.097 g, 0.37 mmol), 1M potassium phosphate solution (0.5 ml, 0.5 mmol), [1,1′-bis(di-tert-butylphosphino)ferrocene]dichloropalladium(II) catalyst (0.003 g, 0.012 mmol) in 1.5 ml ethanol were reacted as described in general procedure B and chromatographic purification (0-5% methanol in EtOAc and 1% triethylamine), gave the titled compound as white solid (39.1 mg, 43%), ¹H NMR (400 MHz, DMSO-d₆) δ ppm 1.56 (br. s., 8H), 2.57-2.66 (m, 4H), 3.77 (s, 2H) 5.52 (s, 2H) 6.54 (s, 1H) 7.13 (d, J=5.27 Hz, 1H) 7.38 (d, J=8.35 Hz, 1H) 7.65 (dd, J=8.57, 1.10 Hz, 1H) 8.14 (s, 1H) 8.18 (d, J=4.83 Hz, 1H) 11.54 (s, 1H) 11.58 (s, 1H). ¹³C NMR (100 MHz, DMSO-d₆) 27.04, 28.34, 55.46, 55.69, 98.80, 110.38, 114,49, 115.14, 118.31. 120.76, 127.23, 128.61, 139.13, 140.98, 141.65, 142.69, 149.98, 150.39. m/z (ESI-HRMS) calculated for C₂₁H₂₅N₆=361.2135 found=361.2135.

### Example 266

### 1-((4-Chloro-7-azaindole)methyl)-4-methyl-1,4-diazapane

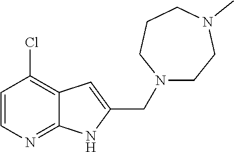 [SMILES: CN1CCCN(Cc2cc3c(Cl)ccnc3[nH]2)CC1]

4-Chloro-7-azaindole-2-carbaldehyde (0.12 g, 0.66 mmol) was reacted with 4-methylazapane (0.09 ml, 0.86 mmol), sodium triacetoxyborohydride (0.21 g, 0.99 mmol) and acetic acid (0.06 ml, 0.99 mmol) as described in general procedure A to afforded the desired compound as a white solid (0.05 g, 27%), ¹H NMR (400 MHz, CDCl₃) δ 1.65-1.74 (m, 2H), 1.85 (s, 4H), 2.23 (s, 3H) 2.61-2.74 (m, 4H) 3.77 (s, 2H) 6.35 (s, 1H) 7.15 (d, J=4.03 Hz, 1H) 8.08 (d, J=5.49 Hz, 1H) 11.92 (br. s., 1H) 11.97 (s, 1H). ¹³C NMR (100 MHz, CDCl₃) δ ppm 27.48, 47.13, 54.27, 54.72, 55.28, 56.72, 57.70, 97.22, 115.76, 119.58, 133.46, 140.31, 142.99, 149.91. m/z (ESI-MS) [M]⁺ 279.1.

### 5-(2-((4-Methyl-1,4-diazepan-1-yl)methyl)-1H-pyrrolo[2,3-b]pyridin-4-yl)-1H-indazol-3-amine

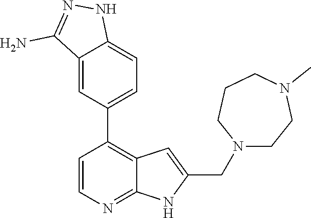 [SMILES: CN1CCCN(Cc2cc3c(-c4ccc5[nH]nc(N)c5c4)ccnc3[nH]2)CC1]

1-((4-Chloro-7-azaindole)methyl)-4-methyl-1,4diazapane (0.038 g, 0.13 mmol), 5-(4,4,5,5-tetramethyl-1,3,2-dioxaborolan-2-yl)-1H-indazol-3-amine (0.053 g, 0.2 mmol), 1M potassium phosphate solution (0.26 ml, 0.26 mmol), [1,1′-bis(di-tert-butylphosphino)ferrocene]dichloropalladium(II) catalyst (0.0016 g, 0.065 mmol) in 0.78 ml ethanol were reacted as described in general procedure B and chromatographic purification (18% methanol in EtOAc and 1% triethylamine), gave the titled compound as white solid (48 mg, 98%), ¹H NMR (400 MHz, DMSO-d₆) δ ppm 1.65-1.74 (m, 2H), 1.85 (s, 4H), 2.23 (s, 3H) 2.61-2.74 (m, 4H) 3.77 (s, 2H) 5.52 (br. s., 2H) 6.55 (s, 1H) 7.13 (d, J=5.27 Hz, 1H) 7.38 (d, J=8.79 Hz, 1H) 7.65 (dd, J=8.57, 1.54 Hz, 1H) 8.14 (s, 1H) 8.19 (d, J=5.27 Hz, 1H) 11.61 (br. s., 1H). ¹³C NMR (100 MHz, DMSO-d₆) 27.42, 47.10, 54.28, 54.63 55.55, 56.74, 58.21, 98.98, 110.39, 114.49, 115.14, 118.29, 120.76, 127,23, 128.58, 138.81, 141.01, 141.66, 142.75, 150.01, 150.37. m/z (ESI-HRMS) calculated for C₂₁H₂₆N₇=376.2244 found=376.2245.

### Example 267

### 4-Chloro-3-(4-(piperidin-1-yl)but-1-yn-1yl)pyridin-2-amine

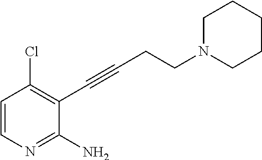 [SMILES: Nc1nccc(Cl)c1C#CCCN1CCCCC1]

A solution of 4-chloro-3-iodo-pyridine-2-amine (0.15 g, 0.6 mmol), copper (1) iodide (0.005 g, 0.03 mmol) and bis(triphenylphosphine) palladium(II) chloride (0.02 g, 0.03 mmol) in 4 ml of N,N-dimethyformamide-triethylamine (1:4) was degassed in a sealed tube followed by addition of 4-(but-3-yn-1-yl)piperidine (0.12 ml, 0.88 mmol). The reaction mixture was stirred at 80° C. for 4 h. The reaction mixture was diluted with EtOAc and extracted with 1M sodium carbonate. The extracted organic layer was washed with brine and dried over magnesium sulfate and concentrated under reduced pressure and the residue purified by column chromatography (80% EtOAc and 1% triethylamine in petroleum ether 60-80%) to give the product as yellow oil (0.063 g, 40%), 1H NMR (400 MHz, DMSO-d₆) δ ppm 1.39 (d, J=5.19 Hz, 2H) 1.52 (quin, J=5.57 Hz, 4H) 2.39 (br. s., 4H) 2.52-2.55 (m, 2H) 2.64-2.72 (m, 2H) 6.65 (d, J=5.34 Hz, 3H) 6.83 (d, J=5.49 Hz, 1H). ¹³C NMR (100 MHz, DMSO-d₆) δ ppm 18.07, 24.52, 25.83, 54.08, 57.34, 74.94, 101.20, 102.19, 112.47, 143.22, 148.05, 161.96. m/z (ESI-MS) [M]⁺ 264.0.

### 4-(2-(4-Chloro-7-azaindole-2-yl)ethyl)piperidine

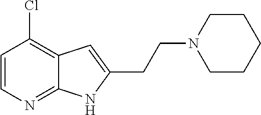 [SMILES: Clc1ccnc2[nH]c(CCN3CCCCC3)cc12]

To a solution of 4-chloro-3-(4-piperidin-1-yl)but-1-yn-1yl)pyridin-2-amine (128) (0.057 g, 0.19 mmol) in 1,4 dioxane (1 ml), potassium tert-butoxide (0.05 g, 0.4 mmol) and 18-crown-6 (0.005 g, 10%, 0.02 mmol) were added and the mixture was stirred at 120° C. for 18 h. After the reaction was cooled to room temperature, EtOAc and 1M sodium carbonate solution were added. Extracted organic layer was dried over magnesium sulfate and concentrated under reduced pressure to give the product as yellow solid (52.6 mg, 93%), 1H NMR (400 MHz, DMSO-d₆) δ ppm 1.31-1.45 (m, 2H) 1.45-154 (m, 4H) 2.40 (br. s., 4H) 2.63 (t, J=7.69 Hz, 2H) 2.89 (t, J=7.69 Hz, 2H) 6.26 (s, 1H) 7.11 (d, J=5.27 Hz, 1H) 8.06 (d, J=5.27 Hz, 1H) 11.90 (br. s., 1H). ¹³C NMR (100 MHz, DMSO-d₆) δ ppm 24.66, 26.17, 54.37, 58.26, 70.06, 95.83, 115.66, 119.92, 132.95, 141.84, 142.43, 149.70. m/z (ESI-MS) [M]⁺ 264.1.

### 5-(2-(2-(Piperidin-1-yl)ethyl)-1H-pyrrolo[2,3-b]pyridine-4-yl)-1H-indazol-3-amine

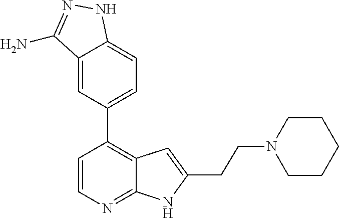 [SMILES: Nc1n[nH]c2ccc(-c3ccnc4[nH]c(CCN5CCCCC5)cc34)cc12]

4-(2-(4-Chloro-7-azaindole-2-ethyl)piperidine (0.045 g, 0.17 mmol) 5-4,4,5,5-tetramethyl-1,3,2-dioxaborolan-2-yl)-1H-indazol-3-amine (0.06 g, 0.25 mmol), 1M potassium phosphate solution (0.3 ml, 0.3 mmol), [1,1′-bis(di-tert-butylphosphino)ferrocene]dichloropalladium(II) catalyst (0.002 g, 0.008 mmol) in 1.0 ml ethanol were reacted as described in general procedure B and chromatographic purification (90% EtOAc and 1% triethylamine in petroleum ether 60-80%), gave the titled compound as white solid (38 mg, 62%), ¹H NMR (400 MHz, DMSO-d₆) δ ppm 1.32-1.45 (m, 2H) 1.45-1.60 (m, 4H) 2.43 (br. s., 4H) 2.68 (br. s., 2H) 2.92 (t, J=6.59 Hz, 2H) 5.51 (s, 2H) 6.47 (s, 1H) 7.12 (d, J=4.83 Hz, 1H) 7.37 (d, J=8.79 Hz, 1H) 7.64 (dd, J=8.79, 1.76 Hz, 1H) 8.02-8.26 (m, 2H) 11.51-11.66 (m, 2H). ¹³C NMR (100 MHz, DMSO-d₆) δ ppm 25.95, 53.71, 58.17, 66.76, 97.50, 110.34, 114.46, 115.14, 118.61, 120.70, 127.20, 128.66, 139.99, 140.55, 141.63, 142.28, 149.96, 150.38. m/z (ESI-HRMS) calculated for C₂₁H₂₅N=361.2135 found=361.2132.

### Example 268

### 4-Chloro-3-(5-morpholinopent-1-yn-1yl)pyridin-2-amine

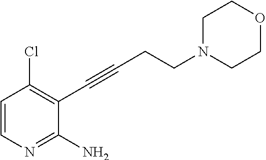 [SMILES: Nc1nccc(Cl)c1C#CCCN1CCOCC1]

A solution of 4-chloro-3-iodo-pyridine-2-amine (0.15 g, 0.6 mmol), copper (I) iodide (0.005 g, 0.03 mmol) and bis(triphenylphosphine) palladium(II) chloride (0.02 g, 0.03 mmol) in 4 ml of N,N-dimethyformamide-triethylamine (1:4) was degassed in a sealed tube followed by addition of 4-(but-3-yn-1-yl)morpholine (0.12 ml, 0.88 mmol) and stirred at 80° C. for 3 h. The reaction mixture was diluted with EtOAc and extract with 1 M sodium carbonate. The extracted organic layer was washed with brine and dried over magnesium sulfate and concentrated under reduced pressure and the residue purified by column chromatography (80% EtOAc and 1% triethylamine in petroleum ether 60-80%) to give the product as yellow oil (0.139 g, 89%), 1H NMR (400 MHz, DMSO-d₆) δ ppm 2.43 (br. s., 4H) 2.56 (t, J=6.81 Hz, 2H) 3.70 (t, J=6.81 Hz, 2H) 3.59 (t, J=4.61 Hz, 4H) 6.51-6.78 (m, 3H) 6.84 (d, J=5.27 Hz, 1H). ¹³C NMR (100 MHz, DMSO-d₆) δ ppm 17.85, 53.45, 57.05, 66.58, 75.05, 101.03, 102.25, 112.61, 143.38, 148.20, 162.01. m/z (ESI-MS) [M]⁺ 266.0.

### 4-(2-(4-Chloro-7-azaindole-2-yl)ethyl)morpholine

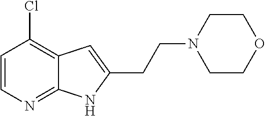 [SMILES: Clc1ccnc2[nH]c(CCN3CCOCC3)cc12]

To a solution of 4-chloro-3-(4-morpholinobut-1-yn-1yl)pyridin-2-amine (0.1 g, 0.37 mmol) in 1,4 dioxane (1 ml), potassium tert-butoxide (0.1 g, 0.94 mmol) and 18-crown-6 (0.009 g, 10%, 0.037 mmol) were added and the mixture was stirred at 120° C. for 18 h. After the reaction was cooled to room temperature, EtOAc and 1M sodium carbonate solution were added. Extracted organic layer was dried over magnesium sulfate and concentrated under reduced pressure to give the product as yellow solid (89.9 mg, 91%), 1H NMR (400 MHz, DMSO-d₆) δ ppm 2.43 (br. s., 4H) 2.67 (t, J=7.69 Hz, 2H) 2.92 (t, J=7.47 Hz, 2H) 3.55-3.60 (m, 4H) 6.28 (s, 1H) 7.12 (d, J=5.27 Hz, 1H) 8.06 (d, J=5.27 Hz, 1H) 11.95 (br. s., 1H). ¹³C NMR (100 MHz, DMSO-d₆) δ ppm 25.75, 53.68, 57.85, 66.75, 95.93, 115.67, 119.92, 132.98, 141.56, 142.48, 149.72. m/z (ESI-MS) [M]⁺ 266.1.

### 5-(2-(2-Morpholinoethyl)-1H-pyrrolo[2,3-b]pyridin-4-yl)-1H-indazol-3-amine

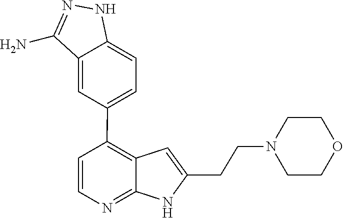 [SMILES: Nc1n[nH]c2ccc(-c3ccnc4[nH]c(CCN5CCOCC5)cc34)cc12]

4-(2-(4-Chloro-7-azaindole-2-yl)ethyl)morpholine (0.06 g, 0.22 mmol), 5-(4,4,5,5-tetramethyl-1,3,2-dioxaborolan-2-yl)-1H-indazol-3-amine (0.08 g, 0.34 mmol), 1M potassium phosphate solution (0.4 ml, 0.4 mmol), [1,1′-bis(di-tert-butylphosphino)ferrocene]dichloropalladium(II) catalyst (0.002 g, 0.01 mmol) in 1.3 ml ethanol were reacted as described in general procedure B and chromatographic purification (90% EtOAc and 1% triethylamine in petroleum ether 60-80%), gave the titled compound as white solid (68.8 mg, 86%). ¹H NMR (400 MHz, DMSO-d₆) b ppm 2.44 (br. s., 4H) 2.69 (t, J=7.47 Hz, 2H) 2.88-2.97 (m, 2H) 3.58 (t, J=4.39 Hz, 4H) 5.52 (s, 2H) 6.48 (s, 1H) 7.12 (d, J=5.27 Hz, 1H) 7.37 (d, J=8.79 Hz, 1H) 7.64 (dd, J=8.79, 0.88 Hz, 1H) 8.13 (s, 1H) 8.15 (d, J=4.83 Hz, 1H) 11.54 (s, 1H) 11.56 (s, 1H). ¹³C NMR (100 MHz, DMSO-d₆) δ ppm 25.95, 53.71, 58.17, 66.76, 97.50, 110.34, 114.46, 115.14, 118.61, 120.70, 127.20, 128.66, 139.99, 140.55, 141.63, 142.28, 149.96, 150.38. m/z (ESI-HRMS) calculated for C₂₀H₂₃ON₆=363.1928 found=363.1924.

### Example 269

### 4-Chloro-3-(5-chloropent-1-yn-1yl)pyridin-2-amine

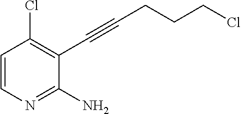 [SMILES: Nc1nccc(Cl)c1C#CCCCCl]

A solution of 4-chloro-3-iodo-pyridine-2-amine (0.15 g, 0.6 mmol), copper (1) iodide (0.005 g, 0.03 mmol) and bis(triphenylphosphine) palladium(II) chloride(0.02 g, 0.03 mmol) in 4 ml of tetrahydrofuran—triethylamine (1:4) was degassed in a sealed tube followed by the addition of 5-chloro-1-pentyne (0.1 ml, 0.9 mmol). The reaction mixture was stirred at room temperature for 16 h. The reaction mixture was diluted with EtOAc and extracted with water. The extracted organic layer was dried over magnesium sulfate and concentrated under reduced pressure.

The crude then purified using column chromatography (10% EtOAc in petroleum ether 60-80%) to give the product as yellow oil (0.102 g, 75%) 1H NMR (400 MHz, DMSO-d₆) δ ppm 2.03 (quin, J=6.70 Hz, 2H) 2.67 (t, J=6.81 Hz, 2H) 3.79 (t, J=6.59 Hz, 2H) 6.48 (br.s, 2H) 6.67 (d, J=5.27 Hz, 1H) 6.84 (d, J=4.83 Hz, 1H). ¹³C NMR (100 MHz, DMSO-d₆) δ ppm 17.48, 31.37, 44.58, 74.36, 100.42, 102.02, 112.80, 144.48, 148.25, 161.55. m/z (ESI-MS) [M]⁺ 229.0.

### 4-Chloro-3-(5-piperdinopent-1-yn-1yl)pyridin-2-amine

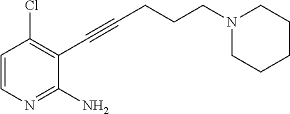 [SMILES: Nc1nccc(Cl)c1C#CCCCN1CCCCC1]

In sealed tube, a mixture of 4-chloro-3-(5-chloropent-1-yn-1yl)pyridin-2-amine (0.1 g, 0.4 mmol), piperdine (0.5 ml) and potassium iodide (0.04 g, 0.22 mmol) in N,N-dimethylamide (1.5 ml) was heated to 110° C. for 50 min. After the reaction was cooled to room temperature, EtOAc and 1 M sodium carbonate solution were added. The extracted organic layer was dried over magnesium sulfate and concentrated under reduced pressure to give the product as yellow oil (0.1 g, 90%), 1H NMR (400 MHz, DMSO-d₆) δ ppm 1.37 (d, J=5.49 Hz, 2H) 1.48 (quin, J=5.49 Hz, 4H) 2.03 (quin, J=7.05 Hz, 2H) 2.31 (br. s., 4H) 2.36 (t, J=7.14 Hz, 2H) 2.67 (t, J=4.61 Hz, 2H) 6.41 (br. s., 2H) 6.66 (dd, J=5.49, 2.93 Hz, 1H) 7.83 (d, J=5.49 Hz, 1H). ¹³C NMR (100 MHz, DMSO-d₆) δ ppm 16.88, 25.58, 30.77, 44.24, 54.10, 57.43, 73.76, 101.61, 102.30, 112.19, 143.70, 147.65, 160.95. m/z (ESI-MS) [M]⁺ 292.1.

### 4-(3-(4-Chloro-7-azaindole-2-yl)propyl)piperidine

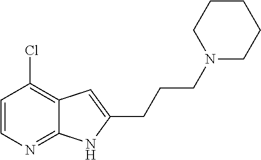 [SMILES: Clc1ccnc2[nH]c(CCCN3CCCCC3)cc12]

To a solution of 4-chloro-3-(5-piperdinopent-1-yn-1yl)pyridin-2-amine (0.1 g, 0.36 mmol) in toluene (1 ml), potassium tert-butoxide (0.1 g, 0.9 mmol) and 18-crown-6 (0.0095 g, 10%, 0.036 mmol) were added and the mixture was stirred at 110° C. for 18 h. After the reaction was cooled to room temperature, EtOAc and 1 M sodium carbonate solution were added. Extracted organic layer was dried over magnesium sulfate and concentrated under reduced pressure to give the product as yellow solid (30 mg, 45%), 1H NMR (400 MHz, DMSO-d₆) δ ppm 1.38 (d, J=5.27 Hz, 2H) 1.43-1.57 (m, 4H) 1.76-1.92 (m, 2H) 2.21-2.40 (m, 6H) 2.75 (t, J=7.69 Hz, 2H) 6.22 (s, 1H) 7.10 (d, J=4.83 Hz, 1H) 8.05 (d, J=5.27 Hz, 1H) 11.97 (br. s. 1H). ¹³C NMR (100 MHz, DMSO-d₆) δ ppm 9.22, 24.75, 26.17, 54.63, 58.63, 70.28, 95.40, 115.73, 119.95, 132.89, 142.38, 143.28, 149.84. m/z (ESI-MS) [M]⁺ 278.2.

### 5-(2-(3-(Piperidin-1-yl)propyl)-1H-pyrrolo[2,3-b]pyridin-4-yl)-1H-indazol-3-amine

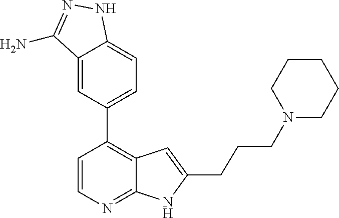 [SMILES: Nc1n[nH]c2ccc(-c3ccnc4[nH]c(CCCN5CCCCC5)cc34)cc12]

4-(3-(4-chloro-7-azaindole-2-yl)propyl)piperidine (0.05 g, 0.22 mmol), 5-(4,4,5,5-tetramethyl-1,3,2-dioxaborolan-2-yl)-1H-indazol-3-amine (0.085 g, 0.33 mmol), 1M potassium phosphate solution (0.44 ml, 0.44 mmol), [1,1′-bis(di-tert-butylphosphino)ferrocene]dichloropalladium(II) catalyst (0.007 g, 0.011 mmol) in 0.45 ml ethanol were reacted as described in general procedure B and purified using HPLC to give the titled compound as yellow solid (0.058 mg, 70%), ¹H NMR (400 MHz, DMSO-d₆) δ ppm 1.48-1.76 (m, 6H) 2.02-2.18 (m, 2H) 2.77-2.95 (m, 4H) 3.01-3.15 (m, 2H) 3.45 (d, J=11.86 Hz, 2H) 6.56 (s, 1H) 7.22 (d, J=5.27 Hz, 1H) 7.44 (d, J=8.79 Hz, 1H) 7.73 (dd, J=8.79, 1.32 Hz, 1H) 8.23 (d, J=6.59 Hz, 2H) 11.54 (br. s., 1H). ¹³C NMR (100 MHz, DMSO-d₆) 24.64, 26.02, 26.56, 31.27, 54.57, 58.69, 96.98, 110.34, 114.41, 115.12, 118.62, 120.68, 127.20, 128.69, 140.44, 141.62, 141.71, 142.17, 150.08, 150.37. m/z (ESI-HRMS) calculated for C₂₂H₂₇N₆=375.2292 found=375.2290.

### Example 270

### 4-Chloro-3-(5-(cyclohexylamino)pent-1-yn-1yl)pyridin-2-amine

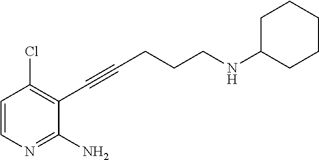 [SMILES: Nc1nccc(Cl)c1C#CCCCNC1CCCCC1]

In sealed tube, a mixture of 4-chloro-3-(5-chloropent-1-yn-1yl)pyridin-2-amine (0.1 g, 0.4 mmol), cyclohexylamine (0.5 ml) and potassium iodide (0.04 g, 0.22 mmol) in N,N-dimethylamide (1.5 ml) was heated to 110° C. for 50 min. After the reaction was cooled to room temperature, EtOAc and 1 M sodium carbonate solution were added. Extracted organic layer was dried over magnesium sulfate and concentrated under reduced pressure to give the product as yellow oil (119 mg, 93%), 1H NMR (400 MHz, DMSO-d₆) δ ppm 1.54 (d, J=10.99 Hz, 2H) 1.60-1.73 (m, 6H) 1.81 (d, J=10.99 Hz, 4H) 2.31-2.43 (m, 1H) 2.56 (t, J=7.03 Hz, 2H) 2.68 (t, J=6.81 Hz, 2H) 6.42 (br. s., 2H) 6.66 (d, J=5.27 Hz, 1H) 7.82 (d, J=5.71 Hz, 1H). ¹³C NMR (100 MHz, DMSO-d₆) δ ppm 17.85, 21.99, 26.43, 29.22, 33.38, 45.67, 56.65, 73.75, 102.26, 102.36, 112.80, 144.25, 148.03, 161.52. m/z (ESI-MS) [M]⁺ 292.1.

### 5-(2-(3-(Cyclohexylamino)propyl)-1H-pyrrolo[2,3-b]pyridin-4-yl)-1H-indazol-3-amine

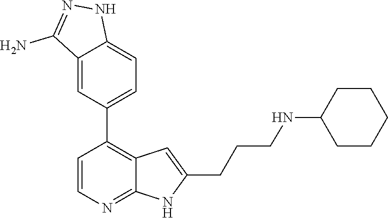 [SMILES: Nc1n[nH]c2ccc(-c3ccnc4[nH]c(CCCNC5CCCCC5)cc34)cc12]

4-(3-(4-Chloro-7-azaindole-2-yl)propyl)cyclohexylamine (0.041 g, 0.14 mmol), 5-(4,4,5,5-tetramethyl-1,3,2-dioxaborolan-2-yl)-1H-indazol-3-amine (0.054 g, 0.21 mmol), 1M potassium phosphate solution (0.28 ml, 0.28 mmol), [1,1′-bis(di-tert-butylphosphino)ferrocene]dichloropalladium(II) catalyst (0.0018 g, 0.007 mmol) in 0.86 ml ethanol were reacted as described in general procedure B and chromatographic purification (18% methanol in EtOAc and 1% triethylamine) gave the titled compound as white solid (38 mg, 69%), ¹H NMR (400 MHz, DMSO-d₆) δ ppm 0.94-1.05 (m, 2H) 1.07-1.24 (m, 4H) 1.53 (dd, J=11.64, 4.17 Hz, 1H) 1.59-1.69 (m, 2H) 1.74-1.89 (m, 4H) 2.31-2.39 (m, 1H) 2.59 (t, J=6.81 Hz, 2H) 2.79 (t, J=7.47 Hz, 2H) 5.51 (s, 2H) 6.43 (s, 1H) 7.11 (d, J=5.27 Hz, 1H) 7.36 (d, J=8.35 Hz, 1H) 7.64 (dd, J=8.57, 1.54 Hz, 1H) 8.08-8.19 (m, 2H) 11.53 (br. s., 1H) 11.62 (br. s., 1H). ¹³C NMR (100 MHz, DMSO-d₆) δ ppm 25.01, 26.45, 26.50, 29.77, 33.45, 46.37, 56.66, 96.94, 110.31, 114.40, 115.14, 118.64, 120.67, 127.19, 128.71, 140.41, 141.63, 141.87, 142.12, 150.05, 150.36. m/z (ESI-HRMS) calculated for C₂₂H₂₇N₇=389.2322 found=389.2319.

### Example 271

### 4-Chloro-3-(5-morpholinopent-1-yn-1yl)pyridin-2-amine

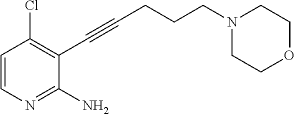 [SMILES: Nc1nccc(Cl)c1C#CCCCN1CCOCC1]

In sealed tube, a mixture of 4-chloro-3-(5-chloropent-1-yn-1yl)pyridin-2-amine (0.1 g, 0.4 mmol), morpholine (0.5 ml) and potassium iodide (0.04 g, 0.22 mmol) in N,N-dimethylamide (1.5 ml) was heated to 110° C. for 50 min. After the reaction was cooled to room temperature, EtOAc and 1 M sodium carbonate solution were added. Extracted organic layer was dried over magnesium sulfate and concentrated under reduced pressure to give the product as yellow oil (115 mg, 90%), 1H NMR (400 MHz, DMSO-d₆) δ ppm 1.73 (quin, J=7.14 Hz, 2H) 2.35 (br. s., 4H) 3.41 (t, J=7.03 Hz, 2H) 2.52-2.56 (m, 2H) 3.57 (t, J=4.61 Hz, 4H) 6.42 (br. s., 2H) 6.66 (d, J=5.71 Hz, 1H) 7.82 (d, J=5.27 Hz, 1H). ¹³C NMR (100 MHz, DMSO-d₆) δ ppm 17.77, 25.57, 53.95, 57.69, 66.77, 79.36, 100.30, 102.30, 112.82, 144.29, 148.05, 161.50. m/z (ESI-MS) [M]⁺ 280.1.

### 4-(3-(4-Chloro-7-azaindole-2-yl)propyl)morpholine

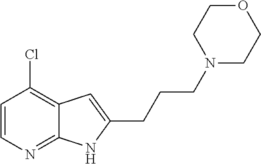 [SMILES: Clc1ccnc2[nH]c(CCCN3CCOCC3)cc12]

To a solution of 4-chloro-3-(5-morpholinopent-1-yn-1yl)pyridin-2-amine (0.1 g, 0.36 mmol) in dioxane (1 ml), potassium tert-butoxide (0.1 g, 0.89 mmol) and 18-crown-6 (0.01 g, 10%, 0.036 mmol) were added and the mixture was stirred at 120° C. for 18 h. After the reaction was cooled to room temperature, EtOAc and 1 M sodium carbonate solution were added. The extracted organic layer was dried over magnesium sulfate and concentrated under reduced pressure to give the product as yellow solid to give the product as yellow solid (100 mg, 99%), 1H NMR (400 MHz, DMSO-d₆) δ ppm 1.73 (quin, J=7.40 Hz, 2H) 2.34 (m, 6H) 2.77 (d, J=7.63 Hz, 2H) 3.58 (t, J=4.58 Hz, 4H) 6.24 (s, 1H) 7.12 (d, J=5.19 Hz, 1H) 8.07 (d, J=5.19 Hz, 1H) 11.95 (br. s., 1H). ¹³C NMR (100 MHz, DMSO-d₆) δ ppm 25.66, 26.12, 53.79, 58.11, 69.97, 95.35, 115.55, 119.83, 132.81, 142.32, 143.08, 149.74. m/z (ESI-MS) [M]⁺ 280.1.

### 5-(2-(3-Morpholinopropyl)-1H-pyrrolo[2,3-b]pyridin-4-yl)-1H-indazlo-3-amine

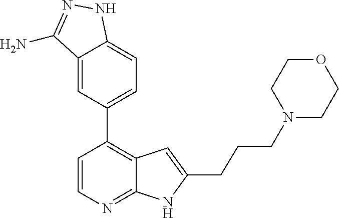 [SMILES: Nc1n[nH]c2ccc(-c3ccnc4[nH]c(CCCN5CCOCC5)cc34)cc12]

4-(3-(4-Chloro-7-azaindole-2-yl)propyl)morpholine (0.09 g, 0.32 mmol), 5-(4,4,5,5-tetramethyl-1,3,2-dioxaborolan-2-yl)-1H-indazol-3-amine (0.12 g, 0.48 mmol), 1M potassium phosphate solution (0.64 ml, 0.64 mmol), [1,1′-bis(di-tert-butylphosphino)ferrocene]dichloropalladium(II) catalyst (0.004 g, 0.016 mmol) in 1.9 ml ethanol were reacted as described in general procedure B and chromatographic purification (90% EtOAc in petroleum ether 60-80%), gave the titled compound as white solid (96.6 mg, 80%), ¹H NMR (400 MHz, DMSO-d₆) δ ppm 1.88 (quin, J=7.36 Hz, 2H) 2.27-2.42 (m, 6H) 2.78 (t, J=7.69 Hz, 2H) 3.57 (t, J=4.61 Hz, 4H) 5.50 (s, 2H) 6.39-6.51 (m, 1H) 7.11 (d, J=4.83 Hz, 1H) 7.37 (d, J=8.79 Hz, 1H) 7.64 (dd, J=8.57, 1.54 Hz, 1H) 8.08-8.23 (m, 2H) 11.53 (s, 1H) 11.58 (s, 1H). ¹³C NMR (100 MHz, DMSO-d₆) b ppm 25.98, 26.40, 53.91, 58.34, 66.77, 97.01, 110.35, 114.43, 115.14, 118.6, 120.68, 127.20, 128.69, 140.46, 141.63, 141.68, 142.18, 150.09, 150.37. m/z (ESI-HRMS) calculated for C₂₁H₂₅ON₆=377.2084 found=377.2082.

### Example 272

### Tert-butyl 4-((2-amino-4-chlorpyridin-3-yl)ethynyl)methyl)piperidine-1-carboxylate

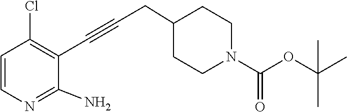 [SMILES: CC(C)(C)OC(=O)N1CCC(CC#Cc2c(Cl)ccnc2N)CC1]

A solution of 4-chloro-3-iodo-pyridine-2-amine (0.12 g, 0.48 mmol), copper (I) iodide (0.004 g, 0.024 mmol) and bis(triphenylphosphine) palladium(II) chloride (0.016 g, 0.024 mmol) in 3 ml of N,N-dimethyformamide-triethylamine (1:4) was degassed in a sealed tube followed by addition of tert-butyl 4-(prop-2-yn-1-yl)piperidine-1-carboxylate (0.13 g, 0.58 mmol). The reaction mixture was stirred at 80° C. for 4 h. The reaction mixture was diluted with EtOAc and extracted with 1 M sodium carbonate. The extracted organic layer was washed with brine and dried over magnesium sulfate and concentrated under reduced pressure and the residue purified by column chromatography (20% EtOAc in petroleum ether 60-80%) to give the product as yellow oil (0.13 g, 77%), ¹H NMR (400 MHz, DMSO-d₆) δ ppm 1.11-1.28 (m, 4H) 1.39 (s, 9H) 1.43-1.45 (m, 1H) 1.74 (d, J=9.67 Hz, 4H) 3.96 (d, J=12.30 Hz, 2H) 6.41 (br. s., 2H) 6.67 (d, J=5.71 Hz, 1H) 7.82 (d, J=5.27 Hz, 1H). ¹³C NMR (100 MHz, DMSO-d₆) δ ppm 26.62, 28.66, 31.33, 35.35, 36.34, 74.92, 79.02, 100.39, 102.25, 112.84, 144.39, 148.11, 154.42, 161.47. m/z (ESI-MS) [M−tert-butyl]⁺ 294.0.

### Tert-butyl 4-((4-chloro-7-azaindol-2-yl)methyl)piperdine-1-carboxylate

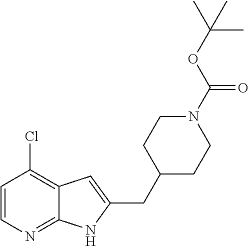 [SMILES: CC(C)(C)OC(=O)N1CCC(Cc2cc3c(Cl)ccnc3[nH]2)CC1]

To a solution of tert-butyl 4-((2-amino-4-chloropyridin-3-yl)ethynyl)methyl)piperidine-1-carboxylate (0.1 g, 0.28 mmol) in 1,4 dioxane (2 ml), potassium tert-butoxide (0.08 g, 0.7 mmol) and 18-crown-6 (0.007 g, 10%, 0.028 mmol) were added and the mixture was stirred at 110° C. for 18 h. After the reaction was cooled to room temperature, EtOAc and 1 M sodium carbonate solution were added. The extracted organic layer was dried over magnesium sulfate and concentrated under reduced pressure to give the product as yellow solid (70 mg, 71%), ¹H NMR (400 MHz, DMSO-d₆) δ ppm 0.95-1.14 (m, 2H) 1.38 (s, 9H) 1.59 (d, J=12.74 Hz, 2H) 1.74 (d, J=12.74 Hz, 2H) 1.80-1.95 (m, 1H) 2.67 (d, J=7.03 Hz, 2H) 3.91 (d, J=13.62 Hz, 2H) 6.24 (s, 1H) 7.12 (d, J=5.27 Hz, 1H) 8.06 (d, J=5.27 Hz, 1H) 11.87 (s, 1H). ¹³C NMR (100 MHz, DMSO-d₆) δ ppm 28.66, 32.10, 35.07, 36.05, 70.30, 79.01, 96.55, 115.71, 119.92, 132.95, 141.21, 142.48, 154.41. m/z (ESI-MS) [M−tert-butyl]⁺ 294.0.

### 2-(Piperdin-4ylmethyl)-4-chloro-7-azaindole

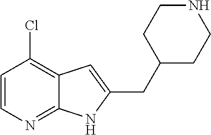 [SMILES: Clc1ccnc2[nH]c(CC3CCNCC3)cc12]

Trifloroacetic acid (0.07 ml) was added to a solution of tert-butyl 4-((4-chloro-7-azaindol-2-yl)methyl)piperdine-1-carboxylate (0.064 g, 0.18 mmol) in dicloromethane (2 ml) and the reaction was stirred at room temperature for 3 h. The reaction mixture was diluted with EtOAc, washed with 1M sodium carbonate, dried over magnesium sulfate and concentrated under vacuum under reduced pressure and the residue purified by column chromatography (80% EtOAc and 1% triethylamine in petroleum ether 60-80%) to give the product as a yellow solid (30 mg, 66%), ¹H NMR (400 MHz, DMSO-d₆) δ ppm 0.90-1.14 (m, 2H) 1.60 (d, J=12.74 Hz, 2H) 1.74 (d, J=12.74 Hz, 2H) 1.80-1.95 (m, 1H) 2.67 (d, J=7.03 Hz, 2H) 3.91 (d, J=13.62 Hz, 2H) 6.62 (s, 1H) 7.20 (d, J=5.27 Hz, 1H) 8.18 (d, J=5.27 Hz, 1H) 12.13 (br. s., 1H). m/z (ESI-MS) [M]⁺ 250.

### 5-(2-(Piperidin-4-ylmethyl)-1H-pyrrolo[2,3-b]pyridin-4-yl)-1H-indazol-3-amine

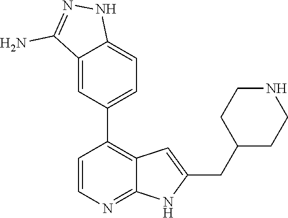 [SMILES: Nc1n[nH]c2ccc(-c3ccnc4[nH]c(CC5CCNCC5)cc34)cc12]

4-Chloro-2-(piperidin-2-ylmethyl)-7-azaindole (0.03 g, 0.086 mmol), 5-(4,4,5,5-tetramethyl-1,3,2-dioxaborolan-2-yl)-1H-indazol-3-amine (0.033 g, 0.13 mmol), 1M potassium phosphate solution (0.17 ml, 0.17 mmol), [1,1′-bis(di-tert-butylphosphino)ferrocene]dichloropalladium(II) catalyst (0.003 g, 0.004 mmol) in 0.5 ml ethanol were reacted as described in general procedure B and HPLC purification gave the titled compound as yellow solid (13.5 mg, 22%), ¹H NMR (400 MHz, DMSO-d₆) δ ppm 1.32-1.45 (m, 2H) 1.79 (d, J=13.28 Hz, 2H) 2.01 (ddd, J=10.76, 7.17, 3.74 Hz, 1H) 2.77 (d, J=6.87 Hz, 2H) 2.79-2.89 (m, 2H) 3.25 (d, J=12.36 Hz, 2H) 6.59 (s, 1H) 7.28 (d, J=5.34 Hz, 1H) 7.49 (d, J=8.70 Hz, 1H) 7.79 (d, J=8.70 Hz, 1H) 8.27 (d, J=5.34 Hz, 1H) 8.29 (s, 1H) 8.60 (d, J=9.31 Hz, 1H) 12.02 (br. s., 1H). ¹³C NMR (100 MHz, DMSO-d₆) δ ppm 28.68, 33.94, 34.53, 43.62, 98.85, 111.50, 114.49, 115.48, 119.58, 121.50, 128.67, 128.89, 139.97, 140.19, 141.85, 141.89, 158.57, 158.85. m/z (ESI-HRMS) calculated for C₂₀H₂₃N₆=347.1979 found=347.1977.

### Example 273

### 5-(2-((1-benzylpiperidin-4-yl)methyl)-1H-pyrrolo[2,3-b]pyridin-4-yl)-1H-indazol-3-amine

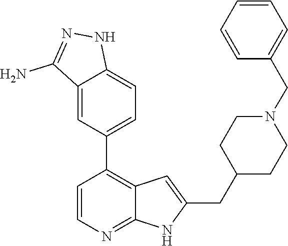 [SMILES: Nc1n[nH]c2ccc(-c3ccnc4[nH]c(CC5CCN(Cc6ccccc6)CC5)cc34)cc12]

To a solution of 5-(2-(piperdin-4-ylmethyl)-7-azaindol-4-yl)-1H-indazol-3-amine (0.012 g, 0.034 mmol) and potassium carbonate (0.0046 g, 0.034 mmol) in a N,N-dimethylformamide (1 ml) at 100° C., chloromethylbenzene (0.0048 ml, 0.038 mmol) was added and the reaction mixture allowed to stirred for 2 h. then, the reaction mixture concentrated and the residue purified by HPLC to give the title compound as yellow solid (0.84 mg, 8%), ¹H NMR (400 MHz, DMSO-d₆) δ ppm 1.37-1.50 (m, 2H) 1.79-1.88 (m, 2H) 1.89-2.00 (m, 1H) 2.73 (d, J=6.59 Hz, 2H) 3.33 (d, J=10.99 Hz, 4H) 4.26 (d, J=4.39 Hz, 2H) 6.49 (s, 1H) 7.19 (d, J=5.27 Hz, 1H) 7.41 (d, J=8.79 Hz, 1H) 7.46 (s, 5H) 7.70 (dd, J=8.79, 1.32 Hz, 1H) 8.18 (s, 1H) 8.21 (d, J=5.27 Hz, 1H) 11.78 (br. s., 1H). m/z (ESI-HRMS) calculated for C₂₇H₂₉N₆=437.2448 found=437.2453.

### Section 12—Compounds of the Formula

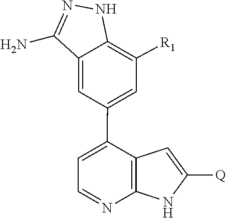

### 3-Chloro-2-fluoro-5-(4,4,5,5-tetramethyl-1,3,2-dioxaborolan-2-yl)benzonitrile

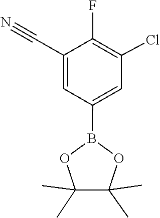 [SMILES: CC1(C)OB(c2cc(Cl)c(F)c(C#N)c2)OC1(C)C]

Preparation of stock solution: [Ir(OMe)cod]₂ ([(1,5-cyclooctadiene) (methoxy) iridium (I)dimer]) (104 mg, 0.312 mmol), dtbpy (di-tert-butyl-2,2′dipyridyl) (84 mg, 0.312 mmol) and B₂pin₂ (bis (pinacolato) diboron) (2644 mg, 10.4 mmol) were mixed in volumetric flask and diluted up to 25 ml with MTBE (tert-butyl-methylether). Then 12.6 ml of stock solution was added to 3-chloro-2-fluorobenzonitrile (0.85 g, 5.5 mmol) in microwave vial under nitrogen. The reaction vial then deoxygenated, sealed and heated to 80° C. for 30 min in microwave. Thereafter, the solvent was evaporated and residue purified by column chromatography (0-20% EtOAc in petroleum ether 60-80%) to give the title compound as a white solid (1.3 g, 84%). ¹H NMR (400 MHz, DMSO-d₆) δ ppm 1.31 (s, 12H) 7.99-8.07 (m, 2H). ¹³C NMR (100 MHz, DMSO-d₆) δ ppm 25.05, 85.32, 102.93, 113.26, 121.36, 136.54, 138.60, 141.51, 159.40.

### 7-Chloro-5-(4,4,5,5-tetramethyl-1,3,2-dioxaborolan-2-yl)-1H-indazol-3-amine

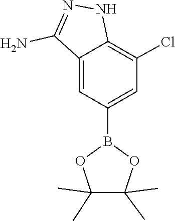 [SMILES: CC1(C)OB(c2cc(Cl)c3[nH]nc(N)c3c2)OC1(C)C]

To a solution of 3-chloro-2-fluoro-5-(4,4,5,5-tetramethyl-1,3,2-dioxaborolan-2-yl) benzonitrile (187) (0.4 g, 1.4 mmol) in ethanol (20 ml), hydrazine hydrate (0.22 ml, 2.47 g, 6.8 mmol, 50-60%) was added and the reaction refluxed for 18 h. The solvent was evaporated and the residue was triturated with 12 ml EtOAc and petroleum ether (1:1) and then filtrated under vacuum and washed with water and petroleum ether 60-80% to give the desired compound as a yellow solid (320 mg, 78%). ¹H NMR (400 MHz, DMSO-d₆) δ ppm 1.31 (s, 12H) 5.68 (s, 2H) 7.45 (s, 1H) 8.15 (s, 1H) 12.02 (br. s., 1H). ¹³C NMR (100 MHz, DMSO-d₆) δ ppm 25.17, 84.07, 102.89, 116.70, 119.82, 125.89, 128.07, 130.59, 146.33.

### 3-(3,3-Dimethylbut-1-yn-1-yl)-2-fluorobenzonitrile

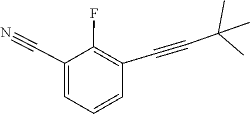

To a solution of 3-bromo-2-fluorobenzonitrile (1 g, 5 mmol), copper (1) iodide (0.047 g, 0.25 mmol) and bis(triphenylphosphine) palladium(II) chloride (0.175 g, 0.25 mmol) in 15 ml of N,N-dimethyformamide-triethylamine (1:4) was degassed in sealed tube followed by 3,3-dimethylbut-1-yne (0.61 g, 7.5 mmol). The reaction mixture was stirred at 80° C. for 3 h. After reaction completed, the reaction mixture was diluted with EtOAc and extract with 1 M sodium carbonate. Extracted organic layer was washed with brine and dried over magnesium sulfate and concentrated under reduced pressure and the residue purified by column chromatography (5% EtOAc in petroleum ether 60-80%) to give the product as yellow solid (1 g, 99%). ¹H NMR (400 MHz, DMSO-d₆) δ ppm 01.31 (s, 9H) 7.38 (t, J=7.69 Hz, 1H) 7.80 (td, J=7.58, 1.54 Hz, 1H) 7.90 (ddd, J=7.80, 6.26, 1.76 Hz, 1H).

### Potassium (phenyleth-1-yn-1-yl) trifluoroborate

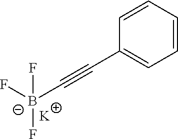 [SMILES: F[B-](F)(F)C#Cc1ccccc1 ; [K+]]

To a solution of ethynylbenzene (1.6 ml, 15 mmol) in tetrahydrofuran (30 ml) at −78° C., n-butyllithium (6 ml, 15 mmol, 2.5 M) was added gradually and the solution was stirred for 1 h at this temperature. Then, trimethylborate (2.5 ml, 22.5 mmol) was added and the reaction mixture allowed to stirred at −78° C. for 1 h and then warm to −20° C. and stirred for another hour. Saturated aqueous solution of potassium hydrogen difluoride (7 g, 90 mmol) was added and reaction mixture allowed to warm to room temperature and stirred for 18 h. The solvent then evaporated and the residue washed with acetone and filtered. The organic layer then concentrated and the residue dissolved in hot acetone and precipitated with diethyl ether. The solid then collected to give the title compound as white solid (2.8 g, 90%). ¹H NMR (400 MHz, DMSO-d₆) δ ppm 7.28 (m, 3H) 7.28-7.29 (m, 2H). ¹³C NMR (100 MHz, DMSO-d₆) δ ppm 125.53, 126.67, 128.22, 130.90.

### General Procedure a:

A suspension of required chloroaryl (1 eq.), 5-(4,4,5,5-tetramethyl-1,3,2-dioxaborolan-2-yl)-1H-indazol-3-amine (1.2-1.5 eq.), base (2 eq.) in 1:3 of solvent was deoxygenated with nitrogen in sealed tube. Then [1,1′-bis(di-tert-butylphosphino)ferrocene]dichloropalladium(II) catalyst (0.05 eq.) was added then the tube was sealed and the mixture allowed to stirred at 90-100° C. for 18 h. After the reaction was cooled to room temperature, EtOAc and water were added. Extracted organic layer was dried over magnesium sulfate and concentrated under reduced pressure and the residue purified by column chromatography.

### Example 274

### 4-(3-Amino-7-chloro-1H-indazol-5-yl)-N-(2-(piperidin-1-yl)ethyl)-1H-pyrrolo[2,3-b]pyridine-2-carboxamide

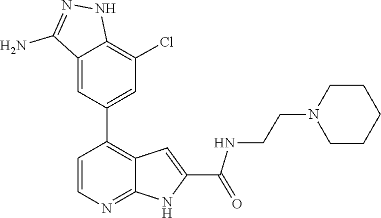 [SMILES: Nc1n[nH]c2c(Cl)cc(-c3ccnc4[nH]c(C(=O)NCCN5CCCCC5)cc34)cc12]

A 2-5 mL Biotage MW tube was charged with 4-chloro-N-(2-(piperidin-1-yl)ethyl)-1H-pyrrolo[2,3-b]pyridine-2-carboxamide (100 mg, 0.3 mmol), 7-chloro-5-(4,4,5,5-tetramethyl-1,3,2-dioxaborolan-2-yl)-1H-indazol-3-amine (111 mg, 0.4 mmol, 1.3 eq.), [1,1′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over magnesium carbonate filtered and the solvent removed under reduced pressure and the crude product was then purified by HPLC to give the desired product as a pale yellow solid (54 mg). ¹H NMR (500 MHz, DMSO-D₆) δ 1.36-1.48 (m, 7H), 2.36-2.53 (m, 5H) 3.39 (s, 2H), 5.70 (s, 2H), 7.23 (d, J=5.05 Hz, 1H), 7.33 (d, J=1.7 Hz, 1H), 7.69 (s, 1H), 8.12 (s, 1H), 8.37 (d, J=5.00 Hz, 1H), 8.47 (bs, 1H), 12.07 (s, 1H), 12.18 (s, 1H). LRMS: Calculated for C₂₂H₂₄ClN₇O 437.17; Found: 438.20

### Example 275

### 7-Chloro-5-(2-(cyclohexylmethyl)-1H-pyrrolo[2,3-b]pyridin-4-yl)-1H-indazol-3-amine

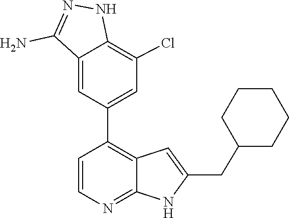 [SMILES: Nc1n[nH]c2c(Cl)cc(-c3ccnc4[nH]c(CC5CCCCC5)cc34)cc12]

4-Chloro-2-(cyclohexylmethyl)-7-azaindole (0.08 g, 0.34 mmol), 7-chloro-5-(4,4,5,5-tetramethyl-1,3,2-dioxaborolan-2-yl)-1H-indazol-3-amine (0.15 g, 0.51 mmol), 1M potassium phosphate solution (0.68 ml, 0.68 mmol), [1,1′-bis(di-tert-butylphosphino)ferrocene]dichloropalladium(II) catalyst (0.007 g, 0.01 mmol) in 2 ml ethanol were reacted as described in General procedure A and purified by column chromatography (60% EtOAc and 1% triethylamnie in petroleum ether 60-80%) to give the titled compound as white solid (81 mg, 63%), ¹H NMR (400 MHz, DMSO-d₆) δ ppm 0.92-1.03 (m, 2H) 1.10-1.18 (m, 4H) 1.16 (d, J=9.46 Hz, 1H) 1.66 (d, J=10.83 Hz, 4H) 2.65 (d, J=7.02 Hz, 2H) 5.69 (s, 2H) 6.40 (s, 1H) 7.14 (d, J=4.88 Hz, 1H) 7.67 (s, 1H) 8.14 (d, J=1.37 Hz, 1H) 8.16 (d, J=4.88 Hz, 1H) 11.56 (s, 1H) 12.03 (s, 1H). ¹³C NMR (100 MHz, DMSO-d₆) 26.10, 26.44, 33.12, 36.18, 37.93, 97.45, 111.92, 114.42, 116.92, 118.46, 119.71, 125.97, 128.73, 129.89, 138.75, 140.89, 141.98, 149.89, 151.16. m/z (ESI-HRMS) calculated for C₂₁H₂₃N₅CI=380.1636 found=380.1641.

### Example 276

### 7-Chloro-5-(2-(morpholinomethyl)-1H-pyrrolo[2,3-b]pyridin-4-yl)-1H-indazol-3-amine

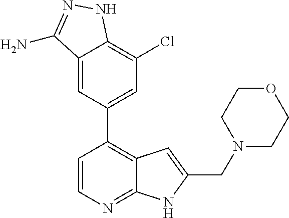 [SMILES: Nc1n[nH]c2c(Cl)cc(-c3ccnc4[nH]c(CN5CCOCC5)cc34)cc12]

4-((4-Chloro-7-azaindole-2-yl)methyl)morpholine (0.1 g, 0.36 mmol), 7-chloro-5-(4,4,5,5-tetramethyl-1,3,2-dioxaborolan-2-yl)-1H-indazol-3-amine (0.16 g, 0.53 mmol), 1M potassium phosphate solution (0.72 ml, 0.72 mmol), [1,1′-bis(di-tert-butylphosphino)ferrocene]dichloropalladium(II) catalyst (0.008 g, 0.01 mmol) in 2.2 ml ethanol were reacted as described in General procedure A and purified by column chromatography (90% EtOAc and 1% triethylamine in petroleum ether 60-80%) to give the titled compound as white solid (65 mg, 47%), ¹H NMR (400 MHz, DMSO-d₆) δ ppm 2.42 (br. s., 4H) 3.58 (t, J=4.17 Hz, 4H) 3.66 (s, 2H) 5.75 (br. s., 2H) 6.57 (d, J=1.76 Hz, 1H) 7.17 (d, J=4.83 Hz, 1H) 7.68 (d, J=1.32 Hz, 1H) 8.15 (d, J=0.88 Hz, 1H) 8.21 (d, J=4.83 Hz, 1H) 11.77 (s, 1H) 12.08 (s, 1H). ¹³C NMR (100 MHz, DMSO-d₆) 46.20, 53.61, 66.59, 99.19, 113.17, 114.61, 116.90, 118.04, 118.50, 119.84, 122.96, 126.00, 129.67, 139.61, 142.89, 147.45, 149.92. m/z (ESI-MS) [M]⁺ 383.2.

### Example 277

### 7-Chloro-5-(2-(2-morpholinoethyl)-1H-pyrrolo[2,3-b]pyridin-4-yl)-1H-indazol-3-amine

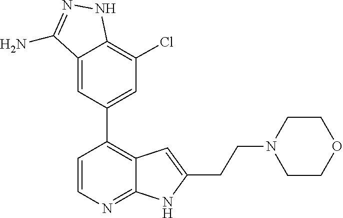 [SMILES: Nc1n[nH]c2c(Cl)cc(-c3ccnc4[nH]c(CCN5CCOCC5)cc34)cc12]

4-(2-(4-Chloro-7-azaindole-2-yl)ethyl)morpholine (0.1 g, 0.37 mmol), 7-chloro-5-(4,4,5,5-tetramethyl-1,3,2-dioxaborolan-2-yl)-1H-indazol-3-amine (0.16 g, 0.56 mmol), 1M potassium phosphate solution (0.74 ml, 0.74 mmol), [1,1′-bis(di-tert-butylphosphino)ferrocene]dichloropalladium(II) catalyst (0.008 g, 0.01 mmol) in 2.2 ml ethanol were reacted as described in general procedure and purified by column chromatography (90% EtOAc and 1% triethylamnie in petroleum ether 60-80%) to give the titled compound as white solid (63.3 mg, 43%), ¹H NMR (400 MHz, DMSO-d₆) δ ppm 2.45 (br. s., 4H) 2.70 (t, J=7.71 Hz, 2H) 2.94 (t, J=7.63 Hz, 2H) 3.59 (t, J=4.58 Hz, 4H) 5.69 (br. s., 2H) 6.38-6.54 (m, 1H) 7.15 (d, J=5.04 Hz, 1H) 7.68 (d, J=1.22 Hz, 1H) 8.11-8.21 (m, 2H) 11.61 (s, 1H) 12.03 (br. s., 1H). ¹³C NMR (100 MHz, DMSO-d₆) 25.84, 53.60, 58.02, 66.68, 97.14, 110.82, 114.46, 116.17, 118.42, 119.73, 125.96, 126.99, 129.81, 138.95, 140.37, 142.18, 149.86, 150.58. m/z (ESI-HRMS) calculated for C₂₀H₂₂ON₆Cl=397.1538 found=397.1542.

### Example 278

### 5-(2-(Cyclohexylmethyl)-1H-pyrrolo[2,3-b]pyridin-4-yl)-7-(3,3-dimethylbut-1-yn-1-yl)-1H-indazol-3-amine

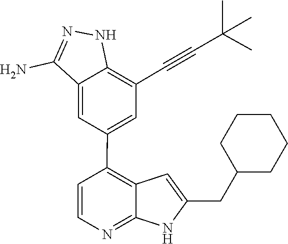 [SMILES: CC(C)(C)C#Cc1cc(-c2ccnc3[nH]c(CC4CCCCC4)cc23)cc2c(N)n[nH]c12]

A solution of 7-chloro-5-(2-(cyclohexylmethyl)-7-azaindol-4-yl)-1H-indazol-3-amine (0.03 g, 0.08 mmol), chloro (2-dicyclohexylphosphino-2′,4′,6′-triisopropyl-1,1′-biphenyl) [2-(2′-amino-1,1′-biphenyl)]palladium(II) (0.003 g, 0.004 mmol), 2 M potassium carbonate (0.23 ml, 0.4 mmol) and potassium (3,3-dimethylbut-1-yn-1-yl) trifluoroborate (0.03 g, 0.158 mmol) in 0.7 ml of 1,4 dioxane was degassed in a sealed tube and stirred at 110° C. for 18 h. Thereafter, the reaction mixture was diluted with EtOAc and extract with 1 M sodium carbonate. The extracted organic layer was washed with brine, dried over magnesium sulfate and concentrated under reduced pressure and the residue purified by HPLC to give the product as yellow solid (13.3 mg, 26%), 1H NMR (400 MHz, DMSO-d₆) δ ppm 0.93-1.04 (m, 2H) 1.07-1.32 (m, 4H) 1.38 (s, 9H) 1.67 (d, J=11.80 Hz, 4H) 1.74 (td, J=6.40, 3.51 Hz, 1H) 2.68 (d, J=7.03 Hz, 2H) 6.49 (s, 1H) 7.27 (d, J=5.02 Hz, 1H) 7.62 (d, J=1.25 Hz, 1H) 8.20 (m, 2H) 11.88 (br. s., 1H) 12.01 (br. s., 1H). m/z (ESI-HRMS) calculated for C₂₇H₃₂N₅₌426.2652 found=426.2657.

### Example 279

### 7-(3,3-Dimethylbut-1-yn-1-yl)-5-(2-(morpholinomethyl)-1H-pyrrolo[2,3-b]pyridin-4-yl)-1H-indazol-3-amine

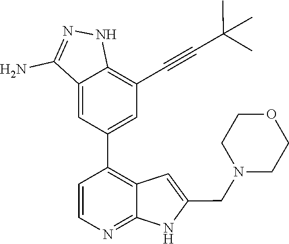 [SMILES: CC(C)(C)C#Cc1cc(-c2ccnc3[nH]c(CN4CCOCC4)cc23)cc2c(N)n[nH]c12]

A solution of 7-chloro-5-((2-morpholinomethyl)-7-azaindol-4-yl)-1H-indazol-3-amine (0.03 g, 0.07 mmol), chloro (2-dicyclohexylphosphino-2′,4′,6′-triisopropyl-1,1′-biphenyl) [2-(2′-amino-1,1′-biphenyl)]palladium (II) (0.005 g, 0.007 mmol), 2 M potassium carbonate (0.21 ml, 0.42 mmol) and potassium (3,3-dimethylbut-1-yn-1-yl) trifluoroborate (0.03 g, 0.157 mmol) in 0.7 ml of dioxane was degassed in a sealed tube and stirred at 110° C. for 18 h. Thereafter, the reaction mixture was diluted with EtOAc and extract with 1 M sodium carbonate. The extracted organic layer was washed with brine, dried over magnesium sulfate and concentrated under reduced pressure and the residue purified by HPLC to give the product as yellow solid (10.7 mg, 23%), 1H NMR (400 MHz, DMSO-d₆) δ ppm 1.37 (s, 9H) 3.15 (br. s., 2H) 3.38 (br. s., 2H) 3.64 (br. s., 2H) 3.95 (br. s., 2H) 4.53 (br. s., 2H) 6.90 (s, 1H) 7.27 (d, J=4.83 Hz, 1H) 7.59 (d, J=1.32 Hz, 1H) 8.16 (d, J=1.32 Hz, 1H) 8.35 (d, J=4.83 Hz, 1H) 11.92 (br. s., 1H) 12.00 (br. s., 1H). ¹³C NMR (100 MHz, DMSO-d₆) δ ppm 31.20, 31.24, 51.63, 53.13, 63.94, 74.86, 95.67, 98.45, 103.99, 106.08, 115.35, 117.88, 121.11, 121.27, 128.25, 129.42, 138.56, 141.97, 142.67, 149.71, 150.81. m/z (ESI-MS) [M]⁺ 429.1.

### Example 280

### 7-(3,3-Dimethylbut-1-yn-1-yl)-5-(2-(2-morpholinoethyl)-1H-pyrrolo[2,3-b]pyridin-4-yl)-1H-indazol-3-amine

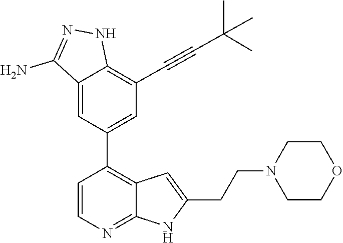 [SMILES: CC(C)(C)C#Cc1cc(-c2ccnc3[nH]c(CCN4CCOCC4)cc23)cc2c(N)n[nH]c12]

A solution of 7-chloro-5-(2-(2-morpholinoethyl)-7-azaindol-4-yl)-1H-indazol-3-amine (0.03 g, 0.075 mmol), chloro (2-dicyclohexylphosphino-2′,4′,6′-triisopropyl-1,1′-biphenyl) [2-(2′-amino-1,1′-biphenyl)]palladium(II) (0.006 g, 0.007 mmol), 2 M potassium carbonate (0.22 ml, 0.45 mmol) and potassium (3,3-dimethylbut-1-yn-1-yl) trifluoroborate (0.03 g, 0.151 mmol) in 0.7 ml of 1,4 dioxane was degassed in a sealed tube and stirred at 110° C. for 18 h. Thereafter, the reaction mixture was diluted with EtOAc and extract with 1 M sodium carbonate. The extracted organic layer was washed with brine, dried over magnesium sulfate and concentrated under reduced pressure and the residue purified by HPLC to give the product as yellow solid (10.4 mg, 17%), 1H NMR (400 MHz, DMSO-d₆) δ ppm 1.38 (s, 9H) 3.16 (br. s., 2H) 3.25 (t, J=7.78 Hz, 2H) 3.45-3.60 (m, 4H) 3.67 (t, J=11.29 Hz, 2H) 4.02 (d, J=12.05 Hz, 2H) 6.58 (s, 1H) 7.20 (d, J=5.02 Hz, 1H) 7.57 (d, J=1.51 Hz, 1H) 8.13 (d, J=1.51 Hz, 1H) 8.24 (d, J=5.02 Hz, 1H) 11.79-11.97 (m, 2H). m/z (ESI-HRMS) calculated for C₂₆H₃₁ON₆=443.2554 found=443.2557.

### Example 281

### 5-(2-(2-Morpholinoethyl)-1H-pyrrolo[2,3-b]pyridin-4-yl)-7-(phenylethynyl)-1H-indazol-3-amine

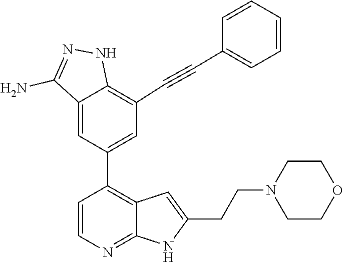 [SMILES: Nc1n[nH]c2c(C#Cc3ccccc3)cc(-c3ccnc4[nH]c(CCN5CCOCC5)cc34)cc12]

A solution of 7-chloro-5-(2-(2-morpholinoethyl)-7-azaindol-4-yl)-1H-indazol-3-amine (0.03 g, 0.075 mmol), chloro (2-dicyclohexylphosphino-2′,4′,6′-triisopropyl-1,1′-biphenyl) [2-(2′-amino-1,1′-biphenyl)]palladium(II) (0.006 g, 0.007 mmol), 2 M potassium carbonate solution (0.22 ml, 0.45 mmol) and potassium (phenyleth-1-yn-1-yl) trifluoroborate (193) (0.03 g, 0.151 mmol) in 0.7 ml of 1,4 dioxane was degassed in a sealed tube and stirred at 110° C. for 18 h. Thereafter, the reaction mixture was diluted with EtOAc and extract with 1 M sodium carbonate. The extracted organic layer was washed with brine, dried over magnesium sulfate and concentrated under reduced pressure and the residue purified by HPLC to give the product as yellow solid (17 mg, 16%), 1H NMR (400 MHz, DMSO-d₆) δ ppm 3.15 (br. s., 2H) 3.27 (t, J=7.91 Hz, 2H) 3.42-3.61 (m, 4H) 3.68 (t, J=10.92 Hz, 2H) 3.90-4.09 (m, 2H) 6.64 (s, 1H) 7.27 (d, J=5.27 Hz, 1H) 7.44-7.51 (m, 3H) 7.72-7.76 (m, 2H) 7.82 (d, J=1.51 Hz, 1H) 8.25 (d, J=1.51 Hz, 1H) 8.28 (d, J=5.02 Hz, 1H) 11.95 (br. s., 1H). m/z (ESI-HRMS) calculated for C₂₈H₂₇ON₈=463.2241 found=463.2245.

### Example 294

### 5-(2-Cyclopropyl-1H-pyrrolo[2,3-b]pyridin-4-v)-7-(3,3-dimethylbut-1-yn-1-Vl)-1H-indazol-3-amine

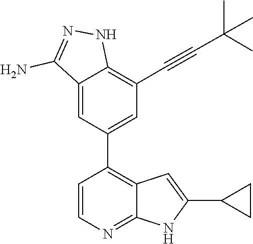 [SMILES: CC(C)(C)C#Cc1cc(-c2ccnc3[nH]c(C4CC4)cc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (94.6 mg, 0.279 mmol), 4-chloro-2-cyclopropyl-1H-pyrrolo[2,3-b]pyridine (53.7 mg, 0.279 mmol), Pd(dtbpf)Cl₂ (18.2 mg, 0.028 mmol), and caesium carbonate (273 mg, 0.836 mmol) under an argon atmosphere. To the vial was added dioxane:water (9:1) and the mixture was degassed the stirred at 80° C. for 18 hours. The mixture was cooled to room temperature, diluted with ethyl acetate and saturated sodium carbonate solution, and the organic phase separated. The aqueous phase was extracted with ethyl acetate three times, and the combined organic phase was dried (magnesium sulfate), filtered, and concentrated in vacuo. Flash column chromatography [petrol:ethyl acetate 0→100%] followed by preparative HPLC purification afforded the title compound as an orange solid (11.8 mg, 11%). ¹H NMR (400 MHz, DMSO-d₆) δ 11.76 (br s, 1H, Ar—NH), 11.49 (br s, 1H, Ar—NH), 8.14-8.06 (m, 2H, 2xAr—H), 7.53 (d, J=1.4 Hz, 1H, Ar—H), 7.09 (d, J=4.9 Hz, 1H, Ar—H), 6.36 (d, J=1.8 Hz, 1H, Ar—H), 5.61 (br s, 2H, NH₂), 2.13-2.03 (m, 1H, ° Pr—CH), 1.37 (s, 9H, ^{t}Bu), 1.03-0.95 (m, 2H, ° Pr—CH₂), 0.92-0.85 (m, 2H, ° Pr—CH₂). LRMS: Calculated for C₂₃H₂₃N₅ 369.20 found 370.3 (M+1).

### Route to Example 294

### 4-Chloro-3-(cyclopropylethynyl)pyridin-2-amine

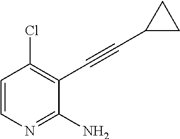 [SMILES: Nc1nccc(Cl)c1C#CC1CC1]

To a degassed solution of 4-chloro-3-iodopyridin-2-amine (300 mg, 1.18 mmol), Pd₂Cl₂(PPh₃)₂(82.8 mg, 0.118 mmol), and copper(I) iodide (44.9 mg, 0.236 mmol) in 1:1 N,N-dimethylformamide: triethylamine (0.3 mM) was added cyclopropylacetylene (162 μL, 2.36 mmol) and the mixture was stirred at 60° C. for 16 hours. The mixture was cooled to room temperature, filtered over celite, diluted with ethyl acetate and brine, and the organic phase separated. The aqueous phase was extracted with ethyl acetate three times and the combined organic phase was dried (magnesium sulfate), filtered, and concentrated in vacuo. Flash column chromatography [petrol:ethyl acetate 0→30%] afforded the title compound as an orange oil that solidified upon standing (216 mg, 1.12 mmol, 95%). ¹H NMR (400 MHz, CDCl₃) δ 7.82 (d, J=5.5 Hz, 1H, Ar—H), 6.66 (d, J=5.5 Hz, 1H, Ar—H), 5.10 (br s, 2H, NH₂), 1.59-1.50 (m, 1H, ° Pr—CH), 0.98-0.90 (m, 2H, ° Pr—CH₂), 0.90-0.84 (m, 2H, ° Pr—CH₂). LRMS: Calculated for C₁₀H₉³⁵ClN₂ 192.05 found 193.1 (M+1).

### 4-Chloro-2-cyclopropyl-1H-pyrrolo[2,3-b]pyridine

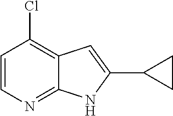 [SMILES: Clc1ccnc2[nH]c(C3CC3)cc12]

To a solution of 4-chloro-3-(cyclopropylethynyl)pyridin-2-amine (151 mg, 0.783 mmol) and 18-crown-6 (20.7 mg, 0.0783 mmol) in dry toluene (65 mM) was added potassium tert-butoxide (862 μL, 1.72 mmol, 2M in tetrahydrofuran) and the mixture was stirred at 100° C. for 18 hours. The mixture was cooled to room temperature, diluted with ethyl acetate and saturated sodium carbonate solution, and the organic phase separated. The aqueous phase was extracted with ethyl acetate three times and the combined organic phase was dried (magnesium sulfate), filtered, and concentrated in vacuo. Flash column chromatography [petrol:ethyl acetate 0→40%] afforded the title compound as pale yellow solid (22.8 mg, 0.118 mmol, 15%). ¹H NMR (500 MHz, CDCl₃) δ 11.14 (br s, 1H, NH), 8.12 (d, J=5.3 Hz, 1H, Ar—H), 7.06 (d, J=5.3 Hz, 1H, Ar—H), 6.25 (d, J=1.5 Hz, 1H, Ar—H), 2.13-2.06 (m, 1H, ° Pr—CH), 1.13-1.09 (m, 2H, ° Pr—CH₂), 0.98-0.93 (m, 2H, ° Pr—CH₂). LRMS: Calculated for C₁₀H₉³⁵ClN₂ 192.05 found 193.1 (M+1).

### Example 295

### 5-(2-Cyclopentyl-1H-pyrrolo[2,3-b]pyridin-4-yl)-7-(3,3-dimethylbut-1-yn-1-yl)-1H-indazol-3-amine

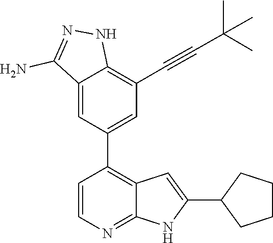 [SMILES: CC(C)(C)C#Cc1cc(-c2ccnc3[nH]c(C4CCCC4)cc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (88.6 mg, 0.261 mmol), 4-chloro-2-cyclopentyl-1H-pyrrolo[2,3-b]pyridine (57.6 mg, 0.261 mmol), Pd(dtbpf)Cl₂ (17.0 mg, 0.026 mmol), and caesium carbonate (255 mg, 0.783 mmol) under an argon atmosphere. To the vial was added dioxane:water (9:1) and the mixture was degassed the stirred at 80° C. for 18 hours. The mixture was cooled to room temperature, diluted with ethyl acetate and saturated sodium carbonate solution, and the organic phase separated. The aqueous phase was extracted with ethyl acetate three times, and the combined organic phase was dried (magnesium sulfate), filtered, and concentrated in vacuo. Flash column chromatography [petrol:ethyl acetate 0→100%] followed by preparative HPLC purification afforded the title compound as a brown solid (10.7 mg, 10%). ¹H NMR (400 MHz, DMSO-d₆) δ 11.78 (br s, 1H, Ar—NH), 11.58 (br s, 1H, Ar—NH), 8.14 (d, J=5.0 Hz, 1H, Ar—H), 8.12 (d, J=1.4 Hz, 1H, Ar—H), 7.53 (d, J=1.4 Hz, 1H, Ar—H), 7.10 (d, J=5.0 Hz, 1H, Ar—H), 6.38 (d, J=1.4 Hz, 1H, Ar—H), 5.62 (br s, 2H, NH₂), 3.19 (pent, J=8.0 Hz, 1H, ° Pent-CH), 2.14-2.03 (m, 2H, ° Pent-CH₂), 1.83-1.60 (m, 6H, ° Pent-CH₂), 1.36 (s, 9H, ^{t}Bu). LRMS: Calculated for C₂₅H₂₇N₅ 397.23 found 398.3 (M+1).

### Route to Example 295

### 4-Chloro-3-(cyclopentylethynyl)pyridin-2-amine

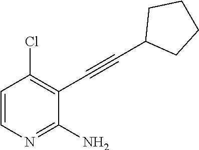 [SMILES: Nc1nccc(Cl)c1C#CC1CCCC1]

To a degassed solution of 4-chloro-3-iodopyridin-2-amine (300 mg, 1.18 mmol), Pd₂Cl₂(PPh₃)₂(82.8 mg, 0.118 mmol), and copper(I) iodide (44.9 mg, 0.236 mmol) in 1:1 N,N-dimethylformamide: triethylamine (0.3 mM) was added cyclopentylacetylene (284 μL, 2.36 mmol) and the mixture was stirred at 60° C. for 16 hours. The mixture was cooled to room temperature, filtered over celite, diluted with ethyl acetate and brine, and the organic phase separated. The aqueous phase was extracted with ethyl acetate three times and the combined organic phase was dried (magnesium sulfate), filtered, and concentrated in vacuo. Flash column chromatography [petrol:ethyl acetate 0→20%] afforded the title compound as an orange oil that solidified upon standing (255 mg, 1.16 mmol, 98%). ¹H NMR (500 MHz, CDCl₃) δ 7.78 (d, J=5.5 Hz, 1H, Ar—H), 6.63 (d, J=5.6 Hz, 1H, Ar—H), 5.28 (br s, 2H, NH₂), 2.96-2.86 (m, 1H, CH), 2.01-1.95 (m, 2H, CH₂), 1.82-1.68 (m, 6H, CH₂), 1.65-1.55 (m, 2H, CH₂). LRMS: Calculated for C₁₂H₁₃³⁵ClN₂ 220.08 found 221.2 (M+1).

### 4-Chloro-2-cyclopentyl-1H-pyrrolo[2,3-b]pyridine

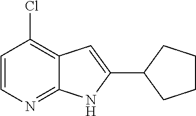 [SMILES: Clc1ccnc2[nH]c(C3CCCC3)cc12]

To a solution of 4-chloro-3-(cyclopentylethynyl)pyridin-2-amine (230 mg, 1.04 mmol) in dioxane (4 mL) was added potassium tert-butoxide (293 mg, 2.61 mmol) and 18-crown-6 (27.6 mg, 0.104 mmol) and the mixture was stirred at 100° C. for 18 hours. The mixture was cooled to room temperature, diluted with ethyl acetate and saturated sodium carbonate solution, and the organic phase separated. The aqueous phase was extracted with ethyl acetate three times and the combined organic phase was dried (magnesium sulfate), filtered, and concentrated in vacuo. Trituration from 1:2 diethyl ether:petroleum ether afforded the title compound as a pale brown solid (61.0 mg, 0.276 mmol, 27%). The filtrate was resubjected to the above conditions using 18-crown-6 (15 mg, 0.0567 mmol) and potassium tert-butoxide (700 μL, 1.40 mmol, 2M in tetrahydrofuran) under microwave irradiation at 130° C. for 30 minutes. Flash column chromatography of the combined filtrand and resubjected filtrate [petrol:ethyl acetate 0→40%] afforded the title compound as a pale yellow solid (92.6 mg, 0.420 mmol, 40%). ¹H NMR (500 MHz, CDCl₃) δ 10.86 (br s, 1H, NH), 8.09 (d, J=5.3 Hz, 1H, Ar—H), 7.07 (d, J=5.3 Hz, 1H, Ar—H), 6.32 (d, J=1.6 Hz, 1H, Ar—H), 3.34-3.21 (m, 1H, CH), 2.25-2.13 (m, 2H, CH₂), 1.92-1.70 (m, 6H, CH₂). LRMS: Calculated for CO₁₂H₁₃³⁵ClN₂ 220.08 found 221.2 (M+1).

### Example 296

### 5-(2-(tert-Butyl)-1H-pyrrolo[2,3-b]pyridin-4-yl)-7-(3,3-dimethylbut-1-yn-1-yl)-1H-indazol-3-amine

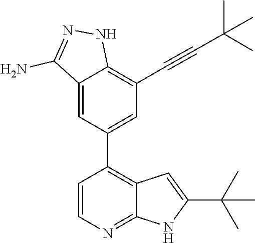 [SMILES: CC(C)(C)C#Cc1cc(-c2ccnc3[nH]c(C(C)(C)C)cc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (93.3 mg, 0.275 mmol), 2-(tert-butyl)-4-chloro-1H-pyrrolo[2,3-b]pyridine (52.2 mg, 0.250 mmol), Pd(dtbpf)Cl₂ (16.3 mg, 0.025 mmol), and caesium carbonate (163 mg, 0.500 mmol) under an argon atmosphere. To the vial was added DMF:water (4:1) and the mixture was degassed the stirred at 100° C. for 18 hours. The mixture was cooled to room temperature, diluted with ethyl acetate and saturated sodium carbonate solution, and the organic phase separated. The aqueous phase was extracted with ethyl acetate three times, and the combined organic phase was washed with lithium chloride solution (10 w/v %) five times, dried (magnesium sulfate), filtered, and concentrated in vacuo. Flash column chromatography [petrol:ethyl acetate 0→100%] followed by preparative HPLC purification afforded the title compound as a white solid (13.5 mg, 14%). ¹H NMR (400 MHz, DMSO-d₆) δ 11.78 (br s, 1H, NH), 11.63 (br s, 1H, NH), 8.16 (d, J=5.0 Hz, 1H, Ar—H), 8.13 (s, 1H, Ar—H), 7.54 (s, 1H, Ar—H), 7.10 (d, J=5.0 Hz, 1H, Ar—H), 6.32 (s, 1H, Ar—H), 5.63 (br s, 2H, NH₂), 1.38 (s, 9H, ^{t}Bu), 1.36 (s, 9H, ^{t}Bu). LRMS: Calculated for C₂₄H₂₇N₅ 385.23 found 386.3 (M+1).

### Route to Example 296

### 4-Chloro-3-(3,3-dimethylbut-1-yn-1-yl)pyridin-2-amine

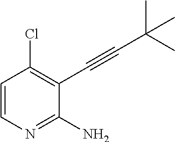

To a degassed solution of 4-chloro-3-iodopyridin-2-amine (300 mg, 1.18 mmol), Pd₂Cl₂(PPh₃)₂(82.8 mg, 0.118 mmol), and copper(I) iodide (44.9 mg, 0.236 mmol) in 1:1 N,N-dimethylformamide: triethylamine (0.3 mM) was added 3,3-dimethyl-1-butyne (290 μL, 2.36 mmol) and the mixture was stirred at 60° C. for 16 hours. The mixture was cooled to room temperature, filtered over celite, diluted with ethyl acetate and brine, and the organic phase separated. The aqueous phase was extracted with ethyl acetate three times and the combined organic phase was dried (magnesium sulfate), filtered, and concentrated in vacuo. Flash column chromatography [petrol:ethyl acetate 0→30%] afforded the title compound as a yellow solid (246 mg, 1.18 mmol, quant). ¹H NMR (500 MHz, CDCl₃) δ 7.83 (d, J=5.4 Hz, 1H, Ar—H), 6.67 (d, J=5.4 Hz, 1H, Ar—H), 5.07 (br s, 2H, NH₂), 1.36 (s, 9H, ^{t}Bu). LRMS: Calculated for C₁₁H₁₃³⁵ClN₂ 208.08 found 209.2 (M+1).

### 2-(tert-Butyl)-4-chloro-1H-pyrrolo[2,3-b]pyridine

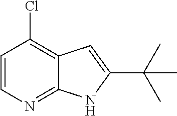

To a solution of 4-chloro-3-(3,3-dimethylbut-1-yn-1-yl)pyridin-2-amine (236 mg, 1.13 mmol) and 18-crown-6 (29.9 mg, 0.113 mmol) in dry toluene (65 mM) was added potassium tert-butoxide (1.25 mL, 2.49 mmol, 2M in tetrahydrofuran) and the mixture was stirred at 100° C. for 18 hours. The mixture was cooled to room temperature, diluted with ethyl acetate and saturated sodium carbonate solution, and the organic phase separated. The aqueous phase was extracted with ethyl acetate three times and the combined organic phase was dried (magnesium sulfate), filtered, and concentrated in vacuo. Flash column chromatography [petrol:ethyl acetate 0→40%] afforded the title compound as a pale yellow solid (65.3 mg, 0.313 mmol, 28%). ¹H NMR (500 MHz, CDCl₃) δ 10.99 (br s, 1H, NH), 8.15 (d, J=5.3 Hz, 1H, Ar—H), 7.08 (d, J=5.3 Hz, 1H, Ar—H), 6.32 (d, J=2.3 Hz, 1H, Ar—H), 1.48 (s, 9H, ^{t}Bu). LRMS: Calculated for C₁₁H₁₃ClN₂ 208.08 found 209.2 (M+1).

### Example 297

### (4-(3-Amino-7-(3,3-dimethylbut-1-yn-1-yl)-1H-indazol-5-yl)-1H-pyrrolo[2,3-b]pyridin-2-yl)methanol

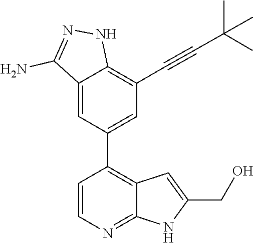 [SMILES: CC(C)(C)C#Cc1cc(-c2ccnc3[nH]c(CO)cc23)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (56.72 mg, 0.167 mmol, 1.1 Eq) in a degassed 9:1 solution of dioxane/water (3 mL), were added (4-chloro-1H-pyrrolo[2,3-b]pyridin-2-yl)methanol (27.77 mg, 0.152 mmol, 1 Eq), cesium carbonate (148.57 mg, 0.456 mmol, 3 Eq) and 1,1′-bis(di-tert-butylphosphino) ferrocene palladium chloride (9.90 mg, 0.0152 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a white solid (41 mg, 0.114 mmol, 75%).

¹H NMR (500 MHz, DMSO-d₆) δ 11.78 (s, 1H), 11.60 (s, 1H), 8.18 (d, J=5.0 Hz, 1H), 8.13 (d, J=1.7 Hz, 1H), 7.56 (d, J=1.5 Hz, 1H), 7.14 (d, J=5.0 Hz, 1H), 6.54 (d, J=2.0 Hz, 1H), 5.60 (s, 2H), 5.27 (t, J=5.6 Hz, 1H), 4.65 (d, J=5.7 Hz, 2H), 1.37 (s, 9H) ppm. LRMS: Calculated for C₂₁H₂₁N₅O requires 359.43 found 360.3 (M+H).

### Route to Example 297

### (4-Chloro-1H-pyrrolo[2,3-b]pyridin-2-yl)methanol

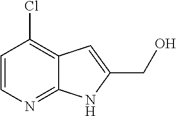 [SMILES: OCc1cc2c(Cl)ccnc2[nH]1]

To a solution of methyl 4-chloro-1H-pyrrolo[2,3-b]pyridine-2-carboxylate (300 mg, 1.42 mmol, 1 Eq) in 2 mL of tetrahydrofuran was added dropwise lithium aluminium hydride 1M solution in THF (0.5 mL, 5.68 mmol, 4 Eq) at room temperature under argon atmosphere. The resulting reaction mixture was heated to 70° C. for 3h.

The reaction was then cooled to 0° C., EtOAc (10 mL) and water (10 mL) were added sequentially, and the resulting suspension was filtered through celite. The filtrate was washed with EtOAc (10 mL) and the organic and aqueous layers were separated. The aqueous layer was extracted with EtOAc (2×10 mL). The combined organic layer was washed with water (2×10 mL), dried over anhydrous sodium sulphate, filtered and evaporated under reduced pressure to obtain the titled compound (129.65 mg, 0.71 mmol, 50%) which was sufficiently pure for the next step. ¹H NMR (500 MHz, DMSO-d₆) δ 11.91 (s, 1H), 8.12-8.06 (m, 1H), 7.16-7.11 (m, 1H), 6.36 (s, 1H), 5.36 (t, J=5.5 Hz, 1H), 4.62 (d, J=4.6 Hz, 2H) ppm. LRMS (ESI+ve): Calculated for C₈H₇ClN₂O requires 182.6 found 183.1 (M+H).

### Example 298

### 2-(4-(3-Amino-7-(3,3-dimethylbut-1-yn-1-yl)-1H-indazol-5-yl)-1H-pyrrolo[2,3-b]pyridin-2-yl)propan-2-ol

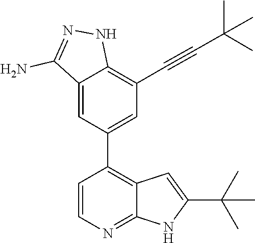 [SMILES: CC(C)(C)C#Cc1cc(-c2ccnc3[nH]c(C(C)(C)C)cc23)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (56.04 mg, 0.165 mmol, 1.1 Eq) in a degassed 9:1 solution of dioxane/water (3 mL), were added 2-(4-chloro-1H-pyrrolo[2,3-b]pyridin-2-yl)propan-2-ol (32 mg, 0.150 mmol, 1 Eq), cesium carbonate (146.61 mg, 0.45 mmol, 3 Eq) and 1,1′-bis(di-tert-butylphosphino) ferrocene palladium chloride (9.77 mg, 0.0150 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a white solid (30 mg, 0.114 mmol, 55%).

¹H NMR (500 MHz, DMSO-d₆) δ 11.77 (s, 1H), 11.52 (br. s, 1H), 8.17 (d, J=5.0 Hz, 1H), 8.11 (d, J=1.6 Hz, 1H), 7.54 (d, J=1.5 Hz, 1H), 7.11 (d, J=5.0 Hz, 1H), 6.45 (d, J=2.1 Hz, 1H), 5.61 (s, 2H), 5.20 (s, 1H), 1.56 (s, 6H), 1.37 (s, 9H) ppm. LRMS: Calculated for C₂₃H₂₅N₅O requires 387.49 found 388.3 (M+H).

### Route to Example 298

### 2-(4-Chloro-1H-pyrrolo[2,3-b]pyridin-2-yl)propan-2-ol

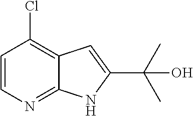

To a solution of methyl 4-chloro-1H-pyrrolo[2,3-b]pyridine-2-carboxylate (300 mg, 1.42 mmol, 1 Eq) in 1.5 mL of tetrahydrofuran was added dropwise a solution of 2.5M methylmagnesium bromide in diethyl ether (1.14 mL, 2.84 mmol, 2 Eq) at 0° C. under argon atmosphere. The resulting reaction mixture was then allowed to stir at 40° C. overnight.

The reaction was poured slowly into an ice-cooled saturated solution of NH4Cl and extracted with EtOAc (10 mL). The combined organic layer was washed with brine (2×10 mL), dried over anhydrous sodium sulphate, filtered and evaporated under reduced pressure. The residue was purified by flash column chromatography (60% EtOAc in petroleum ether) to afford the titled compound (269.22 mg, 1.28 mmol, 90%). ¹H NMR (500 MHz, DMSO-d₆) δ 11.84 (s, 1H), 8.09 (d, J=5.2 Hz, 1H), 7.12 (d, J=5.2 Hz, 1H), 6.30 (d, J=1.8 Hz, 1H), 5.29 (s, 1H), 3.32 (s, 1H), 1.54 (s, 6H) ppm. LRMS (ESI+ve): Calculated for C₁₀H₁₁ClN₂O requires 210.6 found 211.2 (M+H).

### Example 299

### Methyl 4-(3-amino-7-(3,3-dimethylbut-1-yn-1-yl)-1H-indazol-5-yl)-1H-pyrrolo[2,3-b]pyridine-2-carboxylate

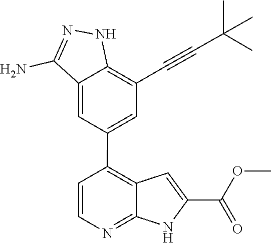 [SMILES: COC(=O)c1cc2c(-c3cc(C#CC(C)(C)C)c4[nH]nc(N)c4c3)ccnc2[nH]1]

To a solution of 7-(3,3-dimethylbut-1-yn-1-yl)-5-(4,4,5,5-tetramethyl-1,3,2-dioxaborolan-2-yl)-1H-indazol-3-amine (354.14 mg, 1.044 mmol, 1.1 Eq) in a degassed 9:1 solution of dioxane/water (5 mL), were added methyl 4-chloro-1H-pyrrolo[2,3-b]pyridine-2-carboxylate (200 mg, 0.949 mmol, 1 Eq), cesium carbonate (927.61 mg, 2.847 mmol, 3 Eq) and 1,1′-bis(di-tert-butylphosphino) ferrocene palladium chloride (61.85 mg, 0.0949 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a white solid (73.53 mg, 0.189 mmol, 20%).

¹H NMR (500 MHz, DMSO-d₆) δ 12.63 (s, 1H), 11.84 (s, 1H), 8.44 (d, J=4.8 Hz, 1H), 8.20 (d, J=1.5 Hz, 1H), 7.56 (d, J=1.5 Hz, 1H), 7.35 (d, J=2.1 Hz, 1H), 7.29 (d, J=4.9 Hz, 1H), 5.69 (br. s, 2H), 3.90 (s, 3H), 1.37 (s, 9H) ppm. LRMS: Calculated for C₂₂H₂₁N₅O₂ requires 387.44 found 388.1 (M+H).

### Section 13—Imidazopyridines

### Example 300

### 5-(2-(Difluoromethyl)-3H-imidazo[4,5-b]pyridin-7-yl)-7-(3,3-dimethylbut-1-yn-1-yl)-1H-indazol-3-amine

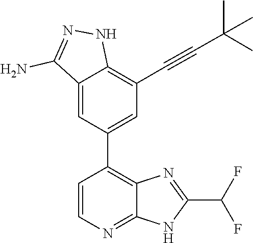 [SMILES: CC(C)(C)C#Cc1cc(-c2ccnc3[nH]c(C(F)F)nc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (125 mg, 0.368 mmol), 7-chloro-2-(difluoromethyl)-3H-imidazo[4,5-b]pyridine (75.0 mg, 0.368 mmol), Pd(dtbpf)Cl₂ (24.0 mg, 0.0368 mmol), and caesium carbonate (360 mg, 1.11 mmol) under an argon atmosphere. To the vial was added DMF:water (9:1) and the mixture was degassed the stirred at 100° C. for 18 hours. The mixture was cooled to room temperature, diluted with ethyl acetate and saturated sodium bicarbonate solution, and the organic phase separated. The aqueous phase was extracted with ethyl acetate three times, and the combined organic phase was washed with lithium chloride solution (10 w/v %) five times, dried (magnesium sulfate), filtered, and concentrated in vacuo. Flash column chromatography [petrol:ethyl acetate 0→100%] followed by preparative HPLC purification afforded the title compound as a yellow solid (11.7 mg, 8%). ¹H NMR (400 MHz, DMSO-d₆) δ 12.91 (br s, 1H, NH), 11.78 (br s, 1H, NH), 8.53 (d, J=1.5 Hz, 1H, Ar—H), 8.42 (d, J=1.5 Hz, 1H, Ar—H), 8.25 (d, J=5.2 Hz, 1H, Ar—H), 7.44 (d, J=5.2 Hz, 1H, Ar—H), 5.58 (br s, 2H, NH₂), 3.79 (p, J=8.7 Hz, 1H, ^{c}Bu-CH), 2.48-2.31 (m, 4H, ^{c}Bu-CH₂), 2.14-2.02 (m, 1H, ^{c}Bu-CH₂), 2.02-1.88 (m, 1H, ^{c}Bu-CH₂), 1.37 (s, 9H, ^{t}Bu). LRMS: Calculated for C₂₀H₁₈F₂N₆ 380.16 found 381.3 (M+1).

### Route to Example 300

### 7-Chloro-2-(difluoromethyl)-3H-imidazo[4,5-b]pyridine

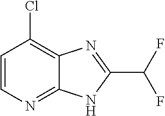 [SMILES: FC(F)c1nc2c(Cl)ccnc2[nH]1]

A solution of 4-chloropyridine-2,3-diamine (144 mg, 1.00 mmol) and carboxylic acid (70 μL, 1.1 mmol) in polyphosphoric acid (4 mL) was stirred at 100° C. for 18 hours in a sealed microwave vial. The mixture was cooled to room temperature, diluted with a water and ice, and adjusted to pH7 (NaOH pellets followed by 2M NaOH solution). The mixture was extracted with ethyl acetate three times and the combined organic phase was dried (magnesium sulfate), filtered, and concentrated in vacuo to afford the title compound as a pale brown solid (166 mg, 81%), which was used without further purification. ¹H NMR (400 MHz, DMSO-d₆) δ 14.38 (br s, 1H, NH), 8.41 (d, J=5.2 Hz, 1H, Ar—H), 7.51 (d, J=5.2 Hz, 1H, Ar—H), 7.32 (t, J=52.9 Hz, 1H, CF₂H). LRMS: Calculated for C₇H₄³⁵CIF₂N₃ 203.01 found 204.1 (M+1).

### Example 301

### 5-(2-Cyclobutyl-3H-imidazo[4,5-b]pyridin-7-yl)-7-(3,3-dimethylbut-1-yn-1-yl)-1H-indazol-3-amine

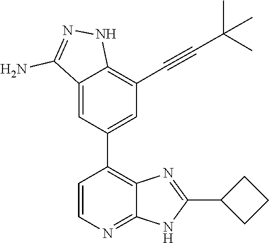 [SMILES: CC(C)(C)C#Cc1cc(-c2ccnc3[nH]c(C4CCC4)nc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (97.1 mg, 0.286 mmol), 7-chloro-2-cyclobutyl-3H-imidazo[4,5-b]pyridine (65.4 mg, 0.315 mmol), Pd(dtbpf)Cl₂ (25.0 mg, 0.0384 mmol), and caesium carbonate (280 mg, 0.859 mmol) under an argon atmosphere. To the vial was added DMF:water (9:1) and the mixture was degassed the stirred at 100° C. for 18 hours. The mixture was cooled to room temperature, diluted with ethyl acetate and saturated sodium carbonate solution, and the organic phase separated. The aqueous phase was extracted with ethyl acetate three times, and the combined organic phase was washed with lithium chloride solution (10 w/v %) five times, dried (magnesium sulfate), filtered, and concentrated in vacuo. Flash column chromatography [petrol:ethyl acetate 0→100%] followed by preparative HPLC purification afforded the title compound as a white solid with a ca. 15% impurity (5.5 mg, 5%). ¹H NMR (400 MHz, DMSO-d₆) δ 12.91 (br s, 1H, NH), 11.78 (br s, 1H, NH), 8.53 (d, J=1.5 Hz, 1H, Ar—H), 8.42 (d, J=1.5 Hz, 1H, Ar—H), 8.25 (d, J=5.2 Hz, 1H, Ar—H), 7.44 (d, J=5.2 Hz, 1H, Ar—H), 5.58 (br s, 2H, NH₂), 3.79 (p, J=8.7 Hz, 1H, ^{c}Bu-CH), 2.48-2.31 (m, 4H, ^{c}Bu-CH₂), 2.14-2.02 (m, 1H, ^{c}Bu-CH₂), 2.02-1.88 (m, 1H, ^{c}Bu-CH₂), 1.37 (s, 9H, ^{t}Bu). LRMS: Calculated for C₂₃H₂₄N₆ 384.21 found 385.4 (M+1).

### Route to Example 301

### 7-Chloro-2-cyclobutyl-3H-imidazo[4,5-b]pyridine

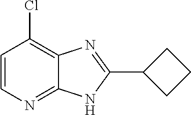 [SMILES: Clc1ccnc2[nH]c(C3CCC3)nc12]

A solution of 4-chloropyridine-2,3-diamine (144 mg, 1.00 mmol) and carboxylic acid (105 μL, 1.1 mmol) in polyphosphoric acid (4 mL) was stirred at 130° C. for 18 hours in a sealed microwave vial. The mixture was cooled to room temperature, diluted with a water and ice, and adjusted to pH7 (NaOH pellets followed by 2M NaOH solution). The title compound was collected via vacuum filtration as a black solid (211 mg, 100%), which was used without further purification. ¹H NMR (500 MHz, DMSO-d₆) δ 13.15 (br s, 1H, NH), 8.16 (d, J=5.3 Hz, 1H, Ar—H), 7.27 (d, J=5.3 Hz, 1H, Ar—H), 3.74 (p, J=8.6 Hz, 1H, ^{c}Bu-CH), 2.47-2.39 (m, 2H, ^{c}Bu-CH₂), 2.38-2.30 (m, 2H, ^{c}Bu-CH₂), 2.12-2.01 (m, 1H, ^{c}Bu-CH₂), 1.96-1.88 (m, 1H, ^{c}Bu-CH₂). LRMS: Calculated for C₁₀H₁₀₃₅ClN₃ 207.06 found 208.2 (M+1).

### Example 308

### 7-(3,3-dimethylbut-1-yn-1-yl)-5-(2-(trifluoromethyl)-3H-imidazo[4,5-b]pyridin-7-yl)-1H-indazol-3-amine

### Route to Example 308

### 7-Chloro-2-(trifluoromethyl)-3H-imidazo[4,5-b]pyridine

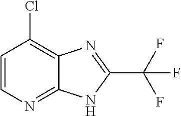 [SMILES: FC(F)(F)c1nc2c(Cl)ccnc2[nH]1]

A solution of 4-chloropyridine-2,3-diamine (144 mg, 1.00 mmol) and trifluoroacetic acid (80 μL, 1 mmol) in polyphosphoric acid (4 mL) was stirred at 100° C. for 18 hours in a sealed microwave vial. The mixture was cooled to room temperature, diluted with a water and ice, and adjusted to pH7 (NaOH pellets followed by 2M NaOH solution). The mixture was extracted with ethyl acetate three times and the combined organic phase was dried (magnesium sulfate), filtered, and concentrated in vacuo to afford the title compound as a pale brown solid (187 mg, 85%), which was used without further purification. ¹H NMR (500 MHz, DMSO-d₆) δ 15.09 (br s, 1H, NH), 8.50 (d, J=5.3 Hz, 1H, Ar—H), 7.60 (d, J=5.3 Hz, 1H, Ar—H). LRMS: Calculated for C₇H₃CIF₃N₃ 221.57 found 222.1 (M+1).

### 7-(3,3-Dimethylbut-1-yn-1-yl)-5-(2-(trifluoromethyl)-3H-imidazo[4,5-b]pyridin-7-yl)-1H-indazol-3-amine SU1737

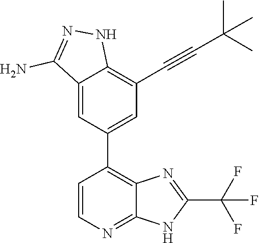 [SMILES: CC(C)(C)C#Cc1cc(-c2ccnc3[nH]c(C(F)(F)F)nc23)cc2c(N)n[nH]c12]

To a microwave vial was added 7-(3,3-dimethylbut-1-yn-1-yl)-5-(4,4,5,5-tetramethyl-1,3,2-dioxaborolan-2-yl)-1H-indazol-3-amine (123 mg, 0.361 mmol), 7-chloro-2-(trifluoromethyl)-3H-imidazo[4,5-b]pyridine (80 mg, 0.361 mmol), Pd(dtbpf)Cl₂ (24 mg, 0.036 mmol), and caesium carbonate (353 mg, 1.08 mmol) under an argon atmosphere. To the vial was added DMF:water (9:1) and the mixture was degassed the stirred at 100° C. for 18 hours. The mixture was cooled to room temperature, diluted with ethyl acetate and saturated sodium bicarbonate solution, and the organic phase separated. The aqueous phase was extracted with ethyl acetate three times, and the combined organic phase was washed with lithium chloride solution (10 w/v %) five times, dried (magnesium sulfate), filtered, and concentrated in vacuo. Preparative HPLC purification followed by recrystallisation from ethyl acetate afforded the title compound as a yellow solid (47 mg, 33%). ¹H NMR (500 MHz, DMSO-d₆) δ 14.78 (br s, 1H, NH), 11.90 (br s, 1H, NH), 8.58 (s, 1H, Ar—H), 8.52 (d, J=4.8 Hz, 1H, Ar—H), 8.32 (s, 1H, Ar—H), 7.66 (d, J=4.8 Hz, 1H, Ar—H), 5.66 (br s, 2H, NH₂), 1.38 (s, 9H, ^{t}Bu). LRMS: Calculated for C₂₀H₁₇F₃N₆ 398.4 found 399.3 (M+1).

### Additional Examples

### General Procedures

### General Procedure 1: Suzuki Coupling

To a microwave vial was added aryl chloride (1-1.1 equiv.), aryl BPin (1-1.1 equiv.), Palladium catalyst [10 mol %], and cesium carbonate (2-3 equiv.) under an argon atmosphere. To the vial was added dioxane:water or DMF:water (9:1-4:1) and the mixture was degassed the stirred at 80° C. for 18 hours or 120° C. for 90 minutes under microwave irradiation. The mixture was cooled to room temperature, diluted with ethyl acetate and saturated sodium carbonate solution, and the organic phase separated. The aqueous phase was extracted with ethyl acetate three times, if DMF was used, the organic phase was extracted with LiCl 10% w/v solution five times, and the combined organic phase was dried (magnesium sulfate), filtered, and concentrated in vacuo.

### General Procedure 2: Buchwald Coupling

To a microwave vial under inert atmosphere was added Pd(OAc)₂ [2-5 mol %] and Xantphos [3-10 mol %], followed by dry solvent which was stirred at r.t.—80° C. for 10 minutes to afford the catalyst solution. To a separate microwave vial under inert atmosphere was added aryl halide (1 equiv.), amine (1-1.5 equiv.), and base (1-2 equiv.), followed by solvent and catalyst solution. The mixture was degassed and stirred at 60-110° C. for 18 hours. The mixture was cooled to room temperature, diluted with ethyl acetate and saturated sodium carbonate solution, and the organic phase separated. The aqueous phase was extracted with ethyl acetate three times, and the combined organic phase was dried (magnesium sulfate), filtered, and concentrated in vacuo.

### General Procedure 3: Tetrahydropyran Protection

To a round bottomed flask was added amine (1 equiv.) and PTSA [10 mol %] under an inert atmosphere. Dry THF followed by 3,4-dihydro-2H-pyran (2 equiv.) was added and the reaction was stirred at 65° C. for 18 hours. The mixture was cooled to room temperature, diluted with ethyl acetate and water, and the organic phase separated. The aqueous phase was extracted with ethyl acetate three times, and the combined organic phase was dried (magnesium sulfate), filtered, and concentrated in vacuo.

### General Procedure 4: Nitro Reduction

To a round bottomed flask was added nitro compound (1 equiv.) and palladium on carbon (10%, wet) [10 mol %] in methanol. The flask was purged with hydrogen gas for 5 minutes, then stirred at room temperature for 5 hours. The mixture was allowed to cool and concentrated in vacuo.

### Example 309—N-(4-(3-Amino-7-(3,3-dimethylbut-1-yn-1-yl)-1H-indazol-5-yl)pyridin-2-yl)cyanamide SU1770

### Route to SU1770

### N¹-Hydroxy-N-(4-iodopyridin-2-yl)formimidamide

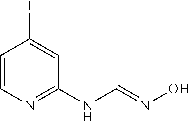 [SMILES: O/N=C/Nc1cc(I)ccn1]

N,N-Dimethylformamide dimethyl acetal (1.6 g, 1.8 mL, 13.64 mmol, 3 Eq) was added to a solution of 4-iodopyridin-2-amine (1 g, 4.55 mmol, 1 Eq) in 2-propanol (10 mL). The reaction mixture was refluxed for 2 h under a nitrogen. The reaction mixture was cooled to 50° C. and hydroxylamine hydrochloride (0.6 g, 9.1 mmol, 2 Eq) was added and the reaction mixture was allowed to stir at 50° C. for 18 h under nitrogen. The reaction mixture was concentrated under reduced pressure and the residue was allowed to stir in a mixture DCM (20 mL) and NaOH aqueous solution (2N, 5 mL) for 30 min. The organic layer was dried over anhydrous sodium sulfate and removed under reduced pressure. The crude residue was purified using column chromatography (50% EtOAc in petroleum ether) to afford the titled compound as white solid (0.42 g, 1.6 mmol, 35%).

¹H NMR (500 MHz, DMSO) δ 10.20 (s, 1H), 9.43 (d, J=9.9 Hz, 1H), 7.85 (d, J=5.3 Hz, 1H), 7.81 (d, J=9.8 Hz, 1H), 7.53 (s, 1H), 7.21 (d, J=5.3 Hz, 1H).

### N-(4-Iodopyridin-2-yl)cyanamide

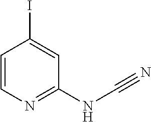

POCl₃ (0.7 g, 0.4 mL, 4.56 mmol, 1.2 Eq) was added dropwise to a solution of N¹-hydroxy-N-(4-iodopyridin-2-yl)formimidamide (1 g, 3.8 mmol, 1 Eq) in anhydrous DMA (5 mL) at 0° C. while purged with nitrogen. The reaction mixture was allowed to stir under nitrogen at rt for 2 h. The reaction mixture was extracted between EtOAc (20 mL) and NaOH aqueous solution (1N, 5 mL). The organic layer was dried over anhydrous sodium sulfate and removed under reduced pressure. The crude residue was purified using column chromatography (5% MeOH in EtOAc) to afford the titled compound as white solid (0.47 g, 1.9 mmol, 50%).

¹H NMR (500 MHz, DMSO) δ 4.11 (s, 1H), 6.60 (dd, J=5.2, 1.5 Hz, 1H), 6.73 (d, J=1.6 Hz, 1H), 7.53 (d, J=5.3 Hz, 1H).

### N-(4-(3-Amino-7-(3,3-dimethylbut-1-yn-1-yl)-1H-indazol-5-yl)pyridin-2-yl)cyanamide SU1770

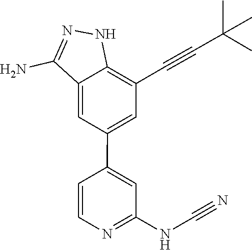 [SMILES: CC(C)(C)C#Cc1cc(-c2ccnc(NC#N)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (66 mg, 0.2 mmol, 1.2 Eq), N-(4-iodopyridin-2-yl)cyanamide (40 mg, 0.16 mmol, 1 Eq), cesium carbonate (156 mg, 0.48 mmol, 3 Eq) and bis(triphenylphosphine)palladium chloride (17 mg, 0.03 mmol, 0.15 Eq) in dioxane (2 mL) and water (0.5 mL) was stirred under nitrogen atmosphere at 80° C. for 18 h. The solvents were removed under reduced pressure. The crude residue was purified using column chromatography (10% MeOH in EtOAc) followed by trituration using MeOH to give the titled product as yellow solid (10 mg, 0.03 mmol, 20%).

¹H NMR (500 MHz, DMSO) δ 11.96 (s, 1H), 8.28 (s, 1H), 7.82 (d, J=6.4 Hz, 1H), 7.59 (s, 1H), 7.17 (s, 1H), 7.05 (d, J=6.7 Hz, 1H), 5.74 (s, 2H), 1.38 (s, 9H).

LCMS: For C₁₉H₁₈N₆ requires 330.40 found 331.3 (M+H).

### Example 310—5-(2-((1H-Pyrazol-3-yl)amino)pyridin-4-yl)-7-(3,3-dimethylbut-1-yn-1-yl)-1H-indazol-3-amine SU1759

### Route to SU1759

### 3-Nitro-1-(tetrahydro-2H-pyran-2-yl)-1H-pyrazole

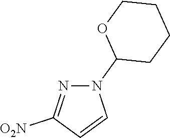 [SMILES: O=[N+]([O-])c1ccn(C2CCCCO2)n1]

General procedure 3 was followed using 3-nitro-1H-pyrazole (113.1 mg, 1 mmol), PTSA (17.2 mg, 0.1 mmol), and dihydropyran (183 μL, 2 mmol). Flash column chromatography [petrol:ethyl acetate 0→30%] afforded the title compound (197 mg, quant).

¹H NMR (500 MHz, DMSO-d₆) δ 8.19 (d, J=2.6 Hz, 1H), 7.09 (d, J=2.6 Hz, 1H), 5.57 (dd, J=9.6, 2.5 Hz, 1H), 3.97-3.89 (m, 1H), 3.71-3.63 (m, 1H), 2.12-2.02 (m, 1H), 2.01-1.89 (m, 2H), 1.74-1.62 (m, 2H), 1.60-1.49 (m, 2H).

¹³C NMR (101 MHz, DMSO-d₆) δ 155.16, 132.77, 102.85, 87.63, 66.92, 29.26, 24.31, 21.32.

LCMS C₃H₂N₃O₂ requires [M−THP]⁻, 112.02. Observed: [M−THP]⁻, 122.2.

### 1-(Tetrahydro-2H-pyran-2-yl)-1H-pyrazol-3-amine

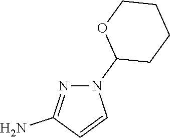 [SMILES: Nc1ccn(C2CCCCO2)n1]

General procedure 4 was followed using 3-nitro-1-(tetrahydro-2H-pyran-2-yl)-1H-pyrazole (197.2 mg, 1 mmol) and Pd/C (5.3 mg, 0.05 mmol) to afford the title compound which was used without further purification (167 mg, quant).

¹H NMR (500 MHz, DMSO-d₆) δ 7.43 (d, J=2.3 Hz, 1H), 5.46 (d, J=2.3 Hz, 1H), 5.04 (dd, J=10.2, 2.3 Hz, 1H), 4.59 (br s, 2H), 3.89-3.83 (m, 1H), 3.57-3.48 (m, 1H), 2.04-1.95 (m, 1H), 1.93-1.87 (m, 1H), 1.81-1.75 (m, 1H), 1.65-1.55 (m, 1H), 1.50-1.40 (m, 2H).

¹³C NMR (101 MHz, DMSO-d₆) δ 155.18, 129.39, 93.26, 86.09, 66.55, 29.59, 24.75, 22.36.

### 4-Chloro-N-(1-(tetrahydro-2H-pyran-2-yl)-1H-pyrazol-3-yl)pyridin-2-amine

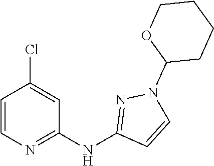 [SMILES: Clc1ccnc(Nc2ccn(C3CCCCO3)n2)c1]

General procedure 2 was followed using 2-bromo-4-chloropyridine (925 mg, 4.81 mmol), 1-(tetrahydro-2H-pyran-2-yl)-1H-pyrazol-3-amine (804 mg, 4.81 mmol), Pd(OAc)2 (54.1 mg, 0.241 mmol), Xantphos (278.3 mg, 0.481 mmol), and Cs₂CO3 (1.568 g, 4.81 mmol) in toluene at 90° C. for 18 hours. Flash column chromatography [petrol:ethyl acetate 0→100%] afforded the title compound (358 mg, 27%).

¹H NMR (500 MHz, DMSO-d₆) δ 9.59 (s, 1H), 8.09 (d, J=5.4 Hz, 1H), 7.73 (d, J=2.3 Hz, 1H), 7.41 (d, J=0.8 Hz, 1H), 6.80 (dd, J=5.4, 0.8 Hz, 1H), 6.33 (d, J=2.3 Hz, 1H), 5.28 (dd, J=10.5, 2.1 Hz, 1H), 3.92 (d, J=10.5 Hz, 1H), 3.67-3.56 (m, 1H), 2.13-2.02 (m, 1H), 1.98-1.87 (m, 2H), 1.70-1.62 (m, 1H), 1.55-1.48 (m, 2H).

¹³C NMR (101 MHz, DMSO-d₆) δ 156.02, 149.23, 149.01, 143.13, 129.31, 114.02, 108.43, 96.54, 86.35, 66.66, 29.62, 24.69, 22.10.

LCMS C₁₃H₁₅³⁹ClN₄O requires [M+H]⁺ 278.09, observed [M+H]⁺ 279.2.

### 7-(3,3-Dimethylbut-1-yn-1-yl)-5-(2-((1-(tetrahydro-2H-pyran-2-yl)-1H-pyrazol-3--yl)amino)pyridin-4-y)-1H-indazol-amine

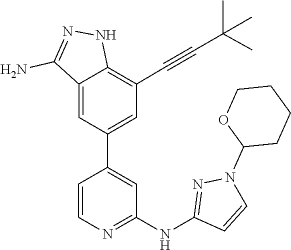 [SMILES: CC(C)(C)C#Cc1cc(-c2ccnc(Nc3ccn(C4CCCCO4)n3)c2)cc2c(N)n[nH]c12]

General procedure 1 was followed using 7-(3,3-dimethylbut-1-yn-1-yl)-5-(4,4,5,5-tetramethyl-1,3,2-dioxaborolan-2-yl)-1H-indazol-3-amine (118.7 mg, 0.35 mmol), 4-chloro-N-(1-(tetrahydro-2H-pyran-2-yl)-1H-pyrazol-3-yl)pyridin-2-amine (68.1 mg, 0.244 mmol), Pd(dtbpf)Cl₂ (22.8 mg, 0.035 mmol), and Cs2CO3 (228 mg, 0.7 mmol) in 9:1 dioxane:water at 120° C. for 6 hours under MW irradiation. Flash column chromatography [petrol:ethyl acetate 0→100%] using basic silica afforded the title compound (76.1 mg, 71%).

¹H NMR (400 MHz, DMSO-d₆) δ 11.80 (br s, 1H), 9.29 (br s, 1H), 8.15 (d, J=5.3 Hz, 1H), 8.12 (d, J=1.5 Hz, 1H), 7.71 (d, J=2.4 Hz, 1H), 7.65 (d, J=0.7 Hz, 1H), 7.51 (d, J=1.5 Hz, 1H), 7.03 (dd, J=5.3, 0.7 Hz, 1H), 6.40 (d, J=2.4 Hz, 1H), 5.59 (br s, 2H), 5.30 (dd, J=9.5, 2.5 Hz, 1H), 3.95-3.86 (m, 1H), 3.66-3.56 (m, 1H), 2.22-2.08 (m, 1H), 2.02-1.90 (m, 2H), 1.75-1.61 (m, 1H), 1.58-1.49 (m, 2H), 1.37 (s, 9H).

¹³C NMR (101 MHz, DMSO-d₆) δ 155.81, 150.53, 149.70, 148.52, 148.12, 141.37, 129.27, 127.87, 127.32, 119.04, 114.93, 111.84, 106.12, 105.50, 103.20, 96.47, 86.08, 74.43, 66.34, 30.69, 29.49, 27.90, 24.76, 22.00.

LCMS C₂₆H₂₉N₇O requires [M−THP+H]⁺ 372.19, observed [M−THP+H]⁺372.3.

### 5-(2-((1H-Pyrazol-3-yl)amino)pyridin-4-yl)-7-(3,3-dimethylbut-1-yn-1-yl)-1H-indazol-3-amine SU1759

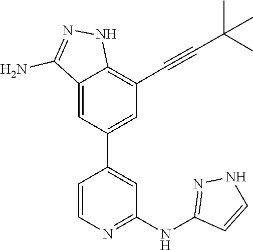 [SMILES: CC(C)(C)C#Cc1cc(-c2ccnc(Nc3cc[nH]n3)c2)cc2c(N)n[nH]c12]

To a round bottomed flask was added 7-(3,3-dimethylbut-1-yn-1-yl)-5-(2-((1-(tetrahydro-2H-pyran-2-yl)-1H-pyrazol-3-yl)amino)pyridin-4-yl)-1H-indazol-3-amine (66.4 mg, 0.148 mmol) and 1:1 TFA:DCM (5 mL) and the mixture was stirred at room temperature for 18 hours. The mixture was diluted with saturated aqueous NaHCO₃ solution, and the resultant precipitate was collected by vacuum filtration. Reverse phase flash column chromatography afforded the title compound (28.5 mg, 53%)

¹H NMR (400 MHz, DMSO-d₆) δ 12.12 (br s, 1H), 11.80 (br s, 1H), 9.20 (br s, 1H), 8.15 (d, J=5.3 Hz, 1H), 8.09 (d, J=1.5 Hz, 1H), 7.58 (br s, 1H), 7.56 (d, J=0.5 Hz, 1H), 7.49 (d, J=1.5 Hz, 1H), 7.00 (dd, J=5.3, 0.5 Hz, 1H), 6.30 (br s, 1H), 5.60 (br s, 2H), 1.37 (s, 9H).

¹³C NMR (101 MHz, DMSO-d₆) δ 156.00, 150.50, 148.66, 148.07, 141.34, 128.08, 127.45, 119.08, 114.91, 111.80, 105.99, 105.43, 103.30, 74.48, 30.70, 27.91. Three C not observed.

LCMS C₂₁H₂₁N₇ requires [M+H]⁺ 372.19, observed [M+H]⁺ 1 372.2.

### Example 311—5-(2-((1H-Pyrazol-4-yl)amino)pyridin-4-yl)-7-(3,3-dimethylbut-1-yn-1-yl)-1H-indazol-3-amine SU1760

### Route to SU1760

### 4-Nitro-1-(tetrahydro-2H-pyran-2-yl)-1H-pyrazole

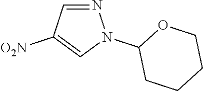 [SMILES: O=[N+]([O-])c1cnn(C2CCCCO2)c1]

General procedure 3 was followed using 4-nitro-1H-pyrazole (113.1 mg, 1 mmol), PTSA (17.2 mg, 0.1 mmol), and dihydropyran (183 μL, 2 mmol). Flash column chromatography [petrol:ethyl acetate 0→25%] afforded the title compound (197 mg, quant).

¹H NMR (500 MHz, DMSO-d₆) δ 9.02 (s, 1H), 8.30 (s, 1H), 5.50 (dd, J=9.7, 2.4 Hz, 1H), 3.98-3.91 (m, 1H), 3.69-3.61 (m, 1H), 2.15-2.05 (m, 1H), 1.98-1.88 (m, 2H), 1.73-1.61 (m, 1H), 1.59-1.50 (m, 2H).

¹³C NMR (101 MHz, DMSO-d₆) δ 135.37, 129.47, 87.47, 66.94, 29.15, 24.31, 21.38. One C not observed.

LCMS C₃H₂N₃O₂ requires [M−THP]⁻, 112.02. Observed: [M−THP]⁻, 122.2.

### 1-(Tetrahydro-2H-pyran-2-yl)-1H-pyrazol-4-amine

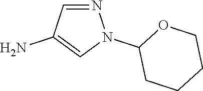 [SMILES: Nc1cnn(C2CCCCO2)c1]

General procedure 4 was followed using 4-nitro-1-(tetrahydro-2H-pyran-2-yl)-1H-pyrazole (197.2 mg, 1 mmol) and Pd/C (5.3 mg, 0.05 mmol) to afford the title compound which was used without further purification (167 mg, quant).

¹H NMR (500 MHz, DMSO-d₆) δ 7.10 (s, 1H), 6.96 (s, 1H), 5.16 (dd, J=9.9, 2.3 Hz, 1H), 3.85 (br s, 2H), 3.60-3.50 (m, 1H), 2.02-1.93 (m, 1H), 1.93-1.87 (m, 1H), 1.84-1.77 (m, 1H), 1.67-1.56 (m, 1H), 1.54-1.43 (m, 3H).

¹³C NMR (101 MHz, DMSO-d₆) δ 131.30, 129.97, 114.35, 86.71, 66.55, 29.72, 24.69, 22.18.

### 4-Chloro-N-(1-(tetrahydro-2H-pyran-2-yl)-1H-pyrazol-4-yl)pyridin-2-amine

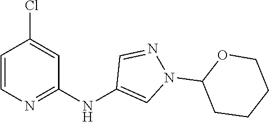 [SMILES: Clc1ccnc(Nc2cnn(C3CCCCO3)c2)c1]

General procedure 2 was followed using 2-bromo-4-chloropyridine (385 mg, 2 mmol), 1-(tetrahydro-2H-pyran-2-yl)-1H-pyrazol-4-amine (334 mg, 2 mmol), Pd(OAc)2 (9 mg, 0.04 mmol), Xantphos (34.7 mg, 0.06 mmol), and Cs2CO3 (658 mg, 2 mmol) in toluene at 60° C. for 18 hours. Flash column chromatography [petrol:ethyl acetate 0→50%] afforded the title compound (422 mg, 76%

¹H NMR (500 MHz, DMSO-d₆) δ 9.06 (s, 1H), 8.11-8.07 (m, 2H), 7.48 (s, 1H), 6.73-6.68 (m, 2H), 5.34 (dd, J=9.9, 2.1 Hz, 1H), 3.91 (d, J=12.1 Hz, 1H), 3.66-3.57 (m, 1H), 2.10-2.01 (m, 1H), 2.00-1.86 (m, 1H), 1.69-1.62 (m, 1H), 1.55-1.47 (m, 2H).

¹³C NMR (101 MHz, DMSO-d₆) δ 156.58, 149.27, 142.59, 130.74, 123.61, 118.47, 112.96, 108.07, 86.90, 66.71, 29.74, 24.63, 22.06.

LCMS C₁₃H₁₅³⁹ClN₄O requires [M+H]⁺ 278.09, observed [M+H]⁺ 279.2.

### 7-(3,3-Dimethylbut-1-yn-1-yl)-5-(2-((1-(tetrahydro-2H-pyran-2-yl)-1H-pyrazol-4-yl)amino)pyridin-4-yl)-1H-indazol-3-amine

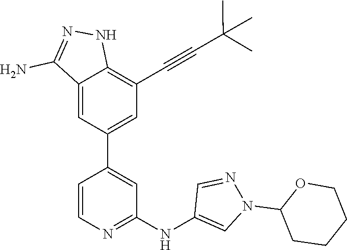 [SMILES: CC(C)(C)C#Cc1cc(-c2ccnc(Nc3cnn(C4CCCCO4)c3)c2)cc2c(N)n[nH]c12]

General procedure 1 was followed using 7-(3,3-dimethylbut-1-yn-1-yl)-5-(4,4,5,5-tetramethyl-1,3,2-dioxaborolan-2-yl)-1H-indazol-3-amine (118.7 mg, 0.35 mmol), 4-chloro-N-(1-(tetrahydro-2H-pyran-2-yl)-1H-pyrazol-4-yl)pyridin-2-amine (97.6 mg, 0.35 mmol), Pd(dtbpf)Cl2 (22.8 mg, 0.035 mmol), and Cs₂CO3 (228 mg, 0.7 mmol) in 9:1 dioxane:water at 120° C. for 6 hours under MW irradiation. Flash column chromatography [petrol:ethyl acetate 0→100%] using basic silica afforded the title compound (94.9 mg, 60%).

¹H NMR (500 MHz, DMSO-d₆) δ 11.80 (br s, 1H), 8.86 (br s, 1H), 8.15 (d, J=5.3 Hz, 1H), 8.13 (s, 1H), 8.10 (s, 1H), 7.50 (s, 2H), 6.97-6.91 (m, 2H), 5.59 (br s, 2H), 5.35 (d, J=10.4 Hz, 1H), 3.92 (d, J=11.5 Hz, 1H), 3.66-3.60 (m, 1H), 2.15-2.03 (m, 1H), 1.96-1.88 (m, 2H), 1.72-1.61 (m, 1H), 1.59-1.52 (m, 2H), 1.36 (s, 9H).

¹³C NMR not obtained.

LCMS C₂₆H₂₉N₇O requires [M+H]⁺ 445.24, observed [M−THP+H]⁺ 456.3.

### 5-(2-((1H-Pyrazol-4-yl)amino)pyridin-4-yl)-7-(3,3-dimethylbut-1-yn-1-yl)-1H-indazol-3-amine SU1760

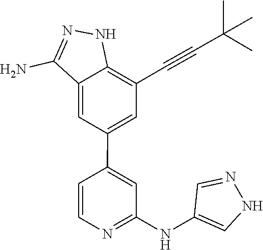 [SMILES: CC(C)(C)C#Cc1cc(-c2ccnc(Nc3cn[nH]c3)c2)cc2c(N)n[nH]c12]

To a round bottomed flask was added 7-(3,3-dimethylbut-1-yn-1-yl)-5-(2-((1-(tetrahydro-2H-pyran-2-yl)-1H-pyrazol-4-yl)amino)pyridin-4-yl)-1H-indazol-3-amine (85.2 mg, 0.187 mmol) and 1:1 TFA:DCM (5 mL) and the mixture was stirred at room temperature for 18 hours. The mixture was diluted with saturated aqueous NaHCO₃ solution, and the resultant precipitate was collected by vacuum filtration. Reverse phase flash column chromatography afforded the title compound (35 mg, 51%)

¹H NMR (400 MHz, DMSO-d₆) δ 12.44 (br s, 1H), 11.80 (br s, 1H), 8.72 (br s, 1H), 8.13 (d, J=6.0 Hz, 1H), 8.09 (d, J=1.5 Hz, 1H), 7.75 (br s, 2H), 7.49 (d, J=1.5 Hz, 1H), 6.94-6.90 (m, 2H), 5.60 (br s, 2H), 1.36 (s, 9H).

¹³C NMR (101 MHz, DMSO-d₆) δ 156.77, 150.48, 148.20, 147.98, 141.31, 127.81, 127.42, 123.52, 118.86, 114.90, 110.64, 105.46, 105.37, 103.21, 74.47, 30.70, 27.90.

### Example 312—7-(3,3-Dimethylbut-1-yn-1-yl)-5-(2-((5-methyl-1H-pyrazol-3-yl)amino)pyridin-4-yl)-1H-indazol-3-amine SU1745

### Route to SU1745

### 5-Methyl-3-nitro-1-(tetrahydro-2H-pyran-2-yl)-1H-pyrazole

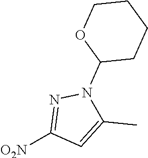

3,4-Dihydro-2H-pyran (1.3 g, 1.5 mL, 15.8 mmol, 2 Eq) was added to a solution of 5-methyl-3-nitro-1H-pyrazole (1 g, 7.9 mmol, 1 Eq) and p-toluenesulfonic acid monohydrate (150 mg, 0.8 mmol, 0.1 Eq) in anhydrous THF (5 mL). The reaction mixture was stirred under nitrogen atmosphere at 65° C. for 3 h. The organic solvent was evaporated under reduced pressure. The crude mixture was extracted between EtOAc (20 mL) and water (10 mL). The organic layer was washed with brine (2×10 mL), dried over anhydrous Na₂SO₄ and evaporated under reduced pressure. The crude residue was purified using column chromatography (50% EtOAc in petroleum ether) to give the titled product as white solid (1.2 g, 5.7 mmol, 72%).

¹H NMR (500 MHz, DMSO) δ 6.89 (s, 1H), 5.59 (dd, J=9.5, 2.7 Hz, 1H), 3.93-3.86 (m, 1H), 3.69 (dtd, J=8.9, 6.6, 4.6 Hz, 1H), 2.39 (s, 3H), 2.20 (dddd, J=13.3, 11.8, 9.4, 4.1 Hz, 1H), 2.01 (dtd, J=13.3, 4.2, 1.6 Hz, 1H), 1.93 (dt, J=13.2, 3.5 Hz, 1H), 1.76-1.63 (m, 1H), 1.56 (dq, J=10.2, 5.9, 4.8 Hz, 2H).

### 5-Methyl-1-(tetrahydro-2H-pyran-2-yl)-1H-pyrazol-3-amine

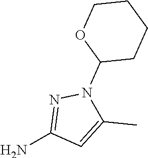

Pd/C 10% (0.1 g, 0.02 Eq) was added to a solution of 5-methyl-3-nitro-1-(tetrahydro-2H-pyran-2-yl)-1H-pyrazole (1 g, 4.7 mmol, 1 Eq) in MeOH (10 mL). The reaction mixture was allowed to stir under H₂ gas at rt for 4 h. The reaction mixture was filtered through a pad of Celite and evaporated under reduced pressure to give the titled product as green oil (0.8 g, 4.4 mmol, 94%).

¹H NMR (500 MHz, DMSO) δ 5.31 (s, 1H), 5.08 (dd, J=10.0, 2.4 Hz, 1H), 4.50 (s, 2H), 3.86 (ddd, J=11.0, 4.1, 2.1 Hz, 1H), 3.56 (td, J=10.9, 3.8 Hz, 1H), 2.27-2.14 (m, 1H), 2.14 (s, 3H), 2.00-1.90 (m, 1H), 1.75 (dq, J=13.3, 3.4 Hz, 1H), 1.62 (tdd, J=12.8, 10.4, 4.5 Hz, 1H), 1.48 (p, J=4.2 Hz, 2H).

### 4-Chloro-N-(5-methyl-1-(tetrahydro-2H-pyran-2-yl)-1H-pyrazol-3-yl)pyridin-2-amine

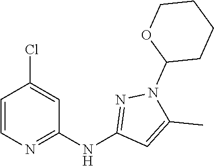 [SMILES: Cc1cc(Nc2cc(Cl)ccn2)nn1C1CCCCO1]

A mixture of palladium(II) acetate (62 mg, 0.28 mmol, 0.05 Eq) and xantphos (319 mg, 0.55 mmol, 0.1 Eq) in anhydrous toluene (2 mL) was stirred under nitrogen atmosphere for 15 min. The resulting mixture was added to a mixture of 2-bromo-4-chloropyridine (1.6 g, 8.3 mmol, 1.5 Eq), 5-methyl-1-(tetrahydro-2H-pyran-2-yl)-1H-pyrazol-3-amine (1 g, 5.5 mmol, 1 Eq) and Cs₂CO₃ (3.6 g, 11 mmol, 2 Eq) in toluene (4 mL) under nitrogen atmosphere. The reaction mixture was allowed to stir at 110° C. for 18 h. The reaction mixture was cooled and extracted between EtOAc (20 mL) and water (10 mL). The organic layer was washed with brine (2×10 mL), dried over anhydrous Na₂SO₄ and evaporated under reduced pressure. The crude residue was purified using column chromatography (50% EtOAc in petroleum ether) to give the titled product as white solid (0.5 g, 1.7 mmol, 31%).

¹H NMR (500 MHz, DMSO) δ 9.52 (s, 1H), 8.09 (d, J=5.4 Hz, 1H), 7.41 (d, J=1.9 Hz, 1H), 6.79 (dd, J=5.4, 1.9 Hz, 1H), 6.18 (s, 1H), 5.29 (dd, J=9.7, 2.5 Hz, 1H), 3.92-3.86 (m, 1H), 3.67-3.58 (m, 1H), 2.27 (s, 3H), 2.27-2.19 (m, 1H), 2.02 (dt, J=12.8, 4.2 Hz, 1H), 1.87 (dq, J=13.3, 3.4 Hz, 1H), 1.69 (tq, J=12.7, 4.4 Hz, 1H), 1.60-1.47 (m, 2H).

### 4-Chloro-N-(5-methyl-1H-pyrazol-3-yl)pyridin-2-amine

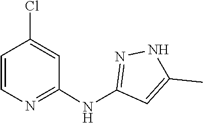

Trifluoroacetic acid (7.5 g, 5 mL, 65.8 mmol, 40 Eq) was added to a solution of 4-chloro-N-(5-methyl-1-(tetrahydro-2H-pyran-2-yl)-1H-pyrazol-3-yl)pyridin-2-amine (0.5 g, 1.7 mmol, 1 Eq) in DCM (5 mL) at 0° C. The reaction mixture was stirred at rt for 5 h. The reaction was quenched with saturated solution of NaHCO₃ (5 mL). The produced ppt was filtered, washed with water (5 mL) then DCM (2 mL) and dried to give the titled product as white solid (300 mg, 1.4 mmol, 85%).

¹H NMR (500 MHz, DMSO) δ 11.84 (s, 1H), 9.37 (s, 1H), 8.07 (d, J=5.5 Hz, 1H), 7.47 (s, 1H), 6.77 (dd, J=5.4, 2.0 Hz, 1H), 6.00 (s, 1H), 2.20 (s, 3H).

### 7-(3,3-Dimethylbut-1-yn-1-yl)-5-(2-((5-methyl-1H-pyrazol-3-yl)amino)pyridin-4-yl)-1H-indazol-3-amine SU1745

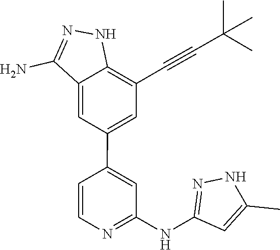 [SMILES: Cc1cc(Nc2cc(-c3cc(C#CC(C)(C)C)c4[nH]nc(N)c4c3)ccn2)n[nH]1]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), 4-chloro-N-(5-methyl-1H-pyrazol-3-yl)pyridin-2-amine (0.31 g, 1.47 mmol, 1 Eq), cesium carbonate (1 g, 2.94 mmol, 2 Eq) and 1,1′-bis(di-tert-butylphosphino) ferrocene palladium chloride (144 mg, 0.22 mmol, 0.15 Eq) in dioxane (4 mL) and water (1 mL) was stirred at 120° C. for 4 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (5% MeOH in EtOAc) followed by HPLC purification (50% MeCN in H₂O) to give the titled product as white solid (260 mg, 0.67 mmol, 46%).

¹H NMR (500 MHz, DMSO) δ 11.80 (s, 1H), 9.07 (s, 1H), 8.14 (d, J=5.3 Hz, 1H), 8.09 (d, J=1.7 Hz, 1H), 7.61 (s, 1H), 7.49 (d, J=1.7 Hz, 1H), 6.99 (d, J=5.5 Hz, 1H), 6.07 (s, 1H), 5.60 (s, 2H), 2.21 (s, 3H), 1.38 (s, 9H).

LCMS: For C₂₂H₂₃N₇ requires 385.48 found 386.3 (M+H).

### Example 313—7-(3,3-Dimethylbut-1-yn-1-yl)-5-(2-((3-methyl-1H-pyrazol-4-yl)amino)pyridin-4-yl)-1H-indazol-3-amine SU1750

### Route to SU1750

### 3-Methyl-4-nitro-1-(tetrahydro-2H-pyran-2-yl)-1H-pyrazole

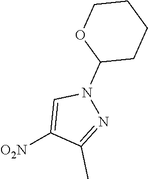

3,4-Dihydro-2H-pyran (2.0 g, 2.2 mL, 23.6 mmol, 2 Eq) was added to a solution of 3-methyl-4-nitro-1H-pyrazole (1.5 g, 11.8 mmol, 1 Eq) and p-toluenesulfonic acid monohydrate (225 mg, 1.18 mmol, 0.1 Eq) in anhydrous THF (10 mL). The reaction mixture was stirred under nitrogen atmosphere at 65° C. for 3 h. The organic solvent was concentrated under reduced pressure. The crude mixture was extracted between EtOAc (20 mL) and water (10 mL). The organic layer was washed with brine (2×10 mL), dried over anhydrous Na₂SO₄ and evaporated under reduced pressure. The crude residue was purified using column chromatography (50% EtOAc in petroleum ether) to give the titled product as yellow oil (2.4 g, 11.36 mmol, 96%).

¹H NMR (500 MHz, DMSO) δ 8.92 (s, 1H), 5.42 (dd, J=9.9, 2.3 Hz, 1H), 3.99-3.94 (m, 1H), 3.64 (ddd, J=11.5, 8.6, 6.7 Hz, 1H), 2.67 (s, 1H), 2.44 (s, 3H), 2.14-2.04 (m, 1H), 1.97-1.86 (m, 2H), 1.66 (dtd, J=15.3, 11.4, 11.0, 6.5 Hz, 1H), 1.55 (q, J=3.7 Hz, 2H).

### 3-Methyl-1-(tetrahydro-2H-pyran-2-yl)-1H-pyrazol-4-amine

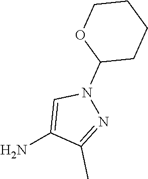

Pd/C 10% (0.24 g, 0.02 Eq) was added to a solution of 3-methyl-4-nitro-1-(tetrahydro-2H-pyran-2-yl)-1H-pyrazole (2.4 g, 11.36 mmol, 1 Eq) in MeOH (10 mL). The reaction mixture was allowed to stir under H₂ gas at rt for 4 h. The reaction mixture was filtered through a pad of Celite and evaporated under educed pressure to give the titled product as yellow solid (1.8 g, 9.9 mmol, 87%).

¹H NMR (500 MHz, DMSO) δ 7.05 (s, 1H), 5.08 (dd, J=10.1, 2.5 Hz, 1H), 3.90-3.81 (m, 1H), 3.66 (s, 2H), 3.63-3.50 (m, 1H), 2.12 (s, 1H), 2.01 (s, 3H), 2.00-1.95 (m, 1H), 1.95-1.88 (m, 1H), 1.79 (dt, J=12.7, 3.2 Hz, 1H), 1.62 (dq, J=11.4, 3.8 Hz, 1H), 1.49 (dd, J=7.5, 3.8 Hz, 1H).

### 4-Chloro-N-(3-methyl-1-(tetrahydro-2H-pyran-2-yl)-1H-pyrazol-4-yl)pyridin-2-amine

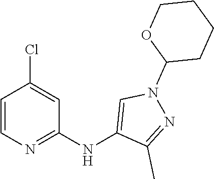 [SMILES: Cc1nn(C2CCCCO2)cc1Nc1cc(Cl)ccn1]

A mixture of palladium(II) acetate (97 mg, 0.43 mmol, 0.05 Eq) and xantphos (498 mg, 0.86 mmol, 0.1 Eq) in anhydrous toluene (3 mL) was stirred under nitrogen atmosphere for 15 min. The resulting mixture was added to a mixture of 2-bromo-4-chloropyridine (1.3 g, 6.88 mmol, 0.8 Eq), 3-methyl-1-(tetrahydro-2H-pyran-2-yl)-1H-pyrazol-4-amine (1.56 g, 8.6 mmol, 1 Eq) and Cs₂CO₃ (2.8 g, 8.6 mmol, 1 Eq) in toluene (6 mL) under nitrogen atmosphere. The reaction mixture was allowed to stir at 90° C. for 18 h. The reaction mixture was cooled and extracted between EtOAc (40 mL) and water (15 mL). The organic layer was washed with brine (2×10 mL), dried over anhydrous Na₂SO₄ and evaporated under reduced pressure. The crude residue was purified using column chromatography (60% EtOAc in petroleum ether) to give the titled product as yellow oil (400 mg, 1.4 mmol, 20%).

¹H NMR (500 MHz, DMSO) δ 8.39 (s, 1H), 8.06 (d, J=4.9 Hz, 2H), 6.75 (d, J=1.9 Hz, 1H), 6.70 (dd, J=5.5, 1.8 Hz, 1H), 5.25 (dd, J=10.2, 2.4 Hz, 1H), 3.98-3.85 (m, 1H), 3.60 (ddd, J=11.5, 8.2, 5.9 Hz, 1H), 2.13 (s, 3H), 2.06 (tdd, J=12.6, 10.1, 4.0 Hz, 1H), 1.94 (dtd, J=9.5, 3.9, 2.0 Hz, 1H), 1.87 (dt, J=12.8, 3.2 Hz, 1H), 1.65 (dtdd, J=16.2, 12.4, 9.2, 3.8 Hz, 1H), 1.58-1.48 (m, 2H).

### 7-(3,3-Dimethylbut-1-yn-1-yl)-5-(2-((3-methyl-1-(tetrahydro-2H-pyran-2-yl)-1H-pyrazol-4-yl)amino)pyridin-4-yl)-1H-indazol-3-amine

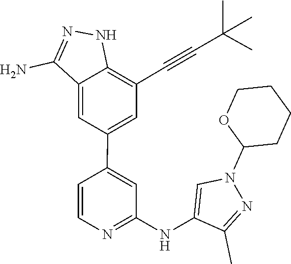 [SMILES: Cc1nn(C2CCCCO2)cc1Nc1cc(-c2cc(C#CC(C)(C)C)c3[nH]nc(N)c3c2)ccn1]

A solution of 7-(3,3-dimethylbut-1-yn-1-yl)-5-(4,4,5,5-tetramethyl-1,3,2-dioxaborolan-2-yl)-1H-indazol-3-amine (0.38 g, 1.1 mmol, 1.2 Eq), 4-chloro-N-(3-methyl-1-(tetrahydro-2H-pyran-2-yl)-1H-pyrazol-4-yl)pyridin-2-amine (0.27 g, 0.92 mmol, 1 Eq), cesium carbonate (0.6 g, 1.84 mmol, 2 Eq) and 1,1′-bis(di-tert-butylphosphino) ferrocene palladium chloride (150 mg, 0.23 mmol, 0.25 Eq) in dioxane (4 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) to give the titled product as yellow solid (300 mg, 64 mmol, 70%).

¹H NMR (500 MHz, DMSO) δ 11.82 (s, 1H), 8.21 (s, 1H), 8.18-8.10 (m, 3H), 7.52 (d, J=1.7 Hz, 1H), 7.11-7.07 (m, 1H), 6.96 (dd, J=5.4, 1.7 Hz, 1H), 5.60 (s, 2H), 5.27 (dd, J=10.2, 2.4 Hz, 1H), 3.92 (d, J=12.1 Hz, 1H), 3.67-3.56 (m, 1H), 2.23 (s, 1H), 2.19 (s, 3H), 2.13-2.02 (m, 1H), 1.98-1.86 (m, 1H), 1.72-1.60 (m, 1H), 1.53 (td, J=8.6, 7.2, 3.9 Hz, 2H), 1.37 (s, 9H).

### 7-(3,3-Dimethylbut-1-yn-1-yl)-5-(2-((3-methyl-1H-pyrazol-4-yl)amino)pyridin-4-yl)-1H-indazol-3-amine SU1750

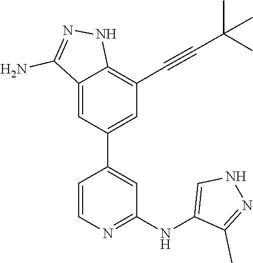 [SMILES: Cc1n[nH]cc1Nc1cc(-c2cc(C#CC(C)(C)C)c3[nH]nc(N)c3c2)ccn1]

Trifluoroacetic acid (2.9 g, 2 mL, 25.6 mmol, 40 Eq) was added to a solution of 7-(3,3-dimethylbut-1-yn-1-yl)-5-(2-((3-methyl-1-(tetrahydro-2H-pyran-2-yl)-1H-pyrazol-4-yl)amino)pyridin-4-yl)-1H-indazol-3-amine (0.3 g, 0.64 mmol, 1 Eq) in DCM (3 mL) at 0° C. The reaction mixture was stirred at rt for 5 h. The reaction was quenched with saturated solution of NaHCO₃ (5 mL). The reaction mixture was extracted between EtOAc (20 mL) and water (5 mL). The organic layer was washed with brine (2×5 mL), dried over anhydrous Na₂SO₄ and evaporated under reduced pressure. The crude residue was purified using reversed-phase column chromatography (50% MeCN in H₂O) to give the titled product as yellow solid (100 mg, 0.26 mmol, 40%).

¹H NMR (500 MHz, DMSO) δ 11.79 (s, 1H), 8.08 (d, J=5.5 Hz, 2H), 8.01 (s, 1H), 7.48 (s, 1H), 6.90 (d, J=5.4 Hz, 2H), 5.60 (s, 2H), 2.17 (s, 3H), 1.37 (s, 9H). LC-MS: For C₂₂H₂₃N₇ requires 385.48 found 386.4 (M+H).

### Example 314—N-(4-(3-Amino-7-(3,3-dimethylbut-1-yn-1-yl)-1H-indazol-5-yl)pyridin-2-yl)thiazol-2-amine SU1762

### Route to SU1762

### N-(4-Chloropyridin-2-yl)thiazol-2-amine

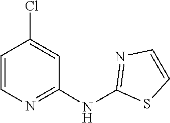 [SMILES: Clc1ccnc(Nc2nccs2)c1]

A mixture of palladium(II) acetate (56 mg, 0.25 mmol, 0.05 Eq) and xantphos (289 mg, 0.5 mmol, 0.1 Eq) in anhydrous toluene (1 mL) was stirred under nitrogen atmosphere for 15 min. The resulting mixture was added to a mixture of 2-bromo-4-chloropyridine (1.4 g, 7.5 mmol, 1.5 Eq), thiazol-2-amine (0.5 g, 4.99 mmol, 1 Eq) and Cs₂CO₃ (3 g, 10 mmol, 2 Eq) in toluene (2 mL) under nitrogen atmosphere. The reaction mixture was allowed to stir at 110° C. for 18 h. The organic solvent was removed under reduced pressure. The crude residue was stirred in MeOH (10 mL) for 30 min., filtered and washed with MeOH (2×3 mL). The filtrate was concentrated under reduced pressure. The crude residue was purified by column chromatography (30% EtOAc in petroleum ether) to give the tilted product as yellow solid (315 mg, 1.49 mmol, 30%).

¹H NMR (500 MHz, DMSO) δ 11.40 (s, 1H), 8.29 (d, J=5.5 Hz, 1H), 7.42 (d, J=3.6 Hz, 1H), 7.16 (d, J=1.8 Hz, 1H), 7.07 (d, J=3.6 Hz, 1H), 7.02 (dd, J=5.5, 1.8 Hz, 1H).

LCMS: For C₃H₆ClN₃S requires 211.67 found 212.3 (M+H).

### N-(4-(3-Amino-7-(3,3-dimethylbut-1-yn-1-yl)-1H-indazol-5-yl)pyridin-2-yl)thiazol-2-amine SU1762

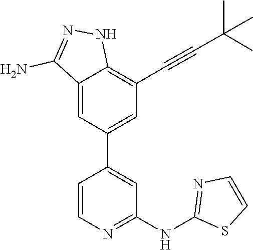 [SMILES: CC(C)(C)C#Cc1cc(-c2ccnc(Nc3nccs3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (309 mg, 0.9 mmol, 1.2 Eq), N-(4-chloropyridin-2-yl)thiazol-2-amine (160 mg, 0.76 mmol, 1 Eq), cesium carbonate (0.5 g, 1.52 mmol, 2 Eq) and 1,1′-bis(di-tert-butylphosphino) ferrocene palladium chloride (124 mg, 0.19 mmol, 0.15 Eq) in dioxane (3 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) followed by HPLC purification (50% MeCN in water) to give the titled product as white solid (23 mg, 0.06 mmol, 8%).

¹H NMR (500 MHz, DMSO) δ 11.87 (s, 1H), 11.19 (s, 1H), 8.33 (d, J=5.4 Hz, 1H), 8.15 (d, J=1.7 Hz, 1H), 7.54 (d, J=1.6 Hz, 1H), 7.40 (d, J=3.6 Hz, 1H), 7.34 (d, J=1.7 Hz, 1H), 7.23 (dd, J=5.4, 1.6 Hz, 1H), 7.02 (d, J=3.6 Hz, 1H), 5.64 (s, 2H), 1.38 (s, 9H).

LCMS: For C₂₁H₂₀N₆S requires 388.49 found 389.2 (M+H).

### Example 315—N-(4-(3-Amino-7-(3,3-dimethylbut-1-yn-1-yl)-1H-indazol-5-yl)pyridin-2-yl)-4-methyloxazol-2-amine SU1746

### Route to SU1746

### N-(4-Chloropyridin-2-yl)-4-methyloxazol-2-amine

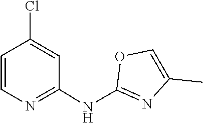

General procedure 2 was followed using 2-bromo-4-chloropyridine (192.4 mg, 1 mmol), 4-methyloxazol-2-amine (98.1 mg, 1 mmol), Pd(OAc)₂ (11.3 mg, 0.05 mmol), Xantphos (57.9 mg, 0.1 mmol), and Cs₂CO3 (651.6 mg, 2 mmol) in toluene at 110° C. for 18 hours. Flash column chromatography [petrol:ethyl acetate 0→30%] afforded the title compound (63.6 mg, 30%).

¹H NMR (400 MHz, DMSO-d₆) δ 10.95 (br s, 1H), 8.22 (d, J=5.4 Hz, 1H), 8.17 (s, 1H), 7.42 (d, J=1.3 Hz, 1H), 7.07 (dd, J=5.4, 1.3 Hz, 1H), 2.07 (d, J=0.9 Hz, 3H). ¹³C NMR (101 MHz, DMSO-d₆) δ 154.87, 153.44, 149.48, 144.05, 135.08, 128.74, 116.92, 109.47, 11.72.

LCMS C₉H₈³⁵ClN₃O requires [M+H]⁺, 209.04. Observed: [M+H]⁺, 210.2.

### N-(4-(3-Amino-7-(3,3-dimethylbut-1-yn-1-yl)-1H-indazol-5-yl)pyridin-2-yl)-4-methyloxazol-2-amine SU1746

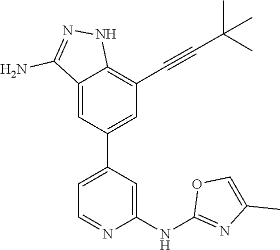 [SMILES: Cc1coc(Nc2cc(-c3cc(C#CC(C)(C)C)c4[nH]nc(N)c4c3)ccn2)n1]

General procedure 1 was followed using 7-(3,3-dimethylbut-1-yn-1-yl)-5-(4,4,5,5-tetramethyl-1,3,2-dioxaborolan-2-yl)-1H-indazol-3-amine (118.7 mg, 0.35 mmol), N-(4-chloropyridin-2-yl)-4-methyloxazol-2-amine (73.4 mg, 0.35 mmol), Pd(dtbpf)Cl₂ (22.8 mg, 0.035 mmol), and Cs2CO3 (228.1 mg, 0.7 mmol) in 9:1 dioxane:water at 120° C. for 2.5 hours. Flash column chromatography [petrol:ethyl acetate:methanol 0→100%→20%] afforded the title compound (55.8 mg, 41%).

¹H NMR (500 MHz, DMSO-d₆) δ 11.84 (br s, 1H), 10.59 (br s, 1H), 8.32-8.22 (m, 2H), 8.14 (s, 1H), 7.55 (s, 1H), 7.42 (s, 1H), 7.24 (d, J=4.7 Hz, 1H), 5.62 (br s, 2H), 2.08 (s, 3H), 1.37 (s, 9H).

¹³C NMR (101 MHz, DMSO-d₆) δ 155.56, 153.14, 150.55, 149.50, 148.37, 141.35, 135.04, 128.47, 127.73, 127.58, 119.32, 114.94, 114.70, 107.01, 105.57, 103.36, 74.46, 30.67, 27.90, 11.77.

LCMS C₂₂H₂₂N₆O requires [M+H]⁺, 386.19. Observed: [M+H]⁺, 387.3.

### Example 316—N-(4-(3-Amino-7-(3,3-dimethylbut-1-yn-1-yl)-1H-indazol-5-yl)pyridin-2-yl)-4-(trifluoromethyl)oxazol-2-amine SU1753

### Route to SU1753

### N-(4-Chloropyridin-2-yl)-4-(trifluoromethyl)oxazol-2-amine

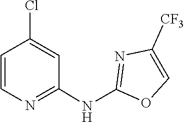 [SMILES: FC(F)(F)c1coc(Nc2cc(Cl)ccn2)n1]

A mixture of palladium(II) acetate (37 mg, 0.17 mmol, 0.05 Eq) and xantphos (191 mg, 0.33 mmol, 0.1 Eq) in anhydrous toluene (1 mL) was stirred under nitrogen atmosphere for 15 min. The resulting mixture was added to a mixture of 2-bromo-4-chloropyridine (940 mg, 4.9 mmol, 1.5 Eq), 4-(trifluoromethyl)oxazol-2-amine (0.5 g, 3.3 mmol, 1 Eq) and Cs₂CO₃ (2 g, 6.6 mmol, 2 Eq) in toluene (4 mL) under nitrogen atmosphere. The reaction mixture was allowed to stir at 110° C. for 18 h. The reaction mixture was cooled and extracted with EtOAc (20 mL) and water (30 mL). The organic layer was washed with brine (2×20 mL), dried over anhydrous Na₂SO₄ and evaporated under reduced pressure. The crude residue was purified by column chromatography (40% EtOAc in petroleum ether) to give the tilted product as yellow solid (300 mg, 1.1 mmol, 34%).

¹H NMR (500 MHz, DMSO) δ 11.55 (s, 1H), 8.51 (d, J=1.9 Hz, 1H), 8.28 (d, J=5.4 Hz, 1H), 7.99 (d, J=1.8 Hz, 1H), 7.16 (dd, J=5.4, 1.8 Hz, 1H). ¹³C NMR (126 MHz, DMSO) δ 162.92, 157.08, 153.15, 149.98, 144.77,134.84, 130.01, 129.70, 122.35, 118.32, 110.59.

### N-(4-(3-Amino-7-(3,3-dimethylbut-1-yn-1-yl)-1H-indazol-5-yl)pyridin-2-yl)-4-(trifluoromethyl)oxazol-2-amine SU1753

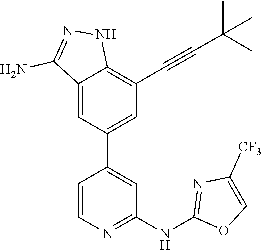 [SMILES: CC(C)(C)C#Cc1cc(-c2ccnc(Nc3nc(C(F)(F)F)co3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (309 mg, 0.9 mmol, 1.2 Eq), N-(4-chloropyridin-2-yl)-4-(trifluoromethyl)oxazol-2-amine (200 mg, 0.76 mmol, 1 Eq), cesium carbonate (0.5 g, 1.52 mmol, 2 Eq) and 1,1′-bis(di-tert-butylphosphino) ferrocene palladium chloride (124 mg, 0.19 mmol, 0.15 Eq) in dioxane (4 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (70% EtOAc in petroleum ether) followed by HPLC purification (50% MeCN in water) to give the titled product as yellow solid (150 mg, 0.34 mmol, 45%).

¹H NMR (500 MHz, DMSO) δ 11.89 (s, 1H), 11.22 (s, 1H), 8.51 (t, J=1.9 Hz, 1H), 8.33 (d, J=5.3 Hz, 1H), 8.20 (d, J=1.6 Hz, 1H), 8.17 (s, 1H), 7.59 (d, J=1.7 Hz, 1H), 7.35 (dd, J=5.3, 1.7 Hz, 1H), 5.63 (s, 2H), 1.37 (s, 9H).

### Example 317—N-(4-(3-amino-7-(3,3-dimethylbut-1-yn-1-yl)-1H-indazol-5-yl)pyridin-2-yl)-3,5-dimethylisoxazol-4-amine SU1764

### Route to SU1764

### N-(4-Chloropyridin-2-yl)-3,5-dimethylisoxazol-4-amine

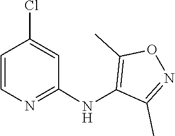

A mixture of palladium(II) acetate (20 mg, 0.09 mmol, 0.05 Eq) and xantphos (154.50 mg, 0.27 mmol, 0.15 Eq) in anhydrous toluene (1 mL) was stirred under argon atmosphere for 15 min. The resulting mixture was added to a mixture of 2-bromo-4-chloropyridine (513.81 mg, 2.7 mmol, 1.5 Eq), 3,5-dimethylisoxazol-4-amine (200 mg, 1.78 mmol, 1 Eq) and Cs₂CO3 (1,160 g, 3.56 mmol, 2 Eq) in toluene (4 mL) under argon atmosphere. The reaction mixture was allowed to stir at 60° C. for 18 h.

The reaction was then diluted with EtOAc (20 mL) and the organic layer was washed with water (2×10 mL), dried over anhydrous Na₂SO₄ and evaporated under reduced pressure. The crude residue was purified by column chromatography (50% EtOAc in petroleum ether) to give the tilted product as a solid (259 mg, 1.16 mmol, 65%).

¹H NMR (500 MHz, DMSO-d₆) δ 8.27 (s, 1H), 7.97 (d, J=5.5 Hz, 1H), 6.74 (dd, J=5.5, 1.8 Hz, 1H), 6.58 (d, J=1.1 Hz, 1H), 2.24 (s, 3H), 2.04 (s, 3H) ppm. ¹³C NMR (126 MHz, DMSO-d₆) 5 62.8, 158.5, 158.3, 149.4, 143.3, 115.9, 113.7, 107.1, 10.6, 9.6 ppm.

### N-(4-(3-amino-7-(3,3-dimethylbut-1-yn-1-yl)-1H-indazol-5-yl)pyridin-2-yl)-3,5-dimethylisoxazol-4-amine SU1764

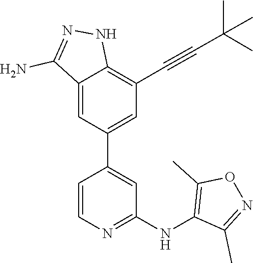 [SMILES: Cc1noc(C)c1Nc1cc(-c2cc(C#CC(C)(C)C)c3[nH]nc(N)c3c2)ccn1]

A solution of 7-(3,3-dimethylbut-1-yn-1-yl)-5-(4,4,5,5-tetramethyl-1,3,2-dioxaborolan-2-yl)-1H-indazol-3-amine (183.20 mg, 0.54 mmol, 1.2 Eq), N-(4-chloropyridin-2-yl)-3,5-dimethylisoxazol-4-amine (100 mg, 0.45 mmol, 1 Eq), cesium carbonate (293.24 mg, 0.9 mmol, 2 Eq) and 1,1′-bis(di-tert-butylphosphino) ferrocene palladium chloride (45.62 mg, 0.07 mmol, 0.15 Eq) in dioxane (3 mL) and water (1 mL) was heated in the microwave at 120° C. for 3 h. The reaction was then diluted with EtOAc (20 mL) and the organic layer was washed with water (2×10 mL) and brine (1×5 mL), dried over anhydrous Na₂SO₄ and evaporated under reduced pressure.

The crude residue was purified using column chromatography (40% to 90% EtOAc in petroleum ether) followed by HPLC purification to give the titled product as a solid (131.56 mg, 0.33 mmol, 73%).

¹H NMR (500 MHz, DMSO-d₆) δ 11.79 (s, 1H), 8.07 (s, 1H), 8.04-7.99 (m, 2H), 7.49 (d, J=1.2 Hz, 1H), 6.81 (s, 1H), 6.60 (d, J=4.5 Hz, 1H), 5.60 (s, 2H), 2.28 (s, 3H), 2.08 (s, 3H), 1.36 (s, 9H) ppm.

LCMS (ESI+ve): Calculated for C₂₃H₂₄N₆O requires 400.49 found 401.3 (M+H).

### Example 318—5-(2-((1H-Imidazol-4-yl)amino)pyridin-4-yl)-7-(3,3-dimethylbut-1-yn-1-yl)-1H-indazol-3-amine SU1769

### Route to SU1769

### 4-Nitro-1-(tetrahydro-2H-pyran-2-yl)-1H-imidazole

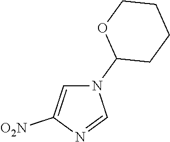 [SMILES: O=[N+]([O-])c1cn(C2CCCCO2)cn1]

3,4-Dihydro-2H-pyran (1.85 g, 2.0 mL, 22.1 mmol, 2.5 Eq) was added to a solution of 4-nitro-1H-imidazole (1 g, 8.85 mmol, 1 Eq) and p-toluenesulfonic acid monohydrate (150 mg, 0.9 mmol, 0.1 Eq) in anhydrous THF (10 mL) and DMSO (1 mL). The reaction mixture was stirred under nitrogen atmosphere at 65° C. for 3 h. The organic solvent was concentrated under reduced pressure. The crude mixture was extracted between EtOAc (20 mL) and water (10 mL). The organic layer was washed with brine (2×10 mL), dried over anhydrous Na₂SO₄ and evaporated under reduced pressure. The crude residue was purified using column chromatography (90% EtOAc in petroleum ether) to give the titled product as yellow oil (1.7 g, 8.85 mmol, 100%).

¹H NMR (500 MHz, DMSO) δ 8.51 (d, J=1.6 Hz, 1H), 8.04 (d, J=1.6 Hz, 1H), 5.50-5.44 (m, 1H), 3.97 (dtd, J=11.7, 3.7, 2.0 Hz, 1H), 3.64 (ddd, J=11.6, 7.5, 5.2 Hz, 1H), 1.98 (ddd, J=10.8, 8.7, 4.1 Hz, 2H), 1.89 (dtd, J=13.4, 3.9, 1.9 Hz, 1H), 1.70-1.58 (m, 1H), 1.54 (ddt, J=9.1, 6.0, 3.9 Hz, 2H).

### 1-(Tetrahydro-2H-pyran-2-yl)-1H-imidazol-4-amine

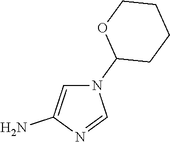 [SMILES: Nc1cn(C2CCCCO2)cn1]

Pd/C 10% (0.2 g, 0.02 Eq) was added to a solution of 4-nitro-1-(tetrahydro-2H-pyran-2-yl)-1H-imidazole (1.7 g, 8.85 mmol, 1 Eq) in MeOH (10 mL). The reaction mixture was allowed to stir under H₂ gas at rt for 6 h. The reaction mixture was filtered through a pad of Celite and evaporated under educed pressure to give the titled product as brown oil (1.3 g, 7.78 mmol, 88%).

¹H NMR (500 MHz, DMSO) δ 7.32 (d, J=1.6 Hz, 1H), 6.24 (d, J=1.6 Hz, 1H), 5.11 (dd, J=7.4, 5.1 Hz, 1H), 3.91 (dp, J=11.4, 2.4 Hz, 1H), 3.56 (ddd, J=14.1, 7.5, 5.1 Hz, 1H), 1.88-1.76 (m, 3H), 1.69-1.54 (m, 1H), 1.50 (tp, J=8.3, 4.8, 4.1 Hz, 2H).

### 4-Chloro-N-(1-(tetrahydro-2H-pyran-2-yl)-1H-imidazol-4-yl)pyridin-2-amine

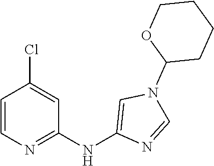 [SMILES: Clc1ccnc(Nc2cn(C3CCCCO3)cn2)c1]

A mixture of palladium(II) acetate (47 mg, 0.21 mmol, 0.05 Eq) and xantphos (243 mg, 0.42 mmol, 0.1 Eq) in anhydrous toluene (1 mL) was stirred under nitrogen atmosphere for 15 min. The resulting mixture was added to a mixture of 2-bromo-4-chloropyridine (0.8 g, 4.2 mmol, 1 Eq), 1-(tetrahydro-2H-pyran-2-yl)-1H-imidazol-4-amine (0.7 g, 4.2 mmol, 1 Eq) and Cs₂CO₃ (2 g, 6.3 mmol, 1.5 Eq) in toluene (4 mL) under nitrogen atmosphere. The reaction mixture was allowed to stir at 80° C. for 18 h. The reaction mixture was cooled and extracted between EtOAc (20 mL) and water (10 mL). The organic layer was washed with brine (2×10 mL), dried over anhydrous Na₂SO₄ and evaporated under reduced pressure. The crude residue was purified using column chromatography (80% EtOAc in petroleum ether) to give the titled product as yellow solid (0.4 g, 1.4 mmol, 34%).

¹H NMR (500 MHz, DMSO) δ 9.46 (s, 1H), 8.12 (d, J=5.5 Hz, 1H), 7.62 (d, J=1.6 Hz, 1H), 7.38 (d, J=1.6 Hz, 1H), 6.97 (d, J=1.9 Hz, 1H), 6.72 (dd, J=5.5, 1.9 Hz, 1H), 5.33-5.24 (m, 1H), 3.96 (ddd, J=12.2, 4.4, 2.4 Hz, 1H), 3.68-3.56 (m, 1H), 1.92 (ddt, J=11.5, 7.7, 3.8 Hz, 3H), 1.65 (tt, J=11.7, 4.0 Hz, 1H), 1.55 (ddt, J=12.0, 8.1, 3.6 Hz, 2H).

### 7-(3,3-Dimethylbut-1-yn-1-yl)-5-(2-((1-(tetrahydro-2H-pyran-2-yl)-1H-imidazol-4-yl)amino)pyridin-4-yl)-1H-indazol-3-amine

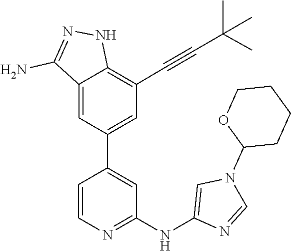 [SMILES: CC(C)(C)C#Cc1cc(-c2ccnc(Nc3cn(C4CCCCO4)cn3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (0.44 g, 1.3 mmol, 1.2 Eq), 4-chloro-N-(1-(tetrahydro-2H-pyran-2-yl)-1H-imidazol-4-yl)pyridin-2-amine (0.3 g, 1.1 mmol, 1 Eq), cesium carbonate (0.7 g, 2.2 mmol, 2 Eq) and 1,1′-bis(di-tert-butylphosphino) ferrocene palladium chloride (179 mg, 0.28 mmol, 0.25 Eq) in dioxane (4 mL) and water (1 mL) was heated in the microwave at 120° C. for 3 h. The reaction mixture was poured onto ice-cold water (15 mL) and filtered, washed with water (2×5 mL) and dried. The crude solid was purified using column chromatography (90% EtOAc in petroleum ether) followed by HPLC (60% MeCN in water) to give the titled product as white solid (100 mg, 0.2 mmol, 20%).

¹H NMR (500 MHz, DMSO) δ 11.80 (s, 1H), 9.16 (s, 1H), 8.18 (d, J=5.4 Hz, 1H), 8.08 (d, J=1.7 Hz, 1H), 7.60 (d, J=1.5 Hz, 1H), 7.49 (d, J=1.6 Hz, 1H), 7.45 (d, J=1.7 Hz, 1H), 7.17 (d, J=1.7 Hz, 1H), 6.95 (dd, J=5.5, 1.7 Hz, 1H), 5.60 (s, 2H), 5.29 (dd, J=7.1, 5.1 Hz, 1H), 4.01-3.93 (m, 1H), 3.68-3.59 (m, 1H), 1.98-1.88 (m, 3H), 1.66 (td, J=8.3, 3.8 Hz, 1H), 1.62-1.50 (m, 2H), 1.38 (s, 9H).

LC-MS: For C₂₆H₂₉N₇O requires 455.57 found 456.3 (M+H).

### 5-(2-((1H-Imidazol-4-yl)amino)pyridin-4-yl)-7-(3,3-dimethylbut-1-yn-1-yl)-1H-indazol-3-amine SU1769

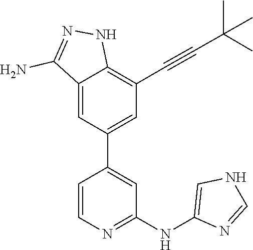 [SMILES: CC(C)(C)C#Cc1cc(-c2ccnc(Nc3c[nH]cn3)c2)cc2c(N)n[nH]c12]

Hydrochloric acid (2N, 2.2 mL, 4.4 mmol, 10 Eq) was added to a solution of 7-(3,3-dimethylbut-1-yn-1-yl)-5-(2-((1-(tetrahydro-2H-pyran-2-yl)-1H-imidazol-4-yl)amino)pyridin-4-yl)-1H-indazol-3-amine (SU1768, 0.1 g, 0.22 mmol, 1 Eq) in EtOH (10 mL). The reaction mixture was allowed to stir under reflux for 2 h. The reaction mixture was cooled, quenched with saturated solution of sodium carbonate solution (10 mL) and extracted between EtOAc (30 mL) and water (10 mL). The organic layer was washed with brine (3×5 mL), dried over anhydrous sodium sulfate and evaporated under reduced pressure. The crude residue was purified through HPLC (60% MeCN in water) to give the titled product as yellow solid (57 mg, 0.15 mmol, 70%).

¹H NMR (500 MHz, DMSO) δ 11.79 (s, 1H), 11.68 (s, 1H), 9.04 (s, 1H), 8.15 (d, J=5.3 Hz, 1H), 8.07 (d, J=1.6 Hz, 1H), 7.48 (d, J=1.7 Hz, 1H), 7.43 (d, J=1.5 Hz, 1H), 7.26 (d, J=1.5 Hz, 1H), 7.18 (d, J=1.7 Hz, 1H), 6.92 (dd, J=5.5, 1.7 Hz, 1H), 5.59 (s, 2H), 1.38 (s, 9H).

LCMS: For C₂₁H₂₁N₇ requires 371.45 found 372.3 (M+H).

### Example 319—5-(2-((4H-1,2,4-Triazol-3-yl)amino)pyridin-4-yl)-7-(3,3-dimethylbut-1-yn-1-yl)-1H-indazol-3-amine SU1751

### Route to SU1751

### 3-Nitro-4-(tetrahydro-2H-pyran-2-yl)-4H-1,2,4-triazole

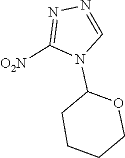 [SMILES: O=[N+]([O-])c1nncn1C1CCCCO1]

3,4-Dihydro-2H-pyran (2.2 g, 2.5 mL, 26.3 mmol, 2 Eq) was added to a solution of 3-nitro-4H-1,2,4-triazole (1.5 g, 13.15 mmol, 1 Eq) and p-toluenesulfonic acid monohydrate (250 mg, 1.32 mmol, 0.1 Eq) in anhydrous THF (10 mL). The reaction mixture was stirred under nitrogen atmosphere at 65° C. for 3 h. The organic solvent was concentrated under reduced pressure. The crude mixture was extracted between EtOAc (20 mL) and water (10 mL). The organic layer was washed with brine (2×10 mL), dried over anhydrous Na₂SO₄ and evaporated under reduced pressure. The crude residue was purified using column chromatography (60% EtOAc in petroleum ether) to give the titled product as yellow oil (2.6 g, 13.12 mmol, 100%).

¹H NMR (500 MHz, DMSO) δ 9.08 (s, 1H), 5.75 (dd, J=8.1, 3.9 Hz, 1H), 3.95 (dt, J=11.0, 3.8 Hz, 1H), 3.71 (ddd, J=11.4, 8.5, 4.7 Hz, 1H), 2.08 (ddd, J=10.1, 8.6, 4.3 Hz, 2H), 1.94 (dt, J=13.6, 4.6 Hz, 1H), 1.70 (ddq, J=19.4, 9.9, 5.1 Hz, 1H), 1.64-1.51 (m, 2H).

### 4-(Tetrahydro-2H-pyran-2-yl)-4H-1,2,4-triazol-3-amine

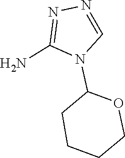 [SMILES: Nc1nncn1C1CCCCO1]

Pd/C 10% (0.27 g, 0.02 Eq) was added to a solution of 3-nitro-4-(tetrahydro-2H-pyran-2-yl)-4H-1,2,4-triazole (2.5 g, 12.6 mmol, 1 Eq) in MeOH (10 mL). The reaction mixture was allowed to stir under H₂ gas at rt for 4 h. The reaction mixture was filtered through a pad of Celite and evaporated under educed pressure to give the titled product as yellow solid (2 g, 11.9 mmol, 94%).

¹H NMR (500 MHz, DMSO) δ 8.13 (s, 1H), 5.31 (s, 2H), 5.24 (dd, J=9.9, 2.6 Hz, 1H), 3.90 (dd, J=11.9, 3.7 Hz, 1H), 3.57 (ddd, J=11.3, 8.9, 4.7 Hz, 1H), 2.00 (dtd, J=12.9, 9.8, 9.2, 4.9 Hz, 1H), 1.88 (ddt, J=28.2, 12.9, 3.8 Hz, 2H), 1.60 (td, J=10.4, 8.2, 5.3 Hz, 1H), 1.51 (dd, J=8.3, 4.1 Hz, 2H).

### 4-Chloro-N-(4-(tetrahydro-2H-pyran-2-yl)-4H-1,2,4-triazol-3-yl)pyridin-2-amine

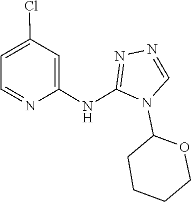 [SMILES: Clc1ccnc(Nc2nncn2C2CCCCO2)c1]

A mixture of palladium(II) acetate (67 mg, 0.3 mmol, 0.05 Eq) and xantphos (344 mg, 0.6 mmol, 0.1 Eq) in anhydrous toluene (2 mL) was stirred under nitrogen atmosphere for 15 min. The resulting mixture was added to a mixture of 2-bromo-4-chloropyridine (1.7 g, 8.9 mmol, 1.5 Eq), 4-(tetrahydro-2H-pyran-2-yl)-4H-1,2,4-triazol-3-amine (1 g, 6.0 mmol, 1 Eq) and Cs₂CO₃ (3.9 g, 11.9 mmol, 2 Eq) in toluene (5 mL) under nitrogen atmosphere. The reaction mixture was allowed to stir at 110° C. for 18 h. The reaction mixture was cooled and extracted between EtOAc (20 mL) and water (10 mL). The organic layer was washed with brine (2×10 mL), dried over anhydrous Na₂SO₄ and evaporated under reduced pressure. The crude residue was purified using column chromatography (40% EtOAc in petroleum ether) to give the titled product as white solid (0.65 g, 2.3 mmol, 39%).

¹H NMR (500 MHz, DMSO) δ 10.10 (s, 1H), 8.57 (s, 1H), 8.18 (d, J=5.4 Hz, 1H), 7.96 (d, J=1.9 Hz, 1H), 6.95 (dd, J=5.4, 1.9 Hz, 1H), 5.49 (dd, J=9.8, 2.3 Hz, 1H), 3.99-3.91 (m, 1H), 3.66 (ddd, J=13.7, 7.1, 5.0 Hz, 1H), 2.14-2.03 (m, 1H), 1.96 (dp, J=11.3, 3.9 Hz, 2H), 1.68 (dtd, J=12.7, 9.1, 8.7, 5.8 Hz, 1H), 1.55 (dq, J=9.7, 5.4, 4.5 Hz, 2H).

### 7-(3,3-Dimethylbut-1-yn-1-yl)-5-(2-((4-(tetrahydro-2H-pyran-2-yl)-4H-1,2,4-triazol-3-yl)amino)pyridin-4-yl)-1H-indazol-3-amine

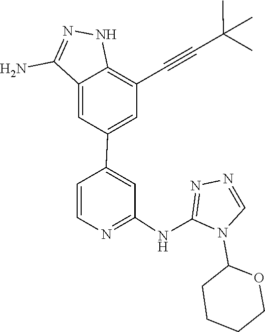 [SMILES: CC(C)(C)C#Cc1cc(-c2ccnc(Nc3nncn3C3CCCCO3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (0.15 g, 0.43 mmol, 1.2 Eq), 4-chloro-N-(4-(tetrahydro-2H-pyran-2-yl)-4H-1,2,4-triazol-3-yl)pyridin-2-amine (0.1 g, 0.36 mmol, 1 Eq), cesium carbonate (0.24 g, 0.72 mmol, 2 Eq) and 1,1′-bis(di-tert-butylphosphino) ferrocene palladium chloride (59 mg, 0.09 mmol, 0.25 Eq) in dioxane (4 mL) and water (1 mL) was heated in the microwave at 120° C. for 3 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was used in the next step without further purification (0.3 g).

### 5-(2-((4H-1,2,4-Triazol-3-yl)amino)pyridin-4-yl)-7-(3,3-dimethylbut-1-yn-1-yl)-1H-indazol-3-amine SU1751

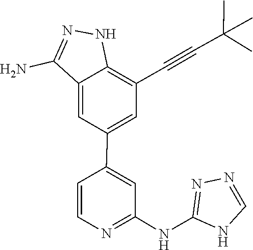 [SMILES: CC(C)(C)C#Cc1cc(-c2ccnc(Nc3nnc[nH]3)c2)cc2c(N)n[nH]c12]

Trifluoroacetic acid (3 g, 2 mL, 26.3 mmol, 40 Eq) was added to a solution of 7-(3,3-dimethylbut-1-yn-1-yl)-5-(2-((4-(tetrahydro-2H-pyran-2-yl)-4H-1,2,4-triazol-3-yl)amino)pyridin-4-yl)-1H-indazol-3-amine (0.3 g, 0.66 mmol, 1 Eq) in DCM (3 mL) at 0° C. The reaction mixture was stirred at rt for 5 h. The reaction was quenched with saturated solution of NaHCO₃ (5 mL). The reaction mixture was extracted between EtOAc (20 mL) and water (5 mL). The organic layer was washed with brine (2×5 mL), dried over anhydrous Na₂SO₄ and evaporated under reduced pressure. The crude residue was purified using reversed-phase column chromatography (40% MeCN in H₂O) followed by HPLC purification (40% MeCN in H₂O) to give the titled product as yellow solid (5 mg, 0.02 mmol, 3%).

¹H NMR (500 MHz, DMSO) δ 13.14 (s, 1H), 11.85 (s, 1H), 10.59 (s, 1H), 8.27 (s, 1H), 8.13 (d, J=1.6 Hz, 1H), 7.68 (s, 1H), 7.52 (d, J=1.6 Hz, 1H), 7.26 (d, J=39.9 Hz, 2H), 5.63 (s, 2H), 1.38 (s, 9H).

LCMS: For C₂₀H₂₀N₆ requires 372.44 found 372.9 (M+H).

### Example 320—7-(3,3-Dimethylbut-1-yn-1-yl)-5-(2-((2-methyl-2H-tetrazol-5-yl)amino)pyridin-4-yl)-1H-indazol-3-amine SU1766

### Route to SU1766

### 4-Chloro-N-(2-methyl-2H-tetrazol-5-yl)pyridin-2-amine

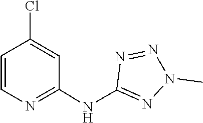

A mixture of palladium(II) acetate (113 mg, 0.5 mmol, 0.05 Eq) and xantphos (584 mg, 1.0 mmol, 0.1 Eq) in anhydrous toluene (1 mL) was stirred under nitrogen atmosphere for 15 min. The resulting mixture was added to a mixture of 2-bromo-4-chloropyridine (2.3 g, 12.12 mmol, 1.5 Eq), 2-methyl-2H-tetrazol-5-amine (1 g, 10.1 mmol, 1 Eq) and Cs₂CO₃ (5 g, 15.2 mmol, 1.5 Eq) in toluene (4 mL) under nitrogen atmosphere. The reaction mixture was allowed to stir at 80° C. for 18 h. The resulted precipitate was filtered, washed with water (3×10 mL) and petroleum ether (3×10 mL) and dried under vacuum to give the tilted product as brown solid and used in the next step without further purification.

### 7-(3,3-Dimethylbut-1-yn-1-yl)-5-(2-((2-methyl-2H-tetrazol-5-yl)amino)pyridin-4-yl)-1H-indazol-3-amine SU1766

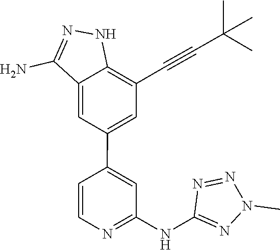 [SMILES: Cn1nnc(Nc2cc(-c3cc(C#CC(C)(C)C)c4[nH]nc(N)c4c3)ccn2)n1]

A solution of 7-(3,3-dimethylbut-1-yn-1-yl)-5-(4,4,5,5-tetramethyl-1,3,2-dioxaborolan-2-yl)-1H-indazol-3-amine (770 mg, 2.3 mmol, 1.2 Eq), 4-chloro-N-(2-methyl-2H-tetrazol-5-yl)pyridin-2-amine (400 mg, 1.9 mmol, 1 Eq), cesium carbonate (1 g, 3.8 mmol, 2 Eq) and 1,1′-bis(di-tert-butylphosphino) ferrocene palladium chloride (300 mg, 0.5 mmol, 0.15 Eq) in dioxane (3 mL) and water (1 mL) was heated in the microwave at 120° C. for 3 h. The reaction mixture was cooled, filtered through celite and extracted between EtOAc (10 mL) and water (5 mL). The organic layer was washed with brine (2×5 mL), dried over anhydrous Na₂SO₄ and evaporated under reduced pressure. The crude residue was purified using column chromatography (90% EtOAc in petroleum ether) followed trituration using MeOH (5×3 mL) to give the titled product as white solid (220 mg, 0.57 mmol, 30%).

¹H NMR (500 MHz, DMSO) δ 11.84 (s, 1H), 10.28 (s, 1H), 8.27 (d, J=5.3 Hz, 1H), 8.14 (d, J=1.6 Hz, 1H), 7.93-7.89 (m, 1H), 7.55 (d, J=1.6 Hz, 1H), 7.23 (dd, J=5.3, 1.6 Hz, 1H), 5.63 (s, 2H), 4.32 (s, 3H), 1.38 (s, 9H). ¹³C NMR (126 MHz, DMSO) δ 163.10, 154.67, 151.05, 150.00, 148.97, 141.88, 128.13, 128.03, 119.76, 115.44, 114.73, 106.91, 106.06, 103.91, 74.90, 31.18, 28.41.

LCMS: For C₂₀H₂₁N₉ requires 387.45 found 388.3 (M+H).

### Example 321—7-(3,3-Dimethylbut-1-yn-1-yl)-5-(2-(pyrimidin-2-ylamino)pyridin-4-yl)-1H-indazol-3-amine SU1767

### Route to SU1767

### N-(4-Chloropyridin-2-yl)pyrimidin-2-amine

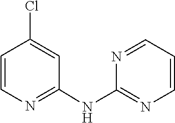 [SMILES: Clc1ccnc(Nc2ncccn2)c1]

A mixture of palladium(II) acetate (118 mg, 0.5 mmol, 0.05 Eq) and xantphos (608 mg, 1.1 mmol, 0.1 Eq) in anhydrous toluene (1 mL) was stirred under nitrogen atmosphere for 15 min. The resulting mixture was added to a mixture of 2-bromo-4-chloropyridine (2.4 g, 12.6 mmol, 1.5 Eq), pyrimidin-2-amine (1 g, 10.5 mmol, 1 Eq) and Cs₂CO₃ (5 g, 15.8 mmol, 1.5 Eq) in toluene (4 mL) under nitrogen atmosphere. The reaction mixture was allowed to stir at 80° C. for 18 h. The reaction mixture was cooled and extracted between EtOAc (20 mL) and water (10 mL). The organic layer was washed with brine (3×10 mL) dried over anhydrous sodium sulfate and evaporated under reduced pressure. The crude residue was purified by column chromatography (30% EtOAc in petroleum ether) to give the tilted product as yellow solid (0.9 g, 4.2 mmol, 40%).

### 7-(3,3-Dimethylbut-1-yn-1-yl)-5-(2-(pyrimidin-2-ylamino)pyridin-4-yl)-1H-indazol-3-amine SU1767

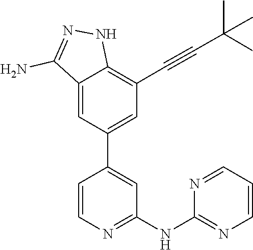 [SMILES: CC(C)(C)C#Cc1cc(-c2ccnc(Nc3ncccn3)c2)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (770 mg, 2.3 mmol, 1.2 Eq), N-(4-chloropyridin-2-yl)pyrimidin-2-amine (400 mg, 1.9 mmol, 1 Eq), cesium carbonate (1 g, 3.8 mmol, 2 Eq) and 1,1′-bis(di-tert-butylphosphino) ferrocene palladium chloride (300 mg, 0.5 mmol, 0.15 Eq) in dioxane (3 mL) and water (1 mL) was heated in the microwave at 120° C. for 3 h. The reaction mixture was cooled, poured onto ice-cold water (10 mL), filtered, washed with water (2×5 mL) and dried. The crude ppt was purified by column chromatography (90% EtOAc in petroleum ether) followed by HPLC purification (60% MeCN in water) to give the titled product as white solid (290 mg, 0.76 mmol, 40%).

¹H NMR (500 MHz, DMSO) δ 11.84 (s, 1H), 9.92 (s, 1H), 8.62 (d, J=4.8 Hz, 2H), 8.55 (d, J=1.7 Hz, 1H), 8.34 (d, J=5.2 Hz, 1H), 8.17 (d, J=1.7 Hz, 1H), 7.58 (d, J=1.6 Hz, 1H), 7.31 (dd, J=5.2, 1.7 Hz, 1H), 6.99 (t, J=4.8 Hz, 1H), 5.65 (s, 2H), 1.38 (s, 9H).

LCMS: For C₂₂H₂₁N₇ requires 383.46 found 384.3 (M+H).

### Example 322—7-(3,3-Dimethylbut-1-yn-1-yl)-5-(2-((tetrahydrofuran-3-yl)amino)pyridin-4-yl)-1H-indazol-3-amine SU1763

### Route to SU1763

### 4-Iodo-N-(tetrahydrofuran-3-yl)pyridin-2-amine

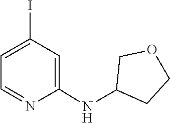 [SMILES: Ic1ccnc(NC2CCOC2)c1]

In a sealed 10 mL microwave vial, 2-fluoro-4-iodopyridine (200 mg, 0.89 mmol, 1 Eq) was dissolved in 3 mL of DMSO. Then tetrahydrofuran-3-amine (93.22 mg, 0.092 mL, 1.07 mmol, 1.2 Eq) and triethylamine (134.58 mg, 0.185 mL, 1.33 mmol, 1.5 Eq) were added and the resulting reaction mixture was heated at 100° C. under microwave irradiation for 4 hours.

The reaction was then diluted with EtOAc (10 mL) and the organic layer was washed with brine (2×5 mL), dried over anhydrous Na₂SO₄ and evaporated under reduced pressure. The crude residue was purified by column chromatography (20% EtOAc in petroleum ether) to give the titled product (194 mg, 0.67 mmol, 75%).

¹H NMR (500 MHz, DMSO-d₆) δ 7.68 (d, J=5.3 Hz, 1H), 6.92 (s, 1H), 6.89 (d, J=6.1 Hz, 1H), 6.83 (d, J=5.1 Hz, 1H), 4.31 (dd, J=9.8, 6.6 Hz, 1H), 3.81 (dt, J=14.7, 6.8 Hz, 2H), 3.70 (dt, J=13.9, 7.0 Hz, 1H), 3.48 (dd, J=8.8, 3.7 Hz, 1H), 2.14 (td, J=15.0, 7.5 Hz, 1H), 1.74 (dt, J=12.3, 5.5 Hz, 1H) ppm. ¹³C NMR (126 MHz, DMSO-d₆) δ 158.8, 148.3, 120.0, 116.9, 105.7, 72.8, 66.3, 51.1, 32.4 ppm.

LCMS (ESI+ve): Calculated for C₉H₁₁₁N20 requires 290.10 found 291.1 (M+H).

### 7-(3,3-Dimethylbut-1-yn-1-yl)-5-(2-((tetrahydrofuran-3-yl)amino)pyridin-4-yl)-1H-indazol-3-amine SU1763

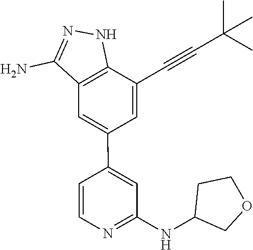 [SMILES: CC(C)(C)C#Cc1cc(-c2ccnc(NC3CCOC3)c2)cc2c(N)n[nH]c12]

To a solution of 7-(3,3-dimethylbut-1-yn-1-yl)-5-(4,4,5,5-tetramethyl-1,3,2-dioxaborolan-2-yl)-1H-indazol-3-amine (142.48 mg, 0.42 mmol, 1.1 Eq) in a degassed 9:1 solution of dioxane/water (8 mL), were added 4-iodo-N-(tetrahydrofuran-3-yl)pyridin-2-amine (110 mg, 0.38 mmol, 1 Eq), cesium carbonate (371.43 mg, 1.14 mmol, 3 Eq) and bis(triphenylphosphine)palladium chloride (26.67 mg, 0.038 mmol, 0.1 Eq) under argon atmosphere. The resulting reaction mixture was allowed to stir at 70° C. overnight.

The cooled reaction was diluted with EtOAc (20 mL) and washed with water (10 mL) and brine (2×10 mL). The organic layer was concentrated under reduced pressure and the crude was purified by flash column chromatography (0% to 100% EtOAc in petroleum ether) followed by HPLC purification to afford the titled compound as a solid (103 mg, 0.27 mmol, 72%).

¹H NMR (500 MHz, DMSO-d₆) δ 11.77 (s, 1H), 8.06 (s, 1H), 8.01 (d, J=5.3 Hz, 1H), 7.47 (s, 1H), 6.81 (d, J=5.3 Hz, 1H), 6.76 (s, 1H), 6.70 (d, J=6.2 Hz, 1H), 5.57 (s, 2H), 4.42 (d, J=5.1 Hz, 1H), 3.91-3.82 (m, 2H), 3.73 (dd, J=13.9, 7.9 Hz, 1H), 3.53 (dd, J=8.7, 3.8 Hz, 1H), 2.18 (dd, J=12.6, 7.3 Hz, 1H), 1.81 (dt, J=11.5, 5.8 Hz, 1H), 1.36 (s, 9H) ppm.

LCMS (ESI+ve): Calculated for C₂₂H₂₅N₅O requires 375.48 found 376.3 (M+H).

### Example 323—Methyl (4-(3-Amino-7-(4-hydroxyphenyl)-1H-indazol-5-yl)pyridin-2-yl)carbamate SU1744

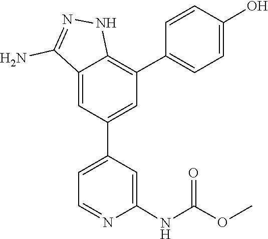 [SMILES: COC(=O)Nc1cc(-c2cc(-c3ccc(O)cc3)c3[nH]nc(N)c3c2)ccn1]

A solution of methyl (4-(3-amino-7-bromo-1H-indazol-5-yl)pyridin-2-yl)carbamate (1.1 g, 2.96 mmol, 1 Eq), (4-hydroxyphenyl)boronic acid (0.61 g, 4.44 mmol, 1.5 Eq), cesium carbonate (2.9 g, 8.88 mmol, 3 Eq) and [1,1′-bis(diphenylphosphino)ferrocene]dichloropalladium(II) (322 mg, 0.44 mmol, 0.15 Eq) in dioxane (8 mL) and water (2 mL) was stirred under nitrogen atmosphere at 80° C. for 1 h. The reaction mixture was poured onto cold water (10 mL). The produced ppt was filtered, washed with water (5 mL), dried, purified using column chromatography (20% MeOH in EtOAc) and followed by trituration with MeOH (×1) and Et₂O (×2) to give the titled product as white solid (0.4 g, 1.1 mmol, 37%).

¹H NMR (500 MHz, DMSO) δ 11.63 (s, 1H), 10.20 (s, 1H), 9.63 (s, 1H), 8.31 (d, J=5.2 Hz, 1H), 8.21 (s, 1H), 8.11 (s, 1H), 7.58 (d, J=8.2 Hz, 2H), 7.54 (d, J=1.5 Hz, 1H), 7.43 (dd, J=5.3, 1.6 Hz, 1H), 6.93 (d, J=8.2 Hz, 2H), 5.58 (s, 2H), 3.72 (s, 3H).

LCMS: For C₂₀H₁₇N₅O₃ requires 375.39 found 376.3 (M+H).

### Example 324—4-(3-Amino-5-(2-(oxetan-3-ylamino)pyridin-4-yl)-1H-indazol-7-yl)phenol SU1749

### Route to SU1749

### 7-Bromo-5-(2-(oxetan-3-ylamino)pyridin-4-yl)-1H-indazol-3-amine

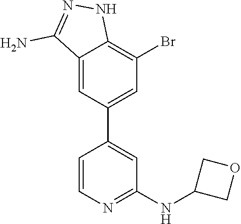 [SMILES: Nc1n[nH]c2c(Br)cc(-c3ccnc(NC4COC4)c3)cc12]

General procedure 1 was followed using 7-bromo-5-(4,4,5,5-tetramethyl-1,3,2-dioxaborolan-2-yl)-1H-indazol-3-amine (67.6 mg, 0.2 mmol), 4-iodo-N-(oxetan-3-yl)pyridin-2-amine (55.2 mg, 0.2 mmol), Pd₂Cl₂(PPh₃)₂(14 mg, 0.02 mmol), and Cs₂CO₃ (195.2 mg, 0.6 mmol) in 9:1 dioxane:water at 90° C. for 18 hours. Trituration from DCM afforded the title compound (29.0 mg, 40%).

¹H NMR (400 MHz, DMSO-d₆) δ 11.94 (br s, 1H), 8.12 (d, J=1.0 Hz, 1H), 8.01 (d, J=5.4 Hz, 1H), 7.74 (d, J=1.0 Hz, 1H), 7.26 (br d, J=6.2 Hz, 1H), 6.86 (d, J=5.4 Hz, 1H), 6.74 (s, 1H), 5.66 (br s, 2H), 5.00-4.90 (m, 1H), 4.83 (app. t, J=6.4 Hz, 2H), 4.47 (app. t, J=6.4 Hz, 2H).

¹³C NMR (101 MHz, DMSO-d₆) δ 158.36, 150.70, 148.24, 147.62, 140.07, 129.22, 127.03, 118.28, 116.04, 110.55, 104.91, 102.67, 77.95, 46.02.

LCMS C₁₅H₁₄⁷⁹BrN₅O requires [M+H]⁺, 359.04. Observed: [M+H]⁺, 360.1.

### 4-(3-Amino-5-(2-(oxetan-3-ylamino)pyridin-4-yl)-1H-indazol-7-yl)phenol SU1749

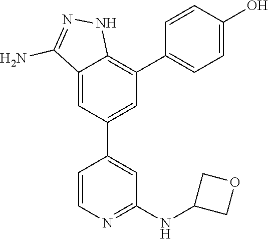 [SMILES: Nc1n[nH]c2c(-c3ccc(O)cc3)cc(-c3ccnc(NC4COC4)c3)cc12]

General procedure 1 was followed using 7-bromo-5-(2-(oxetan-3-ylamino)pyridin-4-yl)-1H-indazol-3-amine (102 mg, 0.283 mmol), (4-hydroxyphenyl)boronic acid (78.1 mg, 0.566 mmol), Pd₂(dppf)Cl₂ (20.7 mg, 0.028 mmol), and Cs₂CO3 (276.8 mg, 0.849 mmol) in 9:1 dioxane:water at 90° C. for 1 hour. Flash column chromatography [petrol:ethyl acetate:methanol 0→100%→50%] followed by reverse phase flash column chromatography [water:acetonitrile 0→50%] afforded the title compound (32.0 mg, 30%).

¹H NMR (400 MHz, DMSO-d₆) δ 11.65 (br s, 1H), 8.06 (s, 1H), 7.65 (d, J=6.2 Hz, 1H), 7.55 (d, J=8.5 Hz, 2H), 7.46 (s, 1H), 6.90 (d, J=8.5 Hz, 2H), 6.76 (br s, 1H), 6.46 (d, J=6.2 Hz, 1H), 5.55 (br s, 2H), 4.26-4.12 (m, 1H), 4.06-3.95 (m, 1H), 3.56-3.50 (m, 1H), 3.44-3.35 (m, 1H).

¹³C NMR (101 MHz, DMSO-d₆) δ 157.62, 156.96, 150.35, 150.25, 139.42, 135.64, 129.12, 127.66, 127.22, 124.12, 123.07, 117.36, 115.84, 115.52, 106.57, 104.04, 64.01, 51.63.

LCMS C₂₁H₁₉N₅O₂ requires [M+H]⁺, 373.15. Observed: [M+H]⁺, 374.3.

### Example 325—Methyl (4-(3-amino-7-(4-aminophenyl)-1H-indazol-5-yl)pyridin-2-yl)carbamate SU1755

### Route to SU1755

### Methyl (4-(3-amino-7-bromo-1H-indazol-5-yl)pyridin-2-yl)carbamate

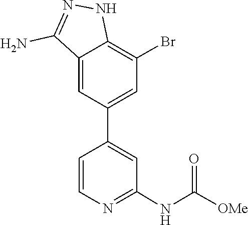 [SMILES: COC(=O)Nc1cc(-c2cc(Br)c3[nH]nc(N)c3c2)ccn1]

General procedure 1 was followed using 7-bromo-5-(4,4,5,5-tetramethyl-1,3,2-dioxaborolan-2-yl)-1H-indazol-3-amine (507 mg, 1.5 mmol), methyl (4-iodopyridin-2-yl)carbamate (417 mg, 1.5 mmol), Pd₂Cl₂(PPh₃)₂(105 mg, 0.15 mmol), and Cs₂CO3 (1.466 g, 4.5 mmol) in 9:1 dioxane:water at 70° C. for 18 hours. Flash column chromatography [DCM; MeOH 0→100%] on basic silica afforded the title compound (202 mg, 37%).

¹H NMR (500 MHz, DMSO-d₆) δ 12.00 (br s, 1H), 10.23 (br s, 1H), 8.30 (d, J=5.3 Hz, 1H), 8.20 (d, J=1.3 Hz, 1H), 8.14 (d, J=1.1 Hz, 1H), 7.79 (d, J=1.3 Hz, 1H), 7.36 (dd, J=5.3, 1.1 Hz, 1H), 5.71 (br s, 2H), 3.71 (s, 3H). ¹³C NMR (101 MHz, DMSO-d₆) δ Compound previously characterised.

LCMS C₁₄H₁₂₇₉BrN₅O₂ requires [M+H]⁺, 361.02. Observed: [M+H]⁺, 362.0.

### Methyl (4-(3-amino-7-(4-aminophenyl)-1H-indazol-5-yl)pyridin-2-yl)carbamate SU1755

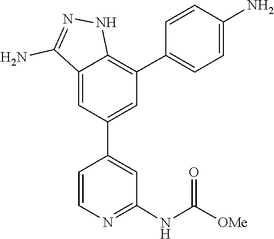 [SMILES: COC(=O)Nc1cc(-c2cc(-c3ccc(N)cc3)c3[nH]nc(N)c3c2)ccn1]

General procedure 1 was followed using methyl (4-(3-amino-7-bromo-1H-indazol-5-yl)pyridin-2-yl)carbamate (74.7 mg, 0.206 mmol), 4-(4,4,5,5-tetramethyl-1,3,2-dioxaborolan-2-yl)aniline (67.7 mg, 0.309 mmol), Pd₂(dppf)Cl₂ (14.6 mg, 0.02 mmol), and Cs₂CO3 (195.5 mg, 0.6 mmol) in 9:1 dioxane:water at 90° C. for 16 hours. Flash column chromatography [petrol:ethyl acetate:methanol 0→100%--*100%] using basic silica followed by reverse phase flash column chromatography [water:acetonitrile 0→50%] afforded the title compound (48.4 mg, 62%).

¹H NMR (500 MHz, DMSO-d₆) δ 11.55 (br s, 1H), 10.17 (br s, 1H), 8.29 (d, J=5.2 Hz, 1H), 8.19 (s, 1H), 8.03 (s, 1H), 7.49 (s, 1H), 7.44-7.39 (m, 3H), 6.71 (d, J=8.4 Hz, 2H), 5.52 (br s, 2H), 5.30 (br s, 2H), 3.70 (s, 3H).

¹³C NMR (101 MHz, DMSO-d₆) δ 154.19, 152.88, 150.29, 148.59, 148.23, 139.32, 128.58, 128.05, 124.94, 124.52, 122.83, 116.72, 116.38, 115.68, 114.20, 109.08, 51.82. One C not observed.

LCMS C₂₀H₁₈N₆O₂ requires [M+H]⁺, 374.15. Observed: [M+H]⁺, 375.1.

### Example 326

### Methyl (4-(7-(4-acetamidophenyl)-3-amino-1H-indazol-5-yl)pyridin-2-yl)carbamate SU1756

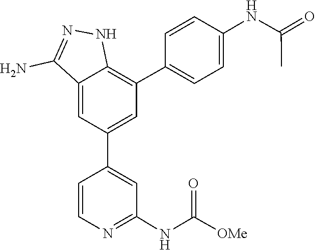 [SMILES: COC(=O)Nc1cc(-c2cc(-c3ccc(NC(C)=O)cc3)c3[nH]nc(N)c3c2)ccn1]

General procedure 1 was followed using methyl (4-(3-amino-7-bromo-1H-indazol-5-yl)pyridin-2-yl)carbamate (72.4 mg, 0.2 mmol), N-(4-(4,4,5,5-tetramethyl-1,3,2-dioxaborolan-2-yl)phenyl)acetamide (78.3 mg, 0.3 mmol), Pd₂(dppf)Cl₂ (14.6 mg, 0.02 mmol), and Cs2CO3 (195.5 mg, 0.6 mmol) in 9:1 dioxane:water at 90° C. for 16 hours. Reverse phase flash column chromatography [water:methanol 5→30%] followed by trituration from methanol afforded the title compound (13.0 mg, 16%).

¹H NMR (500 MHz, DMSO-d₆) δ 11.69 (br s, 1H), 10.19 (br s, 1H), 10.08 (br s, 1H), 8.30 (d, J=4.8 Hz, 1H), 8.20 (s, 1H), 8.14 (s, 1H), 7.74 (d, J=8.0 Hz, 2H), 7.68 (d, J=8.0 Hz, 2H), 7.59 (s, 1H), 7.43 (d, J=4.8 Hz, 1H), 5.60 (br s, 2H), 3.70 (s, 3H), 2.09 (s, 3H).

¹³C NMR (101 MHz, DMSO-d₆) δ 168.42, 154.21, 152.92, 150.44, 150.04, 148.29, 139.19, 138.98, 131.88, 128.31, 128.07, 123.95, 123.76, 119.34, 118.03, 116.44, 109.13, 51.84, 24.07.

One C not observed.

LCMS C₂₂H₂₀N₆O₃ requires [M+H]⁺, 416.16. Observed: [M+H]⁺, 417.2.

### Example 327—

### Methyl (4-(3-amino-7-(4-carbamoylphenyl)-1H-indazol-5-yl)pyridin-2-yl)carbamate SU1752

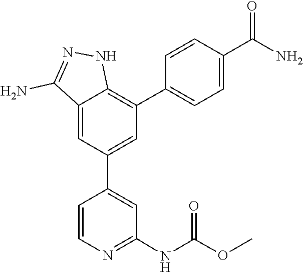 [SMILES: COC(=O)Nc1cc(-c2cc(-c3ccc(C(N)=O)cc3)c3[nH]nc(N)c3c2)ccn1]

A solution of methyl (4-(3-amino-7-bromo-1H-indazol-5-yl)pyridin-2-yl)carbamate (0.15 g, 0.4 mmol, 1 Eq), 4-(4,4,5,5-tetramethyl-1,3,2-dioxaborolan-2-yl)benzamide (0.15 g, 0.6 mmol, 1.5 Eq), cesium carbonate (0.4 g, 1.2 mmol, 3 Eq) and [1,1′-bis(diphenylphosphino)ferrocene]dichloropalladium(II) (44 mg, 0.06 mmol, 0.15 Eq) in dioxane (2 mL) and water (0.5 mL) was stirred under nitrogen atmosphere at 80° C. for 18 h. The reaction mixture was cooled and extracted between EtOAc (10 mL) and water (2 mL). The organic layer was dried over anhydrous sodium sulphate and evaporated. The crude residue was purified using reversed-phase column chromatography (50% MeCN in H₂O) followed by HPLC purification (50% MeCN in H₂O) to give the titled product as yellow solid (2 mg, 0.02 mmol, 5%).

¹H NMR (500 MHz, DMSO) δ 10.31 (s, 1H), 8.36 (d, J=5.2 Hz, 1H), 8.17 (d, J=1.6 Hz, 1H), 8.13 (s, 1H), 8.04 (d, J=8.4 Hz, 2H), 7.81 (d, J=8.4 Hz, 2H), 7.46 (s, 1H), 7.42 (dd, J=5.3, 1.7 Hz, 1H), 3.71 (s, 3H).

### Example 328—Methyl (4-(3-amino-7-(4-(morpholinomethyl)phenyl)-1H-indazol-5-yl)pyridin-2-yl)carbamate SU1757

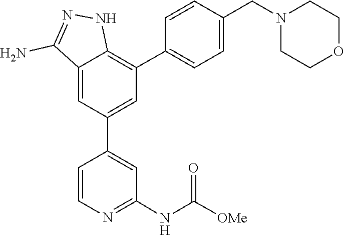 [SMILES: COC(=O)Nc1cc(-c2cc(-c3ccc(CN4CCOCC4)cc3)c3[nH]nc(N)c3c2)ccn1]

General procedure 1 was followed using methyl (4-(3-amino-7-bromo-1H-indazol-5-yl)pyridin-2-yl)carbamate (72.4 mg, 0.2 mmol), 4-(4-(4,4,5,5-tetramethyl-1,3,2-dioxaborolan-2-yl)benzyl)morpholine (90.9 mg, 0.3 mmol), Pd₂(dppf)Cl₂ (14.6 mg, 0.02 mmol), and Cs₂CO3 (195.5 mg, 0.6 mmol) in 9:1 dioxane:water at 90° C. for 16 hours. Flash column chromatography [petrol:ethyl acetate:methanol 0→100%--*100%] using basic silica afforded the title compound (47.0 mg, 34%).

¹H NMR (400 MHz, DMSO-d₆) δ 11.71 (br s, 1H), 10.20 (br s, 1H), 8.30 (d, J=5.2 Hz, 1H), 8.21 (d, J=1.3 Hz, 1H), 8.17 (d, J=1.3 Hz, 1H), 7.71 (d, J=8.2 Hz, 2H), 7.61 (d, J=1.7 Hz, 1H), 7.46 (d, J=8.2 Hz, 2H), 7.44 (dd, J=5.2, 1.7 Hz, 1H), 5.61 (br s, 2H), 3.70 (s, 3H), 3.65-3.58 (m, 4H), 2.46-2.38 (m, 4H).

¹³C NMR (101 MHz, DMSO-d₆) δ 154.19, 152.92, 150.40, 149.94, 148.28, 139.27, 137.43, 136.07, 129.52, 127.99, 127.82, 124.24, 123.86, 118.33, 116.37, 115.89, 109.07, 66.23, 62.17, 53.22, 51.82.

LCMS C₂₅H₂₆N₆O₃ requires [M+H]⁺, 458.21. Observed: [M+H]⁺, 459.2.

### Example 329—5-(2-Aminopyridin-4-yl)-7-(4-(2-morpholinoethyl)phenyl)-1H-indazol-3-amine SU1772

### Route to SU1772

### tert-Butyl (4-(3-amino-7-(4-(2-morpholinoethyl)phenyl)-1H-indazol-5-yl)pyridin-2-yl)carbamate

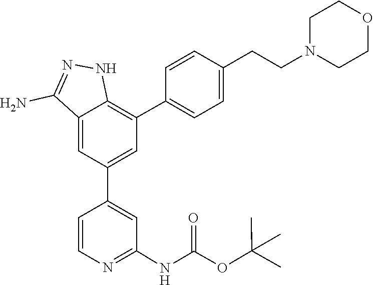 [SMILES: CC(C)(C)OC(=O)Nc1cc(-c2cc(-c3ccc(CCN4CCOCC4)cc3)c3[nH]nc(N)c3c2)ccn1]

General procedure 1 was followed using tert-butyl (4-(3-amino-7-bromo-1H-indazol-5-yl)pyridin-2-yl)carbamate (121.3 mg, 0.3 mmol), 4-(4-(4,4,5,5-tetramethyl-1,3,2-dioxaborolan-2-yl)phenethyl)morpholine (142.8 mg, 0.45 mmol), Pd₂(dppf)Cl₂ (21.9 mg, 0.03 mmol), and Cs2CO3 (293.2 mg, 0.9 mmol) in 9:1 dioxane:water at 90° C. for 16 hours. Flash column chromatography [petrol:ethyl acetate:methanol 0→100%→80%] using basic silica followed afforded the title compound (90.4 mg, 59%).

¹H NMR (400 MHz, DMSO-d₆) δ 11.69 (br s, 1H), 9.77 (br s, 1H), 8.28 (d, J=5.3 Hz, 1H), 8.15 (app. s, 2H), 7.67 (d, J=8.0 Hz, 2H), 7.58 (d, J=1.5 Hz, 1H), 7.42-7.38 (m, 2H), 7.38 (s, 1H), 5.61 (br s, 2H), 3.63-3.55 (m, 4H), 2.86-2.78 (m, 2H), 2.62-2.54 (m, 2H), 2.48-2.43 (s, 4H), 1.49 (s, 9H).

¹³C NMR (101 MHz, DMSO-d₆) δ 153.10, 152.82, 150.40, 149.93, 148.21, 140.00, 139.20, 135.00, 129.27, 128.19, 127.87, 124.23, 123.91, 118.17, 116.21, 115.90, 109.16, 79.58, 66.19, 59.95, 53.27, 32.08, 28.06.

LCMS C₂₉H₃₄N₆O₃ requires [M+H]⁺, 514.63. Observed: [M+H]⁺, 515.3.

### 5-(2-Aminopyridin-4-yl)-7-(4-(2-morpholinoethyl)phenyl)-1H-indazol-3-amine SU1772

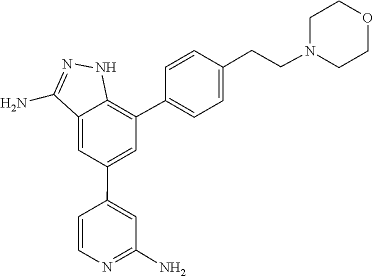 [SMILES: Nc1cc(-c2cc(-c3ccc(CCN4CCOCC4)cc3)c3[nH]nc(N)c3c2)ccn1]

To a suspension of tert-butyl (4-(3-amino-7-(4-(2-morpholinoethyl)phenyl)-1H-indazol-5-yl)pyridin-2-yl)carbamate (35 mg, 0.068 mmol) in DCM (3 mL) was added TFA (3 mL) and the mixture was stirred at room temperature for 1 hour. Excess saturated sodium carbonate solution was added, and the mixture was stirred at room temperature for 30 minutes. The mixture was diluted with DCM and water and the organic phase was separated. The aqueous layer was extracted with DCM three times and the combined organic layer was discarded. The precipitate in the aqueous layer was collected via vacuum filtration to afford the title compound (10.0 mg, 35%). ¹H NMR (500 MHz, DMSO-d₆) δ 11.62 (br s, 1H), 8.06 (s, 1H), 7.95 (d, J=5.1 Hz, 1H), 7.66 (d, J=7.9 Hz, 2H), 7.54 (s, 1H), 7.38 (d, J=7.9 Hz, 2H), 6.87 (d, J=5.1 Hz, 1H), 6.79 (s, 1H), 5.88 (br s, 2H), 5.53 (br s, 2H), 3.63-3.56 (m, 4H), 2.85-2.78 (m, 2H), 2.61-2.55 (m, 2H), 2.46 (app. br s, 4H).

¹³C NMR (101 MHz, DMSO-d₆) δ 160.42, 150.28, 148.81, 148.25, 139.94, 139.09, 135.14, 129.26, 128.73, 127.81, 124.08, 123.66, 117.59, 115.85, 110.12, 104.69, 66.21, 59.98, 53.29, 32.09.

LCMS C₂₄H₂₆N₆O requires [M+H]⁺, 414.51. Observed: [M+H]⁺, 415.3.

### Example 330—Methyl (4-(3-amino-7-(4-(2-morpholinoethyl)phenyl)-1H-indazol-5-yl)pyridin-2-yl)carbamate SU1773

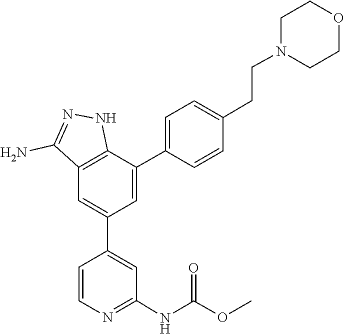 [SMILES: COC(=O)Nc1cc(-c2cc(-c3ccc(CCN4CCOCC4)cc3)c3[nH]nc(N)c3c2)ccn1]

General procedure 1 was followed using methyl (4-(3-amino-7-bromo-1H-indazol-5-yl)pyridin-2-yl)carbamate (0.5 g, 1.38 mmol, 1eq), 4-(4-(4,4,5,5-tetramethyl-1,3,2-dioxaborolan-2-yl)phenethyl)morpholine (0.48 g, 1.52 mmol, 1.1eq) Pd-DPPF (0.051 g, 0.069 mmol, 5 mol %) and cesium carbonate (1.35 g 4.14 mmol, 3.0 eq) in a sealed tube flushed with argon in a freshly degassed mixture of dioxane:water (9:1, 10 mL) and the resultant mixture stirred at 80° C. for 18 hours. The reaction was then cooled and partitioned between ethyl acetate and water.

The aqueous layer was extracted (3×20 mL) and the combined organic layers concentrated under reduced pressure and then purified by automated reverse phase flash column chromatography eluting with a gradient of 0 to 70% acetonitrile in water to afford the target product as a light brown solid (0.042 g, 6%)

¹H NMR (400 MHz, DMSO) δ 11.71 (s, 1H), 10.20 (s, 1H), 8.31 (d, J=5.3 Hz, 1H), 8.22 (s, 1H), 8.17 (s, 1H), 7.67 (d, J=7.8 Hz, 2H), 7.60 (s, 1H), 7.44 (d, J=5.5 Hz, 1H), 7.40 (d, J=7.7 Hz, 2H), 5.61 (s, 2H), 3.71 (s, 3H), 3.61 (t, J=4.6 Hz, 4H), 2.82 (d, J=7.8 Hz, 2H), 2.59 (t, J=7.8 Hz, 2H), 2.47 (s, 4H).

¹³C NMR (101 MHz, DMSO) δ 154.2, 152.9, 149.9, 148.3, 140.0, 135.0, 129.3, 128.0, 127.8, 124.2, 116.3, 109.1, 66.2, 59.9, 53.2, 51.8, 32.1.

LCMS (ESI+, m/z) expected for Chemical Formula: C₂₆H₂₈N₆O₃ is 472.5 found 473.3 (M+H), 471.3 (M−H)

### Example 331—Methyl (4-(3-amino-7-(4-(methylsulfonyl)phenyl)-1H-indazol-5-yl)pyridin-2-yl)carbamate SU1758

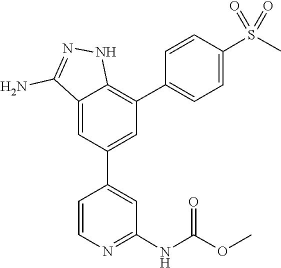 [SMILES: COC(=O)Nc1cc(-c2cc(-c3ccc(S(C)(=O)=O)cc3)c3[nH]nc(N)c3c2)ccn1]

A solution of methyl (4-(3-amino-7-bromo-1H-indazol-5-yl)pyridin-2-yl)carbamate (0.15 g, 0.4 mmol, 1 Eq), (4-(methylsulfonyl)phenyl)boronic acid (0.12 g, 0.6 mmol, 1.5 Eq), cesium carbonate (0.4 g, 1.2 mmol, 3 Eq) and [1,1′-bis(diphenylphosphino)ferrocene]dichloropalladium(II) (74 mg, 0.1 mmol, 0.25 Eq) in dioxane (2 mL) and water (0.5 mL) was stirred under nitrogen atmosphere at 80° C. for 2 h. The reaction mixture was cooled and extracted between EtOAc (10 mL) and water (2 mL). The organic layer was dried over anhydrous sodium sulphate and evaporated. The crude residue was purified using reversed-phase column chromatography (30% MeCN in H₂O) followed by HPLC purification (50% MeCN in H₂O) to give the titled product as yellow solid (10 mg, 0.02 mmol, 6%).

¹H NMR (500 MHz, DMSO) δ 11.88 (s, 1H), 10.22 (s, 1H), 8.33 (d, J=5.3 Hz, 1H), 8.28 (d, J=1.6 Hz, 1H), 8.23 (d, J=1.7 Hz, 1H), 8.06 (q, J=8.2 Hz, 4H), 7.73 (d, J=1.7 Hz, 1H), 7.48 (dd, J=5.2, 1.7 Hz, 1H), 5.71 (s, 2H), 3.71 (s, 3H), 3.31 (s, 3H).

LCMS: For C₂₁H₁₉N₅O₄S requires 437.47 found 438.1 (M+H).

### Example 332—Methyl (4-(3-amino-7-(3-hydroxyphenyl)-1H-indazol-5-yl)pyridin-2-yl)carbamate SU1748

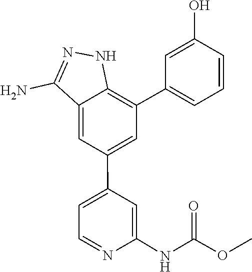 [SMILES: COC(=O)Nc1cc(-c2cc(-c3cccc(O)c3)c3[nH]nc(N)c3c2)ccn1]

A solution of methyl (4-(3-amino-7-bromo-1H-indazol-5-yl)pyridin-2-yl)carbamate (0.5 g, 1.38 mmol, 1 Eq), (3-hydroxyphenyl)boronic acid (0.29 g, 2.07 mmol, 1.5 Eq), cesium carbonate (1.4 g, 4.14 mmol, 3 Eq) and [1,1′-bis(diphenylphosphino)ferrocene]dichloropalladium(II) (152 mg, 0.21 mmol, 0.15 Eq) in dioxane (4 mL) and water (1 mL) was stirred under nitrogen atmosphere at 80° C. for 2 h. The reaction mixture was cooled and extracted between EtOAc (10 mL) and water (2 mL). The organic layer was dried over anhydrous sodium sulphate and evaporated. The crude residue was purified using reversed-phase column chromatography (40% MeCN in H₂O) to give the titled product as white solid (50 mg, 0.13 mmol, 10%).

¹H NMR (500 MHz, DMSO) δ 11.66 (s, 1H), 10.21 (s, 1H), 9.73 (s, 1H), 8.31 (d, J=5.3 Hz, 1H), 8.20 (dd, J=9.2, 1.6 Hz, 2H), 7.56 (d, J=1.7 Hz, 1H), 7.43 (dd, J=5.3, 1.7 Hz, 1H), 7.32 (t, J=8.0 Hz, 1H), 7.25 (d, J=7.5 Hz, 1H), 7.20-7.12 (m, 1H), 6.91-6.85 (m, 1H), 5.61 (s, 2H), 3.71 (s, 3H).

LC-MS: For C₂₀H₁₇N₅O₃ requires 375.39 found 376.3 (M+H).

### Example 333—Methyl (4-(3-amino-7-(3-carbamoylphenyl)-1H-indazol-5-yl)pyridin-2-yl)carbamate SU1754

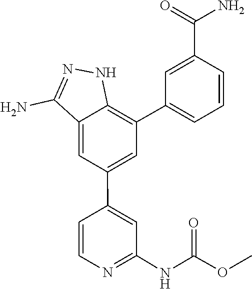 [SMILES: COC(=O)Nc1cc(-c2cc(-c3cccc(C(N)=O)c3)c3[nH]nc(N)c3c2)ccn1]

A solution of methyl (4-(3-amino-7-bromo-1H-indazol-5-yl)pyridin-2-yl)carbamate (0.15 g, 0.4 mmol, 1 Eq), 3-(4,4,5,5-tetramethyl-1,3,2-dioxaborolan-2-yl)benzamide (0.15 g, 0.6 mmol, 1.5 Eq), cesium carbonate (0.4 g, 1.2 mmol, 3 Eq) and [1,1′-bis(diphenylphosphino)ferrocene]dichloropalladium(II) (74 mg, 0.1 mmol, 0.25 Eq) in dioxane (2 mL) and water (0.5 mL) was stirred under nitrogen atmosphere at 80° C. for 18 h. The reaction mixture was cooled and extracted between EtOAc (10 mL) and water (2 mL). The organic layer was dried over anhydrous sodium sulphate and evaporated. The crude residue was purified using reversed-phase column chromatography (40% MeCN in H₂O) followed by HPLC purification (50% MeCN in H₂O) to give the titled product as yellow solid (30 mg, 0.07 mmol, 18%).

¹H NMR (500 MHz, DMSO) δ 11.82 (s, 1H), 10.20 (s, 1H), 8.32 (d, J=5.2 Hz, 1H), 8.21 (t, J=1.7 Hz, 2H), 8.19-8.12 (m, 2H), 7.90 (dd, J=7.6, 1.7 Hz, 2H), 7.68 (d, J=1.7 Hz, 1H), 7.63 (t, J=7.7 Hz, 1H), 7.48-7.43 (m, 2H), 5.66 (s, 2H), 3.71 (s, 3H).

LCMS: For C₂₁H₁₈N₆O₃ requires 402.41 found 403.3 (M+H).

### Example 334—Methyl (4-(3-amino-7-(3-(morpholinomethyl)phenyl)-1H-indazol-5-yl)pyridin-2-yl)carbamate SU1765

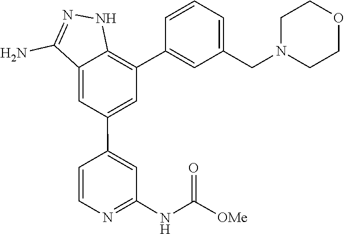 [SMILES: COC(=O)Nc1cc(-c2cc(-c3cccc(CN4CCOCC4)c3)c3[nH]nc(N)c3c2)ccn1]

General procedure 1 was followed using methyl (4-(3-amino-7-bromo-1H-indazol-5-yl)pyridin-2-yl)carbamate (36.2 mg, 0.1 mmol), (3-(morpholinomethyl)phenyl)boronic acid (44.2 mg, 0.2 mmol), Pd₂(dppf)Cl₂ (7.3 mg, 0.01 mmol), and Cs₂CO3 (97.7 mg, 0.3 mmol) in 9:1 dioxane:water at 100° C. for 16 hours. Flash column chromatography [petrol:THF 0→100%] using basic silica followed by trituration from methanol afforded the title compound (7 mg, 15%).

¹H NMR (400 MHz, DMSO-d₆) δ 11.72 (br s, 1H), 10.21 (br s, 1H), 8.31 (d, J=5.3 Hz, 1H), 8.22 (d, J=1.2 Hz, 1H), 8.19 (d, J=1.5 Hz, 1H), 7.67 (s, 1H), 7.64 (app. d, J=7.7 Hz, 1H), 7.61 (d, J=1.5 Hz, 1H), 7.49 (app. t, J=7.7 Hz, 1H), 7.44 (dd, J=5.3, 1.2 Hz, 1H), 7.38 (app. d, J=7.7 Hz, 1H), 5.63 (br s, 2H), 3.70 (s, 3H), 3.63-3.53 (m, 7H), 2.46-2.33 (m, 5H).

¹³C NMR (101 MHz, DMSO-d₆) δ 154.19, 152.92, 150.47, 149.93, 148.30, 139.20, 138.69, 137.22, 128.88, 128.50, 128.27, 128.03, 124.35, 123.98, 118.41, 116.37, 115.93, 109.08, 66.21, 62.36, 53.25, 51.80.

LCMS C₂₅H₂₆N₆O₃ requires [M+H]⁺, 458.21. Observed: [M+H]⁺, 459.3.

### Example 335—N-(4-(3-Amino-1H-indazol-5-yl)pyridin-2-yl)-2-cyclohexylacetamide SU1340

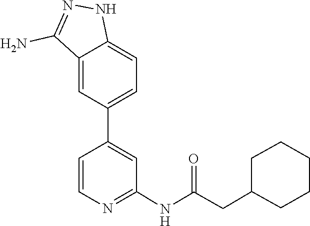 [SMILES: Nc1n[nH]c2ccc(-c3ccnc(NC(=O)CC4CCCCC4)c3)cc12]

To a suspension of N-(4-chloropyridin-2-yl)-2-cyclohexylacetamide (0.307 g, 1.2 mmol), 5-(4,4,5,5-tetramethyl-1,3,2-dioxaborolan-2-yl)-1H-indazol-3-amine (0.362 g, 1.4 mmol) and [1,1′-bis(di-tert-butylphosphino)ferrocene]dichloropalladium(II) catalyst (0.063 g, 0.097 mmol) in EtOH (3.0 mL, degassed under nitrogen) was added K₃PO₄ (1M, 1.2 mL) and the reaction mixture was heated to 80° C. for 24 h. The reaction mixture was cooled to room temperature and diluted with EtOAc, washed with water, aqueous saturated NaHCO₃ and brine. The organics were concentrated under reduced pressure and the resulting residue and purified by column chromatography (100% Hexane—1/1 Hexane/EtOAc—100% EtOAc) to afford the title compound as an off-white solid (0.061 g, 0.17 mmol, 42%).

¹H NMR (DMSO-d₆): δ 0.99 (m, 2H), 1.22 (m, 3H), 1.62 (m, 2H), 1.69 (m, 4H), 1.79 (m, 1H), 2.30 (d, J=5.6 Hz, 2H), 7.35 (d, J=7.2 Hz, 1H), 7.37 (dd, J=1.2, 4.0 Hz, 1H), 7.59 (dd, J=1.2, 6.8 Hz, 1H), 8.16 (s, 1H), 8.31 (d, J=4.4 Hz, 1H), 8.46 (s, 1H), 10.41 (br s, 1H), 11.56 (br s, 1H).

HRMS: For C₂₀H₂₄ON₅ requires 350.1975 found 350.1795. (M+H)⁺

### Example 336—6-Amino-4-(3-amino-7-(3,3-dimethylbut-1-yn-1-yl)-1H-indazol-5-yl)nicotinonitrile SU1710

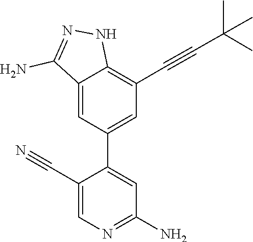 [SMILES: CC(C)(C)C#Cc1cc(-c2cc(N)ncc2C#N)cc2c(N)n[nH]c12]

A solution of 7-(3,3-dimethylbut-1-yn-1-yl)-5-(4,4,5,5-tetramethyl-1,3,2-dioxaborolan-2-yl)-1H-indazol-3-amine (0.5 g, 1.47 mmol, 1 Eq), 6-amino-4-chloronicotinonitrile (0.23 g, 1.47 mmol, 1 Eq), cesium carbonate (1.4 g, 4.41 mmol, 3 Eq) and 1,1′-bis(di-tert-butylphosphino) ferrocene palladium chloride (144 mg, 0.22 mmol, 0.15 Eq) in dioxane (4 mL) and water (1 mL) was stirred under nitrogen atmosphere at 70° C. for 18 h. The reaction mixture was cooled and extracted between EtOAc (10 mL) and water (5 mL). The organic layer was washed with brine (2×5 mL), dried over anhydrous Na₂SO₄ and evaporated under reduced pressure. The crude residue was purified using column chromatography (80% EtOAc in petroleum ether) to give the titled product as yellow solid (0.4 g, 1.2 mmol, 82%).

¹H NMR (500 MHz, DMSO) δ 1.37 (s, 9H), 5.64 (s, 2H), 6.54 (s, 1H), 7.05 (s, 2H), 7.35 (d, J=1.7 Hz, 1H), 7.93 (d, J=1.7 Hz, 1H), 8.40 (s, 1H), 11.89 (s, 1H).

LCMS: For C₁₉H₁₈N₆ requires 330.4 found 331.3 (M+H).

### Biological Assays

### IKK Assays

IKKα and IKKβ inhibitory activity was determined using three different assays: a dissociation enhanced ligand fluorescent immunoassay (DELFIA) based on the protocol of HTScan™ IKKβ Kinase Assay (Cell Signaling Technology, USA); an Adapta IKKα assay (performed by Life Technologies); a Z′-LYTE™ IKKβ assay (performed by Life Technologies).

### The DELFIA Assay

Recombinant IKKα or IKKβ, 37 nM, (Millipore, Dundee, UK) was incubated with IηB-α (Ser32) (New England Biolabs, Hitchin, UK) biotinylated peptide substrate (0.375 μM) and 40 μM ATP in assay buffer (40 mM Tris-HCl (pH 7.5), 20 mM MgCl₂, EDTA 1 mM, DTT 2 mM and BSA 0.01 mg/ml) in a V-well 96-well plate in the presence and absence of test compound. The assay plate was incubated for 60 minutes at 30° C. after which the kinase reaction was quenched by the addition of 50 mM EDTA, pH8. The resulting mixture was transferred to a streptavidin coated 96-well plate (Perkin Elmer, Beaconsfield, UK) and incubated for 1 hour at 30° C. to immobilise the substrate peptide. After three washes with wash buffer (0.01 M phosphate buffered saline (PBS), 0.05% Tween −20, pH 7.4) a primary antibody against the phosphorylated substrate (phospho-IηB-a) (Ser32/36) (5A5) Mouse mAb (New England Biolabs, Hitchin, UK) (1:1000 dilution with 1% bovine serum albumin (BSA) in wash buffer) was added and incubated at 37° C. for 2 h.

After a further three washes, a secondary europiated antibody (Eu-N1 labelled anti-mouse IgG, (Perkin Elmer, Beaconsfield, UK) diluted 1:500 in 1% BSA/wash buffer) was added and incubated at 30° C. for 30 minutes. After a further five washes, DELFIA enhancement solution—(Perkin Elmer, Beaconsfield UK) was added and allowed to incubate for 10 min at room temperature, protected from light, to facilitate the chemifluorescent detection. The relative fluorescence units (RFU) signal were measured on a Wallac Victor 1420 multilabel counter (Perkin Elmer, Beaconsfield, UK), in time-resolved fluorescence mode. The counter was set at an excitation wavelength of 340 nm with a 400 μs delay before detecting emitted light at 615 nm. The apparent Kᵢ of the phosphorylated substrate was calculated for each compound using the Cheng-Prusoff Equation.

Adapta IKKα assay

The reaction was performed in low-volume white 384-well plates (Corning model 4512).

The test compounds were screened in 1% DMSO (final) in the well. For 10 point titrations, 3-fold serial dilutions were conducted from the starting concentration of 50 mM.

The 2× CHUK (IKK alpha) kinase was prepared in 50 mM HEPES pH 7.5, 0.01% BRIJ-35, 10 mM MgCl₂ and 1 mM EGTA. The final 10 μL kinase mixture consisted of 50-250 ng CHUK (IKK alpha) in 32.5 mM HEPES pH 7.5, 0.005% BRIJ-35, 5 mM MgCl₂ and 0.5 mM EGTA. No substrate was required, as the assay measures the ability of a compound to inhibit the kinase's intrinsic ATPase activity.

5 μL of 2× Kinase mixture was incubated with 2.5 μL of ATP solution diluted to a 4×working concentration in water, in the presence of 100 nL of 100× test compound in 100% DMSO and 2.4 μL of 30 mM HEPES buffer.

After 30-second plate shake and 1-minute centrifuge at 1000×g, the resulting kinase reaction was allowed to incubate for 1 hour at room temperature before 5 μL of Detection Mix was added.

The Detection Mix, previously prepared in TR-FRET Dilution Buffer, consisted of EDTA (30 mM), Eu-anti-ADP antibody (6 nM) and an Alexa Fluor™ 647 labelled ADP tracer. The Detection Mix contained the EC₆₀ concentration of tracer for 5-150 μM ATP.

After 30-second plate shake and 1-minute centrifuge at 1000×g, the Detection Mix was allowed to equilibrate at room temperature for 1 hour. Then the plate was read using a fluorescence plate reader configured for Adapta™ TR-FRET and the data analysed.

ADP formation was determined by calculating the emission ratio from the assay well. Therefore, the emission ratio was calculated by dividing the intensity of the tracer (acceptor) emission by the intensity of the Eu (donor) emission at 615 nm as shown in the equation below.

$$Emission Ratio = \frac{AlexaFluor 647 Emission ( 665 nm )}{Europium Emission ( 615 nm )}$$

### Z′-LYTE™ IKKβ Assay

The reaction was performed in low volume NBS, black 384-well plates (Corning model 4514).

The test compounds were screened in 1% DMSO (final) in the well. For 10 point titrations, 3-fold serial dilutions were conducted from the starting concentration of 50 mM.

The 2× IKBKB (IKKβ)/Ser/Thr 05 mixture was prepared in 50 mM HEPES pH 7.5, 0.01% BRIJ-35, 10 mM MgCl₂, 1 mM EGTA. The final 10 μL Kinase Reaction consisted of 0.62-4 ng IKBKB (IKKβ) and 2 μM Ser/Thr 05 in 50 mM HEPES pH 7.5, 0.01% BRIJ-35, 10 mM MgCl₂, 1 mM EGTA.

5 μL of 2× Kinase reaction was incubated with 2.5 μL of ATP solution diluted to a 4× working concentration in Kinase Buffer (50 mM HEPES pH 7.5, 0.01% BRIJ-35, 10 mM MgCl₂, 1 mM EGTA) in the presence of 00 nL of 100× test compound in 100% DMSO and 2.4 μL of Kinase buffer.

The resulting kinase reaction was allowed to incubate for 1 hour at room temperature before 5 μL of a 1:64 dilution of Development Reagent B was added and the resulting Development Reaction was incubated for another hour at room temperature. Then the plate was read on a fluorescence plate reader and the data analysed.

A ratiometric method, which calculated the Emission Ratio of donor emission (i.e. coumarin) to acceptor emission (i.e. fluorescein) after excitation of the donor fluorophore at 400 nm, was used to quantitate reaction progress, as shown in the equation below.

$$Emission Ratio = \frac{Coumarin Emission ( 445 nm )}{Fluorescein Emission ( 520 nm )}$$

### Biological Data

| | | | IKKα | | IKKβ | |
|---|---|---|---|---|---|---|
| Example | SU ID | IUPAC Name | (nM) | | (nM) | |
| 1 | SU1407 | 5-(2-aminopyridin-4-yl)-7-chloro-1H-indazol-3- | 55 | | 880 | |
| | | amine | | | | |
| 2 | SU1112 | 5-(2-aminopyridin-4-yl)-7-methyl-1H-indazol- | 72 | | 461 | |
| | | 3-amine | | | | |
| 3 | SU1581 | 5-(2-aminopyridin-4-yl)-7-(trifluoromethyl)-1H- | 38 | | 194 | |
| | | indazol-3-amine | | | | |
| 4 | SU1397 | 7-chloro-5-(1H-pyrrolo[2,3-b]pyridin-4-yl)-1H- | 4 | | 93 | |
| | | indazol-3-amine | | | | |
| 5 | SU1481 | 7-bromo-5-(1H-pyrrolo[2,3-b]pyridin-4-yl)-1H- | 6 | | 26 | |
| | | indazol-3-amine | | | | |
| 6 | SU1492 | 7-ethynyl-5-(1H-pyrrolo[2,3-b]pyridin-4-yl)-1H- | 26 | | 137 | |
| | | indazol-3-amine | | | | |
| 7 | SU1398 | 7-phenyl-5-(1H-pyrrolo[2,3-b]pyridin-4-yl)-1H- | 35 | | 1257 | |
| | | indazol-3-amine | | | | |
| 8 | SU1265 | 5-(2-methyl-1H-pyrrolo[2,3-b]pyridin-4-yl)-1H- | 2 | | 33 | |
| | | indazol-3-amine | | | | |
| 9 | SU1457 | 5-(2-(tert-butyl)-1H-pyrrolo[2,3-b]pyridin-4-yl)- | 19 | | 109 | |
| | | 1H-indazol-3-amine | | | | |
| 10 | SU1291 | 4-(3-amino-1H-indazol-5-yl)-1H-pyrrolo[2,3- | 5 | | 335 | |
| | | b]pyridine-2-carboxylic acid | | | | |
| 11 | SU1482 | 7-bromo-5-(7H-pyrrolo[2,3-d]pyrimidin-4-yl)- | 39 | | 258 | |
| | | 1H-indazol-3-amine | | | | |
| 12 | SU1078 | 5-(2-(ethylamino)pyridin-4-yl)-1H-indazol-3- | 8 | | 38 | |
| | | amine | | | | |
| 13 | SU1101 | 5-(2-(propylamino)pyridin-4-yl)-1H-indazol-3- | 49 | | 251 | |
| | | amine | | | | |
| 14 | SU1134 | 5-(2-(isopropylamino)pyridin-4-yl)-1H-indazol- | 76 | | 587 | |
| | | 3-amine | | | | |
| 15 | SU1127 | 5-(2-((cyclopropylmethyl)amino)pyridin-4-yl)- | 171 | | 796 | |
| | | 1H-indazol-3-amine | | | | |
| 16 | SU1135 | 5-(2-(isopentylamino)pyridin-4-yl)-1H-indazol- | 65 | | 838 | |
| | | 3-amine | | | | |
| 17 | SU1122 | 5-(2-(hexylamino)pyridin-4-yl)-1H-indazol-3- | 114 | | 1341 | |
| | | amine | | | | |
| 18 | SU1126 | 5-(2-(cyclohexylamino)pyridin-4-yl)-1H- | 571 | | >10000 | |
| | | indazol-3-amine | | | | |
| 19 | SU1224 | 5-{2-[(Trans-4-methylcyclohexyl)amino]pyridin-4- | 170 | | >10000 | |
| | | yl}-1H-indazol-3-amine | | | | |
| 20 | SU1103 | 2-((4-(3-amino-1H-indazol-5-yl)pyridin-2- | 46 | | 630 | |
| | | yl)amino)ethan-1-ol | | | | |
| 21 | SU1104 | 3-((4-(3-amino-1H-indazol-5-yl)pyridin-2- | 15 | | 796 | |
| | | yl)amino)propan-1-ol | | | | |
| 22 | SU1144 | 4-((4-(3-amino-1H-indazol-5-yl)pyridin-2- | 22 | | 1467 | |
| | | yl)amino)butan-1-ol | | | | |
| 23 | SU1155 | 5-((4-(3-amino-1H-indazol-5-yl)pyridin-2- | 33 | | 335 | |
| | | yl)amino)pentan-1-ol | | | | |
| 24 | SU1179 | 5-{2-[(trans-4-hydroxycyclohexyl)amino]pyridin-4- | 100 | | >10000 | |
| | | yl}-1H-indazol-3-amine I | | | | |
| 25 | SU1125 | 5-(2-((2-methoxyethyl)amino)pyridin-4-yl)-1H- | 221 | | >10000 | |
| | | indazol-3-amine | | | | |
| 26 | SU1142 | 5-(2-((3-methoxypropyl)amino)pyridin-4-yl)- | 34 | | 754 | |
| | | 1H-indazol-3-amine | | | | |
| 27 | SU1196 | 5-(2-((3-isopropoxypropyl)amino)pyridin-4-yl)- | 76 | | 2514 | |
| | | 1H-indazol-3-amine | | | | |
| 28 | SU1177 | 3-((4-(3-amino-1H-indazol-5-yl)pyrimidin-2- | 65 | | 460 | |
| | | yl)amino)propan-1-ol | | | | |
| 29 | SU1145 | 3-((4-(3-amino-1H-indazol-5-yl)pyridin-2- | 1900 | | >10000 | |
| | | yl)(methyl)amino)propan-1-ol | | | | |
| 30 | SU1096 | 5-(2-((2-morpholinoethyl)amino)pyridin-4-yl)- | 340 | | 377 | |
| | | 1H-indazol-3-amine | | | | |
| 31 | SU1119 | 5-(2-((2-(piperidin-1-yl)ethyl)amino)pyridin-4- | 495 | | >10000 | |
| | | yl)-1H-indazol-3-amine | | | | |
| 32 | SU1153 | N¹-(4-(3-amino-1H-indazol-5-yl)pyridin-2-yl)- | 340 | | >10000 | |
| | | N3-methylpropane-1,3-diamine | | | | |
| 33 | SU1084 | N-(4-(3-amino-1H-indazol-5-yl)pyridin-2- | 76 | | 159 | |
| | | yl)cyclopropanecarboxamide | | | | |
| 34 | SU1247 | N-(4-(3-amino-1H-indazol-5-yl)pyridin-2- | 26 | | 350 | |
| | | yl)benzamide | | | | |
| 35 | SU1181 | ethyl (4-(3-amino-1H-indazol-5-yl)pyridine-2- | 15 | | 470 | |
| | | yl)carbamate | | | | |
| 36 | SU1157 | 1-(4-(3-amino-1H-indazol-5-yl)pyridine-2-yl)- | 20 | | 838 | |
| | | 3-ethylurea | | | | |
| 37 | SU1182 | 1-(4-(3-amino-1H-indazol-5-yl)pyridine-2-yl)- | 70 | | 4100 | |
| | | 3-propylurea | | | | |
| 38 | SU1185 | 1-(4-(3-amino-1H-indazol-5-yl)pyridine-2-yl)- | 100 | | 4200 | |
| | | 3-isopentylurea | | | | |
| 39 | SU1188 | 1-(4-(3-amino-1H-indazol-5-yl)pyridine-2-yl)- | 114 | | 970 | |
| | | 3-cyclopentylurea | | | | |
| 40 | SU1189 | 1-(4-(3-amino-1H-indazol-5-yl)pyridine-2-yl)- | 228 | | >10000 | |
| | | 3-cyclohexylurea | | | | |
| 41 | SU1180 | 1-(4-(3-amino-1H-indazol-5-yl)pyridine-2-yl)- | 21 | | 754 | |
| | | 3-(2-hydroxyethyl)urea | | | | |
| 42 | SU1183 | 1-(4-(3-amino-1H-indazol-5-yl)pyridine-2-yl)- | 114 | | 670 | |
| | | 3-(3-hydroxypropyl)urea | | | | |
| 43 | SU1193 | 1-(4-(3-amino-1H-indazol-5-yl)pyridine-2-yl)- | 76 | | 1592 | |
| | | 3-(2-methoxyethyl)urea | | | | |
| 44 | SU1198 | 3-(4-(3-amino-1H-indazol-5-yl)pyridine-2-yl)- | 418 | | >10000 | |
| | | 1-(2-hydroxyethyl)-1-methylurea | | | | |
| 45 | SU1190 | 1-(4-(3-amino-1H-indazol-5-yl)pyridine-2-yl)- | 152 | | 357 | |
| | | 3-benzylurea | | | | |
| 46 | SU1194 | 1-(4-(3-amino-1H-indazol-5-yl)pyridine-2-yl)- | 571 | | >10000 | |
| | | 3-phenethylurea | | | | |
| 47 | SU1184 | 1-(4-(3-amino-1H-indazol-5-yl)pyridine-2-yl)- | 38 | | 838 | |
| | | 3-(pyridine-2-ylmethyl)urea | | | | |
| 48 | SU1186 | 1-(4-(3-amino-1H-indazol-5-yl)pyridine-2-yl)- | 76 | | 840 | |
| | | 3-(pyridine-3-ylmethyl)urea | | | | |
| 49 | SU1199 | 1-(4-(3-amino-1H-indazol-5-yl)pyridine-2-yl)- | 304 | | 1844 | |
| | | 3-(pyridine-4-ylmethyl)urea | | | | |
| 50 | SU1217 | 1-(4-(3-amino-1H-indazol-5-yl)pyridine-2-yl)- | 12 | | 293 | |
| | | 3-phenylurea | | | | |
| 51 | SU1251 | 1-(4-(3-amino-1H-indazol-5-yl)pyridine-2-yl)- | 14 | | 126 | |
| | | 3-(3-fluorophenyl)urea | | | | |
| 52 | SU1219 | 1-(4-(3-amino-1H-indazol-5-yl)pyridine-2-yl)- | 36 | | 400 | |
| | | 3-(3-chlorophenyl)urea | | | | |
| 53 | SU1230 | 1-(4-(3-amino-1H-indazol-5-yl)pyridine-2-yl)- | 14 | | 419 | |
| | | 3-(3-isopropylphenyl)urea | | | | |
| 54 | SU1204 | 1-(4-(3-amino-1H-indazol-5-yl)pyridine-2-yl)- | 14 | | 419 | |
| | | 3-(3-(hydroxymethyl)phenyl)urea | | | | |
| 55 | SU1228 | 3-(3-(4-(3-amino-1H-indazol-5-yl)pyridine-2- | 21 | | 503 | |
| | | yl)ureido)benzamide | | | | |
| 56 | SU1237 | 1-(4-(3-amino-1H-indazol-5-yl)pyridine-2-yl)- | 237 | | 3500 | |
| | | 3-(3-phenoxyphenyl)urea | | | | |
| 57 | SU1231 | 1-(4-(3-amino-1H-indazol-5-yl)pyridine-2-yl)- | 38 | | 4200 | |
| | | 3-(3-(benzyloxy)phenyl)urea | | | | |
| 58 | SU1270 | 1-(4-(3-amino-1H-indazol-5-yl)pyridine-2-yl)- | 80 | | 1970 | |
| | | 3-(3-((4-fluorobenzyl)oxy)phenyl)urea | | | | |
| 59 | SU1272 | 1-(4-(3-amino-1H-indazol-5-yl)pyridine-2-yl)- | 93 | | 1781 | |
| | | 3-(3-((3-fluorobenzyl)oxy)phenyl)urea | | | | |
| 60 | SU1274 | 1-(4-(3-amino-1H-indazol-5-yl)pyridine-2-yl)- | 76 | | 2479 | |
| | | 3-(3-((2-fluorobenzyl)oxy)phenyl)urea | | | | |
| 61 | SU1277 | 3-(3-(4-(3-amino-1H-indazol-5-yl)pyridine-2- | 171 | | 419 | |
| | | yl)ureido)-N-phenylbenzamide | | | | |
| 62 | SU1238 | 1-(4-(3-amino-1H-indazol-5-yl)pyridine-2-yl)- | 22 | | 115 | |
| | | 3-(4-fluorophenyl)urea | | | | |
| 63 | SU1226 | 1-(4-(3-amino-1H-indazol-5-yl)pyridine-2-yl)- | 48 | | 1718 | |
| | | 3-(4-chlorophenyl)urea | | | | |
| 64 | SU1227 | 1-(4-(3-amino-1H-indazol-5-yl)pyridine-2-yl)- | 76 | | 2900 | |
| | | 3-(4-(tert-butyl)phenyl)urea | | | | |
| 65 | SU1218 | 1-(4-(3-amino-1H-indazol-5-yl)pyridine-2-yl)- | 27 | | 419 | |
| | | 3-(4-(methylsulfonyl)phenyl)urea | | | | |
| 66 | SU1258 | 1-(4-(3-amino-1H-indazol-5-yl)pyridin-2-yl)-3- | 46 | | 1050 | |
| | | (o-tolyl)urea | | | | |
| 67 | SU1263 | 1-(4-(3-amino-1H-indazol-5-yl)pyridine-2-yl)- | 300 | | 4200 | |
| | | 3-(2-ethylphenyl)urea | | | | |
| 68 | SU1271 | 1-(4-(3-amino-1H-indazol-5-yl)pyridine-2-yl)- | 100 | | 1900 | |
| | | 3-(2-isopropylphenyl)urea | | | | |
| 69 | SU1225 | 1-(4-(3-amino-1H-indazol-5-yl)pyridine-2-yl)- | 42 | | 1190 | |
| | | 3-(pyridine-3-yl)urea | | | | |
| 70 | SU1677 | 5-(2-amino-3-ethynylpyridin-4-yl)-1H-indazol- | | | | |
| | | 3-amine | | | | |
| 71 | SU1673 | 5-(2-amino-3-(cyclopropylethynyl)pyridin-4- | | | | |
| | | yl)-1H-indazol-3-amine | | | | |
| 72 | SU1499 | 5-(2-amino-3-(3,3-dimethylbut-1-yn-1- | 6 | | 268 | |
| | | yl)pyridine-4-yl)-1H-indazol-3-amine | 1 | | 65 | |
| 73 | SU1536 | 5-(2-amino-3-(cyclopentylethynyl)pyridine-4- | 12 | | 289 | |
| | | yl)-1H-indazol-3-amine | 2 | | 206 | |
| 74 | SU1493 | 5-(2-amino-3-(cyclohexylethynyl)pyridine-4- | 12 | | 342 | |
| | | yl)-1H-indazol-3-amine | 3 | | 342 | |
| 75 | SU1539 | 5-(2-amino-3-(phenylethynyl)pyridine-4-yl)- | 10 | | 152 | |
| | | 1H-indazol-3-amine | 1 | | 101 | |
| 76 | SU1594 | 5-(2-amino-3-((4- | 8 | | 32 | |
| | | aminophenyl)ethynyl)pyridine-4-yl)-1H- | | | | |
| | | indazol-3-amine | | | | |
| 77 | SU1593 | 5-(2-amino-3-((3- | 4 | | 22 | |
| | | aminophenyl)ethynyl)pyridine-4-yl)-1H- | | | | |
| | | indazol-3-amine | | | | |
| 78 | SU1598 | 5-(2-amino-3-((2- | 17 | | 59 | |
| | | aminophenyl)ethynyl)pyridine-4-yl)-1H- | | | | |
| | | indazol-3-amine | | | | |
| 79 | SU1596 | methyl 3-((2-amino-4-(3-amino-1H-indazol-5- | 17 | | 128 | |
| | | yl)pyridine-3-yl)ethynyl)benzoate | | | | |
| 80 | SU1597 | methyl 4-((2-amino-4-(3-amino-1H-indazol-5- | 2 | | 15 | |
| | | yl)pyridine-3-yl)ethynyl)benzoate | | | | |
| 81 | SU1595 | 5-(2-amino-3-((2- | 15 | | 484 | |
| | | methoxyphenyl)ethynyl)pyridine-4-yl)-1H- | | | | |
| | | indazol-3-amine | | | | |
| 82 | SU1591 | 5-(2-amino-4-(3-amino-1H-indazol-5- | 19 | | 537 | |
| | | yl)pyridine-3-yl)-1-phenylpent-4-yn-1-one | | | | |
| 83 | SU1515 | 3-(2-amino-4-(3-amino-1H-indazol-5- | 6 | | 402 | |
| | | yl)pyridine-3-yl)prop-2-yn-1-ol | 1 | | 36 | |
| 84 | SU1503 | 4-(2-amino-4-(3-amino-1H-indazol-5- | 1 | | 103 | |
| | | yl)pyridine-3-yl)but-3-yn-1-ol | 1 | | 53 | |
| 85 | SU1495 | 5-(2-amino-4-(3-amino-1H-indazol-5- | 4 | | 96 | |
| | | yl)pyridine-3-yl)pent-4-yn-1-ol | 1 | | 25 | |
| 86 | SU1590 | 6-(2-amino-4-(3-amino-1H-indazol-5- | 28 | | 390 | |
| | | yl)pyridine-3-yl)-2-methylhex-5-yn-2-ol | | | | |
| 87 | SU1497 | 4-(2-amino-4-(3-amino-1H-indazol-5- | 8 | | 294 | |
| | | yl)pyridine-3-yl)-2-methylbut-3-yn-2-ol | 4 | | 36 | |
| 88 | SU1534 | 5-(2-amino-3-(3-(tert-butoxy)prop-1-yn-1- | 133 | | 2340 | |
| | | yl)pyridine-4-yl)-1H-indazol-3-amine | 8 | | 442 | |
| 89 | SU1537 | 1-((2-amino-4-(3-amino-1H-indazol-5- | | | | |
| | | yl)pyridine-3-yl)ethynyl)cyclopentan-1-ol | | | | |
| 90 | SU1531 | 1-((2-amino-4-(3-amino-1H-indazol-5- | 36 | | 1406 | |
| | | yl)pyridine-3-yl)ethynyl)cyclohexan-1-ol | 3 | | 205 | |
| 91 | SU1532 | 1-((2-amino-4-(3-amino-1H-indazol-5- | 105 | | 3087 | |
| | | yl)pyridine-3-yl)ethynyl)cycloheptan-1-ol | 7 | | 505 | |
| 92 | SU1501 | 5-(2-amino-3-(3-amino-3-methylbut-1-yn-1- | 7 | | 325 | |
| | | yl)pyridin-4-yl)-1H-indazol-3-amine | 6 | | 87 | |
| 93 | SU1509 | 5-(2-amino-3-(4-(piperidin-1-yl)but-1-yn-1- | 32 | | 880 | |
| | | yl)pyridin-4-yl)-1H-indazol-3-amine | 7 | | 254 | |
| 94 | SU1510 | 5-(2-amino-3-(4-morpholinobut-1-yn-1- | 38 | | 922 | |
| | | yl)pyridin-4-yl)-1H-indazol-3-amine | | | | |
| 95 | SU1508 | 5-(2-amino-3-(5-(piperidin-1-yl)pent-1-yn-1- | 91 | | 3270 | |
| | | yl)pyridin-4-yl)-1H-indazol-3-amine | 12 | | 545 | |
| 96 | SU1490 | 5-(2-amino-3-(5-morpholinopent-1-yn-1- | 350 | | 1151 | |
| | | yl)pyridin-4-yl)-1H-indazol-3-amine | 21 | | 705 | |
| 97 | SU1601 | 5-(2-amino-3-(3-(piperidin-4-yl)prop-1-yn-1- | 15 | | 105 | |
| | | yl)pyridin-4-yl)-1H-indazol-3-amine | | | | |
| 98 | SU1538 | 3-(2-amino-4-(3-amino-1H-indazol-5- | >10,000 | | >10,000 | |
| | | yl)pyridin-3-yl)-N-methylpropiolamide | | | | |
| 99 | SU1588 | 5-(2-amino-4-(3-amino-1H-indazol-5- | 15 | | 531 | |
| | | yl)pyridine-3-yl)-1-morpholinopent-4-yn-1-one | 3 | | 264 | |
| 100 | SU1523 | 5-(2-amino-3-cyclopropylpyridin-4-yl)-1H- | 1788 | | >10000 | |
| | | indazol-3-amine | | | | |
| 101 | SU1664 | 4-(2-amino-4-(3-amino-1H-indazol-5- | 5580 | | | |
| | | yl)pyridin-3-yl)butan-1-ol | | | | |
| 102 | SU1665 | 1-(2-(2-amino-4-(3-amino-1H-indazol-5- | 8980 | | | |
| | | yl)pyridin-3-yl)ethyl)cyclohexan-1-ol | | | | |
| 103 | SU1671 | 5-(2-amino-4-(3-amino-1H-indazol-5- | 24800 | | | |
| | | yl)pyridin-3-yl)pentan-1-ol | | | | |
| 104 | SU1411 | 5-(2-aminopyridin-4-yl)-7-phenyl-1H-indazol- | 54 | | 838 | |
| | | 3-amine | 3 | | 162 | |
| 105 | SU1444 | 5-(2-aminopyridin-4-yl)-7-(3-fluorophenyl)-1H- | 40 | | 500 | |
| | | indazol-3-amine | | | | |
| 106 | SU1412 | 5-(2-aminopyridin-4-yl)-7-(3- | 162 | | 1006 | |
| | | (trifluoromethyl)phenyl)-1H-indazol-3-amine | | | | |
| 107 | SU1437 | 7-(3-aminophenyl)-5-(2-aminopyridin-4-yl)- | 14 | | 545 | |
| | | 1H-indazol-3-amine | | | | |
| 108 | SU1415 | 3-(3-amino-5-(2-aminopyridin-4-yl)-1H- | 10 | | 680 | |
| | | indazol-7-yl)phenol | | | | |
| 109 | SU1413 | 5-(2-aminopyridin-4-yl)-7-(3-methoxyphenyl)- | 55 | | 587 | |
| | | 1H-indazol-3-amine | | | | |
| 110 | SU1424 | (3-(3-amino-5-(2-aminopyridin-4-yl)-1H- | 29 | | 290 | |
| | | indazol-7-yl)phenyl)methanol | | | | |
| 111 | SU1423 | 3-(3-amino-5-(2-aminopyridin-4-yl)-1H- | 12 | | 100 | |
| | | indazol-7-yl)benzaldehyde | | | | |
| 112 | SU1422 | ethyl 3-(3-amino-5-(2-aminopyridin-4-yl)-1H- | 152 | | 1467 | |
| | | indazol-7-yl)benzoate | | | | |
| 113 | SU1445 | 3-(3-amino-5-(2-aminopyridin-4-yl)-1H- | 43 | | 840 | |
| | | indazol-7-yl)benzamide | | | | |
| 114 | SU1429 | 3-(3-amino-5-(2-aminopyridin-4-yl)-1H- | 7 | | 461 | |
| | | indazol-7-yl)benzenesulfonamide | | | | |
| 115 | SU1421 | 5-(2-aminopyridin-4-yl)-7-(3- | 79 | | 2000 | |
| | | (methylsulfonyl)phenyl)-1H-indazol-3-amine | | | | |
| 116 | SU1414 | 5-(2-aminopyridin-4-yl)-7-(3- | 162 | | 1006 | |
| | | (morpholinomethyl)phenyl)-1H-indazol-3- | | | | |
| | | amine | | | | |
| 117 | SU1416 | 4-(3-amino-5-(2-aminopyridin-4-yl)-1H- | 17 | | 235 | |
| | | indazol-7-yl)phenol | | | | |
| 118 | SU1426 | (4-(3-amino-5-(2-aminopyridin-4-yl)-1H- | 29 | | 406 | |
| | | indazol-7-yl)phenyl)methanol | | | | |
| 119 | SU1430 | 5-(2-aminopyridin-4-yl)-7-(4- | 84 | | 2514 | |
| | | (dimethylamino)phenyl)-1H-indazol-3-amine | | | | |
| 120 | SU1420 | 4-(3-amino-5-(2-aminopyridin-4-yl)-1H- | 56 | | 1592 | |
| | | indazol-7-yl)benzamide | | | | |
| 121 | SU1427 | 4-(3-amino-5-(2-aminopyridin-4-yl)-1H- | 27 | | 1483 | |
| | | indazol-7-yl)benzenesulfonamide | | | | |
| 122 | SU1417 | 5-(2-aminopyridin-4-yl)-7-(4- | 52 | | 1257 | |
| | | (morpholinomethyl)phenyl)-1H-indazol-3- | | | | |
| | | amine | | | | |
| 123 | SU1643 | 5-(2-aminopyridin-4-yl)-7-(4-(tert- | 160 | | 1500 | |
| | | butyl)phenyl)-1H-indazol-3-amine | | | | |
| 124 | SU1428 | 5-(2-aminopyridin-4-yl)-7-(2-chlorophenyl)- | 224 | | 1760 | |
| | | 1H-indazol-3-amine | | | | |
| 125 | SU1425 | (2-(3-amino-5-(2-aminopyridin-4-yl)-1H- | 183 | | 796 | |
| | | indazol-7-yl)phenyl)methanol | | | | |
| 126 | SU1443 | 4-(3-amino-5-(2-aminopyridin-4-yl)-1H- | 571 | | 5029 | |
| | | indazol-7-yl)-3-methylbenzenesulfonamide | | | | |
| 127 | SU1431 | 5-(2-aminopyridin-4-yl)-7-(pyridin-3-yl)-1H- | 114 | | 1676 | |
| | | indazol-3-amine | | | | |
| 128 | SU1436 | 5-(2-aminopyridin-4-yl)-7-(pyridin-4-yl)-1H- | 35 | | 1300 | |
| | | indazol-3-amine | | | | |
| 129 | SU1438 | 5-(2-aminopyridin-4-yl)-7-(furan-3-yl)-1H- | 10 | | 511 | |
| | | indazol-3-amine | 5 | | 135 | |
| 130 | SU1440 | 5-(2-aminopyridin-4-yl)-7-(thiophen-3-yl)-1H- | 43 | | 612 | |
| | | indazol-3-amine | | | | |
| 131 | SU1442 | 5-(2-aminopyridin-4-yl)-7-(thiophen-2-yl)-1H- | 21 | | 224 | |
| | | indazol-3-amine | | | | |
| 132 | SU1446 | 5-(2-aminopyridin-4-yl)-7-(thiazol-5-yl)-1H- | 26 | | 316 | |
| | | indazol-3-amine | | | | |
| 133 | SU1441 | 5-(2-aminopyridin-4-yl)-7-(1H-pyrazol-5-yl)- | 46 | | 412 | |
| | | 1H-indazol-3-amine | | | | |
| 134 | SU1639 | 5-(2-aminopyridin-4-yl)-7-(3-methylbut-1-yn-1- | 3 | | 300 | |
| | | yl)-1H-indazol-3-amine | | | | |
| 135 | SU1670 | 5-(2-aminopyridin-4-yl)-7-(pent-1-yn-1-yl)-1H- | | | | |
| | | indazol-3-amine | | | | |
| 136 | SU1674 | 5-(2-aminopyridin-4-yl)-7- | | | | |
| | | (cyclopropylethynyl)-1H-indazol-3-amine | | | | |
| 137 | SU1433 | 5-(2-aminopyridin-4-yl)-7-(3,3-dimethylbut-1- | 53 | | 1467 | |
| | | yn-1-yl)-1H-indazol-3-amine | 11 | | 2295 | |
| 138 | SU1434 | 5-(2-aminopyridin-4-yl)-7-(phenylethynyl)-1H- | 81 | | 5029 | |
| | | indazol-3-amine | | | | |
| 139 | SU1435 | 4-(3-amino-5-(2-aminopyridin-4-yl)-1H- | 186 | | 4610 | |
| | | indazol-7-yl)but-3-yn-1-ol | | | | |
| 140 | SU1609 | 4-(3-amino-5-(2-aminopyridin-4-yl)-1H- | 281 | | 3000 | |
| | | indazol-7-yl)-2-methylbut-3-yn-2-ol | 39 | | 3088 | |
| 141 | SU1642 | 5-(2-aminopyridin-4-yl)-7-((3-methyloxetan-3- | 14 | | 380 | |
| | | yl)ethynyl)-1H-indazol-3-amine | | | | |
| 142 | SU1640 | 5-(2-aminopyridin-4-yl)-7-((tetrahydro-2H- | 20 | | 700 | |
| | | pyran-4-yl)ethynyl)-1H-indazol-3-amine | | | | |
| 143 | SU1661 | 5-(2-aminopyrimidin-4-yl)-7-(3,3-dimethylbut- | 517 | | | |
| | | 1-yn-1-yl)-1H-indazol-3-amine | | | | |
| 144 | SU1669 | 5-(2-aminopyridin-4-yl)-7-(3,3-dimethylbutyl)- | 15 | | 318 | |
| | | 1H-indazol-3-amine | | | | |
| 145 | SU1672 | 5-(2-aminopyridin-4-yl)-7-(2-cyclohexylethyl)- | | | | |
| | | 1H-indazol-3-amine | | | | |
| 146 | SU1675 | 5-(2-aminopyridin-4-yl)-7-(2-cyclopropylethyl)- | | | | |
| | | 1H-indazol-3-amine | | | | |
| 147 | SU1676 | 5-(2-aminopyridin-4-yl)-7-phenethyl-1H- | | | | |
| | | indazol-3-amine | | | | |
| 148 | SU1636 | 5-(2-amino-5-fluoropyridin-4-yl)-7-(3,3- | 23 | | 2330 | |
| | | dimethylbut-1-yn-1-yl)-1H-indazol-3-amine | | | | |
| 149 | SU1638 | 5-(2-amino-3-fluoropyridin-4-yl)-7-(3,3- | 8 | | 220 | |
| | | dimethylbut-1-yn-1-yl)-1H-indazol-3-amine | | | | |
| 150 | SU1666 | 5-(2-amino-6-fluoropyridin-4-yl)-7-(3,3- | 573 | | | |
| | | dimethylbut-1-yn-1-yl)-1H-indazol-3-amine | | | | |
| 151 | SU1660 | 4-(3-amino-7-(3,3-dimethylbut-1-yn-1-yl)-1H- | 931 | | | |
| | | indazol-5-yl)pyridine-2,6-diamine | | | | |
| 153 | SU1645 | 5-(2-(cyclopropylamino)pyridine-4-yl)-7-(3,3- | 4 | | 100 | |
| | | dimethylbut-1-yn-1-yl)-1H-indazol-3-amine | | | | |
| 154 | SU1691 | 5-(2-(cyclobutylamino)pyridin-4-yl)-7-(3,3- | 120 | | 1690 | |
| | | dimethylbut-1-yn-1-yl)-1H-indazol-3-amine | | | | |
| 155 | SU1699 | 7-(3,3-dimethylbut-1-yn-1-yl)-5-(2-(oxetan-3- | 15 | | 784 | |
| | | ylamino)pyridin-4-yl)-1H-indazol-3-amine | | | | |
| 156 | SU1646 | 5-(2-(cyclopentylamino)pyridine-4-yl)-7-(3,3- | | | | |
| | | dimethylbut-1-yn-1-yl)-1H-indazol-3-amine | | | | |
| 157 | SU1693 | 5-(2-((cyclopropylmethyl)amino)pyridin-4-yl)- | 141 | | 2800 | |
| | | 7-(3,3-dimethylbut-1-yn-1-yl)-1H-indazol-3- | | | | |
| | | amine | | | | |
| 158 | SU1701 | 7-(3,3-dimethylbut-1-yn-1-yl)-5-(2-((2,2,2- | 195 | | 16300 | |
| | | trifluoroethyl)amino)pyridin-4-yl)-1H-indazol-3- | | | | |
| | | amine | | | | |
| 159 | SU1702 | 3-((4-(3-amino-7-(3,3-dimethylbut-1-yn-1-yl)- | 22 | | 1960 | |
| | | 1H-indazol-5-yl)pyridin-2- | | | | |
| | | yl)amino)propanenitrile | | | | |
| 160 | SU1697 | 2-((4-(3-amino-7-(3,3-dimethylbut-1-yn-1-yl)- | 8 | | 1340 | |
| | | 1H-indazol-5-yl)pyridin-2-yl)amino)ethan-1-ol | | | | |
| 161 | SU1698 | N1-(4-(3-amino-7-(3,3-dimethylbut-1-yn-1-yl)- | 128 | | 7610 | |
| | | 1H-indazol-5-yl)pyridin-2-yl)ethane-1,2- | | | | |
| | | diamine | | | | |
| 162 | SU1692 | 7-(3,3-dimethylbut-1-yn-1-yl)-5-(2-((2- | 35 | | 1110 | |
| | | methoxyethyl)amino)pyridin-4-yl)-1H-indazol- | | | | |
| | | 3-amine | | | | |
| 163 | SU1641 | 7-(3,3-dimethylbut-1-yn-1-yl)-5-(2-((3- | >3000 | | >30000 | |
| | | methoxypropyl)amino)pyridine-4-yl)-1H- | | | | |
| | | indazol-3-amine | | | | |
| 164 | SU1644 | N-(4-(3-amino-7-(3,3-dimethylbut-1-yn-1-yl)- | 15 | | 350 | |
| | | 1H-indazol-5-yl)pyridin-2-yl)acetamide | | | | |
| 165 | SU1682 | N-(4-(3-amino-7-(3,3-dimethylbut-1-yn-1-yl)- | 7 | | 780 | |
| | | 1H-indazol-5-yl)pyridin-2-yl)propionamide | | | | |
| 166 | SU1708 | N-(4-(3-amino-7-(3,3-dimethylbut-1-yn-1-yl)- | | | | |
| | | 1H-indazol-5-yl)pyridin-2-yl)-3,3,3- | | | | |
| | | trifluoropropanamide | | | | |
| 167 | SU1688 | N-(4-(3-amino-7-(3,3-dimethylbut-1-yn-1-yl)- | 15 | | 1110 | |
| | | 1H-indazol-5-yl)pyridin-2- | | | | |
| | | yl)cyclopropanecarboxamide | | | | |
| 168 | SU1648 | N-(4-(3-amino-7-(3,3-dimethylbut-1-yn-1-yl)- | 273 | | 2580 | |
| | | 1H-indazol-5-yl)pyridin-2-yl)isobutyramide | | | | |
| 169 | SU1649 | N-(4-(3-amino-7-(3,3-dimethylbut-1-yn-1-yl)- | | | | |
| | | 1H-indazol-5-yl)pyridin-2-yl)pivalamide | | | | |
| 170 | SU1696 | N-(4-(3-amino-7-(3,3-dimethylbut-1-yn-1-yl)- | 26 | | 1550 | |
| | | 1H-indazol-5-yl)pyridin-2-yl)-2- | | | | |
| | | cyclopropylacetamide | | | | |
| 171 | SU1647 | N-(4-(3-amino-7-(3,3-dimethylbut-1-yn-1-yl)- | 122 | | 5680 | |
| | | 1H-indazol-5-yl)pyridine-2-yl)-3- | | | | |
| | | methylbutanamide | | | | |
| 172 | SU1695 | N-(4-(3-amino-7-(3,3-dimethylbut-1-yn-1-yl)- | 89 | | 3330 | |
| | | 1H-indazol-5-yl)pyridin-2- | | | | |
| | | yl)cyclobutanecarboxamide | | | | |
| 173 | SU1652 | N-(4-(3-amino-7-(3,3-dimethylbut-1-yn-1-yl)- | 298 | | 23800 | |
| | | 1H-indazol-5-yl)pyridin-2- | | | | |
| | | yl)cyclopentanecarboxamide | | | | |
| 174 | SU1689 | N-(4-(3-amino-7-(3,3-dimethylbut-1-yn-1-yl)- | 20 | | 611 | |
| | | 1H-indazol-5-yl)pyridin-2-yl)-2- | | | | |
| | | hydroxyacetamide | | | | |
| 175 | SU1690 | N-(4-(3-amino-7-(3,3-dimethylbut-1-yn-1-yl)- | 24 | | 1110 | |
| | | 1H-indazol-5-yl)pyridin-2-yl)-2- | | | | |
| | | methoxyacetamide | | | | |
| 176 | SU1654 | 5-(2-(cyclopropylamino)pyridine-4-yl)-7-((3- | 19 | | | |
| | | methyloxetan-3-yl)ethynyl)-1H-indazol-3- | | | | |
| | | amine | | | | |
| 177 | SU1655 | N-(4-(3-amino-7-((3-methyloxetan-3- | | | | |
| | | yl)ethynyl)-1H-indazol-5-yl)pyridin-2- | | | | |
| | | yl)acetamide | | | | |
| 178 | SU1658 | N-(4-(3-amino-7-(phenylethynyl)-1H-indazol- | 22 | | | |
| | | 5-yl)pyridin-2-yl)acetamide | | | | |
| 179 | SU1704 | methyl (4-(3-amino-7-(cyclopropylethynyl)-1H- | 10 | | 962 | |
| | | indazol-5-yl)pyridin-2-yl)carbamate | | | | |
| 180 | SU1703 | methyl (4-(3-amino-7-(3-hydroxy-3-methylbut- | 5 | | 826 | |
| | | 1-yn-1-yl)-1H-indazol-5-yl)pyridin-2- | | | | |
| | | yl)carbamate | | | | |
| 181 | SU1705 | methyl (4-(3-amino-7-(3-amino-3-methylbut-1- | 20 | | 515 | |
| | | yn-1-yl)-1H-indazol-5-yl)pyridin-2- | | | | |
| | | yl)carbamate | | | | |
| 182 | SU1707 | methyl (4-(3-amino-7-(3-methoxy-3- | 34 | | 363 | |
| | | methylbut-1-yn-1-yl)-1H-indazol-5-yl)pyridin- | | | | |
| | | 2-yl)carbamate | | | | |
| 183 | SU1709 | methyl (4-(3-amino-7-(3-morpholinoprop-1-yn- | 47 | | 721 | |
| | | 1-yl)-1H-indazol-5-yl)pyridin-2-yl)carbamate | | | | |
| 184 | SU1711 | Methyl (4-(3-amino-7-(4-morpholinobut-1-yn-1-yl)- | | | | |
| | | 1H-indazol-5-yl)pyridin-2-yl)carbamate | | | | |
| 185 | SU1694 | 1-(4-(3-amino-7-(3,3-dimethylbut-1-yn-1-yl)- | | | | |
| | | 1H-indazol-5-yl)pyridin-2-yl)urea | | | | |
| 186 | SU1686 | 1-(4-(3-amino-7-(3,3-dimethylbut-1-yn-1-yl)- | 34 | | 3490 | |
| | | 1H-indazol-5-yl)pyridin-2-yl)-3-methylurea | | | | |
| 187 | SU1650 | 1-(4-(3-amino-7-(3,3-dimethylbut-1-yn-1-yl)- | 438 | | 8100 | |
| | | 1H-indazol-5-yl)pyridin-2-yl)-3-ethylurea | | | | |
| 188 | SU1651 | 1-(4-(3-amino-7-(3,3-dimethylbut-1-yn-1-yl)- | | | | |
| | | 1H-indazol-5-yl)pyridin-2-yl)-3-propylurea | | | | |
| 189 | SU1653 | 1-(4-(3-amino-7-(3,3-dimethylbut-1-yn-1-yl)- | 113 | | 19300 | |
| | | 1H-indazol-5-yl)pyridine-2-yl)-3-phenylurea | | | | |
| 190 | SU1680 | methyl (4-(3-amino-7-(3,3-dimethylbut-1-yn-1-yl)- | 12 | | 1170 | |
| | | 1H-indazol-5-yl)pyridin-2-yl)carbamate | | | | |
| 191 | SU1683 | ethyl (4-(3-amino-7-(3,3-dimethylbut-1-yn-1-yl)- | 20 | | 1590 | |
| | | 1H-indazol-5-yl)pyridin-2-yl)carbamate | | | | |
| 192 | SU1668 | tert-butyl (4-(3-amino-7-(3,3-dimethylbut-1-yn-1- | | | | |
| | | yl)-1H-indazol-5-yl)pyridin-2-yl)carbamate | | | | |
| 193 | SU1656 | (4-(3-amino-7-(3,3-dimethylbut-1-yn-1-yl)-1H- | 5,520 | | | |
| | | indazol-5-yl)pyridin-2-yl)sulfamic acid | | | | |
| 194 | SU1700 | N-(4-(3-amino-7-(3,3-dimethylbut-1-yn-1-yl)- | 373 | | 4900 | |
| | | 1H-indazol-5-yl)pyridin-2- | | | | |
| | | yl)methanesulfonamide | | | | |
| 195 | SU1667 | N-(4-(3-amino-7-(3,3-dimethylbut-1-yn-1-yl)- | 21,800 | | | |
| | | 1H-indazol-5-yl)-6-fluoropyridin-2- | | | | |
| | | yl)acetamide | | | | |
| 196 | SU1663 | N-(4-(3-amino-7-phenyl-1H-indazol-5- | . | | | |
| | | yl)pyridin-2-yl)acetamide | | | | |
| 197 | SU1662 | N-(4-(3-amino-7-(pyridin-4-yl)-1H-indazol-5- | 25 | | 70 | |
| | | yl)pyridin-2-yl)acetamide | | | | |
| 199 | SU1518 | 7-(furan-3-yl)-5-(1H-pyrrolo[2,3-b]pyridin-4-yl)- | 6 | | 45 | |
| | | 1H-indazol-3-amine | | | | |
| 200 | SU1492 | 7-ethynyl-5-(1H-pyrrolo[2,3-b]pyridin-4-yl)-1H- | 26 | | 137 | |
| | | indazol-3-amine | 3.3 | | 158 | |
| 201 | SU1399 | 7-(3,3-dimethylbut-1-yn-1-yl)-5-(1H- | 62 | | 1391 | |
| | | pyrrolo[2,3-b]pyridin-4-yl)-1H-indazol-3-amine | 11 | | 1205 | |
| 202 | SU1525 | 7-(cyclopropylethynyl)-5-(1H-pyrrolo[2,3- | 13 | | 181 | |
| | | b]pyridin-4-yl)-1H-indazol-3-amine | 2 | | 470 | |
| 203 | SU1526 | 7-(cyclopentylethynyl)-5-(1H-pyrrolo[2,3- | 26 | | 452 | |
| | | b]pyridin-4-yl)-1H-indazol-3-amine | 16 | | 1530 | |
| 204 | SU1506 | 7-(cyclohexylethynyl)-5-(1H-pyrrolo[2,3- | 169 | | 1178 | |
| | | b]pyridin-4-yl)-1H-indazol-3-amine | 198 | | 13500 | |
| 205 | SU1519 | 3-(3-amino-5-(1H-pyrrolo[2,3-b]pyridin-4-yl)- | 366 | | 3100 | |
| | | 1H-indazol-7-yl)prop-2-yn-1-ol | | | | |
| 206 | SU1505 | 4-(3-amino-5-(1H-pyrrolo[2,3-b]pyridin-4-yl)- | 81 | | 1519 | |
| | | 1H-indazol-7-yl)-2-methylbut-3-yn-2-ol | 9 | | 294 | |
| 207 | SU1521 | 1-((3-amino-5-(1H-pyrrolo[2,3-b]pyridin-4-yl)- | 82 | | 1467 | |
| | | 1H-indazol-7-yl)ethynyl)cyclopentan-1-ol | | | | |
| 208 | SU1522 | 1-((3-amino-5-(1H-pyrrolo[2,3-b]pyridin-4-yl)- | 76 | | 528 | |
| | | 1H-indazol-7-yl)ethynyl)cyclohexan-1-ol | | | | |
| 209 | SU1517 | 1-((3-amino-5-(1H-pyrrolo[2,3-b]pyridin-4-yl)- | 153 | | 2334 | |
| | | 1H-indazol-7-yl)ethynyl)cycloheptan-1-ol | | | | |
| 210 | SU1432 | 7-(5-morpholinopent-1-yn-1-yl)-5-(1H- | 23 | | 3100 | |
| | | pyrrolo[2,3-b]pyridin-4-yl)-1H-indazol-3-amine | 27 | | 1030 | |
| 211 | SU1479 | 7-(4-(piperidin-1-yl)but-1-yn-1-yl)-5-(1H- | 19 | | 356 | |
| | | pyrrolo[2,3-b]pyridin-4-yl)-1H-indazol-3-amine | | | | |
| 212 | SU1419 | 7-(5-(piperidin-1-yl)pent-1-yn-1-yl)-5-(1H- | 30 | | 2374 | |
| | | pyrrolo[2,3-b]pyridin-4-yl)-1H-indazol-3-amine | 12 | | 1135 | |
| 213 | SU1472 | 7-(6-(piperidin-1-yl)hex-1-yn-1-yl)-5-(1H- | 40 | | 2514 | |
| | | pyrrolo[2,3-b]pyridin-4-yl)-1H-indazol-3-amine | | | | |
| 214 | SU1476 | 6-(3-amino-5-(1H-pyrrolo[2,3-b]pyridin-4-yl)- | 21 | | 268 | |
| | | 1H-indazol-7-yl)hex-5-ynoic acid | | | | |
| 215 | SU1475 | 7-(3-amino-5-(1H-pyrrolo[2,3-b]pyridin-4-yl)- | 38 | | 256 | |
| | | 1H-indazol-7-yl)hept-6-ynoic acid | | | | |
| 216 | SU1478 | 7-(4-phenoxybut-1-yn-1-yl)-5-(1H-pyrrolo[2,3- | 16 | | 81 | |
| | | b]pyridin-4-yl)-1H-indazol-3-amine | | | | |
| 217 | SU1473 | 7-(6-phenoxyhex-1-yn-1-yl)-5-(1H-pyrrolo[2,3- | 243 | | 1260 | |
| | | b]pyridin-4-yl)-1H-indazol-3-amine | | | | |
| 218 | SU1544 | 5-(2-cyclopropyl-1H-pyrrolo[2,3-b]pyridin-4- | 5 | | 63 | |
| | | yl)-1H-indazol-3-amine | 1 | | 17 | |
| 219 | SU1483 | 5-(2-cyclohexyl-1H-pyrrolo[2,3-b]pyridin-4-yl)- | 42 | | 461 | |
| | | 1H-indazol-3-amine | 14 | | 280 | |
| 220 | SU1527 | 5-(2-neopentyl-1H-pyrrolo[2,3-b]pyridin-4-yl)- | 58 | | 838 | |
| | | 1H-indazol-3-amine | 2 | | 483 | |
| 221 | SU1480 | 5-(2-(cyclohexylmethyl)-1H-pyrrolo[2,3- | 38 | | 461 | |
| | | b]pyridin-4-yl)-1H-indazol-3-amine | 8 | | 655 | |
| 222 | SU1545 | 5-(2-(2-cyclohexylethyl)-1H-pyrrolo[2,3- | 19 | | 134 | |
| | | b]pyridin-4-yl)-1H-indazol-3-amine | 26 | | 268 | |
| 223 | SU1603 | 5-(2-benzyl-1H-pyrrolo[2,3-b]pyridin-4-yl)-1H- | 1 | | 111 | |
| | | indazol-3-amine | | | | |
| 224 | SU1396 | (4-(3-amino-1H-indazol-5-yl)-1H-pyrrolo[2,3- | 5 | | 503 | |
| | | b]pyridin-2-yl)methanol | 1 | | 28 | |
| 225 | SU1290 | 2-(4-(3-amino-1H-indazol-5-yl)-1H- | 26 | | 419 | |
| | | pyrrolo[2,3-b]pyridin-2-yl)propan-2-ol | | | | |
| 226 | SU1391 | 3-(4-(3-amino-1H-indazol-5-yl)-1H- | 95 | | 922 | |
| | | pyrrolo[2,3-b]pyridin-2-yl)pentan-3-ol | | | | |
| 227 | SU1535 | 5-(2-(tert-butoxymethyl)-1H-pyrrolo[2,3- | 29 | | 754 | |
| | | b]pyridin-4-yl)-1H-indazol-3-amine | 2 | | 388 | |
| 228 | SU1529 | 5-(2-(tetrahydro-2H-pyran-4-yl)-1H- | 12 | | 97 | |
| | | pyrrolo[2,3-b]pyridin-4-yl)-1H-indazol-3-amine | 1 | | 32 | |
| 229 | SU1530 | 5-(2-(tetrahydro-2H-pyran-2-yl)-1H- | 27 | | 272 | |
| | | pyrrolo[2,3-b]pyridin-4-yl)-1H-indazol-3-amine | 1 | | 225 | |
| 230 | SU1528 | 5-(2-((tetrahydro-2H-pyran-4-yl)methyl)-1H- | 11 | | 260 | |
| | | pyrrolo[2,3-b]pyridin-4-yl)-1H-indazol-3-amine | 1 | | 60 | |
| 231 | SU1289 | methyl 4-(3-amino-1H-indazol-5-yl)-1H- | 9 | | 419 | |
| | | pyrrolo[2,3-b]pyridine-2-carboxylate | | | | |
| 232 | SU1279 | ethyl 4-(3-amino-1H-indazol-5-yl)-1H- | 10 | | 1257 | |
| | | pyrrolo[2,3-b]pyridine-2-carboxylate | | | | |
| 233 | SU1687 | 4-(3-amino-1H-indazol-5-yl)-1H-pyrrolo[2,3- | | | | |
| | | b]pyridine-2-carboxamide | | | | |
| 234 | SU1300 | (4-(3-amino-1H-indazol-5-yl)-1H-pyrrolo[2,3- | 19 | | 272 | |
| | | b]pyridin-2-yl)(pyrrolidin-1-yl)methanone | 32 | | 790 | |
| 235 | SU1313 | 4-(3-amino-1H-indazol-5-yl)-N-cyclopentyl- | 6 | | 70 | |
| | | 1H-pyrrolo[2,3-b]pyridine-2-carboxamide | | | | |
| 236 | SU1309 | 4-(3-amino-1H-indazol-5-yl)-N-cyclohexyl-1H- | 10 | | 79 | |
| | | pyrrolo[2,3-b]pyridine-2-carboxamide | 2 | | 20 | |
| 237 | SU1292 | 4-(3-amino-1H-indazol-5-yl)-N-isopentyl-1H- | 3 | | 130 | |
| | | pyrrolo[2,3-b]pyridine-2-carboxamide | | | | |
| 238 | SU1320 | 4-(3-amino-1H-indazol-5-yl)-N-phenethyl-1H- | 23 | | 90 | |
| | | pyrrolo[2,3-b]pyridine-2-carboxamide | | | | |
| 239 | SU1315 | 4-(3-amino-1H-indazol-5-yl)-N-(3- | 5 | | 60 | |
| | | phenylpropyl)-1H-pyrrolo[2,3-b]pyridine-2- | | | | |
| | | carboxamide | | | | |
| 240 | SU1298 | 4-(3-amino-1H-indazol-5-yl)-N-(2- | 5 | | 30 | |
| | | methoxyethyl)-1H-pyrrolo[2,3-b]pyridine-2- | | | | |
| | | carboxamide | | | | |
| 241 | SU1326 | 4-(3-amino-1H-indazol-5-yl)-N-(2-aminoethyl)- | 4 | | 122 | |
| | | 1H-pyrrolo[2,3-b]pyridine-2-carboxamide | | | | |
| 242 | SU1299 | 4-(3-amino-1H-indazol-5-yl)-N-(2- | 8 | | 377 | |
| | | (dimethylamino)ethyl)-1H-pyrrolo[2,3- | | | | |
| | | b]pyridine-2-carboxamide | | | | |
| 243 | SU1306 | (4-(3-amino-1H-indazol-5-yl)-1H-pyrrolo[2,3- | 72 | | 1031 | |
| | | b]pyridin-2-yl)(4-methylpiperazin-1- | 13 | | 630 | |
| | | yl)methanone | | | | |
| 244 | SU1314 | 4-(3-amino-1H-indazol-5-yl)-N-(2-(piperidin-1- | 8 | | 109 | |
| | | yl)ethyl)-1H-pyrrolo[2,3-b]pyridine-2- | | | | |
| | | carboxamide | | | | |
| 245 | SU1321 | 4-(3-amino-1H-indazol-5-yl)-N-(2- | 29 | | 193 | |
| | | (butyl(ethyl)amino)ethyl)-1H-pyrrolo[2,3- | | | | |
| | | b]pyridine-2-carboxamide | | | | |
| 246 | SU1327 | 4-(3-amino-1H-indazol-5-yl)-N-(2- | 9 | | 104 | |
| | | (diisopropylamino)ethyl)-1H-pyrrolo[2,3- | | | | |
| | | blpyridine-2-carboxamide | | | | |
| 247 | SU1305 | 4-(3-amino-1H-indazol-5-yl)-N-(3- | 8 | | 46 | |
| | | (dimethylamino)propyl)-1H-pyrrolo[2,3- | | | | |
| | | b]pyridine-2-carboxamide | | | | |
| 248 | SU1453 | 5-(2-((tert-butylamino)methyl)-1H-pyrrolo[2,3- | 202 | | 4023 | |
| | | b]pyridine-4-yl)-1H-indazol-3-amine | 34 | | 1430 | |
| 249 | SU1458 | 5-(2-((isopentylamino)methyl)-1H-pyrrolo[2,3- | 56 | | 1467 | |
| | | b]pyridine-4-yl)-1H-indazol-3-amine | | | | |
| 250 | SU1514 | 5-(2-(piperidin-2-yl)-1H-pyrrolo[2,3-b]pyridine- | 380 | | >10000 | |
| | | 4-yl)-1H-indazol-3-amine | 41 | | 810 | |
| 251 | SU1454 | 5-(2-((cyclohexylamino)methyl)-1H- | 74 | | 1299 | |
| | | pyrrolo[2,3-b]pyridine-4-yl)-1H-indazol-3- | 6 | | 405 | |
| | | amine | | | | |
| 252 | SU1469 | 5-(2-((phenylamino)methyl)-1H-pyrrolo[2,3- | 6 | | 59 | |
| | | b]pyridine-4-yl)-1H-indazol-3-amine | | | | |
| 253 | SU1606 | 5-(2-(((2-(benzyloxy)phenyl)amino)methyl)- | 5 | | 330 | |
| | | 1H-pyrrolo[2,3-b]pyridine-4-yl)-1H-indazol-3- | | | | |
| | | amine | | | | |
| 254 | SU1459 | 5-(2-(((2-methoxyethyl)amino)methyl)-1H- | 18 | | 389 | |
| | | pyrrolo[2,3-b]pyridine-4-yl)-1H-indazol-3- | | | | |
| | | amine | | | | |
| 255 | SU1462 | N¹-((4-(3-amino-1H-indazol-5-yl)-1H- | 30 | | 1634 | |
| | | pyrrolo[2,3-b]pyridin-2-yl)methyl)-N²,N²- | | | | |
| | | dimethylethane-1,2-diamine | | | | |
| 256 | SU1464 | 5-(2-(((3-methoxypropyl)amino)methyl)-1H- | 46 | | 2556 | |
| | | pyrrolo[2,3-b]pyridine-4-yl)-1H-indazol-3- | | | | |
| | | amine | | | | |
| 257 | SU1463 | 5-(2-(((3-isopropoxypropyl)amino)methyl)-1H- | 40 | | 2221 | |
| | | pyrrolo[2,3-b]pyridine-4-yl)-1H-indazol-3- | | | | |
| | | amine | | | | |
| 258 | SU1466 | N1-((4-(3-amino-1H-indazol-5-yl)-1H- | 16 | | 1131 | |
| | | pyrrolo[2,3-b]pyridin-2-yl)methyl)-N³,N³- | 10 | | 245 | |
| | | dimethylpropane-1,3-diamine | | | | |
| 259 | SU1455 | 5-(2-((isopropyl(methyl)amino)methyl)-1H- | 110 | | 2137 | |
| | | pyrrolo[2,3-b]pyridine-4-yl)-1H-indazol-3- | 32 | | 790 | |
| | | amine | | | | |
| 260 | SU1460 | 5-(2-(piperidin-1-ylmethyl)-1H-pyrrolo[2,3- | 91 | | 1718 | |
| | | b]pyridine-4-yl)-1H-indazol-3-amine | 27 | | 454 | |
| 261 | SU1464 | 5-(2-((4,4-difluoropiperidin-1-yl)methyl)-1H- | 49 | | 2389 | |
| | | pyrrolo[2,3-b]pyridine-4-yl)-1H-indazol-3- | 11 | | 286 | |
| | | amine | | | | |
| 262 | SU1456 | 5-(2-(morpholinomethyl)-1H-pyrrolo[2,3- | 22 | | 277 | |
| | | b]pyridine-4-yl)-1H-indazol-3-amine | 2 | | 269 | |
| 263 | SU1461 | 5-(2-((4-methylpiperazin-1-yl)methyl)-1H- | 62 | | 2430 | |
| | | pyrrolo[2,3-b]pyridine-4-yl)-1H-indazol-3- | 14 | | 300 | |
| | | amine | | | | |
| 264 | SU1620 | 5-(2-((4-(tert-butyl)piperazin-1-yl)methyl)-1H- | 5 | | 285 | |
| | | pyrrolo[2,3-b]pyridin-4-yl)-1H-indazol-3-amine | | | | |
| 265 | SU1467 | 5-(2-(azepan-1-ylmethyl)-1H-pyrrolo[2,3- | 91 | | 2514 | |
| | | b]pyridine-4-yl)-1H-indazol-3-amine | 13 | | 350 | |
| 266 | SU1468 | 5-(2-((4-methyl-1,4-diazepan-1-yl)methyl)-1H- | 46 | | 1802 | |
| | | pyrrolo[2,3-b]pyridin-4-yl)-1H-indazol-3-amine | 11 | | 380 | |
| 267 | SU1513 | 5-(2-(2-(piperidin-1-yl)ethyl)-1H-pyrrolo[2,3- | 53 | | 1383 | |
| | | b]pyridine-4-yl)-1H-indazol-3-amine | 8 | | 235 | |
| 268 | SU1512 | 5-(2-(2-morpholinoethyl)-1H-pyrrolo[2,3- | 11 | | 251 | |
| | | b]pyridin-4-yl)-1H-indazol-3-amine | 1 | | 138 | |
| 269 | SU1489 | 5-(2-(3-(piperidin-1-yl)propyl)-1H-pyrrolo[2,3- | 11 | | 507 | |
| | | b]pyridin-4-yl)-1H-indazol-3-amine | 4 | | 46 | |
| 270 | SU1491 | 5-(2-(3-(cyclohexylamino)propyl)-1H- | 4 | | 65 | |
| | | pyrrolo[2,3-b]pyridin-4-yl)-1H-indazol-3-amine | | | | |
| 271 | SU1511 | 5-(2-(3-morpholinopropyl)-1H-pyrrolo[2,3- | 11 | | 126 | |
| | | b]pyridin-4-yl)-1H-indazol-3-amine | 1 | | 111 | |
| 272 | SU1600 | 5-(2-(piperidin-4-ylmethyl)-1H-pyrrolo[2,3- | 5 | | 50 | |
| | | b]pyridin-4-yl)-1H-indazol-3-amine | 1 | | 53 | |
| 273 | SU1604 | 5-(2-((1-benzylpiperidin-4-yl)methyl)-1H- | 5 | | 200 | |
| | | pyrrolo[2,3-b]pyridin-4-yl)-1H-indazol-3-amine | | | | |
| 274 | SU1418 | 4-(3-amino-7-chloro-1H-indazol-5-yl)-N-(2- | 8 | | 126 | |
| | | (piperidin-1-yl)ethyl)-1H-pyrrolo[2,3- | | | | |
| | | b]pyridine-2-carboxamide | | | | |
| 275 | SU1624 | 7-chloro-5-(2-(cyclohexylmethyl)-1H- | 37 | | 376 | |
| | | pyrrolo[2,3-b]pyridin-4-yl)-1H-indazol-3-amine | | | | |
| 276 | SU1633 | 7-chloro-5-(2-(morpholinomethyl)-1H- | 4 | | 70 | |
| | | pyrrolo[2,3-b]pyridin-4-yl)-1H-indazol-3-amine | | | | |
| 277 | SU1625 | 7-chloro-5-(2-(2-morpholinoethyl)-1H- | 1 | | 51 | |
| | | pyrrolo[2,3-b]pyridin-4-yl)-1H-indazol-3-amine | | | | |
| 278 | SU1623 | 5-(2-(cyclohexylmethyl)-1H-pyrrolo[2,3- | 380 | | >10,000 | |
| | | b]pyridin-4-yl)-7-(3,3-dimethylbut-1-yn-1-yl)- | | | | |
| | | 1H-indazol-3-amine | | | | |
| 279 | SU1632 | 7-(3,3-dimethylbut-1-yn-1-yl)-5-(2- | 13 | | 370 | |
| | | (morpholinomethyl)-1H-pyrrolo[2,3-b]pyridin- | | | | |
| | | 4-yl)-1H-indazol-3-amine | | | | |
| 280 | SU1622 | 7-(3,3-dimethylbut-1-yn-1-yl)-5-(2-(2- | 30 | | 360 | |
| | | morpholinoethyl)-1H-pyrrolo[2,3-b]pyridin-4- | | | | |
| | | yl)-1H-indazol-3-amine | | | | |
| 281 | SU1626 | 5-(2-(2-morpholinoethyl)-1H-pyrrolo[2,3- | 47 | | 331 | |
| | | b]pyridin-4-yl)-7-(phenylethynyl)-1H-indazol-3- | | | | |
| | | amine | | | | |
| 282 | SU1733 | N-(4-(3-Amino-7-(3,3-dimethylbut-1-yn-1-yl)- | 19 | | 594 | |
| | | 1H-indazol-5-yl)pyridin-2-yl)oxazol-2-amine | | | | |
| 283 | SU1717 | 7-(3,3-Dimethylbut-1-yn-1-yl)-5-(2-((3,3,3- | 215 | | >10000 | |
| | | trifluoropropyl)amino)pyridin-4-yl)-1H-indazol- | | | | |
| | | 3-amine | | | | |
| 284 | SU1719 | 7-(Cyclopropylethynyl)-5-(2-(oxetan-3- | 12 | | 386 | |
| | | ylamino)pyridin-4-yl)-1H-indazol-3-amine | | | | |
| 285 | SU1730 | Methyl (4-(3-amino-7-((tetrahydro-2H-pyran- | 37 | | 274 | |
| | | 4-yl)ethynyl)-1H-indazol-5-yl)pyridin-2- | | | | |
| | | yl)carbamate | | | | |
| 286 | SU1736 | Methyl (4-(3-amino-7-((3-methyloxetan-3- | | | | |
| | | yl)ethynyl)-1H-indazol-5-yl)pyridin-2- | | | | |
| | | yl)carbamate | | | | |
| 287 | SU1713 | 4-(3-Amino-5-(2-(oxetan-3-ylamino)pyridin-4- | 93 | | 2050 | |
| | | yl)-1H-indazol-7-yl)-2-methylbut-3-yn-2-ol | | | | |
| 288 | SU1728 | 5-(2-(Oxetan-3-ylamino)pyridin-4-yl)-7- | 24 | | 500 | |
| | | ((tetrahydro-2H-pyran-4-yl)ethynyl)-1H- | | | | |
| | | indazol-3-amine | | | | |
| 289 | SU1729 | N-(4-(3-Amino-7-(cyclopropylethynyl)-1H- | 42 | | 570 | |
| | | indazol-5-yl)pyridin-2- | | | | |
| | | yl)cyclopropanecarboxamide | | | | |
| 290 | SU1714 | N-(4-(3-Amino-7-(3-hydroxy-3-methylbut-1- | 55 | | 242 | |
| | | yn-1-yl)-1H-indazol-5-yl)pyridin-2- | | | | |
| | | yl)cyclopropanecarboxamide | | | | |
| 291 | SU1735 | Methyl (4-(3-amino-7-(5-morpholinopent-1-yn- | | | | |
| | | 1-yl)-1H-indazol-5-yl)pyridin-2-yl)carbamate | | | | |
| 292 | SU1716 | N-(4-(3-Amino-7-(3-hydroxy-3-methylbutyl)- | 9 | | 44 | |
| | | 1H-indazol-5-yl)pyridin-2- | | | | |
| | | yl)cyclopropanecarboxamide | | | | |
| 293 | SU1732 | Methyl (4-(3-amino-7-(3,3-dimethylbutyl)-1H- | 19 | | 278 | |
| | | indazol-5-yl)pyridin-2-yl)carbamate | | | | |
| 294 | SU1721 | 5-(2-Cyclopropyl-1H-pyrrolo[2,3-b]pyridin-4- | 57 | | 322 | |
| | | yl)-7-(3,3-dimethylbut-1-yn-1-yl)-1H-indazol-3- | | | | |
| | | amine | | | | |
| 295 | SU1725 | 5-(2-Cyclopentyl-1H-pyrrolo[2,3-b]pyridin-4- | 545 | | 1160 | |
| | | yl)-7-(3,3-dimethylbut-1-yn-1-yl)-1H-indazol-3- | | | | |
| | | amine | | | | |
| 296 | SU1731 | 5-(2-(tert-Butyl)-1H-pyrrolo[2,3-b]pyridin-4-yl)- | 449 | | 1080 | |
| | | 7-(3,3-dimethylbut-1-yn-1-yl)-1H-indazol-3- | | | | |
| | | amine | | | | |
| 297 | SU1727 | (4-(3-Amino-7-(3,3-dimethylbut-1-yn-1-yl)-1H- | 2 | | 268 | |
| | | indazol-5-yl)-1H-pyrrolo[2,3-b]pyridin-2- | | | | |
| | | yl)methanol | | | | |
| 298 | SU1723 | 2-(4-(3-Amino-7-(3,3-dimethylbut-1-yn-1-yl)- | 16 | | 540 | |
| | | 1H-indazol-5-yl)-1H-pyrrolo[2,3-b]pyridin-2- | | | | |
| | | yl)propan-2-ol | | | | |
| 299 | SU1720 | Methyl 4-(3-amino-7-(3,3-dimethylbut-1-yn-1- | 10 | | >370 | |
| | | yl)-1H-indazol-5-yl)-1H-pyrrolo[2,3-b]pyridine- | | | | |
| | | 2-carboxylate | | | | |
| 300 | SU1734 | 5-(2-(Difluoromethyl)-3H-imidazo[4,5- | 13 | | 1340 | |
| | | b]pyridin-7-yl)-7-(3,3-dimethylbut-1-yn-1-yl)- | | | | |
| | | 1H-indazol-3-amine | | | | |
| 301 | SU1726 | 5-(2-Cyclobutyl-3H-imidazo[4,5-b]pyridin-7- | 82 | | 1240 | |
| | | yl)-7-(3,3-dimethylbut-1-yn-1-yl)-1H-indazol-3- | | | | |
| | | amine | | | | |
| 302 | SU1738 | 7-(3,3-dimethylbut-1-yn-1-yl)-5-(2-((1-methyl- | 62 | nM | 148 | nM |
| | | 1H-pyrazol-3-yl)amino)pyridin-4-yl)-1H- | | | | |
| | | indazol-3-amine | | | | |
| 303 | SU1739 | 7-(3,3-dimethylbut-1-yn-1-yl)-5-(2-((1-methyl- | 36 | nM | 68 | nM |
| | | 1H-pyrazol-4-yl)amino)pyridin-4-yl)-1H- | | | | |
| | | indazol-3-amine | | | | |
| 304 | SU1740 | methyl (4-(3-amino-7-((1- | 64 | nM | 275 | nM |
| | | hydroxycyclopentyl)ethynyl)-1H-indazol-5- | | | | |
| | | yl)pyridin-2-yl)carbamate | | | | |
| 305 | SU1741 | 1-((3-amino-5-(2-(oxetan-3-ylamino)pyridin-4- | 96 | nM | 1090 | nM |
| | | yl)-1H-indazol-7-yl)ethynyl)cyclopentan-1-ol | | | | |
| 306 | SU1742 | methyl (4-(3-amino-7-phenyl-1H-indazol-5- | | | | |
| | | yl)pyridin-2-yl)carbamate | | | | |
| 307 | SU1743 | methyl (4-(3-amino-7-(4- | | | | |
| | | (difluoromethyl)phenyl)-1H-indazol-5- | | | | |
| | | yl)pyridin-2-yl)carbamate | | | | |
| 308 | SU1737 | 7-(3,3-dimethylbut-1-yn-1-yl)-5-(2- | 223 | nM | >3330 | nM |
| | | (trifluoromethyl)-3H-imidazo[4,5-b]pyridin-7- | | | | |
| | | yl)-1H-indazol-3-amine | | | | |
| 309 | SU1770 | N-(4-(3-Amino-7-(3,3-dimethylbut-1-yn-1-yl)-1H- | 421 | nM | 8660 | nM |
| | | indazol-5-yl)pyridin-2-yl)cyanamide | | | | |
| 310 | SU1759 | 5-(2-((1H-Pyrazol-3-yl)amino)pyridin-4-yl)-7- | 48 | nM | 205 | nM |
| | | (3,3-dimethylbut-1-yn-1-yl)-1H-indazol-3- | | | | |
| | | amine | | | | |
| 311 | SU1760 | 5-(2-((1H-Pyrazol-4-yl)amino)pyridin-4-yl)-7- | 10 | nM | 280 | nM |
| | | (3,3-dimethylbut-1-yn-1-yl)-1H-indazol-3- | | | | |
| | | amine | | | | |
| 312 | SU1745 | 7-(3,3-Dimethylbut-1-yn-1-yl)-5-(2-((5-methyl- | 45 | nM | 210 | nM |
| | | 1H-pyrazol-3-yl)amino)pyridin-4-yl)-1H- | | | | |
| | | indazol-3-amine | | | | |
| 313 | SU1750 | 7-(3,3-Dimethylbut-1-yn-1-yl)-5-(2-((3-methyl- | 24 | nM | 940 | nM |
| | | 1H-pyrazol-4-yl)amino)pyridin-4-yl)-1H- | | | | |
| | | indazol-3-amine | | | | |
| 314 | SU1762 | N-(4-(3-Amino-7-(3,3-dimethylbut-1-yn-1-yl)- | 911 | nM | 10,000 | nM |
| | | 1H-indazol-5-yl)pyridin-2-yl)thiazol-2-amine | | | | |
| 315 | SU1746 | N-(4-(3-Amino-7-(3,3-dimethylbut-1-yn-1-yl)- | 54 | nM | 836 | nM |
| | | 1H-indazol-5-yl)pyridin-2-yl)-4-methyloxazol- | | | | |
| | | 2-amine | | | | |
| 316 | SU1753 | N-(4-(3-Amino-7-(3,3-dimethylbut-1-yn-1-yl)- | 335 | nM | >30,000 | nM |
| | | 1H-indazol-5-yl)pyridin-2-yl)-4- | | | | |
| | | (trifluoromethyl)oxazol-2-amine | | | | |
| 317 | SU1764 | N-(4-(3-amino-7-(3,3-dimethylbut-1-yn-1-yl)- | 318 | nM | 4040 | nM |
| | | 1H-indazol-5-yl)pyridin-2-yl)-3,5- | | | | |
| | | dimethylisoxazol-4-amine | | | | |
| 318 | SU1769 | 5-(2-((1H-Imidazol-4-yl)amino)pyridin-4-yl)-7- | 66 | nM | 502 | nM |
| | | (3,3-dimethylbut-1-yn-1-yl)-1H-indazol-3- | | | | |
| | | amine | | | | |
| 319 | SU1751 | 5-(2-((4H-1,2,4-Triazol-3-yl)amino)pyridin-4- | 27 | nM | 486 | nM |
| | | yl)-7-(3,3-dimethylbut-1-yn-1-yl)-1H-indazol-3- | | | | |
| | | amine | | | | |
| 320 | SU1766 | 7-(3,3-Dimethylbut-1-yn-1-yl)-5-(2-((2-methyl- | 5 | nM | 18.5 | nM |
| | | 2H-tetrazol-5-yl)amino)pyridin-4-yl)-1H- | | | | |
| | | indazol-3-amine | | | | |
| 321 | SU1767 | 7-(3,3-Dimethylbut-1-yn-1-yl)-5-(2-(pyrimidin- | 202 | nM | 4580 | nM |
| | | 2-ylamino)pyridin-4-yl)-1H-indazol-3-amine | | | | |
| 322 | SU1763 | 7-(3,3-Dimethylbut-1-yn-1-yl)-5-(2- | 45 | nM | 3130 | nM |
| | | ((tetrahydrofuran-3-yl)amino)pyridin-4-yl)-1H- | | | | |
| | | indazol-3-amine | | | | |
| 323 | SU1744 | Methyl (4-(3-Amino-7-(4-hydroxyphenyl)-1H- | 9.8 | nM | 83 | nM |
| | | indazol-5-yl)pyridin-2-yl)carbamate | | | | |
| 324 | SU1749 | 4-(3-Amino-5-(2-(oxetan-3-ylamino)pyridin-4- | 8090 | nM | 4730 | nM |
| | | yl)-1H-indazol-7-yl)phenol | | | | |
| 325 | SU1755 | Methyl (4-(3-amino-7-(4-aminophenyl)-1H- | 6 | nM | 79 | nM |
| | | indazol-5-yl)pyridin-2-yl)carbamate | | | | |
| 326 | SU1756 | Methyl (4-(7-(4-acetamidophenyl)-3-amino- | 14 | nM | 87 | nM |
| | | 1H-indazol-5-yl)pyridin-2-yl)carbamate | | | | |
| 327 | SU1752 | Methyl (4-(3-amino-7-(4-carbamoylphenyl)- | 614 | nM | 3790 | nM |
| | | 1H-indazol-5-yl)pyridin-2-yl)carbamate | | | | |
| 328 | SU1757 | Methyl (4-(3-amino-7-(4- | 24 | nM | 407 | nM |
| | | (morpholinomethyl)phenyl)-1H-indazol-5- | | | | |
| | | yl)pyridin-2-yl)carbamate | | | | |
| 329 | SU1772 | 5-(2-Aminopyridin-4-yl)-7-(4-(2- | 14 | nM | 108 | nM |
| | | morpholinoethyl)phenyl)-1H-indazol-3-amine | | | | |
| 330 | SU1773 | Methyl (4-(3-amino-7-(4-(2- | >10,000 | nM | >10,000 | nM |
| | | morpholinoethyl)phenyl)-1H-indazol-5- | | | | |
| | | yl)pyridin-2-yl)carbamate | | | | |
| 331 | SU1758 | Methyl (4-(3-amino-7-(4- | | | | |
| | | (methylsulfonyl)phenyl)-1H-indazol-5- | | | | |
| | | yl)pyridin-2-yl)carbamate | | | | |
| 332 | SU1748 | Methyl (4-(3-amino-7-(3-hydroxyphenyl)-1H- | | | | |
| | | indazol-5-yl)pyridin-2-yl)carbamate | | | | |
| 333 | SU1754 | Methyl (4-(3-amino-7-(3-carbamoylphenyl)- | | | | |
| | | 1H-indazol-5-yl)pyridin-2-yl)carbamate | | | | |
| 334 | SU1765 | Methyl (4-(3-amino-7-(3- | 38 | nM | 429 | nM |
| | | (morpholinomethyl)phenyl)-1H-indazol-5- | | | | |
| | | yl)pyridin-2-yl)carbamate | | | | |
| 335 | SU1340 | N-(4-(3-Amino-1H-indazol-5-yl)pyridin-2-yl)-2- | 340 | nM | 4200 | nM |
| | | cyclohexylacetamide | | | | |
| 336 | SU1710 | 6-Amino-4-(3-amino-7-(3,3-dimethylbut-1-yn- | >10,000 | nM | >10,000 | nM |
| | | 1-yl)-1H-indazol-5-yl)nicotinonitrile | | | | |

The IKKα and IKKβ figures quoted in non-italicised form are from the DELFIA assay described above.

The IKKα and IKKb figures quoted in italicised form are from the Adapta assay described above.

Target Engagement Studies in Cells with Exemplars from the Series

### Cell Culture

PC3M cells were grown in RPMI 1640 media which was supplemented with 10% (v/v) foetal calf serum (FCS), L-glutamine (27 mg/ml) and penicillin/streptomycin (250 units/ml; 100 μg/ml). All cells were incubated in a humidified atmosphere at 37° C. and 5% (v/v) CO₂. Cells were cultured as a monolayer in 10 ml media in 75 cm³ vented flasks and grown until subculture was required.

### Sub-Culturing of Cells

Cells were grown as a monolayer until approximately 70-85% confluent, the media was then aspirated, and the cells washed twice with 1.5 ml sterile 5% (w/v) trypsin solution. The trypsin was aspirated, and the flasks were given a gentle tap to ensure cells were fully detached. The flask was then washed with 10 ml media to re-suspend the recovered cells for passage into flasks and plates with fresh media as appropriate.

Treatment of Prostate Cancer PC3M Cell Lines with SU Compounds

Determination of effects of inhibitors on the non-canonical NF-κB pathway: PC3M cells were grown in 12-well plates until approximately 85% confluent and serum starved for 24 h prior to stimulation. After serum starvation, cells were pre-treated with vehicle (V; <0.05% (v/v) DMSO) or increasing concentrations (0.01 to 10 mM) of Example compound (SU inhibitor SU1XXX) for 1 hour prior to exposure to lymphotoxin (LT_{1a2b}) (20 ng/ml) for 4 hours prior to preparation of whole cell extracts.

Determination of effects of inhibitors on the canonical NF-κB pathway: PC3M cells were grown in 12-well plates until approximately 85% confluent and serum starved for 24 h prior to stimulation. After serum starvation, cells were pre-treated with vehicle (V; <0.05% (v/v) DMSO) or increasing concentrations (0.01 to 10 mM) of Example compound (SU inhibitor—SUXXXX) for 1 hour prior to exposure to TNFα (10 ng/ml) for 0.5 h prior to preparation of whole cell extracts.

### Sample Preparation (Whole Cell Extracts (WCEs) and Western Blotting

### Sample Preparation

Whole cell extracts were prepared using a 1× sample buffer as detailed by Laemmli (1970: Nature, 227, 680-5) (63 mM Tris/HCl [pH 6.8], 2 mM Na₄P₂O₇, 5 mM EDTA, 10% (v/v) glycerol, 2% (w/v) SDS, 0.007% (w/v) bromophenol blue and 50 mM DTT; termed DTT-SB). The bathing medium was firstly aspirated, and the cells were washed once with 1 ml of cold PBS. 250 μl of DTT-SB was added to each well to lyse the cells and cellular material was recovered using a cell scraper. Samples were passed through a 21 g needle 3-5 times to shear genomic DNA. Samples were then placed in eppendorfs boiled for 5 min (to denature the protein polypeptides). Samples were either used immediately or frozen at −20° C. for future use.

### SDS-Polyacrylamide Gel Electrophoresis

Prepared protein samples were separated based on their electrophoretic mobility using SDS-polyacrylamide gel electrophoresis (SDS-PAGE). Resolving gel, were prepared using N-methylenebis-acrylamide (30:0.8) to final differing percentages of 10% (v/v) or 7.5% (v/v) acrylamide containing 0.375 M Tris (pH 8.8), 0.1% (w/v) SDS and 10% (w/v) ammonium persulfate (APS) and 10% (v/v) glycerol. The acrylamide gel polymerised at room temperature following the addition of N,N,N,N-tetramethylethylenediamine (TEMED) 0.05% (v/v). The solution was poured between two glass plates with 0.05 cm spacing (Biorad Protean Ill setup) and this was assembled in a vertical slab orientation, leaving a 1-1.5 cm space and overlayed with 0.1% (w/v) SDS until polymerisation had taken place. After the gel had polymerised, the 0.1% (w/v) SDS was removed, and a stacking gel added. The stacking gel was composed of 10% (v/v) acrylamide: N-methylenebis-acrylamide (30:0.8) in 125 mM Tris, (pH 6.8) 0.1% (w/v) SDS, 0.05% (w/v) APS and 0.05% (v/v) TEMED. Immediately following the addition of the stacking gel, a teflon comb (10 or 15 wells) was inserted prior to polymerisation (-10-15 mins) to form the wells for loading. After polymerisation, the combs were removed from the wells, gels assembled in a Protean III™ (Bio-Rad) electrophoresis tank (Bio-Rad) and filled with electrophoresis running buffer (25 mM Tris, pH 7.5, 129 mM glycine, 0.1% (w/v) SDS). Using a Hamilton™ micro-syringe, a volume (2-5 μl) of pre-stained molecular weight (MW) markers of known molecular weights was loaded into a well in parallel to prepared cell lysates. Samples were electrophoresed at a constant voltage of 130 V until the bromophenol dye present in the sample buffer migrated beyond the bottom of the gel.

### Electrophoretic Transfer of Proteins to a Nitrocellulose Membrane

Following gel electrophoresis, gels containing separated protein polypeptides were transferred onto nitrocellulose membranes by electrophoretic blotting. Each gel was placed firmly onto a nitrocellulose sheet between two 3 mm sheets of blotting paper and two outer sponges and assembled in a transfer cassette. This was submerged in transblot buffer (25 M Tris, 19 mM glycine, 20% (v/v) methanol) in a Bio-Rad Mini Trans-Blot™ tank with the nitrocellulose facing towards the anode. A constant current of 300 mA was applied for 1.75 h whilst cooled by the addition of an ice pack to the transfer tank.

### Immunological Detection of Protein Using Antisera

Following transfer of the proteins to the nitrocellulose membrane, the membrane was then ‘blocked’ for non-specific binding by incubation in 5% (w/v) bovine serum albumin (BSA) in Tris-buffered saline, 0.1% Tween (TBST) buffer blocking solution (150 mM NaCl, 20 mM Tris, pH7.4, 0.1% (v/v) Tween-20) and gently rocked back and forth on a platform shaker for 2 h. The 5% (w/v) BSA blocking buffer was then discarded and replaced with 0.5% (w/v) BSA TBST pH 7.4 solution containing an appropriate concentration of antisera (primary antibody) specific to the target protein. This was left overnight on a roller at 4° C. in the cold room. The next day, the membranes were washed every 5 minutes in TBST for 15 minutes on a platform shaker. After this wash cycle, the membranes were incubated with a secondary antibody, an IgG antibody (conjugated with horseradish peroxidase) raised against the species of the primary antibody. This was added (1:10,000) to 0.5% (w/v) BSA in TBST buffer (pH 7.4) and left on the platform shaker at room temperature for 1.5 h. Following this, the membrane was then washed every 5 min for 15 min with TBST as described before. After this second wash cycle, the membrane was then developed using enhanced chemi-luminescence (ECL) reagents. The TBST buffer from the final wash was discarded and 5 ml of both ECL solution 1 (2.5 mM Luminol, 1.2 mM coumaric acid and 100 mM Tris/HCl solution [pH 8.5]) and 5 ml ECL solution 2 (100 mM Tris/HCl solution [pH 8.5] and 6.27 mM H₂O₂) were added and washed over the membrane for 2 min. The membranes were then blotted on tissue (to remove an excess ECL solution) before placed in an exposure cassette and covered with cling film. Lastly, the membranes were developed in the dark room where Kodak X-OMAT LS film was exposed to the membranes for an appropriate amount of time, dependent on the sensitivity of the antibody used. The film was developed by a Kodak M35-M-X-OMAT processor. The films were then scanned and quantified by densitometry using Scion image software (Scion Corp, Maryland, USA).

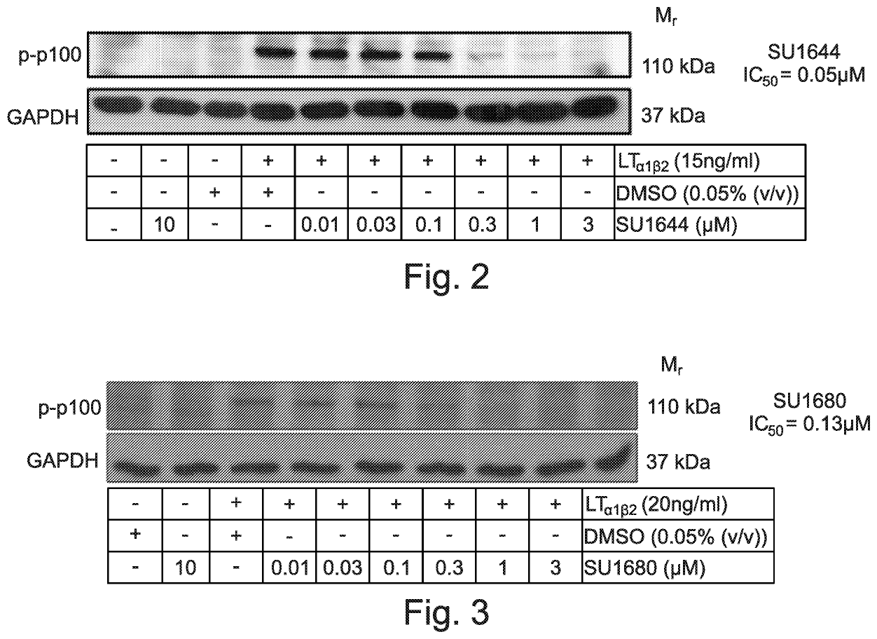

### Results—FIGS. 2-9

Whole cell lysates were prepared for separation using SDS-PAGE and analysed by Western blotting (see above) using specific antibodies for proteins relevant to the non-canonical and canonical NF-κB signalling pathways. GAPDH was used as a loading control. Blots from at least three independent experiments were quantified by scanning densitometry, each normalised to GAPDH expression and calculated as mean±S.E.M relative to the stimulated sample (LT_{1a2b} plus vehicle (4h; for non-canonical NF-kB markers) or TNFα plus vehicle (30 min; for canonical NF-kB markers)). Following curve fitting, IC₅₀ values were determined.

FIG. 2—Effect of compound Example 164 (SU1644) on lymphotoxin-stimulated p100 phosphorylation in PC3M cells. Cells were pre-treated with SU1644 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

FIG. 3. Effect of compound Example 190 (SU1680) on lymphotoxin-stimulated p100 phosphorylation in PC3M cells. Cells were pre-treated with Example 190 (SU1680) 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

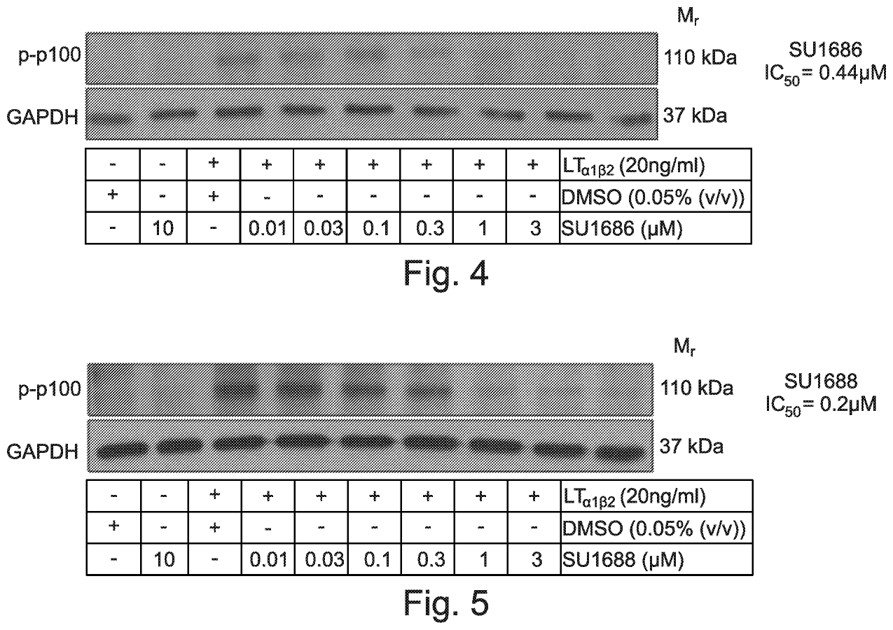

FIG. 4. Effect of compound Example 186 (SU1686) on lymphotoxin-stimulated p100 phosphorylation in PC3M cells. Cells were pre-treated with Example 186 (SU1686) 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

FIG. 5. Effect of compound Example 167 (SU1688) on lymphotoxin-stimulated p100 phosphorylation in PC3M cells. Cells were pre-treated with Example 167 (SU1688) 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

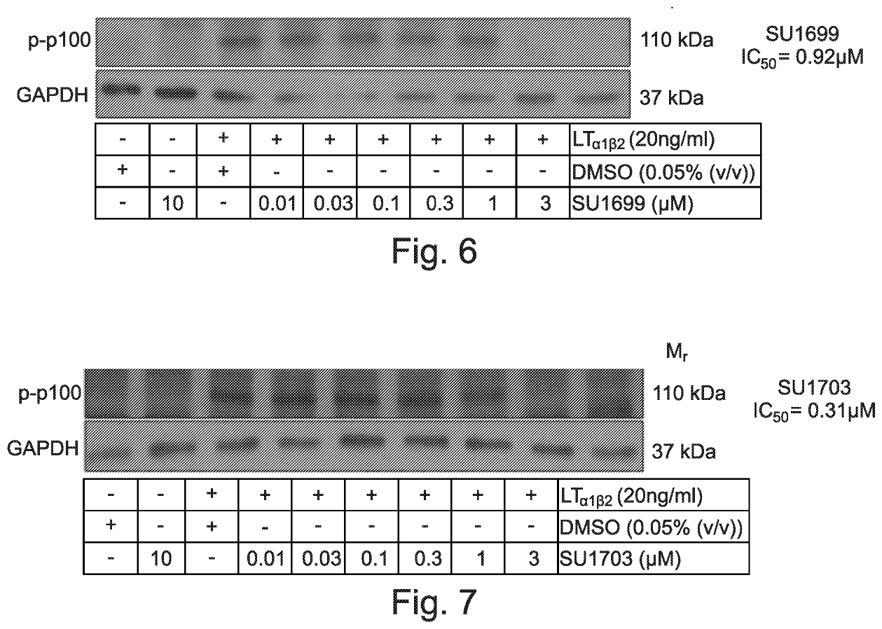

FIG. 6. Effect of compound Example 155 (SU1699) on lymphotoxin-stimulated p100 phosphorylation in PC3M cells. Cells were pre-treated with Example 155 (SU1699) 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

FIG. 7. Effect of compound Example 180 (SU1703) on lymphotoxin-stimulated p100 phosphorylation in PC3M cells. Cells were pre-treated with Example 180 (SU1703) 1 h prior to stimulation with lymphotoxin for 4 h. Whole cell lysates were prepared and separated by SDS-PAGE. Blots were quantified from three independent experiments, the level of protein normalised against GAPDH and the IC₅₀ was determined.

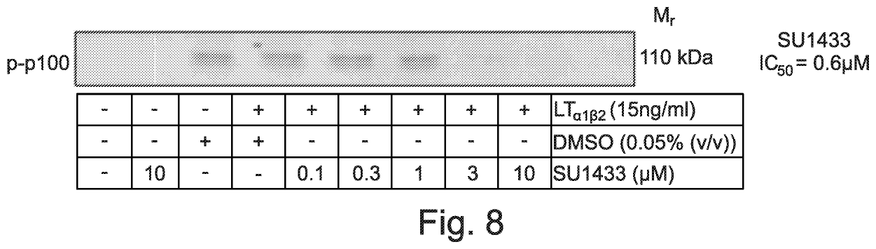

FIG. 8. Effect of compound Example 137 (SU1433) on lymphotoxin-stimulated p100 phosphorylation in PC3M cells. Cells were pre-treated with Example 137 (SU1433) 1 h prior to stimulation with lymphotoxin for 4 h. Whole cell lysates were prepared and separated by SDS-PAGE. Blots were quantified from three independent experiments, the level of protein normalised against GAPDH (not shown) and the IC₅₀ was determined.

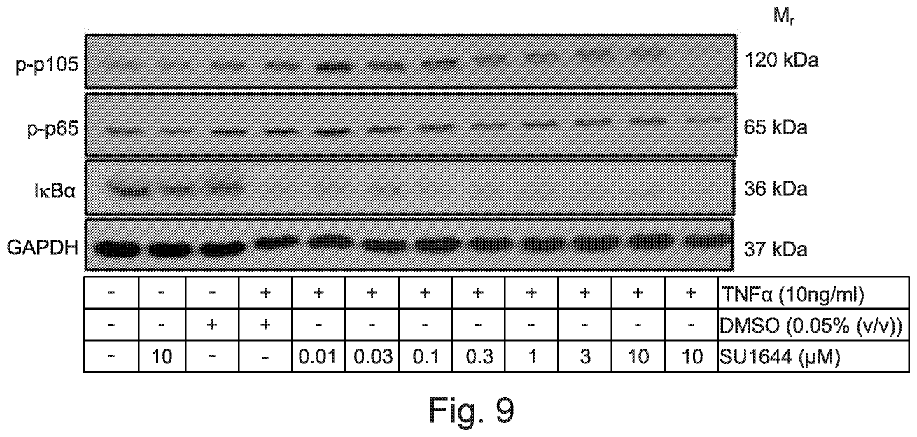

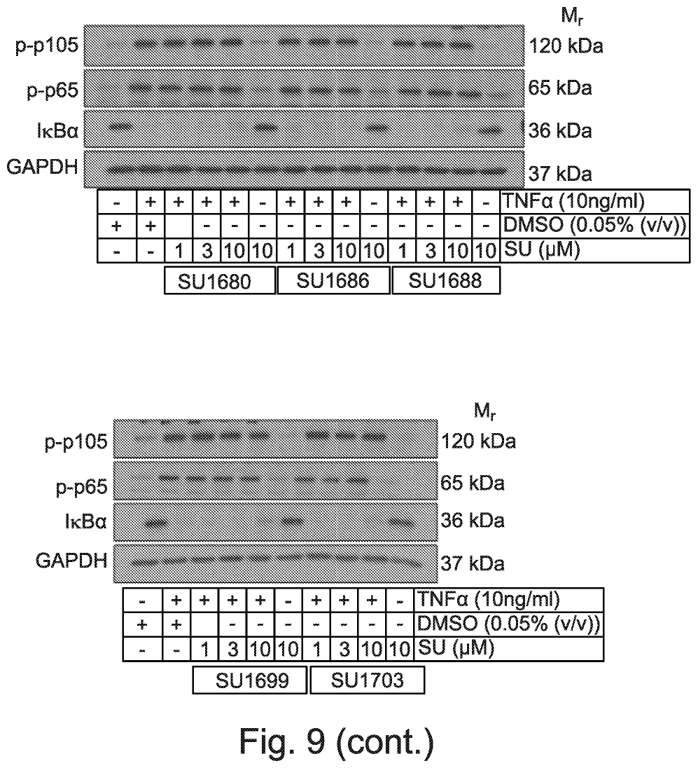

FIG. 9. Effect of compounds Example 164 (SU1644), Example 190 (SU1680), Example 186 (SU1686), Example 167 (SU1688), Example 155 (SU1699) and Example 180 (SU1703) on the TNFa-stimulated phosphorylation of p105 and p65 and IkBa degradation in PC3M cells. Cells were pre-treated with the compounds 1 h prior to stimulation with TNFα for 0.5 h. Whole cell lysates were prepared and separated by SDS-PAGE with GAPDH used as a loading control. The results are representative of two independent experiments.

Phenotypic Effects in Cells with Exemplars from the Series

### Cell Culture

PC3, PC3M, LNCaP Al, cells were grown in RPMI 1640 media and PANC1 and MiaPac-2 cells were grown in Dulbecco's Modified Eagle's Media (DMEM) which was supplemented with 10% (v/v) Foetal Calf Serum (FCS) (Charcoal-stripped for LNCaP Als), L-glutamine (27 mg/ml) and penicillin/streptomycin (250 units/ml; 100 μg/ml). All cells were incubated in a humidified atmosphere at 37° C. and 5% (v/v) CO₂. Cells were cultured as a monolayer in 10 ml media in 75 cm³ vented flasks and grown until subculture was required.

### Sub-Culturing of Cells

Cells were grown as a monolayer until approximately 70-85% confluent, the media was then aspirated, and the cells washed twice with 1.5 ml sterile 5% (w/v) trypsin solution. The trypsin was aspirated, and the flasks were given a gentle tap to ensure cells were fully detached. The flask was then washed with 10 ml media to re-suspend the recovered cells for passage into flasks and plates with fresh media as appropriate.

Cell viability determination: Cells (1000 cells/100 μl) were plated into 96 well plates and incubated for 24 hours at 37° C. and 5% (v/v) CO₂ before being treated with SU1433. After incubation with SU1433 for 48 h, cell viability was measured using the alamarBlue® Assay (Thermofisher, UK). The media was removed and replaced with a 1:10 media to alamar blue solution. After a 1 h incubation at 37° C. and 5% CO₂, the fluorescence was measured at 530-560 nm excitation wavelength and 590 nm emission wavelength in a fluorescence plate reader. All treatment were measured in duplicate wells and repeated at least 3 times. The results represent the fractional survival of untreated controls±SD.

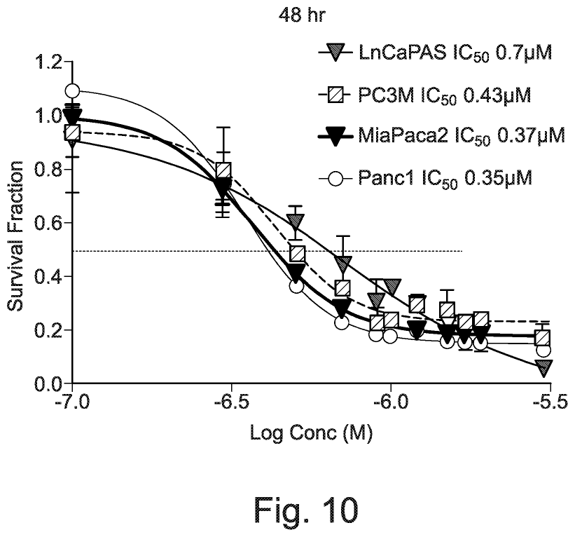

### Results—FIG. 10

FIG. 10. Treatment with Example 137 (SU1433) for 48 hours reduced the viability of PC3M, LNCaP, PANC1 and MiaPaca-2 cells.

Cellular clonogenic capacity determination: Cells were plated into 6 well plates at a density of 125 cells/well and incubated for 24 hat 37° C. and 5% (v/v) CO₂ before being treated with Example compound (SU1XXX). For treatment all media was removed and replaced with Example compound (SUXXXX) (diluted in media). To allow colony formation the cells were incubated for 8 days at 37° C. and 5% (v/v) CO₂ before being fixed in 100% methanol and visualised by staining with a solution of 1% (v/v) Giemsa (BDH Laboratory Supplies) and counted. All treatments were measured in duplicate wells and repeated at least 3 times. The results represent the fractional survival of untreated controls±SD.

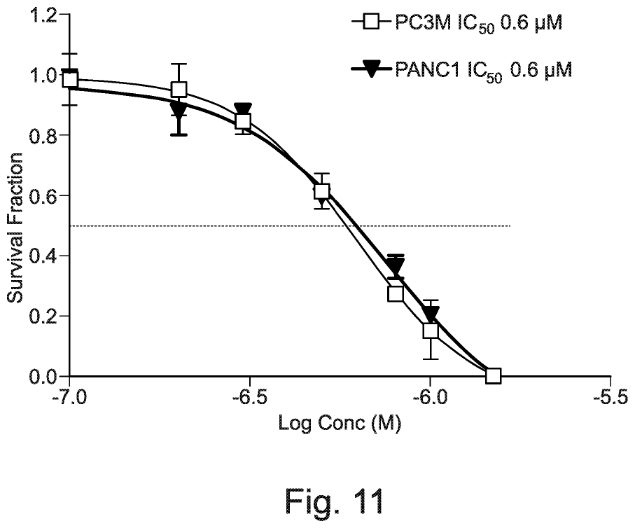

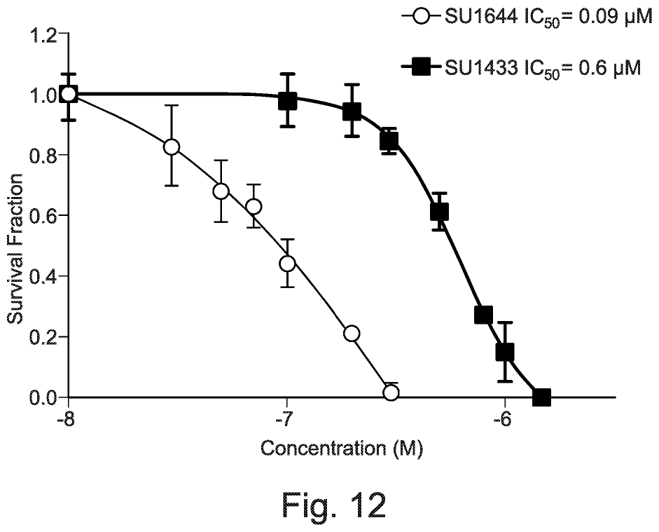

### Results—FIGS. 11 and 12

FIG. 11. The effect of Example 137 (SU1433) on the clonogenic capacity of PC3M and PANC1 cells following 8-day continuous treatment.

FIG. 12. The effect of Example 137 (SU1433) and Example 164 (SU1644) on the clonogenic capacity of PC3M ells following 8-day continuous treatment.

### In Vivo Assessment of Example 137 (SU1433) in Nude Mice Bearing PC3M Xenografts

Xenografts were established in nude mice by subcutaneous injection of 5×10⁶ PC3M-Luc-C6 cells. After 8 days, mice bearing tumours of approximately 60 mm³ in volume were randomised into 3 treatment groups of 8 mice in each. One group of mice received intraperitoneal injection (i.p.) of 50 mg/kg SU1433 once daily dissolved in a vehicle of 5% (v/v) DMSO, 5% (v/v) solutol HS15 and 15% (w/v) hydroxypropyl-p-cyclodextrin in water for injection (90%) for a total of 21 days. Another group received once daily i.p. injection of the vehicle (100 μL) alone whilst the last group received no treatment. To monitor potential toxicity, body weight was measured daily, and experimental animals were evaluated for signs of distress using standard guidelines. Mice whose xenografts reached 1,900 mm³ were euthanised. Subcutaneous tumours were measured with calipers immediately before treatment and every 2 or 3 days thereafter. On the assumption of ellipsoidal geometry, diameter measurements were converted to an approximate volume by multiplying half the longest diameter by the square of the mean of the 2 shorter diameters. For every animal, relative tumour volume (volume at any time point divided by volume immediately before treatment) was plotted against time, and the area under the time-volume curves was determined by trapezoidal approximation. The area under the time-volume curves was used as a measure of treatment effectiveness for the purpose of comparison between groups. To test for differences in tumour growth between experimental therapy groups, the Mann-Whitney U test with Dunn's multiple comparisons test was used.

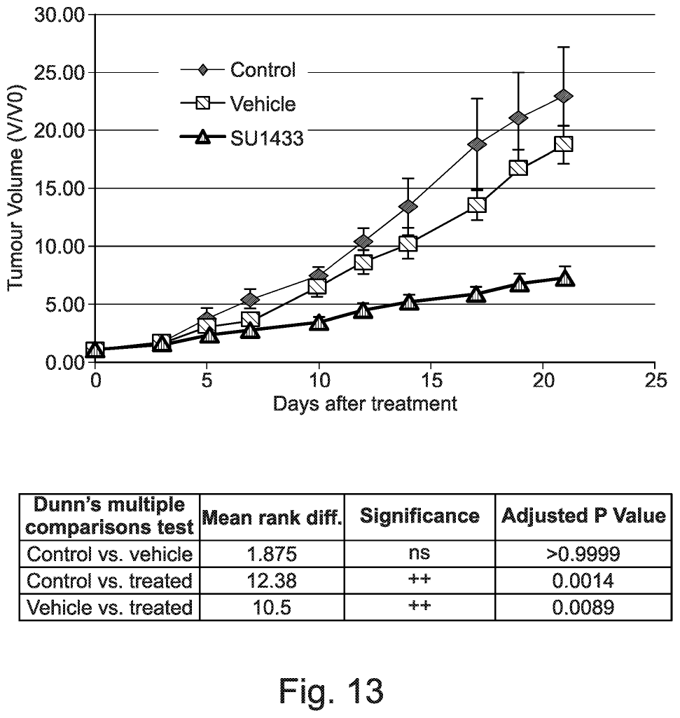

### Results—FIG. 13

FIG. 13. Example 137 (SU1433) inhibits tumour growth in nude mice bearing PC3M xenografts by approximately 60% over 21 days with a once daily i.p. dose of 50 mg/kg

1. A compound, or a pharmaceutically acceptable salt, hydrate or solvate thereof, having the structural Formula (I), shown below: 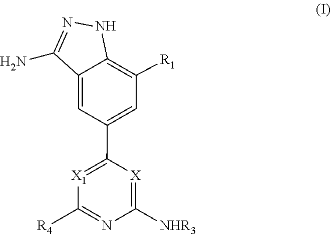
wherein:
R₁ is selected from hydrogen, halogen, (1-6C)alkyl, (2-6C)alkynyl, (3-7C) cycloalkyl, aryl, heteroaryl and heterocyclyl,
and wherein said (1-6C)alkyl, (2-6C)alkynyl, (3-7C) cycloalkyl, aryl, heteroaryl and heterocyclyl are optionally substituted by one or more R₁₀₀ substituents;
wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₙ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}heterocyclyl, (CH₂)_{z}heteroaryl, or (CH₂)_{z}aryl;
and wherein:
(i) R_{f} and R_{g} are each independently selected from hydrogen, (1-6C)alkyl or phenyl; and wherein Rₕ and Rⱼ are each independently selected from hydrogen, (1-6C)alkyl or phenyl or Rₕ and Rⱼ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (1-4C)alkyl, (3-7C)cycloalkyl, heterocyclyl, heteroaryl or aryl in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl;
X is N or CR₂; wherein R₂ is selected from hydrogen, halogen, (1-8C)alkyl, (2-8C)alkenyl, (2-8C)alkynyl, (3-7C)cycloalkyl, aryl, heteroaryl and heterocyclyl,
wherein said (1-8C)alkyl, (2-8C)alkenyl, (2-8C)alkynyl, (3-7C)cycloalkyl, aryl, heteroaryl and heterocyclyl are optionally substituted by one or more R₂₀₀ substituents;
wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}aryl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or aryl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rr, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl;
R₃ is selected from hydrogen, cyano, (1-8C)alkyl, (3-7C)cycloalkyl, (CH₂)₁₋₃(3-7C)cycloalkyl, a carbon-linked 4 to 7 membered heterocyclyl, a carbon-linked 5 to 6 membered heteroaryl, —C(O)-(1-8C)alkyl, —C(O)(CH₂)₀₋₃(3-7C)cycloalkyl, —C(O)[5 or 6-membered heteroaryl], —C(O)phenyl, —C(O)O(1-8C)alkyl, —C(O)O(3-7C)cycloalkyl, —C(O)O(CH₂)₁₋₃(3-7C)cycloalkyl, —C(O)NH₂, —C(O)NH-(1-8C)alkyl, —C(O)NH—(CH₂)₀₋₃(3-7C)cycloalkyl, —C(O)NH—(CH₂)₀₋₃heterocyclyl, —C(O)NH—(CH₂)₀₋₃[5 or 6-membered heteroaryl], —C(O)NH—(CH₂)₀₋₃phenyl, —S(O)₂H or —S(O)₂-(1-8C)alkyl;
wherein any alkyl, cycloalkyl, phenyl, heteroaryl or heterocyclyl moiety is optionally substituted by one or more R₃₀₀ substituents;
wherein R₃₀₀ is selected from halo, trifluoromethyl, trifluoromethoxy, cyano, hydroxyl, (1-4C)alkyl, (1-4C)hydroxyalkyl, (CH₂)_{z}ORₛ, (CH₂)_{z}C(O)Rₛ, (CH₂)_{z}C(O)ORₛ, (CH₂)_{z}OC(O)Rₛ, (CH₂)_{z}C(O)N(R_{y})Rᵤ, (CH₂)_{z}N(Rₜ)C(O)Rₛ, (CH₂)_{z}N(Rₜ)C(O)ORₛ, (CH₂)_{z}S(O)_{y}Rₛ, (CH₂)_{z}SO₂N(Rᵥ)Rᵤ, (CH₂)_{z}N(Rₜ)SO₂Rₛ, (CH₂)_{z}NRᵤRᵥ;
and wherein:
(i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms, and wherein any 3-7 membered ring formed Rₒ and Rₚ, and any alkyl or phenyl group present for Rₛ, Rₜ, Rᵤ and Rᵥ is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any alkyl, cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{w}, C(O)R_{w}, C(O)OR_{w}, OC(O)R_{w}, C(O)N(R_{w})Rₓ, N(Rₓ)C(O)R_{w}, S(O)_{y}R_{w}, SO₂N(Rₓ)R_{w}, N(Rₓ)SO₂R_{w}, or NR_{w}Rₓ, wherein R_{w} is hydrogen, (1-2C)alkyl or phenyl, and Rₓ are selected from hydrogen or (1-2C)alkyl;
or R₂ and R₃ are linked such that together they form a —CH═CQ- or —N═CQ- group;
Q is hydrogen, halo, cyano or a group of the formula:
-L₁-Y₁-L₂-Q₁
wherein:
L₁ is absent or (1-3C)alkylene;
Y₁ is absent or O, S, SO, SO₂, N(R_{y1}), C(O), C(O)O, OC(O), C(O)N(R_{y1}), or N(R_{y1})C(O), wherein R_{y1} is selected from hydrogen or (1-4C)alkyl;
L₂ is absent or (1-3C)alkylene; and
Q₁ is hydrogen, (1-6C)alkyl, (2-6C)alkenyl, (2-6C)alkynyl, phenyl, (3-8C)cycloalkyl, heteroaryl or heterocyclyl;
wherein Q is optionally further substituted by one or more substituent groups independently selected from oxo, hydroxy, (1-6C)alkyl, halo, (1-4C)haloalkyl, (1-4C)haloalkoxy, (1-4C)aminoalkyl, (1-4C)hydroxyalkyl, cyano, or by one or more group(s) of the formula:
-L₃-Y₂-L₄-W₁
wherein:
L₃ is absent or (1-3C)alkylene;
Y₂ is absent or selected from or O, S, SO, SO₂, N(R_{y2}), C(O), C(O)O, OC(O), C(O)N(R_{y2}), or N(R_{y2})C(O), S(O)₂N(R_{y2}), N(R_{y2})SO₂ wherein R_{y2} is selected from hydrogen or (1-3C)alkyl;
L₄ is absent or (1-3C)alkylene; and
W₁ is hydrogen, (1-6C)alkyl, phenyl, (3-8C)cycloalkyl, heteroaryl or heterocyclyl;
wherein W₁ is optionally substituted by one or more substituents selected from oxo, (1-4C)alkyl, halo, (1-4C)haloalkyl, (1-4C)haloalkoxy, (1-4C)alkoxy, amino, (1-4C)alkylamino, di[(1-4C)alkyl]amino C(O)OH, C(O)O(1-4C)alkyl, (CH₂)₀₋₃-heterocyclyl or cyano;
R₄ is selected from hydrogen, halo, cyano or amino;
X₁ is N when R₂ and R₃ are linked such that together they form a —CH═CH— group; or CR₅ wherein R₅ is selected from hydrogen, halo, cyano or amino;
y is independently selected from 0, 1 or 2;
z is independently selected from 0, 1, 2 or 3;
with the proviso that:
(i) R₁ is not hydrogen when R₂ and R₃ are both hydrogen;
(ii) R₂ is not hydrogen when R₁ and R₃ are both hydrogen;
(iii) R₁ is not hydrogen when R₂ and R₃ are linked to form a —CH═CH— group;
(iv) R₁ is not hydrogen when X is N and R₃ is hydrogen; and
(v) Q is not aryl or heteroaryl when all of L₁, Y₁ and L₂ are absent.

2. A compound according to claim 1, or a pharmaceutically acceptable salt or solvate thereof, wherein the compound is a compound having the structural formula (Ia), (Ib), (Ic), (Id) or (Ie) shown below: 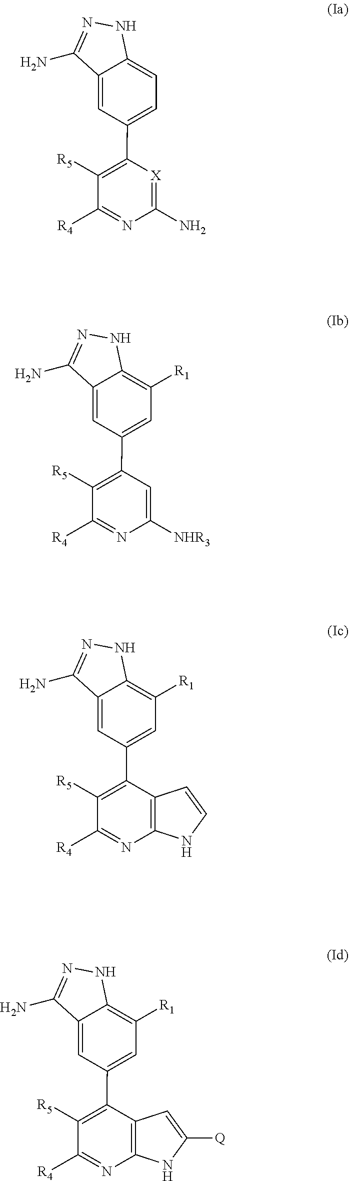 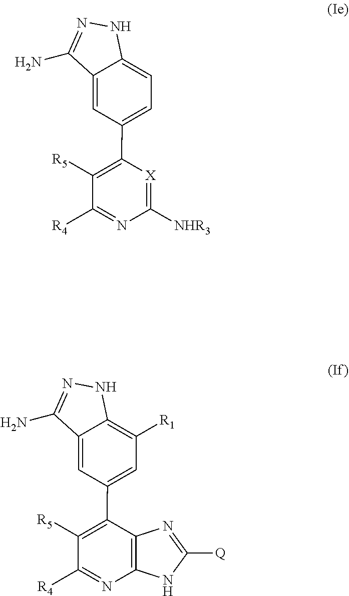
wherein R₁, X, R₃, R₄, R₅ and Q are each as defined in claim 1.

3. A compound according to claim 1 or claim 2, or a pharmaceutically acceptable salt or solvate thereof, wherein R₁ is selected from hydrogen, halogen, (1-6C)alkyl, (2-6C)alkynyl, (3-7C)cycloalkyl, phenyl, a 5 or 6-membered heteroaryl or a 4 to 7-membered heterocyclyl,
wherein said (2-6C)alkynyl, (3-7C)cycloalkyl, phenyl, heteroaryl and heterocyclyl are optionally substituted by one or more R₁₀₀ substituents;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}heterocyclyl, (CH₂)_{z}heteroaryl, or (CH₂)_{z}phenyl;
and wherein:
(i) R_{f} and R_{g} are each independently selected from hydrogen or (1-2C)alkyl; and wherein Rₕ and Rⱼ are each independently selected from hydrogen or (1-2C)alkyl or Rₕ and Rⱼ together with the nitrogen atom to which they are attached form a 3-7 membered ring which may optionally include further heteroatoms; and
(ii) any (1-4C)alkyl, (3-7C)cycloalkyl, heterocyclyl, heteroaryl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl.

4. A compound according to any one of the preceding claims, or a pharmaceutically acceptable salt or solvate thereof, wherein R₁ is selected from hydrogen, halogen, (1-6C)alkyl, (2-6C)alkynyl, phenyl or a 5 or 6-membered heteroaryl,
wherein said (2-6C)alkynyl, phenyl or heteroaryl are optionally substituted by one or more R₁₀₀ substituents;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}-[4-6 membered heterocyclyl], (CH₂)_{z}-[5 or 6 membered heteroaryl] or (CH₂)_{z}phenyl;
and wherein:
(i) R_{f} and R_{g} are each independently selected from hydrogen or (1-2C)alkyl; and wherein Rₕ and Rⱼ are each independently selected from hydrogen or (1-2C)alkyl or Rₕ and Rⱼ together with the nitrogen atom to which they are attached form a 3-7 membered ring which may optionally include further heteroatoms; and
(ii) any (1-4C)alkyl, (3-7C)cycloalkyl, heterocyclyl, heteroaryl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl ORₖ, C(O)Rₖ, C(O)ORₖ, OC(O)Rₖ, C(O)N(Rₗ)Rₖ, N(Rₗ)C(O)Rₖ, S(O)_{y}Rₖ, SO₂N(Rₗ)Rₖ, N(Rₗ)SO₂Rₖ, or NRₗRₖ, wherein Rₖ and Rₗ are selected from hydrogen or (1-2C)alkyl.

5. A compound according to any one of the preceding claims, or a pharmaceutically acceptable salt or solvate thereof, wherein
R₁ is selected from hydrogen, halogen, (1-6C)alkyl, (2-6C)alkynyl, phenyl or a 5 or 6-membered heteroaryl,
wherein said (2-6C)alkynyl, phenyl or heteroaryl are optionally substituted by one or more R₁₀₀ substituents;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, (CH₂)_{z}C(O)R_{f}, (CH₂)_{z}C(O)OR_{f}, (CH₂)_{z}OC(O)R_{f}, (CH₂)_{z}C(O)N(Rⱼ)Rₕ, (CH₂)_{z}N(R_{g})C(O)R_{f}, (CH₂)_{z}S(O)_{y}R_{f}, (CH₂)_{z}SO₂N(Rⱼ)Rₕ, (CH₂)_{z}N(R_{g})SO₂R_{f}, (CH₂)_{z}NRⱼRₕ, (CH₂)_{z}(3-7C)cycloalkyl, (CH₂)_{z}-[4-6 membered heterocyclyl], (CH₂)_{z}-[5 or 6 membered heteroaryl] or (CH₂)_{z}phenyl;
and wherein:
(i) R_{f} and R_{g} are each independently selected from hydrogen or (1-2C)alkyl; and
(ii) any (1-4C)alkyl, (3-7C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl.

6. A compound according to any one of the preceding claims, or a pharmaceutically acceptable salt or solvate thereof, wherein R₁ is selected from hydrogen, (2-6C)alkynyl, phenyl or a 5 or 6-membered heteroaryl,
wherein said (2-6C)alkynyl, phenyl or heteroaryl are optionally substituted by one or more R₁₀₀ substituents;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
and wherein:
(i) R_{f} and R_{g} are each independently selected from hydrogen or (1-2C)alkyl; and
(ii) any (1-4C)alkyl, heterocyclyl, or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl.

7. A compound according to any one of the preceding claims, or a pharmaceutically acceptable salt or solvate thereof, wherein R₁ is selected from:
(i) hydrogen;
(ii) halo;
(iii) methyl;
(iv) CF₃;
(v) ethynyl, i.e. 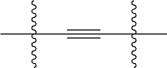 [SMILES: *C#C*]
which is optionally substituted by R₁₀₀;
(vi) phenyl, which is optionally substituted by R₁₀₀;
(vii) a 5 or 6-membered heteroaryl, which is optionally substituted by R₁₀₀;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
and wherein:
(i) R_{f} and R_{g} are each independently selected from hydrogen or (1-2C)alkyl; and
(ii) any (1-4C)alkyl, (3-7C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl.

8. A compound according to any one of the preceding claims, or a pharmaceutically acceptable salt or solvate thereof, wherein R₁ is selected from:
(i) hydrogen;
(ii) halo;
(iii) methyl;
(iv) CF₃;
(v) ethynyl, i.e. 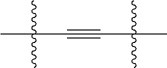 [SMILES: *C#C*]
which is optionally substituted by R₁₀₀;
(vi) phenyl, which is optionally substituted by R₁₀₀;
(vii) a 5 or 6-membered heteroaryl, which is optionally substituted by R₁₀₀;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
and wherein:
(i) R_{f} and R_{g} are each independently selected from hydrogen or (1-2C)alkyl; and
(ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl.

9. A compound according to any one of the preceding claims, or a pharmaceutically acceptable salt or solvate thereof, wherein R₁ is selected from:
(i) hydrogen;
(ii) ethynyl, i.e. 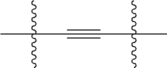 [SMILES: *C#C*]
which is optionally substituted by R₁₀₀;
(iii) phenyl, which is optionally substituted by R₁₀₀;
(iv) a 5 or 6-membered heteroaryl;
and wherein R₁₀₀ is selected from halo, trifluoromethyl, trifluoromethoxy, cyano, hydroxyl, (1-4C)alkyl, (1-4C)hydroxyalkyl, (CH₂)_{z}OR_{f}, C(O)R_{f}, C(O)OR_{f}, OC(O)R_{f}, C(O)N(Rⱼ)Rₕ, N(R_{g})C(O)R_{f}, S(O)_{y}R_{f}, SO₂N(Rⱼ)Rₕ, N(R_{g})SO₂R_{f}, NRⱼRₕ, (CH₂)_{z}-[4-6 membered heterocyclyl], or (CH₂)_{z}phenyl;
and wherein:
(i) R_{f} and R_{g} are each independently selected from hydrogen or (1-2C)alkyl; and
(ii) any (1-4C)alkyl, heterocyclyl or phenyl moiety in a R₁₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl or ORₖ, wherein Rₖ is selected from hydrogen or (1-2C)alkyl;

10. A compound according to any one of the preceding claims, or a pharmaceutically acceptable salt or solvate thereof, wherein X is N or CR₂; wherein R₂ is selected from hydrogen, halogen, (1-8C)alkyl, (2-8C)alkenyl, (2-8C)alkynyl, (3-7C)cycloalkyl, phenyl, a 5 or 6-membered heteroaryl or a 4 to 7-membered heterocyclyl,
wherein said (1-6C)alkyl, (2-6C)alkenyl, (2-6C)alkynyl, (3-7C)cycloalkyl, phenyl, a 5 or 6-membered heteroaryl or a 4 to 7-membered heterocyclyl are optionally substituted by one or more R₂₀₀ substituents;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

11. A compound according to any one of the preceding claims, or a pharmaceutically acceptable salt or solvate thereof, wherein X is N or CR₂; wherein R₂ is selected from hydrogen, halogen, (1-8C)alkyl, (2-8C)alkynyl, (3-7C)cycloalkyl, phenyl, a 5 or 6-membered heteroaryl or a 4 to 7-membered heterocyclyl,
wherein said (2-6C)alkynyl, (3-7C)cycloalkyl, phenyl, heteroaryl and heterocyclyl are optionally substituted by one or more R₂₀₀ substituents;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

12. A compound according to any one of the preceding claims, or a pharmaceutically acceptable salt or solvate thereof, wherein
X is N or CR₂; wherein R₂ is selected from hydrogen, fluoro, (1-8C)alkyl, (2-8C)alkynyl, (3-7C)cycloalkyl, phenyl, or a 5 or 6-membered heteroaryl,
wherein said (1-6C)alkynyl, (3-7C)cycloalkyl, phenyl, heteroaryl and heterocyclyl are optionally substituted by one or more R₂₀₀ substituents;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

13. A compound according to any one of the preceding claims, or a pharmaceutically acceptable salt or solvate thereof, wherein X is N or CR₂; wherein R₂ is selected from hydrogen, fluoro, (1-8C)alkyl, (3-7C)cycloalkyl, or (2-6C)alkynyl,
wherein said (2-6C)alkynyl is optionally substituted by one or more R₂₀₀ substituents;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚare each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

14. A compound according to any one of the preceding claims, or a pharmaceutically acceptable salt or solvate thereof, wherein X is N or CR₂; wherein R₂ is selected from:
(i) hydrogen;
(ii) fluoro;
(iii) ethynyl, i.e. 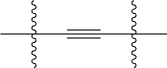 [SMILES: *C#C*]
which is optionally substituted by R₂₀₀;
(vi) phenyl, which is optionally substituted by R₂₀₀;
(vii) a 5 or 6-membered heteroaryl, which is optionally substituted by R₂₀₀;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

15. A compound according to any one of the preceding claims, or a pharmaceutically acceptable salt or solvate thereof, wherein X is N or CR₂; wherein R₂ is selected from:
(i) hydrogen; or
(ii) ethynyl, i.e. 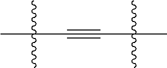 [SMILES: *C#C*]
which is optionally substituted by R₂₀₀;
and wherein R₂₀₀ is selected from halo, trifluoromethyl, trifluoromethoxy, cyano, hydroxyl, (1-4C)alkyl, (1-4C)hydroxyalkyl, (CH₂)_{z}ORₘ, (CH₂)_{z}C(O)Rₘ, (CH₂)_{z}C(O)ORₘ, (CH₂)_{z}OC(O)Rₘ, (CH₂)_{z}C(O)N(Rₒ)Rₚ, (CH₂)_{z}N(Rₙ)C(O)Rₘ, (CH₂)_{z}S(O)_{y}Rₘ, (CH₂)_{z}SO₂N(Rₒ)Rₚ, (CH₂)_{z}N(Rₙ)SO₂Rₘ, (CH₂)_{z}NRₒRₚ, (CH₂)_{z}(3-7C)cycloalkyl, (CH₂)_{z}heterocyclyl, (CH₂)_{z}heteroaryl, or (CH₂)_{z}phenyl;
and wherein:
(i) Rₘ and Rₙ are each independently selected from hydrogen, (1-6C)alkyl or phenyl; Rₒ and Rₚ are each independently selected from hydrogen, (1-6C)alkyl or phenyl or Rₒ and Rₚ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any (3-7C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2C)alkyl, (1-2C)haloalkyl, (1-2C)hydroxyalkyl, OR_{q}, C(O)R_{q}, C(O)OR_{q}, OC(O)R_{q}, C(O)N(R_{q})Rᵣ, N(Rᵣ)C(O)R_{q}, S(O)_{y}R_{q}, SO₂N(Rᵣ)R_{q}, N(Rᵣ)SO₂R_{q}, or NRᵣR_{q}, wherein R_{q} is hydrogen, (1-2C)alkyl or phenyl, and Rᵣ are selected from hydrogen or (1-2C)alkyl.

16. A compound according to any one of the preceding claims, or a pharmaceutically acceptable salt or solvate thereof, wherein X is CR₂.

17. A compound according to any one of the preceding claims, or a pharmaceutically acceptable salt or solvate thereof, wherein R₃ is selected from hydrogen, cyano, (1-8C)alkyl, (3-7C)cycloalkyl, a carbon-linked 4 to 7 membered heterocyclyl, a carbon-linked 5 to 6 membered heteroaryl, —(CH₂)₁₋₃(3-7C)cycloalkyl, —C(O)—(1-8C)alkyl, —C(O)—(CH₂)₀₋₃(3-7C)cycloalkyl, —C(O)phenyl, —C(O)O(1-8C)alkyl, —C(O)NH₂, —C(O)NH—(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl;
wherein any alkyl, cycloalkyl, phenyl, or heteroaryl moiety is optionally substituted by one or more R₃₀₀ substituents;
wherein R₃₀₀ is selected from halo, trifluoromethyl, trifluoromethoxy, cyano, hydroxyl, (1-4C)alkyl, (1-4C)hydroxyalkyl, ORₛ, C(O)Rₛ, C(O)ORₛ, OC(O)Rₛ, C(O)N(Rᵥ)Rᵤ, N(Rₜ)C(O)Rₛ, N(Rₜ)C(O)ORₛ, S(O)_{y}Rₛ, SO₂N(Rᵥ)Rᵤ, N(Rₜ)SO₂Rₛ, (CH₂)_{z}NRᵤRᵥ;
and wherein:
(i) Rₛ and Rₜ are each independently selected from hydrogen, (1-6C)alkyl or (CH₂)_{z}phenyl; Rᵤ and R_{y} are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms, and wherein any 3-7 membered ring formed Rₒ and Rₚ, and any alkyl or phenyl group present for Rₛ, Rₜ, Rᵤ and Rᵥ is optionally further substituted by one or more substituents selected from halo, trifluoromethyl, trifluoromethoxy, cyano, hydroxyl, carboxyl, carbamoyl, sulphamoyl, and (1-2C)alkyl; and
(ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;

18. A compound according to any one of the preceding claims, or a pharmaceutically acceptable salt or solvate thereof, wherein R₃ is selected from hydrogen, cyano, (1-8C)alkyl, (3-7C)cycloalkyl, —(CH₂)₁₋₃(3-7C)cycloalkyl, a carbon-linked 4 to 7 membered heterocyclyl, a carbon-linked 5 to 6 membered heteroaryl, —C(O)—(1-8C)alkyl, —C(O)(3-7C)cycloalkyl, —C(O)phenyl, —C(O)O(1-8C)alkyl, —C(O)NH₂, —C(O)NH—(1-8C)alkyl, —C(O)NH—(CH₂)₀₋₃(3-7C)cycloalkyl, —C(O)NH—(CH₂)₀₋₃[5 or 6-membered heteroaryl], —C(O)NH—(CH₂)₀₋₃phenyl, —S(O)₂H or —S(O)₂-(1-6C)alkyl;
wherein any alkyl, cycloalkyl, phenyl, or heteroaryl moiety is optionally substituted by one or more R₃₀₀ substituents;
wherein R₃₀₀ is selected from halo, trifluoromethyl, trifluoromethoxy, cyano, hydroxyl, (1-4C)alkyl, (1-4C)hydroxyalkyl, ORₛ, C(O)N(Rᵥ)Rᵤ, S(O)_{y}Rₛ, (CH₂)_{z}NRᵤRᵥ;
and wherein:
(i) Rₛ and Rₜ are each independently selected from hydrogen, (1-6C)alkyl or (CH₂)_{z}phenyl; Rᵤ and Rᵥ are each independently selected from hydrogen, (1-6C)alkyl or (CH₂)_{z}phenyl or Rᵤ and Rᵥ, together with the nitrogen atom to which they are attached, form a 3-7 membered ring which may optionally include further heteroatoms; and
(ii) any cycloalkyl, heterocyclyl, heteroaryl or phenyl moiety in a R₃₀₀ substituent group is optionally further substituted by one or more substituents selected from halo, trifluoromethyl, trifluoromethoxy, cyano, hydroxyl, or (1-2C)alkyl;

19. A compound according to any one of the preceding claims, or a pharmaceutically acceptable salt or solvate thereof, wherein R₃ is selected from hydrogen or acetyl.

20. A compound according to any one of the preceding claims, or a pharmaceutically acceptable salt or solvate thereof, wherein R₄ is selected from hydrogen or fluoro.

21. A compound according to any one of the preceding claims, or a pharmaceutically acceptable salt or solvate thereof, wherein R₅ is selected from hydrogen, cyano or fluoro.

22. A compound according to any one of the preceding claims, or a pharmaceutically acceptable salt or solvate thereof, wherein Q is hydrogen, or a group of the formula:
-L₁-Y₁-L₂-Q₁
wherein:
L₁ is absent or (1-3C)alkylene;
Y₁ is absent or O, S, SO, SO₂, N(R_{y1}), C(O), C(O)O, C(O)N(R_{y1}), or N(R_{y1})C(O), wherein R_{y1} is selected from hydrogen or (1-4C)alkyl;
L₂ is absent or (1-3C)alkylene; and
Q₁ is hydrogen, (1-6C)alkyl, phenyl, (3-8C)cycloalkyl, heteroaryl or heterocyclyl;
wherein Q is optionally further substituted by one or more substituent groups independently selected from oxo, hydroxy, (1-6C)alkyl, halo, (1-4C)haloalkyl, (1-4C)haloalkoxy, (1-4C)aminoalkyl, (1-4C)hydroxyalkyl, cyano, or by one or more group(s) of the formula:
-L₃-Y₂-L₄-W₁
wherein:
L₃ is absent;
Y₂ is absent or selected from or O, S, SO, SO₂, N(R_{y2}), C(O), C(O)O, OC(O), C(O)N(R_{y2}), or N(R_{y2})C(O), S(O)₂N(R_{y2}), N(R_{y2})SO₂ wherein R_{y2} is selected from hydrogen or (1-3C)alkyl;
L₄ is absent or (1-3C)alkylene; and
W₁ is hydrogen, (1-6C)alkyl, or phenyl;
wherein W₁ is optionally substituted by one or more substituents selected from (1-2C)alkyl, or halo;
with the proviso that Q₁ is not aryl or heteroaryl when L₁, Y₁ and L₂ are absent.

23. A compound according to any preceding claim, or a pharmaceutically acceptable salt or solvate thereof, selected from
5-(2-aminopyridin-4-yl)-7-chloro-1H-indazol-3-amine;
5-(2-aminopyridin-4-yl)-7-methyl-1H-indazol-3-amine;
5-(2-aminopyridin-4-yl)-7-(trifluoromethyl)-1H-indazol-3-amine;
7-chloro-5-(1H-pyrrolo[2,3-b]pyridin-4-yl)-1H-indazol-3-amine;
7-bromo-5-(1H-pyrrolo[2,3-b]pyridin-4-yl)-1H-indazol-3-amine;
7-ethynyl-5-(1H-pyrrolo[2,3-b]pyridin-4-yl)-1H-indazol-3-amine;
7-phenyl-5-(1H-pyrrolo[2,3-b]pyridin-4-yl)-1H-indazol-3-amine;
5-(2-methyl-1H-pyrrolo[2,3-b]pyridin-4-yl)-1H-indazol-3-amine;
5-(2-(tert-butyl)-1H-pyrrolo[2,3-b]pyridin-4-yl)-1H-indazol-3-amine;
4-(3-amino-1H-indazol-5-yl)-1H-pyrrolo[2,3-b]pyridine-2-carboxylic acid;
7-bromo-5-(7H-pyrrolo[2,3-d]pyrimidin-4-yl)-1H-indazol-3-amine 5-(2-(ethylamino)pyridin-4-yl)-1H-indazol-3-amine;
5-(2-(propylamino)pyridin-4-yl)-1H-indazol-3-amine;
5-(2-(isopropylamino)pyridin-4-yl)-1H-indazol-3-amine;
5-(2-((cyclopropylmethyl)amino)pyridin-4-yl)-1H-indazol-3-amine;
5-(2-(isopentylamino)pyridin-4-yl)-1H-indazol-3-amine;
5-(2-(hexylamino)pyridin-4-yl)-1H-indazol-3-amine;
5-(2-(cyclohexylamino)pyridin-4-yl)-1H-indazol-3-amine;
5-{2-[(Trans-4-methylcyclohexyl)amino]pyridin-4-yl}-1H-indazol-3-amine;
2-((4-(3-amino-1H-indazol-5-yl)pyridin-2-yl)amino)ethan-1-ol;
3-((4-(3-amino-1H-indazol-5-yl)pyridin-2-yl)amino)propan-1-ol;
4-((4-(3-amino-1H-indazol-5-yl)pyridin-2-yl)amino)butan-1-ol;
5-((4-(3-amino-1H-indazol-5-yl)pyridin-2-yl)amino)pentan-1-ol;
5-{2-[(trans-4-hydroxycyclohexyl)amino]pyridin-4-yl}-1H-indazol-3-amine;
5-(2-((2-methoxyethyl)amino)pyridin-4-yl)-1H-indazol-3-amine;
5-(2-((3-methoxypropyl)amino)pyridin-4-yl)-1H-indazol-3-amine;
5-(2-((3-isopropoxypropyl)amino)pyridin-4-yl)-1H-indazol-3-amine;
3-((4-(3-amino-1H-indazol-5-yl)pyrimidin-2-yl)amino)propan-1-ol;
3-((4-(3-amino-1H-indazol-5-yl)pyridin-2-yl)(methyl)amino)propan-1-ol;
5-(2-((2-morpholinoethyl)amino)pyridin-4-yl)-1H-indazol-3-amine;
5-(2-((2-(piperidin-1-yl)ethyl)amino)pyridin-4-yl)-1H-indazol-3-amine;
N¹-(4-(3-amino-1H-indazol-5-yl)pyridin-2-yl)-N3-methylpropane-1,3-diamine;
N-(4-(3-amino-1H-indazol-5-yl)pyridin-2-yl)cyclopropanecarboxamide;
N-(4-(3-amino-1H-indazol-5-yl)pyridin-2-yl)benzamide;
ethyl (4-(3-amino-1H-indazol-5-yl)pyridine-2-yl)carbamate;
1-(4-(3-amino-1H-indazol-5-yl)pyridine-2-yl)-3-ethylurea;
1-(4-(3-amino-1H-indazol-5-yl)pyridine-2-yl)-3-ethylurea;
1-(4-(3-amino-1H-indazol-5-yl)pyridine-2-yl)-3-propylurea;
1-(4-(3-amino-1H-indazol-5-yl)pyridine-2-yl)-3-isopentylurea;
1-(4-(3-amino-1H-indazol-5-yl)pyridine-2-yl)-3-cyclopentylurea;
1-(4-(3-amino-1H-indazol-5-yl)pyridine-2-yl)-3-cyclohexylurea;
1-(4-(3-amino-1H-indazol-5-yl)pyridine-2-yl)-3-(2-hydroxyethyl)urea;
1-(4-(3-amino-1H-indazol-5-yl)pyridine-2-yl)-3-(3-hydroxypropyl)urea;
1-(4-(3-amino-1H-indazol-5-yl)pyridine-2-yl)-3-(2-methoxyethyl)urea;
3-(4-(3-amino-1H-indazol-5-yl)pyridine-2-yl)-1-(2-hydroxyethyl)-1-methylurea;
1-(4-(3-amino-1H-indazol-5-yl)pyridine-2-yl)-3-benzylurea;
1-(4-(3-amino-1H-indazol-5-yl)pyridine-2-yl)-3-phenethylurea;
1-(4-(3-amino-1H-indazol-5-yl)pyridine-2-yl)-3-(pyridine-2-ylmethyl)urea;
1-(4-(3-amino-1H-indazol-5-yl)pyridine-2-yl)-3-(pyridine-3-ylmethyl)urea;
1-(4-(3-amino-1H-indazol-5-yl)pyridine-2-yl)-3-(pyridine-4-ylmethyl)urea;
1-(4-(3-amino-1H-indazol-5-yl)pyridine-2-yl)-3-phenylurea;
1-(4-(3-amino-1H-indazol-5-yl)pyridine-2-yl)-3-(3-fluorophenyl)urea;
1-(4-(3-amino-1H-indazol-5-yl)pyridine-2-yl)-3-(3-chlorophenyl)urea;
1-(4-(3-amino-1H-indazol-5-yl)pyridine-2-yl)-3-(3-isopropylphenyl)urea;
1-(4-(3-amino-1H-indazol-5-yl)pyridine-2-yl)-3-(3-(hydroxymethyl)phenyl)urea;
3-(3-(4-(3-amino-1H-indazol-5-yl)pyridine-2-yl)ureido)benzamide;
1-(4-(3-amino-1H-indazol-5-yl)pyridine-2-yl)-3-(3-phenoxyphenyl)urea;
1-(4-(3-amino-1H-indazol-5-yl)pyridine-2-yl)-3-(3-(benzyloxy)phenyl)urea;
1-(4-(3-amino-1H-indazol-5-yl)pyridine-2-yl)-3-(3-((4-fluorobenzyl)oxy)phenyl)urea;
1-(4-(3-amino-1H-indazol-5-yl)pyridine-2-yl)-3-(3-((3-fluorobenzyl)oxy)phenyl)urea;
1-(4-(3-amino-1H-indazol-5-yl)pyridine-2-yl)-3-(3-((2-fluorobenzyl)oxy)phenyl)urea;
3-(3-(4-(3-amino-1H-indazol-5-yl)pyridine-2-yl)ureido)-N-phenylbenzamide;
1-(4-(3-amino-1H-indazol-5-yl)pyridine-2-yl)-3-(4-fluorophenyl)urea;
1-(4-(3-amino-1H-indazol-5-yl)pyridine-2-yl)-3-(4-chlorophenyl)urea;
1-(4-(3-amino-1H-indazol-5-yl)pyridine-2-yl)-3-(4-(tert-butyl)phenyl)urea;
1-(4-(3-amino-1H-indazol-5-yl)pyridine-2-yl)-3-(4-(methylsulfonyl)phenyl)urea;
1-(4-(3-amino-1H-indazol-5-yl)pyridin-2-yl)-3-(o-tolyl)urea;
1-(4-(3-amino-1H-indazol-5-yl)pyridine-2-yl)-3-(2-ethylphenyl)urea;
1-(4-(3-amino-1H-indazol-5-yl)pyridine-2-yl)-3-(2-isopropylphenyl)urea;
1-(4-(3-amino-1H-indazol-5-yl)pyridine-2-yl)-3-(pyridine-3-yl)urea;
5-(2-amino-3-ethynylpyridin-4-yl)-1H-indazol-3-amine;
5-(2-amino-3-(cyclopropylethynyl)pyridin-4-yl)-1H-indazol-3-amine;
5-(2-amino-3-(3,3-dimethylbut-1-yn-1-yl)pyridine-4-yl)-1H-indazol-3-amine;
5-(2-amino-3-(cyclopentylethynyl)pyridine-4-yl)-1H-indazol-3-amine;
5-(2-amino-3-(cyclohexylethynyl)pyridine-4-yl)-1H-indazol-3-amine;
5-(2-amino-3-(phenylethynyl)pyridine-4-yl)-1H-indazol-3-amine;
5-(2-amino-3-((4-aminophenyl)ethynyl)pyridine-4-yl)-1H-indazol-3-amine;
5-(2-amino-3-((3-aminophenyl)ethynyl)pyridine-4-yl)-1H-indazol-3-amine;
5-(2-amino-3-((2-aminophenyl)ethynyl)pyridine-4-yl)-1H-indazol-3-amine;
methyl 3-((2-amino-4-(3-amino-1H-indazol-5-yl)pyridine-3-yl)ethynyl)benzoate;
methyl 4-((2-amino-4-(3-amino-1H-indazol-5-yl)pyridine-3-yl)ethynyl)benzoate;
5-(2-amino-3-((2-methoxyphenyl)ethynyl)pyridine-4-yl)-1H-indazol-3-amine;
5-(2-amino-4-(3-amino-1H-indazol-5-yl)pyridine-3-yl)-1-phenylpent-4-yn-1-one;
3-(2-amino-4-(3-amino-1H-indazol-5-yl)pyridine-3-yl)prop-2-yn-1-ol;
4-(2-amino-4-(3-amino-1H-indazol-5-yl)pyridine-3-yl)but-3-yn-1-ol;
5-(2-amino-4-(3-amino-1H-indazol-5-yl)pyridine-3-yl)pent-4-yn-1-ol;
6-(2-amino-4-(3-amino-1H-indazol-5-yl)pyridine-3-yl)-2-methylhex-5-yn-2-ol;
4-(2-amino-4-(3-amino-1H-indazol-5-yl)pyridine-3-yl)-2-methylbut-3-yn-2-ol;
5-(2-amino-3-(3-(tert-butoxy)prop-1-yn-1-yl)pyridine-4-yl)-1H-indazol-3-amine;
1-((2-amino-4-(3-amino-1H-indazol-5-yl)pyridine-3-yl)ethynyl)cyclopentan-1-ol;
1-((2-amino-4-(3-amino-1H-indazol-5-yl)pyridine-3-yl)ethynyl)cyclohexan-1-ol;
1-((2-amino-4-(3-amino-1H-indazol-5-yl)pyridine-3-yl)ethynyl)cycloheptan-1-ol;
5-(2-amino-3-(3-amino-3-methylbut-1-yn-1-yl)pyridin-4-yl)-1H-indazol-3-amine;
5-(2-amino-3-(4-(piperidin-1-yl)but-1-yn-1-yl)pyridin-4-yl)-1H-indazol-3-amine;
5-(2-amino-3-(4-morpholinobut-1-yn-1-yl)pyridin-4-yl)-1H-indazol-3-amine;
5-(2-amino-3-(5-(piperidin-1-yl)pent-1-yn-1-yl)pyridin-4-yl)-1H-indazol-3-amine;
5-(2-amino-3-(5-morpholinopent-1-yn-1-yl)pyridin-4-yl)-1H-indazol-3-amine;
5-(2-amino-3-(3-(piperidin-4-yl)prop-1-yn-1-yl)pyridin-4-yl)-1H-indazol-3-amine;
3-(2-amino-4-(3-amino-1H-indazol-5-yl)pyridin-3-yl)-N-methylpropiolamide;
5-(2-amino-4-(3-amino-1H-indazol-5-yl)pyridine-3-yl)-1-morpholinopent-4-yn-1-one;
5-(2-amino-3-cyclopropylpyridin-4-yl)-1H-indazol-3-amine;
4-(2-amino-4-(3-amino-1H-indazol-5-yl)pyridin-3-yl)butan-1-ol;
1-(2-(2-amino-4-(3-amino-1H-indazol-5-yl)pyridin-3-yl)ethyl)cyclohexan-1-ol;
5-(2-amino-4-(3-amino-1H-indazol-5-yl)pyridin-3-yl)pentan-1-ol;
5-(2-aminopyridin-4-yl)-7-phenyl-1H-indazol-3-amine;
5-(2-aminopyridin-4-yl)-7-(3-fluorophenyl)-1H-indazol-3-amine;
5-(2-aminopyridin-4-yl)-7-(3-(trifluoromethyl)phenyl)-1H-indazol-3-amine;
7-(3-aminophenyl)-5-(2-aminopyridin-4-yl)-1H-indazol-3-amine;
3-(3-amino-5-(2-aminopyridin-4-yl)-1H-indazol-7-yl) phenol;
5-(2-aminopyridin-4-yl)-7-(3-methoxyphenyl)-1H-indazol-3-amine;
(3-(3-amino-5-(2-aminopyridin-4-yl)-1H-indazol-7-yl)phenyl)methanol;
3-(3-amino-5-(2-aminopyridin-4-yl)-1H-indazol-7-yl)benzaldehyde;
ethyl 3-(3-amino-5-(2-aminopyridin-4-yl)-1H-indazol-7-yl)benzoate;
3-(3-amino-5-(2-aminopyridin-4-yl)-1H-indazol-7-yl)benzamide;
3-(3-amino-5-(2-aminopyridin-4-yl)-1H-indazol-7-yl)benzenesulfonamide;
5-(2-aminopyridin-4-yl)-7-(3-(methylsulfonyl)phenyl)-1H-indazol-3-amine;
5-(2-aminopyridin-4-yl)-7-(3-(morpholinomethyl)phenyl)-1H-indazol-3-amine;
4-(3-amino-5-(2-aminopyridin-4-yl)-1H-indazol-7-yl) phenol;
(4-(3-amino-5-(2-aminopyridin-4-yl)-1H-indazol-7-yl)phenyl)methanol;
5-(2-aminopyridin-4-yl)-7-(4-(dimethylamino)phenyl)-1H-indazol-3-amine;
4-(3-amino-5-(2-aminopyridin-4-yl)-1H-indazol-7-yl)benzamide;
4-(3-amino-5-(2-aminopyridin-4-yl)-1H-indazol-7-yl)benzenesulfonamide;
5-(2-aminopyridin-4-yl)-7-(4-(morpholinomethyl)phenyl)-1H-indazol-3-amine;
5-(2-aminopyridin-4-yl)-7-(4-(tert-butyl)phenyl)-1H-indazol-3-amine;
5-(2-aminopyridin-4-yl)-7-(2-chlorophenyl)-1H-indazol-3-amine;
(2-(3-amino-5-(2-aminopyridin-4-yl)-1H-indazol-7-yl)phenyl)methanol;
4-(3-amino-5-(2-aminopyridin-4-yl)-1H-indazol-7-yl)-3-methylbenzenesulfonamide;
5-(2-aminopyridin-4-yl)-7-(pyridin-3-yl)-1H-indazol-3-amine;
5-(2-aminopyridin-4-yl)-7-(pyridin-4-yl)-1H-indazol-3-amine;
5-(2-aminopyridin-4-yl)-7-(furan-3-yl)-1H-indazol-3-amine;
5-(2-aminopyridin-4-yl)-7-(thiophen-3-yl)-1H-indazol-3-amine;
5-(2-aminopyridin-4-yl)-7-(thiophen-2-yl)-1H-indazol-3-amine;
5-(2-aminopyridin-4-yl)-7-(thiazol-5-yl)-1H-indazol-3-amine;
5-(2-aminopyridin-4-yl)-7-(1H-pyrazol-5-yl)-1H-indazol-3-amine;
5-(2-aminopyridin-4-yl)-7-(3-methylbut-1-yn-1-yl)-1H-indazol-3-amine;
5-(2-aminopyridin-4-yl)-7-(pent-1-yn-1-yl)-1H-indazol-3-amine;
5-(2-aminopyridin-4-yl)-7-(cyclopropylethynyl)-1H-indazol-3-amine;
5-(2-aminopyridin-4-yl)-7-(3,3-dimethylbut-1-yn-1-yl)-1H-indazol-3-amine;
5-(2-aminopyridin-4-yl)-7-(phenylethynyl)-1H-indazol-3-amine;
4-(3-amino-5-(2-aminopyridin-4-yl)-1H-indazol-7-yl)but-3-yn-1-ol;
4-(3-amino-5-(2-aminopyridin-4-yl)-1H-indazol-7-yl)-2-methylbut-3-yn-2-ol;
5-(2-aminopyridin-4-yl)-7-((3-methyloxetan-3-yl)ethynyl)-1H-indazol-3-amine;
5-(2-aminopyridin-4-yl)-7-((tetrahydro-2H-pyran-4-yl)ethynyl)-1H-indazol-3-amine;
5-(2-aminopyrimidin-4-yl)-7-(3,3-dimethylbut-1-yn-1-yl)-1H-indazol-3-amine;
5-(2-aminopyridin-4-yl)-7-(3,3-dimethylbutyl)-1H-indazol-3-amine;
5-(2-aminopyridin-4-yl)-7-(2-cyclohexylethyl)-1H-indazol-3-amine;
5-(2-aminopyridin-4-yl)-7-(2-cyclopropylethyl)-1H-indazol-3-amine;
5-(2-aminopyridin-4-yl)-7-phenethyl-1H-indazol-3-amine;
5-(2-amino-5-fluoropyridin-4-yl)-7-(3,3-dimethylbut-1-yn-1-yl)-1H-indazol-3-amine;
5-(2-amino-3-fluoropyridin-4-yl)-7-(3,3-dimethylbut-1-yn-1-yl)-1H-indazol-3-amine;
5-(2-amino-6-fluoropyridin-4-yl)-7-(3,3-dimethylbut-1-yn-1-yl)-1H-indazol-3-amine;
4-(3-amino-7-(3,3-dimethylbut-1-yn-1-yl)-1H-indazol-5-yl)pyridine-2,6-diamine;
6-amino-4-(3-amino-7-(3,3-dimethylbut-1-yn-1-yl)-1H-indazol-5-yl)nicotinonitrile;
5-(2-(cyclopropylamino)pyridine-4-yl)-7-(3,3-dimethylbut-1-yn-1-yl)-1H-indazol-3-amine;
5-(2-(cyclobutylamino)pyridin-4-yl)-7-(3,3-dimethylbut-1-yn-1-yl)-1H-indazol-3-amine;
7-(3,3-dimethylbut-1-yn-1-yl)-5-(2-(oxetan-3-ylamino)pyridin-4-yl)-1H-indazol-3-amine;
5-(2-(cyclopentylamino)pyridine-4-yl)-7-(3,3-dimethylbut-1-yn-1-yl)-1H-indazol-3-amine;
5-(2-((cyclopropylmethyl)amino)pyridin-4-yl)-7-(3,3-dimethylbut-1-yn-1-yl)-1H-indazol-3-amine;
7-(3,3-dimethylbut-1-yn-1-yl)-5-(2-((2,2,2-trifluoroethyl)amino)pyridin-4-yl)-1H-indazol-3-amine;
3-((4-(3-amino-7-(3,3-dimethylbut-1-yn-1-yl)-1H-indazol-5-yl)pyridin-2-yl)amino)propanenitrile;
2-((4-(3-amino-7-(3,3-dimethylbut-1-yn-1-yl)-1H-indazol-5-yl)pyridin-2-yl)amino)ethan-1-ol;
N1-(4-(3-amino-7-(3,3-dimethylbut-1-yn-1-yl)-1H-indazol-5-yl)pyridin-2-yl)ethane-1,2-diamine;
7-(3,3-dimethylbut-1-yn-1-yl)-5-(2-((2-methoxyethyl)amino)pyridin-4-yl)-1H-indazol-3-amine;
7-(3,3-dimethylbut-1-yn-1-yl)-5-(2-((3-methoxypropyl)amino)pyridine-4-yl)-1H-indazol-3-amine;
N-(4-(3-amino-7-(3,3-dimethylbut-1-yn-1-yl)-1H-indazol-5-yl)pyridin-2-yl)acetamide;
N-(4-(3-amino-7-(3,3-dimethylbut-1-yn-1-yl)-1H-indazol-5-yl)pyridin-2-yl)propionamide;
N-(4-(3-amino-7-(3,3-dimethylbut-1-yn-1-yl)-1H-indazol-5-yl)pyridin-2-yl)-3,3,3-trifluoropropanamide;
N-(4-(3-amino-7-(3,3-dimethylbut-1-yn-1-yl)-1H-indazol-5-yl)pyridin-2-yl)cyclopropanecarboxamide;
N-(4-(3-amino-7-(3,3-dimethylbut-1-yn-1-yl)-1H-indazol-5-yl)pyridin-2-yl)isobutyramide;
N-(4-(3-amino-7-(3,3-dimethylbut-1-yn-1-yl)-1H-indazol-5-yl)pyridin-2-yl)pivalamide;
N-(4-(3-amino-7-(3,3-dimethylbut-1-yn-1-yl)-1H-indazol-5-yl)pyridin-2-yl)-2-cyclopropylacetamide;
N-(4-(3-amino-7-(3,3-dimethylbut-1-yn-1-yl)-1H-indazol-5-yl)pyridine-2-yl)-3-methylbutanamide;
N-(4-(3-amino-7-(3,3-dimethylbut-1-yn-1-yl)-1H-indazol-5-yl)pyridin-2-yl)cyclobutanecarboxamide;
N-(4-(3-amino-7-(3,3-dimethylbut-1-yn-1-yl)-1H-indazol-5-yl)pyridin-2-yl)cyclopentanecarboxamide;
N-(4-(3-amino-7-(3,3-dimethylbut-1-yn-1-yl)-1H-indazol-5-yl)pyridin-2-yl)-2-hydroxyacetamide;
N-(4-(3-amino-7-(3,3-dimethylbut-1-yn-1-yl)-1H-indazol-5-yl)pyridin-2-yl)-2-methoxyacetamide;
5-(2-(cyclopropylamino)pyridine-4-yl)-7-((3-methyloxetan-3-yl)ethynyl)-1H-indazol-3-amine;
N-(4-(3-amino-7-((3-methyloxetan-3-yl)ethynyl)-1H-indazol-5-yl)pyridin-2-yl)acetamide;
N-(4-(3-amino-7-(phenylethynyl)-1H-indazol-5-yl)pyridin-2-yl)acetamide;
methyl (4-(3-amino-7-(cyclopropylethynyl)-1H-indazol-5-yl)pyridin-2-yl)carbamate;
methyl (4-(3-amino-7-(3-hydroxy-3-methylbut-1-yn-1-yl)-1H-indazol-5-yl)pyridin-2-yl)carbamate;
methyl (4-(3-amino-7-(3-amino-3-methylbut-1-yn-1-yl)-1H-indazol-5-yl)pyridin-2-yl)carbamate;
methyl (4-(3-amino-7-(3-methoxy-3-methylbut-1-yn-1-yl)-1H-indazol-5-yl)pyridin-2-yl)carbamate;
methyl (4-(3-amino-7-(3-morpholinoprop-1-yn-1-yl)-1H-indazol-5-yl)pyridin-2-yl)carbamate;
Methyl (4-(3-amino-7-(4-morpholinobut-1-yn-1-yl)-1H-indazol-5-yl)pyridin-2-yl)carbamate;
1-(4-(3-amino-7-(3,3-dimethylbut-1-yn-1-yl)-1H-indazol-5-yl)pyridin-2-yl)urea;
1-(4-(3-amino-7-(3,3-dimethylbut-1-yn-1-yl)-1H-indazol-5-yl)pyridin-2-yl)-3-methylurea;
1-(4-(3-amino-7-(3,3-dimethylbut-1-yn-1-yl)-1H-indazol-5-yl)pyridin-2-yl)-3-ethylurea;
1-(4-(3-amino-7-(3,3-dimethylbut-1-yn-1-yl)-1H-indazol-5-yl)pyridin-2-yl)-3-propylurea;
1-(4-(3-amino-7-(3,3-dimethylbut-1-yn-1-yl)-1H-indazol-5-yl)pyridine-2-yl)-3-phenylurea;
methyl (4-(3-amino-7-(3,3-dimethylbut-1-yn-1-yl)-1H-indazol-5-yl)pyridin-2-yl)carbamate;
ethyl (4-(3-amino-7-(3,3-dimethylbut-1-yn-1-yl)-1H-indazol-5-yl)pyridin-2-yl)carbamate;
tert-butyl (4-(3-amino-7-(3,3-dimethylbut-1-yn-1-yl)-1H-indazol-5-yl)pyridin-2-yl)carbamate;
(4-(3-amino-7-(3,3-dimethylbut-1-yn-1-yl)-1H-indazol-5-yl)pyridin-2-yl)sulfamic acid;
N-(4-(3-amino-7-(3,3-dimethylbut-1-yn-1-yl)-1H-indazol-5-yl)pyridin-2-yl)methanesulfonamide;
N-(4-(3-amino-7-(3,3-dimethylbut-1-yn-1-yl)-1H-indazol-5-yl)-6-fluoropyridin-2-yl)acetamide;
N-(4-(3-amino-7-phenyl-1H-indazol-5-yl)pyridin-2-yl)acetamide;
N-(4-(3-amino-7-(pyridin-4-yl)-1H-indazol-5-yl)pyridin-2-yl)acetamide;
7-(furan-3-yl)-5-(1H-pyrrolo[2,3-b]pyridin-4-yl)-1H-indazol-3-amine;
7-ethynyl-5-(1H-pyrrolo[2,3-b]pyridin-4-yl)-1H-indazol-3-amine;
7-(3,3-dimethylbut-1-yn-1-yl)-5-(1H-pyrrolo[2,3-b]pyridin-4-yl)-1H-indazol-3-amine;
7-(cyclopropylethynyl)-5-(1H-pyrrolo[2,3-b]pyridin-4-yl)-1H-indazol-3-amine;
7-(cyclopentylethynyl)-5-(1H-pyrrolo[2,3-b]pyridin-4-yl)-1H-indazol-3-amine;
7-(cyclohexylethynyl)-5-(1H-pyrrolo[2,3-b]pyridin-4-yl)-1H-indazol-3-amine;
3-(3-amino-5-(1H-pyrrolo[2,3-b]pyridin-4-yl)-1H-indazol-7-yl)prop-2-yn-1-ol;
4-(3-amino-5-(1H-pyrrolo[2,3-b]pyridin-4-yl)-1H-indazol-7-yl)-2-methylbut-3-yn-2-ol;
1-((3-amino-5-(1H-pyrrolo[2,3-b]pyridin-4-yl)-1H-indazol-7-yl)ethynyl)cyclopentan-1-ol;
1-((3-amino-5-(1H-pyrrolo[2,3-b]pyridin-4-yl)-1H-indazol-7-yl)ethynyl)cyclohexan-1-ol;
1-((3-amino-5-(1H-pyrrolo[2,3-b]pyridin-4-yl)-1H-indazol-7-yl)ethynyl)cycloheptan-1-ol;
7-(5-morpholinopent-1-yn-1-yl)-5-(1H-pyrrolo[2,3-b]pyridin-4-yl)-1H-indazol-3-amine;
7-(4-(piperidin-1-yl)but-1-yn-1-yl)-5-(1H-pyrrolo[2,3-b]pyridin-4-yl)-1H-indazol-3-amine;
7-(5-(piperidin-1-yl)pent-1-yn-1-yl)-5-(1H-pyrrolo[2,3-b]pyridin-4-yl)-1H-indazol-3-amine;
7-(6-(piperidin-1-yl)hex-1-yn-1-yl)-5-(1H-pyrrolo[2,3-b]pyridin-4-yl)-1H-indazol-3-amine;
6-(3-amino-5-(1H-pyrrolo[2,3-b]pyridin-4-yl)-1H-indazol-7-yl)hex-5-ynoic acid;
7-(3-amino-5-(1H-pyrrolo[2,3-b]pyridin-4-yl)-1H-indazol-7-yl)hept-6-ynoic acid;
7-(4-phenoxybut-1-yn-1-yl)-5-(1H-pyrrolo[2,3-b]pyridin-4-yl)-1H-indazol-3-amine;
7-(6-phenoxyhex-1-yn-1-yl)-5-(1H-pyrrolo[2,3-b]pyridin-4-yl)-1H-indazol-3-amine;
5-(2-methyl-1H-pyrrolo[2,3-b]pyridin-4-yl)-1H-indazol-3-amine;
5-(2-(tert-butyl)-1H-pyrrolo[2,3-b]pyridin-4-yl)-1H-indazol-3-amine;
5-(2-cyclopropyl-1H-pyrrolo[2,3-b]pyridin-4-yl)-1H-indazol-3-amine;
5-(2-cyclohexyl-1H-pyrrolo[2,3-b]pyridin-4-yl)-1H-indazol-3-amine;
5-(2-neopentyl-1H-pyrrolo[2,3-b]pyridin-4-yl)-1H-indazol-3-amine;
5-(2-(cyclohexylmethyl)-1H-pyrrolo[2,3-b]pyridin-4-yl)-1H-indazol-3-amine;
5-(2-(2-cyclohexylethyl)-1H-pyrrolo[2,3-b]pyridin-4-yl)-1H-indazol-3-amine;
5-(2-benzyl-1H-pyrrolo[2,3-b]pyridin-4-yl)-1H-indazol-3-amine;
(4-(3-amino-1H-indazol-5-yl)-1H-pyrrolo[2,3-b]pyridin-2-yl)methanol;
2-(4-(3-amino-1H-indazol-5-yl)-1H-pyrrolo[2,3-b]pyridin-2-yl)propan-2-ol;
3-(4-(3-amino-1H-indazol-5-yl)-1H-pyrrolo[2,3-b]pyridin-2-yl)pentan-3-ol;
5-(2-(tert-butoxymethyl)-1H-pyrrolo[2,3-b]pyridin-4-yl)-1H-indazol-3-amine;
5-(2-(tetrahydro-2H-pyran-4-yl)-1H-pyrrolo[2,3-b]pyridin-4-yl)-1H-indazol-3-amine;
5-(2-(tetrahydro-2H-pyran-2-yl)-1H-pyrrolo[2,3-b]pyridin-4-yl)-1H-indazol-3-amine;
5-(2-((tetrahydro-2H-pyran-4-yl)methyl)-1H-pyrrolo[2,3-b]pyridin-4-yl)-1H-indazol-3-amine;
4-(3-amino-1H-indazol-5-yl)-1H-pyrrolo[2,3-b]pyridine-2-carboxylic acid;
methyl 4-(3-amino-1H-indazol-5-yl)-1H-pyrrolo[2,3-b]pyridine-2-carboxylate;
ethyl 4-(3-amino-1H-indazol-5-yl)-1H-pyrrolo[2,3-b]pyridine-2-carboxylate;
4-(3-amino-1H-indazol-5-yl)-1H-pyrrolo[2,3-b]pyridine-2-carboxamide;
(4-(3-amino-1H-indazol-5-yl)-1H-pyrrolo[2,3-b]pyridin-2-yl)(pyrrolidin-1-yl)methanone;
4-(3-amino-1H-indazol-5-yl)-N-cyclopentyl-1H-pyrrolo[2,3-b]pyridine-2-carboxamide;
4-(3-amino-1H-indazol-5-yl)-N-cyclohexyl-1H-pyrrolo[2,3-b]pyridine-2-carboxamide;
4-(3-amino-1H-indazol-5-yl)-N-isopentyl-1H-pyrrolo[2,3-b]pyridine-2-carboxamide;
4-(3-amino-1H-indazol-5-yl)-N-phenethyl-1H-pyrrolo[2,3-b]pyridine-2-carboxamide;
4-(3-amino-1H-indazol-5-yl)-N-(3-phenylpropyl)-1H-pyrrolo[2,3-b]pyridine-2-carboxamide;
4-(3-amino-1H-indazol-5-yl)-N-(2-methoxyethyl)-1H-pyrrolo[2,3-b]pyridine-2-carboxamide;
4-(3-amino-1H-indazol-5-yl)-N-(2-aminoethyl)-1H-pyrrolo[2,3-b]pyridine-2-carboxamide;
4-(3-amino-1H-indazol-5-yl)-N-(2-(dimethylamino)ethyl)-1H-pyrrolo[2,3-b]pyridine-2-carboxamide;
(4-(3-amino-1H-indazol-5-yl)-1H-pyrrolo[2,3-b]pyridin-2-yl)(4-methylpiperazin-1-yl)methanone;
4-(3-amino-1H-indazol-5-yl)-N-(2-(piperidin-1-yl)ethyl)-1H-pyrrolo[2,3-b]pyridine-2-carboxamide 4-(3-amino-1H-indazol-5-yl)-N-(2-(butyl(ethyl)amino)ethyl)-1H-pyrrolo[2,3-b]pyridine-2-carboxamide;
4-(3-amino-1H-indazol-5-yl)-N-(2-(diisopropylamino)ethyl)-1H-pyrrolo[2,3-b]pyridine-2-carboxamide;
4-(3-amino-1H-indazol-5-yl)-N-(3-(dimethylamino)propyl)-1H-pyrrolo[2,3-b]pyridine-2-carboxamide;
5-(2-((tert-butylamino)methyl)-1H-pyrrolo[2,3-b]pyridine-4-yl)-1H-indazol-3-amine 5-(2-((isopentylamino)methyl)-1H-pyrrolo[2,3-b]pyridine-4-yl)-1H-indazol-3-amine;
5-(2-(piperidin-2-yl)-1H-pyrrolo[2,3-b]pyridine-4-yl)-1H-indazol-3-amine;
5-(2-((cyclohexylamino)methyl)-1H-pyrrolo[2,3-b]pyridine-4-yl)-1H-indazol-3-amine;
5-(2-((phenylamino)methyl)-1H-pyrrolo[2,3-b]pyridine-4-yl)-1H-indazol-3-amine;
5-(2-(((2-(benzyloxy)phenyl)amino)methyl)-1H-pyrrolo[2,3-b]pyridine-4-yl)-1H-indazol-3-amine;
5-(2-(((2-methoxyethyl)amino)methyl)-1H-pyrrolo[2,3-b]pyridine-4-yl)-1H-indazol-3-amine;
N¹-((4-(3-amino-1H-indazol-5-yl)-1H-pyrrolo[2,3-b]pyridin-2-yl)methyl)-N²,N²-dimethylethane-1,2-diamine;
5-(2-(((3-methoxypropyl)amino)methyl)-1H-pyrrolo[2,3-b]pyridine-4-yl)-1H-indazol-3-amine;
5-(2-(((3-isopropoxypropyl)amino)methyl)-1H-pyrrolo[2,3-b]pyridine-4-yl)-1H-indazol-3-amine;
N¹-((4-(3-amino-1H-indazol-5-yl)-1H-pyrrolo[2,3-b]pyridin-2-yl)methyl)-N³,N³-dimethylpropane-1,3-diamine;
5-(2-((isopropyl(methyl)amino)methyl)-1H-pyrrolo[2,3-b]pyridine-4-yl)-1H-indazol-3-amine;
5-(2-(piperidin-1-ylmethyl)-1H-pyrrolo[2,3-b]pyridine-4-yl)-1H-indazol-3-amine;
5-(2-((4,4-difluoropiperidin-1-yl)methyl)-1H-pyrrolo[2,3-b]pyridine-4-yl)-1H-indazol-3-amine;
5-(2-(morpholinomethyl)-1H-pyrrolo[2,3-b]pyridine-4-yl)-1H-indazol-3-amine;
5-(2-((4-methylpiperazin-1-yl)methyl)-1H-pyrrolo[2,3-b]pyridine-4-yl)-1H-indazol-3-amine;
5-(2-((4-(tert-butyl)piperazin-1-yl)methyl)-1H-pyrrolo[2,3-b]pyridin-4-yl)-1H-indazol-3-amine;
5-(2-(azepan-1-ylmethyl)-1H-pyrrolo[2,3-b]pyridine-4-yl)-1H-indazol-3-amine;
5-(2-((4-methyl-1,4-diazepan-1-yl)methyl)-1H-pyrrolo[2,3-b]pyridin-4-yl)-1H-indazol-3-amine;
5-(2-(2-(piperidin-1-yl)ethyl)-1H-pyrrolo[2,3-b]pyridine-4-yl)-1H-indazol-3-amine;
5-(2-(2-morpholinoethyl)-1H-pyrrolo[2,3-b]pyridin-4-yl)-1H-indazol-3-amine;
5-(2-(3-(piperidin-1-yl)propyl)-1H-pyrrolo[2,3-b]pyridin-4-yl)-1H-indazol-3-amine;
5-(2-(3-(cyclohexylamino)propyl)-1H-pyrrolo[2,3-b]pyridin-4-yl)-1H-indazol-3-amine;
5-(2-(3-morpholinopropyl)-1H-pyrrolo[2,3-b]pyridin-4-yl)-1H-indazol-3-amine;
5-(2-(piperidin-4-ylmethyl)-1H-pyrrolo[2,3-b]pyridin-4-yl)-1H-indazol-3-amine;
5-(2-((1-benzylpiperidin-4-yl)methyl)-1H-pyrrolo[2,3-b]pyridin-4-yl)-1H-indazol-3-amine;
N-(4-(3-Amino-7-(3,3-dimethylbut-1-yn-1-yl)-1H-indazol-5-yl)pyridin-2-yl)oxazol-2-amine;
7-(3,3-Dimethylbut-1-yn-1-yl)-5-(2-((3,3,3-trifluoropropyl)amino)pyridin-4-yl)-1H-indazol-3-amine;
7-(Cyclopropylethynyl)-5-(2-(oxetan-3-ylamino)pyridin-4-yl)-1H-indazol-3-amine;
Methyl (4-(3-amino-7-((tetrahydro-2H-pyran-4-yl)ethynyl)-1H-indazol-5-yl)pyridin-2-yl)carbamate;
Methyl (4-(3-amino-7-((3-methyloxetan-3-yl)ethynyl)-1H-indazol-5-yl)pyridin-2-yl)carbamate;
4-(3-Amino-5-(2-(oxetan-3-ylamino)pyridin-4-yl)-1H-indazol-7-yl)-2-methylbut-3-yn-2-ol;
5-(2-(Oxetan-3-ylamino)pyridin-4-yl)-7-((tetrahydro-2H-pyran-4-yl)ethynyl)-1H-indazol-3-amine;
N-(4-(3-Amino-7-(cyclopropylethynyl)-1H-indazol-5-yl)pyridin-2-yl)cyclopropanecarboxamide;
N-(4-(3-Amino-7-(3-hydroxy-3-methylbut-1-yn-1-yl)-1H-indazol-5-yl)pyridin-2-yl)cyclopropanecarboxamide;
Methyl (4-(3-amino-7-(5-morpholinopent-1-yn-1-yl)-1H-indazol-5-yl)pyridin-2-yl)carbamate;
N-(4-(3-Amino-7-(3-hydroxy-3-methylbutyl)-1H-indazol-5-yl)pyridin-2-yl)cyclopropanecarboxamide;
Methyl (4-(3-amino-7-(3,3-dimethylbutyl)-1H-indazol-5-yl)pyridin-2-yl)carbamate;
5-(2-Cyclopropyl-1H-pyrrolo[2,3-b]pyridin-4-yl)-7-(3,3-dimethylbut-1-yn-1-yl)-1H-indazol-3-amine;
5-(2-Cyclopentyl-1H-pyrrolo[2,3-b]pyridin-4-yl)-7-(3,3-dimethylbut-1-yn-1-yl)-1H-indazol-3-amine;
5-(2-(tert-Butyl)-1H-pyrrolo[2,3-b]pyridin-4-yl)-7-(3,3-dimethylbut-1-yn-1-yl)-1H-indazol-3-amine;
(4-(3-Amino-7-(3,3-dimethylbut-1-yn-1-yl)-1H-indazol-5-yl)-1H-pyrrolo[2,3-b]pyridin-2-yl)methanol;
2-(4-(3-Amino-7-(3,3-dimethylbut-1-yn-1-yl)-1H-indazol-5-yl)-1H-pyrrolo[2,3-b]pyridin-2-yl)propan-2-ol;
Methyl 4-(3-amino-7-(3,3-dimethylbut-1-yn-1-yl)-1H-indazol-5-yl)-1H-pyrrolo[2,3-b]pyridine-2-carboxylate;
5-(2-(Difluoromethyl)-3H-imidazo[4,5-b]pyridin-7-yl)-7-(3,3-dimethylbut-1-yn-1-yl)-1H-indazol-3-amine;
5-(2-Cyclobutyl-3H-imidazo[4,5-b]pyridin-7-yl)-7-(3,3-dimethylbut-1-yn-1-yl)-1H-indazol-3-amine;
N-(4-(3-Amino-7-(3,3-dimethylbut-1-yn-1-yl)-1H-indazol-5-yl)pyridin-2-yl)cyanamide;
5-(2-((1H-Pyrazol-3-yl)amino)pyridin-4-yl)-7-(3,3-dimethylbut-1-yn-1-yl)-1H-indazol-3-amine;
5-(2-((1H-Pyrazol-4-yl)amino)pyridin-4-yl)-7-(3,3-dimethylbut-1-yn-1-yl)-1H-indazol-3-amine;
7-(3,3-Dimethylbut-1-yn-1-yl)-5-(2-((5-methyl-1H-pyrazol-3-yl)amino)pyridin-4-yl)-1H-indazol-3-amine;
7-(3,3-Dimethylbut-1-yn-1-yl)-5-(2-((3-methyl-1H-pyrazol-4-yl)amino)pyridin-4-yl)-1H-indazol-3-amine;
N-(4-(3-Amino-7-(3,3-dimethylbut-1-yn-1-yl)-1H-indazol-5-yl)pyridin-2-yl)thiazol-2-amine;
N-(4-(3-Amino-7-(3,3-dimethylbut-1-yn-1-yl)-1H-indazol-5-yl)pyridin-2-yl)-4-methyloxazol-2-amine;
N-(4-(3-Amino-7-(3,3-dimethylbut-1-yn-1-yl)-1H-indazol-5-yl)pyridin-2-yl)-4-(trifluoromethyl)oxazol-2-amine;
N-(4-(3-amino-7-(3,3-dimethylbut-1-yn-1-yl)-1H-indazol-5-yl)pyridin-2-yl)-3,5-dimethylisoxazol-4-amine;
5-(2-((1H-Imidazol-4-yl)amino)pyridin-4-yl)-7-(3,3-dimethylbut-1-yn-1-yl)-1H-indazol-3-amine;
5-(2-((4H-1,2,4-Triazol-3-yl)amino)pyridin-4-yl)-7-(3,3-dimethylbut-1-yn-1-yl)-1H-indazol-3-amine;
7-(3,3-Dimethylbut-1-yn-1-yl)-5-(2-((2-methyl-2H-tetrazol-5-yl)amino)pyridin-4-yl)-1H-indazol-3-amine;
7-(3,3-Dimethylbut-1-yn-1-yl)-5-(2-(pyrimidin-2-ylamino)pyridin-4-yl)-1H-indazol-3-amine;
7-(3,3-Dimethylbut-1-yn-1-yl)-5-(2-((tetrahydrofuran-3-yl)amino)pyridin-4-yl)-1H-indazol-3-amine;
Methyl (4-(3-Amino-7-(4-hydroxyphenyl)-1H-indazol-5-yl)pyridin-2-yl)carbamate;
4-(3-Amino-5-(2-(oxetan-3-ylamino)pyridin-4-yl)-1H-indazol-7-yl) phenol;
Methyl (4-(3-amino-7-(4-aminophenyl)-1H-indazol-5-yl)pyridin-2-yl)carbamate;
Methyl (4-(7-(4-acetamidophenyl)-3-amino-1H-indazol-5-yl)pyridin-2-yl)carbamate;
Methyl (4-(3-amino-7-(4-carbamoylphenyl)-1H-indazol-5-yl)pyridin-2-yl)carbamate;
Methyl (4-(3-amino-7-(4-(morpholinomethyl)phenyl)-1H-indazol-5-yl)pyridin-2-yl)carbamate;
5-(2-Aminopyridin-4-yl)-7-(4-(2-morpholinoethyl)phenyl)-1H-indazol-3-amine;
Methyl (4-(3-amino-7-(4-(2-morpholinoethyl)phenyl)-1H-indazol-5-yl)pyridin-2-yl)carbamate;
Methyl (4-(3-amino-7-(4-(methylsulfonyl)phenyl)-1H-indazol-5-yl)pyridin-2-yl)carbamate;
Methyl (4-(3-amino-7-(3-hydroxyphenyl)-1H-indazol-5-yl)pyridin-2-yl)carbamate;
Methyl (4-(3-amino-7-(3-carbamoylphenyl)-1H-indazol-5-yl)pyridin-2-yl)carbamate;
Methyl (4-(3-amino-7-(3-(morpholinomethyl)phenyl)-1H-indazol-5-yl)pyridin-2-yl)carbamate;
N-(4-(3-Amino-1H-indazol-5-yl)pyridin-2-yl)-2-cyclohexylacetamide;
6-Amino-4-(3-amino-7-(3,3-dimethylbut-1-yn-1-yl)-1H-indazol-5-yl)nicotinonitrile.

24. A pharmaceutical composition comprising a compound according in any anyone of claims 1 to 23, or a pharmaceutically acceptable salt or solvate thereof, and a pharmaceutically acceptable excipient.

25. A compound according to any one of claims 1 to 23, or a pharmaceutically acceptable salt or solvate thereof, or a pharmaceutical formulation according to claim 24:
(i) for use in therapy;
(ii) for use in the treatment of a disease or condition responsive to IKKalpha modulation;
(iii) for use in the treatment of a proliferative disorder (e.g. cancer); or
(iv) for use in the treatment of inflammation.
